SEQUENCE LISTING (327 sequences)

SEQ ID NO: 1 - protein, 368 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSSGGGGSGGGGSHHHHHH

SEQ ID NO: 2 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 3 - DNA, 1104 bp, Homo sapiens
  source 1..1104: mol_type = other DNA
atggaaggcatcagcatctacaccagcgacaactacaccgaggaaatgggcagcggcgactacgacagcatgaaggaaccctgcttccgggaagagaacgccaacttcaacaagatcttcctgcccacaatctacagcatcatctttctgaccggcatcgtgggcaacggactcgtgatcctcgtgatgggctaccagaaaaagctgcggagcatgaccgacaagtaccggctgcacctgagcgtggccgacctgctgttcgtgatcaccctgcctttctgggccgtggacgccgtggccaattggtacttcggcaacttcctgtgcaaggccgtgcacgtgatctacaccgtgaacctgtacagcagcgtgctgatcctggccttcatcagcctggacagatacctggccatcgtgcacgccaccaacagccagcggcctagaaagctgctggccgagaaggtggtgtacgtgggcgtgtggattcccgccctgctgctgaccatccccgacttcatcttcgccaacgtgtccgaggccgacgaccggtacatctgcgaccggttctaccccaacgacctgtgggtggtggtgttccagttccagcacatcatggtgggactgatcctgcctggcatcgtgattctgagctgctactgcatcatcatcagcaagctgagccacagcaagggccaccagaagcggaaggccctgaaaaccaccgtgatcctgattctggctttcttcgcctgctggctgccctactacatcggcatctccatcgacagcttcatcctgctggaaatcatcaagcagggctgcgagttcgagaacaccgtgcacaagtggatcagcattaccgaggccctggcctttttccactgctgcctgaaccctatcctgtacgccttcctgggcgccaagttcaagacctctgcccagcacgccctgaccagcgtgtccagaggaagcagcctgaagatcctgagcaagggcaagagaggcggccacagctccgtgtctacagagagcgagagcagcagcttccacagctctggcggcggaggatctgggggaggcggatctcaccaccatcaccatcac

SEQ ID NO: 4 - protein, 362 aa, Homo sapiens
MDLHLFDYSEPGNFSDISWPCNSSDCIVVDTVMCPNMPNKSVLLYTLSFIYIFIFVIGMIANSVVVWVNIQAKTTGYDTHCYILNLAIADLWVVLTIPVWVVSLVQHNQWPMGELTCKVTHLIFSINLFGSIFFLTCMSVDRYLSITYFTNTPSSRKKMVRRVVCILVWLLAFCVSLPDTYYLKTVTSASNNETYCRSFYPEHSIKEWLIGMELVSVVLGFAVPFSIIAVFYFLLARAISASSDQEKHSSRKIIFSYVVVFLVCWLPYHVAVLLDIFSILHYIPFTCRLEHALFTALHVTQCLSLVHCCVNPVLYSFINRNYRYELMKAFIFKYSAKTGLTKLIDASRVSETEYSALEQSTK

SEQ ID NO: 5 - protein, 362 aa, Homo sapiens; identical to SEQ ID NO: 4

SEQ ID NO: 6 - DNA, 1086 bp, Homo sapiens
  source 1..1086: mol_type = other DNA
atggacctgcacctgttcgactattccgagcctggcaacttctccgacatctcctggccttgcaactccagcgactgcatcgtggtggacaccgtgatgtgccccaacatgcccaacaagtccgtgctgctgtacaccctgtccttcatctacatcttcatcttcgtgatcggcatgatcgccaactccgtggtcgtgtgggtcaacatccaggctaagaccaccggctacgacacccactgctacatcctgaacctggctatcgccgacctgtgggtcgtgctgacaatccctgtgtgggttgtgtccctggtgcagcacaaccagtggcctatgggagagctgacctgcaaagtgacccacctgatcttctccatcaacctgttcggctctatcttcttcctgacctgtatgtccgtggaccgctacctgtccatcacctacttcaccaacacaccctccagccggaagaaaatggtccgacgggtcgtgtgcatcctcgtgtggctgctggccttctgtgtgtctctgcccgacacctactacctgaaaaccgtgacctccgcctccaacaacgagacatactgccggtctttctaccccgagcactccatcaaagaatggctgatcggaatggaactggtgtccgtggtgctgggcttcgccgtgcctttctctattatcgccgtgttctacttcctgctggccagagccatctccgcctcttccgatcaagagaagcactcctctcggaagatcatcttctcctacgtggtggtgtttctcgtgtgctggctgccttaccatgtggctgtgctgctggacatcttcagcatcctgcactacatccctttcacctgtcggctggaacacgccctgtttaccgctctgcatgtgacccagtgcctgagcctggtgcactgctgtgtgaaccctgtgctgtactccttcatcaaccggaactaccgctacgagctgatgaaggcctttatcttcaagtactccgccaagaccggcctgaccaagctgatcgacgcctccagagtgtccgagacagagtacagcgccctggaacagtccaccaag

SEQ ID NO: 7 - protein, 352 aa, Macaca fascicularis
MEGISIYTSDNYTEEMGSGDYDSIKEPCFREENAHFNRIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQKPRKLLAEKVVYVGVWIPALLLTIPDFIFASVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 8 - protein, 352 aa, Macaca fascicularis; identical to SEQ ID NO: 7

SEQ ID NO: 9 - DNA, 1056 bp, Macaca fascicularis
  source 1..1056: mol_type = other DNA
atggaaggcatctccatctacacctccgacaactacaccgaggaaatgggctccggcgactacgactccatcaaagagccttgcttccgggaagagaacgcccacttcaaccggatctttctgcccacaatctactctatcatcttcctgaccggcatcgtcggcaacggcctggtcattctggtcatgggctaccagaaaaagctgcggtccatgaccgacaagtaccggctgcatctgtctgtggccgacctgctgttcgtgatcaccctgcctttttgggccgtcgatgccgtggccaactggtacttcggcaacttcctgtgcaaggccgtgcacgtgatctacaccgtgaacctgtactcctccgtgctgatcctggccttcatctccctggacagatacctggccatcgtgcacgccaccaactctcagaagcccagaaagctgctggccgagaaggtggtgtatgtcggcgtgtggattcccgctctgctgctgacaatccccgacttcatcttcgcctccgtgtccgaggccgacgaccggtacatctgcgacagattctaccccaacgacctgtgggtcgtcgtgttccagttccagcacatcatggtcggactgatcctgcctggcatcgtgatcctgtcctgctactgcatcatcatctccaagctgtcccactccaagggccaccagaagcggaaggctctgaaaaccacagtgatcctgattctggctttcttcgcctgctggctgccctactacatcggcatcagcatcgacagcttcatcctgctggaaatcatcaagcagggctgcgagttcgagaacaccgtgcacaagtggatctccattaccgaggctctggccttcttccactgttgtctgaaccctatcctgtacgccttcctgggcgccaagttcaagacctctgctcagcacgctctgacctctgtgtccagaggctccagcctgaagatcctgagcaagggcaagagaggcggccactcttctgtgtctaccgagtccgagtcctccagcttccactcctct

SEQ ID NO: 10 - protein, 8 aa, synthetic construct
GFTFYSYG

SEQ ID NO: 11 - protein, 8 aa, synthetic construct
IWYGGTNK

SEQ ID NO: 12 - protein, 14 aa, synthetic construct
ARGQLERRRGGLNI

SEQ ID NO: 13 - protein, 5 aa, synthetic construct
SYGMH

SEQ ID NO: 14 - protein, 17 aa, synthetic construct
VIWYGGTNKYYADSVKG

SEQ ID NO: 15 - protein, 12 aa, synthetic construct
GQLERRRGGLNI

SEQ ID NO: 16 - protein, 121 aa, synthetic construct
QVQLVESGGGVVQPGRSLRLSCAASGFTFYSYGMHWVRQAPGKGLEWVAVIWYGGTNKYYADSVKGRFTISRDNSKNTLYLQMNSLTAEDTAVYYCARGQLERRRGGLNIWGQGTVVTVSS

SEQ ID NO: 17 - DNA, 364 bp, synthetic construct
  source 1..364: mol_type = other DNA
caggtgcagctggtggagtctgggggaggcgtggtccagcctgggaggtccctgagactctcctgtgcagcgtctggattcaccttctatagctatggcatgcactgggtccgccaggctccaggcaagggactggagtgggtggcagttatatggtatggtggaactaataaatactatgcagactccgtgaagggccgattcaccatctccagagacaattccaagaacacgctgtatctgcaaatgaacagcctgacagccgaagacacggctgtgtattactgtgcgagagggcagctggaacgccgacggggcggtttgaatatctggggccaagggacagtggtcaccgtctcttcag

SEQ ID NO: 18 - protein, 448 aa, synthetic construct
QVQLVESGGGVVQPGRSLRLSCAASGFTFYSYGMHWVRQAPGKGLEWVAVIWYGGTNKYYADSVKGRFTISRDNSKNTLYLQMNSLTAEDTAVYYCARGQLERRRGGLNIWGQGTVVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - DNA, 1344 bp, synthetic construct
  source 1..1344: mol_type = other DNA
caggtgcagctggtggagtctgggggaggcgtggtccagcctgggaggtccctgagactctcctgtgcagcgtctggattcaccttctatagctatggcatgcactgggtccgccaggctccaggcaagggactggagtgggtggcagttatatggtatggtggaactaataaatactatgcagactccgtgaagggccgattcaccatctccagagacaattccaagaacacgctgtatctgcaaatgaacagcctgacagccgaagacacggctgtgtattactgtgcgagagggcagctggaacgccgacggggcggtttgaatatctggggccaagggacagtggtcaccgtctcttcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 20 - protein, 6 aa, synthetic construct
QGVRDD

SEQ ID NO: 21 - skipped

SEQ ID NO: 22 - protein, 9 aa, synthetic construct
LQHNSYPFT

SEQ ID NO: 23 - protein, 11 aa, synthetic construct
RASQGVRDDLG

SEQ ID NO: 24 - protein, 7 aa, synthetic construct
AASSLQS

SEQ ID NO: 25 - protein, 9 aa, synthetic construct
LQHNSYPFT

SEQ ID NO: 26 - protein, 107 aa, synthetic construct
DIQMTQSPSSLSASVGDRVTITCRASQGVRDDLGWYQQKPGKAPKRLIYAASSLQSGVPSRFSGSGSGTEFTLTISSLQPEDFATYYCLQHNSYPFTFGPGTKVDIK

SEQ ID NO: 27 - DNA, 322 bp, synthetic construct
  source 1..322: mol_type = other DNA
gacatccagatgacccagtctccatcctccctgtctgcatctgtaggagacagagtcaccatcacttgccgggcaagtcagggcgttagagatgatttaggttggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttatccattcactttcggccctgggaccaaagtggatatcaaac

SEQ ID NO: 28 - protein, 214 aa, synthetic construct
DIQMTQSPSSLSASVGDRVTITCRASQGVRDDLGWYQQKPGKAPKRLIYAASSLQSGVPSRFSGSGSGTEFTLTISSLQPEDFATYYCLQHNSYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - DNA, 642 bp, synthetic construct
  source 1..642: mol_type = other DNA
gacatccagatgacccagtctccatcctccctgtctgcatctgtaggagacagagtcaccatcacttgccgggcaagtcagggcgttagagatgatttaggttggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttatccattcactttcggccctgggaccaaagtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 30 - protein, 8 aa, synthetic construct
GFTFYSYA

SEQ ID NO: 31 - protein, 8 aa, synthetic construct
ISGSGDIT

SEQ ID NO: 32 - protein, 17 aa, synthetic construct
AKGQLERRYYKYYGMDV

SEQ ID NO: 33 - protein, 5 aa, synthetic construct
SYAMN

SEQ ID NO: 34 - protein, 17 aa, synthetic construct
GISGSGDITYYADSVKG

SEQ ID NO: 35 - protein, 15 aa, synthetic construct
GQLERRYYKYYGMDV

SEQ ID NO: 36 - protein, 124 aa, synthetic construct
EVQLVESGGDLVQPGGSLRLSCAASGFTFYSYAMNWVRQAPGKGLKWVSGISGSGDITYYADSVKGRFTISRDNSKNTLYLQMHSLRPEDTAIYYCAKGQLERRYYKYYGMDVWGQGTTVTVSS

SEQ ID NO: 37 - DNA, 373 bp, synthetic construct
  source 1..373: mol_type = other DNA
gaggtgcagctggtagagtctgggggagacttagtacagcctggggggtccctgagactctcttgtgcagcctctggattcaccttttacagctatgccatgaattgggtccgccaggctccagggaaggggctaaaatgggtctcaggtattagtggtagtggtgatatcacatactacgcagactccgtgaagggccggttcaccatctccagagacaattccaagaacacgctgtatctgcaaatgcacagcctgagacccgaagacacggccatatattactgtgcgaaaggccaactggaacgacggtactacaagtactacggtatggacgtctggggccaagggaccacggtcaccgtctcctcag

SEQ ID NO: 38 - protein, 451 aa, synthetic construct
EVQLVESGGDLVQPGGSLRLSCAASGFTFYSYAMNWVRQAPGKGLKWVSGISGSGDITYYADSVKGRFTISRDNSKNTLYLQMHSLRPEDTAIYYCAKGQLERRYYKYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 39 - DNA, 1353 bp, synthetic construct
  source 1..1353: mol_type = other DNA
gaggtgcagctggtagagtctgggggagacttagtacagcctggggggtccctgagactctcttgtgcagcctctggattcaccttttacagctatgccatgaattgggtccgccaggctccagggaaggggctaaaatgggtctcaggtattagtggtagtggtgatatcacatactacgcagactccgtgaagggccggttcaccatctccagagacaattccaagaacacgctgtatctgcaaatgcacagcctgagacccgaagacacggccatatattactgtgcgaaaggccaactggaacgacggtactacaagtactacggtatggacgtctggggccaagggaccacggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 40 - protein, 6 aa, synthetic construct
QGIRND

SEQ ID NO: 41 - skipped

SEQ ID NO: 42 - protein, 9 aa, synthetic construct
LQQNSYPLT

SEQ ID NO: 43 - protein, 11 aa, synthetic construct
RANQGIRNDLG

SEQ ID NO: 44 - protein, 7 aa, synthetic construct
GASSLKS

SEQ ID NO: 45 - protein, 9 aa, synthetic construct
LQQNSYPLT

SEQ ID NO: 46 - protein, 107 aa, synthetic construct
DIQMTQSPSSLSASVGDRVTITCRANQGIRNDLGWYQQKPGKAPKRLIYGASSLKSGVPSRFSGSGSGTEFTLTISSLQPEDFATYYCLQQNSYPLTFGGGTKVEIK

SEQ ID NO: 47 - DNA, 322 bp, synthetic construct
  source 1..322: mol_type = other DNA
gacatccagatgacccagtctccatcctccctgtctgcatctgtaggagacagagtcaccatcacttgccgggcaaatcagggcattagaaatgatttaggctggtatcagcagaaaccagggaaagcccctaagcgcctgatctatggtgcatccagtttgaaaagtggggtcccatcaaggttcagcggcagtggatctgggacagaattcactctcacaatcagcagcctgcagcctgaagattttgcaacctactattgtctacagcagaatagttacccgctcactttcggcggagggaccaaagtggagatcaaac

SEQ ID NO: 48 - protein, 214 aa, synthetic construct
DIQMTQSPSSLSASVGDRVTITCRANQGIRNDLGWYQQKPGKAPKRLIYGASSLKSGVPSRFSGSGSGTEFTLTISSLQPEDFATYYCLQQNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - DNA, 642 bp, synthetic construct
  source 1..642: mol_type = other DNA
gacatccagatgacccagtctccatcctccctgtctgcatctgtaggagacagagtcaccatcacttgccgggcaaatcagggcattagaaatgatttaggctggtatcagcagaaaccagggaaagcccctaagcgcctgatctatggtgcatccagtttgaaaagtggggtcccatcaaggttcagcggcagtggatctgggacagaattcactctcacaatcagcagcctgcagcctgaagattttgcaacctactattgtctacagcagaatagttacccgctcactttcggcggagggaccaaagtggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 50 - protein, 10 aa, synthetic construct
GGSISSGGYY

SEQ ID NO: 51 - protein, 7 aa, synthetic construct
ISYSGNT

SEQ ID NO: 52 - protein, 21 aa, synthetic construct
ARDRRIPMLRGVKGYYYGLDV

SEQ ID NO: 53 - protein, 7 aa, synthetic construct
SGGYYWS

SEQ ID NO: 54 - protein, 16 aa, synthetic construct
YISYSGNTFYSPSLKS

SEQ ID NO: 55 - protein, 19 aa, synthetic construct
DRRIPMLRGVKGYYYGLDV

SEQ ID NO: 56 - protein, 129 aa, synthetic construct
QVQLQESGPGLVKPSQTLSLTCTVSGGSISSGGYYWSWIRQVPGKGLEWIGYISYSGNTFYSPSLKSRVTISVDTSENKFSLRLTSVTAADTAVYFCARDRRIPMLRGVKGYYYGLDVWGQGTTVTVSS

SEQ ID NO: 57 - DNA, 388 bp, synthetic construct
  source 1..388: mol_type = other DNA
caggtgcagctgcaggagtcgggcccaggactggtgaagccttcacagaccctgtccctcacctgcactgtctctggtggctccatcagcagtggtgggtactactggagctggatccgccaggtcccagggaagggcctggagtggattggatacatctcatacagtgggaacaccttctacagcccgtccctcaagagtcgagttaccatatcagtagacacgtctgagaacaagttctccctgaggctgacctctgtgactgccgcggacacggccgtgtatttctgtgcgagagatcgacgtattcctatgcttcggggagttaaaggctactactacggtttggacgtctggggccaagggaccacggtcaccgtctcctcag

SEQ ID NO: 58 - protein, 456 aa, synthetic construct
QVQLQESGPGLVKPSQTLSLTCTVSGGSISSGGYYWSWIRQVPGKGLEWIGYISYSGNTFYSPSLKSRVTISVDTSENKFSLRLTSVTAADTAVYFCARDRRIPMLRGVKGYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - DNA, 1368 bp, synthetic construct
  source 1..1368: mol_type = other DNA
caggtgcagctgcaggagtcgggcccaggactggtgaagccttcacagaccctgtccctcacctgcactgtctctggtggctccatcagcagtggtgggtactactggagctggatccgccaggtcccagggaagggcctggagtggattggatacatctcatacagtgggaacaccttctacagcccgtccctcaagagtcgagttaccatatcagtagacacgtctgagaacaagttctccctgaggctgacctctgtgactgccgcggacacggccgtgtatttctgtgcgagagatcgacgtattcctatgcttcggggagttaaaggctactactacggtttggacgtctggggccaagggaccacggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 60 - protein, 11 aa, synthetic construct
QSLLHRNGYKY

SEQ ID NO: 61 - skipped

SEQ ID NO: 62 - protein, 9 aa, synthetic construct
MQALQTPWT

SEQ ID NO: 63 - protein, 16 aa, synthetic construct
RSSQSLLHRNGYKYLD

SEQ ID NO: 64 - protein, 7 aa, synthetic construct
LGSNRAS

SEQ ID NO: 65 - protein, 9 aa, synthetic construct
MQALQTPWT

SEQ ID NO: 66 - protein, 112 aa, synthetic construct
DIVMTQSPLSLPVIPGEPASISCRSSQSLLHRNGYKYLDWYLQKPGQSPQLLIYLGSNRASGVPARFSGSGSGTDFTLKISRVEAEDVGVYYCMQALQTPWTFGQGTKVEIK

SEQ ID NO: 67 - DNA, 337 bp, synthetic construct
  source 1..337: mol_type = other DNA
gatattgtgatgactcagtctccactctccctgcccgtcatccctggagagccggcctccatctcctgcaggtctagtcagagcctcctgcatagaaatggatacaagtatttggattggtacctgcagaagccggggcagtctccacagctcctgatctatttgggttctaatcgggcctccggggtccctgccaggttcagtggcagtggatcaggcacagattttacactgaaaatcagcagagtggaggctgaggatgttggggtttattactgtatgcaagctctacaaactccgtggacgttcggccaagggaccaaggtggaaatcaaac

SEQ ID NO: 68 - protein, 219 aa, synthetic construct
DIVMTQSPLSLPVIPGEPASISCRSSQSLLHRNGYKYLDWYLQKPGQSPQLLIYLGSNRASGVPARFSGSGSGTDFTLKISRVEAEDVGVYYCMQALQTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - DNA, 657 bp, synthetic construct
  source 1..657: mol_type = other DNA
gatattgtgatgactcagtctccactctccctgcccgtcatccctggagagccggcctccatctcctgcaggtctagtcagagcctcctgcatagaaatggatacaagtatttggattggtacctgcagaagccggggcagtctccacagctcctgatctatttgggttctaatcgggcctccggggtccctgccaggttcagtggcagtggatcaggcacagattttacactgaaaatcagcagagtggaggctgaggatgttggggtttattactgtatgcaagctctacaaactccgtggacgttcggccaagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 70 - protein, 8 aa, synthetic construct
GFSFSSYG

SEQ ID NO: 71 - protein, 8 aa, synthetic construct
ISYDGSNK

SEQ ID NO: 72 - protein, 19 aa, synthetic construct
SKGSLTPRRGYYYSYGMDV

SEQ ID NO: 73 - protein, 5 aa, synthetic construct
SYGMH

SEQ ID NO: 74 - protein, 17 aa, synthetic construct
VISYDGSNKYYADSVKG

SEQ ID NO: 75 - protein, 17 aa, synthetic construct
GSLTPRRGYYYSYGMDV

SEQ ID NO: 76 - protein, 126 aa, synthetic construct
QVQLVESGGGVVQPGRSLRLSCAASGFSFSSYGMHWVRQAPGKGLEWVAVISYDGSNKYYADSVKGRFTISRDNSKNTLYLQMNSLRGEDTAVYYCSKGSLTPRRGYYYSYGMDVWGQGTTVTVSS

SEQ ID NO: 77 - DNA, 379 bp, synthetic construct
  source 1..379: mol_type = other DNA
caggtgcagctggtggagtctgggggaggcgtggtccagcctgggaggtccctgagactctcctgtgcagcctctggattctccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagaggtgaggacacggctgtgtattactgttcgaaagggagtttaacacctcgccggggttactactactcctacggtatggacgtctggggccaagggaccacggtcaccgtctcctcag

SEQ ID NO: 78 - protein, 453 aa, synthetic construct
QVQLVESGGGVVQPGRSLRLSCAASGFSFSSYGMHWVRQAPGKGLEWVAVISYDGSNKYYADSVKGRFTISRDNSKNTLYLQMNSLRGEDTAVYYCSKGSLTPRRGYYYSYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 79 - DNA, 1359 bp, synthetic construct
  source 1..1359: mol_type = other DNA
caggtgcagctggtggagtctgggggaggcgtggtccagcctgggaggtccctgagactctcctgtgcagcctctggattctccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagaggtgaggacacggctgtgtattactgttcgaaagggagtttaacacctcgccggggttactactactcctacggtatggacgtctggggccaagggaccacggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 80 - protein, 6 aa, synthetic construct
QGIRND

SEQ ID NO: 81 - skipped

SEQ ID NO: 82 - protein, 9 aa, synthetic construct
LQHNGYPFT

SEQ ID NO: 83 - protein, 11 aa, synthetic construct
RASQGIRNDLS

SEQ ID NO: 84 - protein, 7 aa, synthetic construct
AASSLQS

SEQ ID NO: 85 - protein, 9 aa, synthetic construct
LQHNGYPFT

SEQ ID NO: 86 - protein, 107 aa, synthetic construct
DIQMTQSPSSLSASVGDRVTITCRASQGIRNDLSWYQQKPGKAPKRLIYAASSLQSGVPSRFSGSGSGTEFTLTISSLQPEDFATYYCLQHNGYPFTFGPGTKVDIK

SEQ ID NO: 87 - DNA, 322 bp, synthetic construct
  source 1..322: mol_type = other DNA
gacatccagatgacccagtctccatcctccctgtctgcatctgtaggagacagagtcaccatcacttgccgggcaagtcagggcattagaaatgatttaagctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatggttacccattcactttcggccctgggaccaaagtggatatcaaac

SEQ ID NO: 88 - protein, 214 aa, synthetic construct
DIQMTQSPSSLSASVGDRVTITCRASQGIRNDLSWYQQKPGKAPKRLIYAASSLQSGVPSRFSGSGSGTEFTLTISSLQPEDFATYYCLQHNGYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - DNA, 642 bp, synthetic construct
  source 1..642: mol_type = other DNA
gacatccagatgacccagtctccatcctccctgtctgcatctgtaggagacagagtcaccatcacttgccgggcaagtcagggcattagaaatgatttaagctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatggttacccattcactttcggccctgggaccaaagtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 90 - protein, 8 aa, synthetic construct
GFTFSGSA

SEQ ID NO: 91 - protein, 10 aa, synthetic construct
IRSKANKYAT

SEQ ID NO: 92 - protein, 19 aa, synthetic construct
TWELSNYDILTGFYYGMDV

SEQ ID NO: 93 - protein, 5 aa, synthetic construct
GSAMH

SEQ ID NO: 94 - protein, 19 aa, synthetic construct
RIRSKANKYATAYAASVKG

SEQ ID NO: 95 - protein, 17 aa, synthetic construct
ELSNYDILTGFYYGMDV

SEQ ID NO: 96 - protein, 128 aa, synthetic construct
EVQLVESGGGLVQPGGSLKLSCAASGFTFSGSAMHWVRQASGKGLEWVGRIRSKANKYATAYAASVKGRFTISRDDSKNTAYVQMKSLKTEDTAVYYCTWELSNYDILTGFYYGMDVWGQGTTVTVSS

SEQ ID NO: 97 - DNA, 385 bp, synthetic construct
  source 1..385: mol_type = other DNA
gaggtgcagctggtggagtccgggggaggcttggtccagccgggggggtccctgaaactctcctgtgcagcctctgggttcaccttcagtggatctgctatgcactgggtccgccaggcttccgggaaagggctggagtgggttggccgtattagaagtaaagctaacaaatatgcgacagcatatgctgcgtcggtgaaaggcaggttcaccatctccagagatgattcaaagaatacggcgtatgtgcaaatgaagagcctgaaaaccgaggacacggccgtgtattactgtacttgggaattgtcgaattacgatattttgactggtttctactacggtatggacgtctggggccaagggaccacggtcaccgtctcctcag

SEQ ID NO: 98 - protein, 455 aa, synthetic construct
EVQLVESGGGLVQPGGSLKLSCAASGFTFSGSAMHWVRQASGKGLEWVGRIRSKANKYATAYAASVKGRFTISRDDSKNTAYVQMKSLKTEDTAVYYCTWELSNYDILTGFYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 99 - DNA, 1365 bp, synthetic construct
  source 1..1365: mol_type = other DNA
gaggtgcagctggtggagtccgggggaggcttggtccagccgggggggtccctgaaactctcctgtgcagcctctgggttcaccttcagtggatctgctatgcactgggtccgccaggcttccgggaaagggctggagtgggttggccgtattagaagtaaagctaacaaatatgcgacagcatatgctgcgtcggtgaaaggcaggttcaccatctccagagatgattcaaagaatacggcgtatgtgcaaatgaagagcctgaaaaccgaggacacggccgtgtattactgtacttgggaattgtcgaattacgatattttgactggtttctactacggtatggacgtctggggccaagggaccacggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 100 - protein, 8 aa, synthetic construct
SNNVGNLG

SEQ ID NO: 101 - skipped

SEQ ID NO: 102 - protein, 11 aa, synthetic construct
SAWDSSLSAWV

SEQ ID NO: 103 - protein, 13 aa, synthetic construct
TGNSNNVGNLGAA

SEQ ID NO: 104 - protein, 7 aa, synthetic construct
RNNNRPS

SEQ ID NO: 105 - protein, 11 aa, synthetic construct
SAWDSSLSAWV

SEQ ID NO: 106 - protein, 110 aa, synthetic construct
QAGLTQPPSVSKGLRQTATLTCTGNSNNVGNLGAAWLQQHQGHPPKLLSYRNNNRPSGISERFFASRSGNTASLTITGLQPEDEADYYCSAWDSSLSAWVFGGGTKLTVL

SEQ ID NO: 107 - DNA, 331 bp, synthetic construct
  source 1..331: mol_type = other DNA
caggcagggctgactcagccaccctcggtgtccaagggcttgagacagaccgccacactcacctgcactgggaacagcaacaatgttggcaacctaggagcagcttggctgcagcagcaccagggccaccctcccaaactcctatcctacaggaataataaccggccctcagggatctcagagagattctttgcatccaggtcaggaaacacagcctccctgaccattactggactccagcctgaggacgaggctgactattactgctcagcatgggacagcagcctcagtgcttgggtgttcggcggagggaccaagctgaccgtcctag

SEQ ID NO: 108 - protein, 216 aa, synthetic construct
QAGLTQPPSVSKGLRQTATLTCTGNSNNVGNLGAAWLQQHQGHPPKLLSYRNNNRPSGISERFFASRSGNTASLTITGLQPEDEADYYCSAWDSSLSAWVFGGGTKLTVLGQPKAAPSVTLFPPSSEELQANKATLVCLISDFSPGAVTVAWKADSSPVKAGVETTTPSKQSNNKYAASSYLSLTPEQWKSHKSYSCQVTHEGSTVKKTVAPTECS

SEQ ID NO: 109 - DNA, 648 bp, synthetic construct
  source 1..648: mol_type = other DNA
caggcagggctgactcagccaccctcggtgtccaagggcttgagacagaccgccacactcacctgcactgggaacagcaacaatgttggcaacctaggagcagcttggctgcagcagcaccagggccaccctcccaaactcctatcctacaggaataataaccggccctcagggatctcagagagattctttgcatccaggtcaggaaacacagcctccctgaccattactggactccagcctgaggacgaggctgactattactgctcagcatgggacagcagcctcagtgcttgggtgttcggcggagggaccaagctgaccgtcctaggtcagcccaaggctgccccctcggtcactctgttcccaccctcctctgaggagcttcaagccaacaaggccacactggtgtgtctcataagtgacttctccccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtgaagaagacagtggcccctacagaatgttca

SEQ ID NO: 110 - protein, 216 aa, synthetic construct
QAGLTQPPSVSKGLRQTATLTCTGNSNNVGNLGAAWLQQHQGHPPKLLSYRNNNRPSGISERFFASRSGNTASLTITGLQPEDEADYYCSAWDSSLSAWVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 111 - DNA, 648 bp, synthetic construct
  source 1..648: mol_type = other DNA
caagctggattgacacagcctcctagcgtgtccaagggcctgagacagaccgctacactgacctgcaccggcaactctaacaacgtgggcaatctgggcgctgcctggttgcagcagcatcagggacatcctccaaagctgctgtcctaccggaacaacaaccggccttccggcatctccgagcggttcttcgcttctagatccggcaataccgccagcctgaccatcactggactgcagcctgaggacgaggccgactactactgttctgcctgggactcctctctgtccgcctgggttttcggcggaggcacaaaactgacagtgctgggccagcctaaggccgctccttccgttacactgttccctccatcctccgaggaactgcaggccaacaaggctaccctcgtgtgcctgatctccgacttttaccctggcgctgtgaccgtggcctggaaggctgatagttctcctgtgaaggccggcgtggaaaccaccacaccttccaagcagtccaacaacaaatacgccgcctcctcctacctgtctctgacccctgaacagtggaagtcccacaagtcctacagctgccaagtgacccacgagggctccaccgtggaaaagacagtggctcctaccgagtgctcc

SEQ ID NO: 112 - protein, 121 aa, synthetic construct
QVQLVESGGGVVQPGRSLRLSCAASGFTFYSYGMHWVRQAPGKGLEWVAVIWYGGTNKYYADSVKGRFTISRDNSKNTLYLQMNSLTAEDTAVYYCARGQLERRRGGLNIWGQGTMVTVSS

SEQ ID NO: 113 - DNA, 364 bp, synthetic construct
  source 1..364: mol_type = other DNA
caggtgcagctggtggagtctgggggaggcgtggtccagcctgggaggtccctgagactctcctgtgcagcgtctggattcaccttctatagctatggcatgcactgggtccgccaggctccaggcaaggggctggagtgggtggcagttatatggtatggtggaactaataaatactatgcagactccgtgaagggccgattcaccatctccagagacaattccaagaacacgctgtatctgcaaatgaacagcctgacagccgaagacacggctgtgtattactgtgcgagagggcaactggaacgccgacggggcggtttgaatatctggggccaagggacaatggtcaccgtctcttcag

SEQ ID NO: 114 - protein, 448 aa, synthetic construct
QVQLVESGGGVVQPGRSLRLSCAASGFTFYSYGMHWVRQAPGKGLEWVAVIWYGGTNKYYADSVKGRFTISRDNSKNTLYLQMNSLTAEDTAVYYCARGQLERRRGGLNIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 115 - DNA, 1344 bp, synthetic construct
  source 1..1344: mol_type = other DNA
caggtgcagctggtggagtctgggggaggcgtggtccagcctgggaggtccctgagactctcctgtgcagcgtctggattcaccttctatagctatggcatgcactgggtccgccaggctccaggcaaggggctggagtgggtggcagttatatggtatggtggaactaataaatactatgcagactccgtgaagggccgattcaccatctccagagacaattccaagaacacgctgtatctgcaaatgaacagcctgacagccgaagacacggctgtgtattactgtgcgagagggcaactggaacgccgacggggcggtttgaatatctggggccaagggacaatggtcaccgtctcttcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 116 - protein, 107 aa, synthetic construct; identical to SEQ ID NO: 26

SEQ ID NO: 117 - DNA, 322 bp, synthetic construct; identical to SEQ ID NO: 27
  source 1..322: mol_type = other DNA

SEQ ID NO: 118 - protein, 214 aa, synthetic construct; identical to SEQ ID NO: 28

SEQ ID NO: 119 - DNA, 642 bp, synthetic construct; identical to SEQ ID NO: 29
  source 1..642: mol_type = other DNA

SEQ ID NO: 120 - protein, 130 aa, synthetic construct
QVQLVQSGAEVKKPGASVKVSCRASGYTFTNYDINWVRQATGQGLEWMGWMNPNSGNTGYAQKFQGRVTMTRNTSISAAYMELSSLRSEDTAVYYCARGRGYYGSGSRYRGYYYYGMDVWGQGTTVTVSS

SEQ ID NO: 121 - DNA, 391 bp, synthetic construct
  source 1..391: mol_type = other DNA
caggtgcagctggtgcagtctggggctgaggtgaagaagcctggggcctcagtgaaggtctcctgcagggcttctggatacaccttcaccaattatgatatcaactgggtgcgacaggccactggacaagggcttgagtggatgggatggatgaaccctaacagtggtaacacaggctatgcacagaagttccagggcagagtcaccatgaccaggaacacctccataagcgcagcctacatggagctgagcagcctgagatctgaagacacggccgtgtattactgtgcgagaggccgggggtactatggttcggggagccgttataggggctactactactacggtatggacgtctggggccaagggaccacggtcaccgtctcctcag

SEQ ID NO: 122 - protein, 457 aa, synthetic construct
QVQLVQSGAEVKKPGASVKVSCRASGYTFTNYDINWVRQATGQGLEWMGWMNPNSGNTGYAQKFQGRVTMTRNTSISAAYMELSSLRSEDTAVYYCARGRGYYGSGSRYRGYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 123 - DNA, 1371 bp, synthetic construct
  source 1..1371: mol_type = other DNA
caggtgcagctggtgcagtctggggctgaggtgaagaagcctggggcctcagtgaaggtctcctgcagggcttctggatacaccttcaccaattatgatatcaactgggtgcgacaggccactggacaagggcttgagtggatgggatggatgaaccctaacagtggtaacacaggctatgcacagaagttccagggcagagtcaccatgaccaggaacacctccataagcgcagcctacatggagctgagcagcctgagatctgaagacacggccgtgtattactgtgcgagaggccgggggtactatggttcggggagccgttataggggctactactactacggtatggacgtctggggccaagggaccacggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 124 - protein, 107 aa, synthetic construct
DIQMTQSPSTLSASVGDRVTITCRASQSISSWLAWYQQKPGKAPKVLIYKASSLESGVPSRFSGSGSGTEFTLTISSLQPDDFATYYCQQYNSYSWTFGQGTKVEIK

SEQ ID NO: 125 - DNA, 322 bp, synthetic construct
  source 1..322: mol_type = other DNA
gacatccagatgacccagtctccttccaccctgtctgcatctgtaggagacagagtcaccatcacttgccgggccagtcagagtattagtagctggttggcctggtatcagcagaaaccagggaaagcccctaaggtcctgatctataaggcgtctagtttagaaagtggggtcccatcaaggttcagcggcagtggatctgggacagaattcactctcaccatcagcagcctgcagcctgatgattttgcaacttattactgccaacagtataatagttattcgtggacgttcggccaagggaccaaggtggaaatcaaac

SEQ ID NO: 126 - protein, 214 aa, synthetic construct
DIQMTQSPSTLSASVGDRVTITCRASQSISSWLAWYQQKPGKAPKVLIYKASSLESGVPSRFSGSGSGTEFTLTISSLQPDDFATYYCQQYNSYSWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - DNA, 642 bp, synthetic construct
  source 1..642: mol_type = other DNA
gacatccagatgacccagtctccttccaccctgtctgcatctgtaggagacagagtcaccatcacttgccgggccagtcagagtattagtagctggttggcctggtatcagcagaaaccagggaaagcccctaaggtcctgatctataaggcgtctagtttagaaagtggggtcccatcaaggttcagcggcagtggatctgggacagaattcactctcaccatcagcagcctgcagcctgatgattttgcaacttattactgccaacagtataatagttattcgtggacgttcggccaagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 128 - protein, 129 aa, synthetic construct
QVQLQESGPGLVKPSQTLSLTCTVSGGSISSGGYYWTWIRQLPGKGLEWIGYMSYSGNTFYSPSLKSRFTISGDTSENQFSLKVNSVTSADTAIYYCARDRRIPMLRGIKGYYYGMDVWGQGTTVTVSS

SEQ ID NO: 129 - DNA, 388 bp, synthetic construct
  source 1..388: mol_type = other DNA
caggtgcagctgcaggagtcgggcccaggactggtgaagccttcacagaccctgtccctcacctgcactgtctctggtggctccatcagcagtggtggatactactggacctggatccgccagctcccagggaagggcctggagtggattgggtacatgtcatacagtgggaacaccttctacagcccgtccctcaagagtcgatttacaatatcaggagacacgtctgagaaccagttctccctgaaggtgaactctgtgacttccgcggacacggccatttattactgtgcgagagatcgacgtattcctatgcttcgcggaattaaaggctactactacggtatggacgtctggggccaagggaccacggtcaccgtctcctcag

SEQ ID NO: 130 - protein, 456 aa, synthetic construct
QVQLQESGPGLVKPSQTLSLTCTVSGGSISSGGYYWTWIRQLPGKGLEWIGYMSYSGNTFYSPSLKSRFTISGDTSENQFSLKVNSVTSADTAIYYCARDRRIPMLRGIKGYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 131 - DNA, 1368 bp, synthetic construct
  source 1..1368: mol_type = other DNA
caggtgcagctgcaggagtcgggcccaggactggtgaagccttcacagaccctgtccctcacctgcactgtctctggtggctccatcagcagtggtggatactactggacctggatccgccagctcccagggaagggcctggagtggattgggtacatgtcatacagtgggaacaccttctacagcccgtccctcaagagtcgatttacaatatcaggagacacgtctgagaaccagttctccctgaaggtgaactctgtgacttccgcggacacggccatttattactgtgcgagagatcgacgtattcctatgcttcgcggaattaaaggctactactacggtatggacgtctggggccaagggaccacggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 132 - protein, 112 aa, synthetic construct
DIVMTQSPLSLPVIPGEPASISCRSSQSLLHSNGYNYLDWYLQKPGQSPQLLIYLGSNRASGVPDRFRGSGSGTDFTLKIRRVEAEDVGVYYCMQALQTPWTFGQGTKVEIK

SEQ ID NO: 133 - DNA, 337 bp, synthetic construct
  source 1..337: mol_type = other DNA
gatattgtgatgactcagtctccactctccctgcccgtcatccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttccgtggcagtggatcaggcacagattttacactgaaaatcagaagagtggaggctgaggatgttggggtttattactgcatgcaagctctacagactccgtggacgttcggccaagggaccaaggtggaaatcaaac

SEQ ID NO: 134 - protein, 219 aa, synthetic construct
DIVMTQSPLSLPVIPGEPASISCRSSQSLLHSNGYNYLDWYLQKPGQSPQLLIYLGSNRASGVPDRFRGSGSGTDFTLKIRRVEAEDVGVYYCMQALQTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - DNA, 657 bp, synthetic construct
  source 1..657: mol_type = other DNA
gatattgtgatgactcagtctccactctccctgcccgtcatccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttccgtggcagtggatcaggcacagattttacactgaaaatcagaagagtggaggctgaggatgttggggtttattactgcatgcaagctctacagactccgtggacgttcggccaagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 136 - protein, 128 aa, synthetic construct; identical to SEQ ID NO: 96

SEQ ID NO: 137 - DNA, 385 bp, synthetic construct; identical to SEQ ID NO: 97
  source 1..385: mol_type = other DNA

SEQ ID NO: 138 - protein, 455 aa, synthetic construct; identical to SEQ ID NO: 98

SEQ ID NO: 139 - DNA, 1365 bp, synthetic construct; identical to SEQ ID NO: 99
  source 1..1365: mol_type = other DNA

SEQ ID NO: 140 - protein, 107 aa, synthetic construct
DIQMTQSPSTLSASVGDRVTITCRASQSISNWLAWYQQKPGKAPKVLIYKASSLESGVPSRFSGSGSGTEFTLTISSLQPDDFATYYCQQYNSFSWTFGQGTKVEIK

SEQ ID NO: 141 - DNA, 322 bp, synthetic construct
  source 1..322: mol_type = other DNA
gacatccagatgacccagtctccttccaccctgtctgcatctgtaggagacagagtcaccatcacttgccgggccagtcagagtattagtaattggttggcctggtatcagcagaaaccagggaaagcccctaaggtcctgatctataaggcgtctagtttagaaagtggggtcccatcaaggttcagcggcagtggatctgggacagaattcactctcaccatcagcagcctgcagcctgatgattttgcaacttattactgccaacagtataatagtttttcgtggacgttcggccaagggaccaaggtggaaatcaaac

SEQ ID NO: 142 - protein, 214 aa, synthetic construct
DIQMTQSPSTLSASVGDRVTITCRASQSISNWLAWYQQKPGKAPKVLIYKASSLESGVPSRFSGSGSGTEFTLTISSLQPDDFATYYCQQYNSFSWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - DNA, 642 bp, synthetic construct
  source 1..642: mol_type = other DNA
gacatccagatgacccagtctccttccaccctgtctgcatctgtaggagacagagtcaccatcacttgccgggccagtcagagtattagtaattggttggcctggtatcagcagaaaccagggaaagcccctaaggtcctgatctataaggcgtctagtttagaaagtggggtcccatcaaggttcagcggcagtggatctgggacagaattcactctcaccatcagcagcctgcagcctgatgattttgcaacttattactgccaacagtataatagtttttcgtggacgttcggccaagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 144 - protein, 124 aa, synthetic construct
QVQLVESGGGVVQPGRSLRLSCTASGFTFSNYAMHWVRQAPGKGLEWVAVISYDGSNKYCADSVKGRFTISRDNSKNTLYLQMNSLRAEDTAVYYCARGPLARRHYYYYGMDVWGQGTTVTVSS

SEQ ID NO: 145 - DNA, 373 bp, synthetic construct
  source 1..373: mol_type = other DNA
caggtgcagctggtggagtctgggggaggcgtggtccagcctgggaggtccctgagactctcctgtacagcctctggattcaccttcagtaactatgccatgcactgggtccgccaggctccaggcaagggactggagtgggtggcagttatatcatatgatggaagtaataaatactgtgcagactccgtgaagggccgattcaccatctccagagacaattccaagaacacgctgtatctgcaaatgaacagcctgagagctgaggacacggctgtgtattactgtgcgagaggaccactggcacgacgccactactactactacggtatggacgtctggggccaagggaccacggtcaccgtctcctcag

SEQ ID NO: 146 - protein, 451 aa, synthetic construct
QVQLVESGGGVVQPGRSLRLSCTASGFTFSNYAMHWVRQAPGKGLEWVAVISYDGSNKYCADSVKGRFTISRDNSKNTLYLQMNSLRAEDTAVYYCARGPLARRHYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 147 - DNA, 1353 bp, synthetic construct
  source 1..1353: mol_type = other DNA
caggtgcagctggtggagtctgggggaggcgtggtccagcctgggaggtccctgagactctcctgtacagcctctggattcaccttcagtaactatgccatgcactgggtccgccaggctccaggcaagggactggagtgggtggcagttatatcatatgatggaagtaataaatactgtgcagactccgtgaagggccgattcaccatctccagagacaattccaagaacacgctgtatctgcaaatgaacagcctgagagctgaggacacggctgtgtattactgtgcgagaggaccactggcacgacgccactactactactacggtatggacgtctggggccaagggaccacggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 148 - protein, 107 aa, synthetic construct
DIQMTQSPSSLSASVGDRVTITCRASQGIRNDLGWYQQKPGKAPKRLIYGASSLQSGVPSRFSGSGSGTEFTLTISSLQPEDFATYYCLQHNSYPRTFGQGTKVEIK

SEQ ID NO: 149 - DNA, 322 bp, synthetic construct
  source 1..322: mol_type = other DNA
gacatccagatgacccagtctccatcctccctgtctgcatctgtaggagacagagtcaccatcacttgccgggcaagtcagggcattagaaatgatttaggctggtatcagcagaagccagggaaagcccctaagcgcctgatctatggtgcatccagtttgcaaagtggagtcccatcaaggttcagcggcagtggatctgggacagaattcactctcacaatcagcagcctgcagcctgaagattttgcaacttattactgtctacagcataatagttaccctcggacgttcggccaagggaccaaggtggaaatcaaac

SEQ ID NO: 150 - protein, 214 aa, synthetic construct
DIQMTQSPSSLSASVGDRVTITCRASQGIRNDLGWYQQKPGKAPKRLIYGASSLQSGVPSRFSGSGSGTEFTLTISSLQPEDFATYYCLQHNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - DNA, 642 bp, synthetic construct
  source 1..642: mol_type = other DNA
gacatccagatgacccagtctccatcctccctgtctgcatctgtaggagacagagtcaccatcacttgccgggcaagtcagggcattagaaatgatttaggctggtatcagcagaagccagggaaagcccctaagcgcctgatctatggtgcatccagtttgcaaagtggagtcccatcaaggttcagcggcagtggatctgggacagaattcactctcacaatcagcagcctgcagcctgaagattttgcaacttattactgtctacagcataatagttaccctcggacgttcggccaagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 152 - protein, 121 aa, synthetic construct; identical to SEQ ID NO: 112

SEQ ID NO: 153 - DNA, 364 bp, synthetic construct
  source 1..364: mol_type = other DNA
caggtgcagctggtggagtctgggggaggcgtggtccagcctgggaggtccctgagactctcctgtgcagcgtctggattcaccttctatagctatggcatgcactgggtccgccaggctccaggcaaggggctggagtgggtggcagttatatggtatggcggaactaataaatactatgcagactccgtgaagggccgattcaccatctccagagacaattccaagaatacgctgtatctgcaaatgaacagcctgacagccgaagacacggctgtgtattactgtgcgagagggcaattggaacgccgacggggcggtttgaatatctggggccaagggacaatggtcaccgtctcttcag

SEQ ID NO: 154 - protein, 448 aa, synthetic construct; identical to SEQ ID NO: 114

SEQ ID NO: 155 - DNA, 1344 bp, synthetic construct
  source 1..1344: mol_type = other DNA
caggtgcagctggtggagtctgggggaggcgtggtccagcctgggaggtccctgagactctcctgtgcagcgtctggattcaccttctatagctatggcatgcactgggtccgccaggctccaggcaaggggctggagtgggtggcagttatatggtatggcggaactaataaatactatgcagactccgtgaagggccgattcaccatctccagagacaattccaagaatacgctgtatctgcaaatgaacagcctgacagccgaagacacggctgtgtattactgtgcgagagggcaattggaacgccgacggggcggtttgaatatctggggccaagggacaatggtcaccgtctcttcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 156 - protein, 107 aa, synthetic construct
DIQMTQSPSSLSASVGDRVTITCRASQGVRDDLGWYQQKPGKAPKRLIYAASNLQSGVPSRFSGSGSGTEFTLTIGSLQPEDFATYYCLQHNSYPFTFGPGTKVDIK

SEQ ID NO: 157 - DNA, 322 bp, synthetic construct
  source 1..322: mol_type = other DNA
gacatccagatgacccagtctccatcctccctgtctgcatctgtaggagacagagtcaccatcacttgccgggcaagtcagggcgttagagatgatttaggttggtatcagcagaaaccagggaaagcccctaagcgcctgatctatgctgcatccaatttgcaaagtggggtcccatcaaggttcagcggcagtggatctgggacagaattcactctcacaatcggcagcctgcagcctgaagattttgcaacttattactgtctacagcataatagttatccattcactttcggccctgggaccaaagtggatatcaaac

SEQ ID NO: 158 - protein, 214 aa, synthetic construct
DIQMTQSPSSLSASVGDRVTITCRASQGVRDDLGWYQQKPGKAPKRLIYAASNLQSGVPSRFSGSGSGTEFTLTIGSLQPEDFATYYCLQHNSYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 159 - DNA, 642 bp, synthetic construct
  source 1..642: mol_type = other DNA
gacatccagatgacccagtctccatcctccctgtctgcatctgtaggagacagagtcaccatcacttgccgggcaagtcagggcgttagagatgatttaggttggtatcagcagaaaccagggaaagcccctaagcgcctgatctatgctgcatccaatttgcaaagtggggtcccatcaaggttcagcggcagtggatctgggacagaattcactctcacaatcggcagcctgcagcctgaagattttgcaacttattactgtctacagcataatagttatccattcactttcggccctgggaccaaagtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 160 - protein, 130 aa, synthetic construct
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCARGRGYYGSGSHYRGYYYYGLDVWGQGTTVTVSS

SEQ ID NO: 161 - DNA, 391 bp, synthetic construct
  source 1..391: mol_type = other DNA
caggtgcagctggtgcagtctggggctgaggtgaagaagcctggggcctcagtgaaggtctcctgcaaggcttctggatacaccttcaccagttatgatatcaactgggtgcgacaggccactggacaagggcttgagtggatgggatggatgaaccctaacagtggtaacacaggctatgcacagaagttccagggcagagtcaccatgaccaggaacacctccataagcacagcctacatggagctgagcagcctgagatctgaggacacggccgtgtattactgtgcgagaggccgggggtactatggttcggggagccattataggggctactactactacggtttggacgtctggggccaagggaccacggtcaccgtctcctcag

SEQ ID NO: 162 - protein, 457 aa, synthetic construct
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCARGRGYYGSGSHYRGYYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 163 - DNA, 1371 bp, synthetic construct
  source 1..1371: mol_type = other DNA
caggtgcagctggtgcagtctggggctgaggtgaagaagcctggggcctcagtgaaggtctcctgcaaggcttctggatacaccttcaccagttatgatatcaactgggtgcgacaggccactggacaagggcttgagtggatgggatggatgaaccctaacagtggtaacacaggctatgcacagaagttccagggcagagtcaccatgaccaggaacacctccataagcacagcctacatggagctgagcagcctgagatctgaggacacggccgtgtattactgtgcgagaggccgggggtactatggttcggggagccattataggggctactactactacggtttggacgtctggggccaagggaccacggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 164 - protein, 107 aa, synthetic construct
DIQMTQSPSTLSASVGDRVTITCRASQSISSWLAWYQQKPGKAPKVLIYKASSLKSGVPSRFSGSGSGTEFTLTVSSLQPDDFATYYCQQYNSYSWTFGQGTKVEIK

SEQ ID NO: 165 - DNA, 322 bp, synthetic construct
  source 1..322: mol_type = other DNA
gacatccagatgacccagtctccttccaccctgtctgcatctgtaggagacagagtcaccatcacttgccgggccagtcagagtattagtagctggttggcctggtatcagcagaaaccagggaaagcccctaaggtcctgatctataaggcgtctagtttaaaaagtggggtcccatcaaggttcagcggcagtggatctgggacagaattcactctcaccgtcagcagcctgcagcctgatgattttgcaacttattactgccaacagtataatagttattcgtggacgttcggccaagggaccaaggtggaaatcaaac

SEQ ID NO: 166 - protein, 214 aa, synthetic construct
DIQMTQSPSTLSASVGDRVTITCRASQSISSWLAWYQQKPGKAPKVLIYKASSLKSGVPSRFSGSGSGTEFTLTVSSLQPDDFATYYCQQYNSYSWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 167 - DNA, 642 bp, synthetic construct
  source 1..642: mol_type = other DNA
gacatccagatgacccagtctccttccaccctgtctgcatctgtaggagacagagtcaccatcacttgccgggccagtcagagtattagtagctggttggcctggtatcagcagaaaccagggaaagcccctaaggtcctgatctataaggcgtctagtttaaaaagtggggtcccatcaaggttcagcggcagtggatctgggacagaattcactctcaccgtcagcagcctgcagcctgatgattttgcaacttattactgccaacagtataatagttattcgtggacgttcggccaagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 168 - protein, 129 aa, synthetic construct
QVQLQESGPGLVKPSQTLSLTCTVSGGSISSGRYYWSWIRQLPGKGLEWIGYISYSGNPFYSPSLKSRVTISVDTSENQFSLKLSSVTAADTAVYYCARDRRIPMLRGVKGYYYGMDVWGQGTTVTVSS

SEQ ID NO: 169 - DNA, 388 bp, synthetic construct
  source 1..388: mol_type = other DNA
caggtgcagctgcaggagtcgggcccaggactggtgaagccttcacagaccctgtccctcacctgcactgtctctggtggctccatcagcagtggtagatactactggagctggatccgccagctcccagggaagggcctggagtggattgggtacatctcatacagtgggaaccccttctacagcccgtccctcaagagtcgagttaccatatcagtagacacgtctgagaaccaattctccctgaagctgagctctgtgactgccgcggacacggccgtgtattactgtgcgagagatcgacgtattcctatgcttcggggagttaaaggctactactacggtatggacgtctggggccaagggaccacggtcaccgtctcctcag

SEQ ID NO: 170 - protein, 456 aa, synthetic construct
QVQLQESGPGLVKPSQTLSLTCTVSGGSISSGRYYWSWIRQLPGKGLEWIGYISYSGNPFYSPSLKSRVTISVDTSENQFSLKLSSVTAADTAVYYCARDRRIPMLRGVKGYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 171 - DNA, 1368 bp, synthetic construct
  source 1..1368: mol_type = other DNA
caggtgcagctgcaggagtcgggcccaggactggtgaagccttcacagaccctgtccctcacctgcactgtctctggtggctccatcagcagtggtagatactactggagctggatccgccagctcccagggaagggcctggagtggattgggtacatctcatacagtgggaaccccttctacagcccgtccctcaagagtcgagttaccatatcagtagacacgtctgagaaccaattctccctgaagctgagctctgtgactgccgcggacacggccgtgtattactgtgcgagagatcgacgtattcctatgcttcggggagttaaaggctactactacggtatggacgtctggggccaagggaccacggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 172 - protein, 112 aa, synthetic construct
DIVMTQSPLSLPVIPGEPASISCRSSQSLLHSNGYNYLDWYLQKPGQSPHLLIYLGSNRASGVPDRFSGSGSGTDFTLKISRVEAEDVGVYYCMQALQTPWTFGQGTKVEIK

SEQ ID NO: 173 - DNA, 337 bp, synthetic construct
  source 1..337: mol_type = other DNA
gatattgtgatgactcagtctccactctccctgcccgtcatccctggagagccggcctccatctcctgcaggtctagtcagagcctcctgcatagtaatggatacaactatttggattggtacctgcagaagccagggcagtctccacacctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgtggacgttcggccaagggaccaaggtggaaatcaaac

SEQ ID NO: 174 - protein, 219 aa, synthetic construct
DIVMTQSPLSLPVIPGEPASISCRSSQSLLHSNGYNYLDWYLQKPGQSPHLLIYLGSNRASGVPDRFSGSGSGTDFTLKISRVEAEDVGVYYCMQALQTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - DNA, 657 bp, synthetic construct
  source 1..657: mol_type = other DNA
gatattgtgatgactcagtctccactctccctgcccgtcatccctggagagccggcctccatctcctgcaggtctagtcagagcctcctgcatagtaatggatacaactatttggattggtacctgcagaagccagggcagtctccacacctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgtggacgttcggccaagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 176 - protein, 124 aa, synthetic construct
EVQLVESGGGLVQPGGSLRLSCAASGFTFSNYAMSWVRQAPRKGLEWVSVISGSGGRTNYAGSVKGRFSITRDNSKNTLYLQMNSLRAEDTAVYYCVKGQLERRYYYYYGMDVWGQGITVTVSS

SEQ ID NO: 177 - DNA, 373 bp, synthetic construct
  source 1..373: mol_type = other DNA
gaggtgcagctggtggagtctgggggaggcttggtacagcctggggggtccctgagactctcctgtgcagcctctggattcacctttagcaactatgccatgagctgggtccgccaggctccaaggaaggggctggagtgggtctcagttattagtggtagtggtggtagaacaaactacgcaggctccgtgaagggccggttctccatcaccagagacaattccaagaacacgctgtatctgcaaatgaacagcctgagagccgaggacacggccgtatattactgtgtgaaaggccaattggaacgacggtactactactattacggtatggacgtctggggccaagggattacggtcaccgtctcctcag

SEQ ID NO: 178 - protein, 451 aa, synthetic construct
EVQLVESGGGLVQPGGSLRLSCAASGFTFSNYAMSWVRQAPRKGLEWVSVISGSGGRTNYAGSVKGRFSITRDNSKNTLYLQMNSLRAEDTAVYYCVKGQLERRYYYYYGMDVWGQGITVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 179 - DNA, 1353 bp, synthetic construct
  source 1..1353: mol_type = other DNA
gaggtgcagctggtggagtctgggggaggcttggtacagcctggggggtccctgagactctcctgtgcagcctctggattcacctttagcaactatgccatgagctgggtccgccaggctccaaggaaggggctggagtgggtctcagttattagtggtagtggtggtagaacaaactacgcaggctccgtgaagggccggttctccatcaccagagacaattccaagaacacgctgtatctgcaaatgaacagcctgagagccgaggacacggccgtatattactgtgtgaaaggccaattggaacgacggtactactactattacggtatggacgtctggggccaagggattacggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 180 - protein, 107 aa, synthetic construct
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPLTFGGGTKVEIK

SEQ ID NO: 181 - DNA, 322 bp, synthetic construct
  source 1..322: mol_type = other DNA
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgctcactttcggcggagggaccaaggtggagatcaaac

SEQ ID NO: 182 - protein, 214 aa, synthetic construct
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 183 - DNA, 642 bp, synthetic construct
  source 1..642: mol_type = other DNA
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgctcactttcggcggagggaccaaggtggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 184 - protein, 124 aa, synthetic construct
EVQLVESGGGLVKPGGSLRLSCAASGFTFSSYSMNWVRQAPGKGLEWVSSISSSRRYIYYADSVKGRFTISRDNAKNSLYLQMNSLRAEDTAVYYCARAYGQVAPYYYYGMDVWGQGTTVTVSS

SEQ ID NO: 185 - DNA, 373 bp, synthetic construct
  source 1..373: mol_type = other DNA
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagacgttacatatactacgcagactcagtgaagggccgattcaccatctccagagacaacgccaagaactcactgtatctgcaaatgaacagcctgagagccgaggacacagctgtgtattactgtgcgagagcctatgggcaggtcgccccctactactactacggtatggacgtctggggccaagggaccacggtcaccgtctcctcag

SEQ ID NO: 186 - protein, 451 aa, synthetic construct
EVQLVESGGGLVKPGGSLRLSCAASGFTFSSYSMNWVRQAPGKGLEWVSSISSSRRYIYYADSVKGRFTISRDNAKNSLYLQMNSLRAEDTAVYYCARAYGQVAPYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 187 - DNA, 1353 bp, synthetic construct
  source 1..1353: mol_type = other DNA
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagacgttacatatactacgcagactcagtgaagggccgattcaccatctccagagacaacgccaagaactcactgtatctgcaaatgaacagcctgagagccgaggacacagctgtgtattactgtgcgagagcctatgggcaggtcgccccctactactactacggtatggacgtctggggccaagggaccacggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 188 - protein, 107 aa, synthetic construct [sequence omitted]

SEQ ID NO: 189 - DNA, 322 bp, synthetic construct
  source 1..322: mol_type = other DNA
gccatccagttgacccagtctccatcctccctgtctgcatctgtaggagacagagtcaccatcacttgccgggcaagtcagggcattagcagtgctttagcctggtatcagcagaaaccagggaaagctcctaaactcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgctcactttcggcggagggaccaaggtggagatcaaac

SEQ ID NO: 190 - protein, 214 aa, synthetic construct
AIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 191 - DNA, 642 bp, synthetic construct
  source 1..642: mol_type = other DNA
gccatccagttgacccagtctccatcctccctgtctgcatctgtaggagacagagtcaccatcacttgccgggcaagtcagggcattagcagtgctttagcctggtatcagcagaaaccagggaaagctcctaaactcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccgctcactttcggcggagggaccaaggtggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 192 - protein, 130 aa, synthetic construct
QVQLVESGGGVVQPGRSLRLSCEASGFTLSSYGMHWVRQAPGKGLEWVAVIWNDGSNKYSADSVKGRFTISRDNSKNTLYLQMNRLRAEDTAVYYCARGGGYYGSGSHYRGYYYYGMDVWGQGTTVTVSS

SEQ ID NO: 193 - DNA, 391 bp, synthetic construct
  source 1..391: mol_type = other DNA
caggtgcagctggtggagtctgggggaggcgtggtccagcctgggaggtccctgagactctcctgtgaagcgtctggattcaccctcagtagctatggcatgcactgggtccgccaggctccaggcaaggggctggagtgggtggcagttatatggaatgatggaagtaataaatattctgcagactccgtgaagggccgattcaccatctccagagacaattccaagaacaccctgtatctgcaaatgaacagactgagagccgaggacacggctgtgtattattgtgcgagagggggtggttactatggttcggggagtcattatagaggatactattactacggtatggacgtctggggccaagggaccacggtcaccgtctcctcag

SEQ ID NO: 194 - protein, 457 aa, synthetic construct
QVQLVESGGGVVQPGRSLRLSCEASGFTLSSYGMHWVRQAPGKGLEWVAVIWNDGSNKYSADSVKGRFTISRDNSKNTLYLQMNRLRAEDTAVYYCARGGGYYGSGSHYRGYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 195 - DNA, 1371 bp, synthetic construct
  source 1..1371: mol_type = other DNA
caggtgcagctggtggagtctgggggaggcgtggtccagcctgggaggtccctgagactctcctgtgaagcgtctggattcaccctcagtagctatggcatgcactgggtccgccaggctccaggcaaggggctggagtgggtggcagttatatggaatgatggaagtaataaatattctgcagactccgtgaagggccgattcaccatctccagagacaattccaagaacaccctgtatctgcaaatgaacagactgagagccgaggacacggctgtgtattattgtgcgagagggggtggttactatggttcggggagtcattatagaggatactattactacggtatggacgtctggggccaagggaccacggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 196 - protein, 107 aa, synthetic construct
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPWTFGQGTKVEIK

SEQ ID NO: 197 - DNA, 322 bp, synthetic construct
  source 1..322: mol_type = other DNA
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgtggacgttcggccaagggaccaaggtggaaatcaaac

SEQ ID NO: 198 - protein, 214 aa, synthetic construct
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 199 - DNA, 642 bp, synthetic construct
  source 1..642: mol_type = other DNA
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgtggacgttcggccaagggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 200 - protein, 122 aa, synthetic construct
QVQLVESGGGVVQPGRSLRLSCAASGFTFSSYGMHWVRQAPGKGLEWVTVILYGGSNKYYADSVKGRFTISRDNSKNTLYLQMNSLRAEDTAVYYCARGMLTYYYSSGFDYWGQGTLVTVSS

SEQ ID NO: 201 - DNA, 367 bp, synthetic construct
  source 1..367: mol_type = other DNA
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtaacagttatattgtatggtggaagtaataaatactatgcagactccgtgaagggccgtttcaccatctccagagacaattccaagaacacgctgtatctgcaaatgaacagcctcagagccgaggacacggctgtgtattactgtgcgagagggatgttaacgtattactatagttcggggtttgactactggggccagggaaccctggtcaccgtctcctcag

SEQ ID NO: 202 - protein, 449 aa, synthetic construct
QVQLVESGGGVVQPGRSLRLSCAASGFTFSSYGMHWVRQAPGKGLEWVTVILYGGSNKYYADSVKGRFTISRDNSKNTLYLQMNSLRAEDTAVYYCARGMLTYYYSSGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 203 - DNA, 1347 bp, synthetic construct
  source 1..1347: mol_type = other DNA
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtaacagttatattgtatggtggaagtaataaatactatgcagactccgtgaagggccgtttcaccatctccagagacaattccaagaacacgctgtatctgcaaatgaacagcctcagagccgaggacacggctgtgtattactgtgcgagagggatgttaacgtattactatagttcggggtttgactactggggccagggaaccctggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 204 - protein, 112 aa, synthetic construct
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPFTFGPGTKVDIK

SEQ ID NO: 205 - DNA, 337 bp, synthetic construct
  source 1..337: mol_type = other DNA
gatgttgtgatgactcagtctccactctccctgcccgtcacccttggacagccggcctccatctcctgcaggtctagtcaaagcctcgtatacagtgatggaaacacctacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggccattcactttcggccctgggaccaaagtggatatcaaac

SEQ ID NO: 206 - protein, 219 aa, synthetic construct
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 207 - DNA, 657 bp, synthetic construct
  source 1..657: mol_type = other DNA
gatgttgtgatgactcagtctccactctccctgcccgtcacccttggacagccggcctccatctcctgcaggtctagtcaaagcctcgtatacagtgatggaaacacctacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggccattcactttcggccctgggaccaaagtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 208 - protein, 130 aa, synthetic construct
QVQLVESGGGVVQPGRSLRLSCAASGFTFNNYGMHWVRQAPGKGLEWVAVIWNDGSNKYYGDSVKGRFTISRDNSKNTLYLQMNSLRAEDTAVYYCARGGGYYGSGKHYRGYYYNGMGVWGQGTPVTVSS

SEQ ID NO: 209 - DNA, 391 bp, synthetic construct
  source 1..391: mol_type = other DNA
caggtgcagttggtggagtctgggggaggcgtggtccagcctgggaggtccctgagactctcctgtgcagcgtctggattcaccttcaataactatggcatgcactgggtccgccaggctccaggcaaggggctggagtgggtggcagttatatggaatgatggaagtaataaatactatggagactccgtgaagggccgattcaccatctccagagacaattccaagaacacgctgtatctgcaaatgaacagcctgagagccgaggacacggctgtgtattactgtgcgagagggggtggttactatggttcggggaaacattatagaggctattactataacggtatgggcgtctggggccaagggaccccggtcaccgtctcctcag

SEQ ID NO: 210 - protein, 457 aa, synthetic construct
QVQLVESGGGVVQPGRSLRLSCAASGFTFNNYGMHWVRQAPGKGLEWVAVIWNDGSNKYYGDSVKGRFTISRDNSKNTLYLQMNSLRAEDTAVYYCARGGGYYGSGKHYRGYYYNGMGVWGQGTPVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 211 - DNA, 1371 bp, synthetic construct
  source 1..1371: mol_type = other DNA
caggtgcagttggtggagtctgggggaggcgtggtccagcctgggaggtccctgagactctcctgtgcagcgtctggattcaccttcaataactatggcatgcactgggtccgccaggctccaggcaaggggctggagtgggtggcagttatatggaatgatggaagtaataaatactatggagactccgtgaagggccgattcaccatctccagagacaattccaagaacacgctgtatctgcaaatgaacagcctgagagccgaggacacggctgtgtattactgtgcgagagggggtggttactatggttcggggaaacattatagaggctattactataacggtatgggcgtctggggccaagggaccccggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 212 - protein, 107 aa, synthetic construct
EIVMTQSPATLSVSPGERATLSCRASQSVSSNLAWYQQKPGQAPRLLIYGASTRATGIPARFSGSGSGTEVTLTISSLQSEDFTIYYCQQYNNWPWTFGQGTKVDIK

SEQ ID NO: 213 - DNA, 322 bp, synthetic construct
  source 1..322: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagaggtcactctcaccatcagcagcctgcagtctgaagattttacaatttattactgtcagcagtataataactggccgtggacgttcggccaagggaccaaggtggatatcaaac

SEQ ID NO: 214 - protein, 214 aa, synthetic construct
EIVMTQSPATLSVSPGERATLSCRASQSVSSNLAWYQQKPGQAPRLLIYGASTRATGIPARFSGSGSGTEVTLTISSLQSEDFTIYYCQQYNNWPWTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 215 - DNA, 642 bp, synthetic construct
  source 1..642: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagaggtcactctcaccatcagcagcctgcagtctgaagattttacaatttattactgtcagcagtataataactggccgtggacgttcggccaagggaccaaggtggatatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 216 - protein, 128 aa, synthetic construct
QVQLQQWGAGLLKPSETLSLTCAVYGGSFSGYYWSWIRQPPGKGLEWIGKINHSGSTNYKPSLKSRVNISVDTSKNQFSLKLTSVTAADTAVYYCAREASMLRGVKGYRFYLYAMDVWGQGTSVTVSS

SEQ ID NO: 217 - DNA, 385 bp, synthetic construct
  source 1..385: mol_type = other DNA
caggtgcagctacaacagtggggcgcaggactgttgaagccttcggagaccctgtccctcacctgcgctgtctatggtgggtccttcagtggttactactggagttggatccgccagcccccagggaaggggctggagtggattgggaaaatcaatcatagtggaagcaccaactacaaaccgtccctcaagagtcgagtcaacatatcagtagacacgtccaagaaccagttctccctgaagctgacctctgtgaccgccgcggacacggctgtgtattactgtgcgagagaggcctctatgcttcggggagttaaggggtatcgcttctacttatacgcaatggacgtctggggccaagggacctcggtcaccgtctcctcag

SEQ ID NO: 218 - protein, 851 aa, synthetic construct
QVQLQQWGAGLLKPSETLSLTCAVYGGSFSGYYWSWIRQPPGKGLEWIGKINHSGSTNYKPSLKSRVNISVDTSKNQFSLKLTSVTAADTAVYYCAREASMLRGVKGYRFYLYAMDVWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKDSVKGRFTISRDNSKNTLYLQMNSLRAEDTAVYYCARGGGYYGSGKYYRGYYYNGMDVWGQGTPVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 219 - DNA, 1365 bp, synthetic construct
  source 1..1365: mol_type = other DNA
caggtgcagctacaacagtggggcgcaggactgttgaagccttcggagaccctgtccctcacctgcgctgtctatggtgggtccttcagtggttactactggagttggatccgccagcccccagggaaggggctggagtggattgggaaaatcaatcatagtggaagcaccaactacaaaccgtccctcaagagtcgagtcaacatatcagtagacacgtccaagaaccagttctccctgaagctgacctctgtgaccgccgcggacacggctgtgtattactgtgcgagagaggcctctatgcttcggggagttaaggggtatcgcttctacttatacgcaatggacgtctggggccaagggacctcggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 220 - protein, 108 aa, synthetic construct
SSELTQDPAVSVALGQTVRITCQGDSLRSYYASWYQQKPGQTPLLVIYGKNNRPSGIPDRFSGSSSGNTASLTITGAQAEDEADYYCNSRDSSGNHVVFGGGTKLTVL

SEQ ID NO: 221 - DNA, 325 bp, synthetic construct
  source 1..325: mol_type = other DNA
tcttctgagctgactcaggaccctgctgtgtctgtggccttgggacagacagtcaggatcacatgccaaggagacagcctcagaagctattatgcaagctggtaccagcagaagccaggacagacccctttacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccatgtggtgttcggcggagggaccaagctgaccgtcctag

SEQ ID NO: 222 - protein, 214 aa, synthetic construct
SSELTQDPAVSVALGQTVRITCQGDSLRSYYASWYQQKPGQTPLLVIYGKNNRPSGIPDRFSGSSSGNTASLTITGAQAEDEADYYCNSRDSSGNHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 223 - DNA, 642 bp, synthetic construct
  source 1..642: mol_type = other DNA
tcttctgagctgactcaggaccctgctgtgtctgtggccttgggacagacagtcaggatcacatgccaaggagacagcctcagaagctattatgcaagctggtaccagcagaagccaggacagacccctttacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccatgtggtgttcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 224 - protein, 124 aa, synthetic construct
EVQLVESGGDLVQPGGSLRLSCAASGFTFYSYAMNWVRQAPGKGLKWVSGISGSGDITYYADSVKGRFTISRDNSKNTLYLQMHSLRAEDTAVYYCAKGQLERRYYKYYGMDVWGQGTTVTVSS

SEQ ID NO: 225 - DNA, 373 bp, synthetic construct
  source 1..373: mol_type = other DNA
gaggtgcagctggtggagtctgggggagacttagtacagcctggggggtccctgagactctcttgtgcagcctctggattcaccttttacagctatgccatgaactgggtccgccaggctccagggaaggggctaaaatgggtctcaggtattagtggtagtggtgatatcacatactacgcagactccgtgaagggccggttcaccatctccagagacaattccaagaacacgctgtatctgcaaatgcacagcctgagagccgaagacacggccgtatattactgtgcgaaaggccaactggaacgacggtactacaagtactacggtatggacgtctggggccaagggaccacggtcaccgtctcctcag

SEQ ID NO: 226 - protein, 451 aa, synthetic construct
EVQLVESGGDLVQPGGSLRLSCAASGFTFYSYAMNWVRQAPGKGLKWVSGISGSGDITYYADSVKGRFTISRDNSKNTLYLQMHSLRAEDTAVYYCAKGQLERRYYKYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 227 - DNA, 1353 bp, synthetic construct
  source 1..1353: mol_type = other DNA
gaggtgcagctggtggagtctgggggagacttagtacagcctggggggtccctgagactctcttgtgcagcctctggattcaccttttacagctatgccatgaactgggtccgccaggctccagggaaggggctaaaatgggtctcaggtattagtggtagtggtgatatcacatactacgcagactccgtgaagggccggttcaccatctccagagacaattccaagaacacgctgtatctgcaaatgcacagcctgagagccgaagacacggccgtatattactgtgcgaaaggccaactggaacgacggtactacaagtactacggtatggacgtctggggccaagggaccacggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 228 - protein, 107 aa, synthetic construct; identical to SEQ ID NO: 46

SEQ ID NO: 229 - DNA, 322 bp, synthetic construct
  source 1..322: mol_type = other DNA
gacatccagatgacccagtctccatcctccctgtctgcatctgtaggagacagagtcaccatcacttgccgggcaaatcagggcattagaaatgatttaggctggtatcagcagaaaccagggaaagcccctaagcgcctgatctatggtgcatccagtttgaaaagtggggtcccatcaaggttcagcggcagtggatctgggacagaattcactctcacaatcagcagcctgcagcctgaagattttgcaacctactattgtctacagcagaatagttacccgctcactttcggcggagggaccaaggtggagatcaaac

SEQ ID NO: 230 - protein, 214 aa, synthetic construct; identical to SEQ ID NO: 48

SEQ ID NO: 231 - DNA, 642 bp, synthetic construct
  source 1..642: mol_type = other DNA
gacatccagatgacccagtctccatcctccctgtctgcatctgtaggagacagagtcaccatcacttgccgggcaaatcagggcattagaaatgatttaggctggtatcagcagaaaccagggaaagcccctaagcgcctgatctatggtgcatccagtttgaaaagtggggtcccatcaaggttcagcggcagtggatctgggacagaattcactctcacaatcagcagcctgcagcctgaagattttgcaacctactattgtctacagcagaatagttacccgctcactttcggcggagggaccaaggtggagatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 232 - protein, 115 aa, synthetic construct
AVQLVESGGGLVQPGGSLKLSCAASGFTFSGSTMHWVRQASGRGLEWVGRIRSKADSYATAYSASVKGRFTISRDDSKKMAYLQMNSLKTEDMAVYYCTRSGDYWGQGTLVTVSS

SEQ ID NO: 233 - DNA, 345 bp, synthetic construct
  source 1..345: mol_type = other DNA
gcggtgcagctggtggagtccgggggaggcttggtccagcctggggggtccctgaaactctcctgtgcagcctctggattcaccttcagtggctctactatgcactggtccgccaggcttccgggagagggctggagtgggttggccgtattagaagcaaagctgacagttacgcgacagcatatagtgcgtcggtgaaaggcaggttcaccatctccagagatgattcaaagaaaatggcgtatctgcaaatgaacagcctgaaaaccgaggacatggccgtgtattattgtactagaagcggggactactggggccagggaaccctggtcaccgtctcctcag

SEQ ID NO: 234 - protein, 442 aa, synthetic construct
AVQLVESGGGLVQPGGSLKLSCAASGFTFSGSTMHWVRQASGRGLEWVGRIRSKADSYATAYSASVKGRFTISRDDSKKMAYLQMNSLKTEDMAVYYCTRSGDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 235 - DNA, 1325 bp, synthetic construct
  source 1..1325: mol_type = other DNA
gcggtgcagctggtggagtccgggggaggcttggtccagcctggggggtccctgaaactctcctgtgcagcctctggattcaccttcagtggctctactatgcactggtccgccaggcttccgggagagggctggagtgggttggccgtattagaagcaaagctgacagttacgcgacagcatatagtgcgtcggtgaaaggcaggttcaccatctccagagatgattcaaagaaaatggcgtatctgcaaatgaacagcctgaaaaccgaggacatggccgtgtattattgtactagaagcggggactactggggccagggaaccctggtcaccgtctcctcagccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 236 - protein, 111 aa, synthetic construct
QSVLTQPPSVSAAPGQKVTISCSGSSSNIGKNYVAWYQQLPGTAPKLLIYDTNKRPSGIPDRFSGSKSGTSATLDITGLQTGDEADYYCGTWDISLSVGWLFGGGTKVTVL

SEQ ID NO: 237 - DNA, 334 bp, synthetic construct
  source 1..334: mol_type = other DNA
cagtctgtgttgacgcagccgccctcagtgtctgcggccccaggacagaaggtcaccatctcctgttctggaagcagctccaacattggaaaaaattatgtagcctggtaccagcaactcccaggaacagcccccaaactcctcatttatgacactaataagcgaccctcagggattcctgaccgattctctggctccaagtctggcacgtcagccaccctggacatcaccggactccagactggggacgaggccgattattactgcggaacatgggatatcagcctgagtgttggttggctgttcggcggagggaccaaggtgaccgtcctag

SEQ ID NO: 238 - protein, 217 aa, synthetic construct
QSVLTQPPSVSAAPGQKVTISCSGSSSNIGKNYVAWYQQLPGTAPKLLIYDTNKRPSGIPDRFSGSKSGTSATLDITGLQTGDEADYYCGTWDISLSVGWLFGGGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 239 - DNA, 652 bp, synthetic construct
  source 1..652: mol_type = other DNA
cagtctgtgttgacgcagccgccctcagtgtctgcggccccaggacagaaggtcaccatctcctgttctggaagcagctccaacattggaaaaaattatgtagcctggtaccagcaactcccaggaacagcccccaaactcctcatttatgacactaataagcgaccctcagggattcctgaccgattctctggctccaagtctggcacgtcagccaccctggacatcaccggactccagactggggacgaggccgattattactgcggaacatgggatatcagcctgagtgttggttggctgttcggcggagggaccaaggtgaccgtcctaggccagcctaaggccgctccttccgttacactgttccctccatcctccgaggaactgcaggccaacaaggctaccctcgtgtgcctgatctccgacttttaccctggcgctgtgaccgtggcctggaaggctgatagttctcctgtgaaggccggcgtggaaaccaccacaccttccaagcagtccaacaacaaatacgccgcctcctcctacctgtctctgacccctgaacagtggaagtcccacaagtcctacagctgccaagtgacccacgagggctccaccgtggaaaagacagtggctcctaccgagtgctcca

SEQ ID NO: 240 - DNA, 990 bp, Homo sapiens
  source 1..990: mol_type = other DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 241 - protein, 330 aa, Homo sapiens [sequence omitted]

SEQ ID NO: 242 - DNA, 990 bp, Homo sapiens
  source 1..990: mol_type = other DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 243 - protein, 330 aa, Homo sapiens [sequence omitted]

SEQ ID NO: 244 - DNA, 990 bp, Homo sapiens
  source 1..990: mol_type = other DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 245 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 246 - DNA, 990 bp, Homo sapiens
  source 1..990: mol_type = other DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacatcttctcatgctccgtgatgcatgaggctctgcacaaccactacacgcagaagagcctctccctgtctccgggtaaa

SEQ ID NO: 247 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPGK

SEQ ID NO: 248 - DNA, 990 bp, synthetic construct
  source 1..990: mol_type = other DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtggagcccaaatcttgtgacaaaactcacacatgcccaccgtgcccagcacctgaactcgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 249 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 250 - DNA, 978 bp, Homo sapiens
  source 1..978: mol_type = other DNA
gcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 251 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 252 - DNA, 978 bp, Homo sapiens
  source 1..978: mol_type = other DNA
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgacctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcatggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 253 - protein, 326 aa, Homo sapiens
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 254 - DNA, 978 bp, Homo sapiens
  source 1..978: mol_type = other DNA
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcagcttgggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 255 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 256 - DNA, 978 bp, Homo sapiens
  source 1..978: mol_type = other DNA
gcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatctccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaa

SEQ ID NO: 257 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 258 - DNA, 981 bp, Homo sapiens
  source 1..981: mol_type = other DNA
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 259 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 260 - DNA, 981 bp, Homo sapiens
  source 1..981: mol_type = other DNA
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccgtgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcgtgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacgcagaagagcctctccctgtctctgggtaaa

SEQ ID NO: 261 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 262 - DNA, 981 bp, Homo sapiens
  source 1..981: mol_type = other DNA
gcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctcaccgtggacaagagcaggtggcaggaggggaacgtcttctcatgctccgtgatgcatgaggctctgcacaaccactacacgcagaagagcctctccctgtctctgggtaaa

SEQ ID NO: 263 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 264 - DNA, 981 bp, Homo sapiens
  source 1..981: mol_type = other DNA
gcctccaccaagggcccatccgtcttccccctggcgccctgctccaggagcacctccgagagcacggccgccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcgcctgaatttgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcatcgatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggatccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 265 - DNA, 981 bp, Homo sapiens
  source 1..981: mol_type = other DNA
gcctccaccaagggacctagcgtgttccctctcgccccctgttccaggtccacaagcgagtccaccgctgccctcggctgtctggtgaaagactactttcccgagcccgtgaccgtctcctggaatagcggagccctgacctccggcgtgcacacatttcccgccgtgctgcagagcagcggactgtatagcctgagcagcgtggtgaccgtgcccagctccagcctcggcaccaaaacctacacctgcaacgtggaccacaagccctccaacaccaaggtggacaagcgggtggagagcaagtacggccccccttgccctccttgtcctgcccctgagttcgagggaggaccctccgtgttcctgtttccccccaaacccaaggacaccctgatgatctcccggacacccgaggtgacctgtgtggtcgtggacgtcagccaggaggaccccgaggtgcagttcaactggtatgtggacggcgtggaggtgcacaatgccaaaaccaagcccagggaggagcagttcaattccacctacagggtggtgagcgtgctgaccgtcctgcatcaggattggctgaacggcaaggagtacaagtgcaaggtgtccaacaagggactgcccagctccatcgagaagaccatcagcaaggctaagggccagccgagggagccccaggtgtataccctgcctcctagccaggaagagatgaccaagaaccaagtgtccctgacctgcctggtgaagggattctacccctccgacatcgccgtggagtgggagagcaatggccagcccgagaacaactacaaaacaacccctcccgtgctcgatagcgacggcagcttctttctctacagccggctgacagtggacaagagcaggtggcaggagggcaacgtgttctcctgttccgtgatgcacgaggccctgcacaatcactacacccagaagagcctctccctgtccctgggcaag

SEQ ID NO: 266 - DNA, 981 bp, Homo sapiens
  source 1..981: mol_type = other DNA
gccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccaggaggagatgaccaagaaccaggtgagcctgacctgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaag

SEQ ID NO: 267 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 268 - DNA, 981 bp, synthetic construct
  source 1..981: mol_type = other DNA
gcctccaccaagggcccatccgtcttccccctggcgccctgctccaggagcacctccgagagcacggccgccctgggctgcctggtcaaggactacttccccgaaccagtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcgcctccagttgcggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcatcgatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggatccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 269 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 270 - DNA, 321 bp, Homo sapiens
  source 1..321: mol_type = other DNA
cgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 271 - protein, 107 aa, Homo sapiens [sequence omitted]

SEQ ID NO: 272 - DNA, 321 bp, Homo sapiens
  source 1..321: mol_type = other DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggagagcaaggacagcacctacagcctcagcagcaccctgacgctgagcaaagcagactacgagaaacacaaagtctacgccggcgaagtcacccatcagggcctgagctcgcccgtcacaaagagcttcaacaggggagagtgt

SEQ ID NO: 273 - protein, 107 aa, Homo sapiens
RTVAAPSVFIFPPSDEQLKSGTASVVCLLNNFYPREAKVQWKVDNALQSGNSQESVTEQESKDSTYSLSSTLTLSKADYEKHKVYAGEVTHQGLSSPVTKSFNRGEC

SEQ ID NO: 274 - DNA, 321 bp, Homo sapiens
  source 1..321: mol_type = other DNA
cgaactgtggctgcaccatctgtcttcatcttcccgccatctgatgagcagttgaaatctggaactgcctctgttgtgtgcctgctgaataacttctatcccagagaggccaaagtacagcggaaggtggataacgccctccaatcgggtaactcccaggagagtgtcacagagcaggagagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 275 - protein, 107 aa, Homo sapiens
RTVAAPSVFIFPPSDEQLKSGTASVVCLLNNFYPREAKVQRKVDNALQSGNSQESVTEQESKDSTYSLSSTLTLSKADYEKHKVYACEVTHQGLSSPVTKSFNRGEC

SEQ ID NO: 276 - DNA, 321 bp, Homo sapiens
  source 1..321: mol_type = other DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 277 - protein, 107 aa, Homo sapiens
RTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 278 - DNA, 321 bp, Homo sapiens
  source 1..321: mol_type = other DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcaacaccctgacgctgagcaaagcagactacgagaaacacaaagtctacgcctgcgaagtcacccatcagggcctgagctcgcccgtcacaaagagcttcaacaggggagagtgc

SEQ ID NO: 279 - protein, 107 aa, Homo sapiens
RTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 280 - DNA, 312 bp, Homo sapiens
  source 1..312: mol_type = other DNA
cccaaggccaaccccacggtcactctgttcccgccctcctctgaggagctccaagccaacaaggccacactagtgtgtctgatcagtgacttctacccgggagctgtgacagtggcttggaaggcagatggcagccccgtcaaggcgggagtggagacgaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 281 - protein, 104 aa, Homo sapiens
PKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 282 - DNA, 318 bp, Homo sapiens
  source 1..318: mol_type = other DNA
ggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca

SEQ ID NO: 283 - DNA, 318 bp, Homo sapiens; identical to SEQ ID NO: 282
  source 1..318: mol_type = other DNA

SEQ ID NO: 284 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 285 - DNA, 318 bp, Homo sapiens
  source 1..318: mol_type = other DNA
ggccagcctaaggccgctccttctgtgaccctgttccccccatcctccgaggaactgcaggctaacaaggccaccctcgtgtgcctgatcagcgacttctaccctggcgccgtgaccgtggcctggaaggctgatagctctcctgtgaaggccggcgtggaaaccaccaccccttccaagcagtccaacaacaaatacgccgcctcctcctacctgtccctgacccctgagcagtggaagtcccaccggtcctacagctgccaagtgacccacgagggctccaccgtggaaaagaccgtggctcctaccgagtgctcc

SEQ ID NO: 286 - DNA, 318 bp, Homo sapiens
  source 1..318: mol_type = other DNA
ggccagcctaaagctgcccccagcgtcaccctgtttcctccctccagcgaggagctccaggccaacaaggccaccctcgtgtgcctgatctccgacttctatcccggcgctgtgaccgtggcttggaaagccgactccagccctgtcaaagccggcgtggagaccaccacaccctccaagcagtccaacaacaagtacgccgcctccagctatctctccctgacccctgagcagtggaagtcccaccggtcctactcctgtcaggtgacccacgagggctccaccgtggaaaagaccgtcgcccccaccgagtgctcc

SEQ ID NO: 287 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 288 - DNA, 318 bp, Homo sapiens
  source 1..318: mol_type = other DNA
ggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 289 - protein, 106 aa, Homo sapiens; identical to SEQ ID NO: 287

SEQ ID NO: 290 - DNA, 312 bp, Homo sapiens
  source 1..312: mol_type = other DNA
cccaaggctgccccctcggtcactctgttcccaccctcctctgaggagcttcaagccaacaaggccacactggtgtgtctcataagtgacttctacccgggagccgtgacagttgcctggaaggcagatagcagccccgtcaaggcgggggtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagttgcccctacggaatgttca

SEQ ID NO: 291 - protein, 104 aa, Homo sapiens
PKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 292 - DNA, 318 bp, Homo sapiens
  source 1..318: mol_type = other DNA
ggtcagcccaaggctgccccctcggtcactctgttcccaccctcctctgaggagcttcaagccaacaaggccacactggtgtgtctcataagtgacttctacccggggccagtgacagttgcctggaaggcagatagcagccccgtcaaggcgggggtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacggaatgttca

SEQ ID NO: 293 - protein, 106 aa, Homo sapiens
GQPKAAPSVTLFPPSSEELQANKATLVCLISDFYPGPVTVAWKADSSPVKAGVETTTPSKQSNNKYAASSYLSLTPEQWKSHKSYSCQVTHEGSTVEKTVAPTECS

SEQ ID NO: 294 - DNA, 318 bp, Homo sapiens
  source 1..318: mol_type = other DNA
ggtcagcccaaggctgccccctcggtcactctgttcccaccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttca

SEQ ID NO: 295 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 296 - DNA, 318 bp, Homo sapiens
  source 1..318: mol_type = other DNA
ggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttca

SEQ ID NO: 297 - protein, 106 aa, Homo sapiens; identical to SEQ ID NO: 287

SEQ ID NO: 298 - DNA, 318 bp, Homo sapiens
  source 1..318: mol_type = other DNA
ggtcagcccaaggctgccccatcggtcactctgttcccgccctcctctgaggagcttcaagccaacaaggccacactggtgtgcctgatcagtgacttctacccgggagctgtgaaagtggcctggaaggcagatggcagccccgtcaacacgggagtggagaccaccacaccctccaaacagagcaacaacaagtacgcggccagcagctacctgagcctgacgcctgagcagtggaagtcccacagaagctacagctgccaggtcacgcatgaagggagcaccgtggagaagacagtggcccctgcagaatgttca

SEQ ID NO: 299 - protein, 106 aa, Homo sapiens
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 300 - DNA, 318 bp, Homo sapiens
  source 1..318: mol_type = other DNA
ggtcagcccaaggctgccccatcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 301 - protein, 106 aa, Homo sapiens
GQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 302 - DNA, 318 bp, Homo sapiens
  source 1..318: mol_type = other DNA
ggtcagcccaaggctgccccctcggtcactctgttcccaccctcctctgaggagcttcaagccaacaaggccacactggtgtgtctcgtaagtgacttcaacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctct

SEQ ID NO: 303 - protein, 106 aa, Homo sapiens
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS

SEQ ID NO: 304 - DNA, 978 bp, Homo sapiens
  source 1..978: mol_type = other DNA
gcttctaccaagggacccagcgtgttccctctggctccttgctccagatccacctccgagtctacagctgctctgggctgcctggtcaaggactactttcctgagcctgtgaccgtgtcttggaactctggcgctctgacatctggcgtgcacacattccctgctgtgctgcagtcctccggcctgtactctctgtcctctgtcgtgaccgtgccttcctctagcctgggcaccaagacctacacctgtaatgtggaccacaagccttccaacaccaaggtggacaagcgcgtggaatctaagtacggccctccttgtcctccatgtcctgctccagagtttgaaggcggcccttccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgcctagctccatcgaaaagaccatctccaaggccaagggccagcctcgagaaccccaggtttacaccctgcctccaagccaagaggaaatgaccaagaaccaggtgtccctgacctgcctcgtgaagggattctacccctccgatatcgccgtggaatgggagtctaatggccagccagagaacaactacaagacaacccctcctgtgctggactccgacggctccttctttctgtattcccgcctgaccgtggacaagtccagatggcaagagggcaacgtgttctcctgcagcgtgatgcacgaggccctgcacaatcactacacccagaagtccctgtctctgtccctgggc

SEQ ID NO: 305 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 306 - DNA, 990 bp, Homo sapiens
  source 1..990: mol_type = other DNA
gcttctaccaagggacccagcgttttccctctggctccatcctccaagtctacctctggcggaacagctgctctgggctgcctggtcaaggactactttcctgagccagtgaccgtgtcttggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagagtttgaaggcggcccttccgtgttcctgtttcctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgcctccatcgaaaagaccatctccaaggccaagggccagcctagggaaccccaggtttacaccttgccaccttctcgggacgagctgaccaagaaccaggtgtccctgacctgtctggttaagggcttctacccctccgatatcgccgtggaatgggagtctaatggccagcctgagaacaactacaagacaacccctcctgtgctggactccgacggctcattcttcctgtactccaagctgacagtggacaagtccagatggcagcagggcaacgtgttctcctgctctgtgatgcacgaggccctgcacaaccactacacccagaagtccctgtctctgagccccggcaag

SEQ ID NO: 307 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 308 - DNA, 353 bp, Homo sapiens
  source 1..353: mol_type = other DNA
atggagtttgggctgagctgggttttcctcgttgctcttttaagaggtgtccagtgtcaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaaga

SEQ ID NO: 309 - DNA, 359 bp, Homo sapiens
  source 1..359: mol_type = other DNA
atggagtttgggctgagctgggttttccttgttgctattttaaaaggtgtccagtgtgaggtgcagctggtggagtccgggggaggcttggtccagcctggggggtccctgaaactctcctgtgcagcctctgggttcaccttcagtggctctgctatgcactgggtccgccaggcttccgggaaagggctggagtgggttggccgtattagaagcaaagctaacagttacgcgacagcatatgctgcgtcggtgaaaggcaggttcaccatctccagagatgattcaaagaacacggcgtatctgcaaatgaacagcctgaaaaccgaggacacggccgtgtattactgtactagaca

SEQ ID NO: 310 - DNA, 356 bp, Homo sapiens
  source 1..356: mol_type = other DNA
atgaaacacctgtggttcttcctcctgctggtggcagctcccagatgggtcctgtcccaggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgtgcgagaga

SEQ ID NO: 311 - DNA, 353 bp, Homo sapiens
  source 1..353: mol_type = other DNA
atggagtttgggctgagctgggttttcctcgttgctcttttaagaggtgtccagtgtcaggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagaga

SEQ ID NO: 312 - DNA, 353 bp, Homo sapiens
  source 1..353: mol_type = other DNA
atggagtttgggctgagctggctttttcttgtggctattttaaaaggtgtccagtgtgaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaga

SEQ ID NO: 313 - DNA, 37 bp, Homo sapiens
  source 1..37: mol_type = other DNA
gtattatgattacgtttgggggagttatcgttatacc

SEQ ID NO: 314 - DNA, 31 bp, Homo sapiens
  source 1..31: mol_type = other DNA
gtattacgatattttgactggttattataac

SEQ ID NO: 315 - DNA, 31 bp, Homo sapiens
  source 1..31: mol_type = other DNA
gtattactatggttcggggagttattataac

SEQ ID NO: 316 - DNA, 17 bp, Homo sapiens
  source 1..17: mol_type = other DNA
ggtataactggaacgac

SEQ ID NO: 317 - DNA, 17 bp, Homo sapiens
  source 1..17: mol_type = other DNA
ggtacaactggaacgac

SEQ ID NO: 318 - DNA, 65 bp, Homo sapiens
  source 1..65: mol_type = other DNA
attactactactactacggtatggacgtctggggccaagggaccacggtcaccgtctcctcaggt

SEQ ID NO: 319 - DNA, 52 bp, Homo sapiens
  source 1..52: mol_type = other DNA
tgatgcttttgatatctggggccaagggacaatggtcaccgtctcttcaggt

SEQ ID NO: 320 - DNA, 351 bp, Homo sapiens
  source 1..351: mol_type = other DNA
atggacatgagggtccccgctcagctcctggggctcctgctgctctggttcccaggtgccaggtgtgacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttaccct

SEQ ID NO: 321 - DNA, 353 bp, Homo sapiens
  source 1..353: mol_type = other DNA
atgccctgggctctgctcctcctgaccctcctcactcactctgcagtgtcagtggtccaggcagggctgactcagccaccctcggtgtccaagggcttgagacagaccgccacactcacctgcactgggaacagcaacattgttggcaaccaaggagcagcttggctgcagcagcaccagggccaccctcccaaactcctatcctacaggaataacaaccggccctcagggatctcagagagattctctgcatccaggtcaggaaacacagcctccctgaccattactggactccagcctgaggacgaggctgactattactgctcagcattggacagcagcctcagtgctca

SEQ ID NO: 322 - DNA, 362 bp, Homo sapiens
  source 1..362: mol_type = other DNA
atgaggctccctgctcagctcctggggctgctaatgctctgggtctctggatccagtggggatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctcc

SEQ ID NO: 323 - DNA, 38 bp, Homo sapiens
  source 1..38: mol_type = other DNA
attcactttcggccctgggaccaaagtggatatcaaac

SEQ ID NO: 324 - DNA, 38 bp, Homo sapiens
  source 1..38: mol_type = other DNA
ttgggtgttcggcggagggaccaagctgaccgtcctag

SEQ ID NO: 325 - DNA, 38 bp, Homo sapiens
  source 1..38: mol_type = other DNA
gtggacgttcggccaagggaccaaggtggaaatcaaac

SEQ ID NO: 326 - DNA, 38 bp, Homo sapiens
  source 1..38: mol_type = other DNA
gctcactttcggcggagggaccaaggtggagatcaaac

SEQ ID NO: 327 - protein, 89 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNK

(19) United States
(12) Patent Application Publication
(10) Pub. No.: US 2024/0294651 A1
(43) Pub. Date: Sep. 5, 2024

(54) ANTIBODIES

(71) Applicant: KYMAB LIMITED, Cambridge (GB)

(72) Inventors: Margot BILLAUD, Cambridge (GB); Robert ROWLANDS, Cambridge (GB); John Matthew MCCOURT, Cambridge (GB); Matthew Stephen WAKE, Cambridge (GB); Morgane Marie LECOINTRE, Cambridge (GB); Matthew Joseph BARNES, Cambridge (GB); Catherine Jane HUTCHINGS, Cambridge (GB); Kirstie BENNETT, Cambridge (GB); Lisa STOTT, Cambridge (GB)

(73) Assignee: KYMAB LIMITED, Cambridge (GB)

(21) Appl. No.: 18/425,119

(22) Filed: Jan. 29, 2024

(30) Foreign Application Priority Data
Jan. 30, 2023 (EP) 23154055.0

(51) Int. Cl. C07K 16/28; A61K 39/00; A61K 45/06; A61P 35/00; C12N 15/63

(52) U.S. Cl. CPC C07K 16/2866; A61K 45/06; A61P 35/00; C12N 15/63; A61K 2039/505; C07K 2317/565; C07K 2317/71; C07K 2317/92

(57) ABSTRACT

The invention relates to antibodies or antigen-binding fragments thereof which specifically bind to CXCR4. The invention also relates to said antibodies for use in therapy, for use in treating cancer, and for use in treating a solid tumor. The invention also relates to pharmaceutical compositions comprising said antibodies, nucleic acids that encode said antibodies, vectors comprising said nucleic acids, and host cells comprising said nucleic acids or vectors.

### CROSS-REFERENCE TO RELATED APPLICATIONS

This application claims priority to European Patent Application No. 23154055.0, filed Jan. 30, 2023, the content of which is incorporated herein by reference in its entirety.

### SEQUENCE LISTING

The instant application contains a Sequence Listing which has been submitted electronically in XML format and is hereby incorporated by reference in its entirety. Said XML copy, created on Jan. 26, 2024, is named “122548.US018 20240129 Sequence Listing.xml” and is 394,964 bytes in size.

### FIELD OF THE INVENTION

Antibodies or antigen-binding fragments that specifically bind CXCR4 (C-X-C motif chemokine receptor 4) and their use in treating various diseases or conditions are described.

### INTRODUCTION

CXCR4 (also known as C-X-C motif chemokine receptor 4, CD184, FB22, Fusin, HM89, LCR1, leukocyte-derived seven transmembrane domain receptor (LESTR), Lipopolysaccharide-associated protein 3 (LAP-3) and stromal cell-derived factor 1 (SDF-1) receptor) (Uniprot ID: P61073) is a Class A, G-Protein-Coupled Receptor (GPCR) and part of the chemokine receptor family. CXCR4 shares the barrel-like structure common to GPCRs and comprises 7 transmembrane alpha-helices, 3 extracellular loops, 3 intracellular loops, an extracellular N-terminus and an intracellular C-terminus (Wu et al., (2010). Structures of the CXCR4 chemokine GPCR with small-molecule and cyclic peptide antagonists. Science. 330(6007):1066-1071, Zhu et al., (2013). Structure-based studies of chemokine receptors. Curr Opin Struct Biol. 23(4):539-546).

GPCRs are the largest family of membrane receptors in humans and numerous other species. In addition, GPCRs are considered the largest family of targets for approved drugs (Allen and Roth, (2011). Strategies to discover unexpected targets for drugs active at G protein-coupled receptors. Annu Rev Pharmacol Toxicol. 51:117-144; Rask-Andersen et al., (2014). The druggable genome: Evaluation of drug targets in clinical trials suggests major shifts in molecular class and indication. Annu Rev Pharmacol Toxicol. 54:9-26; Santos et al., (2017) and Oprea et al, (2018). Unexplored therapeutic opportunities in the human genome. Nat Rev Drug Discov. 17:317-332) A comprehensive map of molecular drug targets. Nat Rev Drug Discov. 16(1): 19-34). Numerous factors contribute to the wide utility of GPCR-targeted drugs, including their druggability, interaction with numerous types of chemokines and other ligands, and expression in the plasma membrane allowing extracellular targeting with molecules such as antibodies. This has resulted in greater than 35% of approved drugs targeting GPCRs and related upstream or downstream ligands or signalling pathways. Approximately ˜12% target the GPCRs directly (Hopkins and Groom, (2002). The druggable genome. Nat Rev Drug Discov. 1(9):727-730; Sriram and Insel, (2018). GPCRs as targets for approved drugs: How many targets and how many drugs?. Mol Pharmacol. 93(4):251-258). However, GPCRs are dynamic transmembrane proteins, making structural studies challenging. The CXCR4 crystal structure was solved in 2010 and was the first crystal structure of a peptide GPCR (Wu et al., (2010), Zhu et al., (2013) both supra).

The endogenous ligand for CXCR4 is CXCL12, which is also known as SDF-1 (Uniprot ID: P48061). CXCL12 is a highly conserved chemokine having 99% homology between human and mouse CXCL12 (Schabath et al., (1999). The murine chemokine receptor CXCR4 is tightly regulated during T cell development and activation. Journal of Leukocyte Biology. 66(6):996-1004). Many human CXCL12 isoforms exist, for example alpha, beta, gamma, delta, epsilon, theta and isoform 7. Additional ligands for CXCR4 have also been identified, which include Macrophage migration Inhibitory Factor (MIF) (Bernhagen et al., (2007). MIF is a noncognate ligand of CXC chemokine receptors in inflammatory and atherogenic cell recruitment. Nat Med. 13(5):587-596) and ubiquitin (Saini et al., (2010). CXC chemokine receptor 4 is a cell surface receptor for extracellular ubiquitin. J Biol Chem. 14; 285(20):15566-15576).

CXCL12 binding promotes a three-dimensional CXCR4 conformation favouring G_{αi} protein dissociation into α and βγ subunits. CXCL12 binding imparts different cellular activities such as inhibition of cAMP formation via inhibition of adenylyl cyclase activity, activation of phospholipase C(PLC)-β, generating diacylglycerol and inositol 1,4,5 trisphosphate (IP3), which controls the release of intracellular Ca²⁺. While inhibiting adenylyl cyclase, the receptor activates the NF-kβ, JAK-STAT, and PI3K-AKT pathways as well as mTOR, and the JNK/p38 MAPKs regulating cell survival, proliferation, and chemotaxis (Scala (2015). Molecular Pathways: Targeting the CXCR4-CXCL12 Axis—Untapped Potential in the Tumor Microenvironment. Clin Cancer Res. 21(19):4278-4285). Beta arrestin recruitment allows for receptor desensitisation through internalisation after CXCL12 binding (Teicher & Fricker (2010). CXCL12 (SDF-1)/CXCR4 pathway in cancer. Clin Cancer Res. 16(11):2927-2931; Cheng et al., (2000). β-Arrestin Differentially Regulates the Chemokine Receptor CXCR4-mediated Signalling and Receptor Internalization, and This Implicates Multiple Interaction Sites between β-Arrestin and CXCR4. J Biol Chem. 275(4): 2479-2485).

CXCR4 is expressed in many organs of the body and is highest on cells of the immune system and as a result is also found to be expressed highly in the bone marrow niche where these maturating immune cells originate (e.g. Myelopoietic cells, Erythropoietic cells, Lymphocytes, Monocytes and Blastic cells) (data from The Human Protein Atlas version 18.1 updated 15 Nov. 2018 https://www.proteinatlas.org/). CXCR4 is also found to be increased in tissues of endocrine origin (e.g. adrenal and thyroid tissues). CXCR4 expression has been identified on multiple cancer cells, for example breast, renal, pancreatic, ovarian, endometrial, head and neck, colorectal, stomach and lung (data from The Human Protein Atlas version 18.1 updated 15 Nov. 2018 https://www.proteinatlas.org/). CXCR4 is also involved in the later proliferation and metastasis of cancer cells (Guo et al., (2014). CXCL12-CXCR4 Axis Promotes Proliferation, Migration, Invasion, and Metastasis of Ovarian Cancer. Oncol Res. 22(5-6):247-258; Wei et al., (2018). Targeting CXC motif chemokine receptor 4 inhibits the proliferation, migration and angiogenesis of lung cancer cells. Oncol Lett. 16(3):3976-3982).

In addition to the role of CXCR4 in cancer, mutations in CXCR4 (mainly found in the C-terminus) have been associated with WHIM syndrome (warts, hypogammaglobulinemia, infections, and myelokathexis) (McDermott & Murphy (2019). WHIM syndrome: Immunopathogenesis, treatment and cure strategies. Immunol Rev. 287(1):91-102). Also, CXCR4 is a co-receptor for HIV-1 and has been investigated as a therapeutic target for HIV treatment (Chen (2019). Molecular Mechanism of HIV-1 Entry. Trends Microbiol. doi: 10.1016/j.tim.2019.06.002. [Epub ahead of print]).

### SUMMARY OF THE INVENTION

### Antibodies or Antigen-Binding Fragments

Disclosed herein are antibodies and antigen-binding fragments thereof that specifically bind to CXCR4, for example human CXCR4. In one embodiment, the antibody or antigen-binding fragment thereof specifically binds to surface expressed CXCR4. In another embodiment, the antibody or antigen-binding fragment thereof inhibits the binding of CXCL12 to CXCR4. In one embodiment, the antibody or antigen-binding fragment thereof inhibits the binding of CXCL12 to human CXCR4.

In one aspect, the invention provides an antibody or antigen-binding fragment thereof which specifically binds to CXCR4, wherein the antibody or antigen-binding fragment thereof comprises a VH domain, wherein the VH domain comprises: (i) a CDRH1 amino acid sequence of SEQ ID NOs: 10 or 13, or SEQ ID NOs: 10 or 13, each of which comprises 3, 2 or 1 amino acid substitution(s); (ii) a CDRH2 amino acid sequence of SEQ ID NOs: 11 or 14, or SEQ ID NOs: 11 or 14, each of which comprises 3, 2 or 1 amino acid substitution(s); (iii) a CDRH3 amino acid sequence of SEQ ID NOs: 12 or 15, or SEQ ID NOs: 12 or 15, each of which comprises 3, 2 or 1 amino acid substitution(s); and wherein the antibody or antigen-binding fragment thereof comprises a VL domain, wherein the VL domain comprises: (i) a CDRL1 amino acid sequence of SEQ ID NOs: 20 or 23, or SEQ ID NOs: 20 or 23, each of which comprises 3, 2 or 1 amino acid substitution(s); (ii) a CDRL2 amino acid sequence of SEQ ID NOs: 21 or 24, or SEQ ID NOs: 21 or 24, each of which comprises 3, 2 or 1 amino acid substitution(s); and (iii) a CDRL3 amino acid sequence of SEQ ID NOs: 22 or 25 or SEQ ID NOs: 22 or 25, each of which comprises 3, 2 or 1 amino acid substitution(s).

In one embodiment, the VH domain comprises: (i) a CDRH1 amino acid sequence of SEQ ID NOs: 10 or 13; (ii) a CDRH2 amino acid sequence of SEQ ID NOs: 11 or 14; (iii) a CDRH3 amino acid sequence of SEQ ID NOs: 12 or 15; and the VL domain comprises: (i) a CDRL1 amino acid sequence of SEQ ID NOs: 20 or 23; (ii) a CDRL2 amino acid sequence of SEQ ID NOs: 21 or 24; and (iii) CDRL3 amino acid sequence of SEQ ID NOs: 22 or 25, wherein each of CDRH1, CDRH2, CDRH3, CDRL1, CDRL2 and CDRL3 comprises up to one amino acid substitution, optionally a conservative amino acid substitution.

In one embodiment, the VH domain comprises: (i) a CDRH1 amino acid sequence of SEQ ID NOs: 10 or 13; (ii) a CDRH2 amino acid sequence of SEQ ID NOs: 11 or 14; (iii) a CDRH3 amino acid sequence of SEQ ID NOs: 12 or 15; and the VL domain comprises: (i) a CDRL1 amino acid sequence of SEQ ID NOs: 20 or 23; (ii) a CDRL2 amino acid sequence of SEQ ID NOs: 21 or 24; and (iii) CDRL3 amino acid sequence of SEQ ID NOs: 22 or 25, wherein up to one CDR, selected from CDRH1, CDRH2, CDRH3, CDRL1, CDRL2 and CDRL3, comprises up to one amino acid substitution, optionally a conservative amino acid substitution.

In one embodiment, the VH domain comprises: (i) a CDRH1 amino acid sequence of SEQ ID NOs: 10 or 13; (ii) a CDRH2 amino acid sequence of SEQ ID NOs: 11 or 14; (iii) a CDRH3 amino acid sequence of SEQ ID NOs: 12 or 15; and the VL domain comprises: (i) a CDRL1 amino acid sequence of SEQ ID NOs: 20 or 23; (ii) a CDRL2 amino acid sequence of SEQ ID NOs: 21 or 24; and (iii) CDRL3 amino acid sequence of SEQ ID NOs: 22 or 25.

In one embodiment, the VH domain comprises: (i) a CDRH1 amino acid sequence of SEQ ID NO: 13; (ii) a CDRH2 amino acid sequence of SEQ ID NO: 14; (iii) a CDRH3 amino acid sequence of SEQ ID NO: 15; and the VL domain comprises: (i) a CDRL1 amino acid sequence of SEQ ID NO: 23; (ii) a CDRL2 amino acid sequence of SEQ ID NO: 24; and (iii) CDRL3 amino acid sequence of SEQ ID NO: 25, wherein each of CDRH1, CDRH2, CDRH3, CDRL1, CDRL2 and CDRL3 comprises 0-3 amino acid substitutions, optionally a conservative amino acid substitution.

In one embodiment, the VH domain comprises: (i) a CDRH1 amino acid sequence of SEQ ID NO: 13; (ii) a CDRH2 amino acid sequence of SEQ ID NO: 14; (iii) a CDRH3 amino acid sequence of SEQ ID NO: 15; and the VL domain comprises: (i) a CDRL1 amino acid sequence of SEQ ID NO: 23; (ii) a CDRL2 amino acid sequence of SEQ ID NO: 24; and (iii) CDRL3 amino acid sequence of SEQ ID NO: 25, wherein each of CDRH1, CDRH2, CDRH3, CDRL1, CDRL2 and CDRL3 comprises up to one amino acid substitution, optionally a conservative amino acid substitution.

In one embodiment, the VH domain comprises: (i) a CDRH1 amino acid sequence of SEQ ID NO: 13; (ii) a CDRH2 amino acid sequence of SEQ ID NO: 14; (iii) a CDRH3 amino acid sequence of SEQ ID NO: 15; and the VL domain comprises: (i) a CDRL1 amino acid sequence of SEQ ID NO: 23; (ii) a CDRL2 amino acid sequence of SEQ ID NO: 24; and (iii) CDRL3 amino acid sequence of SEQ ID NO: 25, wherein up to one CDR, selected from CDRH1, CDRH2, CDRH3, CDRL1, CDRL2 and CDRL3, comprises up to one amino acid substitution, optionally a conservative amino acid substitution.

In one embodiment, the VH domain comprises: (i) a CDRH1 amino acid sequence of SEQ ID NO: 13; (ii) a CDRH2 amino acid sequence of SEQ ID NO: 14; (iii) a CDRH3 amino acid sequence of SEQ ID NO: 15; and the VL domain comprises: (i) a CDRL1 amino acid sequence of SEQ ID NO: 23; (ii) a CDRL2 amino acid sequence of SEQ ID NO: 24; and (iii) CDRL3 amino acid sequence of SEQ ID NO: 25.

In one embodiment, the VH domain comprises an amino acid sequence which is at least 90% identical to SEQ ID NO: 16; and/or the VL domain comprises an amino acid sequence which is at least 90% identical to SEQ ID NO: 26.

In a further embodiment, the VH domain comprises an amino acid sequence of SEQ ID NO: 16; and the VL domain comprises an amino acid sequence of SEQ ID NO: 26.

In one embodiment, the antibody or fragment is human, humanised or chimeric.

In one embodiment, the CXCR4 is human (optionally selected from SEQ ID NO: 1, SEQ ID NO: 2 and SEQ ID NO: 3). In one embodiment, the CXCR4 is rhesus and/or cynomolgus (optionally selected from SEQ ID NO: 7, SEQ ID NO: 8 and SEQ ID NO: 9).

In one embodiment, the antibody or fragment specifically binds to human, rhesus, cynomolgus and/or rodent CXCR4, optionally human and cynomolgus CXCR4, optionally wherein the binding is determined by surface plasmon resonance (SPR), flow cytometry, live cell imaging, ELISA or radioligand binding.

In one embodiment, the antibody or fragment comprises a constant region (CH and/or CL). In a further embodiment, the CH is (i) an IgG4 constant region, such as an IgG4-PE constant region (e.g. SEQ ID NO: 267 or SEQ ID NO: 305) or (ii) an IgG1 constant region, such as a IgG1 constant region comprising mutations that reduce binding to Fc-γ receptors and/or C1q as compared to wild-type (e.g. SEQ ID NO: 249 or 307). In a further embodiment, the CL is a kappa light chain constant region.

In one embodiment, the antibody or fragment comprises a heavy chain and a light chain, and the heavy chain amino acid sequence comprises an amino acid sequence that is at least 90% identical to SEQ ID NO: 18, and/or the light chain amino acid sequence comprises an amino acid sequence that is at least 90% identical to SEQ ID NO: 28. In a further embodiment, the heavy chain amino acid sequence comprises an amino acid sequence of SEQ ID NO: 18 and the light chain amino acid sequence comprises an amino acid sequence of SEQ ID NO: 28.

In one embodiment, the antibody or fragment inhibits the binding of CXCL12 to CXCR4.

In one embodiment, the antibody or fragment inhibits CXCL12-mediated inhibition of forskolin-stimulated cAMP with an IC50 of from 1 to 100 nM (e.g. 1 to 30 nM), optionally wherein inhibition of CXCR4-dependent CXCL12-mediated inhibition of cAMP is determined using a forskolin-stimulated cAMP signalling assay.

In one embodiment, the antibody or fragment inhibits beta-arrestin recruitment to CXCR4 with an IC50 of from 0.01 to 15 nM (e.g. 0.01 to 0.2 nM), optionally wherein beta-arrestin recruitment is determined using a functional cell-based reporter gene assay.

In one embodiment, the antibody or fragment inhibits the binding of CXCL12 to CXCR4 with an IC50 of from 0.2 to 4 nM (e.g. 0.2 to 1 nM), optionally wherein CXCL12 inhibition is determined using a HTRF assay.

In one embodiment, the antibody or fragment does not induce apoptosis in T-cells (optionally CD8+ T-cells), optionally wherein apoptosis is determined using flow cytometry.

In one embodiment, the antibody or fragment binds to cynomolgus CXCR4 with an EC50 of from 0.5 to 10 nM (e.g. 0.5 to 5 nM), optionally wherein cynomolgus CXCR4 binding is determined using flow cytometry.

In one embodiment, the antibody or fragment binds to human CXCR4 with a KD of from 0.2 to 2 nM, (e.g. 0.4 to 0.8 nM), optionally wherein binding affinity is determined using surface plasmon resonance (SPR).

In one embodiment, the antibody or fragment does not bind to CXCR7 (optionally wherein CXCR7 is human and is further optionally selected from SEQ ID NO: 4, SEQ ID NO: 5 or SEQ ID NO: 6), optionally wherein CXCR7 binding is determined using flow cytometry, radioligand binding, surface plasmon resonance (SPR), live cell imaging or ELISA.

In one embodiment, the antibody or fragment inhibits CXCL12 mediated response of T-cells with an IC50 of from 0.5 to 20 nM (e.g. 0.5 to 5 nM), wherein the inhibition is determined using label free dynamic mass redistribution assay in vitro.

In one embodiment, the antibody or fragment inhibits chemotaxis of CXCR4+ T-cells to CXCL12 with an IC50 of from 0.01 to 5 nM (e.g. 0.01 to 1 nM), wherein the inhibition is determined using live cell imaging or flow cytometry.

In one embodiment, the antibody or fragment increases mean CD45+ cell mobilisation compared to PBS, optionally wherein mobilisation is determined using flow cytometry.

In one embodiment, the antibody or fragment binds to CXCR4 homodimers.

In one embodiment, the antibody or fragment enables CD8+ T-cells to infiltrate a tumour.

One aspect of the invention provides the antibody or antigen-binding fragment as described herein for use in therapy.

A further aspect of the invention provides the antibody or fragment as described herein for use in treating cancer, optionally wherein the cancer is pancreatic cancer or pancreatic ductal cancer.

A further aspect of the invention provides the antibody or fragment as described herein for use in treating a solid tumor.

In one embodiment, treatment further comprises administering a further therapy, optionally wherein the further therapy comprises one or more further therapeutic agent(s) independently selected from the group consisting of an anti-PD-1 antibody or antigen-binding fragment thereof and an anti-PD-L1 antibody or antigen-binding fragment thereof; and/or optionally wherein the further therapy is selected from chemotherapy, radiotherapy and/or surgical removal of tumours.

In one embodiment, treatment further comprises administering a further therapeutic agent which is a PD-1/PD-L1 signalling inhibitor (e.g. a PD-1 antibody or antigen-binding fragment thereof, or a PD-L1 antibody or antigen-binding fragment thereof).

A further aspect of the invention provides a pharmaceutical composition comprising an antibody or fragment as described herein and a pharmaceutically acceptable excipient, diluent or carrier and optionally further comprising one or more further therapeutic agents.

A further aspect of the invention provides a nucleic acid that encodes (a) a VH domain and/or a VL domain of an antibody or fragment as described herein or (b) a heavy chain and/or a light chain of an antibody or fragment as described herein.

A further aspect of the invention provides a vector comprising the nucleic acid as described herein, optionally wherein the vector is a CHO or HEK293 vector.

A further aspect of the invention provides a host cell comprising the nucleic acid as described herein or the vector as described herein.

### DESCRIPTION OF THE FIGURES

FIGS. 1A-1H. Time dependent internalisation rates of anti-CXCR4 antibodies (FIG. 1A. CL-82458, FIG. 1B. CL-82558, FIG. 1C. CL-82574, FIG. 1D. CL-82658, FIG. 1E. CL-83083, FIG. 1F. Benchmark 1c, FIG. 1G. negative (IgG4-PE isotype) and FIG. 1H. relevant positive (human anti-CD20 antibody) controls. Figure shows internalisation rates at increasing concentrations of antibody. mAb conc; concentration of antibody used in assay, nanomolar concentration.

FIG. 2. Binding of anti-CXCR4 antibodies (in IgG4-PE format), benchmark antibodies (in IgG4-PE format) and IgG4-PE isotype control antibody to cynomolgus CXCR4. Representative data of anti-CXCR4 antibodies binding to cynomolgus CXCR4 expressed on CHO cells as assessed by flow cytometry. Data is representative of four independent experiments. Binding expressed as Geometric (Geo) mean.

FIG. 3. Non-binding of anti-CXCR4 (in IgG4-PE format), benchmark (in IgG4-PE format), IgG4-PE isotype control, Anti-CXCR7 mAb #11G8 and mouse IgG1 isotype control antibodies to CXCR7. Representative data of anti-CXCR4 antibodies not binding to CXCR7 expressed on CHO cells as assessed by flow cytometry. Data is representative of two independent experiments. Binding expressed as Geometric (Geo) mean.

FIG. 4. Mean inhibition percentages (±SD) obtained for anti-CXCR4 antibodies (in IgG4-PE format), benchmark antibodies (In IgG4-PE format) and IgG4-PE isotype control antibody incubated with Jurkat T-cells stimulated to migrate towards 30 nM of CXCL12. Data are representative of three independent experiments.

FIG. 5. Summary of anti-CXCR4 mediated inhibition of primary naïve T-cell migration. Plot represents the mean (±S.D.) of the percentage of maximum migration of naïve primary T-cells to 6.25 nM CXCL12 when pre-treated with different concentrations of anti-CXCR4 antibodies (in IgG4-PE format) and IgG4-PE isotype control in 3-5 independent donors.

FIG. 6. Inhibition of primary naïve T-cells by benchmark anti-CXCR4 antibodies. Plot represents the mean (±S.D.) of the percentage of maximum migration to 6.25 nM CXCL12 achieved for naïve T-cells pre-treated with different concentrations of benchmark anti-CXCR4 antibodies (in IgG4-PE format) and IgG4-PE isotype control for three independent donors.

FIG. 7. Percentage of CCRF-HSB-2 infiltration into IFNγ-treated HT-29 tumour spheroids following treatment with anti-CXCR4 antibodies (in IgG4-PE format) or IgG4-PE isotype control. Data are representative of five independent experiments (Mean f SD).

FIG. 8. Mobilisation of human CD45⁺ cells from NSG mice when treated with anti-CXCR4 antibodies (in IgG4-PE format). Total number of human CD45⁺ cells detected in the well of each blood sample, as recorded by the flow cytometer.

FIG. 9. Schematic of proposed mechanism of action of T-cell exclusion from tumour. Cancer associated fibroblasts (CAF), localised in the stroma secrete elevated levels of CXCL12 in response to local tumour-promoting conditions. CXCR4⁺ T-cells respond to the elevated CXCL12 gradients and are prevented from following other less established gradients (e.g. CXCL9, CXCL10 and/or CXCL11), which results in the T-cells remaining in the stroma. It is thought that the CXCL9, CXCL10 and/or CXCL11 gradients encourage T-cells to enter the tumour and effect killing of the malignant tumour cells.

FIG. 10. Inhibition of CXCR4 on T-cells by anti-CXCR4 antibodies prevents cells from responding to CXCL12 within the stroma. The T-cells are able to follow the other T-cell associated chemokine gradients (e.g. CXCL9, CXCL10 and/or CXCL11) which may allow for successful T-cell infiltration into tumour and subsequent tumour cell killing.

FIG. 11. Combination treatment using an anti-CXCR4 antibody and PD-1 inhibitor delays tumour growth compared to vehicle control.

### DETAILED DESCRIPTION

### 1. Definitions

Unless otherwise defined herein, scientific and technical terms shall have the meanings that are commonly understood by those of ordinary skill in the art. Further, unless otherwise required by context, singular terms shall include pluralities and plural terms shall include the singular.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempligratia, and is used herein to indicate a non-limiting example. Thus, the abbreviation “e.g.” is synonymous with the term “for example”.

In the specification and claims, the term “about” is used to modify, for example, the quantity of an ingredient in a composition, concentration, volume, process temperature, process time, yield, flow rate, pressure, and like values, and ranges thereof, employed in describing the embodiments of the disclosure. The term “about” refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods, and like proximate considerations. The term “about” also encompasses amounts that differ due to ageing of a formulation with a particular initial concentration or mixture, and amounts that differ due to mixing or processing a formulation with a particular initial concentration or mixture. Where modified by the term “about” the claims appended hereto include equivalents to these quantities.

As used herein, “administer” or “administration” refers to the act of injecting or otherwise physically delivering a substance as it exists outside the body (e.g., an anti-CXCR4 antibody or antigen-binding fragment provided herein) into a patient, such as by mucosal, intradermal, intravenous, intramuscular delivery and/or any other method of physical delivery described herein or known in the art. When a disease (or condition), or a symptom thereof, is being treated, administration of the substance typically occurs after the onset of the disease (or condition) or symptoms thereof. When a disease (or condition), or symptoms thereof, are being prevented, administration of the substance typically occurs before the onset of the disease (or condition) or symptoms thereof.

The term “affinity”, “binding affinity” or the like, as used herein refers to the degree or tendency to which two molecules combine with each other. In one embodiment, the affinity or binding affinity is specified as K_{D} or EC₅₀, a person skilled in the art would be able to calculate these values from data generated in an appropriate assay e.g. Surface Plasmon Resonance (SPR) (using Biacore™ or using the ProteOn XPR36™ (Bio-Rad™)), KinExA™ (Sapidyne Instruments, Inc), ForteBio Octet (Pall ForteBio Corp.), flow cytometry (e.g. Mirrorball™ fluorescence cytometer), live cell imaging, enzyme-linked immunosorbent assay (ELISA) or radioligand binding (preferably SPR, flow cytometry, live cell imagine, ELISA or radioligand binding); optionally used in combination with a programme such as GraphPad Prism. In one embodiment, the “affinity” or “binding affinity” is a measure of how strongly the antibody or antigen-binding fragment binds to the antigen, for example the target receptor (e.g. CXCR4). In one embodiment, the “affinity” or “binding affinity” is the strength with which an individual paratope (antigen-binding site) on an antibody or antigen-binding fragment binds to an epitope (of an antigen e.g. receptor), or is the strength with which the receptor (e.g. CXCR4) and ligand (e.g. CXCL12) interact. In a preferred embodiment, the affinity or binding affinity of the antibody or antigen-binding fragment for the receptor is in the nanomolar (nM) range or is in the picomolar (pM) range. The term “K_{D}”, as used herein, is intended to refer to the equilibrium dissociation constant of a particular antibody-antigen interaction. “Kₐ” is the association rate constant (also known as Kₒₙ) and “K_{d}” is the dissociation rate constant (also known as K_{off}).

The term “antagonist”, “antagonising” or the like, as used herein refers to the ability of an antibody or antigen-binding fragment thereof to bind to the target antigen and inhibit its interaction with one or more binding partners to a target antigen. In the case of CXCR4, an antagonist (e.g. antibody or antigen-binding fragment thereof) is able to antagonise the binding of a ligand (e.g. CXCL12) and prevent the biological effect of the ligand on the receptor (e.g. signalling of the ligand, conformational change of the receptor, activation of the receptor or interaction of the receptor with other cellular molecules). Known ligands of CXCR4 include CXCL12 (also known as SDF-1), Ubiquitin and MIF. Other CXCR4 ligands may not yet have been identified. Functional activity of CXCR4 may be measured in a cAMP assay, Beta-arrestin assay, CXCL12 inhibition assay, internalisation assay, apoptosis assay, DMR assay, chemotaxis assay, T-cell infiltration assay, migration assay or disease relevant models of cancer, which are well-known to those skilled in the art. Other assays are known to those skilled in the art. In a preferred embodiment, the antibody or antigen-binding fragment thereof is an antagonist, for example a full antagonist. In another embodiment, the antibody or antigen-binding fragment thereof is a partial antagonist. A partial antagonist partially inhibits (i.e. does not completely inhibit) the interaction of one or more binding partners to the target antigen.

The term “antibody”, “immunoglobulin” or “Ig” may be used interchangeably herein and means an immunoglobulin molecule that recognises and specifically binds to a target antigen, such as a protein, polypeptide, peptide, carbohydrate, polynucleotide, lipid, or combinations of the foregoing through at least one antigen recognition site within the variable region of the immunoglobulin molecule. The term “antibody” also refers to a Y-shaped glycoprotein with a molecular weight of approximately 150 kDa that is made up of four polypeptide chains: two light (L) chains and two heavy (H) chains. There are five types of mammalian Ig heavy chain constant region further described below. In mammals there are two types of immunoglobulin light chains, λ and κ. The “variable region” or “variable domain” of an antibody or antigen-binding fragment refers to the amino-terminal domains of the heavy or light chain of the antibody or antigen-binding fragment. The variable domains of the heavy chain and light chain may be referred to as “V_{H}” and “V_{L}”, respectively. These domains are generally the most variable parts of the antibody (relative to other antibodies of the same class) and contain the antigen-binding sites. The V_{H} and V_{L} are linked to heavy chain (C_{H}) and light chain (C_{L}) constant regions respectively.

The antibodies or antigen-binding fragments described herein may be oligoclonal, polyclonal, monoclonal (including full-length monoclonal antibodies), camelised, chimeric (e.g. mouse variable region and human constant region or human variable region and mouse constant region), CDR-grafted, multi-specific, bi-specific, catalytic, humanised, human, fully human, anti-idiotypic, including antibodies that can be labelled in soluble or bound form as well as fragments, variants, fusions or derivatives thereof, and any other modified immunoglobulin molecule comprising an antigen binding site so long as the antibodies or antigen-binding fragments exhibit the desired biological activity, either alone or in combination with other amino acid sequences provided by known techniques. In one embodiment, the antibody or antigen-binding fragment is human, fully human, humanised or chimeric. In a preferred embodiment, the antibody or antigen-binding fragment is a human antibody or antigen-binding fragment, optionally with non-human post-translational modifications (e.g. glycosylation). An antibody or antigen-binding fragment may be from any species. In one embodiment, the antibody or antigen-binding fragment is a mammalian or rodent antibody or antigen-binding fragment. Antibodies or antigen-binding fragments described herein can be naked or conjugated to other molecules such as toxins, radioisotopes, etc.

The term “antigen-binding domain,” “antigen-binding region,” “antigen-binding fragment,” and similar terms refer to that portion of an antibody which comprises the amino acid residues that interact with an antigen and confer on the binding agent its specificity and affinity for the antigen (e.g., the complementarity determining regions (CDRs)). The antigen-binding region can be derived from any animal species, such as rodents (e.g., mouse, rabbit, rat or hamster), chickens and humans. Preferably, the antigen-binding region will be of human origin. Throughout this specification, the word “fragment” in the context of an antibody is to be considered as an antigen-binding fragment of such an antibody.

The term “antigen-binding fragment” can include single-chain Fvs (scFv), single-chain antibodies, single domain antibodies, domain antibodies, Fv fragments, Fab fragments, F(ab′) fragments, F(ab′)₂ fragments, antibody fragments that exhibit the desired biological activity, disulphide-stabilised variable region (dsFv), dimeric variable region (diabody), anti-idiotypic (anti-Id) antibodies (including, e.g., anti-Id antibodies to antibodies), intrabodies, linear antibodies, single-chain antibody molecules and multispecific antibodies formed from antibody fragments and epitope-binding fragments of any of the above. In particular, antibodies and antibody fragments described herein can include immunoglobulin molecules and immunologically active fragments of immunoglobulin molecules, i.e., molecules that contain an antigen-binding site. Digestion of antibodies with the enzyme, papain, results in two identical antigen-binding fragments, known also as “Fab” fragments, and a “Fc” fragment, having no antigen-binding activity but having the ability to crystallize. “Fab” when used herein refers to a fragment of an antibody that includes one constant and one variable domain of each of the heavy and light chains. The term “Fc region” herein is used to define a C-terminal region of an immunoglobulin heavy chain, including native-sequence Fc regions and variant Fc regions. The “Fc fragment” refers to the carboxy-terminal portions of both H chains held together by disulphide bonds. The effector functions of antibodies are determined by sequences in the Fc region, the region which is also recognised by Fc receptors (FcR) found on certain types of cells. Digestion of antibodies with the enzyme, pepsin, results in a F(ab′)₂ fragment in which the two arms of the antibody molecule remain linked and comprise two-antigen-binding sites. The F(ab′)₂ fragment has the ability to crosslink antigen.

As used herein, “authorisation number” or “marketing authorisation number” refers to a number issued by a regulatory agency upon that agency determining that a particular medical product and/or composition may be marketed and/or offered for sale in the area under the agency's jurisdiction. As used herein “regulatory agency” refers to one of the agencies responsible for evaluating, e.g., the safety and efficacy of a medical product and/or composition and controlling the sales/marketing of such products and/or compositions in a given area. The Food and Drug Administration (FDA) in the US and the European Medicines Agency (EPA) in Europe are but two examples of such regulatory agencies. Other non-limiting examples can include SDA, MPA, MHPRA, IMA, ANMAT, Hong Kong Department of Health-Drug Office, CDSCO, Medsafe, and KFDA.

The term “bispecific antibody” means an antibody which comprises specificity for two target molecules. The term “bispecific antibody” includes formats such as biparatopic antibodies, DVD-Ig (see DiGiammarino et al., “Design and generation of DVD-Ig™ molecules for dual-specific targeting”, Meth. Mo. Biol., 2012, 889, 145-156), mAb² (see WO2008/003103), FIT-Ig (see WO2015/103072), mAb-dAb, dock and lock, Fab-arm exchange, SEEDbody, Triomab, LUZ-Y, Fcab, κλ-body, orthogonal Fab, scDiabody-Fc, diabody-Fc, tandem scFv-Fc, Fab-scFv-Fc, Fab-scFv, intrabody, BiTE, diabody, DART, TandAb, scDiabody, scDiabody-CH3, Diabody-CH3, Triple body, Miniantibody, minibody, TriBi minibody, scFv-CH3 KIH, scFv-CH-CL-scFv, F(ab′)2-scFv, scFv-KIH, Fab-scFv-Fc, tetravalent HCab, ImmTAC, knobs-in-holes, knobs-in-holes with common light chain, knobs-in-holes with common light chain and charge pairs, charge pairs, charge pairs with common light chain, DT-IgG, DutaMab, IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig and zybody. For a review of bispecific formats, see Spiess, C., et al., Mol. Immunol. (2015).

As used herein, the term “carrier” refers to a diluent, adjuvant (e.g., Freund's adjuvant), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Preferred carriers are Lipid A-based carriers and Gerbu adjuvants.

The term “CDR region” or “CDR” refers to the regions of an antibody or antigen-binding fragment variable domain which are hypervariable in sequence and/or form structurally defined loops. Generally, antigen-binding sites of an antibody include six CDR regions: three in the V_{H} (CDRH1, CDRH2, CDRH3), and three in the V_{L} (CDRL1, CDRL2, CDRL3). These regions of the heavy and light chains of an antibody confer antigen-binding specificity to the antibody. CDRs may be defined according to the Kabat system or IMGT system. Other systems may be used to define CDRs, which as the system devised by Chothia et al., (see Chothia, C. & Lesk, A. M., 1987, “Canonical structures for the hypervariable regions of immunoglobulins”, J. Mol. Biol., 196, 901-917). Other systems for determining CDRs include Martin (Enhanced Chothia) (Abhinandan and Martin (2008). Analysis and improvements to Kabat and structurally correct numbering of antibody variable domains. Mol Immunol. 45(14):3832-3839), AHo (Honegger and Plückthun (2001). Yet another numbering scheme for immunoglobulin variable domains: an automatic modelling and analysis tool. J Mol Biol. 309(3):657-670) and paratome (Kunik et al., (2012). Paratome: an online tool for systematic identification of antigen-binding regions in antibodies based on sequence or structure. Nucelic Acids Res. 40 (web server issue): W521-W524) numbering schemes. The term CDR or CDRs is used here to indicate one or several of these regions. A person skilled in the art is able to readily compare the different systems of nomenclature and determine whether a particular sequence may be defined as a CDR.

The term “chemotherapeutic agent” or “chemotherapy” refers to a therapeutic agent whose primary purpose is to destroy cancer cells, typically by interfering with the tumour cell's ability to grow or multiply. There are many different types of chemotherapeutic agents, with more than 50 approved chemotherapy drugs available. Chemotherapeutic drugs can be classified based on how they work. Alkylating drugs kill cancer cells by directly attacking DNA, the genetic material of the genes. Cyclophosphamide is an alkylating drug. Antimetabolites interfere with the production of DNA and keep cells from growing and multiplying. An example of an antimetabolite is 5-fluorouracil (5-FU). Anti-tumour antibiotics are made from natural substances such as fungi in the soil. They interfere with important cell functions, including production of DNA and cell proteins. Doxorubicin and bleomycin belong to this group of chemotherapy drugs. Plant alkaloids prevent cells from dividing normally. Vinblastine and vincristine are plant alkaloids obtained from the periwinkle plant. Steroid hormones slow the growth of some cancers that depend on hormones. For example, tamoxifen is used to treat breast cancers that depend on the hormone oestrogen for growth. DNA damage response (DDR) inhibitors, such as PARP inhibitors, block DNA repair mechanisms following single or double stranded breaks. In another embodiment, chemotherapeutic agents are agents that induce immunogenic cell death, for example platinum therapies, such as oxaliplatin. In one embodiment, the chemotherapy is a standard of care cytotoxic chemotherapy for the cancer being treated.

Examples of chemotherapeutic agents include Adriamycin, Doxorubicin, 5-Fluorouracil, Cytosine arabinoside (Ara-C), Cyclophosphamide, Thiotepa, Taxotere (docetaxel), Busulfan, Cytoxin, Gemcitabine, Paclitaxel, Doxorubicin, Taxol, Methotrexate, Cisplatin, Melphalan, Vinblastine, Bleomycin, Etoposide, Ifosfamide, Mitomycin C, Mitoxantrone, Vincreistine, Vinorelbine, Carboplatin, Teniposide, Daunomycin, Carminomycin, Aminopterin, Dactinomycin, Mitomycins, Esperamicins (see, U.S. Pat. No. 4,675,187), Melphalan, and other related nitrogen mustards. Suitable toxins and chemotherapeutic agents are described in Remington's Pharmaceutical Sciences, 19^{th} Ed. (Mack Publishing Co., 1995), and in Goodman and Gilman's The Pharmacological Basis of Therapeutics, 7^{th} Ed. (MacMillan Publishing Co., 1985). Other examples of chemotherapeutic agents include Irinotecan (liposomal), which is a topoisomerase I inhibitor. Other suitable toxins and/or chemotherapeutic agents are known to those of skill in the art. Leucovorin (folinic acid) is used in combination with chemotherapy drugs to either enhance effectiveness od said drug or function as a chemoprotectant, for example Leucovorin is used in combination with 5-Fluorouracil or methotrxate.

As used herein, the term “composition” is intended to encompass a product containing the specified ingredients (e.g., an antibody or antigen-binding fragment of the invention) in, optionally, the specified amounts, as well as any product which results, directly or indirectly, from combination of the specified ingredients in, optionally, the specified amounts.

As used herein the term “comprising” or “comprises” is used in reference to antibodies, antigen-binding fragments, uses, compositions, methods, and respective component(s) thereof, that are essential to the method or composition, yet open to the inclusion of unspecified elements, whether essential or not.

The term “consisting of” refers to antibodies, antigen-binding fragments, uses, compositions, methods, and respective components thereof as described herein, which are exclusive of any element not recited in that description of the embodiment.

The term “cross-reacts”, “cross-reactive” or like terms refers to specific binding between an antibody or antigen-binding fragment (e.g. anti-hCXCR4 antibody) and a similar target (e.g. CXCR4 of another species) or an unrelated target (e.g. CXCR7, CXCR3, CCR5 or other targets and/or ligands described elsewhere herein), which is not the target antigen (e.g. human CXCR4). In one embodiment, an antibody or antigen-binding fragment does not cross-react with an unrelated target if the binding is less than about 10% (e.g. 7%, 5%, 3% or 1%) of the binding of the antibody or antigen-binding to the desired target antigen (e.g. hCXCR4) as measured, e.g., by SPR. In another embodiment, an antibody or antigen-binding fragment cross-reacts with a similar target if the binding is greater than about 80% (e.g. 85%, 90%, 95%) of the binding, or is the same as the binding, of the antibody or antigen-binding to the desired target antigen (e.g. hCXCR4) as measured, e.g., by SPR.

In the context of a polypeptide, the term “derivative” as used herein refers to a polypeptide that comprises an amino acid sequence of a CXCR4 polypeptide, or an antibody or antigen-binding fragment that specifically binds to a CXCR4 polypeptide, which has been altered by the introduction of amino acid residue substitutions, deletions or additions. The term “derivative” as used herein also refers to a CXCR4 polypeptide or an antibody or antigen-binding fragment that specifically binds to a CXCR4 polypeptide, which has been chemically modified, e.g., by the covalent attachment of any type of molecule to the polypeptide. For example, but not by way of limitation, a CXCR4 polypeptide, a fragment of a CXCR4 polypeptide, or a CXCR4 antibody may be chemically modified, e.g., by glycosylation, acetylation, pegylation, phosphorylation, amidation, derivatisation by known protecting/blocking groups, proteolytic cleavage, linkage to a cellular ligand or other protein, etc. The derivatives are modified in a manner that is different from naturally occurring or starting peptide or polypeptides, either in the type or location of the molecules attached. Derivatives further include deletion of one or more chemical groups which are naturally present on the peptide or polypeptide. A derivative of a CXCR4 polypeptide or a CXCR4 antibody or antigen-binding fragment may be chemically modified by chemical modifications using techniques known to those of skill in the art, including, but not limited to specific chemical cleavage, acetylation, formulation, metabolic synthesis of tunicamycin, etc. Further, a derivative of a CXCR4 polypeptide or an anti-CXCR4 antibody or antigen-binding fragment may contain one or more non-classical amino acids. A polypeptide derivative possesses a similar or identical function as a CXCR4 polypeptide or an anti-CXCR4 antibody or antigen-binding fragment described herein.

An “effective amount” refers to an amount effective, at dosages and for periods of time necessary, to achieve the desired effect, including a therapeutic or prophylactic result. A “therapeutically effective amount” refers to the minimum concentration required to affect a measurable improvement or prevention of a particular disorder. A therapeutically effective amount herein may vary according to factors such as the disease state, age, sex, and weight of the patient, and the ability of the antibody or antigen-binding fragment to elicit a desired response in the individual. A therapeutically effective amount is also one in which toxic or detrimental effects of the antibody or antigen-binding fragment are outweighed by the therapeutically beneficial effects. A “prophylactically effective amount” refers to an amount effective, at the dosages and for periods of time necessary, to achieve the desired prophylactic result. In some embodiments, the effective amount of an antibody or antigen-binding fragment of the invention is from about 0.1 mg/kg (mg of antibody or antigen-binding fragment per kg weight of the subject) to about 100 mg/kg. In certain embodiments, an effective amount of an antibody or antigen-binding fragment is about 0.1 mg/kg, about 0.5 mg/kg, about 1 mg/kg, 3 mg/kg, 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg about 90 mg/kg or about 100 mg/kg (or a range therein). In some embodiments, “effective amount” as used herein also refers to the amount of an antibody or antigen-binding fragment of the invention to achieve a specified result (e.g., inhibition of a CXCR4 biological activity).

The term “epitope” as used herein refers to a localised region on the surface of an antigen, such as CXCR4 polypeptide, that is capable of being bound to one or more antigen-binding regions of an antibody or antigen-binding fragment, and that has antigenic or immunogenic activity in an animal, preferably a mammal, and most preferably in a human, that is capable of eliciting an immune response. An epitope having immunogenic activity is a portion of a polypeptide that elicits an antibody response in an animal. An epitope having antigenic activity is a portion of a polypeptide to which an antibody or antigen-binding fragment specifically binds as determined by any method well known in the art, for example, by immunoassays, some of which are described herein. Antigenic epitopes need not necessarily be immunogenic. Epitopes usually consist of chemically active surface groupings of molecules such as amino acids or sugar side chains and have specific three-dimensional structural characteristics as well as specific charge characteristics. A region of a polypeptide contributing to an epitope may be contiguous amino acids of the polypeptide or the epitope may come together from two or more non-contiguous regions of the polypeptide. The epitope may or may not be a three-dimensional surface feature of the antigen. In certain embodiments, a CXCR4 epitope is a three-dimensional surface feature of a CXCR4 polypeptide, which may be a monomer, a dimer (e.g. homodimer or heterodimer), a trimer, a tetramer or a multimer. In a preferred embodiment, a CXCR4 epitope is a three-dimensional surface feature of a monomeric or homodimeric CXCR4 polypeptide. In another embodiment, a CXCR4 epitope is a three-dimensional surface feature of a heterodimeric CXCR4 polypeptide, as further herein described. In other embodiments, a CXCR4 epitope is linear feature of a monomeric or homodimeric CXCR4 polypeptide.

The term “excipients” as used herein refers to inert substances which are commonly used as a diluent, vehicle, preservatives, binders, or stabilising agent for drugs and includes, but not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, non-ionic surfactant, etc.), saccharides (e.g., sucrose, maltose, trehalose, etc.) and polyols (e.g., mannitol, sorbitol, etc.). See, also, Remington's Pharmaceutical Sciences (1990) Mack Publishing Co., Easton, Pa., which is hereby incorporated by reference in its entirety.

The term “fusion protein” as used herein refers to a polypeptide that comprises an amino acid sequence of an antibody or antigen-binding fragment and an amino acid sequence of a heterologous polypeptide or protein (i.e., a polypeptide or protein not normally a part of the antibody or antigen-binding fragment (e.g., a non-anti-CXCR4 antibody or antigen-binding fragment)). The term “fusion” when used in relation to CXCR4 or to an anti-CXCR4 antibody refers to the joining of a peptide or polypeptide, or fragment, variant and/or derivative thereof, with a heterologous peptide or polypeptide. Preferably, the fusion protein retains the biological activity of the CXCR4 or anti-CXCR4 antibody. In certain embodiments, the fusion protein comprises a CXCR4 antibody V_{H} domain, V_{L} domain, V_{H} CDR (one, two or three V_{H} CDRs), and/or V_{L} CDR (one, two or three V_{L} CDRs), wherein the fusion protein specifically binds to a CXCR4 epitope.

The term “heavy chain” when used in reference to an antibody refers to five distinct types, called alpha (α), delta (δ), epsilon (ε), gamma (γ) and mu (μ), based on the amino acid sequence of the heavy chain constant domain. These distinct types of heavy chains are well known and give rise to five classes of antibodies, IgA, IgD, IgE, IgG and IgM, respectively, including four subclasses of IgG, namely IgG1, IgG2, IgG3 and IgG4, and two subclasses of IgA, namely IgA1 and IgA2. A preferred embodiment is an IgG isotype. In one embodiment, the heavy chain is a rodent heavy chain. In a preferred embodiment, the heavy chain is a human heavy chain. A heavy chain comprises a variable region (V_{H}) and a heavy chain constant region. Numbering of amino acid positions used for heavy chain constant regions in this specification is according to the EU Index (Kabat, E. A. (1991) Sequences of Proteins of Immunological Interest: Tabulation and Analysis of Amino Acid and Nucleic Acid Sequences of Precursors, V-Regions, C-Regions, J-Chain, T-Cell Receptors for Antigen T-Cell Surface Antigens, [Beta]2-Microglobulins, Major Histocompatibility Antigens, Thy-1, Complement, C-Reactive Protein, Thymopoietin, Integrins, PostGamma Globulin, [Alpha]2-Macroglobulins, and Other Related Proteins, 5th ed, National Institutes of Health).

The term “host cell” or like terms as used herein refers to the particular subject cell transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome. In one embodiment, the “host cell” is non-human. In one embodiment, the “host cell” is not a human totipotent cell.

A “human antibody” is an antibody that possesses an amino-acid sequence corresponding to that of an antibody produced by a human and/or has been made using any of the techniques for making human antibodies and specifically excludes a humanized antibody comprising non-human antigen-binding residues.

The term “humanized antibody” refers to a subset of chimeric antibodies in which a “CDR region” from a non-human immunoglobulin (the donor antibody) replaces residues from a CDR region in a human immunoglobulin (recipient antibody). In general, a humanized antibody will include substantially all of at least one, and typically two, variable domains, in which all or substantially all of the CDR regions correspond to those of a non-human immunoglobulin sequence, and all or substantially all of the framework regions are those of a human immunoglobulin sequence, although the framework regions may include one or more substitutions that improve antibody performance, such as binding affinity, isomerisation, immunogenicity, etc.

The term “IMGT numbering” and like terms are recognised in the art and refer to a system of numbering amino acid residues in order to compare the variable region sequences of immunoglobulins and T-cell receptors (Lefranc, M. P., 1997, “Unique database numbering system for immunogenetic analysis”, Immunol. Today, 18, 50). The IMGT numbering system relies on high conservation of the structure of the variable region. The IMGT numbering system takes into account and combines the definition of the framework (FR) and complementary determining regions (CDRs).

The term “inhibits”, “inhibition”, “inhibiting” and the like, as used herein refers to the ability of an antibody or antigen-binding fragment to bind to an epitope which either partially, substantially or completely prevents the binding of the ligand (e.g. CXCL12) to the receptor (e.g. CXCR4). If the epitope to which the antibody or antigen-binding fragment binds completely blocks the binding site of the ligand, then ligand binding is completely prevented (which may be a physical blocking—in the case of identical or overlapping epitopes—or steric blocking—where the antibody or antigen-binding fragment is large such that it prevents the ligand binding to its distinct epitope), and the ligand is not removed from circulation. The concentration of circulating ligand may therefore appear to be increased. If the epitope to which the antibody or antigen-binding fragment binds partially blocks the binding site of the ligand, the ligand may be able to bind, but only weakly (in the case of partial inhibition), or in a different orientation to the natural binding interaction. In this case, some of the ligand may be removed from circulation, but not as much as when the ligand binding site is completely free and available for binding. Inhibition thus refers to the physical interaction of ligand and receptor. IC₅₀ is the half maximal inhibitory concentration of an inhibitor (e.g. antibody or antigen-binding fragment disclosed herein). Inhibition can be measured by HTRF, which is described in more detail elsewhere herein and in Mathis (1995) Clinical Chemistry 41(9), 1391-1397. Inhibition can also be measured by time-resolved fluorescence energy transfer (TR-FRET) cell-based ligand inhibition assay (Zwier et al, (2010). A fluorescent ligand-binding alternative using Tag-lite Technology. J Biomol Screen. 15(10):1248-1259). Throughout this specification, the term “inhibit”, is interchangeable with the term “neutralise”. In any of the embodiments herein, by “inhibited” it is meant that the % specific binding at a given concentration or that the % maximum specific binding that is reached is comparable to (or the same as [within standard error]) that achieved when there is no ligand (e.g. CXCL12) provided for in the assay. Thus, in one embodiment, inhibition provides a reduction of the % specific binding (at either 50 nM, 30 nM, 10 nM, 1 nM or 0.1 nM [antibody or antigen-binding fragment]) of the ligand (e.g. CXCL12) in a range of from 50 to 100%, from 60 to 100%, from 70 to 100%, from 80% to 100%, for example from 85% to 100%, or from 90% to 100% as compared to isotype control. In another embodiment, the inhibition provides a reduction of the % specific binding of the ligand (e.g. CXCL12) (at either 50 nM, 30 nM, 10 nM, 1 nM or 0.1 nM [antibody or antigen-binding fragment]) in a range of from 95% to 100% as compared to isotype control. In another embodiment, the inhibition is a 100% reduction in % specific binding of the ligand (e.g. CXCL12) as compared to isotype control. In another embodiment, inhibition provides a reduction of the % maximum specific binding of the ligand (e.g. CXCL12) in a range of from 80% to 100%, for example from 85% to 100%, or from 90% to 100% as compared to isotype control. In another embodiment, the inhibition provides a reduction of the % maximum specific binding of the ligand (e.g. CXCL12) in a range of from 95% to 100% as compared to isotype control. In another embodiment, the full neutralisation is 100% reduction in % maximum specific binding of the ligand (e.g. CXCL12) as compared to isotype control. In one embodiment, an antibody or antigen-binding fragment which inhibits binding of CXCL12 to CXCR4 is an antagonist (e.g. full antagonist or partial antagonist etc) or an inverse agonist. An inverse agonist is an antibody or antigen-binding fragment which binds to the same target receptor as the agonist ligand (e.g. CXCR4) but induces a pharmacological response that is the opposite to the agonist induced response (e.g. if the agonist reduces cAMP levels, an inverse agonist may increase cAMP levels). Inverse agonism may be displayed when the receptor is constitutively active (it produces a pharmacological response in the absence of agonist). An inverse agonist can reduce the pharmacological response of the receptor in the absence of agonist, below the basal level of activity. In one embodiment, an anti-CXCR4 antibody (e.g. a CXCR4 inverse agonist) may reduce the pharmacological response of the receptor via one pathway and may not affect other pathways in which the receptor signals. In another embodiment, an anti-CXCR4 antibody (e.g. a CXCR4 inverse agonist) may increase cAMP levels in comparison to basal signalling of the receptor and also affect other CXCR4 signalling pathways.

As used herein, “injection device” refers to a device that is designed for carrying out injections, an injection including the steps of temporarily fluidically coupling the injection device to a person's tissue, typically the subcutaneous tissue. An injection further includes administering an amount of liquid drug into the tissue and decoupling or removing the injection device from the tissue. In some embodiments, an injection device can be an intravenous device or IV device, which is a type of injection device used when the target tissue is the blood within the circulatory system, e.g., the blood in a vein. A common, but non-limiting example of an injection device is a needle and syringe.

As used herein, “instructions” refers to a display of written, printed or graphic matter on the immediate container of an article, for example the written material displayed on a vial containing a pharmaceutically active agent, or details on the composition and use of a product of interest included in a kit containing a composition of interest. Instructions set forth the method of the treatment as contemplated to be administered or performed.

An “isolated” or “purified” antibody, antigen-binding fragment, protein or nucleic acid is one that has been identified, separated and/or recovered from a component of its production environment (e.g., natural or recombinant). For example, the antibody, antigen-binding fragment, protein or nucleic acid is substantially free of cellular material or other contaminating proteins from the cell or tissue source from which the antibody, antigen-binding fragment, protein or nucleic acid is derived, or substantially free of chemical precursors or other chemicals when chemically synthesised. The language “substantially free of cellular material” includes preparations of an antibody, antigen-binding fragment, protein or nucleic acid in which the desired product is separated from cellular components of the cells from which it is isolated or recombinantly produced. Thus, an antibody, antigen-binding fragment, protein or nucleic acid that is substantially free of cellular material includes preparations of desired material having less than about 30%, 20%, 10%, or 5% (by dry weight) of heterologous protein (also referred to herein as a “contaminating protein”). When the antibody, antigen-binding fragment, protein or nucleic acid is recombinantly produced, it is also preferably substantially free of culture medium, i.e., culture medium represents less than about 20%, 10%, or 5% of the volume of the protein preparation. When the antibody, antigen-binding fragment, protein or nucleic acid is produced by chemical synthesis, it is preferably substantially free of chemical precursors or other chemicals, i.e. it is separated from chemical precursors or other chemicals which are involved in the synthesis of the protein. Accordingly, such preparations have less than about 30%, 20%, 10%, 5% (by dry weight) of chemical precursors or compounds other than the desired material. In a preferred embodiment, antibodies or antigen-binding fragments of the invention are isolated and/or purified.

The term “isotype control” or like terms as used herein is an antibody or antigen-binding fragment generally of the same species (or mixture of species in the case of a chimera), and preferably having the same constant region as the test antibody or antigen-binding fragment, but wherein the V_{H} and V_{L} domains have either no specificity to the target antigen of interest (i.e. CXCR4), or have specificity for an antigen which is unrelated to the target antigen of interest (i.e. CXCR4) or are a non-binding combination of V_{H} and V_{L} to any other human target. When testing human variable-mouse constant chimeras, the isotype control may be a commercially available mouse IgG1 isotype control (e.g. available from Sigma Aldrich, catalogue number M9269). When testing a fully-human IgG4-PE formatted antibody, the isotope control is preferably an antibody having a human variable region and a human IgG4-PE constant region. A skilled person will be capable of identifying suitable antibodies as isotype controls.

The term “Kabat numbering,” and like terms are recognised in the art and refer to a system of numbering amino acid residues which are more variable (i.e. hypervariable) than other amino acid residues in the heavy chain variable regions of an antibody, or antigen-binding fragment thereof (Kabat et al., (1971) Ann. NY Acad. Sci. 190:382-391 and, Kabat et al., (1991) Sequences of Proteins of Immunological Interest, Fifth Edition, U.S. Department of Health and Human Services, NIH Publication No. 91-3242). For the heavy chain variable region, the hypervariable region typically ranges from amino acid positions 31 to 35 for CDR1, amino acid positions 50 to 65 for CDR2, and amino acid positions 95 to 102 for CDR3.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerization's, amidations) that may be present in minor amounts. Monoclonal antibodies are highly specific and are directed against a single antigenic determinant or epitope. In contrast, polyclonal antibody preparations typically include different antibodies directed against different antigenic determinants (or epitopes). The term “monoclonal antibody” as used herein encompasses both intact and full-length monoclonal antibodies (e.g. full length, four chain monoclonal antibodies) as well as antigen-binding fragments as elsewhere herein. Furthermore, “monoclonal antibody” refers to such antibodies made in any number of ways including, but not limited to, hybridoma, phage selection, recombinant expression, and transgenic animals. The monoclonal antibodies herein can include “chimeric” antibodies in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies that exhibit the desired biological activity.

The term “naturally occurring” or “native” when used in connection with biological materials such as nucleic acid molecules, polypeptides, host cells, and the like, refers to those which are found in nature and not manipulated by a human being (also known as wild-type). Native CXCR4 ligands include CXCL12 (SDF-1), MIF and Ubiquitin.

As used herein, “packaging” refers to how the components are organised and/or restrained into a unit fit for distribution and/or use. Packaging can include, e.g., boxes, bags, syringes, ampoules, vials, tubes, clamshell packaging, barriers and/or containers to maintain sterility, labelling, etc.

The terms “Percent (%) identity”, “Percent (%) amino acid sequence identity” (or “Percent (%) nucleotide sequence identity”), “Percent (%) identical to . . . ” “identity” and the like with respect to a peptide, polypeptide, antibody or antigen-binding fragment (or nucleotide) sequence are defined as the percentage of amino acid residues (or nucleotide bases) in a candidate sequence that are identical with the amino acid residues (or nucleotide bases) in the specific peptide or polypeptide sequence, after aligning the sequences and introducing gaps, if necessary, to achieve the best alignment, and not considering any conservative substitutions as part of the sequence identity. Alignment for the purposes of determining percent amino acid sequence identity can be achieved in various ways that are within the skill in the art, for instance, using publicly available computer software such as Needleman-Wunsch (NEEDLE program from Emboss), Basic Local Alignment Search Tool (BLAST™), BLAST-2, ALIGN or MEG ALIGN™ (DNASTAR) software. In one embodiment, % identity is the number of identical amino acid residues divided by the length of the alignment (X+Y), wherein the length of the alignment is the length of the longest sequence (e.g. X) plus the number of gapped residues in that sequence (Y). In a preferred embodiment, the alignment can be achieved using Needleman-Wunsch (NEEDLE program from Emboss), for example using a BLOSUM60 matrix, with a gap opening of 10 and gap extension of 0.5.

The term “pharmaceutically acceptable” as used herein means being approved by a regulatory agency of the Federal or a state government, or listed in the U.S. Pharmacopeia, European Pharmacopeia or other generally recognised Pharmacopeia for use in animals, and more particularly in humans.

As used herein, the term “polynucleotide”, “nucleotide”, “nucleic acid”, “nucleic acid molecule” and other similar terms are used interchangeable and include DNA, RNA, mRNA and the like.

As used herein, the terms “prevent,” “preventing,” “prevention” and “prophylaxis” refer to the total or partial inhibition of the development, recurrence, onset or spread of a CXCR4-mediated disease (or condition) and/or symptom related thereto, resulting from the administration of a therapy or combination of therapies provided herein (e.g., a combination of prophylactic or therapeutic agents, such as an antibody or antigen-binding fragment of the invention).

The term “radiotherapy” refers to a treatment that uses radiation (e.g. ionizing radiation) to kill cancer cells and/or reduce tumour mass. Radiotherapy can be used in combination with other therapeutics. Radiotherapy can be used in combination with chemotherapy. Radiotherapy may be used as a treatment after surgery to remove malignant tumours. Radiotherapy can be applied externally to the organism (e.g. human, dog, cat, mouse etc.), through implants (also known as brachytherapy) or by a radioisotope which can be administered by injections, capsules, drinks or implants. Radiotherapy is a term known in the art, and includes three-dimensional conformal radiation therapy (3D-CRT), image guided radiation therapy (IGRT), intensity modulated radiation therapy (IMRT), helical-tomotherapy, photon beam radiation therapy, proton beam radiation therapy, intraoperative radiation therapy (IORT), stereotactic radiosurgery and stereotactic body radiation therapy (SBRT). Radiotherapy can be applied externally with by a linear accelerator (LINAC) (also known as a linear partical accelerator).

The term “rodent” refers to mammals of the order Rodentia. In one embodiment, the rodent is a mammal with a single pair of continuously growing incisors in each of the upper and lower jaws. In one embodiment, the term “rodent” refers to one or more rodents selected from mouse, rat, squirrel, prairie dog, chipmunk, chinchilla, porcupine, beaver, capybara, gerbil, hamster and guinea pig. In one embodiment, rodent is the family Muridae. In one embodiment, rodent is murine. In a preferred embodiment, a rodent is mouse or rat.

The term “specifically binds” and the like herein refer to an antibody or antigen-binding fragment binding to the protein of interest (e.g. CXCR4) in a manner which is specific, rather than adhesion through polar interactions or other non-specific (but observable) binding. For example, an anti-CXCR4 antibody or antigen-binding fragment specifically binds to CXCR4 by virtue of its CDR regions interacting with their antigenic determinant (epitope) on CXCR4. An antibody or antigen-binding fragment thereof that specifically binds to a protein of interest (e.g. CXCR4 antigen) can be identified, for example, by immunoassays (e.g. Enzyme-linked immunosorbent assay (ELISA)), plate based multi-array (e.g. Meso Scale Discovery™ platform), SPR (e.g. Biacore™), flow cytometry (e.g. Mirrorball™ fluorescence cytometer), live cell imaging, radioimmunoassay's (RIA), radioligand binding or other techniques known to those of skill in the art. In a particular embodiment, binding is determined using flow cytometry, radioligand binding, SPR, live cell imaging or ELISA. Typically, a specific reaction will be at least twice background signal or noise and more typically more than 10 times (such as more than 15 times, more than 20 times, more than 50 times or more than 100 times) background. See, e.g., Paul, ed., 1989, Fundamental Immunology Second Edition, Raven Press, New York at pages 332-336 for a discussion regarding antibody specificity.

The term “subject” or “patient” refers to any animal, including, but not limited to, mammals, e.g. a non-human mammal. As used herein, the term “mammal” refers to any vertebrate animal that suckle their young and either give birth to living young (eutherian or placental mammals) or are egg-laying (Metatheria or nonplacental mammals). Examples of mammalian species include, but are not limited to, humans and other primates, including non-human primates such as chimpanzees and other apes and monkey species; farm animals such as cattle, sheep, pigs, goats and horses; domestic mammals such as dogs and cats; laboratory animals including rodents such as mice, rats (including cotton rats), hamsters and guinea pigs; birds, including domestic, wild and game birds such as chickens, turkeys and other gallinaceous birds, ducks, geese, and the like. In a particular embodiment, the subject is a human patient.

As used herein “substantially”, “substantially all” or “substantially tem” refers to at least about 60%, at least about 70%, at least about 75%, at least about 80%, at least about 85%, at least about 90%, at least about 95%, at least about 98%, at least about 99%, or about 100%.

As used herein, the term “therapeutic agent” refers to any agent that can be used in the treatment, management or amelioration of a CXCR4-mediated disease (or condition) and/or a symptom related thereto, which diseases (or conditions) are described elsewhere herein. In certain embodiments, the term “therapeutic agent” refers to an antibody or antigen-binding fragment of the invention. In certain other embodiments, the term “therapeutic agent” refers to an agent other than an antibody or antigen-binding fragment of the invention. Preferably, a therapeutic agent is an agent which is known to be useful for, or has been or is currently being used for the treatment, management or amelioration of a CXCR4-mediated disease (or condition) or one or more symptoms related thereto, for example a therapeutic agent that is considered by healthcare professionals to be a standard of care.

As used herein, the term “therapy” refers to any protocol, method and/or agent that can be used in the prevention, management, treatment and/or amelioration of a CXCR4-mediated disease or condition (e.g. cancer or any of the other diseases or conditions described herein). In certain embodiments, the terms “therapies” and “therapy” refer to a biological therapy, supportive therapy, and/or other therapies useful in the prevention, management, treatment and/or amelioration of a CXCR4-mediated disease or condition known to one of skill in the art such as medical personnel.

The terms “treat,” “treatment” and “treating” refer to the reduction or amelioration of the progression, severity, and/or duration of a CXCR4-mediated disease or condition (described elsewhere herein) resulting from the administration of one or more therapies (including, but not limited to, the administration of one or more therapeutic agents, such as an antibody or antigen-binding fragment of the invention or antigen-binding fragment thereof). In specific embodiments, such terms refer to the reduction or inhibition of the binding of CXCR4 ligands (e.g. CXCL12) to CXCR4, and/or the amelioration, reduction or inhibition of one or more symptoms associated with a CXCR4-mediated disease or condition (as described elsewhere herein, e.g. cancer).

The term “variable region” or “variable domain” refers to a portion of the light and heavy chains, typically the amino-terminal about 120 to 130 amino acids in the heavy chain and about 100 to 110 amino acids in the light chain, which differ extensively in sequence among antibodies and are used in the binding and specificity of each particular antibody for its particular antigen (e.g. CXCR4). The variability in sequence is concentrated in the CDR regions, while the more highly conserved regions in the variable domain are called framework regions (FR). In preferred embodiments, the variable region is a human variable region.

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al, (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al, (Eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al, eds.

Unless otherwise stated, the present invention was performed using standard procedures, as described, for example in Sambrook et al, Molecular Cloning: A Laboratory Manual (4 ed.), Cold Spring Harbor Laboratory Press, Cold Spring Harbor, N.Y., USA (2012); Molecular Biology 3^{rd} Edition, David Clark et al, Elsevier Science Publishing, Inc., New York, USA (2018); Molecular Biology of the Cell, 6^{th} Edition, Alberts et al, Garland Science, New York, USA (2014); Culture of Animal Cells: A Manual of Basic Technique and Specialized Applications, R. Ian Freshney, John Wiley & Sons, New Jersey USA (2011); Rang & Dale's Pharmacology 8^{th} Edition, Rang et al, Elsevier Churchill Livingstone., England (2016); Davis et al, Basic Methods in Molecular Biology, Elsevier Science Publishing, Inc., New York, USA (1995); Current Protocols in Protein Science (CPPS) (John E. Coligan, et al, ed., John Wiley and Sons, Inc.); Current Protocols in Cell Biology (CPCB) (Juan S. Bonifacino et al, ed., John Wiley and Sons, Inc.); Culture of Animal Cells: A Manual of Basic Technique by R. Ian Freshney, Publisher: Wiley-Liss; 5th edition (2005); and Animal Cell Culture Methods (Methods in Cell Biology, Vol. 57, Jennie P. Mather and David Barnes editors, Academic Press, 1st edition, 1998) which are all incorporated by reference herein in their entireties.

Other terms are defined herein within the description of the various embodiments of the invention.

### 2. CXCR4 Antibodies

The present invention provides antibodies or antigen-binding fragments thereof which specifically bind to CXCR4 (e.g. human CXCR4 [hCXCR4]). In a preferred embodiment, the antibody or antigen-binding fragment is a monoclonal antibody or antigen-binding fragment thereof (such as a fully human monoclonal antibody) that specifically binds CXCR4 (e.g., hCXCR4). In one embodiment, the antibody or antigen-binding fragment thereof is a full-length antibody. In a preferred embodiment, the antibody or antigen-binding fragment is a 4-chain antibody comprising 2 heavy chains and 2 light chains.

The present inventors have identified antibodies having specificity for CXCR4, which have a number of potential utilities and benefits over existing anti-CXCR4 antibodies. For example, the anti-CXCR4 antibodies or antigen-binding fragments described herein may have one or more of the following improved or beneficial properties:
a. Selectivity for inhibiting only one of the ligands of CXCR4 (e.g. inhibits CXCL12/CXCR4 interaction, but not CXCR4 interaction with other ligands (e.g. MIF or ubiquitin))
b. Specificity for CXCR4, inhibiting binding of multiple ligands (e.g. CXCL12, MIF and ubiquitin; CXCL12 and ubiquitin; or CXCL12 and MIF)
c. Induction of increased levels of infiltration of T-cells into a tumor (e.g. solid tumor) allowing reduction in tumor volume
d. Induction of low levels of apoptosis of T-cells
e. Improved or lower immunogenicity/lack of side effects
f. Improved solubility
g. Improved stability
h. Ease of formulation
i. Frequency of dosing and/or route of administration
j. Manufacturability (e.g. expression, ease of purification, isoforms, affinity for column during purification (e.g. Protein A or nickel), lack of dissociation of the heavy and light chains, dissociation of heavy chains, improved yield, reduced protein cleavage/clipping)
k. Improved efficacy and/or improved efficacy as part of a combination therapy

The anti-CXCR4 antibodies or antigen-binding fragments of the invention (including CL-82574, CL-82458, CL-82558, CL-82658, CL-83083, CL-83083-2, CL-82583, CL-82580, CL-82577, CL-82571, CL-82562, CL-82556, CL-82551, CL-82541, CL-82523, CL-82517, CL-82495, CL-82473, CL-82455, CL-83158, CL-148712 and CL-148729, which are described in detail below) are described with respect to the embodiments and configurations described herein. Unless otherwise stated, all embodiments and configurations, are to be read as being able to be combined with any other embodiment or configuration, unless such combination would not make technical sense or is explicitly stated otherwise. In particular, a reference to a specific embodiment includes a reference to any sub-embodiment (e.g. a reference to embodiment 3 in a subsequent embodiment specifically includes a reference back to embodiment 3a, embodiment 3b, etc), unless otherwise apparent from the context.

Throughout this specification, mention of CXCR4, in one embodiment is mammalian CXCR4. Examples of mammalian species include, but are not limited to, humans and other primates, including non-human primates such as chimpanzees and other apes and monkey species; farm animals such as cattle, sheep, pigs, goats and horses; domestic mammals such as dogs and cats; laboratory animals including rodents such as mice, rats (including cotton rats), hamsters, gerbils and guinea pigs. In one embodiment, the CXCR4 is rodent CXCR4 (e.g. mouse or rat CXCR4). In another embodiment, CXCR4 is rhesus and/or cynomolgus CXCR4 (optionally selected from SEQ ID NO: 7, SEQ ID NO: 8 or SEQ ID NO: 9). In a preferred embodiment, the CXCR4 is human CXCR4 (optionally wherein human CXCR4 is selected from SEQ ID NO: 1, SEQ ID NO: 2 or SEQ ID NO: 3). In another embodiment, the CXCR4 is cell surface expressed CXCR4.

CXCR4 is a GPCR and like most GPCRs, multiple isoforms exist. In one embodiment, the antibodies or antigen-binding fragments thereof are capable of interacting with all isoforms of hCXCR4. The amino acid sequence for human CXCR4 (hCXCR4) can be found in Uniprot ID: P61073 (SEQ ID NO:2). To date, two hCXCR4 isoforms are identified in the Uniprot database: isoform 1 is 352 amino acids in length, whereas isoform 2 is 356 amino acids in length (Duquenne et al., (2014). The Two Human CXCR4 Isoforms Display Different HIV Receptor Activities: Consequences for the Emergence of X4 Strains. J Immunol. 193(8):4188-4194). The amino acids 1-5 MEGIS (in the N-terminus of the receptor) in isoform 1 are replaced with MSIPLPLLQ in isoform 2. In a preferred embodiment, the antibody or antigen-binding fragment thereof specifically binds to either isoform 1 (SEQ ID NO: 2) or isoform 2. In a preferred embodiment, the antibody or antigen-binding fragment thereof specifically binds to both isoform 1 and 2. It will be appreciated that not all isoforms may have been identified yet.

In another embodiment, the CXCR4 protein has undergone post-translational modifications (for example glycosylation, phosphorylation, ubiquitination or sulphation). In one embodiment, the CXCR4 has undergone glycosylation (N-linked and/or O-linked) on amino acids N11, S18 and/or N176 (reference sequence: isoform 1).

Throughout the embodiments herein, CXCR4 may be wild-type human CXCR4. Additionally, or alternatively, the human CXCR4 may be a variant CXCR4, for example a SNP, a polymorphic variant, a truncated variant, a mutated variant or a variant having frameshift mutations. In one embodiment, the variant CXCR4 is encoded by an amino acid sequence comprising one or more single nucleotide polymorphisms (SNPs) (e.g. selected from the group consisting of CXCR4-I261I, CXCR4-K68K and CXCR4-F93S (Petersen et al., (2005). Risk for HIV-1 Infection Associated with a Common CXCL12 (SDF1) Polymorphism and CXCR4 Variation in an African Population, J. Acquir. Immune Defic. Syndr. 40(5): 521-526)). Single point mutations to the intracellular C-terminal region of CXCR4 can result in variants being produced which are common in Warts, Hypogammaglobulinemia, Infections, and Myelokathexis (WHIM) or associated diseases (e.g. Waldenström macroglobulinaemia). Additionally, or alternatively, the variant CXCR4 may be encoded by an amino acid sequence comprising one or more mutations, for example nonsense mutations (resulting in truncations of the amino acid sequence, e.g. E343K, E343X, S338X, G336X and R334X (wherein X terminates the sequence) (Liu et al., (2012) WHIM syndrome caused by a single amino acid substitution in the carboxy-tail of chemokine receptor CXCR4. Blood. 120(1):181-189). A frameshift mutation is caused by the insertion or deletion of one or more nucleotides, which results in the translation of the genetic code in an unnatural reading frame from the position of the mutation onwards (e.g. to the end of the reading frame or to a terminator nucleic acid sequence). In another embodiment, the variant CXCR4 is encoded by an amino acid sequence comprising a frameshift mutation (e.g. S341 frameshift, S339 frameshift, H315 frameshift, T318 frameshift, R322 frameshift, L326 frameshift, K327 frameshift and 1328 frameshift (Liu et al., (2012) supra, and Poulain et al., (2016). Genomic Landscape of CXCR4 Mutations in Waldenström Macroglobulinemia. Clin. Cancer Res. 22(6):1480-1488)). In another embodiment, the variant CXCR4 is encoded by an amino acid sequence comprising a deletion between amino acids 334-352, 338-352 or 343-352 (reference sequence: isoform 1) (Hernandez et al., (2003). Mutations in the chemokine receptor gene CXCR4 are associated with WHIM syndrome, a combined immunodeficiency disease. Nat Genet. 34:70-74; Treon et al., (2014). Somatic mutations in MYD88 and CXCR4 are determinants of clinical presentation and overall survival in Waldenstrom macroglobulinemia. Blood. 123:2791-2796.). Some patients may express any of these variant CXCR4 proteins and therefore antibodies or antigen-binding fragments which are able to inhibit these variant CXCR4 may be useful in treating or preventing a different or broader range of patients (for example a broader range of patient if the antibodies or antigen-binding fragments are cross reactive between wild type CXCR4 and the variant CXCR4). Thus, in one embodiment, certain CXCR4-mediated diseases or conditions may be treated or prevented when the anti-CXCR4 antibody or antigen-binding fragment binds to a variant CXCR4 having a mutation independently selected from a deletion between amino acids 334-352, 338-352 or 343-352 (reference sequence: isoform 1); or a variant CXCR4 encoded by an amino acid sequence comprising one or more SNPs selected from the group consisting of CXCR4-I261I, CXCR4-K68K and CXCR4-F93S; or a variant CXCR4 encoded by an amino acid sequence comprising one or more of nonsense mutations selected from the group consisting of E343K, E343X, S338X, G336X or R334X; or a variant CXCR4 encoded by an amino acid sequence comprising one or more frameshift mutations selected from the group consisting of S341 frameshift, S339 frameshift, H315 frameshift, T318 frameshift, R322 frameshift, L326 frameshift, K327 frameshift and 1328 frameshift.

In an example the binding site(s) of the antibody or antigen-binding fragment are selected from a plurality (e.g., library) of binding sites. For example, the plurality of binding sites comprises or consists of a plurality of 4-chain antibodies or fragments thereof, e.g., Fabs or scFvs. Suitable methods for producing pluralities of binding sites for screening include phage display (producing a phage display library of antibody binding sites), ribosome display (producing a ribosome display library of antibody binding sites), yeast display (producing a yeast display library of antibody binding sites), mammalian display, covalent display (e.g. cis-display) or immunisation of a non-human vertebrate (e.g. a rodent, e.g., a mouse or rat, e.g., a Velocimouse™, Intelliselec™ transgenic mouse, Xenomouse™, Aliva Mouse™, HuMab Mouse™, Omnimouse™, Omnirat™ MeMo Mouse™) OmniFlic™, OmniChicken™, and OmniClic™) with hCXCR4 protein or a hCXCR4 epitope, followed by isolation of a repertoire of antibody-producing cells (e.g. a B-cell, plasma cell or plasmablast repertoire) and/or a repertoire of isolated antibodies or antigen-binding fragments.

The inventors have provided the following anti-CXCR4 antibodies, each of which specifically bind to CXCR4:

CL-82574 has a heavy chain variable region (V_{H}) amino acid sequence of SEQ ID NO: 16, comprising the CDRH1 amino acid sequence of SEQ ID NO: 10 (IMGT) or SEQ ID NO: 13 (Kabat), the CDRH2 amino acid sequence of SEQ ID NO: 11 (IMGT) or SEQ ID NO: 14 (Kabat), and the CDRH3 amino acid sequence of SEQ ID NO: 12 (IMGT) or SEQ ID NO: 15 (Kabat). The heavy chain nucleic acid sequence of the V_{H} domain is SEQ ID NO: 17. CL-82574 has a light chain variable region (V_{L}) amino acid sequence of SEQ ID NO: 26, comprising the CDRL1 amino acid sequence of SEQ ID NO: 20 (IMGT) or SEQ ID NO: 23 (Kabat), the CDRL2 amino acid sequence of SEQ ID NO: 21 (IMGT) or SEQ ID NO: 24 (Kabat), and the CDRL3 amino acid sequence of SEQ ID NO: 22 (IMGT) or SEQ ID NO: 25 (Kabat). The light chain nucleic acid sequence of the V_{L} domain is SEQ ID NO: 27. The V_{H} domain may be combined with any of the heavy chain constant region sequences described herein, e.g. SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 267, SEQ ID NO: 269, SEQ ID NO: 305 or SEQ ID NO: 307. The V_{L} domain may be combined with any of the light chain constant region sequences described herein, e.g. SEQ ID NO: 271, SEQ ID NO: 273, SEQ ID NO: 275, SEQ ID NO: 277, SEQ ID NO: 279, SEQ ID NO: 281, SEQ ID NO: 284, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301 or SEQ ID NO: 303. A full-length heavy chain amino acid sequence is SEQ ID NO: 18 (heavy chain nucleic acid sequence SEQ ID NO: 19). A full-length light chain amino acid sequence is SEQ ID NO: 28 (light chain nucleic acid sequence SEQ ID NO: 29). The heavy chain variable region is made by recombination of human IGHV3-33*01 (SEQ ID NO: 311), IGHD1-20*01 (SEQ ID NO: 316) and IGHJ3*02 (SEQ ID NO: 319) gene segments. The light chain variable region is made by recombination of human IGKV1-17*01 (SEQ ID NO: 320) and IGKJ3*01 (SEQ ID NO: 323) gene segments.

CL-82458 has a heavy chain variable region (V_{H}) amino acid sequence of SEQ ID NO:36, comprising the CDRH1 amino acid sequence of SEQ ID NO: 30 (IMGT) or SEQ ID NO: 33 (Kabat), the CDRH2 amino acid sequence of SEQ ID NO: 31 (IMGT) or SEQ ID NO: 34 (Kabat), and the CDRH3 amino acid sequence of SEQ ID NO: 32 (IMGT) or SEQ ID NO: 35 (Kabat). The heavy chain nucleic acid sequence of the V_{H} domain is SEQ ID NO: 37. CL-82458 has a light chain variable region (V_{L}) amino acid sequence of SEQ ID NO: 46, comprising the CDRL1 amino acid sequence of SEQ ID NO: 40 (IMGT) or SEQ ID NO: 43 (Kabat), the CDRL2 amino acid sequence of SEQ ID NO: 41 (IMGT) or SEQ ID NO: 44 (Kabat), and the CDRL3 amino acid sequence of SEQ ID NO: 42 (IMGT) or SEQ ID NO: 45 (Kabat). The light chain nucleic acid sequence of the V_{L} domain is SEQ ID NO: 47. The V_{H} domain may be combined with any of the heavy chain constant region sequences described herein, e.g. SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 267, SEQ ID NO: 269, SEQ ID NO: 305 or SEQ ID NO: 307. The V_{L} domain may be combined with any of the light chain constant region sequences described herein, e.g. SEQ ID NO: 271, SEQ ID NO: 273, SEQ ID NO: 275, SEQ ID NO: 277, SEQ ID NO: 279, SEQ ID NO: 281, SEQ ID NO: 284, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301 or SEQ ID NO: 303. A full-length heavy chain amino acid sequence is SEQ ID NO: 38 (heavy chain nucleic acid sequence SEQ ID NO: 39). A full-length light chain amino acid sequence is SEQ ID NO: 48 (light chain nucleic acid sequence SEQ ID NO: 49). The heavy chain variable region is made by recombination of human IGHV3-23*04 (SEQ ID NO: 312), IGHD1-1*01 (SEQ ID NO: 317) and IGHJ6*02 (SEQ ID NO: 318) gene segments. The light chain variable region is made by recombination of human IGKV1-17*01 (SEQ ID NO: 320) and IGKJ4*01 (SEQ ID NO: 326) gene segments.

CL-82558 has a heavy chain variable region (V_{H}) amino acid sequence of SEQ ID NO: 56, comprising the CDRH1 amino acid sequence of SEQ ID NO: 50 (IMGT) or SEQ ID NO: 53 (Kabat), the CDRH2 amino acid sequence of SEQ ID NO: 51 (IMGT) or SEQ ID NO: 54 (Kabat), and the CDRH3 amino acid sequence of SEQ ID NO: 52 (IMGT) or SEQ ID NO: 55 (Kabat). The heavy chain nucleic acid sequence of the V_{H} domain is SEQ ID NO: 57. CL-82558 has a light chain variable region (V_{L}) amino acid sequence of SEQ ID NO: 66, comprising the CDRL1 amino acid sequence of SEQ ID NO: 60 (IMGT) or SEQ ID NO: 63 (Kabat), the CDRL2 amino acid sequence of SEQ ID NO: 61 (IMGT) or SEQ ID NO: 64 (Kabat), and the CDRL3 amino acid sequence of SEQ ID NO: 62 (IMGT) or SEQ ID NO: 65 (Kabat). The light chain nucleic acid sequence of the V_{L} domain is SEQ ID NO: 67. The V_{H} domain may be combined with any of the heavy chain constant region sequences described herein, e.g. SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 267, SEQ ID NO: 269, SEQ ID NO: 305 or SEQ ID NO: 307. The V_{L} domain may be combined with any of the light chain constant region sequences described herein, e.g. SEQ ID NO: 271, SEQ ID NO: 273, SEQ ID NO: 275, SEQ ID NO: 277, SEQ ID NO: 279, SEQ ID NO: 281, SEQ ID NO: 284, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301 or SEQ ID NO: 303. A full-length heavy chain amino acid sequence is SEQ ID NO: 58 (heavy chain nucleic acid sequence SEQ ID NO: 59). A full-length light chain amino acid sequence is SEQ ID NO: 68 (light chain nucleic acid sequence SEQ ID NO: 69). The heavy chain variable region is made by recombination of human IGHV4-31*03 (SEQ ID NO: 310), IGHD3-10*01 (SEQ ID NO: 315) and IGHJ6*02 (SEQ ID NO: 318) gene segments. The light chain variable region is made by recombination of human IGKV2-28*01 (SEQ ID NO: 322) and IGKJ1*01 (SEQ ID NO: 325) gene segments.

CL-82658 has a heavy chain variable region (V_{H}) amino acid sequence of SEQ ID NO: 76, comprising the CDRH1 amino acid sequence of SEQ ID NO: 70 (IMGT) or SEQ ID NO: 73 (Kabat), the CDRH2 amino acid sequence of SEQ ID NO: 71 (IMGT) or SEQ ID NO: 74 (Kabat), and the CDRH3 amino acid sequence of SEQ ID NO: 72 (IMGT) or SEQ ID NO: 75 (Kabat). The heavy chain nucleic acid sequence of the V_{H} domain is SEQ ID NO: 77. CL-82658 has a light chain variable region (V_{L}) amino acid sequence of SEQ ID NO: 86, comprising the CDRL1 amino acid sequence of SEQ ID NO: 80 (IMGT) or SEQ ID NO: 83 (Kabat), the CDRL2 amino acid sequence of SEQ ID NO: 81 (IMGT) or SEQ ID NO: 84 (Kabat), and the CDRL3 amino acid sequence of SEQ ID NO: 82 (IMGT) or SEQ ID NO: 85 (Kabat). The light chain nucleic acid sequence of the V_{L} domain is SEQ ID NO: 87. The V_{H} domain may be combined with any of the heavy chain constant region sequences described herein, e.g. SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 267, SEQ ID NO: 269, SEQ ID NO: 305 or SEQ ID NO: 307. The V_{L} domain may be combined with any of the light chain constant region sequences described herein, e.g. SEQ ID NO: 271, SEQ ID NO: 273, SEQ ID NO: 275, SEQ ID NO: 277, SEQ ID NO: 279, SEQ ID NO: 281, SEQ ID NO: 284, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301 or SEQ ID NO: 303. A full-length heavy chain amino acid sequence is SEQ ID NO: 78 (heavy chain nucleic acid sequence SEQ ID NO: 79). A full-length light chain amino acid sequence is SEQ ID NO: 88 (light chain nucleic acid sequence SEQ ID NO: 89). The heavy chain variable region is made by recombination of human IGHV3-30*18 (SEQ ID NO: 308), IGHD3-16*02 (SEQ ID NO: 313) and IGHJ6*02 (SEQ ID NO: 318) gene segments. The light chain variable region is made by recombination of human IGKV1-17*01 (SEQ ID NO: 320) and IGKJ3*01 (SEQ ID NO: 323) gene segments.

CL-83083 has a heavy chain variable region (V_{H}) amino acid sequence of SEQ ID NO: 96, comprising the CDRH1 amino acid sequence of SEQ ID NO: 90 (IMGT) or SEQ ID NO: 93 (Kabat), the CDRH2 amino acid sequence of SEQ ID NO: 91 (IMGT) or SEQ ID NO: 94 (Kabat), and the CDRH3 amino acid sequence of SEQ ID NO: 92 (IMGT) or SEQ ID NO: 95 (Kabat). The heavy chain nucleic acid sequence of the V_{H} domain is SEQ ID NO: 97. CL-83083 has a light chain variable region (V_{L}) amino acid sequence of SEQ ID NO: 106, comprising the CDRL1 amino acid sequence of SEQ ID NO: 100 (IMGT) or SEQ ID NO: 103 (Kabat), the CDRL2 amino acid sequence of SEQ ID NO: 101 (IMGT) or SEQ ID NO: 104 (Kabat), and the CDRL3 amino acid sequence of SEQ ID NO: 102 (IMGT) or SEQ ID NO: 105 (Kabat). The light chain nucleic acid sequence of the V_{L} domain is SEQ ID NO: 107. The V_{H} domain may be combined with any of the heavy chain constant region sequences described herein, e.g. SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 267, SEQ ID NO: 269, SEQ ID NO: 305 or SEQ ID NO: 307. The V_{L} domain may be combined with any of the light chain constant region sequences described herein, e.g. SEQ ID NO: 271, SEQ ID NO: 273, SEQ ID NO: 275, SEQ ID NO: 277, SEQ ID NO: 279, SEQ ID NO: 281, SEQ ID NO: 284, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301 or SEQ ID NO: 303. A full-length heavy chain amino acid sequence is SEQ ID NO: 98 (heavy chain nucleic acid sequence SEQ ID NO: 99). A full-length light chain amino acid sequence is SEQ ID NO: 108 (light chain nucleic acid sequence SEQ ID NO: 109). The heavy chain variable region is made by recombination of human IGHV3-73*02 (SEQ ID NO: 309), IGHD3-9*01 (SEQ ID NO: 314) and IGHJ6*02 (SEQ ID NO: 318) gene segments. The light chain variable region is made by recombination of human IGLV10-54*02 (SEQ ID NO: 321) and IGLJ3*02 (SEQ ID NO: 324) gene segments.

CL-83083-2 has a heavy chain variable region (V_{H}) amino acid sequence of SEQ ID NO: 96, comprising the CDRH1 amino acid sequence of SEQ ID NO: 90 (IMGT) or SEQ ID NO: 93 (Kabat), the CDRH2 amino acid sequence of SEQ ID NO: 91 (IMGT) or SEQ ID NO: 94 (Kabat), and the CDRH3 amino acid sequence of SEQ ID NO: 92 (IMGT) or SEQ ID NO: 95 (Kabat). The heavy chain nucleic acid sequence of the V_{H} domain is SEQ ID NO: 97. CL-83083-2 has a light chain variable region (V_{L}) amino acid sequence of SEQ ID NO: 106, comprising the CDRL1 amino acid sequence of SEQ ID NO: 100 (IMGT) or SEQ ID NO: 103 (Kabat), the CDRL2 amino acid sequence of SEQ ID NO: 101 (IMGT) or SEQ ID NO: 104 (Kabat), and the CDRL3 amino acid sequence of SEQ ID NO: 102 (IMGT) or SEQ ID NO: 105 (Kabat). The light chain nucleic acid sequence of the V_{L} domain is SEQ ID NO: 107. The V_{H} domain may be combined with any of the heavy chain constant region sequences described herein, e.g. SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 267, SEQ ID NO: 269, SEQ ID NO: 305 or SEQ ID NO: 307. The V_{L} domain may be combined with any of the light chain constant region sequences described herein, e.g. SEQ ID NO: 271, SEQ ID NO: 273, SEQ ID NO: 275, SEQ ID NO: 277, SEQ ID NO: 279, SEQ ID NO: 281, SEQ ID NO: 284, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301 or SEQ ID NO: 303. A full-length heavy chain amino acid sequence is SEQ ID NO: 98 (heavy chain nucleic acid sequence SEQ ID NO: 99). A full-length light chain amino acid sequence is SEQ ID NO: 110 (light chain nucleic acid sequence SEQ ID NO: 111). The heavy chain variable region is made by recombination of human IGHV3-73*02 (SEQ ID NO: 309), IGHD3-9*01 (SEQ ID NO: 314) and IGHJ6*02 (SEQ ID NO: 318) gene segments. The light chain variable region is made by recombination of human IGLV10-54*02 (SEQ ID NO: 321) and IGLJ3*02 (SEQ ID NO: 324) gene segments.

CL-82583 has a heavy chain variable region (V_{H}) amino acid sequence of SEQ ID NO: 112, comprising a CDRH1, CDRH2 and CDRH3 amino acid sequences as defined by IMGT or Kabat from V_{H} amino acid sequence SEQ ID NO: 112. The heavy chain nucleic acid sequence of the V_{H} domain is SEQ ID NO: 113. CL-82583 has a light chain variable region (V_{L}) amino acid sequence of SEQ ID NO: 116, comprising the CDRL1, CDRL2 and CDRL3 amino acid sequences as defined by IMGT or Kabat from V_{L} amino acid sequence of SEQ ID NO: 116. The light chain nucleic acid sequence of the V_{L} domain is SEQ ID NO: 117. The V_{H} domain may be combined with any of the heavy chain constant region sequences described herein, e.g. SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 267, SEQ ID NO: 269, SEQ ID NO: 305 or SEQ ID NO: 307. The V_{L} domain may be combined with any of the light chain constant region sequences described herein, e.g. SEQ ID NO: 271, SEQ ID NO: 273, SEQ ID NO: 275, SEQ ID NO: 277, SEQ ID NO: 279, SEQ ID NO: 281, SEQ ID NO: 284, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301 or SEQ ID NO: 303. A full-length heavy chain amino acid sequence is SEQ ID NO: 114 (heavy chain nucleic acid sequence SEQ ID NO: 115). A full-length light chain amino acid sequence is SEQ ID NO: 118 (light chain nucleic acid sequence SEQ ID NO: 119). The heavy chain variable region is made by recombination of human IGHV3-33*01 (SEQ ID NO: 311), IGHD1-1*01 (SEQ ID NO: 317) and IGHJ3*02 (SEQ ID NO: 319) gene segments. The light chain variable region is made by recombination of human IGKV1-17*01 (SEQ ID NO: 320) and IGKJ3*01 (SEQ ID NO: 323) gene segments.

CL-82580 has a heavy chain variable region (V_{H}) amino acid sequence of SEQ ID NO: 120, comprising a CDRH1, CDRH2 and CDRH3 amino acid sequences as defined by IMGT or Kabat from V_{H} amino acid sequence SEQ ID NO: 120. The heavy chain nucleic acid sequence of the V_{H} domain is SEQ ID NO: 121. CL-82580 has a light chain variable region (V_{L}) amino acid sequence of SEQ ID NO: 124, comprising the CDRL1, CDRL2 and CDRL3 amino acid sequences as defined by IMGT or Kabat from V_{L} amino acid sequence of SEQ ID NO: 124. The light chain nucleic acid sequence of the V_{L} domain is SEQ ID NO: 125. The V_{H} domain may be combined with any of the heavy chain constant region sequences described herein, e.g. SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 267, SEQ ID NO: 269, SEQ ID NO: 305 or SEQ ID NO: 307. The V_{L} domain may be combined with any of the light chain constant region sequences described herein, e.g. SEQ ID NO: 271, SEQ ID NO: 273, SEQ ID NO: 275, SEQ ID NO: 277, SEQ ID NO: 279, SEQ ID NO: 281, SEQ ID NO: 284, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301 or SEQ ID NO: 303. A full-length heavy chain amino acid sequence is SEQ ID NO: 122 (heavy chain nucleic acid sequence SEQ ID NO: 123). A full-length light chain amino acid sequence is SEQ ID NO: 126 (light chain nucleic acid sequence SEQ ID NO:127). The heavy chain variable region is made by recombination of human IGHV1-8*01, IGHD3-10*01 (SEQ ID NO: 315) and IGHJ6*02 (SEQ ID NO: 318) gene segments. The light chain variable region is made by recombination of human IGKV1-5*03 and IGKJ1*01 (SEQ ID NO: 325) gene segments.

CL-82577 has a heavy chain variable region (V_{H}) amino acid sequence of SEQ ID NO: 128, comprising a CDRH1, CDRH2 and CDRH3 amino acid sequences as defined by IMGT or Kabat from V_{H} amino acid sequence SEQ ID NO: 128. The heavy chain nucleic acid sequence of the V_{H} domain is SEQ ID NO: 129. CL-82577 has a light chain variable region (V_{L}) amino acid sequence of SEQ ID NO: 132, comprising the CDRL1, CDRL2 and CDRL3 amino acid sequences as defined by IMGT or Kabat from V_{L} amino acid sequence of SEQ ID NO: 132. The light chain nucleic acid sequence of the V_{L} domain is SEQ ID NO: 133. The V_{H} domain may be combined with any of the heavy chain constant region sequences described herein, e.g. SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 267, SEQ ID NO: 269, SEQ ID NO: 305 or SEQ ID NO: 307. The V_{L} domain may be combined with any of the light chain constant region sequences described herein, e.g. SEQ ID NO: 271, SEQ ID NO: 273, SEQ ID NO: 275, SEQ ID NO: 277, SEQ ID NO: 279, SEQ ID NO: 281, SEQ ID NO: 284, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301 or SEQ ID NO: 303. A full-length heavy chain amino acid sequence is SEQ ID NO: 130 (heavy chain nucleic acid sequence SEQ ID NO: 131). A full-length light chain amino acid sequence is SEQ ID NO: 134 (light chain nucleic acid sequence SEQ ID NO: 135). The heavy chain variable region is made by recombination of human IGHV4-31*03 (SEQ ID NO: 310), IGHD3-10*01 (SEQ ID NO: 315) and IGHJ6*02 (SEQ ID NO: 318) gene segments. The light chain variable region is made by recombination of human IGKV2-28*01 (SEQ ID NO: 322) and IGKJ1*01 (SEQ ID NO: 325) gene segments.

CL-82571 has a heavy chain variable region (V_{H}) amino acid sequence of SEQ ID NO: 136, comprising a CDRH1, CDRH2 and CDRH3 amino acid sequences as defined by IMGT or Kabat from V_{H} amino acid sequence SEQ ID NO: 136. The heavy chain nucleic acid sequence of the V_{H} domain is SEQ ID NO: 137. CL-82571 has a light chain variable region (V_{L}) amino acid sequence of SEQ ID NO: 140, comprising the CDRL1, CDRL2 and CDRL3 amino acid sequences as defined by IMGT or Kabat from V_{L} amino acid sequence of SEQ ID NO: 140. The light chain nucleic acid sequence of the V_{L} domain is SEQ ID NO: 141. The V_{H} domain may be combined with any of the heavy chain constant region sequences described herein, e.g. SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 267, SEQ ID NO: 269, SEQ ID NO: 305 or SEQ ID NO: 307. The V_{L} domain may be combined with any of the light chain constant region sequences described herein, e.g. SEQ ID NO: 271, SEQ ID NO: 273, SEQ ID NO: 275, SEQ ID NO: 277, SEQ ID NO: 279, SEQ ID NO: 281, SEQ ID NO: 284, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301 or SEQ ID NO: 303. A full-length heavy chain amino acid sequence is SEQ ID NO: 138 (heavy chain nucleic acid sequence SEQ ID NO: 139). A full-length light chain amino acid sequence is SEQ ID NO: 142 (light chain nucleic acid sequence SEQ ID NO: 143). The heavy chain variable region is made by recombination of human IGHV3-21*03, IGHD5-18*01 and IGHJ6*02 (SEQ ID NO: 318) gene segments. The light chain variable region is made by recombination of human IGKV1D-13*d01 and IGKJ4*01 (SEQ ID NO: 326) gene segments.

CL-82562 has a heavy chain variable region (V_{H}) amino acid sequence of SEQ ID NO: 144, comprising a CDRH1, CDRH2 and CDRH3 amino acid sequences as defined by IMGT or Kabat from V_{H} amino acid sequence SEQ ID NO: 144. The heavy chain nucleic acid sequence of the V_{H} domain is SEQ ID NO: 145. CL-82562 has a light chain variable region (V_{L}) amino acid sequence of SEQ ID NO: 148, comprising the CDRL1, CDRL2 and CDRL3 amino acid sequences as defined by IMGT or Kabat from V_{L} amino acid sequence of SEQ ID NO: 148. The light chain nucleic acid sequence of the V_{L} domain is SEQ ID NO: 149. The V_{H} domain may be combined with any of the heavy chain constant region sequences described herein, e.g. SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 267, SEQ ID NO: 269, SEQ ID NO: 305 or SEQ ID NO: 307. The V_{L} domain may be combined with any of the light chain constant region sequences described herein, e.g. SEQ ID NO: 271, SEQ ID NO: 273, SEQ ID NO: 275, SEQ ID NO: 277, SEQ ID NO: 279, SEQ ID NO: 281, SEQ ID NO: 284, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301 or SEQ ID NO: 303. A full-length heavy chain amino acid sequence is SEQ ID NO: 146 (heavy chain nucleic acid sequence SEQ ID NO: 147). A full-length light chain amino acid sequence is SEQ ID NO: 150 (light chain nucleic acid sequence SEQ ID NO: 151). The heavy chain variable region is made by recombination of human IGHV3-30*18 (SEQ ID NO: 308), IGHD1-20*01 (SEQ ID NO: 316) and IGHJ6*02 (SEQ ID NO: 318) gene segments. The light chain variable region is made by recombination of human IGKV1-17*01 (SEQ ID NO: 320) and IGKJ1*01 (SEQ ID NO: 325) gene segments.

CL-82556 has a heavy chain variable region (V_{H}) amino acid sequence of SEQ ID NO: 152, comprising a CDRH1, CDRH2 and CDRH3 amino acid sequences as defined by IMGT or Kabat from V_{H} amino acid sequence SEQ ID NO: 152. The heavy chain nucleic acid sequence of the V_{H} domain is SEQ ID NO: 153. CL-82556 has a light chain variable region (V_{L}) amino acid sequence of SEQ ID NO: 156, comprising the CDRL1, CDRL2 and CDRL3 amino acid sequences as defined by IMGT or Kabat from V_{L} amino acid sequence of SEQ ID NO: 156. The light chain nucleic acid sequence of the V_{L} domain is SEQ ID NO: 157. The V_{H} domain may be combined with any of the heavy chain constant region sequences described herein, e.g. SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 267, SEQ ID NO: 269, SEQ ID NO: 305 or SEQ ID NO: 307. The V_{L} domain may be combined with any of the light chain constant region sequences described herein, e.g. SEQ ID NO: 271, SEQ ID NO: 273, SEQ ID NO: 275, SEQ ID NO: 277, SEQ ID NO: 279, SEQ ID NO: 281, SEQ ID NO: 284, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301 or SEQ ID NO: 303. A full-length heavy chain amino acid sequence is SEQ ID NO: 154 (heavy chain nucleic acid sequence SEQ ID NO: 155). A full-length light chain amino acid sequence is SEQ ID NO: 158 (light chain nucleic acid sequence SEQ ID NO: 159). The heavy chain variable region is made by recombination of human IGHV3-33*01 (SEQ ID NO: 311), IGHD1-20*01 (SEQ ID NO: 316) and IGHJ3*02 (SEQ ID NO: 319) gene segments. The light chain variable region is made by recombination of human IGKV1-17*01 (SEQ ID NO: 320) and IGKJ3*01 (SEQ ID NO: 323) gene segments.

CL-82551 has a heavy chain variable region (V_{H}) amino acid sequence of SEQ ID NO: 160, comprising a CDRH1, CDRH2 and CDRH3 amino acid sequences as defined by IMGT or Kabat from V_{H} amino acid sequence SEQ ID NO: 160. The heavy chain nucleic acid sequence of the V_{H} domain is SEQ ID NO: 161. CL-82551 has a light chain variable region (V_{L}) amino acid sequence of SEQ ID NO: 164, comprising the CDRL1, CDRL2 and CDRL3 amino acid sequences as defined by IMGT or Kabat from V_{L} amino acid sequence of SEQ ID NO: 164. The light chain nucleic acid sequence of the V_{L} domain is SEQ ID NO: 165. The V_{H} domain may be combined with any of the heavy chain constant region sequences described herein, e.g. SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 267, SEQ ID NO: 269, SEQ ID NO: 305 or SEQ ID NO: 307. The V_{L} domain may be combined with any of the light chain constant region sequences described herein, e.g. SEQ ID NO: 271, SEQ ID NO: 273, SEQ ID NO: 275, SEQ ID NO: 277, SEQ ID NO: 279, SEQ ID NO: 281, SEQ ID NO: 284, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301 or SEQ ID NO: 303. A full-length heavy chain amino acid sequence is SEQ ID NO: 162 (heavy chain nucleic acid sequence SEQ ID NO: 163). A full-length light chain amino acid sequence is SEQ ID NO: 166 (light chain nucleic acid sequence SEQ ID NO: 167). The heavy chain variable region is made by recombination of human IGHV1-8*01, IGHD3-10*01 (SEQ ID NO: 315) and IGHJ6*02 (SEQ ID NO: 318) gene segments. The light chain variable region is made by recombination of human IGKV1-5*03 and IGKJ1*01 (SEQ ID NO: 325) gene segments.

CL-82541 has a heavy chain variable region (V_{H}) amino acid sequence of SEQ ID NO: 168, comprising a CDRH1, CDRH2 and CDRH3 amino acid sequences as defined by IMGT or Kabat from V_{H} amino acid sequence SEQ ID NO: 168. The heavy chain nucleic acid sequence of the V_{H} domain is SEQ ID NO: 169. CL-82541 has a light chain variable region (V_{L}) amino acid sequence of SEQ ID NO: 172, comprising the CDRL1, CDRL2 and CDRL3 amino acid sequences as defined by IMGT or Kabat from V_{L} amino acid sequence of SEQ ID NO: 172. The light chain nucleic acid sequence of the V_{L} domain is SEQ ID NO: 173. The V_{H} domain may be combined with any of the heavy chain constant region sequences described herein, e.g. SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 267, SEQ ID NO: 269, SEQ ID NO: 305 or SEQ ID NO: 307. The V_{L} domain may be combined with any of the light chain constant region sequences described herein, e.g. SEQ ID NO: 271, SEQ ID NO: 273, SEQ ID NO: 275, SEQ ID NO: 277, SEQ ID NO: 279, SEQ ID NO: 281, SEQ ID NO: 284, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301 or SEQ ID NO: 303. A full-length heavy chain amino acid sequence is SEQ ID NO: 170 (heavy chain nucleic acid sequence SEQ ID NO: 171). A full-length light chain amino acid sequence is SEQ ID NO: 174 (light chain nucleic acid sequence SEQ ID NO: 175). The heavy chain variable region is made by recombination of human IGHV4-31*03 (SEQ ID NO: 310), IGHD3-10*01 (SEQ ID NO: 315) and IGHJ6*02 (SEQ ID NO: 318) gene segments. The light chain variable region is made by recombination of human IGKV2-28*01 (SEQ ID NO: 322) and IGKJ1*01 (SEQ ID NO: 325) gene segments.

CL-82523 has a heavy chain variable region (V_{H}) amino acid sequence of SEQ ID NO: 176, comprising a CDRH1, CDRH2 and CDRH3 amino acid sequences as defined by IMGT or Kabat from V_{H} amino acid sequence SEQ ID NO: 176. The heavy chain nucleic acid sequence of the V_{H} domain is SEQ ID NO: 177. CL-82523 has a light chain variable region (V_{L}) amino acid sequence of SEQ ID NO: 180, comprising the CDRL1, CDRL2 and CDRL3 amino acid sequences as defined by IMGT or Kabat from V_{L} amino acid sequence of SEQ ID NO: 180. The light chain nucleic acid sequence of the V_{L} domain is SEQ ID NO: 181. The V_{H} domain may be combined with any of the heavy chain constant region sequences described herein, e.g. SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 267, SEQ ID NO: 269, SEQ ID NO: 305 or SEQ ID NO: 307. The V_{L} domain may be combined with any of the light chain constant region sequences described herein, e.g. SEQ ID NO: 271, SEQ ID NO: 273, SEQ ID NO: 275, SEQ ID NO: 277, SEQ ID NO: 279, SEQ ID NO: 281, SEQ ID NO: 284, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301 or SEQ ID NO: 303. A full-length heavy chain amino acid sequence is SEQ ID NO: 178 (heavy chain nucleic acid sequence SEQ ID NO: 179). A full-length light chain amino acid sequence is SEQ ID NO: 182 (light chain nucleic acid sequence SEQ ID NO: 183). The heavy chain variable region is made by recombination of human IGHV3-23*04 (SEQ ID NO: 312), IGHD1-20*01 (SEQ ID NO: 316) and IGHJ6*02 (SEQ ID NO: 318) gene segments. The light chain variable region is made by recombination of human IGKV1-17*01 (SEQ ID NO: 320) and IGKJ4*01 (SEQ ID NO: 326) gene segments.

CL-82517 has a heavy chain variable region (V_{H}) amino acid sequence of SEQ ID NO: 184, comprising a CDRH1, CDRH2 and CDRH3 amino acid sequences as defined by IMGT or Kabat from V_{H} amino acid sequence SEQ ID NO: 184. The heavy chain nucleic acid sequence of the V_{H} domain is SEQ ID NO: 185. CL-82517 has a light chain variable region (V_{L}) amino acid sequence of SEQ ID NO: 188, comprising the CDRL1, CDRL2 and CDRL3 amino acid sequences as defined by IMGT or Kabat from V_{L} amino acid sequence of SEQ ID NO: 188. The light chain nucleic acid sequence of the V_{L} domain is SEQ ID NO: 189. The V_{H} domain may be combined with any of the heavy chain constant region sequences described herein, e.g. SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 267, SEQ ID NO: 269, SEQ ID NO: 305 or SEQ ID NO: 307. The V_{L} domain may be combined with any of the light chain constant region sequences described herein, e.g. SEQ ID NO: 271, SEQ ID NO: 273, SEQ ID NO: 275, SEQ ID NO: 277, SEQ ID NO: 279, SEQ ID NO: 281, SEQ ID NO: 284, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301 or SEQ ID NO: 303. A full-length heavy chain amino acid sequence is SEQ ID NO: 186 (heavy chain nucleic acid sequence SEQ ID NO: 187). A full-length light chain amino acid sequence is SEQ ID NO: 190 (light chain nucleic acid sequence SEQ ID NO: 191). The heavy chain variable region is made by recombination of human IGHV3-73*02 (SEQ ID NO: 309), IGHD3-9*01 (SEQ ID NO: 314) and IGHJ6*02 (SEQ ID NO: 318) gene segments. The light chain variable region is made by recombination of human IGKV1-5*03 and IGKJ1*01 (SEQ ID NO: 325) gene segments.

CL-82495 has a heavy chain variable region (V_{H}) amino acid sequence of SEQ ID NO: 192, comprising a CDRH1, CDRH2 and CDRH3 amino acid sequences as defined by IMGT or Kabat from V_{H} amino acid sequence SEQ ID NO: 192. The heavy chain nucleic acid sequence of the V_{H} domain is SEQ ID NO: 193. CL-82495 has a light chain variable region (V_{L}) amino acid sequence of SEQ ID NO: 196, comprising the CDRL1, CDRL2 and CDRL3 amino acid sequences as defined by IMGT or Kabat from V_{L} amino acid sequence of SEQ ID NO: 196. The light chain nucleic acid sequence of the V_{L} domain is SEQ ID NO: 197. The V_{H} domain may be combined with any of the heavy chain constant region sequences described herein, e.g. SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 267, SEQ ID NO: 269, SEQ ID NO: 305 or SEQ ID NO: 307. The V_{L} domain may be combined with any of the light chain constant region sequences described herein, e.g. SEQ ID NO: 271, SEQ ID NO: 273, SEQ ID NO: 275, SEQ ID NO: 277, SEQ ID NO: 279, SEQ ID NO: 281, SEQ ID NO: 284, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301 or SEQ ID NO: 303. A full-length heavy chain amino acid sequence is SEQ ID NO: 194 (heavy chain nucleic acid sequence SEQ ID NO: 195). A full-length light chain amino acid sequence is SEQ ID NO: 198 (light chain nucleic acid sequence SEQ ID NO: 199). The heavy chain variable region is made by recombination of human IGHV3-33*01 (SEQ ID NO: 311), IGHD3-10*01 (SEQ ID NO: 315) and IGHJ6*02 (SEQ ID NO: 318) gene segments. The light chain variable region is made by recombination of human IGKV1-17*01 (SEQ ID NO: 320) and IGKJ1*01 (SEQ ID NO: 325) gene segments.

CL-82473 has a heavy chain variable region (V_{H}) amino acid sequence of SEQ ID NO: 200, comprising a CDRH1, CDRH2 and CDRH3 amino acid sequences as defined by IMGT or Kabat from V_{H} amino acid sequence SEQ ID NO: 200. The heavy chain nucleic acid sequence of the V_{H} domain is SEQ ID NO: 201. CL-82473 has a light chain variable region (V_{L}) amino acid sequence of SEQ ID NO: 204, comprising the CDRL1, CDRL2 and CDRL3 amino acid sequences as defined by IMGT or Kabat from V_{L} amino acid sequence of SEQ ID NO: 204. The light chain nucleic acid sequence of the V_{L} domain is SEQ ID NO: 205. The V_{H} domain may be combined with any of the heavy chain constant region sequences described herein, e.g. SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 267, SEQ ID NO: 269, SEQ ID NO: 305 or SEQ ID NO: 307. The V_{L} domain may be combined with any of the light chain constant region sequences described herein, e.g. SEQ ID NO: 271, SEQ ID NO: 273, SEQ ID NO: 275, SEQ ID NO: 277, SEQ ID NO: 279, SEQ ID NO: 281, SEQ ID NO: 284, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301 or SEQ ID NO: 303. A full-length heavy chain amino acid sequence is SEQ ID NO: 202 (heavy chain nucleic acid sequence SEQ ID NO: 203). A full-length light chain amino acid sequence is SEQ ID NO: 206 (light chain nucleic acid sequence SEQ ID NO: 207). The heavy chain variable region is made by recombination of human IGHV3-33*01 (SEQ ID NO: 311), IGHD3-10*01 (SEQ ID NO: 315) and IGHJ4*02 gene segments. The light chain variable region is made by recombination of human IGKV2-30*01 and IGKJ3*01 (SEQ ID NO: 323) gene segments.

CL-82455 has a heavy chain variable region (V_{H}) amino acid sequence of SEQ ID NO: 208, comprising a CDRH1, CDRH2 and CDRH3 amino acid sequences as defined by IMGT or Kabat from V_{H} amino acid sequence SEQ ID NO: 208. The heavy chain nucleic acid sequence of the V_{H} domain is SEQ ID NO: 209. CL-82455 has a light chain variable region (V_{L}) amino acid sequence of SEQ ID NO: 212, comprising the CDRL1, CDRL2 and CDRL3 amino acid sequences as defined by IMGT or Kabat from V_{L} amino acid sequence of SEQ ID NO: 212. The light chain nucleic acid sequence of the V_{L} domain is SEQ ID NO: 213. The V_{H} domain may be combined with any of the heavy chain constant region sequences described herein, e.g. SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 267, SEQ ID NO: 269, SEQ ID NO: 305 or SEQ ID NO: 307. The V_{L} domain may be combined with any of the light chain constant region sequences described herein, e.g. SEQ ID NO: 271, SEQ ID NO: 273, SEQ ID NO: 275, SEQ ID NO: 277, SEQ ID NO: 279, SEQ ID NO: 281, SEQ ID NO: 284, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301 or SEQ ID NO: 303. A full-length heavy chain amino acid sequence is SEQ ID NO: 210 (heavy chain nucleic acid sequence SEQ ID NO: 211). A full-length light chain amino acid sequence is SEQ ID NO: 214 (light chain nucleic acid sequence SEQ ID NO: 215). The heavy chain variable region is made by recombination of human IGHV3-33*01 (SEQ ID NO: 311), IGHD3-10*01 (SEQ ID NO: 315) and IGHJ6*02 (SEQ ID NO: 318) gene segments. The light chain variable region is made by recombination of human IGKV3-15*01 and IGKJ1*01 (SEQ ID NO: 325) gene segments.

CL-83158 has a heavy chain variable region (V_{H}) amino acid sequence of SEQ ID NO: 216, comprising a CDRH1, CDRH2 and CDRH3 amino acid sequences as defined by IMGT or Kabat from V_{H} amino acid sequence SEQ ID NO: 216. The heavy chain nucleic acid sequence of the V_{H} domain is SEQ ID NO: 217. CL-83158 has a light chain variable region (V_{L}) amino acid sequence of SEQ ID NO: 220, comprising the CDRL1, CDRL2 and CDRL3 amino acid sequences as defined by IMGT or Kabat from V_{L} amino acid sequence of SEQ ID NO: 220. The light chain nucleic acid sequence of the V_{L} domain is SEQ ID NO: 221. The V_{H} domain may be combined with any of the heavy chain constant region sequences described herein, e.g. SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 267, SEQ ID NO: 269, SEQ ID NO: 305 or SEQ ID NO: 307. The V_{L} domain may be combined with any of the light chain constant region sequences described herein, e.g. SEQ ID NO: 271, SEQ ID NO: 273, SEQ ID NO: 275, SEQ ID NO: 277, SEQ ID NO: 279, SEQ ID NO: 281, SEQ ID NO: 284, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301 or SEQ ID NO: 303. A full-length heavy chain amino acid sequence is SEQ ID NO: 218 (heavy chain nucleic acid sequence SEQ ID NO: 219). A full-length light chain amino acid sequence is SEQ ID NO: 222 (light chain nucleic acid sequence SEQ ID NO: 223). The heavy chain variable region is made by recombination of human IGHV4-34*01, IGHD3-10*01 (SEQ ID NO: 315) and IGHJ6*02 (SEQ ID NO: 318) gene segments. The light chain variable region is made by recombination of human IGLV3-19*01 and IGLJ3*02 (SEQ ID NO: 324) gene segments.

CL-148712 has a heavy chain variable region (V_{H}) amino acid sequence of SEQ ID NO: 224, comprising a CDRH1, CDRH2 and CDRH3 amino acid sequences as defined by IMGT or Kabat from V_{H} amino acid sequence SEQ ID NO: 224. The heavy chain nucleic acid sequence of the V_{H} domain is SEQ ID NO: 225. CL-148712 has a light chain variable region (V_{L}) amino acid sequence of SEQ ID NO: 228, comprising the CDRL1, CDRL2 and CDRL3 amino acid sequences as defined by IMGT or Kabat from V_{L} amino acid sequence of SEQ ID NO: 228. The light chain nucleic acid sequence of the V_{L} domain is SEQ ID NO: 229. The V_{H} domain may be combined with any of the heavy chain constant region sequences described herein, e.g. SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 267, SEQ ID NO: 269, SEQ ID NO: 305 or SEQ ID NO: 307. The V_{L} domain may be combined with any of the light chain constant region sequences described herein, e.g. SEQ ID NO: 271, SEQ ID NO: 273, SEQ ID NO: 275, SEQ ID NO: 277, SEQ ID NO: 279, SEQ ID NO: 281, SEQ ID NO: 284, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301 or SEQ ID NO: 303. A full-length heavy chain amino acid sequence is SEQ ID NO: 226 (heavy chain nucleic acid sequence SEQ ID NO: 227). A full-length light chain amino acid sequence is SEQ ID NO: 230 (light chain nucleic acid sequence SEQ ID NO: 231). The heavy chain variable region is made by recombination of human IGHV3-23*04 (SEQ ID NO: 312), IGHD1-1*01 (SEQ ID NO: 317) and IGHJ6*02 (SEQ ID NO: 318) gene segments. The light chain variable region is made by recombination of human IGKV1-17*01 (SEQ ID NO: 320) and IGKJ4*01 (SEQ ID NO: 326) gene segments.

CL-148729 has a heavy chain variable region (V_{H}) amino acid sequence of SEQ ID NO: 232, comprising a CDRH1, CDRH2 and CDRH3 amino acid sequences as defined by IMGT or Kabat from V_{H} amino acid sequence SEQ ID NO: 232. The heavy chain nucleic acid sequence of the V_{H} domain is SEQ ID NO: 233. CL-148729 has a light chain variable region (V_{L}) amino acid sequence of SEQ ID NO: 236, comprising the CDRL1, CDRL2 and CDRL3 amino acid sequences as defined by IMGT or Kabat from V_{L} amino acid sequence of SEQ ID NO: 236. The light chain nucleic acid sequence of the V_{L} domain is SEQ ID NO: 237. The V_{H} domain may be combined with any of the heavy chain constant region sequences described herein, e.g. SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 267, SEQ ID NO: 269, SEQ ID NO: 305 or SEQ ID NO: 307. The V_{L} domain may be combined with any of the light chain constant region sequences described herein, e.g. SEQ ID NO: 271, SEQ ID NO: 273, SEQ ID NO: 275, SEQ ID NO: 277, SEQ ID NO: 279, SEQ ID NO: 281, SEQ ID NO: 284, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301 or SEQ ID NO: 303. A full-length heavy chain amino acid sequence is SEQ ID NO: 234 (heavy chain nucleic acid sequence SEQ ID NO: 235). A full-length light chain amino acid sequence is SEQ ID NO: 238 (light chain nucleic acid sequence SEQ ID NO: 239). The heavy chain variable region is made by recombination of human IGHV3-73*02 (SEQ ID NO: 309), IGHD6-25*01 and IGHJ4*02 gene segments. The light chain variable region is made by recombination of human IGLV1-51*01 and IGLJ3*02 (SEQ ID NO: 324) gene segments.

Antibodies or antigen-binding fragments of the invention (as described herein) specifically bind to CXCR4; specific binding and/or binding affinity to CXCR4 may be determined by SPR or other techniques known in the field. In one embodiment, an antibody or antigen-binding fragment specifically binds to human CXCR4. In a preferred embodiment, am antibody or antigen-binding fragment specifically binds to both human CXCR4 and cynomolgus CXCR4.

In one embodiment, the SPR is carried out using nanodiscs, cells or virus-like particles (VLP) to present all or part of the receptor (e.g. CXCR4). In another embodiment, nanodiscs, cells or VLP are used to present a mutant (e.g. thermostabilised) or variant receptor (e.g. CXCR4). In one embodiment, the SPR is carried out using a conformationally thermo-stabilised receptor, for example a CXCR4 StaR®, which is mutant (thermostabilised) CXCR4 receptor which has be generated and selected according to the methods described in WO2009/081136 (see also: Magnani et al (2008). Co-evolving stability and conformational homogeneity of the human adenosine A₂ₐ receptor. Proc Natl Acad Sci USA. 105(31):10744-10749; Serrano-Vega et al (2008). Conformational thermostabilization of the beta1-adrenergic receptor in a detergent-resistant form. Proc Natl Acad Sci USA. 105(3):877-882; and Shibata et al(2009). Thermostabilization of the neurotensin receptor NTS1. J Mol Biol. 390(2):262-277) which is optionally presented using nanodiscs, cells or VLPs.

In one embodiment, the SPR is carried out at 25° C. or 37° C. In one embodiment, the SPR is carried out at physiological pH, such as about pH 7 or at pH 7.6 (e.g., using Hepes buffered saline at pH 7.6 (also referred to as HBS-EP, available from Teknova Inc (California; catalogue number H8022)). In one embodiment, the SPR is carried out at a physiological salt level, e.g., 150 mM NaCl. In one embodiment, the SPR is carried out at a detergent level of no greater than 0.05% by volume, e.g., in the presence of P20 (polysorbate 20; e.g., Tween-20™) at 0.05% and EDTA at 3 mM.

In one example, the SPR is carried out at 25° C. and using 10 nM Hepes, 150 mM NaCl, 0.1% w/v BSA as running buffer at pH 7.3. In one example, the SPR is carried out at 25° C. or 37° C. in a buffer at pH 7.6, 150 mM NaCl, 0.05% detergent (e.g., P20) and 3 mM EDTA. The buffer can contain 10 mM Hepes. In one example, the SPR is carried out at 25° C. or 37° C. in HBS-EP. In an example, the affinity of the antibody or antigen-binding fragment is determined using SPR by:
1. Coupling anti-human (or other relevant human, rat or non-human vertebrate antibody constant region species-matched) IgG (e.g., Biacore™ BR-1008-38) to a biosensor chip (e.g., GLM chip) such as by primary amine coupling;
2. Exposing the anti-human IgG (or other matched species antibody) to a test IgG antibody (e.g. anti-CXCR4 antibody or antigen-binding fragment) to capture test antibody on the chip;
3. Passing the test antigen over the chip's capture surface at 1024 nM, 256 nM, 64 nM, 16 nM, 4 nM with a 0 nM (i.e. buffer alone); and
4. And determining the affinity of binding of test antibody to test antigen using surface plasmon resonance, e.g., under an SPR condition discussed above (e.g. at 25° C. in physiological buffer). SPR can be carried out using any standard SPR apparatus, such as by Biacore™ or using the ProteOn XPR36™ (Bio-Rad™)
In another embodiment, antigen can be presented on a biosensor chip (instead of step 1 and 2) and then said antigen is exposed to an antibody or antigen-binding fragment (instead of chip 3). In another embodiment, biotinylated CXCR4-virus like particle were captured on the active flow channel of a SA chip and then purified anti-CXCR4 antibodies or antigen-binding fragments (e.g. Fab fragment) were used as analytes and flown over captured CXCR4 VLP at 1.23 nM, 3.7 nM, 11.11 nM, 33.33 nM and 100 nM.

In another embodiment, SPR carried out using cells can be analysed using technology which measures repeated differential measurements of surface associated proteins (e.g. LigandTracer™ technology (Ridgeview)).

Regeneration of the capture surface can be carried out with 10 mM glycine at pH 1.7. This removes the captured antibody or antigen-binding fragment and allows the surface to be used for another interaction. The binding data can be fitted to 1:1 model inherent using standard techniques, e.g., using a model inherent to the ProteOn XPR36™ analysis software.

An antibody or antigen-binding fragment thereof that specifically binds to a CXCR4 antigen may be cross-reactive with related antigens, in particular CXCR4 of a different species, e.g., rhesus, cynomolgus, rodent and/or human. Thus, in one embodiment, the antibody or antigen-binding fragment specifically binds to human, rhesus, cynomolgus and/or rodent (e.g. mouse or rat) CXCR4. In one embodiment, the antibody or antigen-binding fragment specifically binds to human and/or cynomolgus CXCR4.

In a preferred embodiment, an antibody or an antigen-binding fragment thereof that specifically binds to a human CXCR4 antigen does not cross-react with other antigens, for example an antigen selected from CXCR1, CXCR2, CXCR3, CXCR5, CXCR6, CXCR7, CXCR8, CXCR9, CXCR10, CCR1, CCR2, CCR3, CCR4, CCR5, CCR6, CCR7, CCR8, CCR9, CCR10 and CB2, or any combination thereof. In one embodiment, the antibody or antigen-binding fragment does not bind to or cross-react with CXCR7, e.g. does not detectably bind to CXCR7 (optionally wherein CXCR7 is human and is further optionally selected from SEQ ID NO: 4, SEQ ID NO: 5 or SEQ ID NO: 6). In a preferred embodiment, the antibody or antigen-binding fragment does not bind to CXCR7 (optionally wherein CXCR7 is human and is further optionally selected from SEQ ID NO: 4, SEQ ID NO: 5 or SEQ ID NO: 6) when compared to a positive control (e.g. an anti-CXCR7 antibody). In another embodiment, the antibody or antigen-binding fragment does not bind to or cross-react with CCR5, e.g. does not detectably bind to CCR5. In another embodiment, the antibody or antigen-binding fragment does not bind to or cross-react with CXCR3, e.g. does not detectably bind to CXCR3. In one embodiment, cross-reactivity is determined essentially as described in Example 12 herein.

In one embodiment, the antibody or antigen-binding fragment specifically binds to CXCR4 (e.g. human CXCR4). In one embodiment, specific binding can be measure by binding affinity, which may be expressed as either EC₅₀ or K_{D}. In one embodiment, the antibody or antigen-binding fragment specifically binds to human CXCR4 with a K_{D} of from 0.2 to 2 nM, from 0.2 to 1 nM, from 0.2 to 0.8 nM, or preferably from 0.4 to 0.8 nM. In one embodiment, the binding affinity is determined by SPR, for example as described elsewhere herein or carried out essentially as described in Example 11 herein.

In another embodiment, the antibody or antigen-binding fragment specifically binds to cynomolgus CXCR4 with an EC₅₀ of from 0.5 to 10 nM, from 0.5 to 8 nM, or preferably from 0.5 to 5 nM. In one embodiment, the binding affinity is determined by flow cytometry for example as described elsewhere herein or carried out essentially as described in Example 10 herein.

In one embodiment, the antibody or antigen-binding fragment (substantially) inhibits the binding of a ligand (e.g. native ligand as described above) to CXCR4. In a preferred embodiment, the ligand is CXCL12 (e.g. human CXCL12, in particular human alpha CXCL12 (SEQ ID NO: 327)). In one embodiment, CXCL12 has a signal peptide which is cleaved (optionally 21 amino acids in length). In a preferred embodiment, the antibody or antigen-binding fragment (substantially) inhibits the binding of CXCL12 to CXCR4. In one embodiment, the antibody or antigen-binding fragment completely inhibits the binding of CXCL12 to CXCR4 (e.g. human CXCL12 to human CXCR4 and/or cynomolgus CXCL12 to cynomolgus CXCR4). In one embodiment, the antibody or antigen-binding fragment partially inhibits the binding of CXCL12 to CXCR4. In one embodiment, the antibody or antigen-binding fragment partially or completely inhibits binding of CXCL12 to CXCR4, but does not show any detectable inhibition of the binding of other CXCR4 ligands (e.g. MIF or ubiquitin) to CXCR4.

In a preferred embodiment, the antibody or antigen-binding fragment inhibits the binding of CXCL12 (e.g. human or cynomolgus CXCL12) to CXCR4 (e.g. human or cynomolgus CXCR4) with an IC₅₀ from 0.2 to 4 nM, from 0.2 to 3 nM, from 0.2 to 2 nM, or preferably from 0.2 to 1 nM. In a preferred embodiment, the antibody or antigen-binding fragment inhibits the binding of human CXCL12 to human CXCR4 (optionally selected from SEQ ID NO: 1, SEQ ID NO: 2 or SEQ ID NO: 3) with an IC₅₀ of from 0.4 to 4 nM. In a preferred embodiment, CXCL12 inhibition is determined using a HTRF assay as described elsewhere herein or carried out essentially as described in Example 7 herein.

In one embodiment, the HTRF assay is carried out at room temperature (e.g. 22 or 25° C.). In another embodiment, the assay is performed in the dark, in particular during incubation periods. In any HTRF assay, appropriate donor-accepter fluorescent pairs are used. The donor molecule is conjugated to either the receptor of interest (i.e. CXCR4) or to a ligand of interest (e.g. CXCL12). The acceptor molecule is conjugated to the binding partner of the receptor/ligand to which the donor molecule is conjugated. The donor molecule or acceptor molecule may be linked to e.g. anti-mouse Fc antibody, anti-human Fc antibody, anti-Flag antibody, anti-His antibody, directly to streptavidin [each of which is conjugated to either the ligand or receptor of interest (e.g. CXCL12 or CXCR4)], or directly to the ligand or receptor of interest (e.g. CXCL12 or CXCR4). In one example, the donor molecule is cryptate (620 nm emission) and the acceptor molecule is selected from D2 (647 nm emission) and Alexaflour647 (emission 665 nm). In one embodiment, the concentration of antibody or antigen-binding fragment thereof is titrated to provide a curve of % specific binding (see Example 7, Equation 6). In one embodiment, the concentration of antigen or antigen-binding fragment ranges from approximately 1 mM to 1 pM (e.g. 500 nM to 15 pM or 50 nM to 5 pM). In another embodiment, a plurality of data points are taken, e.g. 6, 7, 8, 9, 10, 11, 12, or 13 data points, in particular 10 or 11 data points. In one embodiment, the HTRF assay is carried out in HTRF buffer (e.g. PBS (Sigma)+0.53 M KF (Sigma)+0.1% w/v BSA (Sigma)).

In an example, the % specific binding of the ligand (e.g. CXCL12) is determined using HTRF by:
1. Combining recombinant ligand (e.g. CXCL12 (tagged with AlexaFlour647)) with antibody or antigen-binding fragment in a plate;
2. Adding cells expressing receptor (e.g. CXCR4 (SNAP-tagged with europium cryptate)) to the plate;
3. Adding test antibody or antigen-binding fragment and leaving for a sufficient period of time to equilibrate (e.g. approximately 1 or 2 h at room temperature);
4. Reading the plate using a reader capable of exciting the donor molecule (e.g. at 335 nm) and capable of measuring the wavelength of emission of both the donor and accepter fluorescent molecules (e.g. at 620 nm and 665 nm) (Examples of plate readers include EnVision™ (Perkin Elmer), PHERAstar™ FS, PHERAstar™ FSX, CLARIOstar™, and FLUOstar™, and POLARstar™ Omega); and
5. Determining the % specific binding of the test molecule (e.g. CXCL12), for example using Equation 6; and/or
6. Determining the IC₅₀ of the ligand (e.g. CXCL12), for example using Equation 5. In one embodiment, the (e.g. inhibition) data (e.g. % specific binding) can be fitted using standard analysis techniques, e.g., using GraphPad/PRISM analysis software.

In one embodiment, the anti-CXCR4 antibodies or antigen-binding fragments described herein do not induce apoptosis (e.g. CXCR4-mediated apoptosis) in T-cells (optionally CD8⁺ T-cells), optionally wherein apoptosis is determined using flow cytometry. In one embodiment, the anti-CXCR4 antibodies or antigen-binding fragments described herein do not induce CXCR4-mediated apoptosis (either by direct effect on CXCR4 or through the effect of inhibiting one or more of its native ligands), for example in cells expressing CXCR4 in vitro, optionally determined using flow cytometry. In one embodiment, apoptosis is determined using Jurkat T-cells or primary naïve T-cells (e.g. mammalian or human). In one embodiment, the T-cells are CXCR4-expressing T-cells. The T-cells may be CD8⁺ T-cells, CD4⁺ T-cells or regulatory T-cells (Tregs), preferably CD8⁺ T-cells. In one embodiment, a suitable isotype control (e.g. IgG4-PE) and positive control (e.g. anti-Fas antibody or Staurosporine) are used in the assay for determining apoptosis. In one embodiment, the antibody or antigen-binding fragment does not induce apoptosis in T cells if the antibody or fragment has a maximal percentage specific-induced apoptosis (optionally expressed as a percentage of anti-Fas response or Staurosporine-induced apoptosis) of less than 50%, less than 40%, less than 30% or less than 20%. In another embodiment, the maximal percentage specific-induced apoptosis (optionally expressed as a percentage of anti-Fas response or Staurosporine-induced apoptosis) is less than 10%, less than 8%, less than 5% or less than 4%, in particular less than 5%. In another embodiment, the maximal percentage specific-induced apoptosis (optionally expressed as a percentage of anti-Fas response or Staurosporine-induced apoptosis) is less than 3%, less than 2%, less than 1% or less than 0.8%. In another embodiment, the maximal percentage specific-induced apoptosis (optionally w expressed as a percentage of anti-Fas response or Staurosporine-induced apoptosis) is less than 0.5%, less than 0.3%, less than 0.2% or less than 0.1%. In a preferred embodiment, the maximal percentage specific-induced apoptosis is determined using flow cytometry for example as described herein or carried out essentially as described in Example 9A or 9B herein. In another embodiment, the antibody (for example an antibody have reduced or no effector function) or antigen-binding fragment does not induce CXCR4-mediated apoptosis of B-cells.

Apoptosis is a programmed form of cell death involving the degradation of cellular constituents by a group of cysteine proteases called caspases. The caspases can be activated through either the intrinsic (mitochondrial-mediated) or extrinsic (e.g. death receptor- and effector cell-mediated) apoptotic pathways. Examples of extrinsic activation could be by Fas activation or the release of granzymes and perforins from effector cells. In one embodiment, the anti-CXCR4 antibodies or antigen-binding fragments described herein do not induce extrinsic (e.g. death receptor- and effector cell-mediated) and/or intrinsic (mitochondrial-mediated) apoptosis.

In one embodiment, the anti-CXCR4 antibodies or antigen-binding fragments described herein (substantially) inhibit CXCL12 (e.g. human or cynomolgus CXCL12) signalling via CXCR4 (e.g. human or cynomolgus CXCR4), for example inhibiting CXCL12-mediated CXCR4 signalling via cAMP. In one embodiment, the antibody or antigen-binding fragment (substantially) inhibits CXCL12-mediated inhibition of forskolin-stimulated cAMP, for example using a forskolin-stimulated cAMP signalling assay (e.g. AlphaScreen™ cAMP assay). In one embodiment, the antibody or antigen-binding fragment completely or substantially inhibits CXCL12-mediated inhibition of forskolin-stimulated cAMP. In another embodiment, the antibody or antigen-binding fragment partially inhibits CXCL12-mediated inhibition of forskolin-stimulated cAMP. In one embodiment, the antibody or antigen-binding fragment inhibits CXCL12-mediated inhibition of forskolin-stimulated cAMP with an IC₅₀ of from 1 to 100 nM, from 1 to 90 nM, from 1 to 80 nM, from 1 to 70 nM, from 1 to 60 nM, from 1 to 50 nM, from 1 to 40 nM, or preferably from 1 to 30 nM. In one embodiment, cAMP is determined using a forskolin-stimulated cAMP signalling assay, which is carried out essentially as described in Example 6 (human CXCR4) and Example 10B (cynomolgus CXCR4) herein.

In one embodiment, the anti-CXCR4 antibodies or antigen-binding fragments described herein (substantially) inhibit beta-arrestin recruitment to CXCR4 (e.g. hCXCR4 or cynomolgus CXCR4), optionally wherein said beta-arrestin recruitment is induced by CXCL12 (e.g. hCXCL12 or cynomolgus CXCL12). In one embodiment, the antibody or antigen-binding fragment inhibits beta-arrestin recruitment to CXCR4 with an IC₅₀ of from 0.01 to 15 nM, from 0.01 to 10 nM, from 0.01 to 5 nM, from 0.01 to 4 nM, from 0.01 to 3 nM, from 0.01 to 2 nM, from 0.01 to 1.5 nM, from 0.01 to 1 nM, from 0.01 to 0.5 nM, or preferably from 0.01 to 0.2 nM. In one embodiment, inhibition of beta-arrestin recruitment to CXCR4 is performed in a functional cell-based reporter gene assay (e.g. using Tango CXCR4-bla U2OS cells). In one embodiment, the inhibition of beta-arrestin recruitment is measured using 30 nM of CXCL12 to induce beta-arrestin. In one embodiment, the concentration of antibody or antigen-binding fragment is from 100 nM to 0.1 pM (e.g. 50 nM to 0.5 pM or 40 nM to 0.5 pM). In one embodiment, functional cell-based reporter gene assay is carried out essentially as described in Example 6C herein.

In one embodiment, the anti-CXCR4 antibodies or antigen-binding fragments described herein (significantly) internalise into the cell upon binding to the cell surface, for example upon binding to CXCR4 on the cell surface, and optionally wherein internalisation is determined using fluorescent microscopy. In one embodiment, the antibody or antigen-binding fragment is internalised into the cell upon binding to CXCR4 on the cell surface with a maximum signal of total overlap object area of from 1.2-fold to 10-fold, from 1.2-fold to 5-fold, from 1.2-fold to 4-fold, from 1.2-fold to 3-fold or from 1.2-fold to 2-fold greater than an isotype control. In one embodiment, the maximum signal of total overlap object area is from 2-fold to 10-fold, from 2-fold to 5-fold, from 2-fold to 4-fold, from 2-fold to 3-fold, from 3-fold to 4-fold or from 3-fold to 5-fold greater than an isotype control, in particular from 2-fold to 4-fold. In one embodiment, the isotype control is IgG4-PE. In one embodiment, internalisation is determined using fluorescent imaging techniques (e.g. IncuCyte™ S3 Live Cell Analysis System (Essen BioScience)) or ELISA, in particular fluorescent imaging techniques. In one embodiment, internalisation of anti-CXCR4 antibodies or antigen-binding fragments is determined essentially as described in Example 8 herein.

In one embodiment, fluorescent imaging techniques comprise Live-cell analysis, which measures real-time quantification of live-cell imaging and analysis. This technique enables visualisation and quantification of cell behaviour over time and optionally automatically gathers and analyses images in a laboratory incubator. An example of a live-cell analysis system is the IncuCyte™ S3 Live Cell Analysis System (Essen BioScience), which is used for measuring receptor or antibody (or antigen-binding fragment) internalisation, chemotaxis, apoptosis or cell infiltration into 3D cell environment (e.g. tumour Spheroid). Internalisation results in a positive readout of secondary antibody (red fluorescence).

Without being bound by theory, the antibodies or antigen-binding fragments of the invention may be internalised and recycled back to the cell surface along with the receptor (e.g. CXCR4). Alternatively, they may remain bound to the receptor inside the cell (e.g. CXCR4), which prevents them from being degraded by the usual cellular mechanisms. Alternatively, the antibody or antigen-binding fragment bound to the receptor is internalised and the receptor is recycled back to the cell surface without the antibody or antigen-binding fragment. Because the antibodies or antigen-binding fragments of the invention are not internalised as rapidly as benchmark antibodies, the inventors postulate that they remain bound to receptor on the cell surface for longer, inhibiting the ligand (e.g. CXCL12) for longer, and therefore the antibodies or antigen-binding fragments of the invention may have higher dissociation rate constant (Kd) values, even though the overall affinity appears to be similar. An alternative theory is that the antibodies or antigen-binding fragments of the invention bind to monomeric CXCR4 and a single antibody or antigen-binding fragment does not bind to more than one CXCR4 protein (monomer) due to steric hindrance. The antibodies or antigen-binding fragments of the invention therefore may not cross-link two receptors and as a result internalisation of the antibody or antigen-binding fragment:receptor complex is slower.

In one embodiment, the anti-CXCR4 antibodies or antigen-binding fragments described herein (substantially) inhibit ligand-mediated (e.g. CXCL12, MIF and/or ubiquitin, in particular CXCL12) response of T-cells, for example as measured by alteration of the shape of the cells. In such an assay, the observed change of shape of the cells is caused by either a redistribution of cellular matter, or by a change in the cell membrane, or by a change in the location of intracellular components. In one embodiment, the ligand-mediated response of T-cells is determined using electrical conduction or is determined using label free dynamic mass redistribution (DMR) assay. In one embodiment, the antibody or antigen-binding fragment inhibits CXCL12 mediated response of T-cells with an IC₅₀ of from 0.5 to 20 nM, from 0.5 to 15 nM, from 0.5 to 13 nM, from 0.15 to 10 nM, from 0.5 to 8 nM, or preferably from 0.5 to 5 nM. In one embodiment, DMR assay is carried out essentially as described in Example 13 herein.

In one embodiment, the anti-CXCR4 antibodies or antigen-binding fragments described herein (substantially) inhibit chemotaxis of CXCR4⁺ T-cells (e.g. human T-cells, such as primary human T-cells extracted from human blood) to a CXCR4 ligand, such as CXCL12. In one embodiment, chemotaxis is determined using live cell imaging (e.g. IncuCyte™ S3 chemotaxis assay system) or flow cytometry (e.g. a transwell based flow cytometry method), optionally using Jurkat T-cells. In one embodiment 30 nM or 6.25 nM of CXCL12 is used to induce chemotaxis (as the chemoattractant) in the assay. In one embodiment, the antibody or antigen-binding fragment inhibits chemotaxis of CXCR4⁺ T-cells to CXCL12 with an IC₅₀ of from 0.01 to 5 nM, from 0.01 to 3 nM, from 0.01 to 2 nM, or preferably from 0.01 to 1 nM. In one embodiment, chemotaxis assay is carried out essentially as described in Example 14 herein.

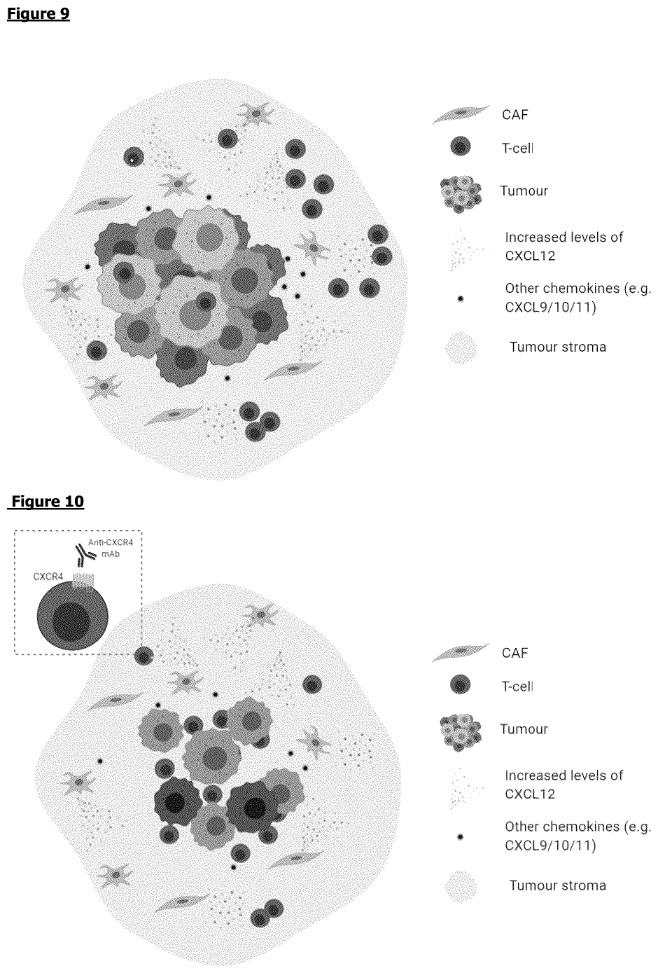

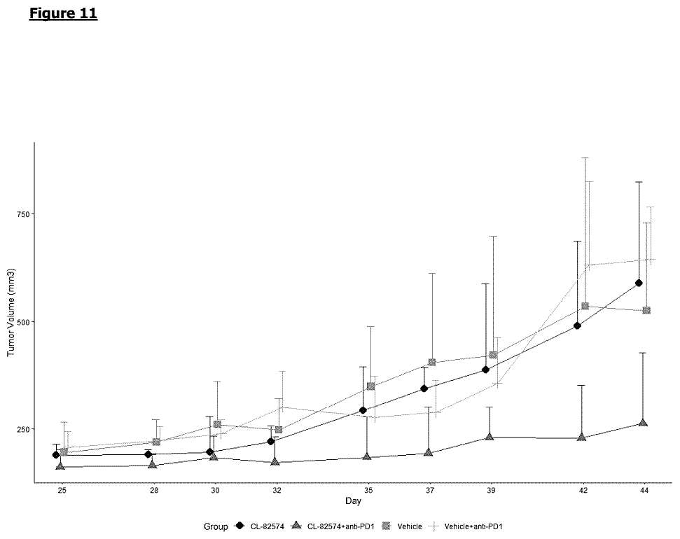

In one embodiment, the anti-CXCR4 antibodies or antigen-binding fragments described herein (substantially or significantly) enable T-cells (e.g. CD8⁺ T-cells) to infiltrate (move into) a tumour (e.g. a solid tumour), optionally as measured in a tumour spheroid assay. Tumour infiltration can be measured in vivo by assessing T-cell infiltration into a tumour in a mouse (by comparing samples from an untreated mouse and a mouse treated with anti-CXCR4 antibodies or antigen-binding fragments described herein) using FACS or immunohistochemistry (IHC) (taking multiple cross-sections of the treated and untreated tumours, applying a reagent which identifies T-cells and comparing the two). A tumour (e.g. central tumour mass) is surrounded by tumour stroma (see FIGS. 9 and 10). In a preferred embodiment, anti-CXCR4 antibodies or antigen-binding fragments described herein (substantially or significantly) enable T-cells (e.g. CD8⁺ T-cells) to infiltrate the tumour from the tumour stroma. Once the T-cells are within the tumour (e.g. central tumour mass), they are able to effect cell-killing and can destroy the malignant cells. A potential mechanism for this is shown in FIG. 10. Without being bound by theory, in the absence of the anti-CXCR4 antibodies described herein, the cancer associated fibroblasts (CAFs) which are present in the stroma, secrete CXCL12 and create a stronger gradient away from the tumour cells, which the T-cells preferentially migrate towards. This is depicted schematically in FIG. 9.

Tumour infiltration can be measured in vitro using a spheroid assay. Spheroids are 3D cell culture models which can be used to mimic some features of solid tumours. 3D cell culture offers many benefits over 2D cell culture, for example improved cell-to-cell contact which is more representative of a tumour and creates tumour environmental conditions (e.g. hypoxia and necrosis). Tumour spheroids can be used as in vitro models for screening therapeutics (Costa et al., (2016). 3D tumor spheroids: an overview on the tools and techniques used for their analysis. Biotechnol Adv. 34(8):1427-1441). The spheroid contains tumour cells (e.g. HT-29 cells) which secrete CXCR3 ligands (CXCL9, CXCL10 and CXCL11) when stimulated with interferon gamma (IFNγ). Recombinant CXCL12 is added to culture media to represent CXCL12 found within the tumour stroma. The concentration added is large enough to outweigh the tumour-secreted CXCL9, CXCL10 and CXCL11 pro-infiltrating chemokine gradients, and thus more accurately reflects in vivo conditions. The assay uses CXCR4 expressing CCRF-HSB-2 cells, which are T-cell lymphoblasts, and mimic T-cells in a tumour environment. It is thought that inhibition of the CXCL12 signalling by anti-CXCR4 antibodies results in the restoration of the CXCR3 chemotaxis of T-cell lymphoblasts towards CXCL9, CXCL10 and/or CXCL11 released by the spheroids and resulted in increased levels of infiltration of the T-cells allowing reduction in tumour volume. Thus, in one embodiment, the anti-CXCR4 antibodies or antigen-binding fragments described herein (significantly) induce an increase in T-cell lymphoblast infiltration into a tumour spheroid. In one embodiment, the tumour spheroid comprises cells selected from one or more of HT-29 cells, HS-5 cells, MS-5 cells and MEF cells, e.g. HT-29 cells. In a one embodiment, the antibody or antigen-binding fragment induces a percentage of maximum infiltration of T-cell lymphoblast (e.g. CCRF-HSB-2 cells) into a tumour spheroid of from 40 to 100%, from 50 to 100%, from 60 to 100%, from 70 to 100%, from 80 to 100%, from 90 to 100%, from 95 to 100% or from 97 to 100%. In a one embodiment, the percentage of maximum infiltration from 10 to 100%, from 20 to 90%, from 30 to 90%, from 40 to 90%, from 50 to 90%, from 60 to 90%, from 70 to 90%, from 80 to 90%, from 90 to 90%, from 95 to 90% or from 97 to 90%, in particular from 50 to 100%. In one embodiment, T-cell lymphoblast infiltration into a spheroid is measured using live-cell analysis (e.g. IncuCyte™ S3 Live Cell Analysis System, as described elsewhere herein). In one embodiment, modulation of T-cell infiltration into cancer spheroids is carried out essentially as described in Example 15 herein.

In one embodiment, the anti-CXCR4 antibodies or antigen-binding fragments described herein (significantly) increase cell mobilisation as compared to a buffer (e.g. phosphate buffered saline (PBS)) control, such as CD45⁺ cell (leukocyte) mobilisation. In one embodiment, cell mobilisation is measured by a cell counter or flow cytometry, in particular flow cytometry. In one embodiment, the increase in cell mobilisation is measured as the mean increase in CD45⁺ cell compared to a control (e.g. a vehicle control such as PBS or an isotype control), optionally wherein the antibody or antigen-binding fragment significantly increases mean increase CD45⁺ cell mobilisation. In one embodiment, CD45⁺ cell mobilisation is carried out essentially as described in Examples 16-18 herein.

In one embodiment, the antibody or antigen-binding fragment comprises a constant region (e.g. C_{H} and/or C_{L}), for example as described elsewhere herein. In one embodiment the constant region is a mammalian (e.g. human, cynomolgus, rhesus, horse, dog, cat, mouse, rat etc) constant region (e.g. C_{H} and/or C_{L}), in particular a rodent constant region, for example murine constant region, such as a mouse or rat constant region. In a preferred embodiment, the antibody or antigen-binding fragment comprises a human constant region (e.g. C_{H} and/or C_{L}).

In one embodiment, the heavy chain constant region can be altered, e.g., mutated, to modify the properties of the antibody, e.g. to increase or decrease one or more of: Fc receptor binding, antibody glycosylation, effector cell function, and/or complement function. See, e.g., EP2654790, U.S. Publication No. 2012/0251531; U.S. Pat. Nos. 9,133,274, and 10,239,944; and PCT Publication Nos. WO2014/065945, WO2015/150447, and WO2021/016571, each of which is incorporated herein by reference their entirety.

In some embodiments, the heavy chain constant region lacks Fc effector function i.e., is an effector-null constant region. In one embodiment, the heavy chain constant region comprises mutations that decrease binding to Fc-γ receptors and/or C1q as compared to wild-type. In one embodiment, the heavy chain constant region may lack measurable binding to human FcγRI, FcγRIIa, FcγRIIa and FcγRIIIb receptors but maintain binding to human FcγRIIIb receptor and optionally maintain binding to human FcRn receptor. FcγRI, FcγRIIa, FcγRIIIa and FcγRIIb are examples of activating receptors. FcγRIIb is an example of an inhibitory receptor. FcRn is an example of a recycling receptor.

In certain embodiments, the antibody or antigen-binding fragment comprises a human IgG4 constant region, for example comprising the amino acid sequences of SEQ ID NOs: 258 to 263. In one embodiment, the IgG4 heavy chain constant region comprises mutations that reduce binding to Fc-γ receptors and/or C1q as compared to wild-type. For example, the constant region may comprise a Leu235Glu (using EU index) mutation. Another optional mutation for the IgG4 heavy chain constant region is a Ser228Pro (using EU index) mutation, which increases stability. In one embodiment, the human IgG4 constant region may comprise both Leu235Glu and Ser228Pro (using EU index) mutations. This “IgG4-PE” heavy chain constant region is effector null. In a preferred embodiment, the constant region is an IgG4-PE constant region (optionally selected from SEQ ID NO: 267 or 305). In a preferred embodiment, the IgG4-PE constant region is encoded by any of the nucleic acid sequences of SEQ ID NOs: 264 to 266 or 304.

In certain embodiments, the antibody or antigen-binding fragment comprises a human IgG1 constant region, which comprises a mutation that decreases effector function (i.e. a disabled IgG1 constant region). In one embodiment, the IgG1 constant region comprises mutations that reduce binding to Fc-γ receptors and/or C1q as compared to wild-type, such as substitutions at one or more of the amino acids selected from the group consisting of Leu234, Leu235, Gly237, Asp265, Asp270, Asn297, Ala327, Pro329, and Pro331 (using EU Index). In one embodiment, the IgG1 constant region comprises Leu235Ala and/or Gly237Ala (using EU index) mutations (e.g. amino acid sequence SEQ ID NO: 249, optionally encoded by nucleotide sequence SEQ ID NO: 248). In another embodiment, the IgG1 constant region comprises Leu234Phe, Leu235Glu and Pro331Ser (using EU Index) mutations (e.g. amino acid sequence SEQ ID NO: 307, optionally encoded by nucleotide sequence SEQ ID NO: 306).

Any of the antibodies or antigen-binding fragments of the invention may comprise a constant region, such as a rodent constant region or a human constant region (e.g. an IgG4 constant region or an IgG1 constant region), for example an effector-null human constant region (e.g. IgG4), optionally wherein the constant region is IgG4-PE (SEQ ID NO: 267 or SEQ ID NO: 305) or a disabled IgG1 (e.g. SEQ ID NO: 249 or SEQ ID NO: 307).

Constant regions, such as IgG4 or IgG1 constant regions as described elsewhere herein, often terminate with a C-terminal lysine, which is sometimes clipped, for example during protein production or downstream processing. It is intended that any antibody sequence described herein which comprises a C-terminal lysine also encompasses embodiments where such a lysine is post-translationally deleted (i.e. clipped). Thus, in one embodiment, the constant region is IgG4 (e.g. fully human IgG4, or mutant IgG4) and the IgG4 constant region comprises a C-terminal lysine or does not comprise a C-terminal lysine (lysine-clipped). In a preferred embodiment, the constant region is IgG4-PE and comprises a C-terminus lysine (e.g. SEQ ID NO: 267) or does not contain a C-terminal lysine (lysine-clipped) (e.g. SEQ ID NO: 305).

In another embodiment, the constant region is wild-type human IgG1 (optionally selected from SEQ ID NOs: 241, 243, 245 and 247). For example, the constant region is an effector-enabled IgG1 constant region, optionally having ADCC and/or CDC activity. In one embodiment, the constant region is engineered for enhanced ADCC and/or CDC and/or ADCP. The antibody-dependent cell phagocytosis (ADCP) mechanism is discussed in Gül et al., “Antibody-Dependent Phagocytosis of Tumour Cells by Macrophages: A Potent Effector Mechanism of Monoclonal Antibody Therapy of Cancer”, Cancer Res., 75(23), Dec. 1, 2015.

Without being bound by theory, an antibody or antigen-binding fragment of the invention which comprises a heavy chain constant region comprising Leu235Glu and/or Ser228Pro (using EU index) mutations (e.g. an IgG4-PE constant region, optionally SEQ ID NO: 267 or 305), may provide an improved yield of antibody or antigen-binding fragment during production or may enhance the biological properties of the antibody or antigen-binding fragment, for example in any of the assays described herein. For example, these mutations may reduce the level of apoptosis induced by the antibody or antigen-binding fragment (for example by direct effect on CXCR4 or through the effect of inhibiting one or more of its native ligands) in comparison to the same antibody or antigen-binding fragment variable region(s) comprising a different heavy chain constant region (e.g. IgG1, disabled IgG1, an IgG1 mutant or IgG4). The constant region sequence may contribute to the internalisation profile of the antibody or antigen-binding fragments or may result in the antibody or antigen-binding fragments having a shorter half-life than the same antibody or antigen-binding fragment variable region(s) comprising a different constant region (e.g. IgG1, IgG4, disabled IgG1 or an IgG1 mutant).

In one embodiment, the antibody or antigen-binding fragment comprises a light chain, such as a kappa light chain. In one embodiment, the antibody or antigen-binding fragment comprises a light chain constant region, such as a kappa light chain constant region. Kappa light chain constant region amino acid sequences and nucleotide sequences are as described in SEQ ID NOs: 271 to 279. In another embodiment, the antibody or antigen-binding fragment comprises a lambda light chain, and/or a lambda light chain constant region. Lambda light chain constant region amino acid sequences and nucleotide sequences are described in SEQ ID NOs: 280 to 303.

In one embodiment, the antibody comprises two full length heavy chains and two full length light chains (each comprising V_{H} and C_{H} and V_{L} and C_{L}).

In one embodiment, the antibody or antigen-binding fragment is a multispecific (e.g. bispecific) antibody or fusion protein, for example a bispecific antibody as described hereinabove.

In one embodiment, the antibody or antigen-binding fragment thereof comprises a V_{H} domain comprising a CDRH3, wherein
a) the CDRH3 amino acid sequence is selected from SEQ ID NOs: 12, 15, 32, 35, 52, 55, 72, 75, 92 and 95 each of which comprises 10, 9, 8, 7, 6, 5, 4, 3, 2 or 1 amino acid substitution(s);
b) the CDRH3 amino acid sequence is identical to a sequence selected from SEQ ID NOs: 12, 15, 32, 35, 52, 55, 72, 75, 92 and 95;
c) the CDRH3 amino acid sequence is as defined by IMGT or Kabat and is from a V_{H} domain selected from SEQ ID NOs: 112, 120, 128, 136, 144, 152, 160, 168, 176, 184, 192, 200, 208, 216, 224 and 232 wherein the CDRH3 sequence each comprises 10, 9, 8, 7, 6, 5, 4, 3, 2 or 1 amino acid substitution(s); or
d) the CDRH3 amino acid sequence is identical to a CDRH3 amino acid sequence as defined by IMGT or Kabat and is from a V_{H} domain selected from SEQ ID NOs: 112, 120, 128, 136, 144, 152, 160, 168, 176, 184, 192, 200, 208, 216, 224 and 232.

In one embodiment, the antibody or antigen-binding fragment comprises a V_{H} domain, wherein
a. the V_{H} domain comprises:
   i. the CDRH3 amino acid sequence of SEQ ID NOs: 12, or 15, or SEQ ID NOs: 12 or 15 each of which comprises 10, 9, 8, 7, 6, 5, 4, 3, 2 or 1 amino acid substitution(s); and/or
   ii. a CDRH1 amino acid sequence of SEQ ID NOs: 10 or 13, or SEQ ID NOs: 10 or 13 each of which comprises 10, 9, 8, 7, 6, 5, 4, 3, 2 or 1 amino acid substitution(s); and/or
   iii. a CDRH2 amino acid sequence of SEQ ID NOs: 11 or 14, or SEQ ID NOs: 11 or 14 each of which comprises 10, 9, 8, 7, 6, 5, 4, 3, 2 or 1 amino acid substitution(s),
b. the V_{H} domain comprises:
   i. the CDRH3 amino acid sequence of SEQ ID NOs: 32 or 35, or SEQ ID NOs: 32 or 35 each of which comprises 10, 9, 8, 7, 6, 5, 4, 3, 2 or 1 amino acid substitution(s); and/or
   ii. a CDRH1 amino acid sequence of SEQ ID NOs: 30 or 33, or SEQ ID NOs: 30 or 33 each of which comprises 10, 9, 8, 7, 6, 5, 4, 3, 2 or 1 amino acid substitution(s); and/or
   iii. a CDRH2 amino acid sequence of SEQ ID NOs: 31 or 34, or SEQ ID NOs: 31 or 34 each of which comprises 10, 9, 8, 7, 6, 5, 4, 3, 2 or 1 amino acid substitution(s),
c. the V_{H} domain comprises:
   i. the CDRH3 amino acid sequence of SEQ ID NOs: 52 or 55, or SEQ ID NOs: 52 or 55 each of which comprises 10, 9, 8, 7, 6, 5, 4, 3, 2 or 1 amino acid substitution(s); and/or
   ii. a CDRH1 amino acid sequence of SEQ ID NOs: 50 or 53, or SEQ ID NOs: 50 or 53 each of which comprises 10, 9, 8, 7, 6, 5, 4, 3, 2 or 1 amino acid substitution(s); and/or
   iii. a CDRH2 amino acid sequence of SEQ ID NOs: 51 or 54, or SEQ ID NOs: 51 or 54 each of which comprises 10, 9, 8, 7, 6, 5, 4, 3, 2 or 1 amino acid substitution(s),
d. the V_{H} domain comprises:
   i. the CDRH3 amino acid sequence of SEQ ID NOs: 72 or 75, or SEQ ID NOs: 72 or 75 each of which comprises 10, 9, 8, 7, 6, 5, 4, 3, 2 or 1 amino acid substitution(s); and/or
   ii. a CDRH1 amino acid sequence of SEQ ID NOs: 70 or 73, or SEQ ID NOs: 70 or 73 each of which comprises 10, 9, 8, 7, 6, 5, 4, 3, 2 or 1 amino acid substitution(s); and/or
   iii. a CDRH2 amino acid sequence of SEQ ID NOs: 71 or 74, or SEQ ID NOs: 71 or 74 each of which comprises 10, 9, 8, 7, 6, 5, 4, 3, 2 or 1 amino acid substitution(s),
e. the V_{H} domain comprises:
   i. the CDRH3 amino acid sequence of SEQ ID NOs: 92 or 95, or SEQ ID NOs: 92 or 95 each of which comprises 10, 9, 8, 7, 6, 5, 4, 3, 2 or 1 amino acid substitution(s); and/or
   ii. a CDRH1 amino acid sequence of SEQ ID NOs: 90 or 93, or SEQ ID NOs: 90 or 93 each of which comprises 10, 9, 8, 7, 6, 5, 4, 3, 2 or 1 amino acid substitution(s); and/or
   iii. a CDRH2 amino acid sequence of SEQ ID NOs: 91 or 94, or SEQ ID NOs: 91 or 94 each of which comprises 10, 9, 8, 7, 6, 5, 4, 3, 2 or 1 amino acid substitution(s),
f. the V_{H} domain comprises:
   i. the CDRH3 amino acid sequence is as defined by IMGT or Kabat and is from a V_{H} domain selected from SEQ ID NO: 112, or wherein the CDRH3 amino acid sequence is as defined by IMGT or Kabat and is from a V_{H} domain selected from SEQ ID NO: 112 and comprises 10, 9, 8, 7, 6, 5, 4, 3, 2 or 1 amino acid substitution(s); and/or
   ii. a CDRH1 amino acid sequence is as defined by IMGT or Kabat and is from a V_{H} domain selected from SEQ ID NO: 112, or wherein the CDRH1 amino acid sequence is as defined by IMGT or Kabat and is from a V_{H} domain selected from SEQ ID NO: 112 and comprises 10, 9, 8, 7, 6, 5, 4, 3, 2 or 1 amino acid substitution(s); and/or
   iii. a CDRH2 amino acid sequence is as defined by IMGT or Kabat and is from a V_{H} domain selected from SEQ ID NO: 112, or wherein the CDRH2 amino acid sequence is as defined by IMGT or Kabat and is from a V_{H} domain selected from SEQ ID NO: 112 and comprises 10, 9, 8, 7, 6, 5, 4, 3, 2 or 1 amino acid substitution(s),
g. the V_{H} domain comprises:
   i. the CDRH3 amino acid sequence is as defined by IMGT or Kabat and is from a V_{H} domain selected from SEQ ID NO: 120, or wherein the CDRH3 amino acid sequence is as defined by IMGT or Kabat and is from a V_{H} domain selected from SEQ ID NO: 120 and comprises 10, 9, 8, 7, 6, 5, 4, 3, 2 or 1 amino acid substitution(s); and/or
   ii. a CDRH1 amino acid sequence is as defined by IMGT or Kabat and is from a V_{H} domain selected from SEQ ID NO: 120, or wherein the CDRH1 amino acid sequence is as defined by IMGT or Kabat and is from a V_{H} domain selected from SEQ ID NO: 120 and comprises 10, 9, 8, 7, 6, 5, 4, 3, 2 or 1 amino acid substitution(s); and/or
   iii. a CDRH2 amino acid sequence is as defined by IMGT or Kabat and is from a V_{H} domain selected from SEQ ID NO: 120, or wherein the CDRH2 amino acid sequence is as defined by IMGT or Kabat and is from a V_{H} domain selected from SEQ ID NO: 120 and comprises 10, 9, 8, 7, 6, 5, 4, 3, 2 or 1 amino acid substitution(s),
h. the V_{H} domain comprises:
   i. the CDRH3 amino acid sequence is as defined by IMGT or Kabat and is from a V_{H} domain selected from SEQ ID NO: 128, or wherein the CDRH3 amino acid sequence is as defined by IMGT or Kabat and is from a V_{H} domain selected from SEQ ID NO: 128 and comprises 10, 9, 8, 7, 6, 5, 4, 3, 2 or 1 amino acid substitution(s); and/or
   ii. a CDRH1 amino acid sequence is as defined by IMGT or Kabat and is from a V_{H} domain selected from SEQ ID NO: 128, or wherein the CDRH1 amino acid sequence is as defined by IMGT or Kabat and is from a V_{H} domain selected from SEQ ID NO: 128 and comprises 10, 9, 8, 7, 6, 5, 4, 3, 2 or 1 amino acid substitution(s); and/or
   iii. a CDRH2 amino acid sequence is as defined by IMGT or Kabat and is from a V_{H} domain selected from SEQ ID NO: 128, or wherein the CDRH2 amino acid sequence is as defined by IMGT or Kabat and is from a V_{H} domain selected from SEQ ID NO: 128 and comprises 10, 9, 8, 7, 6, 5, 4, 3, 2 or 1 amino acid substitution(s),
i. the V_{H} domain comprises:
   i. the CDRH3 amino acid sequence is as defined by IMGT or Kabat and is from a V_{H} domain selected from SEQ ID NO: 136, or wherein the CDRH3 amino acid sequence is as defined by IMGT or Kabat and is from a V_{H} domain selected from SEQ ID NO: 136 and comprises 10, 9, 8, 7, 6, 5, 4, 3, 2 or 1 amino acid substitution(s); and/or
   ii. a CDRH1 amino acid sequence is as defined by IMGT or Kabat and is from a V_{H} domain selected from SEQ ID NO: 136, or wherein the CDRH1 amino acid sequence is as defined by IMGT or Kabat and is from a V_{H} domain selected from SEQ ID NO: 136 and comprises 10, 9, 8, 7, 6, 5, 4, 3, 2 or 1 amino acid substitution(s); and/or
   iii. a CDRH2 amino acid sequence is as defined by IMGT or Kabat and is from a V_{H} domain selected from SEQ ID NO: 136, or wherein the CDRH2 amino acid sequence is as defined by IMGT or Kabat and is from a V_{H} domain selected from SEQ ID NO: 136 and comprises 10, 9, 8, 7, 6, 5, 4, 3, 2 or 1 amino acid substitution(s),
j. the V_{H} domain comprises:
   i. the CDRH3 amino acid sequence is as defined by IMGT or Kabat and is from a V_{H} domain selected from SEQ ID NO: 144, or wherein the CDRH3 amino acid sequence is as defined by IMGT or Kabat and is from a V_{H} domain selected from SEQ ID NO: 144 and comprises 10, 9, 8, 7, 6, 5, 4, 3, 2 or 1 amino acid substitution(s); and/or
   ii. a CDRH1 amino acid sequence is as defined by IMGT or Kabat and is from a V_{H} domain selected from SEQ ID NO: 144, or wherein the CDRH1 amino acid sequence is as defined by IMGT or Kabat and is from a V_{H} domain selected from SEQ ID NO: 144 and comprises 10, 9, 8, 7, 6, 5, 4, 3, 2 or 1 amino acid substitution(s); and/or
   iii. a CDRH2 amino acid sequence is as defined by IMGT or Kabat and is from a V_{H} domain selected from SEQ ID NO: 144, or wherein the CDRH2 amino acid sequence is as defined by IMGT or Kabat and is from a V_{H} domain selected from SEQ ID NO: 144 and comprises 10, 9, 8, 7, 6, 5, 4, 3, 2 or 1 amino acid substitution(s),
k. the V_{H} domain comprises:
   i. the CDRH3 amino acid sequence is as defined by IMGT or Kabat and is from a V_{H} domain selected from SEQ ID NO: 152, or wherein the CDRH3 amino acid sequence is as defined by IMGT or Kabat and is from a V_{H} domain selected from SEQ ID NO: 152 and comprises 10, 9, 8, 7, 6, 5, 4, 3, 2 or 1 amino acid substitution(s); and/or
   ii. a CDRH1 amino acid sequence is as defined by IMGT or Kabat and is from a V_{H} domain selected from SEQ ID NO: 152, or wherein the CDRH1 amino acid sequence is as defined by IMGT or Kabat and is from a V_{H} domain selected from SEQ ID NO: 152 and comprises 10, 9, 8, 7, 6, 5, 4, 3, 2 or 1 amino acid substitution(s); and/or
   iii. a CDRH2 amino acid sequence is as defined by IMGT or Kabat and is from a V_{H} domain selected from SEQ ID NO: 152, or wherein the CDRH2 amino acid sequence is as defined by IMGT or Kabat and is from a V_{H} domain selected from SEQ ID NO: 152 and comprises 10, 9, 8, 7, 6, 5, 4, 3, 2 or 1 amino acid substitution(s),
l. the V_{H} domain comprises:
   i. the CDRH3 amino acid sequence is as defined by IMGT or Kabat and is from a V_{H} domain selected from SEQ ID NO: 160, or wherein the CDRH3 amino acid sequence is as defined by IMGT or Kabat and is from a V_{H} domain selected from SEQ ID NO: 160 and comprises 10, 9, 8, 7, 6, 5, 4, 3, 2 or 1 amino acid substitution(s); and/or
   ii. a CDRH1 amino acid sequence is as defined by IMGT or Kabat and is from a V_{H} domain selected from SEQ ID NO: 160, or wherein the CDRH1 amino acid sequence is as defined by IMGT or Kabat and is from a V_{H} domain selected from SEQ ID NO: 160 and comprises 10, 9, 8, 7, 6, 5, 4, 3, 2 or 1 amino acid substitution(s); and/or
   iii. a CDRH2 amino acid sequence is as defined by IMGT or Kabat and is from a V_{H} domain selected from SEQ ID NO: 160, or wherein the CDRH2 amino acid sequence is as defined by IMGT or Kabat and is from a V_{H} domain selected from SEQ ID NO: 160 and comprises 10, 9, 8, 7, 6, 5, 4, 3, 2 or 1 amino acid substitution(s),
m. the V_{H} domain comprises:
   i. the CDRH3 amino acid sequence is as defined by IMGT or Kabat and is from a V_{H} domain selected from SEQ ID NO: 168, or wherein the CDRH3 amino acid sequence is as defined by IMGT or Kabat and is from a V_{H} domain selected from SEQ ID NO: 168 and comprises 10, 9, 8, 7, 6, 5, 4, 3, 2 or 1 amino acid substitution(s); and/or
   ii. a CDRH1 amino acid sequence is as defined by IMGT or Kabat and is from a V_{H} domain selected from SEQ ID NO: 168, or wherein the CDRH1 amino acid sequence is as defined by IMGT or Kabat and is from a V_{H} domain selected from SEQ ID NO: 168 and comprises 10, 9, 8, 7, 6, 5, 4, 3, 2 or 1 amino acid substitution(s); and/or
   iii. a CDRH2 amino acid sequence is as defined by IMGT or Kabat and is from a V_{H} domain selected from SEQ ID NO: 168, or wherein the CDRH2 amino acid sequence is as defined by IMGT or Kabat and is from a V_{H} domain selected from SEQ ID NO: 168 and comprises 10, 9, 8, 7, 6, 5, 4, 3, 2 or 1 amino acid substitution(s),
n. the V_{H} domain comprises:
   i. the CDRH3 amino acid sequence is as defined by IMGT or Kabat and is from a V_{H} domain selected from SEQ ID NO: 176, or wherein the CDRH3 amino acid sequence is as defined by IMGT or Kabat and is from a V_{H} domain selected from SEQ ID NO: 176 and comprises 10, 9, 8, 7, 6, 5, 4, 3, 2 or 1 amino acid substitution(s); and/or
   ii. a CDRH1 amino acid sequence is as defined by IMGT or Kabat and is from a V_{H} domain selected from SEQ ID NO: 176, or wherein the CDRH1 amino acid sequence is as defined by IMGT or Kabat and is from a V_{H} domain selected from SEQ ID NO: 176 and comprises 10, 9, 8, 7, 6, 5, 4, 3, 2 or 1 amino acid substitution(s); and/or
   iii. a CDRH2 amino acid sequence is as defined by IMGT or Kabat and is from a V_{H} domain selected from SEQ ID NO: 176, or wherein the CDRH2 amino acid sequence is as defined by IMGT or Kabat and is from a V_{H} domain selected from SEQ ID NO: 176 and comprises 10, 9, 8, 7, 6, 5, 4, 3, 2 or 1 amino acid substitution(s),
o. the V_{H} domain comprises:
   i. the CDRH3 amino acid sequence is as defined by IMGT or Kabat and is from a V_{H} domain selected from SEQ ID NO: 184, or wherein the CDRH3 amino acid sequence is as defined by IMGT or Kabat and is from a V_{H} domain selected from SEQ ID NO: 184 and comprises 10, 9, 8, 7, 6, 5, 4, 3, 2 or 1 amino acid substitution(s); and/or
   ii. a CDRH1 amino acid sequence is as defined by IMGT or Kabat and is from a V_{H} domain selected from SEQ ID NO: 184, or wherein the CDRH1 amino acid sequence is as defined by IMGT or Kabat and is from a V_{H} domain selected from SEQ ID NO: 184 and comprises 10, 9, 8, 7, 6, 5, 4, 3, 2 or 1 amino acid substitution(s); and/or
   iii. a CDRH2 amino acid sequence is as defined by IMGT or Kabat and is from a V_{H} domain selected from SEQ ID NO: 184, or wherein the CDRH2 amino acid sequence is as defined by IMGT or Kabat and is from a V_{H} domain selected from SEQ ID NO: 184 and comprises 10, 9, 8, 7, 6, 5, 4, 3, 2 or 1 amino acid substitution(s),
p. the V_{H} domain comprises:
   i. the CDRH3 amino acid sequence is as defined by IMGT or Kabat and is from a V_{H} domain selected from SEQ ID NO: 192, or wherein the CDRH3 amino acid sequence is as defined by IMGT or Kabat and is from a V_{H} domain selected from SEQ ID NO: 192 and comprises 10, 9, 8, 7, 6, 5, 4, 3, 2 or 1 amino acid substitution(s); and/or
   ii. a CDRH1 amino acid sequence is as defined by IMGT or Kabat and is from a V_{H} domain selected from SEQ ID NO: 192, or wherein the CDRH1 amino acid sequence is as defined by IMGT or Kabat and is from a V_{H} domain selected from SEQ ID NO: 192 and comprises 10, 9, 8, 7, 6, 5, 4, 3, 2 or 1 amino acid substitution(s); and/or
   iii. a CDRH2 amino acid sequence is as defined by IMGT or Kabat and is from a V_{H} domain selected from SEQ ID NO: 192, or wherein the CDRH2 amino acid sequence is as defined by IMGT or Kabat and is from a V_{H} domain selected from SEQ ID NO: 192 and comprises 10, 9, 8, 7, 6, 5, 4, 3, 2 or 1 amino acid substitution(s),
q. the V_{H} domain comprises:
   i. the CDRH3 amino acid sequence is as defined by IMGT or Kabat and is from a V_{H} domain selected from SEQ ID NO: 200, or wherein the CDRH3 amino acid sequence is as defined by IMGT or Kabat and is from a V_{H} domain selected from SEQ ID NO: 200 and comprises 10, 9, 8, 7, 6, 5, 4, 3, 2 or 1 amino acid substitution(s); and/or
   ii. a CDRH1 amino acid sequence is as defined by IMGT or Kabat and is from a V_{H} domain selected from SEQ ID NO: 200, or wherein the CDRH1 amino acid sequence is as defined by IMGT or Kabat and is from a V_{H} domain selected from SEQ ID NO: 200 and comprises 10, 9, 8, 7, 6, 5, 4, 3, 2 or 1 amino acid substitution(s); and/or
   iii. a CDRH2 amino acid sequence is as defined by IMGT or Kabat and is from a V_{H} domain selected from SEQ ID NO: 200, or wherein the CDRH2 amino acid sequence is as defined by IMGT or Kabat and is from a V_{H} domain selected from SEQ ID NO: 200 and comprises 10, 9, 8, 7, 6, 5, 4, 3, 2 or 1 amino acid substitution(s),
r. the V_{H} domain comprises:
   i. the CDRH3 amino acid sequence is as defined by IMGT or Kabat and is from a V_{H} domain selected from SEQ ID NO: 208, or wherein the CDRH3 amino acid sequence is as defined by IMGT or Kabat and is from a V_{H} domain selected from SEQ ID NO: 208 and comprises 10, 9, 8, 7, 6, 5, 4, 3, 2 or 1 amino acid substitution(s); and/or
   ii. a CDRH1 amino acid sequence is as defined by IMGT or Kabat and is from a V_{H} domain selected from SEQ ID NO: 208, or wherein the CDRH1 amino acid sequence is as defined by IMGT or Kabat and is from a V_{H} domain selected from SEQ ID NO: 208 and comprises 10, 9, 8, 7, 6, 5, 4, 3, 2 or 1 amino acid substitution(s); and/or
   iii. a CDRH2 amino acid sequence is as defined by IMGT or Kabat and is from a V_{H} domain selected from SEQ ID NO: 208, or wherein the CDRH2 amino acid sequence is as defined by IMGT or Kabat and is from a V_{H} domain selected from SEQ ID NO: 208 and comprises 10, 9, 8, 7, 6, 5, 4, 3, 2 or 1 amino acid substitution(s),
s. the V_{H} domain comprises:
   i. the CDRH3 amino acid sequence is as defined by IMGT or Kabat and is from a V_{H} domain selected from SEQ ID NO: 216, or wherein the CDRH3 amino acid sequence is as defined by IMGT or Kabat and is from a V_{H} domain selected from SEQ ID NO: 216 and comprises 10, 9, 8, 7, 6, 5, 4, 3, 2 or 1 amino acid substitution(s); and/or
   ii. a CDRH1 amino acid sequence is as defined by IMGT or Kabat and is from a V_{H} domain selected from SEQ ID NO: 216, or wherein the CDRH1 amino acid sequence is as defined by IMGT or Kabat and is from a V_{H} domain selected from SEQ ID NO: 216 and comprises 10, 9, 8, 7, 6, 5, 4, 3, 2 or 1 amino acid substitution(s); and/or
   iii. a CDRH2 amino acid sequence is as defined by IMGT or Kabat and is from a V_{H} domain selected from SEQ ID NO: 216, or wherein the CDRH2 amino acid sequence is as defined by IMGT or Kabat and is from a V_{H} domain selected from SEQ ID NO: 216 and comprises 10, 9, 8, 7, 6, 5, 4, 3, 2 or 1 amino acid substitution(s),
t. the V_{H} domain comprises:
   i. the CDRH3 amino acid sequence is as defined by IMGT or Kabat and is from a V_{H} domain selected from SEQ ID NO: 224, or wherein the CDRH3 amino acid sequence is as defined by IMGT or Kabat and is from a V_{H} domain selected from SEQ ID NO: 224 and comprises 10, 9, 8, 7, 6, 5, 4, 3, 2 or 1 amino acid substitution(s); and/or
   ii. a CDRH1 amino acid sequence is as defined by IMGT or Kabat and is from a V_{H} domain selected from SEQ ID NO: 224, or wherein the CDRH1 amino acid sequence is as defined by IMGT or Kabat and is from a V_{H} domain selected from SEQ ID NO: 224 comprising 10, 9, 8, 7, 6, 5, 4, 3, 2 or 1 amino acid substitution(s); and/or
   iii. a CDRH2 amino acid sequence is as defined by IMGT or Kabat and is from a V_{H} domain selected from SEQ ID NO: 224, wherein the CDRH2 amino acid sequence is as defined by IMGT or Kabat and is from a V_{H} domain selected from or SEQ ID NO: 224 comprising 10, 9, 8, 7, 6, 5, 4, 3, 2 or 1 amino acid substitution(s), or
u. the V_{H} domain comprises:
   i. the CDRH3 amino acid sequence is as defined by IMGT or Kabat and is from a V_{H} domain selected from SEQ ID NO: 232, or wherein the CDRH3 amino acid sequence is as defined by IMGT or Kabat and is from a V_{H} domain selected from SEQ ID NO: 232 comprising 10, 9, 8, 7, 6, 5, 4, 3, 2 or 1 amino acid substitution(s); and/or
   ii. a CDRH1 amino acid sequence is as defined by IMGT or Kabat and is from a V_{H} domain selected from SEQ ID NO: 232, or wherein the CDRH1 amino acid sequence is as defined by IMGT or Kabat and is from a V_{H} domain selected from SEQ ID NO: 232 comprising 10, 9, 8, 7, 6, 5, 4, 3, 2 or 1 amino acid substitution(s); and/or
   iii. a CDRH2 amino acid sequence is as defined by IMGT or Kabat and is from a V_{H} domain selected from SEQ ID NO: 232, or wherein the CDRH2 amino acid sequence is as defined by IMGT or Kabat and is from a V_{H} domain selected from SEQ ID NO: 232 comprising 10, 9, 8, 7, 6, 5, 4, 3, 2 or 1 amino acid substitution(s).

In one embodiment, the antibody or antigen-binding fragment comprises, or further comprises, a V_{L} domain comprising a CDRL3, wherein
a. the CDRL3 amino acid sequence is selected from SEQ ID NOs: 22, 25, 42, 45, 62, 65, 82, 85, 102 and 105 each of which comprises 10, 9, 8, 7, 6, 5, 4, 3, 2 or 1 amino acid substitution(s);
b. the CDRL3 amino acid sequence is identical to a sequence selected from SEQ ID NOs: 22, 25, 42, 45, 62, 65, 82, 85, 102 and 105;
c. the CDRL3 amino acid sequence is as defined by IMGT or Kabat and is from a V_{L} domain selected from SEQ ID NOs: 116, 124, 132, 140, 148, 156, 164, 172, 180, 188, 196, 204, 212, 220, 228 and 236 wherein the CDRL3 sequence each comprises 10, 9, 8, 7, 6, 5, 4, 3, 2 or 1 amino acid substitution(s); or
d. the CDRL3 amino acid sequence is identical to a CDRL3 amino acid sequence as defined by IMGT or Kabat and is from a V_{L} domain selected from SEQ ID NOs: 116, 124, 132, 140, 148, 156, 164, 172, 180, 188, 196, 204, 212, 220, 228 and 236.

In one embodiment, the antibody or antigen-binding fragment comprises, or further comprises, a V_{L} domain wherein
a. the V_{L} domain comprises:
   i. the CDRL3 amino acid sequence of SEQ ID NOs: 22 or 25 or SEQ ID NOs: 22 or 25 each of which comprises 10, 9, 8, 7, 6, 5, 4, 3, 2 or 1 amino acid substitution(s); and/or
   ii. a CDRL1 amino acid sequence of SEQ ID NOs: 20 or 23, or SEQ ID NOs: 20 or 23 each of which comprises 10, 9, 8, 7, 6, 5, 4, 3, 2 or 1 amino acid substitution(s); and/or
   iii. a CDRL2 amino acid sequence of SEQ ID NOs: 21 or 24, or SEQ ID NOs: 21 or 24 each of which comprises 10, 9, 8, 7, 6, 5, 4, 3, 2 or 1 amino acid substitution(s),
b. the V_{L} domain comprises:
   i. the CDRL3 amino acid sequence of SEQ ID NOs: 42 or 45 or SEQ ID NOs: 42 or 45 each of which comprises 10, 9, 8, 7, 6, 5, 4, 3, 2 or 1 amino acid substitution(s); and/or
   ii. a CDRL1 amino acid sequence of SEQ ID NOs: 40 or 43, or SEQ ID NOs: 40 or 43 each of which comprises 10, 9, 8, 7, 6, 5, 4, 3, 2 or 1 amino acid substitution(s); and/or
   iii. a CDRL2 amino acid sequence of SEQ ID NOs: 41 or 44, or SEQ ID NOs: 41 or 44 each of which comprises 10, 9, 8, 7, 6, 5, 4, 3, 2 or 1 amino acid substitution(s),
c. the V_{L} domain comprises:
   i. the CDRL3 amino acid sequence of SEQ ID NOs: 62 or 65 or SEQ ID NOs: 62 or 65 each of which comprises 10, 9, 8, 7, 6, 5, 4, 3, 2 or 1 amino acid substitution(s); and/or
   ii. a CDRL1 amino acid sequence of SEQ ID NOs: 60 or 63, or SEQ ID NOs: 60 or 63 each of which comprises 10, 9, 8, 7, 6, 5, 4, 3, 2 or 1 amino acid substitution(s); and/or
   iii. a CDRL2 amino acid sequence of SEQ ID NOs: 61 or 64, or SEQ ID NOs: 61 or 64 each of which comprises 10, 9, 8, 7, 6, 5, 4, 3, 2 or 1 amino acid substitution(s),
d. the V_{L} domain comprises:
   i. the CDRL3 amino acid sequence of SEQ ID NOs: 82 or 85 or SEQ ID NOs: 82 or 85 each of which comprises 10, 9, 8, 7, 6, 5, 4, 3, 2 or 1 amino acid substitution(s); and/or
   ii. a CDRL1 amino acid sequence of SEQ ID NOs: 80 or 83, or SEQ ID NOs: 80 or 83 each of which comprises 10, 9, 8, 7, 6, 5, 4, 3, 2 or 1 amino acid substitution(s); and/or
   iii. a CDRL2 amino acid sequence of SEQ ID NOs: 81 or 84, or SEQ ID NOs: 81 or 84 each of which comprises 10, 9, 8, 7, 6, 5, 4, 3, 2 or 1 amino acid substitution(s),
e. the V_{L} domain comprises:
   i. the CDRL3 amino acid sequence of SEQ ID NOs: 102 or 105 or SEQ ID NOs: 102 or 105 each of which comprises 10, 9, 8, 7, 6, 5, 4, 3, 2 or 1 amino acid substitution(s); and/or
   ii. a CDRL1 amino acid sequence of SEQ ID NOs: 100 or 103, or SEQ ID NOs: 100 or 103 each of which comprises 10, 9, 8, 7, 6, 5, 4, 3, 2 or 1 amino acid substitution(s); and/or
   iii. a CDRL2 amino acid sequence of SEQ ID NOs: 101 or 104, or SEQ ID NOs: 101 or 104 each of which comprises 10, 9, 8, 7, 6, 5, 4, 3, 2 or 1 amino acid substitution(s),
f. the V_{L} domain comprises:
   i. the CDRL3 amino acid sequence is as defined by IMGT or Kabat and is from a V_{L} domain selected from SEQ ID NO: 116, or wherein the CDRL3 amino acid sequence is as defined by IMGT or Kabat and is from a V_{L} domain selected from SEQ ID NO: 116 comprising 10, 9, 8, 7, 6, 5, 4, 3, 2 or 1 amino acid substitution(s); and/or
   ii. a CDRL1 amino acid sequence is as defined by IMGT or Kabat and is from a V_{L} domain selected from SEQ ID NO: 116, or wherein the CDRL1 amino acid sequence is as defined by IMGT or Kabat and is from a V_{L} domain selected from SEQ ID NO: 116 comprising 10, 9, 8, 7, 6, 5, 4, 3, 2 or 1 amino acid substitution(s); and/or
   iii. a CDRL2 amino acid sequence is as defined by IMGT or Kabat and is from a V_{L} domain selected from SEQ ID NO: 116, or wherein the CDRL2 amino acid sequence is as defined by IMGT or Kabat and is from a V_{L} domain selected from SEQ ID NO: 116 comprising 10, 9, 8, 7, 6, 5, 4, 3, 2 or 1 amino acid substitution(s),
g. the V_{L} domain comprises:
   i. the CDRL3 amino acid sequence is as defined by IMGT or Kabat and is from a V_{L} domain selected from SEQ ID NO: 124, or wherein the CDRL3 amino acid sequence is as defined by IMGT or Kabat and is from a V_{L} domain selected from SEQ ID NO: 1294 comprising 10, 9, 8, 7, 6, 5, 4, 3, 2 or 1 amino acid substitution(s); and/or
   ii. a CDRL1 amino acid sequence is as defined by IMGT or Kabat and is from a V_{L} domain selected from SEQ ID NO: 124, or wherein the CDRL1 amino acid sequence is as defined by IMGT or Kabat and is from a V_{L} domain selected from SEQ ID NO: 124 comprising 10, 9, 8, 7, 6, 5, 4, 3, 2 or 1 amino acid substitution(s); and/or
   iii. a CDRL2 amino acid sequence is as defined by IMGT or Kabat and is from a V_{L} domain selected from SEQ ID NO: 124, or wherein the CDRL2 amino acid sequence is as defined by IMGT or Kabat and is from a V_{L} domain selected from SEQ ID NO: 124 comprising 10, 9, 8, 7, 6, 5, 4, 3, 2 or 1 amino acid substitution(s),
h. the V_{L} domain comprises:
   i. the CDRL3 amino acid sequence is as defined by IMGT or Kabat and is from a V_{L} domain selected from SEQ ID NO: 132, or wherein the CDRL3 amino acid sequence is as defined by IMGT or Kabat and is from a V_{L} domain selected from SEQ ID NO: 132 comprising 10, 9, 8, 7, 6, 5, 4, 3, 2 or 1 amino acid substitution(s); and/or
   ii. a CDRL1 amino acid sequence is as defined by IMGT or Kabat and is from a V_{L} domain selected from SEQ ID NO: 132, or wherein the CDRL1 amino acid sequence is as defined by IMGT or Kabat and is from a V_{L} domain selected from SEQ ID NO: 132 comprising 10, 9, 8, 7, 6, 5, 4, 3, 2 or 1 amino acid substitution(s); and/or
   iii. a CDRL2 amino acid sequence is as defined by IMGT or Kabat and is from a V_{L} domain selected from SEQ ID NO: 132, or wherein the CDRL2 amino acid sequence is as defined by IMGT or Kabat and is from a V_{L} domain selected from SEQ ID NO: 132 comprising 10, 9, 8, 7, 6, 5, 4, 3, 2 or 1 amino acid substitution(s),
i. the V_{L} domain comprises:
   i. the CDRL3 amino acid sequence is as defined by IMGT or Kabat and is from a V_{L} domain selected from SEQ ID NO: 140, or wherein the CDRL3 amino acid sequence is as defined by IMGT or Kabat and is from a V_{L} domain selected from SEQ ID NO: 140 comprising 10, 9, 8, 7, 6, 5, 4, 3, 2 or 1 amino acid substitution(s); and/or
   ii. a CDRL1 amino acid sequence is as defined by IMGT or Kabat and is from a V_{L} domain selected from SEQ ID NO: 140, or wherein the CDRL1 amino acid sequence is as defined by IMGT or Kabat and is from a V_{L} domain selected from SEQ ID NO: 140 comprising 10, 9, 8, 7, 6, 5, 4, 3, 2 or 1 amino acid substitution(s); and/or
   iii. a CDRL2 amino acid sequence is as defined by IMGT or Kabat and is from a V_{L} domain selected from SEQ ID NO: 140, or wherein the CDRL2 amino acid sequence is as defined by IMGT or Kabat and is from a V_{L} domain selected from SEQ ID NO: 140 comprising 10, 9, 8, 7, 6, 5, 4, 3, 2 or 1 amino acid substitution(s),
j. the V_{L} domain comprises:
   i. the CDRL3 amino acid sequence is as defined by IMGT or Kabat and is from a V_{L} domain selected from SEQ ID NO: 148, or wherein the CDRL3 amino acid sequence is as defined by IMGT or Kabat and is from a V_{L} domain selected from SEQ ID NO: 148 comprising 10, 9, 8, 7, 6, 5, 4, 3, 2 or 1 amino acid substitution(s); and/or
   ii. a CDRL1 amino acid sequence is as defined by IMGT or Kabat and is from a V_{L} domain selected from SEQ ID NO: 148, or wherein the CDRL1 amino acid sequence is as defined by IMGT or Kabat and is from a V_{L} domain selected from SEQ ID NO: 148 comprising 10, 9, 8, 7, 6, 5, 4, 3, 2 or 1 amino acid substitution(s); and/or
   iii. a CDRL2 amino acid sequence is as defined by IMGT or Kabat and is from a V_{L} domain selected from SEQ ID NO: 148, or wherein the CDRL2 amino acid sequence is as defined by IMGT or Kabat and is from a V_{L} domain selected from SEQ ID NO: 148 comprising 10, 9, 8, 7, 6, 5, 4, 3, 2 or 1 amino acid substitution(s),
k. the V_{L} domain comprises:
   i. the CDRL3 amino acid sequence is as defined by IMGT or Kabat and is from a V_{L} domain selected from SEQ ID NO: 156, or wherein the CDRL3 amino acid sequence is as defined by IMGT or Kabat and is from a V_{L} domain selected from SEQ ID NO: 156 comprising 10, 9, 8, 7, 6, 5, 4, 3, 2 or 1 amino acid substitution(s); and/or
   ii. a CDRL1 amino acid sequence is as defined by IMGT or Kabat and is from a V_{L} domain selected from SEQ ID NO: 156, or wherein the CDRL1 amino acid sequence is as defined by IMGT or Kabat and is from a V_{L} domain selected from SEQ ID NO: 156 comprising 3, 2 or 1 amino acid substitution(s); and/or
   iii. a CDRL2 amino acid sequence is as defined by IMGT or Kabat and is from a V_{L} domain selected from SEQ ID NO: 156, or wherein the CDRL2 amino acid sequence is as defined by IMGT or Kabat and is from a V_{L} domain selected from SEQ ID NO: 156 comprising 10, 9, 8, 7, 6, 5, 4, 3, 2 or 1 amino acid substitution(s),
l. the V_{L} domain comprises:
   i. the CDRL3 amino acid sequence is as defined by IMGT or Kabat and is from a V_{L} domain selected from SEQ ID NO: 164, or wherein the CDRL3 amino acid sequence is as defined by IMGT or Kabat and is from a V_{L} domain selected from SEQ ID NO: 164 comprising 10, 9, 8, 7, 6, 5, 4, 3, 2 or 1 amino acid substitution(s); and/or
   ii. a CDRL1 amino acid sequence is as defined by IMGT or Kabat and is from a V_{L} domain selected from SEQ ID NO: 164, or wherein the CDRL1 amino acid sequence is as defined by IMGT or Kabat and is from a V_{L} domain selected from SEQ ID NO: 164 comprising 10, 9, 8, 7, 6, 5, 4, 3, 2 or 1 amino acid substitution(s); and/or
   iii. a CDRL2 amino acid sequence is as defined by IMGT or Kabat and is from a V_{L} domain selected from SEQ ID NO: 164, or wherein the CDRL2 amino acid sequence is as defined by IMGT or Kabat and is from a V_{L} domain selected from SEQ ID NO: 164 comprising 10, 9, 8, 7, 6, 5, 4, 3, 2 or 1 amino acid substitution(s),
m. the V_{L} domain comprises:
   i. the CDRL3 amino acid sequence is as defined by IMGT or Kabat and is from a V_{L} domain selected from SEQ ID NO: 172, or wherein the CDRL3 amino acid sequence is as defined by IMGT or Kabat and is from a V_{L} domain selected from SEQ ID NO: 172 comprising 10, 9, 8, 7, 6, 5, 4, 3, 2 or 1 amino acid substitution(s); and/or
   ii. a CDRL1 amino acid sequence is as defined by IMGT or Kabat and is from a V_{L} domain selected from SEQ ID NO: 172, or wherein the CDRL1 amino acid sequence is as defined by IMGT or Kabat and is from a V_{L} domain selected from SEQ ID NO: 172 comprising 10, 9, 8, 7, 6, 5, 4, 3, 2 or 1 amino acid substitution(s); and/or
   iii. a CDRL2 amino acid sequence is as defined by IMGT or Kabat and is from a V_{L} domain selected from SEQ ID NO: 172, or wherein the CDRL2 amino acid sequence is as defined by IMGT or Kabat and is from a V_{L} domain selected from SEQ ID NO: 172 comprising 10, 9, 8, 7, 6, 5, 4, 3, 2 or 1 amino acid substitution(s),
n. the V_{L} domain comprises:
   i. the CDRL3 amino acid sequence is as defined by IMGT or Kabat and is from a V_{L} domain selected from SEQ ID NO: 180, or wherein the CDRL3 amino acid sequence is as defined by IMGT or Kabat and is from a V_{L} domain selected from SEQ ID NO: 180 comprising 10, 9, 8, 7, 6, 5, 4, 3, 2 or 1 amino acid substitution(s); and/or
   ii. a CDRL1 amino acid sequence is as defined by IMGT or Kabat and is from a V_{L} domain selected from SEQ ID NO: 180, or wherein the CDRL1 amino acid sequence is as defined by IMGT or Kabat and is from a V_{L} domain selected from SEQ ID NO: 180 comprising 10, 9, 8, 7, 6, 5, 4, 3, 2 or 1 amino acid substitution(s); and/or
   iii. a CDRL2 amino acid sequence is as defined by IMGT or Kabat and is from a V_{L} domain selected from SEQ ID NO: 180, or wherein the CDRL2 amino acid sequence is as defined by IMGT or Kabat and is from a V_{L} domain selected from SEQ ID NO: 180 comprising 10, 9, 8, 7, 6, 5, 4, 3, 2 or 1 amino acid substitution(s),
o. the V_{L} domain comprises:
   i. the CDRL3 amino acid sequence is as defined by IMGT or Kabat and is from a V_{L} domain selected from SEQ ID NO: 188, or wherein the CDRL3 amino acid sequence is as defined by IMGT or Kabat and is from a V_{L} domain selected from SEQ ID NO: 188 comprising 10, 9, 8, 7, 6, 5, 4, 3, 2 or 1 amino acid substitution(s); and/or
   ii. a CDRL1 amino acid sequence is as defined by IMGT or Kabat and is from a V_{L} domain selected from SEQ ID NO: 188, or wherein the CDRL1 amino acid sequence is as defined by IMGT or Kabat and is from a V_{L} domain selected from SEQ ID NO: 188 comprising 10, 9, 8, 7, 6, 5, 4, 3, 2 or 1 amino acid substitution(s); and/or
   iii. a CDRL2 amino acid sequence is as defined by IMGT or Kabat and is from a V_{L} domain selected from SEQ ID NO: 188, or wherein the CDRL2 amino acid sequence is as defined by IMGT or Kabat and is from a V_{L} domain selected from SEQ ID NO: 188 comprising 10, 9, 8, 7, 6, 5, 4, 3, 2 or 1 amino acid substitution(s),
p. the V_{L} domain comprises:
   i. the CDRL3 amino acid sequence is as defined by IMGT or Kabat and is from a V_{L} domain selected from SEQ ID NO: 196, or wherein the CDRL3 amino acid sequence is as defined by IMGT or Kabat and is from a V_{L} domain selected from SEQ ID NO: 196 comprising 10, 9, 8, 7, 6, 5, 4, 3, 2 or 1 amino acid substitution(s); and/or
   ii. a CDRL1 amino acid sequence is as defined by IMGT or Kabat and is from a V_{L} domain selected from SEQ ID NO: 196, or wherein the CDRL1 amino acid sequence is as defined by IMGT or Kabat and is from a V_{L} domain selected from SEQ ID NO: 196 comprising 10, 9, 8, 7, 6, 5, 4, 3, 2 or 1 amino acid substitution(s); and/or
   iii. a CDRL2 amino acid sequence is as defined by IMGT or Kabat and is from a V_{L} domain selected from SEQ ID NO: 196, or wherein the CDRL2 amino acid sequence is as defined by IMGT or Kabat and is from a V_{L} domain selected from SEQ ID NO: 196 comprising 10, 9, 8, 7, 6, 5, 4, 3, 2 or 1 amino acid substitution(s),
q. the V_{L} domain comprises:
   i. the CDRL3 amino acid sequence is as defined by IMGT or Kabat and is from a V_{L} domain selected from SEQ ID NO: 204, or wherein the CDRL3 amino acid sequence is as defined by IMGT or Kabat and is from a V_{L} domain selected from SEQ ID NO: 204 comprising 10, 9, 8, 7, 6, 5, 4, 3, 2 or 1 amino acid substitution(s); and/or
   ii. a CDRL1 amino acid sequence is as defined by IMGT or Kabat and is from a V_{L} domain selected from SEQ ID NO: 204, or wherein the CDRL1 amino acid sequence is as defined by IMGT or Kabat and is from a V_{L} domain selected from SEQ ID NO: 204 comprising 10, 9, 8, 7, 6, 5, 4, 3, 2 or 1 amino acid substitution(s); and/or
   iii. a CDRL2 amino acid sequence is as defined by IMGT or Kabat and is from a V_{L} domain selected from SEQ ID NO: 204, or wherein the CDRL2 amino acid sequence is as defined by IMGT or Kabat and is from a V_{L} domain selected from SEQ ID NO: 204 comprising 10, 9, 8, 7, 6, 5, 4, 3, 2 or 1 amino acid substitution(s),
r. the V_{L} domain comprises:
   i. the CDRL3 amino acid sequence is as defined by IMGT or Kabat and is from a V_{L} domain selected from SEQ ID NO: 212, or wherein the CDRL3 amino acid sequence is as defined by IMGT or Kabat and is from a V_{L} domain selected from SEQ ID NO: 212 comprising 10, 9, 8, 7, 6, 5, 4, 3, 2 or 1 amino acid substitution(s); and/or
   ii. a CDRL1 amino acid sequence is as defined by IMGT or Kabat and is from a V_{L} domain selected from SEQ ID NO: 212, or wherein the CDRL1 amino acid sequence is as defined by IMGT or Kabat and is from a V_{L} domain selected from SEQ ID NO: 212 comprising 10, 9, 8, 7, 6, 5, 4, 3, 2 or 1 amino acid substitution(s); and/or
   iii. a CDRL2 amino acid sequence is as defined by IMGT or Kabat and is from a V_{L} domain selected from SEQ ID NO: 212, or wherein the CDRL2 amino acid sequence is as defined by IMGT or Kabat and is from a V_{L} domain selected from SEQ ID NO: 212 comprising 10, 9, 8, 7, 6, 5, 4, 3, 2 or 1 amino acid substitution(s),
s. the V_{L} domain comprises:
   i. the CDRL3 amino acid sequence is as defined by IMGT or Kabat and is from a V_{L} domain selected from SEQ ID NO: 220, or wherein the CDRL3 amino acid sequence is as defined by IMGT or Kabat and is from a V_{L} domain selected from SEQ ID NO: 220 comprising 10, 9, 8, 7, 6, 5, 4, 3, 2 or 1 amino acid substitution(s); and/or
   ii. a CDRL1 amino acid sequence is as defined by IMGT or Kabat and is from a V_{L} domain selected from SEQ ID NO: 220, or wherein the CDRL1 amino acid sequence is as defined by IMGT or Kabat and is from a V_{L} domain selected from SEQ ID NO: 220 comprising 10, 9, 8, 7, 6, 5, 4, 3, 2 or 1 amino acid substitution(s); and/or
   iii. a CDRL2 amino acid sequence is as defined by IMGT or Kabat and is from a V_{L} domain selected from SEQ ID NO: 220, or wherein the CDRL2 amino acid sequence is as defined by IMGT or Kabat and is from a V_{L} domain selected from SEQ ID NO: 220 comprising 10, 9, 8, 7, 6, 5, 4, 3, 2 or 1 amino acid substitution(s),
t. the V_{L} domain comprises:
   i. the CDRL3 amino acid sequence is as defined by IMGT or Kabat and is from a V_{L} domain selected from SEQ ID NO: 228, or wherein the CDRL3 amino acid sequence is as defined by IMGT or Kabat and is from a V_{L} domain selected from SEQ ID NO: 228 comprising 10, 9, 8, 7, 6, 5, 4, 3, 2 or 1 amino acid substitution(s); and/or
   ii. a CDRL1 amino acid sequence is as defined by IMGT or Kabat and is from a V_{L} domain selected from SEQ ID NO: 228, or wherein the CDRL1 amino acid sequence is as defined by IMGT or Kabat and is from a V_{L} domain selected from SEQ ID NO: 228 comprising 10, 9, 8, 7, 6, 5, 4, 3, 2 or 1 amino acid substitution(s); and/or
   iii. a CDRL2 amino acid sequence is as defined by IMGT or Kabat and is from a V_{L} domain selected from SEQ ID NO: 228, or wherein the CDRL2 amino acid sequence is as defined by IMGT or Kabat and is from a V_{L} domain selected from SEQ ID NO: 228 comprising 10, 9, 8, 7, 6, 5, 4, 3, 2 or 1 amino acid substitution(s), or
u. the V_{L} domain comprises:
   i. the CDRL3 amino acid sequence is as defined by IMGT or Kabat and is from a V_{L} domain selected from SEQ ID NO: 236, or wherein the CDRL3 amino acid sequence is as defined by IMGT or Kabat and is from a V_{L} domain selected from SEQ ID NO: 236 comprising 10, 9, 8, 7, 6, 5, 4, 3, 2 or 1 amino acid substitution(s); and/or
   ii. a CDRL1 amino acid sequence is as defined by IMGT or Kabat and is from a V_{L} domain selected from SEQ ID NO: 236, or wherein the CDRL1 amino acid sequence is as defined by IMGT or Kabat and is from a V_{L} domain selected from SEQ ID NO: 236 comprising 10, 9, 8, 7, 6, 5, 4, 3, 2 or 1 amino acid substitution(s); and/or
   iii. a CDRL2 amino acid sequence is as defined by IMGT or Kabat and is from a V_{L} domain selected from SEQ ID NO: 236, or wherein the CDRL2 amino acid sequence is as defined by IMGT or Kabat and is from a V_{L} domain selected from SEQ ID NO: 236 comprising 10, 9, 8, 7, 6, 5, 4, 3, 2 or 1 amino acid substitution(s).

According to the embodiment herein, the specified CDR (e.g. CDRH1, CDRH2, CDRH3, CDRL1, CDRL2 and/or CDRL3) is as defined by Kabat or IMGT. In one embodiment, the specified CDR comprises one amino acid substitution, optionally a conservative amino acid substitution. In one embodiment, the specified CDR comprises one or two (e.g. two) amino acid substitutions, optionally conservative amino acid substitutions. In one embodiment, the specified CDR comprises one, two or three (e.g. three, or two or three) amino acid substitutions, optionally conservative amino acid substitutions. In one embodiment, the specified CDR comprises one, two, three or four (e.g. four, three or four, one or four, or two or four) amino acid substitutions, optionally conservative amino acid substitutions. In one embodiment, the specified CDR comprises one, two, three, four or five (e.g. five, four or five, three or five, two or five, or one or five) amino acid substitutions, optionally conservative amino acid substitutions. In one embodiment, the specified CDR comprises one, two, three, four, five or six (e.g. six, five or six, four or six, three or six, two or six, or one or six) amino acid substitutions, optionally conservative amino acid substitutions. Amino acid substitutions include alterations in which an amino acid is replaced with a different naturally-occurring amino acid residue. Such substitutions may be classified as “conservative”, in which case an amino acid residue contained in a polypeptide is replaced with another naturally occurring amino acid of similar character either in relation to polarity, side chain functionality or size. Such conservative substitutions are well known in the art. In one embodiment, the amino acid substitutions are conservative amino acid substitutions, optionally wherein a conservative substitution is where an amino acid from group (1) to (8) is replaced with an amino acid in the same group:
1) Glycine (G), Alanine (A);
2) Serine (S), Threonine (T), Cysteine (C), Methionine (M);
3) Aspartic acid (D), Glutamic acid (E);
4) Asparagine (N), Glutamine (Q);
5) Aspartic acid (D), Glutamic acid (E), Asparagine (N), Glutamine (Q);
6) Arginine (R), Lysine (K), Histidine (H);
7) Glycine (G), Alanine (A), Isoleucine (I), Leucine (L), Valine (V);
8) Isoleucine (I), Leucine (L), Methionine (M), Valine (V) Proline (P); and
9) Phenylalanine (F), Tyrosine (Y), Tryptophan (W).

In one embodiment, the antibody or antigen-binding fragment comprises a V_{H} domain, wherein the V_{H} domain comprises a CDRH1 amino acid sequence of SEQ ID NO: 10 or 13, a CDRH2 amino acid sequence of SEQ ID NO: 11 or 14, and a CDRH3 amino acid sequence of SEQ ID NO: 12 or 15; and wherein the antibody or antigen-binding fragment comprises a V_{L} domain, wherein the V_{L} domain comprises a CDRL1 amino acid sequence of SEQ ID NO: 20 or 23, a CDRL2 amino acid sequence of SEQ ID NO: 21 or 24, and a CDRL3 amino acid sequence of SEQ ID NO: 22 or 25, wherein one or more CDRs (e.g. CDRH1, CDRH2, CDRH3, CDRL1, CDRL2 and/or CDRL3) comprises up to one amino acid substitution, optionally a conservative amino acid substitution.

In one embodiment, the antibody or antigen-binding fragment comprises a V_{H} domain, wherein the V_{H} domain comprises a CDRH1 amino acid sequence of SEQ ID NO: 13, a CDRH2 amino acid sequence of SEQ ID NO: 14, and a CDRH3 amino acid sequence of SEQ ID NO: 15; and wherein the antibody or antigen-binding fragment comprises a V_{L} domain, wherein the V_{L} domain comprises a CDRL1 amino acid sequence of SEQ ID NO: 23, a CDRL2 amino acid sequence of SEQ ID NO: 24, and a CDRL3 amino acid sequence of SEQ ID NO: 25, wherein one or more CDRs (i.e. CDRH1, CDRH2, CDRH3, CDRL1, CDRL2 and/or CDRL3) comprises up to one amino acid substitution, optionally a conservative amino acid substitution.

In one embodiment, the antibody or antigen-binding fragment comprises a V_{H} domain, wherein the V_{H} domain comprises a CDRH1 amino acid sequence of SEQ ID NO: 13, a CDRH2 amino acid sequence of SEQ ID NO: 14, and a CDRH3 amino acid sequence of SEQ ID NO: 15; and wherein the antibody or antigen-binding fragment comprises a V_{L} domain, wherein the V_{L} domain comprises a CDRL1 amino acid sequence of SEQ ID NO: 23, a CDRL2 amino acid sequence of SEQ ID NO: 24, and a CDRL3 amino acid sequence of SEQ ID NO: 25, wherein one CDR (i.e. CDRH1, CDRH2, CDRH3, CDRL1, CDRL2 or CDRL3) comprises up to one amino acid substitution, optionally a conservative amino acid substitution.

In one embodiment, the antibody or antigen-binding fragment comprises a V_{H} domain wherein
a. the V_{H} domain comprises an amino acid sequence which is identical or at least 90%, 95% or 98% identical to SEQ ID NO: 16;
b. the V_{H} domain comprises an amino acid sequence which is identical or at least 90%, 95% or 98% identical to SEQ ID NO: 36;
c. the V_{H} domain comprises an amino acid sequence which is identical or at least 90%, 95% or 98% identical to SEQ ID NO: 56;
d. the V_{H} domain comprises an amino acid sequence which is identical or at least 90%, 95% or 98% identical to SEQ ID NO: 76;
e. the V_{H} domain comprises an amino acid sequence which is identical or at least 90%, 95% or 98% identical to SEQ ID NO: 96;
f. the V_{H} domain comprises an amino acid sequence which is identical or at least 90%, 95% or 98% identical to SEQ ID NO: 112;
g. the V_{H} domain comprises an amino acid sequence which is identical or at least 90%, 95% or 98% identical to SEQ ID NO: 120;
h. the V_{H} domain comprises an amino acid sequence which is identical or at least 90%, 95% or 98% identical to SEQ ID NO: 128;
i. the V_{H} domain comprises an amino acid sequence which is identical or at least 90%, 95% or 98% identical to SEQ ID NO: 136;
j. the V_{H} domain comprises an amino acid sequence which is identical or at least 90%, 95% or 98% identical to SEQ ID NO: 144;
k. the V_{H} domain comprises an amino acid sequence which is identical or at least 90%, 95% or 98% identical to SEQ ID NO: 152;
l. the V_{H} domain comprises an amino acid sequence which is identical or at least 90%, 95% or 98% identical to SEQ ID NO: 160;
m. the V_{H} domain comprises an amino acid sequence which is identical or at least 90%, 95% or 98% identical to SEQ ID NO: 168;
n. the V_{H} domain comprises an amino acid sequence which is identical or at least 90%, 95% or 98% identical to SEQ ID NO: 176;
o. the V_{H} domain comprises an amino acid sequence which is identical or at least 90%, 95% or 98% identical to SEQ ID NO: 184;
p. the V_{H} domain comprises an amino acid sequence which is identical or at least 90%, 95% or 98% identical to SEQ ID NO: 192;
q. the V_{H} domain comprises an amino acid sequence which is identical or at least 90%, 95% or 98% identical to SEQ ID NO: 200;
r. the V_{H} domain comprises an amino acid sequence which is identical or at least 90%, 95% or 98% identical to SEQ ID NO: 208;
s. the V_{H} domain comprises an amino acid sequence which is identical or at least 90%, 95% or 98% identical to SEQ ID NO: 216;
t. the V_{H} domain comprises an amino acid sequence which is identical or at least 90%, 95% or 98% identical to SEQ ID NO: 224; or
u. the V_{H} domain comprises an amino acid sequence which is identical or at least 90%, 95% or 98% identical to SEQ ID NO: 232.

In one embodiment, the antibody or antigen-binding fragment comprises, or further comprises, a V_{L} domain wherein
a. the V_{L} domain comprises an amino acid sequence which is identical or at least 90%, 95% or 98% identical to SEQ ID NO: 26;
b. the V_{L} domain comprises an amino acid sequence which is identical or at least 90%, 95% or 98% identical to SEQ ID NO: 46;
c. the V_{L} domain comprises an amino acid sequence which is identical or at least 90%, 95% or 98% identical to SEQ ID NO: 66;
d. the V_{L} domain comprises an amino acid sequence which is identical or at least 90%, 95% or 98% identical to SEQ ID NO: 86;
e. the V_{L} domain comprises an amino acid sequence which is identical or at least 90%, 95% or 98% identical to SEQ ID NO: 106;
f. the V_{L} domain comprises an amino acid sequence which is identical or at least 90%, 95% or 98% identical to SEQ ID NO: 116;
g. the V_{L} domain comprises an amino acid sequence which is identical or at least 90%, 95% or 98% identical to SEQ ID NO: 124;
h. the V_{L} domain comprises an amino acid sequence which is identical or at least 90%, 95% or 98% identical to SEQ ID NO: 132;
i. the V_{L} domain comprises an amino acid sequence which is identical or at least 9080%, 95% or 98% identical to SEQ ID NO: 140;
j. the V_{L} domain comprises an amino acid sequence which is identical or at least 90%, 95% or 98% identical to SEQ ID NO: 148;
k. the V_{L} domain comprises an amino acid sequence which is identical or at least 90%, 95% or 98% identical to SEQ ID NO: 156;
l. the V_{L} domain comprises an amino acid sequence which is identical or at least 90%, 95% or 98% identical to SEQ ID NO: 164;
m. the V_{L} domain comprises an amino acid sequence which is identical or at least 90%, 95% or 98% identical to SEQ ID NO: 172;
n. the V_{L} domain comprises an amino acid sequence which is identical or at least 90%, 95% or 98% identical to SEQ ID NO: 180;
o. the V_{L} domain comprises an amino acid sequence which is identical or at least 90%, 95% or 98% identical to SEQ ID NO: 188;
p. the V_{L} domain comprises an amino acid sequence which is identical or at least 90%, 95% or 98% identical to SEQ ID NO: 196;
q. the V_{L} domain comprises an amino acid sequence which is identical or at least 90%, 95% or 98% identical to SEQ ID NO: 204;
r. the V_{L} domain comprises an amino acid sequence which is identical or at least 90%, 95% or 98% identical to SEQ ID NO: 212;
s. the V_{L} domain comprises an amino acid sequence which is identical or at least 90%, 95% or 98% identical to SEQ ID NO: 220;
t. the V_{L} domain comprises an amino acid sequence which is identical or at least 90%, 95% or 98% identical to SEQ ID NO: 228; or
u. the V_{L} domain comprises an amino acid sequence which is identical or at least 90%, 95% or 98% identical to SEQ ID NO: 236.

For the avoidance of doubt, the term “at least X % identical” as used throughout these embodiments is intended to refer to a “% identity” as defined herein.

In one embodiment, the antibody or antigen-binding fragment comprises a V_{H} domain and a V_{L} domain, wherein
a. the V_{H} domain comprises an amino acid sequence which is identical or at least 90%, 95% or 98% identical to SEQ ID NO: 16; and the V_{L} domain comprises an amino acid sequence which is identical or at least 90%, 95% or 98% identical to SEQ ID NO: 26;
b. the V_{H} domain comprises an amino acid sequence which is identical or at least 90%, 95% or 98% identical to SEQ ID NO: 36; and the V_{L} domain comprises an amino acid sequence which is identical or at least 90%, 95% or 98% identical to SEQ ID NO: 46;
c. the V_{H} domain comprises an amino acid sequence which is identical or at least 90%, 95% or 98% identical to SEQ ID NO: 56; and the V_{L} domain comprises an amino acid sequence which is identical or at least 90%, 95% or 98% identical to SEQ ID NO: 66;
d. the V_{H} domain comprises an amino acid sequence which is identical or at least 90%, 95% or 98% identical to SEQ ID NO: 76; and the V_{L} domain comprises an amino acid sequence which is identical or at least 90%, 95% or 98% identical to SEQ ID NO: 86;
e. the V_{H} domain comprises an amino acid sequence which is identical or at least 90%, 95% or 98% identical to SEQ ID NO: 96; and the V_{L} domain comprises an amino acid sequence which is identical or at least 90%, 95% or 98% identical to SEQ ID NO: 106;
f. the V_{H} domain comprises an amino acid sequence which is identical or at least 90%, 95% or 98% identical to SEQ ID NO: 112; and the V_{L} domain comprises an amino acid sequence which is identical or at least 90%, 95% or 98% identical to SEQ ID NO: 116;
g. the V_{H} domain comprises an amino acid sequence which is identical or at least 90%, 95% or 98% identical to SEQ ID NO: 120; and the V_{L} domain comprises an amino acid sequence which is identical or at least 90%, 95% or 98% identical to SEQ ID NO: 124;
h. the V_{H} domain comprises an amino acid sequence which is identical or at least 90%, 95% or 98% identical to SEQ ID NO: 128; and the V_{L} domain comprises an amino acid sequence which is identical or at least 90%, 95% or 98% identical to SEQ ID NO: 132;
i. the V_{H} domain comprises an amino acid sequence which is identical or at least 90%, 95% or 98% identical to SEQ ID NO: 136; and the V_{L} domain comprises an amino acid sequence which is identical or at least 90%, 95% or 98% identical to SEQ ID NO: 140;
j. the V_{H} domain comprises an amino acid sequence which is identical or at least 90%, 95% or 98% identical to SEQ ID NO: 144; and the V_{L} domain comprises an amino acid sequence which is identical or at least 90%, 95% or 98% identical to SEQ ID NO: 148;
k. the V_{H} domain comprises an amino acid sequence which is identical or at least 90%, 95% or 98% identical to SEQ ID NO: 152; and the V_{L} domain comprises an amino acid sequence which is identical or at least 90%, 95% or 98% identical to SEQ ID NO: 156;
l. the V_{H} domain comprises an amino acid sequence which is identical or at least 90%, 95% or 98% identical to SEQ ID NO: 160; and the V_{L} domain comprises an amino acid sequence which is identical or at least 90%, 95% or 98% identical to SEQ ID NO: 164;
m. the V_{H} domain comprises an amino acid sequence which is identical or at least 90%, 95% or 98% identical to SEQ ID NO: 168; and the V_{L} domain comprises an amino acid sequence which is identical or at least 90%, 95% or 98% identical to SEQ ID NO: 172;
n. the V_{H} domain comprises an amino acid sequence which is identical or at least 90%, 95% or 98% identical to SEQ ID NO: 176; and the V_{L} domain comprises an amino acid sequence which is identical or at least 90%, 95% or 98% identical to SEQ ID NO: 180;
o. the V_{H} domain comprises an amino acid sequence which is identical or at least 90%, 95% or 98% identical to SEQ ID NO: 184; and the V_{L} domain comprises an amino acid sequence which is identical or at least 90%, 95% or 98% identical to SEQ ID NO: 188;
p. the V_{H} domain comprises an amino acid sequence which is identical or at least 90%, 95% or 98% identical to SEQ ID NO: 192; and the V_{L} domain comprises an amino acid sequence which is identical or at least 90%, 95% or 98% identical to SEQ ID NO: 196;
q. the V_{H} domain comprises an amino acid sequence which is identical or at least 90%, 95% or 98% identical to SEQ ID NO: 200; and the V_{L} domain comprises an amino acid sequence which is identical or at least 90%, 95% or 98% identical to SEQ ID NO: 204;
r. the V_{H} domain comprises an amino acid sequence which is identical or at least 90%, 95% or 98% identical to SEQ ID NO: 208; and the V_{L} domain comprises an amino acid sequence which is identical or at least 90%, 95% or 98% identical to SEQ ID NO: 212;
s. the V_{H} domain comprises an amino acid sequence which is identical or at least 90%, 95% or 98% identical to SEQ ID NO: 216; and the V_{L} domain comprises an amino acid sequence which is identical or at least 90%, 95% or 98% identical to SEQ ID NO: 220;
t. the V_{H} domain comprises an amino acid sequence which is identical or at least 90%, 95% or 98% identical to SEQ ID NO: 224; and the V_{L} domain comprises an amino acid sequence which is identical or at least 90%, 95% or 98% identical to SEQ ID NO: 228; or
u. the V_{H} domain comprises an amino acid sequence which is identical or at least 90%, 95% or 98% identical to SEQ ID NO: 232; and the V_{L} domain comprises an amino acid sequence which is identical or at least 90%, 95% or 98% identical to SEQ ID NO: 236.

In one embodiment the antibody or antigen-binding fragment comprises a heavy chain and a light chain, wherein
a. the heavy chain amino acid sequence comprises an amino acid sequence of SEQ ID NO: 18, and the light chain amino acid sequence comprises an amino acid sequence of SEQ ID NO: 28;
b. the heavy chain amino acid sequence comprises an amino acid sequence that is at least 90%, 95% or 98% identical to SEQ ID NO: 18, and the light chain amino acid sequence comprises an amino acid sequence that is at least 90%, 95% or 98% identical to SEQ ID NO: 28;
c. the heavy chain amino acid sequence comprises an amino acid sequence of SEQ ID NO: 38, and the light chain amino acid sequence comprises an amino acid sequence of SEQ ID NO: 48;
d. the heavy chain amino acid sequence comprises an amino acid sequence that is at least 90%, 95% or 98% identical to SEQ ID NO: 38, and the light chain amino acid sequence comprises an amino acid sequence that is at least 90%, 95% or 98% identical to SEQ ID NO: 48;
e. the heavy chain amino acid sequence comprises an amino acid sequence of SEQ ID NO: 58, and the light chain amino acid sequence comprises an amino acid sequence of SEQ ID NO: 68;
f. the heavy chain amino acid sequence comprises an amino acid sequence that is at least 90%, 95% or 98% identical to SEQ ID NO: 58, and the light chain amino acid sequence comprises an amino acid sequence that is at least 90%, 95% or 98% identical to SEQ ID NO: 68;
g. the heavy chain amino acid sequence comprises an amino acid sequence of SEQ ID NO: 78, and the light chain amino acid sequence comprises an amino acid sequence of SEQ ID NO: 88;
h. the heavy chain amino acid sequence comprises an amino acid sequence that is at least 90%, 95% or 98% identical to SEQ ID NO: 78, and the light chain amino acid sequence comprises an amino acid sequence that is at least 90%, 95% or 98% identical to SEQ ID NO: 88;
i. the heavy chain amino acid sequence comprises an amino acid sequence of SEQ ID NO: 98, and the light chain amino acid sequence comprises an amino acid sequence of SEQ ID NO: 108;
j. the heavy chain amino acid sequence comprises an amino acid sequence that is at least 90%, 95% or 98% identical to SEQ ID NO: 98, and the light chain amino acid sequence comprises an amino acid sequence that is at least 90%, 95% or 98% identical to SEQ ID NO: 108;
k. the heavy chain amino acid sequence comprises an amino acid sequence of SEQ ID NO: 98, and the light chain amino acid sequence comprises an amino acid sequence of SEQ ID NO: 110;
l. the heavy chain amino acid sequence comprises an amino acid sequence that is at least 90%, 95% or 98% identical to SEQ ID NO: 98, and the light chain amino acid sequence comprises an amino acid sequence that is at least 90%, 95% or 98% identical to SEQ ID NO: 110;
m. the heavy chain amino acid sequence comprises an amino acid sequence of SEQ ID NO: 114, and the light chain amino acid sequence comprises an amino acid sequence of SEQ ID NO: 118;
n. the heavy chain amino acid sequence comprises an amino acid sequence that is at least 90%, 95% or 98% identical to SEQ ID NO: 114, and the light chain amino acid sequence comprises an amino acid sequence that is at least 90%, 95% or 98% identical to SEQ ID NO: 118;
o. the heavy chain amino acid sequence comprises an amino acid sequence of SEQ ID NO: 122, and the light chain amino acid sequence comprises an amino acid sequence of SEQ ID NO: 126;
p. the heavy chain amino acid sequence comprises an amino acid sequence that is at least 90%, 95% or 98% identical to SEQ ID NO: 122, and the light chain amino acid sequence comprises an amino acid sequence that is at least 90%, 95% or 98% identical to SEQ ID NO: 126;
q. the heavy chain amino acid sequence comprises an amino acid sequence of SEQ ID NO: 130, and the light chain amino acid sequence comprises an amino acid sequence of SEQ ID NO: 134;
r. the heavy chain amino acid sequence comprises an amino acid sequence that is at least 90%, 95% or 98% identical to SEQ ID NO: 130, and the light chain amino acid sequence comprises an amino acid sequence that is at least 90%, 95% or 98% identical to SEQ ID NO: 134;
s. the heavy chain amino acid sequence comprises an amino acid sequence of SEQ ID NO: 138, and the light chain amino acid sequence comprises an amino acid sequence of SEQ ID NO: 142;
t. the heavy chain amino acid sequence comprises an amino acid sequence that is at least 90%, 95% or 98% identical to SEQ ID NO: 138, and the light chain amino acid sequence comprises an amino acid sequence that is at least 90%, 95% or 98% identical to SEQ ID NO: 142;
u. the heavy chain amino acid sequence comprises an amino acid sequence of SEQ ID NO: 146, and the light chain amino acid sequence comprises an amino acid sequence of SEQ ID NO: 150;
v. the heavy chain amino acid sequence comprises an amino acid sequence that is at least 90%, 95% or 98% identical to SEQ ID NO: 146, and the light chain amino acid sequence comprises an amino acid sequence that is at least 90%, 95% or 98% identical to SEQ ID NO: 150;
w. the heavy chain amino acid sequence comprises an amino acid sequence of SEQ ID NO: 154, and the light chain amino acid sequence comprises an amino acid sequence of SEQ ID NO: 158;
x. the heavy chain amino acid sequence comprises an amino acid sequence that is at least 90%, 95% or 98% identical to SEQ ID NO: 154, and the light chain amino acid sequence comprises an amino acid sequence that is at least 90%, 95% or 98% identical to SEQ ID NO: 158;
y. the heavy chain amino acid sequence comprises an amino acid sequence of SEQ ID NO: 162, and the light chain amino acid sequence comprises an amino acid sequence of SEQ ID NO: 166;
z. the heavy chain amino acid sequence comprises an amino acid sequence that is at least 90%, 95% or 98% identical to SEQ ID NO: 162, and the light chain amino acid sequence comprises an amino acid sequence that is at least 90%, 95% or 98% identical to SEQ ID NO: 166;
aa. the heavy chain amino acid sequence comprises an amino acid sequence of SEQ ID NO: 170, and the light chain amino acid sequence comprises an amino acid sequence of SEQ ID NO: 174;
bb. the heavy chain amino acid sequence comprises an amino acid sequence that is at least 90%, 95% or 98% identical to SEQ ID NO: 170, and the light chain amino acid sequence comprises an amino acid sequence that is at least 90%, 95% or 98% identical to SEQ ID NO: 174;
cc. the heavy chain amino acid sequence comprises an amino acid sequence of SEQ ID NO: 178, and the light chain amino acid sequence comprises an amino acid sequence of SEQ ID NO: 182;
dd. the heavy chain amino acid sequence comprises an amino acid sequence that is at least 90%, 95% or 98% identical to SEQ ID NO: 178, and the light chain amino acid sequence comprises an amino acid sequence that is at least 90%, 95% or 98% identical to SEQ ID NO: 182;
ee. the heavy chain amino acid sequence comprises an amino acid sequence of SEQ ID NO: 186, and the light chain amino acid sequence comprises an amino acid sequence of SEQ ID NO: 190;
ff. the heavy chain amino acid sequence comprises an amino acid sequence that is at least 90%, 95% or 98% identical to SEQ ID NO: 186, and the light chain amino acid sequence comprises an amino acid sequence that is at least 90%, 95% or 98% identical to SEQ ID NO: 190;
gg. the heavy chain amino acid sequence comprises an amino acid sequence of SEQ ID NO: 194, and the light chain amino acid sequence comprises an amino acid sequence of SEQ ID NO: 198;
hh. the heavy chain amino acid sequence comprises an amino acid sequence that is at least 90%, 95% or 98% identical to SEQ ID NO: 194, and the light chain amino acid sequence comprises an amino acid sequence that is at least 90%, 95% or 98% identical to SEQ ID NO: 198;
ii. the heavy chain amino acid sequence comprises an amino acid sequence of SEQ ID NO: 202, and the light chain amino acid sequence comprises an amino acid sequence of SEQ ID NO: 206;
jj. the heavy chain amino acid sequence comprises an amino acid sequence that is at least 90%, 95% or 98% identical to SEQ ID NO: 202, and the light chain amino acid sequence comprises an amino acid sequence that is at least 90%, 95% or 98% identical to SEQ ID NO: 206;
kk. the heavy chain amino acid sequence comprises an amino acid sequence of SEQ ID NO: 210, and the light chain amino acid sequence comprises an amino acid sequence of SEQ ID NO: 214;
ll. the heavy chain amino acid sequence comprises an amino acid sequence that is at least 90%, 95% or 98% identical to SEQ ID NO: 210, and the light chain amino acid sequence comprises an amino acid sequence that is at least 90%, 95% or 98% identical to SEQ ID NO: 214;
mm. the heavy chain amino acid sequence comprises an amino acid sequence of SEQ ID NO: 218, and the light chain amino acid sequence comprises an amino acid sequence of SEQ ID NO: 222;
nn. the heavy chain amino acid sequence comprises an amino acid sequence that is at least 90%, 95% or 98% identical to SEQ ID NO: 218, and the light chain amino acid sequence comprises an amino acid sequence that is at least 90%, 95% or 98% identical to SEQ ID NO: 222;
oo. the heavy chain amino acid sequence comprises an amino acid sequence of SEQ ID NO: 226, and the light chain amino acid sequence comprises an amino acid sequence of SEQ ID NO: 230;
pp. the heavy chain amino acid sequence comprises an amino acid sequence that is at least 90%, 95% or 98% identical to SEQ ID NO: 226, and the light chain amino acid sequence comprises an amino acid sequence that is at least 90%, 95% or 98% identical to SEQ ID NO: 230;
qq. the heavy chain amino acid sequence comprises an amino acid sequence of SEQ ID NO: 234, and the light chain amino acid sequence comprises an amino acid sequence of SEQ ID NO: 238; or
rr. the heavy chain amino acid sequence comprises an amino acid sequence that is at least 90%, 9585% or 98% identical to SEQ ID NO: 234, and the light chain amino acid sequence comprises an amino acid sequence that is at least 90%, 95% or 98% identical to SEQ ID NO: 238.

In any of the embodiments described herein, the % identity may be substituted for any of the % identities selected from: 70% identical, 75% identical, 80% identical, 85% identical or 90% identical. In any of the embodiments herein, the % identity may be substituted for any of the % identities selected from: 91% identical, 92% identical, 93% identical, 94% identical or 95% identical, in particular 95% identical. In any of the embodiments herein, the % identity may be substituted for any of the % identities selected from: 96% identical, 97% identical, 98% identical, 99% identical or 100% identical, in particular 98% identical.

In one embodiment, the anti-CXCR4 antibodies or antigen-binding fragments described herein comprise a V_{H} domain comprising a CDRH3 which has a length of from 5 to 27 amino acids, from 5 to 25 amino acids, from 5 to 24 amino acids, from 5 to 23 amino acids, from 5 to 21 amino acids, from 10 to 25 amino acids, from 10 to 24 amino acids, from 10 to 23 amino acids, from 10 to 21 amino acids, from 12 to 25 amino acids, from 12 to 23 amino acids, from 12 to 21 amino acids, from 14 to 25 amino acids, from 14 to 23 amino acids or from 14 to 21 amino acids. In another embodiment the CDRH3 is 6, 14, 15, 17, 19, 21 or 23 amino acids in length.

In one embodiment, the anti-CXCR4 antibodies or antigen-binding fragments described herein comprise a V_{H} domain derived from the recombination of a human V_{H} gene segment, a human D gene segment and a human J_{H} gene segment, wherein the human V_{H} gene segment is IGHV3-33 (e.g. IGHV3-33*01), and/or the human D gene segment is IGHD1-20 (e.g. IGHD1-20*01), and/or the human J_{H} gene segment is IGHJ3 (e.g. IGHJ3*02), optionally wherein the antibody or antigen-binding fragment comprises a V_{H} domain comprising a CDRH3 which has a length of from 12 to 23 amino acids.

In one embodiment, the anti-CXCR4 antibodies or antigen-binding fragments described herein comprise a V_{H} domain derived from the recombination of a human V_{H} gene segment, a human D gene segment and a human J_{H} gene segment, wherein the human V_{H} gene segment is IGHV3-30 (e.g. IGHV3-30*18), and/or the human D gene segment is IGHD3-16 (e.g. IGHD3-16*02), and/or the human J_{H} gene segment is IGHJ6 (e.g. IGHJ6*02), optionally wherein the antibody or antigen-binding fragment comprises a V_{H} domain comprising a CDRH3 which has a length of from 12 to 23 amino acids.

In one embodiment, the anti-CXCR4 antibodies or antigen-binding fragments described herein comprise a V_{H} domain derived from the recombination of a human V_{H} gene segment, a human D gene segment and a human J_{H} gene segment, wherein the human V_{H} gene segment is IGHV3-73 (e.g. IGHV3-73*02), and/or the human D gene segment is IGHD3-9 (e.g. IGHD3-9*01), and/or the human J_{H} gene segment is IGHJ6 (e.g. IGHJ6*02), optionally wherein the antibody or antigen-binding fragment comprises a V_{H} domain comprising a CDRH3 which has a length of from 12 to 23 amino acids.

In one embodiment, the anti-CXCR4 antibodies or antigen-binding fragments described herein comprise a V_{H} domain derived from the recombination of a human V_{H} gene segment, a human D gene segment and a human J_{H} gene segment, wherein the human V_{H} gene segment is IGHV4-31 (e.g. IGHV4-31*03), and/or the human D gene segment is IGHD3-10 (e.g. IGHD3-10*01), and/or the human J_{H} gene segment is IGHJ6 (e.g. IGHJ6*02), optionally wherein the antibody or antigen-binding fragment comprises a V_{H} domain comprising a CDRH3 which has a length of from 12 to 23 amino acids.

In one embodiment, the anti-CXCR4 antibodies or antigen-binding fragments described herein comprise a V_{H} domain derived from the recombination of a human V_{H} gene segment, a human D gene segment and a human J_{H} gene segment, wherein the human V_{H} gene segment is IGHV3-23 (e.g. IGHV3-23*04), and/or the human D gene segment is IGHD1-1 (e.g. IGHD1-1*01), and/or the human J_{H} gene segment is IGHJ6 (e.g. IGHJ6*02), optionally wherein the antibody or antigen-binding fragment comprises a V_{H} domain comprising a CDRH3 which has a length of from 12 to 23 amino acids.

In one embodiment, the anti-CXCR4 antibodies or antigen-binding fragments described herein comprise a V_{H} domain comprising a CDRH3, wherein the CDRH3 comprises the amino acid motif AX₁GQLERR or X₂YGX₃DV, wherein X₁, X₂ and X₃ are independently any amino acid and optionally wherein the CDRH3 has a length of from 12 to 23 amino acids. In one embodiment, X₁ is an amino acid selected from histidine, lysine and arginine (preferably selected from lysine and arginine). In one embodiment, X₂ is an amino acid selected from serine, threonine and cysteine; or an amino acid selected from phenylalanine, tyrosine and tryptophan (preferably selected from serine and tyrosine). In one embodiment, X₃ is an amino acid selected from valine, leucine, isoleucine, methionine or proline (preferably selected from methionine or leucine). In one embodiment, the amino acid substitution is a conservative substitution(as described elsewhere herein).

In one embodiment, the anti-CXCR4 antibodies or antigen-binding fragments described herein comprise a V_{L} domain which comprises a CDRL3 comprising the motif LQX₁NX₂YPX₃T, wherein X₁, X₂ and X₃ are independently any amino acid. In one embodiment, X₁ is an amino acid selected from alanine or glycine, or an amino acid selected from serine, threonine or cysteine. In one embodiment, X₂ is an amino acid selected from asparagine or glutamine, or an amino acid selected from histidine, lysine or arginine. In one embodiment, X₃ is an amino acid selected from tyrosine, tryptophan or phenylalanine, or an amino acid selected from valine, leucine, isoleucine, methionine or proline. In one embodiment, the amino acid substitution is a conservative substitution (as described elsewhere herein).

In one embodiment, the anti-CXCR4 antibodies or antigen-binding fragments described herein compete with an antibody or antigen-binding fragment selected from CL-82574, CL-82458, CL-82558, CL-82658, CL-83083, CL-83083-2, CL-82583, CL-82580, CL-82577, CL-82571, CL-82562, CL-82556, CL-82551, CL-82541, CL-82523, CL-82517, CL-82495, CL-82473, CL-82455, CL-83158, CL-148712 and CL-148729 (in particular CL-82574 or CL-83083-2) for binding to CXCR4. The antibodies CL-82574, CL-82458, CL-82558, CL-82658, CL-83083, CL-83083-2, CL-82583, CL-82580, CL-82577, CL-82571, CL-82562, CL-82556, CL-82551, CL-82541, CL-82523, CL-82517, CL-82495, CL-82473, CL-82455, CL-83158, CL-148712 and CL-148729 are as described hereinabove, having any of the sequences of CDRs, variable regions and/or full length heavy and light chains as described. In one embodiment, competition is determined by SPR as described elsewhere herein. Such competition may be due, for example, to the antibodies or antigen-binding fragments binding to identical or overlapping epitopes of CXCR4. In another embodiment, competition is determined by ELISA, HTRF, fluorescence activated cell sorting (FACS) or such techniques being readily apparent to the skilled person and are described elsewhere herein. In one embodiment, competition is determined by Bio-Layer Interferometry (BLI) such techniques being readily apparent to the skilled person. In one embodiment, the antibody or antigen-binding fragment competes (e.g., in a dose-dependent manner) with CXCL12 (or a fusion protein thereof) for binding to cell surface-expressed CXCR4.

In one embodiment, the anti-CXCR4 antibodies or antigen-binding fragments described herein bind to an epitope (that is identical to an epitope) to which an antibody or antigen-binding fragment selected from CL-82574, CL-82458, CL-82558, CL-82658, CL-83083, CL-83083-2, CL-82583, CL-82580, CL-82577, CL-82571, CL-82562, CL-82556, CL-82551, CL-82541, CL-82523, CL-82517, CL-82495, CL-82473, CL-82455, CL-83158, CL-148712 and CL-148729 (in particular CL-82574 or CL-83083-2) specifically binds. In one embodiment, the epitope is identified by site-directed mutagenesis (e.g. alanine scanning), overlapping peptide scan (e.g. pepscan) or by X-ray crystallography. In one embodiment, there is provided an antibody or antigen-binding fragment which specifically binds to an epitope which is (substantially) similar (or identical) to an epitope to which any of the antibodies CL-82574, CL-82458, CL-82558, CL-82658, CL-83083, CL-83083-2, CL-82583, CL-82580, CL-82577, CL-82571, CL-82562, CL-82556, CL-82551, CL-82541, CL-82523, CL-82517, CL-82495, CL-82473, CL-82455, CL-83158, CL-148712 and CL-148729 specifically binds. Contact amino acid residues involved in the interaction of antibody or antigen-binding fragment and antigen may be determined by various known methods to those skilled in the art. In one embodiment, sequential replacement of the amino acids of the antigen sequence (using standard molecular biology techniques to mutate the DNA of the coding sequence of the antigen), in this case CXCR4 with alanine (a.k.a. alanine scan), or another unrelated amino acid, may provide residues whose mutation would reduce or ablate the ability of the antibody or antigen-binding fragment to recognise the antigen in question. In one embodiment, binding is assessed using standard techniques, such as, but not limited to, SPR, HTRF, ELISA (which are described elsewhere herein). Other substitutions could be made to enhance the disruption of binding such as changing the charge on the side chain of antigen sequence amino acids (e.g. lysine change to glutamic acid), switching polar and non-polar residues (e.g. serine change to leucine). The alanine scan or other amino acid substitution method may be carried out directly on cells using transient or stable expression of the mutated versions. When the alanine scan or other amino acid substitution method is carried out with either ELISA or HTRF, an amino acid residue is identified as contributing to the epitope if the reduction in signal is at least 25% or 30%. In one embodiment, the reduction in signal is at least 35%, at least 40%, at least 45%, at least 50%, at least 55% or at least 60%. In one embodiment, the reduction in signal is at least 70%, at least 75%, at least 80%, at least 85% or at least 90%. When the alanine scan or other amino acid substitution method is carried out with SPR, an amino acid residue is identified as contributing to the epitope if there is at least a 10-fold, at least 15-fold, at least 20-fold, at least 30-fold, at least 40-fold, at least 50-fold, or at least 100-fold. In one embodiment, X-ray crystallography is used to determine contact residues between antibody or antigen-binding fragment and antigen (i.e. to determine the epitope to which the antibody or antigen-binding fragment binds), crystallography allows the direct visualisation of contact residues involved in the antibody-antigen interaction; optionally wherein X-ray crystallography uses a thermostabilised receptor. In one embodiment, if the antibody or antigen-binding fragment recognises a linear epitope, short peptides based on the antigen sequence can be produced and binding of the antibody or antigen-binding fragment to these peptides can be assessed using standard techniques (e.g. SPR, HTRF or ELISA). Further investigation of the epitope could be provided by performing an Alanine scan on any peptides that show binding. Alternative to linear peptides, conformational scans (including overlapping peptide scan) could be carried out using Pepscan technology (http://www.pepscan.com/) using their chemical linkage of peptides onto scaffolds, which has been used to determine discontinuous epitopes on CD20 targeting antibodies (Niederfellner, Gerhard, et al., “Epitope characterization and crystal structure of GA101 provide insights into the molecular basis for type I/II distinction of CD20 antibodies.”, Blood, 118.2, (2011), 358-367.).

In one embodiment, the polypeptide (e.g. CXCR4) is a monomer. In another embodiment, the polypeptide (e.g. CXCR4) forms a homodimer with another identical polypeptide. In another embodiment, the polypeptide (e.g. CXCR4) forms a heterodimer with a different polypeptide (e.g. CXCR1, CXCR2, CXCR3, CXCR5, CXCR6, CXCR7, CCR1, CCR2, CCR3, CCR4, CCR5, CCR6, CCR7, CCR8, CCR9, CCR10, CB2, ADCYAP1R1, ADORA2B, ADORA3, ADRB2, APLNR, C5AR1, CALCR, CHRM1, GALR1, EDNRB, HRH1, MLNR, NTSR1, PTGER2, PTGER3, SSTR2, or TACR3). In one embodiment, the polypeptide (e.g. CXCR4) forms a heterodimer with CXCR7 or CXCR3. In another embodiment, the polypeptide (e.g. CXCR4) forms a heterodimer with CCR5 or CB2. In another embodiment, the polypeptide (e.g. CXCR4) forms a trimer or a tetramer. In another embodiment, the polypeptide (e.g. CXCR4) forms a multimer, which is an association of more than two polypeptides. A multimer consists of multiple identical polypeptides or a combination of different polypeptides or both (e.g. multiple CXCR4 proteins; or two or more CXCR4 proteins and one or more CXCR7 proteins). Dimerisation or oligomerisation may influence the receptors structure or function (e.g. signal transduction, signalling profile). In one embodiment, dimerisation can be determined by proximity-based assay is selected from the group consisting of bimolecular fluorescence complementation (BiFC), proximity ligation assay (PLA), fluorescence resonance energy transfer (FRET), bioluminescence resonance energy transfer (BRET), cysteine crosslinking, and co-immunoprecipitation.

In one embodiment, the anti-CXCR4 antibodies or antigen-binding fragments described herein (specifically or additionally) bind to CXCR4 homodimers. In one embodiment, the antibody or antigen-binding fragment (specifically or additionally) binds to a CXCR4 monomer. In one embodiment, the antibody or antigen-binding fragment (specifically or additionally) binds to a CXCR4 multimer. In one embodiment, the antibody or antigen-binding fragment (specifically or additionally) binds to a CXCR4 heterodimer. In one embodiment the heterodimer is a CXCR4 and CXCR7 heterodimer. In another embodiment, the heterodimer comprises CXCR4 and CXCR3, or CXCR4 and CXCR2. In another embodiment, the heterodimer comprises CXCR4 and another GPCR selected from; CXCR1, CXCR2, CXCR5 or CXCR6. In another embodiment, the heterodimer comprises CXCR4 and CCR5. In another embodiment, the antibody or antigen-binding fragment (specifically or additionally) binds to CXCR4 in complex with beta-arrestin.

In an embodiment, there is provided a nucleic acid which encodes a CDRH3 of an anti-CXCR4 antibody or antigen-binding fragment thereof as described herein.

In one embodiment, there is provided a nucleic acid that encodes a V_{H} domain and/or a V_{L} domain of an anti-CXCR4 antibody or antigen-binding fragment thereof as described herein. The V_{H} and V_{L} domain nucleic acid sequences of the invention are provided in the sequence listing.

In one embodiment, there is provided a nucleic acid comprises a nucleotide sequence that is at least 80%, 85% or 90% identical to the sequence(s) of
a. SEQ ID NO: 19 and/or SEQ ID NO: 29;
b. SEQ ID NO: 39 and/or SEQ ID NO: 49;
c. SEQ ID NO: 59 and/or SEQ ID NO: 69;
d. SEQ ID NO: 79 and/or SEQ ID NO: 89;
e. SEQ ID NO: 99 and/or SEQ ID NO: 109;
f. SEQ ID NO: 99 and/or SEQ ID NO: 111;
g. SEQ ID NO: 115 and/or SEQ ID NO: 119;
h. SEQ ID NO: 123 and/or SEQ ID NO: 127;
i. SEQ ID NO: 131 and/or SEQ ID NO: 135;
j. SEQ ID NO: 139 and/or SEQ ID NO: 143;
k. SEQ ID NO: 147 and/or SEQ ID NO: 151;
l. SEQ ID NO: 155 and/or SEQ ID NO: 159;
m. SEQ ID NO: 163 and/or SEQ ID NO: 167;
n. SEQ ID NO: 171 and/or SEQ ID NO: 175;
o. SEQ ID NO: 179 and/or SEQ ID NO: 183;
p. SEQ ID NO: 187 and/or SEQ ID NO: 191;
q. SEQ ID NO: 195 and/or SEQ ID NO: 199;
r. SEQ ID NO: 203 and/or SEQ ID NO: 207;
s. SEQ ID NO: 211 and/or SEQ ID NO: 215;
t. SEQ ID NO: 219 and/or SEQ ID NO: 223;
u. SEQ ID NO: 227 and/or SEQ ID NO: 231; or
v. SEQ ID NO: 235 and/or SEQ ID NO: 239.

In one embodiment, there is provided a nucleic acid encodes a heavy chain and/or a light chain of an anti-CXCR4 antibody or antigen-binding fragment thereof as described herein.

In any of the embodiments herein, the nucleic acid sequence is at least 70%, 75%, 80%, 85%, 90%, 91%, 93%, 95%, 96%, 97%, 98%, 99%, 99.5% or 99.9% identical to the specified sequence (e.g. as defined by a SEQ ID NO). In any of the embodiments herein, the nucleic acid is an isolated and purified nucleic acid.

In an embodiment, there is provided a vector which comprises a nucleic acid as described herein; optionally wherein the vector is a CHO or HEK293 vector.

In an embodiment, there is provided a host cell which comprises a nucleic acid described herein, or the vector as described herein. In one embodiment, the host cell is mammalian. In one embodiment, the host cell is a CHO (Chinese hamster ovary) cell (e.g. CHO-S cells) or a HEK (human embryonic kidney) cell (e.g. HEK293 or HEK293T cells).

### 3. Uses for Antibodies

### Therapeutic

Any of the anti-CXCR4 antibodies or antigen-binding fragments as described herein are useful for therapeutic modulation of the CXCL12/CXCR4 pathway, and hence are useful in treating or preventing CXCR4-mediated diseases or conditions.

Thus, in one embodiment, there is provided an anti-CXCR4 antibody or antigen-binding fragment as described herein for use in therapy. In one embodiment, there is provided a use of an anti-CXCR4 antibody or antigen-binding fragment described herein in the manufacture of a medicament for the therapy. In another embodiment, there is provided an anti-CXCR4 antibody or antigen-binding fragment as described herein for use in treating or preventing a CXCR4-mediated disease or condition as described hereinbelow.

In another embodiment, there is provided the use of an anti-CXCR4 antibody or antigen-binding fragment as described herein in the manufacture of a medicament for treating or preventing a CXCR4-mediated disease or condition as described hereinbelow.

In another embodiment, there is provided a method of treating or preventing a CXCR4-mediated disease or condition as described hereinbelow in a patient, comprising administering to said patient a therapeutically or prophylactically effective amount of an anti-CXCR4 antibody or antigen-binding fragment as described herein, wherein the CXCR4-mediated disease or condition is thereby treated or prevented.

In another embodiment, there is provided a pharmaceutical composition comprising an anti-CXCR4 antibody or antigen-binding fragment as described herein for use in therapy, or for use in treating or preventing CXCR4-mediated disease or condition as described hereinbelow.

Throughout this specification, a CXCR4-mediated disease or condition is any disease or condition which involves aberrant modulation of the CXCR4 pathway (e.g. CXCR4 signalling, downstream effects of CXCR4 and/or overexpression of CXCR4).

In a preferred embodiment, the CXCR4-mediated disease or condition is cancer, in particular a solid tumour. In a further embodiment, the cancer is characterised by the presence of CD8⁺ T-cells, for example T-cells that are unable to infiltrate the tumour (e.g. central tumour mass).

In one embodiment, the CXCR4-mediated disease or condition is one or more disease(s) or condition(s) independently selected from: small cell lung cancer, non-small cell lung cancer (e.g. adenocarcinoma, squamous cell carcinoma, and large cell carcinoma), breast cancer, ovarian cancer (e.g. metastatic ovarian cancer), renal carcinoma, stomach cancer, pancreatic cancer, pancreatic ductal cancer, colorectal cancer, cervical cancer, liver cancer, head and neck cancer, cerebral cancer, esophageal cancer, liver cancer, lung cancer, prostate cancer, uterus cancer, brain cancer, bladder cancer, sarcoma, adenocarcinoma, multiple myeloma, melanoma, glioma, astrocytoma (Grade I—Pilocytic Astrocytoma, Grade II—Low-grade Astrocytoma, Grade III—Anaplastic Astrocytoma, or Grade IV—Glioblastoma (GBM)), medulloblastoma, craniopharyngioma, ependymoma, pinealoma, hemangioblastoma, acoustic neuroma, oligodendroglioma, schwannoma, neurofibrosarcoma, osteosarcoma, meningioma, neuroblastoma, retinoblastoma, chordoma, CNS lymphoma, brain stem glioma, mixed glioma, optic nerve glioma, subependymoma, meningioma, metastatic brain tumour, oligodendroglioma, pituitary tumours, primitive neuroectodermal (PNET) tumour, juvenile pilocytic astrocytoma (JPA), pineal tumour, rhabdoid tumour and human papilloma virus (HPV)-associated cancers (wherein the HPV positive tumour and/or lesion is cervical intraepithelial neoplasia 1, cervical intraepithelial neoplasia 2, cervical intraepithelial neoplasia 3, cervical cancer (in situ and/or invasive), head and neck cancer, HPV papillomatosis, anal papillomatosis, anal cancer, vaginal cancer, vulvar cancer, and/or penile cancer).

In another embodiment, the CXCR4-mediated disease or condition is a cancer selected from carcinoma, sarcoma, myeloma, leukemia and/or lymphoma. In one embodiment, the CXCR4-mediated disease or condition is carcinoma, for example adenocarcinoma and/or squamous cell carcinoma. In one embodiment, the CXCR4-mediated disease or condition is adenocarcinoma (which develops in an organ or gland), for example one or more adenocarcinomas selected from colon cancer, breast cancer, lung cancer, prostate cancer, esophageal cancer, stomach cancer and pancreatic cancer. In one embodiment, the CXCR4-mediated disease or condition is squamous cell carcinoma, for example one or more carcinomas selected from melanoma, cervical cancer, head and neck cancer, vaginal cancer, thyroid cancer, esophageal cancer, lung cancer, penile cancer, bladder cancer, liver cancer and anal cancer. In one embodiment, the CXCR4-mediated disease or condition is a sarcoma, for example one or more sarcomas selected from bladder cancer, rhabdomyosarcoma, glioma and astrocytoma (e.g. Grade I—Pilocytic Astrocytoma, Grade II—Low-grade Astrocytoma, Grade III—Anaplastic Astrocytoma, or Grade IV—Glioblastoma (GBM)). In another embodiment, the CXCR4-mediated disease or condition is a myeloma, for example multiple myeloma. In another embodiment, the CXCR4-mediated disease or condition is a lymphoma, for example Hodgkin's and/or non-Hodgkin's lymphoma. In another embodiment, the CXCR4-mediated disease or condition is a leukaemia, for example one or more lymphomas selected from myelogenous leukemia, granulocytic leukemia, lymphatic leukemia, lymphocytic leukemia, lymphoblastic leukemia, polycythemia vera and erythremia. In another embodiment, the CXCR4-mediated disease or condition is a mixed type cancer (e.g. wherein the cancer comprises two or more different cancers), for example one or more mixed-type cancers selected from adenosquamous carcinoma, missed mesodermal tumour, carcinosarcoma and teratocarcinoma.

In one embodiment, the CXCR4-mediated disease or condition is one or more disease(s) or condition(s) independently selected from: ovarian cancer (e.g. metastatic ovarian cancer), pancreatic cancer, glioblastoma (GBM) and primitive neuroectodermal (PNET) tumour. In one embodiment, the CXCR4-mediated disease or condition is one or more disease(s) or condition(s) independently selected from: ovarian cancer (e.g. metastatic ovarian cancer), pancreatic cancer, glioblastoma (GBM), primitive neuroectodermal (PNET) tumour, small cell lung cancer, non-small cell lung cancer, breast cancer, renal cancer, pancreatic ductal cancer and colorectal cancer. In one embodiment, the CXCR4-mediated disease or condition is one or more disease(s) or condition(s) independently selected from: pancreatic cancer, pancreatic ductal cancer, prostate cancer and small cell lung cancer. In one embodiment, the CXCR4-mediated disease or condition is one or more disease(s) or condition(s) independently selected from: pancreatic cancer, pancreatic ductal cancer, prostate cancer, small cell lung cancer, glioma, melanoma, head and neck cancer, non-small cell lung cancer, colorectal cancer and oesophageal cancer.

In one embodiment, CXCR4-mediated diseases or conditions is one or more disease(s) or condition(s) independently selected from: Warts, Hypogammaglobulinemia, Infections, and Myelokathexis (WHIM) (also known as WHIM syndrome), Waldenstrom's Macroglobulinemia, Human Immunodeficiency Virus (HIV), thrombocytopaenia, ischemic heart failure, post myo-cardial infarction recovery, post myeloablative allogenic transplant, obliterative bronchiolitis, neutropoenia, malaria, opioid-induced hyperalgesia, rheumatoid arthritis, genetic disease of the blood, such as any of sickle cell anemia, hemophilia A, hemophilia B, alpha-thalassemia, beta-thalassemia, delta-thalassemia, von Willebrand Disease, pernicious anemia, Fanconi anemia, thrombocytopenic purpura, thrombophilia, and all primary immunodeficiency diseases. In one embodiment, CXCR4-mediated diseases or conditions include genetic disease of the blood, such as any of sickle cell anemia, hemophilia A, hemophilia B, alpha-thalassemia, beta-thalassemia, delta-thalassemia, von Willebrand Disease, pernicious anemia, Fanconi anemia, thrombocytopenic purpura, idiopathic pulmonary fibrosis and thrombophilia. In a preferred embodiment, the CXCR4-mediated disease or condition is WHIM or HIV.

In another embodiment, the anti-CXCR4 antibodies or antigen-binding fragments described herein are used in stem cell mobilisation (with or without granulocyte-colony stimulating factor) for the treatment of severe ischemic diseases, including myocardial infarction, limb ischemia, ischemic stroke, and acute kidney injury. In one embodiment, the CXCR4-mediated disease or condition is a post myeloablative allogenic transplant.

In another embodiment, the anti-CXCR4 antibodies or antigen-binding fragments disclosed herein are used in a method of endoradiotherapy. In another embodiment, the anti-CXCR4 antibodies or antigen-binding fragments disclosed herein are used in a method of wound healing.

In another embodiment, CXCR4-mediated diseases or conditions include CXCL12-mediated diseases or conditions. CXCL12-mediated diseases or conditions include any disease or condition which involves aberrant modulation of the CXCL12 pathway (e.g. CXCL12 signalling, downstream effects of CXCL12 and/or overexpression of CXCL12). CXCL12-mediated diseases or conditions include, but are not limited to, one or more selected from: germ cell tumours and metastasis (e.g. testicular germ cell tumours), pregnancy-associated diseases (e.g. preeclampsia) and adult onset stills disease.

In another embodiment, the anti-CXCR4 antibodies or antigen-binding fragments disclosed herein are used in combination with radiotherapy to reduce or treat the residual tumour, for example the antibodies or antigen-binding fragments are administered after radiotherapy (e.g. 12 h, 24 h, 48 h, 72 h, 96 h, 1 week or 2 weeks after radiotherapy). In one embodiment, the anti-CXCR4 antibodies or antigen-binding fragments disclosed herein are used in combination with radiotherapy to reduce or prevent vasculogenesis and/or tumour recurrence. This may be achieved by reducing or preventing infiltration of CXCR4⁺ cells (e.g. CXCR4⁺ immune cells; e.g. macrophages or T-cells) into an irradiated (via radiotherapy) tumour. In one embodiment, the residual tumour is the tumour mass remaining. In a one embodiment the tumour is any tumour listed in section ‘3. Uses for antibodies’. In a preferred embodiment, the tumour is glioblastoma and/or cervical cancer. Macrophage Exclusion after Radiation Therapy in Glioblastoma (MERT) is further described in Thomas et al (2019). Macrophage Exclusion after Radiation Therapy (MERT): A First in Human Phase I/II Trial using a CXCR4 Inhibitor in Glioblastoma. Clin Cancer Res. 25(23):6948-6957.

In one embodiment, there is provided a method of inhibiting CXCR4 activity in a patient or in vitro, comprising administering an effective amount of an anti-CXCR4 antibody or antigen-binding fragment as described herein.

In one embodiment, there is provided a method of reducing the size or volume of a (malignant) tumour in a patient, comprising administrating an effective amount of an anti-CXCR4 antibody or antigen-binding fragment as described herein to said patient.

In one embodiment, there is provided a method or use as described herein above which further comprises administering (to the patient) a further therapy, optionally wherein the further therapy comprises one or more (e.g. more than one, such as two or three) further therapeutic agents independently selected from the group consisting of:
a) immune checkpoint inhibitors (such as anti-PD-1 antibodies, anti-PD-L1 antibodies, anti-TIM-3 antibodies, anti-CTLA-4 antibodies, anti-TIGIT antibodies, anti-BTLA antibodies, anti-VISTA antibodies and anti-LAG-3 antibodies);
b) immune stimulators (such as anti-OX40 antibodies, anti-HVEM antibodies, anti-CD27 antibodies, anti-CD28 antibodies, anti-CD137L antibodies, anti-OX40L antibodies, anti-GITRL antibodies, anti-ICOSL antibodies, anti-PD-L2 antibodies, anti-GITR antibodies, anti-CD137 antibodies, anti-ICOS antibodies and anti-CD40 antibodies);
c) anti-CSF1R antibodies, anti-CCR4 antibodies, anti-CD39 antibodies, anti-CD73 antibodies, anti-CD96 antibodies, anti-CXCR2 antibodies, anti-CD200 antibodies, anti-GARP antibodies, anti-SIRPα antibodies, anti-CXCL9 antibodies, anti-CXCL10 antibodies, anti-CXCL11 antibodies and anti-CD155 antibodies;
d) chemokine receptor antagonists (such as CCR4 and CXCR2 chemokine receptors);
e) somatosin receptor, EP2/EP4, A2AR, CD39, CD73 receptor antagonists
f) targeted kinase inhibitors (such as CSF-1R, EGFR or VEGFR inhibitors);
g) angiogenesis inhibitors (such as anti-VEGF-A or Delta-like Ligand-4);
h) immune stimulating peptides or chemokines (such as CXCL9 or CXCL10);
i) cytokines (such as IL-1, IL-2, IL-3, IL-4, IL-5, IL-6, IL-7, IL-8, IL-9, IL-10, IL-11, IL_12, IL-13, IL_14, IL-15, IL-16, IL-17, IL-18, IL-19, IL-20 and IL-21, in particular IL-2, IL-12, IL-15, IL-21) and interferons (such as IFN-α, IFN-β and IFN-γ, in particular IFN-γ));
j) bispecific T-cell engagers (BiTEs) having at least one specificity against CD3 (e.g. CD3/CD19 BiTE) and NK cell engagers (e.g. CD16);
k) other bi-specific molecules (for example IL-2-containing or IL-15-containing molecules targeted towards tumour associated antigens, for example Epidermal growth factor receptors such as EGFR, Her-2, New York Esophageal Cancer-1 (NY-ESO-1), GD2, EpCAM or Melanoma Associated Antigen-3 (MAGE-A3));
l) oncolytic viruses (such as HSV virus (optionally which secretes GMCSF), Newcastle disease virus, Vaccinia virus, Adenovirus, NCD virus (Paramyxovirus), Sindbis virus, Respiratory syncytial virus, Measles virus, Cocksackie virus, Vesicular stomatitis virus, Mengo virus, Reovirus, Parvovirus, Maraba virus (rhabdovirus) and Echnvirus);
m) vaccination with tumour associated antigens (such as New York Esophageal Cancer-1 [NY-ESO-1], Melanoma Associated Antigen-3 [MAGE-3], BCMA);
n) cell-based therapies (such as chimeric Antigen Receptor-T cells (CAR-T) for example expressing anti-CD19, Chimeric Antigen Receptor natural killer cells (CAR-NK), anti-EpCam, anti-VEGF/VEGFR or anti-mesothelin);
o) adoptive transfer of tumour specific T-cells (e.g. autologous Tumour-Infiltrating Lymphocytes (TILs)) or LAK cells; and
p) tumour associated antigen antibodies (such as anti-CEA, anti-GD2, anti-glypican-3, anti-glypican-2, anti-nectin-4 and anti-mesothelin);
and/or optionally wherein the further therapy is selected from chemotherapy, radiotherapy and/or surgical removal of tumours.

In one embodiment, the chemokine receptor antagonist is an antagonist of the adenosine pathway (e.g. antibody that targets the adenosine pathway). In another embodiment, the further therapeutic agent is selected from anti-CD47, anti-SIRPα, anti-KIR, anti-CD70 and anti-GARP antibodies. In another embodiment, the further therapeutic agent is selected from anti-HER2, anti-CD20, anti-NKG2A, anti-TRAIL and anti-Notch antibodies. In another embodiment, the further therapeutic agent is selected from anti-PVRIG, anti-ILDR2 and anti-CD55 antibodies. In one embodiment, the further therapeutic agent is in combination with a further therapy selected from radiotherapy, chemotherapy and/or antibody drug conjugates (ADCs). In another embodiment, the further therapeutic agent is a small molecule selected from IDO inhibitors, PARP inhibitors, BTK inhibitors, MAPK inhibitors, STING agonists, TLR agonists (e.g. TLR7/9, TLR8), A2aR antagonists, CXCR2 antagonists, STAT3 transcription factor inhibitors, EGFR inhibitors, BRAF inhibitors, Wee1 tyrosine kinase inhibitors, FGFR-1/3 inhibitors and/or PDGFR inhibitors. In one embodiment, the further therapeutic agent is selected from ImmTacs, antisense/siRNA/shRNA, ProTACs and prodrug conjugates.

In one embodiment, there is provided a method or use as described herein which comprises administering to the patient (e.g. human) an anti-CXCR4 antibody or antigen-binding fragment as described herein in addition to chemotherapy or radiotherapy. In one embodiment, there is provided a method or use as described herein which comprises administering to the patient an anti-CXCR4 antibody or antigen-binding fragment as described herein, prior to or after administering chemotherapy and/or radiotherapy, wherein the antibody or antigen-binding fragment increases the efficacy of the chemotherapy and/or radiotherapy (as compared to when no antibody or antigen-binding fragment is administered). Administering anti-CXCR4 antibodies or antigen-binding fragments as described herein after administrating chemotherapy and/or radiotherapy may be beneficial as cancer cells may be more sensitive to treatment.

In a preferred embodiment, the further therapeutic agent is a PD-1/PD-L1 signalling inhibitor (e.g. an anti-PD-1 antibody or an anti-PD-L1 antibody). In one embodiment, the further therapy is a combination of a checkpoint inhibitor (as described above) and chemotherapy. In one embodiment, the further therapeutic agent is a combination of two checkpoint inhibitors (as described above).

In one embodiment, there is provided a further therapeutic agent which comprises an antibody or antigen-binding fragment which comprises an antigen-binding site that specifically binds an antigen as detailed below.

In one embodiment, the further therapeutic agent is an anti-PD-L1 antibody or antigen-binding fragment thereof which specifically binds PD-L1, e.g. hPD-L1. In one embodiment, the anti-PD-L1 antibody or antigen-binding fragment thereof comprises any one of the anti-PD-L1 antibodies selected from atezolizumab/MPDL3280A (Roche), avelumab/MSB0010718C (Merck), BMS-936559/MDX-1105 (BMS), durvalumab/Medi4736 (Medimmune), KN-035, CA-170, FAZ-053 M7824, ABBV-368, LY-3300054, GNS-1480, YW243.55.S70, REGN3504 and any of the PD-L1 antibodies described in WO2019/129136, WO2019/129211, WO2019/132533, WO2019/072869, WO2019/075097, WO2019/085238, WO2019/040780, WO2019/005634, WO2019/005635, WO2019/005636, WO2019/005637, WO2019/005638, WO2019/005639, WO2019/005642, WO2019/005641, WO2019/005640, WO2018/222949, WO2018/194496, WO2018/195226, WO2018/162430, WO2018/162446, WO2018/162749, WO2018/181064, WO2018/153320, WO2018/133873, WO2018/136553, WO2018/119475, WO2018/080812, WO2018/054940, WO2018/034225, WO2018/009894, WO2018/024237, WO2018/026249, WO2018/005682, WO2017/072280, WO2017/215590, WO2017/072273, WO2017/218435, WO2017/196867, WO2017/197667, WO2017/148424, WO2017/118321, WO2017/132562, WO2017/097407, WO2017/084495, WO2017/087547, WO2017/091429, WO2017/034916, WO2017/020291, WO2017/020858, WO2017/020802, WO2017/020801, WO2016/111645, WO2016/050721, WO2016/197367, WO2016/061142, WO2016/149201, WO2016/000619, WO2016/160792, WO2016/022630, WO2016/007235, WO2015/036499, WO2015/179654, WO2015/173267, WO2015/181342, WO2015/109124, WO2015/195163, WO2015/112805, WO2015/061668, WO2014/159562, WO2014/165082, WO2014/100079, WO2014/055897, WO2013/181634, WO2013/173223, WO2013/079174, WO2012/145493, WO2011/066389, WO2010/077634, WO2010/036959, WO2010/089411 or WO2007/005874, which antibodies are incorporated herein by reference.

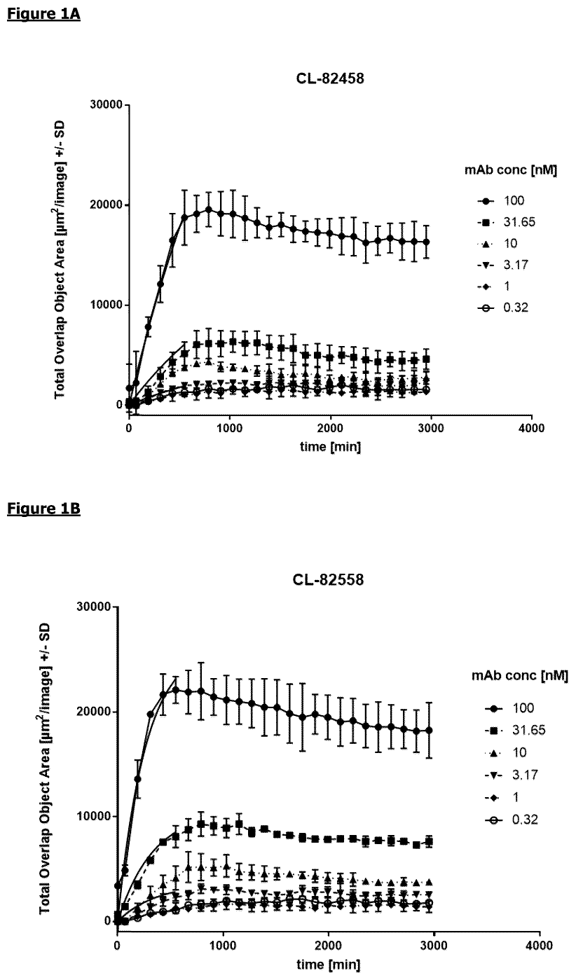

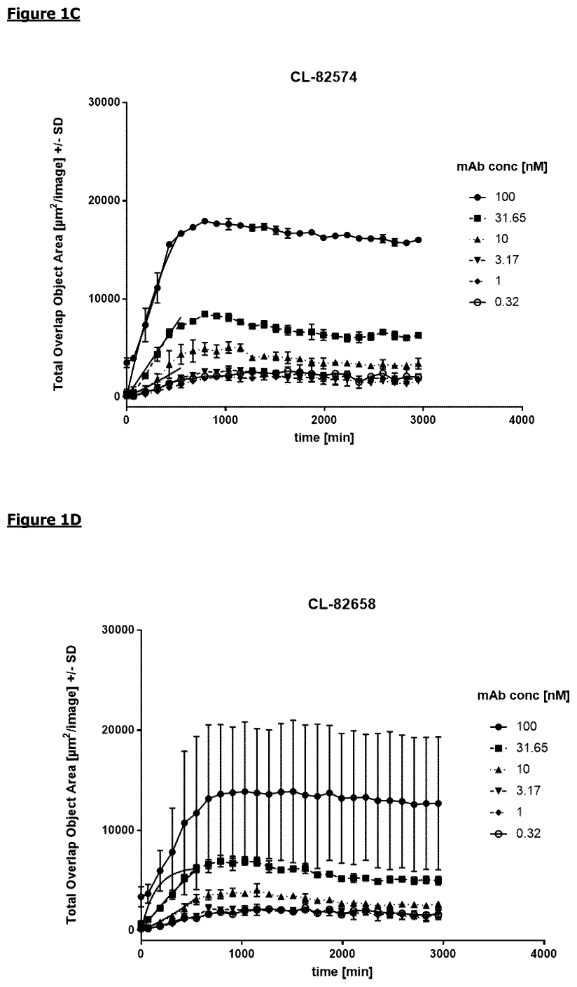

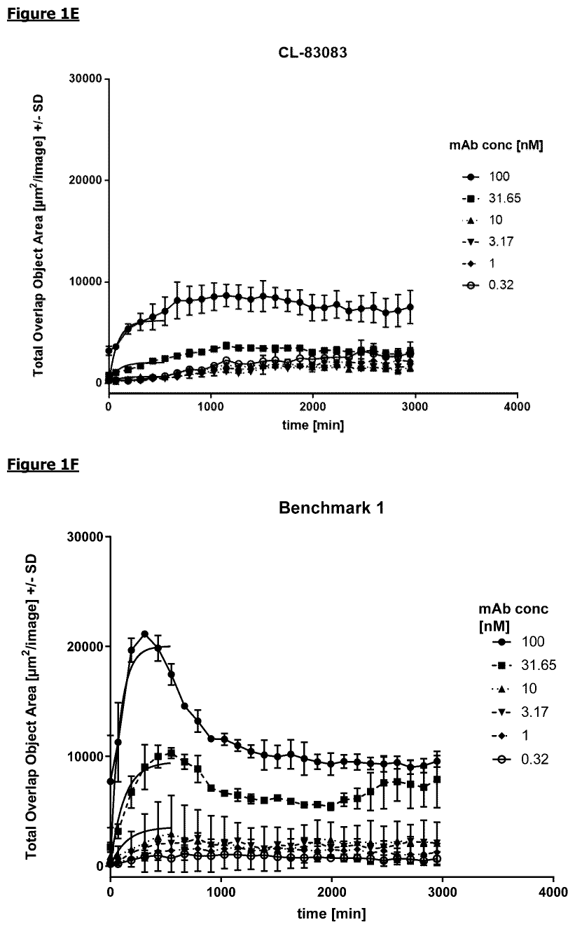

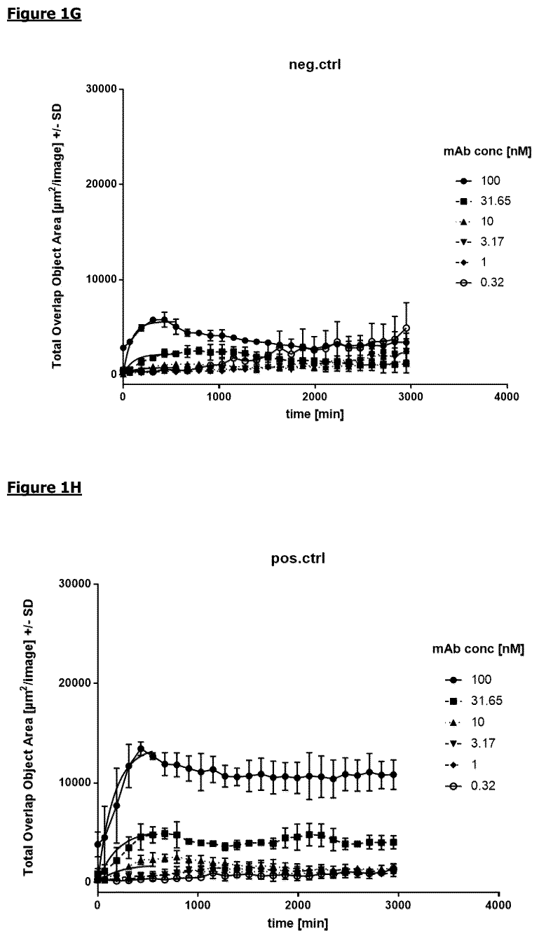

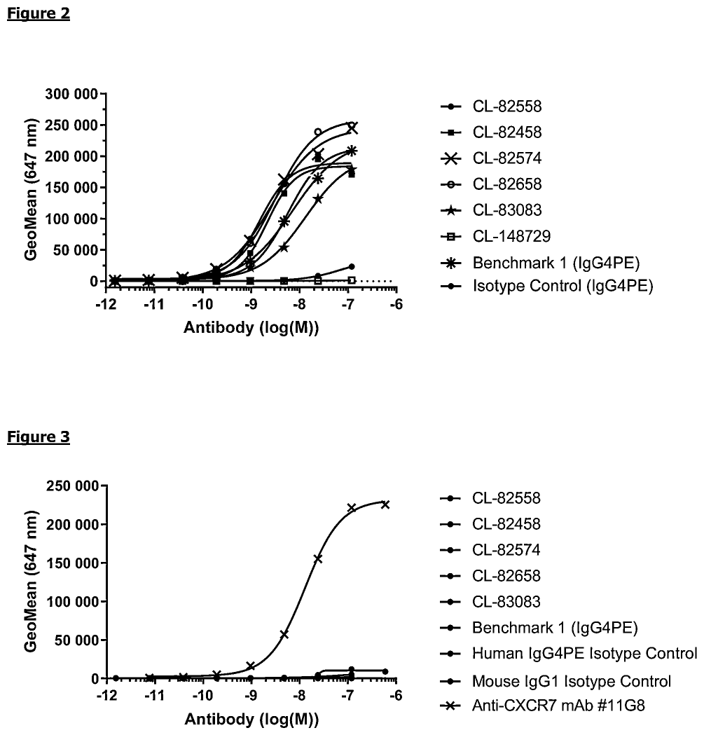

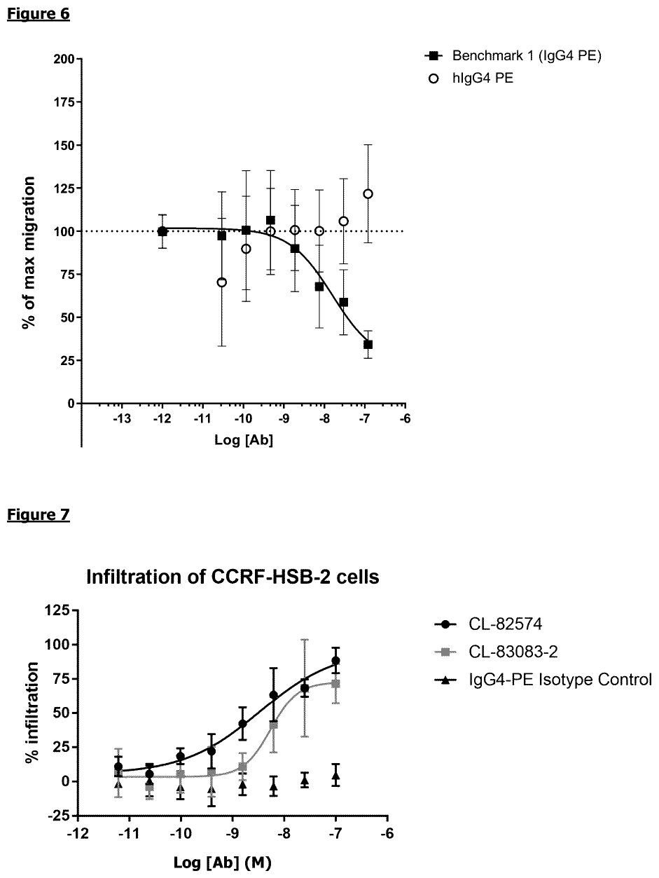

In one embodiment, the further therapeutic agent is an anti-PD-1 antibody or antigen-binding fragment thereof which specifically binds PD-1, e.g. hPD-1. In one embodiment, the anti-PD-1 antibody or antigen-binding fragment thereof comprises any one of the anti-PD-1 antibodies selected from pembrolizumab (Keytruda™/MK-3475), nivolumab (Opdivo™/BMS-936558/MDX-1106), MEDI-0680/AMP514, PDR001, Lambrolizumab, BMS-936558, REGN2810, BGB-A317, BGB-108, PDR-001, SHR-1210, JS-001, JNJ-63723283, AGEN-2034, PF-06801591, genolimzumab, MGA-012, IBI-308, BCD-100, TSR-042 ANA011, AUNP-12, KD033, MCLA-134, mDX400, muDX400, STI-A1110, AB011, 244C8, 388D4, XCE853, or pidilizumab/CT-011, or from any one of the anti-PD-1 antibodies described in WO2015/112800 & US2015/0203579 (including the antibodies in Tables 1 to 3), U.S. Pat. Nos. 9,394,365, 5,897,862 and 7,488,802, WO2017/087599 (including antibody SSI-361 and SHB-617), WO2017/079112, WO2017/071625 (including deposit C2015132, hybridoma LT004, and antibodies 6F5/6 F5 (Re), 6F5H1 L1 and 6F5 H2L2), WO2017/058859 (including PD1AB-1 to PD1AB-6), WO2017/058115 (including 67D9, c67D9, and hu67D9), WO2017/055547 (including 12819.15384, 12748.15381, 12748.16124, 12865.15377, 12892.15378, 12796.15376, 12777.15382, 12760.15375 and 13112.15380), WO2017/040790 (including AGEN2033w, AGEN2034w, AGEN2046w, AGEN2047w, AGEN2001w and AGEN2002w), WO2017/025051 & WO2017/024515 (including 1.7.3 hAb, 1.49.9 hAb, 1.103.11 hAb, 1.103.11-v2 hAb, 1.139.15 hAb and 1.153.7 hAb), WO2017/025016 & WO2017/024465 (including antibody A to antibody I), WO2017/020858 & WO2017/020291 (including 1.4.1, 1.14.4, 1.20.15 and 1.46.11), WO2017/019896 & WO2015/112900 & US2015/0210769 (including BAP049-hum01 to BAP049-hum16 and BAP049-Clone-A to BAP049-Clone-E), WO2017/019846 (including PD-1 mAb 1 to PD-1 mAb 15), WO2017/016497 (including MHC723, MHC724, MHC725, MHC728, MHC729, m136-M13, m136-M19, m245-M3, m245-M5 and m136-M14), WO2016/201051 (including antibody EH12.2H7, antibody hPD-1 mAb2, antibody hPD-1 mAb7, antibody hPD-1 mAb9, antibody hPD-1 mAb15, or an anti-PD-1 antibody selected from Table 1), WO2016/197497 (including DFPD1-1 to DFPD1-13), WO2016/197367 (including 2.74.15 and 2.74.15.hAb4 to 2.74.15.hAb8), WO2016/196173 (including the antibodies in Table 5, and FIGS. 1-5), WO2016/127179 (including R3A1, R3A2, R4B3, and R3D6), WO2016/077397 (including the antibodies described in Table 1 of Example 9), WO2016/106159 (including the murine antibodies in Table 3 of Example 2 and the humanised antibodies in Tables 7, 8 and 9 of Example 3), WO2016/092419 (including C1, C2, C3, EH12.1, mAb7-G4, mAb15-G4, mAb-AAA, mAb15-AAA), WO2016/068801 (including clone A3 and its variants and the other antibodies described in FIGS. 1 to 4), WO2016/014688 (including 10D1, 4C10, 7D3, 13F1, 15H5, 14A6, 22A5, 6E1, 5A8, 7A4, and 7A4D and the humanised antibodies of Examples 9/10), WO2016/015685 (including 10F8, BA08-1, BA-08-2 and 15H6), WO2015/091911 &WO2015/091910 (including the anti-canine PD-1 antibodies in Examples 2, 3 and 4), WO2015/091914 (including the anti-canine PD-1 antibodies in Table 3), WO2015/085847 (including mAb005, H005-1 to H005-4), WO2015/058573 (including cAB7), WO2015/036394 (including LOPD180), WO2015/035606 (including the antibodies in Table 1 of Example 2, in Tables 14, 15 and 16 of Example 7 and in tables 20, 21 and 22 of Example 11), WO2014/194302 (including GA2, RG1B3, RG1H10, RG2A7, RG2H10, SH-A4, RG4A6, GA1, GB1, GB6, GH1, A2, C7, H7, SH-A4, SH-A9, RG1H11, and RG6B), WO2014/179664 (including 9A2, 10B11, 6E9, APE1922, APE1923, APE1924, APE1950, APE1963 and APE2058), WO2014/206107 (including clone 1, 10, 11, 55, 64, 38, 39, 41 and 48), WO2012/135408 (including h409A11, h409A16, and h409A17), WO2012/145493 (including antibodies 1E3, 1E8, 1H3 and h1H3 Var 1 to h1H3 Var 14), WO2011/110621 (including antibody 949 and the modified versions disclosed in FIGS. 1 to 11), WO2011/110604 (including antibody 948 and the modified versions disclosed in FIGS. 3 to 11), WO2010/089411 (including CNCM deposit number 1-4122, 1-4080 or 1-4081), WO2010/036959 (including the antibodies in Table 1 of Example 1), WO2010/029435 & WO2010/029434 (including clones 2, 10 and 19), WO2008/156712 (including hPD-1.08A, hPD-1.09A, h409A11, h409A16 and h409A17 and the antibodies described in Example 2, Table H, Example 4 and table IV), WO2006/121168 (including clones 17D8, 4H1, 5C4, 4A11, 7D3, 5F4, and 2D3), WO2004/004771 or WO2004/056875 (including PD1-17, PD1-28, PD1-33, PD1-35, PD1-F2 and the Abs described in Table 1); the sequences and features of the anti-PD-1 antibodies are incorporated herein by reference.

In one embodiment, the anti-CXCR4 antibody or antigen-binding fragment described herein is used in combination with a PD-1 or PD-L1 signalling inhibitor (such as an anti-PD-1 antibody or antigen-binding fragment or an anti-PD-L1 antibody or antigen-binding fragment) for use in treating or preventing a CXCR4-mediated disease or condition (as described herein) wherein one, more or all of a to i apply:
a. the sensitivity of the cancer cells to the host immune responses is increased;
b. immune suppression in the tumour is reduced;
c. the T-cells cause apoptosis in the tumour
d. cancer cell recognition is increased within the tumour;
e. cancer cell growth is inhibited;
f. cancer cells are eliminated;
g. tumour mass is reduced;
h. the tumour comprises FAP+ stromal cells; or
i. the tumour is resistant to immunotherapy.

Still further embodiments include methods of treating a proliferative or invasion-related disease in a mammal by administering to the animal a therapeutically effective dose of an anti-CXCR4 antibody or antigen-binding fragment as described herein. Without being bound by theory, this may be achieved by inhibiting CXCL12 driven T-cell exclusion from the tumour and/or stroma or by inhibiting tumour suppressor cells (e.g. Myelin-derived suppressor cells or T-regs) from the tumour and/or stroma. Thus, in one embodiment, any of the anti-CXCR4 antibodies or antigen-binding fragments described herein reduce metastatic invasion and adhesion. In another embodiment, the anti-CXCR4 antibodies or antigen-binding fragments described herein reduce tumour angiogenesis. In another embodiment, the anti-CXCR4 antibodies or antigen-binding fragments described herein reduce tumour cell proliferation and chemoresistance.

### Detection

In another embodiment, the antibodies or antigen-binding fragments can be used to detect the presence, absence and/or level of CXCR4 expression in a sample. CXCR4 expression can be detected in vivo and/or in vitro and is useful in helping diagnose diseases or conditions that involve expression and/or overexpression of CXCR4. In another embodiment, anti-CXCR4 antibodies or antigen-binding fragments described herein are used in detection of diseases or conditions e.g. imaging (i.e. PET).

### Guide Patient Selection

In one embodiment, detection of CXCR4 expression using any of the anti-CXCR4 antibodies or antigen-binding fragments described herein can be used to guide patient selection. In one embodiment, the anti-CXCR4 antibodies or antigen-binding fragments thereof described herein can be used to assist in patient selection for therapeutic antibody treatment with an anti-CXCR4 antibody or antigen-binding fragment. In some cases, higher levels of CXCR4 may be indicative of successful therapy, whereas lower levels may indicate a reduced likelihood of success. Preferential expression of splice variants and/or protein processing may produce unique protein mixture profiles which may impact a patient's response to treatment or may change following treatment. These profiles may help to identify patients and define patient subsets who should receive treatment, continue to receive treatment or who should receive an alternative treatment. In another embodiment, the antibodies or antigen-binding fragments thereof can be used for detection of CXCR4 isoforms. Patient samples can include, for example, blood, plasma, serum, sputum, saliva, urine, CSF, tears, exhaled exogenous particle samples, cell supernatant, cell or tissue lysate or tissue samples.

### 4. Pharmaceutical Compositions

In one embodiment, there is provided a pharmaceutical composition comprising an effective amount of an anti-CXCR4 antibody or antigen-binding fragment as described herein and a pharmaceutically acceptable excipient, diluent or carrier. An effective amount of anti-CXCR4 antibody or antigen-binding fragment to be employed therapeutically will depend, for example, upon the therapeutic objectives, the route of administration, and the condition of the patient. In one embodiment, the composition includes other excipients or stabilizers.

Pharmaceutically acceptable carriers are known and include carriers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as Ethylenediaminetetraacetic acid (EDTA); sugar alcohols such as mannitol or sorbitol; salt-forming counterions such as sodium; and/or non-ionic surfactants such as TWEEN™, polyethylene glycol (PEG), and PLURONICS™.

The anti-CXCR4 antibodies or antigen-binding fragments as described herein or pharmaceutical compositions as described herein may be administered intravenously or through the nose, lung, for example, as a liquid or powder aerosol (lyophilized). The antibodies, antigen-binding fragments or compositions may also be administered parenterally or subcutaneously. When administered systemically, the composition should be sterile, pyrogen-free and in a physiologically acceptable solution having due regard for pH, isotonicity and stability. These conditions are known to those skilled in the art.

Methods of administering a prophylactic or therapeutic agent (e.g., an anti-CXCR4 antibody or antigen-binding fragment as described herein), or pharmaceutical composition include, but are not limited to, parenteral administration (e.g., intradermal, intramuscular, intraperitoneal, intravenous and subcutaneous), epidural, and mucosal (e.g., intranasal and oral routes). In a specific embodiment, a prophylactic or therapeutic agent (e.g., an anti-CXCR4 antibody or antigen-binding fragment as described herein), or a pharmaceutical composition is administered intranasally, intramuscularly, intravenously, or subcutaneously, in particular intravenously. The prophylactic or therapeutic agents, or compositions may be administered by any convenient route, for example by infusion or bolus injection, by absorption through epithelial or mucocutaneous linings (e.g., oral mucosa, intranasal mucosa, rectal and intestinal mucosa, etc.) and may be administered together with other therapeutic or biologically active agents. Administration can be systemic or local. Each dose may or may not be administered by an identical route of administration. In one embodiment, an anti-CXCR4 antibody or antigen-binding fragment thereof as described herein may be administered via multiple routes of administration simultaneously or subsequently to other doses of the same or a different anti-CXCR4 antibody or antigen-binding fragment thereof as described herein.

Various delivery systems are known and can be used to administer a prophylactic or therapeutic agent (e.g., an anti-CXCR4 antibody or antigen-binding fragment thereof as described herein), including, but not limited to, encapsulation in liposomes, microparticles, microcapsules, recombinant cells capable of expressing the antibody or antigen-binding fragment, receptor-mediated endocytosis (see, e.g., Wu and Wu, J. Biol. Chem. 262:4429-4432 (1987)), construction of a nucleic acid as part of a retroviral or other vector, etc. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO92/19244, WO97/32572, WO97/44013, WO98/31346, and WO99/66903, each of which is incorporated herein by reference their entirety.

In a specific embodiment, it may be desirable to administer a prophylactic or therapeutic agent, or a pharmaceutical composition as described herein locally to the area in need of treatment. This may be achieved by, for example, local infusion, by topical administration (e.g., by intranasal spray), by injection, or by means of an implant, said implant being of a porous, non-porous, or gelatinous material, including membranes, such as sialastic membranes, or fibres. When administering a prophylactic or therapeutic agent (e.g., an anti-CXCR4 antibody or antigen-binding fragment as described herein), care must be taken to use materials to which the antibody or antigen-binding fragment does not absorb.

In one embodiment, there is provided a pharmaceutical composition comprising an anti-CXCR4 antibody or antigen-binding fragment as described herein and a pharmaceutically acceptable excipient, diluent or carrier and further comprising one or more (e.g. more than one, or for example two) further therapeutic agents independently selected from the group consisting of:
a) immune checkpoint inhibitors (such as anti-PD-1 antibodies, anti-PD-L1 antibodies, anti-TIM-3 antibodies, anti-CTLA-4 antibodies, anti-TIGIT antibodies, anti-BTLA antibodies, anti-VISTA antibodies and anti-LAG-3 antibodies);
b) immune stimulators (such as anti-OX40 antibodies, anti-HVEM antibodies, anti-CD27 antibodies, anti-CD28 antibodies, anti-CD137L antibodies, anti-OX40L antibodies, anti-GITRL antibodies, anti-ICOSL antibodies, anti-PD-L2 antibodies, anti-GITR antibodies, anti-CD137 antibodies, anti-ICOS antibodies and anti-CD40 antibodies);
c) anti-CSF1R antibodies, anti-CCR4 antibodies, anti-CD39 antibodies, anti-CD73 antibodies, anti-CD96 antibodies, anti-CXCR2 antibodies, anti-CD200 antibodies, anti-GARP antibodies, anti-SIRPα antibodies, anti-CXCL9 antibodies, anti-CXCL10 antibodies, anti-CXCL11 antibodies and anti-CD155 antibodies;
d) chemokine receptor antagonists (such as CCR4, and CXCR2 chemokine receptors);
e) somatosin receptor, EP2/EP4, A2AR, CD39, CD73 antagonists;
f) targeted kinase inhibitors (such as CSF-1R, EGFR or VEGFR inhibitors);
g) angiogenesis inhibitors (such as anti-VEGF-A or Delta-like Ligand-4);
h) immune stimulating peptides or chemokines (such as CXCL9 or CXCL10);
i) cytokines (such as IL-1, IL-2, IL-3, IL-4, IL-5, IL-6, IL-7, IL-8, IL-9, IL-10, IL-11, IL_12, IL-13, IL_14, IL-15, IL-16, IL-17, IL-18, IL-19, IL-20 and IL-21, in particular IL-2, IL-12, IL-15, IL-21 and interferons (such as IFN-α, IFN-β and IFN-γ, in particular IFN-γ));
j) bispecific T-cell engagers (BiTEs) having at least one specificity against CD3 (e.g. CD3/CD19 BiTE) and NK cell engagers (e.g. CD16);
k) other bi-specific molecules (for example IL-2-containing or IL-15-containing molecules targeted towards tumour associated antigens, for example Epidermal growth factor receptors such as EGFR, Her-2, New York Esophageal Cancer-1 (NY-ESO-1), GD2, EpCAM or Melanoma Associated Antigen-3 (MAGE-A3));
l) oncolytic viruses (such as HSV virus (optionally which secretes GMCSF), Newcastle disease virus, Vaccinia virus, Adenovirus, NCD virus (Paramyxovirus), Sindbis virus, Respiratory syncytial virus, Measles virus, Cocksackie virus, Vesicular stomatitis virus, Mengo virus, Reovirus, Parvovirus, Maraba virus (rhabdovirus) and Echnvirus);
m) vaccination with tumour associated antigens (such as New York Esophageal Cancer-1 [NY-ESO-1], Melanoma Associated Antigen-3 [MAGE-3], BCMA);
n) cell-based therapies (such as chimeric Antigen Receptor-T cells (CAR-T) for example expressing anti-CD19, Chimeric Antigen Receptor natural killer cells (CAR-NK), anti-EpCam, anti-VEGF/VEGFR or anti-mesothelin);
o) adoptive transfer of tumour specific T-cells or LAK cells;
p) tumour associated antigen antibodies (such as anti-CEA, anti-GD2, anti-glypican-3, anti-glypican-2, anti-nectin-4 and anti-mesothelin); and
q) chemotherapy.

In one embodiment, there is provided a pharmaceutical composition as described herein, or a kit comprising a pharmaceutical composition as described herein, wherein the composition is for therapy; or is for treating and/or preventing a CXCR4-mediated disease or condition as described hereinabove.

In one embodiment, there is provided a pharmaceutical composition, as described herein, in combination with, or kit comprising, a label or instructions for use to treat and/or prevent said CXCR4-mediated disease or condition, as described herein, in a patient; optionally wherein the label or instructions comprise a marketing authorisation number (e.g., an FDA or EMA authorisation number). Optionally the label or instructions can be a notice in the form prescribed by a governmental agency regulating the manufacture, use or sale of pharmaceuticals or biological products, which notice reflects approval by the agency of manufacture, use or sale for human administration, e.g., an authorisation number. In one embodiment, the kit includes an anti-CXCR4 antibody or antigen-binding fragment described herein and instructions to administer the antibody or antigen-binding fragment to a patient in need of treatment. In one embodiment, the kit comprises an IV or injection device that comprises said anti-CXCR4 antibody or antigen-binding fragment as described herein. In one embodiment, the antibody or antigen-binding fragment is administered intravenously. In one embodiment, the antibody or antigen-binding fragment is administered subcutaneously. In an example, the anti-CXCR4 antibody or antigen-binding fragment as described herein is contained in a medical container, e.g., a vial, syringe, IV container or an injection device (such as an intraocular or intravitreal injection device). In an example, the anti-CXCR4 antibody or antigen-binding fragment is in vitro, for example, in a sterile container. In one embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilising agent and a local anaesthetic such as lignocaine to ease pain at the site of the injection. Such compositions, however, may be administered by a route other than intravenous. Generally, the ingredients of compositions are supplied either separately or mixed together in unit dosage form, for example, as a dry lyophilized powder or water free concentrate in a hermetically sealed container such as an ampoule or sachets indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. The further therapeutic agents of this disclosure may be delivered by any method, which methods are well-known to those skilled in the art. For example, the further therapeutic agents may be delivered orally, systemically or locally (to the tumour environment). In one embodiment, the further therapeutic agent is delivered orally. In one embodiment, the further therapeutic agent is delivered systemically (e.g. intravenously). In one embodiment, the further therapeutic agent is delivered locally to the tumour environment. In another embodiment, the anti-CXCR4 antibody and/or further therapeutic agent is locally delivered to a site after surgical removal of all or (a substantial) part of the tumour.

### 5. Kits and Articles of Manufacture

In another embodiment, an article of manufacture that includes a container in which a composition containing an anti-CXCR4 antibody or antigen-binding fragment described herein and a package insert or label indicating that the composition can be used to treat diseases or conditions characterized by the expression or overexpression of CXCR4 is provided. In one embodiment, there is provided a kit for treating and/or preventing a CXCR4-mediated disease or condition, the kit comprising an anti-CXCR4 antibody or antigen-binding fragment as described herein in any embodiment or combination of embodiments (and optionally a further therapeutic agent as described elsewhere herein) optionally in combination with a label or instructions for use to treat and/or prevent said disease or condition in a human; optionally wherein the label or instructions comprise a marketing authorisation number (e.g., an FDA or EMA authorisation number); optionally wherein the kit comprises an IV or injection device that comprises the antibody or antigen-binding fragment. In another embodiment, the kit comprises an antibody or antigen-binding fragment contained within a container or an IV bag. In another embodiment, the container or IV bag is a sterile container or a sterile IV bag. In another embodiment, the antibody or antigen-binding fragments formulated into a pharmaceutical composition contained within a (sterile) container or contained within a (sterile) IV bag. In a further embodiment, the kit further comprises instructions for use.

### 6. Additional Embodiments

In one embodiment, there is provided an antibody or antigen-binding fragment thereof which specifically binds to CXCR4, wherein the antibody or antigen-binding fragment enables CD8⁺ T-cells to infiltrate a tumour.

In one embodiment, there is provided an antibody or antigen-binding fragment thereof, which specifically binds to CXCR4, and comprises a V_{H} domain comprising a CDRH3, wherein the CDRH3 sequence is identical to the CDRH3 sequence of an antibody or antigen-binding fragment as described herein, or comprises 3, 2 or 1 amino acid substitution(s).

In one embodiment, there is provided an antibody or antigen-binding fragment thereof, which specifically binds to CXCR4, and comprises a V_{H} domain comprising a CDRH1, a CDRH2 and a CDRH3, wherein the CDRH1-3 sequences are identical to the CDRH1-3 sequences of an antibody or antigen-binding fragment as described herein, or comprise 3, 2 or 1 amino acid substitution(s).

In one embodiment, there is provided an antibody or antigen-binding fragment thereof, which specifically binds to CXCR4, and comprises a V_{H} domain wherein the V_{H} domain comprises an amino acid sequence which is identical to or at least 90% identical to a V_{H} amino acid sequence of an antibody or antigen-binding fragment as described herein.

In one embodiment, there is provided an antibody or antigen-binding fragment thereof, which specifically binds to CXCR4, and comprises a V_{L} domain comprising a CDRL3, wherein the CDRL3 sequence is identical to the CDRL3 sequence of an antibody or antigen-binding fragment as described herein, or comprises 3, 2 or 1 amino acid substitution(s).

In one embodiment, there is provided an antibody or antigen-binding fragment thereof, which specifically binds to CXCR4, and comprises a V_{L} domain comprising a CDRL1, a CDRL2 and a CDRL3, wherein the CDRL1-3 sequences are identical to the CDRL1-3 sequences of an antibody or antigen-binding fragment as described herein, or comprise 3, 2 or 1 amino acid substitution(s).

In one embodiment, there is provided an antibody or antigen-binding fragment thereof, which specifically binds to CXCR4, and comprises a V_{L} domain wherein the V_{L} domain comprises an amino acid sequence which is identical to or at least 90% identical to a V_{L} amino acid sequence of an antibody or antigen-binding fragment as described herein.

In one embodiment, there is provided an antibody or antigen-binding fragment thereof, which specifically binds to CXCR4, and comprises a V_{H} domain and a V_{L} domain, wherein the V_{H} domain comprises an amino acid sequence which is identical or at least 90% identical to a V_{H} amino acid sequence of an antibody or antigen-binding fragment as described herein; and the V_{L} domain comprises an amino acid sequence which is identical or at least 90% identical to V_{L} amino acid sequence of an antibody or antigen-binding fragment as described herein.

In one embodiment, there is provided an antibody or antigen-binding fragment thereof, which specifically binds to CXCR4, and comprises a heavy chain and a light chain, wherein the heavy chain amino acid sequence is identical or at least 90% identical to the heavy chain amino acid sequence of an antibody or antigen-binding fragment as described herein, and the light chain amino acid sequence is identical or at least 90% identical to the light chain amino acid sequence of an antibody or antigen-binding fragment as described herein.

In one embodiment, there is provided an antibody or antigen-binding fragment thereof, which specifically binds to CXCR4, and competes with an antibody or antigen-binding fragment as described herein for binding to CXCR4.

In one embodiment, there is provided an antibody or antigen-binding fragment thereof, which specifically binds to CXCR4, wherein the antibody or antigen-binding fragment binds to an epitope that is identical to an epitope to which an antibody or antigen-binding fragment as described herein specifically binds.

In one embodiment, there is provided an antibody or antigen-binding fragment thereof, which specifically binds to CXCR4, wherein the antibody or fragment does not induce apoptosis in T-cells (optionally CD8⁺ T-cells).

In one embodiment, there is provided an antibody or antigen-binding fragment thereof, which specifically binds to CXCR4, wherein the antibody or fragment binds to cynomolgus CXCR4 with an EC₅₀ of from 0.2 to 4 nM or less.

In one embodiment, there is provided an antibody or antigen-binding fragment thereof, which specifically binds to CXCR4, wherein the antibody or antigen-binding fragment does not bind to CXCR7.

In one embodiment, there is provided an antibody or antigen-binding fragment thereof as described herein for use in treating a CXCR4-mediated disease or condition as described herein.

In one embodiment, there is provided a use of an antibody or antigen-binding fragment as described herein, in the manufacture of a medicament for treating a CXCR4-mediated disease or condition as described herein.

In one embodiment, there is provided a method of treating a CXCR4-mediated disease or condition as described herein in a patient, comprising administering to said patient (e.g. human) a therapeutically effective amount of an antibody or antigen-binding fragment as described herein, wherein the CXCR4-mediated disease or condition is thereby treated.

In one embodiment, there is provided a kit comprising a pharmaceutical composition comprising an anti-CXCR4 antibody or antigen-binding fragment as described herein, wherein the composition is for treating a CXCR4-mediated disease or condition as described herein.

In one embodiment, there is provided a nucleic acid that encodes an anti-CXCR4 antibody or antigen-binding fragment or any part thereof disclosed herein.

In one embodiment, there is provided a vector comprising a nucleic acid that encodes an anti-CXCR4 antibody or antigen-binding fragment or any part thereof disclosed herein.

In one embodiment, there is provided a host cell comprising a nucleic acid that encodes an anti-CXCR4 antibody or antigen-binding fragment or any part thereof disclosed herein, or a vector as described in herein.

7. Informal Sequence Listing

| SEQ | | | De- | |
|---|---|---|---|---|
| ID | | | scrip- | |
| NO: | Name | | tion | Sequence |
| 1 | Human | | Amino | MEGISIYTSDNYTEEMGSGDYDSMKEPCFREENANFNKIFLPTIYSIIFLTGIVGNGLVILVMGYQK |
| | CXCR4 | | acid | KLRSMTDKYRLHLSVADLLFVITLPFWAVDAVANWYFGNFLCKAVHVIYTVNLYSSVLILAFISLDR |
| | | | se- | YLAIVHATNSQRPRKLLAEKVVYVGVWIPALLLTIPDFIFANVSEADDRYICDRFYPNDLWVVVFQ |
| | | | quence | FQHIMVGLILPGIVILSCYCIIISKLSHSKGHQKRKALKTTVILILAFFACWLPYYIGISIDSFILL |
| | | | of | EIIKQGCEFENTVHKWISITEALAFFHCCLNPILYAFLGAKFKTSAQHALTSVSRGSSLKILSKGKR |
| | | | human | GGHSSVSTESESSSFHSSGGGGSGGGGSHHHHHH |
| | | | CXCR4 | |
| | | | used | |
| | | | for | |
| | | | cell | |
| | | | line | |
| | | | gener- | |
| | | | ation | |
| 2 | Human | | Amino | MEGISIYTSDNYTEEMGSGDYDSMKEPCFREENANFNKIFLPTIYSIIFLTGIVGNGLVILVMGYQK |
| | CXCR4 | | acid | KLRSMTDKYRLHLSVADLLFVITLPFWAVDAVANWYFGNFLCKAVHVIYTVNLYSSVLILAFISLDR |
| | | | se- | YLAIVHATNSQRPRKLLAEKVVYVGVWIPALLLTIPDFIFANVSEADDRYICDRFYPNDLWVVVFQ |
| | | | quence | FQHIMVGLILPGIVILSCYCIIISKLSHSKGHQKRKALKTTVILILAFFACWLPYYIGISIDSFILL |
| | | | of | EIIKQGCEFENTVHKWISITEALAFFHCCLNPILYAFLGAKFKTSAQHALTSVSRGSSLKILSKGKR |
| | | | human | GGHSSVSTESESSSFHSS |
| | | | CXCR4. | |
| | | | Uni- | |
| | | | prot | |
| | | | ID: | |
| | | | P61073 | |
| 3 | Human | | Codon | atggaaggcatcagcatctacaccagcgacaactacaccgaggaaatgggcagcggcgactacgaca |
| | CXCR4 | | opti- | gcatgaaggaaccctgcttccgggaagagaacgccaacttcaacaagatcttcctgcccacaatcta |
| | | | mised | cagcatcatctttctgaccggcatcgtgggcaacggactcgtgatcctcgtgatgggctaccagaaa |
| | | | nu- | aagctgcggagcatgaccgacaagtaccggctgcacctgagcgtggccgacctgctgttcgtgatca |
| | | | cleic | ccctgcctttctgggccgtggacgccgtggccaattggtacttcggcaacttcctgtgcaaggccgt |
| | | | acid | gcacgtgatctacaccgtgaacctgtacagcagcgtgctgatcctggccttcatcagcctggacaga |
| | | | se- | tacctggccatcgtgcacgccaccaacagccagcggcctagaaagctgctggccgagaaggtggtgt |
| | | | quence | acgtgggcgtgtggattcccgccctgctgctgaccatccccgacttcatcttcgccaacgtgtccga |
| | | | of | ggccgacgaccggtacatctgcgaccggttctaccccaacgacctgtgggtggtggtgttccagttc |
| | | | human | cagcacatcatggtgggactgatcctgcctggcatcgtgattctgagctgctactgcatcatcatca |
| | | | CXCR4 | gcaagctgagccacagcaagggccaccagaagcggaaggccctgaaaaccaccgtgatcctgattct |
| | | | | ggctttcttcgcctgctggctgccctactacatcggcatctccatcgacagcttcatcctgctggaa |
| | | | | atcatcaagcagggctgcgagttcgagaacaccgtgcacaagtggatcagcattaccgaggccctgg |
| | | | | cctttttccactgctgcctgaaccctatcctgtacgccttcctgggcgccaagttcaagacctctgc |
| | | | | ccagcacgccctgaccagcgtgtccagaggaagcagcctgaagatcctgagcaagggcaagagaggc |
| | | | | ggccacagctccgtgtctacagagagcgagagcagcagcttccacagctctggcggcggaggatctg |
| | | | | ggggaggcggatctcaccaccatcaccatcac |
| 4 | Human | | Amino | MDLHLFDYSEPGNFSDISWPCNSSDCIVVDTVMCPNMPNKSVLLYTLSFIYIFIFVIGMIANSVVV |
| | CXCR7 | | acid | WVNIQAKTTGYDTHCYILNLAIADLWVVLTIPVWVVSLVQHNQWPMGELTCKVTHLIFSINLFGS |
| | | | se- | IFFLTCMSVDRYLSITYFTNTPSSRKKMVRRVVCILVWLLAFCVSLPDTYYLKTVTSASNNETYCRS |
| | | | quence | FYPEHSIKEWLIGMELVSVVLGFAVPFSIIAVFYFLLARAISASSDQEKHSSRKIIFSYVVVFLVCW |
| | | | of | LPYHVAVLLDIFSILHYIPFTCRLEHALFTALHVTQCLSLVHCCVNPVLYSFINRNYRYELMKAFIF |
| | | | human | KYSAKTGLTKLIDASRVSETEYSALEQSTK |
| | | | CXCR7 | |
| | | | used | |
| | | | for | |
| | | | cell | |
| | | | line | |
| | | | gener- | |
| | | | ation | |
| 5 | Human | | Amino | MDLHLFDYSEPGNFSDISWPCNSSDCIVVDTVMCPNMPNKSVLLYTLSFIYIFIFVIGMIANSVVV |
| | CXCR7 | | acid | WVNIQAKTTGYDTHCYILNLAIADLWVVLTIPVWVVSLVQHNQWPMGELTCKVTHLIFSINLFGS |
| | | | se- | IFFLTCMSVDRYLSITYFTNTPSSRKKMVRRVVCILVWLLAFCVSLPDTYYLKTVTSASNNETYCRS |
| | | | quence | FYPEHSIKEWLIGMELVSVVLGFAVPFSIIAVFYFLLARAISASSDQEKHSSRKIIFSYVVVFLVCW |
| | | | of | LPYHVAVLLDIFSILHYIPFTCRLEHALFTALHVTQCLSLVHCCVNPVLYSFINRNYRYELMKAFIF |
| | | | human | KYSAKTGLTKLIDASRVSETEYSALEQSTK |
| | | | CXCR7. | |
| | | | Uni- | |
| | | | prot | |
| | | | ID: | |
| | | | P25106 | |
| 6 | Human | | Nu- | atggacctgcacctgttcgactattccgagcctggcaacttctccgacatctcctggccttgcaact |
| | CXCR7 | | cleic | ccagcgactgcatcgtggtggacaccgtgatgtgccccaacatgcccaacaagtccgtgctgctgta |
| | | | acid | caccctgtccttcatctacatcttcatcttcgtgatcggcatgatcgccaactccgtggtcgtgtgg |
| | | | se- | gtcaacatccaggctaagaccaccggctacgacacccactgctacatcctgaacctggctatcgccg |
| | | | quence | acctgtgggtcgtgctgacaatccctgtgtgggttgtgtccctggtgcagcacaaccagtggcctat |
| | | | of | gggagagctgacctgcaaagtgacccacctgatcttctccatcaacctgttcggctctatcttcttc |
| | | | human | ctgacctgtatgtccgtggaccgctacctgtccatcacctacttcaccaacacaccctccagccgga |
| | | | CXCR7 | agaaaatggtccgacgggtcgtgtgcatcctcgtgtggctgctggccttctgtgtgtctctgcccga |
| | | | | cacctactacctgaaaaccgtgacctccgcctccaacaacgagacatactgccggtctttctacccc |
| | | | | gagcactccatcaaagaatggctgatcggaatggaactggtgtccgtggtgctgggcttcgccgtgc |
| | | | | ctttctctattatcgccgtgttctacttcctgctggccagagccatctccgcctcttccgatcaaga |
| | | | | gaagcactcctctcggaagatcatcttctcctacgtggtggtgtttctcgtgtgctggctgccttac |
| | | | | catgtggctgtgctgctggacatcttcagcatcctgcactacatccctttcacctgtcggctggaac |
| | | | | acgccctgtttaccgctctgcatgtgacccagtgcctgagcctggtgcactgctgtgtgaaccctgt |
| | | | | gctgtactccttcatcaaccggaactaccgctacgagctgatgaaggcctttatcttcaagtactcc |
| | | | | gccaagaccggcctgaccaagctgatcgacgcctccagagtgtccgagacagagtacagcgccctgg |
| 7 | Cyno- | | Amino | aacagtccaccaag |
| | molgus | | acid | KLRSMTDKYRLHLSVADLLFVITLPFWAVDAVANWYFGNFLCKAVHVIYTVNLYSSVLILAFISLDR |
| | CXCR4 | | se- | YLAIVHATNSQKPRKLLAEKVVYVGVWIPALLLTIPDFIFASVSEADDRYICDRFYPNDLWVVVFQF |
| | | | quence | QHIMVGLILPGIVILSCYCIIISKLSHSKGHQKRKALKTTVILILAFFACWLPYYIGISIDSFILLE |
| | | | of | IIKQGCEFENTVHKWISITEALAFFHCCLNPILYAFLGAKFKTSAQHALTSVSRGSSLKILSKGKRG |
| | | | cyno- | GHSSVSTESESSSFHSS |
| | | | molgus | |
| | | | CXCR4 | |
| | | | used | |
| | | | for | |
| | | | cell | |
| | | | line | |
| | | | gener- | |
| | | | ation | |
| 8 | Cyno- | | Amino | MEGISIYTSDNYTEEMGSGDYDSIKEPCFREENAHFNRIFLPTIYSIIFLTGIVGNGLVILVMGYQK |
| | molgus | | acid | KLRSMTDKYRLHLSVADLLFVITLPFWAVDAVANWYFGNFLCKAVHVIYTVNLYSSVLILAFISLDR |
| | CXCR4 | | se- | YLAIVHATNSQKPRKLLAEKVVYVGVWIPALLLTIPDFIFASVSEADDRYICDRFYPNDLWVVVFQF |
| | | | quence | QHIMVGLILPGIVILSCYCIIISKLSHSKGHQKRKALKTTVILILAFFACWLPYYIGISIDSFILLE |
| | | | of | GIIKQCEFENTVHKWISITEALAFFHCCLNPILYAFLGAKFKTSAQHALTSVSRGSSLKILSKGKRG |
| | | | cyno- | GHSSVSTESESSSFHSS |
| | | | molgus | |
| | | | CXCR4. | |
| | | | Uni- | |
| | | | prot | |
| | | | ID: | |
| | | | Q28474 | |
| 9 | Cyno- | | Nu- | atggaaggcatctccatctacacctccgacaactacaccgaggaaatgggctccggcgactacgact |
| | molgus | | cleic | ccatcaaagagccttgcttccgggaagagaacgcccacttcaaccggatctttctgcccacaatcta |
| | CXCR4 | | acid | ctctatcatcttcctgaccggcatcgtcggcaacggcctggtcattctggtcatgggctaccagaaa |
| | | | se- | aagctgcggtccatgaccgacaagtaccggctgcatctgtctgtggccgacctgctgttcgtgatca |
| | | | quence | ccctgcctttttgggccgtcgatgccgtggccaactggtacttcggcaacttcctgtgcaaggccgt |
| | | | of | gcacgtgatctacaccgtgaacctgtactcctccgtgctgatcctggccttcatctccctggacaga |
| | | | cyno- | tacctggccatcgtgcacgccaccaactctcagaagcccagaaagctgctggccgagaaggtggtgt |
| | | | molgus | atgtcggcgtgtggattcccgctctgctgctgacaatccccgacttcatcttcgcctccgtgtccga |
| | | | CXCR4 | ggccgacgaccggtacatctgcgacagattctaccccaacgacctgtgggtcgtcgtgttccagttc |
| | | | | cagcacatcatggtcggactgatcctgcctggcatcgtgatcctgtcctgctactgcatcatcatct |
| | | | | ccaagctgtcccactccaagggccaccagaagcggaaggctctgaaaaccacagtgatcctgattct |
| | | | | ggctttcttcgcctgctggctgccctactacatcggcatcagcatcgacagcttcatcctgctggaa |
| | | | | atcatcaagcagggctgcgagttcgagaacaccgtgcacaagtggatctccattaccgaggctctgg |
| | | | | ccttcttccactgttgtctgaaccctatcctgtacgccttcctgggcgccaagttcaagacctctgc |
| | | | | tcagcacgctctgacctctgtgtccagaggctccagcctgaagatcctgagcaagggcaagagaggc |
| | | | | ggccactcttctgtgtctaccgagtccgagtcctccagcttccactcctct |
| 10 | CL- | CDRH1 | Amino | GFTFYSYG |
| | 82574 | (IMGT) | acid | |
| | | | se- | |
| | | | quence | |
| | | | of | |
| | | | CDRH1 | |
| | | | of CL- | |
| | | | 82574 | |
| | | | using | |
| | | | IMGT | |
| 11 | CL- | CDRH2 | Amino | IWYGGTNK |
| | 82574 | (IMGT) | acid | |
| | | | se- | |
| | | | quence | |
| | | | of | |
| | | | CDRH2 | |
| | | | of CL- | |
| | | | 82574 | |
| | | | using | |
| | | | IMGT | |
| 12 | CL- | CDRH3 | Amino | ARGQLERRRGGLNI |
| | 82574 | (IMGT) | acid | |
| | | | se- | |
| | | | quence | |
| | | | of | |
| | | | CDRH3 | |
| | | | of CL- | |
| | | | 82574 | |
| | | | using | |
| | | | IMGT | |
| 13 | CL- | CDRH1 | Amino | SYGMH |
| | 82574 | (KABAT) | acid | |
| | | | se- | |
| | | | quence | |
| | | | of | |
| | | | CDRH1 | |
| | | | of CL- | |
| | | | 82574 | |
| | | | using | |
| | | | Kabat | |
| 14 | CL- | CDRH2 | Amino | VIWYGGTNKYYADSVKG |
| | 82574 | (KABAT) | acid | |
| | | | se- | |
| | | | quence | |
| | | | of | |
| | | | CDRH2 | |
| | | | of CL- | |
| | | | 82574 | |
| | | | using | |
| | | | Kabat | |
| 15 | CL- | CDRH3 | Amino | GQLERRRGGLNI |
| | 82574 | (KABAT) | acid | |
| | | | se- | |
| | | | quence | |
| | | | of | |
| | | | CDRH3 | |
| | | | of CL- | |
| | | | 82574 | |
| | | | using | |
| | | | Kabat | |
| 16 | CL- | Heavy | Amino | QVQLVESGGGVVQPGRSLRLSCAASGFTFYSYGMHWVRQAPGKGLEWVAVIWYGGTNKYYADS |
| | 82574 | chain | acid | VKGRFTISRDNSKNTLYLQMNSLTAEDTAVYYCARGQLERRRGGLNIWGQGTVVTVSS |
| | | vari- | se- | |
| | | able | quence | |
| | | region | of V_{H} | |
| | | | of | |
| | | | CL- | |
| | | | 82574 | |
| 17 | CL- | Heavy | Nu- | caggtgcagctggtggagtctgggggaggcgtggtccagcctgggaggtccctgagactctcctgtg |
| | 82574 | chain | cleic | cagcgtctggattcaccttctatagctatggcatgcactgggtccgccaggctccaggcaagggact |
| | | vari- | acid | ggagtgggtggcagttatatggtatggtggaactaataaatactatgcagactccgtgaagggccga |
| | | able | se- | ttcaccatctccagagacaattccaagaacacgctgtatctgcaaatgaacagcctgacagccgaag |
| | | region | quence | acacggctgtgtattactgtgcgagagggcagctggaacgccgacggggcggtttgaatatctgggg |
| | | | of V_{H} | ccaagggacagtggtcaccgtctcttcag |
| | | | of | |
| | | | CL- | |
| | | | 82574 | |
| 18 | CL- | Full | Amino | QVQLVESGGGVVQPGRSLRLSCAASGFTFYSYGMHWVRQAPGKGLEWVAVIWYGGTNKYYADS |
| | 82574 | heavy | acid | VKGRFTISRDNSKNTLYLQMNSLTAEDTAVYYCARGQLERRRGGLNIWGQGTVVTVSSASTKGP |
| | | chain | se- | SVFPLAPCSRSTSESTAALGCLVKDYFPEPVTVSWNSGALTSGVHTFPAVLQSSGLYSLSSVVTVP |
| | | se- | quence | SSSLGTKTYTCNVDHKPSNTKVDKRVESKYGPPCPPCPAPEFEGGPSVFLFPPKPKDTLMISRTPE |
| | | quence | of CL- | VTCVVVDVSQEDPEVQFNWYVDGVEVHNAKTKPREEQFNSTYRVVSVLTVLHQDWLNGKEYKC |
| | | | 82574 | KVSNKGLPSSIEKTISKAKGQPREPQVYTLPPSQEEMTKNQVSLTCLVKGFYPSDIAVEWESNGQP |
| | | | heavy | ENNYKTTPPVLDSDGSFFLYSRLTVDKSRWQEGNVFSCSVMHEALHNHYTQKSLSLSLGK |
| | | | chain | |
| 19 | CL- | Full | Nu- | caggtgcagctggtggagtctgggggaggcgtggtccagcctgggaggtccctgagactctcctgtg |
| | 82574 | heavy | cleic | cagcgtctggattcaccttctatagctatggcatgcactgggtccgccaggctccaggcaagggact |
| | | chain | acid | ggagtgggtggcagttatatggtatggtggaactaataaatactatgcagactccgtgaagggccga |
| | | se- | se- | ttcaccatctccagagacaattccaagaacacgctgtatctgcaaatgaacagcctgacagccgaag |
| | | quence | quence | acacggctgtgtattactgtgcgagagggcagctggaacgccgacggggcggtttgaatatctgggg |
| | | | of CL- | ccaagggacagtggtcaccgtctcttcagccagcaccaagggcccttccgtgttccccctggcccct |
| | | | 82574 | tgcagcaggagcacctccgaatccacagctgccctgggctgtctggtgaaggactactttcccgagc |
| | | | heavy | ccgtgaccgtgagctggaacagcggcgctctgacatccggcgtccacacctttcctgccgtcctgca |
| | | | chain | gtcctccggcctctactccctgtcctccgtggtgaccgtgcctagctcctccctcggcaccaagacc |
| | | | | tacacctgtaacgtggaccacaaaccctccaacaccaaggtggacaaacgggtcgagagcaagtacg |
| | | | | gccctccctgccctccttgtcctgcccccgagttcgaaggcggacccagcgtgttcctgttccctcc |
| | | | | taagcccaaggacaccctcatgatcagccggacacccgaggtgacctgcgtggtggtggatgtgagc |
| | | | | caggaggaccctgaggtccagttcaactggtatgtggatggcgtggaggtgcacaacgccaagacaa |
| | | | | agccccgggaagagcagttcaactccacctacagggtggtcagcgtgctgaccgtgctgcatcagga |
| | | | | ctggctgaacggcaaggagtacaagtgcaaggtcagcaataagggactgcccagcagcatcgagaag |
| | | | | accatctccaaggctaaaggccagccccgggaacctcaggtgtacaccctgcctcccagccaggagg |
| | | | | agatgaccaagaaccaggtgagcctgacctgcctggtgaagggattctacccttccgacatcgccgt |
| | | | | ggagtgggagtccaacggccagcccgagaacaattataagaccacccctcccgtcctcgacagcgac |
| | | | | ggatccttctttctgtactccaggctgaccgtggataagtccaggtggcaggaaggcaacgtgttca |
| | | | | gctgctccgtgatgcacgaggccctgcacaatcactacacccagaagtccctgagcctgtccctggg |
| | | | | aaag |
| 20 | CL- | CDRL1 | Amino | QGVRDD |
| | 82574 | (IMGT) | acid | |
| | | | se- | |
| | | | quence | |
| | | | of | |
| | | | CDRL1 | |
| | | | of CL- | |
| | | | 82574 | |
| | | | using | |
| | | | IMGT | |
| 21 | CL- | CDRL2 | Amino | AAS |
| | 82574 | (IMGT) | acid | |
| | | | se- | |
| | | | quence | |
| | | | of | |
| | | | CDRL2 | |
| | | | of CL- | |
| | | | 82574 | |
| | | | using | |
| | | | IMGT | |
| 22 | CL- | CDRL3 | Amino | LQHNSYPFT |
| | 82574 | (IMGT) | acid | |
| | | | se- | |
| | | | quence | |
| | | | of | |
| | | | CDRL3 | |
| | | | of CL- | |
| | | | 82574 | |
| | | | using | |
| | | | IMGT | |
| 23 | CL- | CDRL1 | Amino | RASQGVRDDLG |
| | 82574 | (KABAT) | acid | |
| | | | se- | |
| | | | quence | |
| | | | of | |
| | | | CDRL1 | |
| | | | of CL- | |
| | | | 82574 | |
| | | | using | |
| | | | KABAT | |
| 24 | CL- | CDRL2 | Amino | AASSLQS |
| | 82574 | (KABAT) | acid | |
| | | | se- | |
| | | | quence | |
| | | | of | |
| | | | CDRL2 | |
| | | | of CL- | |
| | | | 82574 | |
| | | | using | |
| | | | KABAT | |
| 25 | CL- | CDRL3 | Amino | LQHNSYPFT |
| | 82574 | (KABAT) | acid | |
| | | | se- | |
| | | | quence | |
| | | | of | |
| | | | CDRL3 | |
| | | | of CL- | |
| | | | 82574 | |
| | | | using | |
| | | | KABAT | |
| 26 | CL- | Light | Amino | DIQMTQSPSSLSASVGDRVTITCRASQGVRDDLGWYQQKPGKAPKRLIYAASSLQSGVPSRFSGS |
| | 82574 | chain | acid | GSGTEFTLTISSLQPEDFATYYCLQHNSYPFTFGPGTKVDIK |
| | | vari- | se- | |
| | | able | quence | |
| | | region | of V_{L} | |
| | | | of | |
| | | | CL- | |
| | | | 82574 | |
| 27 | CL- | Light | Nu- | gacatccagatgacccagtctccatcctccctgtctgcatctgtaggagacagagtcaccatcactt |
| | 82574 | chain | cleic | gccgggcaagtcagggcgttagagatgatttaggttggtatcagcagaaaccagggaaagcccctaa |
| | | vari- | acid | gcgcctgatctatgctgcatccagtttgcaaagtggggtcccatcaaggttcagcggcagtggatct |
| | | able | se- | gggacagaattcactctcacaatcagcagcctgcagcctgaagattttgcaacttattactgtctac |
| | | region | quence | agcataatagttatccattcactttcggccctgggaccaaagtggatatcaaac |
| | | | of V_{L} | |
| | | | of | |
| | | | CL- | |
| | | | 82574 | |
| 28 | CL- | Full | Amino | DIQMTQSPSSLSASVGDRVTITCRASQGVRDDLGWYQQKPGKAPKRLIYAASSLQSGVPSRFSGS |
| | 82574 | light | acid | GSGTEFTLTISSLQPEDFATYYCLQHNSYPFTFGPGTKVDIKRTVAAPSVFIFPPSDEQLKSGTASV |
| | | chain | se- | VCLLNNFYPREAKVQWKVDNALQSGNSQESVTEQDSKDSTYSLSSTLTLSKADYEKHKVYACEVT |
| | | se- | quence | HQGLSSPVTKSFNRGEC |
| | | quence | of CL- | |
| | | | 82574 | |
| | | | light | |
| | | | chain | |
| 29 | CL- | Full | Nu- | gacatccagatgacccagtctccatcctccctgtctgcatctgtaggagacagagtcaccatcactt |
| | 82574 | light | cleic | gccgggcaagtcagggcgttagagatgatttaggttggtatcagcagaaaccagggaaagcccctaa |
| | | chain | acid | gcgcctgatctatgctgcatccagtttgcaaagtggggtcccatcaaggttcagcggcagtggatct |
| | | se- | se- | gggacagaattcactctcacaatcagcagcctgcagcctgaagattttgcaacttattactgtctac |
| | | quence | quence | agcataatagttatccattcactttcggccctgggaccaaagtggatatcaaacgtacggtggccgc |
| | | | of CL- | tccctccgtgttcatcttcccaccttccgacgagcagctgaagtccggcaccgcttctgtcgtgtgc |
| | | | 82574 | ctgctgaacaacttctacccccgcgaggccaaggtgcagtggaaggtggacaacgccctgcagtccg |
| | | | light | gcaactcccaggaatccgtgaccgagcaggactccaaggacagcacctactccctgtcctccaccct |
| | | | chain | gaccctgtccaaggccgactacgagaagcacaaggtgtacgcctgcgaagtgacccaccagggcctg |
| | | | | tctagccccgtgaccaagtctttcaaccggggcgagtgt |
| 30 | CL- | CDRH1 | Amino | GFTFYSYA |
| | 82458 | (IMGT) | acid | |
| | | | se- | |
| | | | quence | |
| | | | of | |
| | | | CDRH1 | |
| | | | of CL- | |
| | | | 82458 | |
| | | | using | |
| | | | IMGT | |
| 31 | CL- | CDRH2 | Amino | ISGSGDIT |
| | 82458 | (IMGT) | acid | |
| | | | se- | |
| | | | quence | |
| | | | of | |
| | | | CDRH2 | |
| | | | of CL- | |
| | | | 82458 | |
| | | | using | |
| | | | IMGT | |
| 32 | CL- | CDRH3 | Amino | AKGQLERRYYKYYGMDV |
| | 82458 | (IMGT) | acid | |
| | | | se- | |
| | | | quence | |
| | | | of | |
| | | | CDRH3 | |
| | | | of CL- | |
| | | | 82458 | |
| | | | using | |
| | | | IMGT | |
| 33 | CL- | CDRH1 | Amino | SYAMN |
| | 82458 | (KABAT) | acid | |
| | | | se- | |
| | | | quence | |
| | | | of | |
| | | | CDRH1 | |
| | | | of CL- | |
| | | | 82458 | |
| | | | using | |
| | | | Kabat | |
| 34 | CL- | CDRH2 | Amino | GISGSGDITYYADSVKG |
| | 82458 | (KABAT) | acid | |
| | | | se- | |
| | | | quence | |
| | | | of | |
| | | | CDRH2 | |
| | | | of CL- | |
| | | | 82458 | |
| | | | using | |
| | | | Kabat | |
| 35 | CL- | CDRH3 | Amino | GQLERRYYKYYGMDV |
| | 82458 | (KABAT) | acid | |
| | | | se- | |
| | | | quence | |
| | | | of | |
| | | | CDRH3 | |
| | | | of CL- | |
| | | | 82458 | |
| | | | using | |
| | | | Kabat | |
| 36 | CL- | Heavy | Amino | EVQLVESGGDLVQPGGSLRLSCAASGFTFYSYAMNWVRQAPGKGLKWVSGISGSGDITYYADSV |
| | 82458 | chain | acid | KGRFTISRDNSKNTLYLQMHSLRPEDTAIYYCAKGQLERRYYKYYGMDVWGQGTTVTVSS |
| | | vari- | se- | |
| | | able | quence | |
| | | region | of V_{H} | |
| | | | of | |
| | | | CL- | |
| | | | 82458 | |
| 37 | CL- | Heavy | Nu- | gaggtgcagctggtagagtctgggggagacttagtacagcctggggggtccctgagactctcttgtg |
| | 82458 | chain | cleic | cagcctctggattcaccttttacagctatgccatgaattgggtccgccaggctccagggaaggggct |
| | | vari- | acid | aaaatgggtctcaggtattagtggtagtggtgatatcacatactacgcagactccgtgaagggccgg |
| | | able | se- | ttcaccatctccagagacaattccaagaacacgctgtatctgcaaatgcacagcctgagacccgaag |
| | | region | quence | acacggccatatattactgtgcgaaaggccaactggaacgacggtactacaagtactacggtatgga |
| | | | of V_{H} | cgtctggggccaagggaccacggtcaccgtctcctcag |
| | | | of | |
| | | | CL- | |
| | | | 82458 | |
| 38 | CL- | Full | Amino | EVQLVESGGDLVQPGGSLRLSCAASGFTFYSYAMNWVRQAPGKGLKWVSGISGSGDITYYADSV |
| | 82458 | heavy | acid | KGRFTISRDNSKNTLYLQMHSLRPEDTAIYYCAKGQLERRYYKYYGMDVWGQGTTVTVSSASTK |
| | | chain | se- | GPSVFPLAPCSRSTSESTAALGCLVKDYFPEPVTVSWNSGALTSGVHTFPAVLQSSGLYSLSSVVT |
| | | se- | quence | VPSSSLGTKTYTCNVDHKPSNTKVDKRVESKYGPPCPPCPAPEFEGGPSVFLFPPKPKDTLMISRT |
| | | quence | of CL- | PEVTCVVVDVSQEDPEVQFNWYVDGVEVHNAKTKPREEQFNSTYRVVSVLTVLHQDWLNGKEY |
| | | | 82458 | KCKVSNKGLPSSIEKTISKAKGQPREPQVYTLPPSQEEMTKNQVSLTCLVKGFYPSDIAVEWESNG |
| | | | heavy | QPENNYKTTPPVLDSDGSFFLYSRLTVDKSRWQEGNVFSCSVMHEALHNHYTQKSLSLSLGK |
| | | | chain | |
| 39 | CL- | Full | Nu- | gaggtgcagctggtagagtctgggggagacttagtacagcctggggggtccctgagactctcttgtg |
| | 82458 | heavy | cleic | cagcctctggattcaccttttacagctatgccatgaattgggtccgccaggctccagggaaggggct |
| | | chain | acid | aaaatgggtctcaggtattagtggtagtggtgatatcacatactacgcagactccgtgaagggccgg |
| | | se- | se- | ttcaccatctccagagacaattccaagaacacgctgtatctgcaaatgcacagcctgagacccgaag |
| | | quence | quence | acacggccatatattactgtgcgaaaggccaactggaacgacggtactacaagtactacggtatgga |
| | | | of CL- | cgtctggggccaagggaccacggtcaccgtctcctcagccagcaccaagggcccttccgtgttcccc |
| | | | 82458 | ctggccccttgcagcaggagcacctccgaatccacagctgccctgggctgtctggtgaaggactact |
| | | | heavy | ttcccgagcccgtgaccgtgagctggaacagcggcgctctgacatccggcgtccacacctttcctgc |
| | | | chain | cgtcctgcagtcctccggcctctactccctgtcctccgtggtgaccgtgcctagctcctccctcggc |
| | | | | accaagacctacacctgtaacgtggaccacaaaccctccaacaccaaggtggacaaacgggtcgaga |
| | | | | gcaagtacggccctccctgccctccttgtcctgcccccgagttcgaaggcggacccagcgtgttcct |
| | | | | gttccctcctaagcccaaggacaccctcatgatcagccggacacccgaggtgacctgcgtggtggtg |
| | | | | gatgtgagccaggaggaccctgaggtccagttcaactggtatgtggatggcgtggaggtgcacaacg |
| | | | | ccaagacaaagccccgggaagagcagttcaactccacctacagggtggtcagcgtgctgaccgtgct |
| | | | | gcatcaggactggctgaacggcaaggagtacaagtgcaaggtcagcaataagggactgcccagcagc |
| | | | | atcgagaagaccatctccaaggctaaaggccagccccgggaacctcaggtgtacaccctgcctccca |
| | | | | gccaggaggagatgaccaagaaccaggtgagcctgacctgcctggtgaagggattctacccttccga |
| | | | | catcgccgtggagtgggagtccaacggccagcccgagaacaattataagaccacccctcccgtcctc |
| | | | | gacagcgacggatccttctttctgtactccaggctgaccgtggataagtccaggtggcaggaaggca |
| | | | | acgtgttcagctgctccgtgatgcacgaggccctgcacaatcactacacccagaagtccctgagcct |
| | | | | gtccctgggaaag |
| 40 | CL- | CDRL1 | Amino | QGIRND |
| | 82458 | (IMGT) | acid | |
| | | | se- | |
| | | | quence | |
| | | | of | |
| | | | CDRL1 | |
| | | | of CL- | |
| | | | 82458 | |
| | | | using | |
| | | | IMGT | |
| 41 | CL- | CDRL2 | Amino | GAS |
| | 82458 | (IMGT) | acid | |
| | | | se- | |
| | | | quence | |
| | | | of | |
| | | | CDRL2 | |
| | | | of CL- | |
| | | | 82458 | |
| | | | using | |
| | | | IMGT | |
| 42 | CL- | CDRL3 | Amino | LQQNSYPLT |
| | 82458 | (IMGT) | acid | |
| | | | se- | |
| | | | quence | |
| | | | of | |
| | | | CDRL3 | |
| | | | of A | |
| | | | CL- | |
| | | | 82458 | |
| | | | using | |
| | | | IMGT | |
| 43 | CL- | CDRL1 | Amino | RANQGIRNDLG |
| | 82458 | (KABAT) | acid | |
| | | | se- | |
| | | | quence | |
| | | | of | |
| | | | CDRL1 | |
| | | | of CL- | |
| | | | 82458 | |
| | | | using | |
| | | | KABAT | |
| 44 | CL- | CDRL2 | Amino | GASSLKS |
| | 82458 | (KABAT) | acid | |
| | | | se- | |
| | | | quence | |
| | | | of | |
| | | | CDRL2 | |
| | | | of CL- | |
| | | | 82458 | |
| | | | using | |
| | | | KABAT | |
| 45 | CL- | CDRL3 | Amino | LQQNSYPLT |
| | 82458 | (KABAT) | acid | |
| | | | se- | |
| | | | quence | |
| | | | of | |
| | | | CDRL3 | |
| | | | of CL- | |
| | | | 82458 | |
| | | | using | |
| | | | KABAT | |
| 46 | CL- | Light | Amino | DIQMTQSPSSLSASVGDRVTITCRANQGIRNDLGWYQQKPGKAPKRLIYGASSLKSGVPSRFSGS |
| | 82458 | chain | acid | GSGTEFTLTISSLQPEDFATYYCLQQNSYPLTFGGGTKVEIK |
| | | vari- | se- | |
| | | able | quence | |
| | | region | of V_{L} | |
| | | | of | |
| | | | CL- | |
| | | | 82458 | |
| 47 | CL- | Light | Nu- | gacatccagatgacccagtctccatcctccctgtctgcatctgtaggagacagagtcaccatcactt |
| | 82458 | chain | cleic | gccgggcaaatcagggcattagaaatgatttaggctggtatcagcagaaaccagggaaagcccctaa |
| | | vari- | acid | gcgcctgatctatggtgcatccagtttgaaaagtggggtcccatcaaggttcagcggcagtggatct |
| | | able | se- | gggacagaattcactctcacaatcagcagcctgcagcctgaagattttgcaacctactattgtctac |
| | | region | quence | agcagaatagttacccgctcactttcggcggagggaccaaagtggagatcaaac |
| | | | of V_{L} | |
| | | | of | |
| | | | CL- | |
| | | | 82458 | |
| 48 | CL- | Full | Amino | DIQMTQSPSSLSASVGDRVTITCRANQGIRNDLGWYQQKPGKAPKRLIYGASSLKSGVPSRFSGS |
| | 82458 | light | acid | GSGTEFTLTISSLQPEDFATYYCLQQNSYPLTFGGGTKVEIKRTVAAPSVFIFPPSDEQLKSGTASV |
| | | chain | se- | VCLLNNFYPREAKVQWKVDNALQSGNSQESVTEQDSKDSTYSLSSTLTLSKADYEKHKVYACEVT |
| | | se- | quence | HQGLSSPVTKSFNRGEC |
| | | quence | of CL- | |
| | | | 82458 | |
| | | | light | |
| | | | chain | |
| 49 | CL- | Full | Nu- | gacatccagatgacccagtctccatcctccctgtctgcatctgtaggagacagagtcaccatcactt |
| | 82458 | light | cleic | gccgggcaaatcagggcattagaaatgatttaggctggtatcagcagaaaccagggaaagcccctaa |
| | | chain | acid | gcgcctgatctatggtgcatccagtttgaaaagtggggtcccatcaaggttcagcggcagtggatct |
| | | se- | se- | gggacagaattcactctcacaatcagcagcctgcagcctgaagattttgcaacctactattgtctac |
| | | quence | quence | agcagaatagttacccgctcactttcggcggagggaccaaagtggagatcaaacgtacggtggccgc |
| | | | of CL- | tccctccgtgttcatcttcccaccttccgacgagcagctgaagtccggcaccgcttctgtcgtgtgc |
| | | | 82458 | ctgctgaacaacttctacccccgcgaggccaaggtgcagtggaaggtggacaacgccctgcagtccg |
| | | | light | gcaactcccaggaatccgtgaccgagcaggactccaaggacagcacctactccctgtcctccaccct |
| | | | chain | gaccctgtccaaggccgactacgagaagcacaaggtgtacgcctgcgaagtgacccaccagggcctg |
| | | | | tctagccccgtgaccaagtctttcaaccggggcgagtgt |
| 50 | CL- | CDRH1 | Amino | GGSISSGGYY |
| | 82558 | (IMGT) | acid | |
| | | | se- | |
| | | | quence | |
| | | | of | |
| | | | CDRH1 | |
| | | | of CL- | |
| | | | 82558 | |
| | | | using | |
| | | | IMGT | |
| 51 | CL- | CDRH2 | Amino | ISYSGNT |
| | 82558 | (IMGT) | acid | |
| | | | se- | |
| | | | quence | |
| | | | of | |
| | | | CDRH2 | |
| | | | of CL- | |
| | | | 82558 | |
| | | | using | |
| | | | IMGT | |
| 52 | CL- | CDRH3 | Amino | ARDRRIPMLRGVKGYYYGLDV |
| | 82558 | (IMGT) | acid | |
| | | | se- | |
| | | | quence | |
| | | | of | |
| | | | CDRH3 | |
| | | | of CL- | |
| | | | 82558 | |
| | | | using | |
| | | | IMGT | |
| 53 | CL- | CDRH1 | Amino | SGGYYWS |
| | 82558 | (KABAT) | acid | |
| | | | se- | |
| | | | quence | |
| | | | of | |
| | | | CDRH1 | |
| | | | of CL- | |
| | | | 82558 | |
| | | | using | |
| | | | Kabat | |
| 54 | CL- | CDRH2 | Amino | YISYSGNTFYSPSLKS |
| | 82558 | (KABAT) | acid | |
| | | | se- | |
| | | | quence | |
| | | | of | |
| | | | CDRH2 | |
| | | | of CL- | |
| | | | 82558 | |
| | | | using | |
| | | | Kabat | |
| 55 | CL- | CDRH3 | Amino | DRRIPMLRGVKGYYYGLDV |
| | 82558 | (KABAT) | acid | |
| | | | se- | |
| | | | quence | |
| | | | of | |
| | | | CDRH3 | |
| | | | of CL- | |
| | | | 82558 | |
| | | | using | |
| | | | Kabat | |
| 56 | CL- | Heavy | Amino | QVQLQESGPGLVKPSQTLSLTCTVSGGSISSGGYYWSWIRQVPGKGLEWIGYISYSGNTFYSPSL |
| | 82558 | chain | acid | KSRVTISVDTSENKFSLRLTSVTAADTAVYFCARDRRIPMLRGVKGYYYGLDVWGQGTTVTVSS |
| | | vari- | se- | |
| | | able | quence | |
| | | region | of V_{H} | |
| | | | of | |
| | | | CL- | |
| | | | 82558 | |
| 57 | CL- | Heavy | Nu- | caggtgcagctgcaggagtcgggcccaggactggtgaagccttcacagaccctgtccctcacctgca |
| | 82558 | chain | cleic | ctgtctctggtggctccatcagcagtggtgggtactactggagctggatccgccaggtcccagggaa |
| | | vari- | acid | gggcctggagtggattggatacatctcatacagtgggaacaccttctacagcccgtccctcaagagt |
| | | able | se- | cgagttaccatatcagtagacacgtctgagaacaagttctccctgaggctgacctctgtgactgccg |
| | | region | quence | cggacacggccgtgtatttctgtgcgagagatcgacgtattcctatgcttcggggagttaaaggcta |
| | | | of V_{H} | ctactacggtttggacgtctggggccaagggaccacggtcaccgtctcctcag |
| | | | of | |
| | | | CL- | |
| | | | 82558 | |
| 58 | CL- | Full | Amino | QVQLQESGPGLVKPSQTLSLTCTVSGGSISSGGYYWSWIRQVPGKGLEWIGYISYSGNTFYSPSL |
| | 82558 | heavy | acid | KSRVTISVDTSENKFSLRLTSVTAADTAVYFCARDRRIPMLRGVKGYYYGLDVWGQGTTVTVSSA |
| | | chain | se- | STKGPSVFPLAPCSRSTSESTAALGCLVKDYFPEPVTVSWNSGALTSGVHTFPAVLQSSGLYSLSS |
| | | se- | quence | VVTVPSSSLGTKTYTCNVDHKPSNTKVDKRVESKYGPPCPPCPAPEFEGGPSVFLFPPKPKDTLMI |
| | | quence | of CL- | SRTPEVTCVVVDVSQEDPEVQFNWYVDGVEVHNAKTKPREEQFNSTYRVVSVLTVLHQDWLNG |
| | | | 82558 | KEYKCKVSNKGLPSSIEKTISKAKGQPREPQVYTLPPSQEEMTKNQVSLTCLVKGFYPSDIAVEWE |
| | | | heavy | SNGQPENNYKTTPPVLDSDGSFFLYSRLTVDKSRWQEGNVFSCSVMHEALHNHYTQKSLSLSLG |
| | | | chain | K |
| 59 | CL- | Full | Nu- | caggtgcagctgcaggagtcgggcccaggactggtgaagccttcacagaccctgtccctcacctgca |
| | 82558 | heavy | cleic | ctgtctctggtggctccatcagcagtggtgggtactactggagctggatccgccaggtcccagggaa |
| | | chain | acid | gggcctggagtggattggatacatctcatacagtgggaacaccttctacagcccgtccctcaagagt |
| | | se- | se- | cgagttaccatatcagtagacacgtctgagaacaagttctccctgaggctgacctctgtgactgccg |
| | | quence | quence | cggacacggccgtgtatttctgtgcgagagatcgacgtattcctatgcttcggggagttaaaggcta |
| | | | of CL- | ctactacggtttggacgtctggggccaagggaccacggtcaccgtctcctcagccagcaccaagggc |
| | | | 82558 | ccttccgtgttccccctggccccttgcagcaggagcacctccgaatccacagctgccctgggctgtc |
| | | | heavy | tggtgaaggactactttcccgagcccgtgaccgtgagctggaacagcggcgctctgacatccggcgt |
| | | | chain | ccacacctttcctgccgtcctgcagtcctccggcctctactccctgtcctccgtggtgaccgtgcct |
| | | | | agctcctccctcggcaccaagacctacacctgtaacgtggaccacaaaccctccaacaccaaggtgg |
| | | | | acaaacgggtcgagagcaagtacggccctccctgccctccttgtcctgcccccgagttcgaaggcgg |
| | | | | acccagcgtgttcctgttccctcctaagcccaaggacaccctcatgatcagccggacacccgaggtg |
| | | | | acctgcgtggtggtggatgtgagccaggaggaccctgaggtccagttcaactggtatgtggatggcg |
| | | | | tggaggtgcacaacgccaagacaaagccccgggaagagcagttcaactccacctacagggtggtcag |
| | | | | cgtgctgaccgtgctgcatcaggactggctgaacggcaaggagtacaagtgcaaggtcagcaataag |
| | | | | ggactgcccagcagcatcgagaagaccatctccaaggctaaaggccagccccgggaacctcaggtgt |
| | | | | acaccctgcctcccagccaggaggagatgaccaagaaccaggtgagcctgacctgcctggtgaaggg |
| | | | | attctacccttccgacatcgccgtggagtgggagtccaacggccagcccgagaacaattataagacc |
| | | | | acccctcccgtcctcgacagcgacggatccttctttctgtactccaggctgaccgtggataagtcca |
| | | | | ggtggcaggaaggcaacgtgttcagctgctccgtgatgcacgaggccctgcacaatcactacaccca |
| | | | | gaagtccctgagcctgtccctgggaaag |
| 60 | CL- | CDRL1 | Amino | QSLLHRNGYKY |
| | 82558 | (IMGT) | acid | |
| | | | se- | |
| | | | quence | |
| | | | of | |
| | | | CDRL1 | |
| | | | of CL- | |
| | | | 82558 | |
| | | | using | |
| | | | IMGT | |
| 61 | CL- | CDRL2 | Amino | LGS |
| | 82558 | (IMGT) | acid | |
| | | | se- | |
| | | | quence | |
| | | | of | |
| | | | CDRL2 | |
| | | | of CL- | |
| | | | 82558 | |
| | | | using | |
| | | | IMGT | |
| 62 | CL- | CDRL3 | Amino | MQALQTPWT |
| | 82558 | (IMGT) | acid | |
| | | | se- | |
| | | | quence | |
| | | | of | |
| | | | CDRL3 | |
| | | | of CL- | |
| | | | 82558 | |
| | | | using | |
| | | | IMGT | |
| 63 | CL- | CDRL1 | Amino | RSSQSLLHRNGYKYLD |
| | 82558 | (KABAT) | acid | |
| | | | se- | |
| | | | quence | |
| | | | of | |
| | | | CDRL1 | |
| | | | of CL- | |
| | | | 82558 | |
| | | | using | |
| | | | KABAT | |
| 64 | CL- | CDRL2 | Amino | LGSNRAS |
| | 82558 | (KABAT) | acid | |
| | | | se- | |
| | | | quence | |
| | | | of | |
| | | | CDRL2 | |
| | | | of CL- | |
| | | | 82558 | |
| | | | using | |
| | | | KABAT | |
| 65 | CL- | CDRL3 | Amino | MQALQTPWT |
| | 82558 | (KABAT) | acid | |
| | | | se- | |
| | | | quence | |
| | | | of | |
| | | | CDRL3 | |
| | | | of CL- | |
| | | | 82558 | |
| | | | using | |
| | | | KABAT | |
| 66 | CL- | Light | Amino | DIVMTQSPLSLPVIPGEPASISCRSSQSLLHRNGYKYLDWYLQKPGQSPQLLIYLGSNRASGVPAR |
| | 82558 | chain | acid | FSGSGSGTDFTLKISRVEAEDVGVYYCMQALQTPWTFGQGTKVEIK |
| | | vari- | se- | |
| | | able | quence | |
| | | region | of V_{L} | |
| | | | of | |
| | | | CL- | |
| | | | 82558 | |
| 67 | CL- | Light | Nu- | gatattgtgatgactcagtctccactctccctgcccgtcatccctggagagccggcctccatctcct |
| | 82558 | chain | cleic | gcaggtctagtcagagcctcctgcatagaaatggatacaagtatttggattggtacctgcagaagcc |
| | | vari- | acid | ggggcagtctccacagctcctgatctatttgggttctaatcgggcctccggggtccctgccaggttc |
| | | able | se- | agtggcagtggatcaggcacagattttacactgaaaatcagcagagtggaggctgaggatgttgggg |
| | | region | quence | tttattactgtatgcaagctctacaaactccgtggacgttcggccaagggaccaaggtggaaatcaa |
| | | | of V_{L} | ac |
| | | | of | |
| | | | CL- | |
| | | | 82558 | |
| 68 | CL- | Full | Amino | DIVMTQSPLSLPVIPGEPASISCRSSQSLLHRNGYKYLDWYLQKPGQSPQLLIYLGSNRASGVPAR |
| | 82558 | light | acid | FSGSGSGTDFTLKISRVEAEDVGVYYCMQALQTPWTFGQGTKVEIKRTVAAPSVFIFPPSDEQLK |
| | | chain | se- | SGTASVVCLLNNFYPREAKVQWKVDNALQSGNSQESVTEQDSKDSTYSLSSTLTLSKADYEKHK |
| | | se- | quence | VYACEVTHQGLSSPVTKSFNRGEC |
| | | quence | of CL- | |
| | | | 82558 | |
| | | | light | |
| | | | chain | |
| 69 | CL- | Full | Nu- | gatattgtgatgactcagtctccactctccctgcccgtcatccctggagagccggcctccatctcct |
| | 82558 | light | cleic | gcaggtctagtcagagcctcctgcatagaaatggatacaagtatttggattggtacctgcagaagcc |
| | | chain | acid | ggggcagtctccacagctcctgatctatttgggttctaatcgggcctccggggtccctgccaggttc |
| | | se- | se- | agtggcagtggatcaggcacagattttacactgaaaatcagcagagtggaggctgaggatgttgggg |
| | | quence | quence | tttattactgtatgcaagctctacaaactccgtggacgttcggccaagggaccaaggtggaaatcaa |
| | | | of CL- | acgtacggtggccgctccctccgtgttcatcttcccaccttccgacgagcagctgaagtccggcacc |
| | | | 82558 | gcttctgtcgtgtgcctgctgaacaacttctacccccgcgaggccaaggtgcagtggaaggtggaca |
| | | | light | acgccctgcagtccggcaactcccaggaatccgtgaccgagcaggactccaaggacagcacctactc |
| | | | chain | cctgtcctccaccctgaccctgtccaaggccgactacgagaagcacaaggtgtacgcctgcgaagtg |
| | | | | acccaccagggcctgtctagccccgtgaccaagtctttcaaccggggcgagtgt |
| 70 | CL- | CDRH1 | Amino | GFSFSSYG |
| | 82658 | (IMGT) | acid | |
| | | | se- | |
| | | | quence | |
| | | | of | |
| | | | CDRH1 | |
| | | | of CL- | |
| | | | 82658 | |
| | | | using | |
| | | | IMGT | |
| 71 | CL- | CDRH2 | Amino | ISYDGSNK |
| | 82658 | (IMGT) | acid | |
| | | | se- | |
| | | | quence | |
| | | | of | |
| | | | CDRH2 | |
| | | | of CL- | |
| | | | 82658 | |
| | | | using | |
| | | | IMGT | |
| 72 | CL- | CDRH3 | Amino | SKGSLTPRRGYYYSYGMDV |
| | 82658 | (IMGT) | acid | |
| | | | se- | |
| | | | quence | |
| | | | of | |
| | | | CDRH3 | |
| | | | of CL- | |
| | | | 82658 | |
| | | | using | |
| | | | IMGT | |
| 73 | CL- | CDRH1 | Amino | SYGMH |
| | 82658 | (KABAT) | acid | |
| | | | se- | |
| | | | quence | |
| | | | of | |
| | | | CDRH1 | |
| | | | of CL- | |
| | | | 82658 | |
| | | | using | |
| | | | Kabat | |
| 74 | CL- | CDRH2 | Amino | VISYDGSNKYYADSVKG |
| | 82658 | (KABAT) | acid | |
| | | | se- | |
| | | | quence | |
| | | | of | |
| | | | CDRH2 | |
| | | | of CL- | |
| | | | 82658 | |
| | | | using | |
| | | | Kabat | |
| 75 | CL- | CDRH3 | Amino | GSLTPRRGYYYSYGMDV |
| | 82658 | (KABAT) | acid | |
| | | | se- | |
| | | | quence | |
| | | | of | |
| | | | CDRH3 | |
| | | | of CL- | |
| | | | 82658 | |
| | | | using | |
| | | | Kabat | |
| 76 | CL- | Heavy | Amino | QVQLVESGGGVVQPGRSLRLSCAASGFSFSSYGMHWVRQAPGKGLEWVAVISYDGSNKYYADS |
| | 82658 | chain | acid | VKGRFTISRDNSKNTLYLQMNSLRGEDTAVYYCSKGSLTPRRGYYYSYGMDVWGQGTTVTVSS |
| | | vari- | se- | |
| | | able | quence | |
| | | region | of V_{H} | |
| | | | of | |
| | | | CL- | |
| | | | 82658 | |
| 77 | CL- | Heavy | Nu- | caggtgcagctggtggagtctgggggaggcgtggtccagcctgggaggtccctgagactctcctgtg |
| | 82658 | chain | cleic | cagcctctggattctccttcagtagctatggcatgcactgggtccgccaggctccaggcaaggggct |
| | | vari- | acid | ggagtgggtggcagttatatcatatgatggaagtaataaatactatgcagactccgtgaagggccga |
| | | able | se- | ttcaccatctccagagacaattccaagaacacgctgtatctgcaaatgaacagcctgagaggtgagg |
| | | region | quence | acacggctgtgtattactgttcgaaagggagtttaacacctcgccggggttactactactcctacgg |
| | | | of V_{H} | tatggacgtctggggccaagggaccacggtcaccgtctcctcag |
| | | | of | |
| | | | CL- | |
| | | | 82658 | |
| 78 | CL- | Full | Amino | QVQLVESGGGVVQPGRSLRLSCAASGFSFSSYGMHWVRQAPGKGLEWVAVISYDGSNKYYADS |
| | 82658 | heavy | acid | VKGRFTISRDNSKNTLYLQMNSLRGEDTAVYYCSKGSLTPRRGYYYSYGMDVWGQGTTVTVSSA |
| | | chain | se- | STKGPSVFPLAPCSRSTSESTAALGCLVKDYFPEPVTVSWNSGALTSGVHTFPAVLQSSGLYSLSS |
| | | se- | quence | VVTVPSSSLGTKTYTCNVDHKPSNTKVDKRVESKYGPPCPPCPAPEFEGGPSVFLFPPKPKDTLMI |
| | | quence | of CL- | SRTPEVTCVVVDVSQEDPEVQFNWYVDGVEVHNAKTKPREEQFNSTYRVVSVLTVLHQDWLNG |
| | | | 82658 | KEYKCKVSNKGLPSSIEKTISKAKGQPREPQVYTLPPSQEEMTKNQVSLTCLVKGFYPSDIAVEWE |
| | | | heavy | SNGQPENNYKTTPPVLDSDGSFFLYSRLTVDKSRWQEGNVFSCSVMHEALHNHYTQKSLSLSLG |
| | | | chain | K |
| 79 | CL- | Full | Nu- | caggtgcagctggtggagtctgggggaggcgtggtccagcctgggaggtccctgagactctcctgtg |
| | 82658 | heavy | cleic | cagcctctggattctccttcagtagctatggcatgcactgggtccgccaggctccaggcaaggggct |
| | | chain | acid | ggagtgggtggcagttatatcatatgatggaagtaataaatactatgcagactccgtgaagggccga |
| | | se- | se- | ttcaccatctccagagacaattccaagaacacgctgtatctgcaaatgaacagcctgagaggtgagg |
| | | quence | quence | acacggctgtgtattactgttcgaaagggagtttaacacctcgccggggttactactactcctacgg |
| | | | of CL- | tatggacgtctggggccaagggaccacggtcaccgtctcctcagccagcaccaagggcccttccgtg |
| | | | 82658 | ttccccctggccccttgcagcaggagcacctccgaatccacagctgccctgggctgtctggtgaagg |
| | | | heavy | actactttcccgagcccgtgaccgtgagctggaacagcggcgctctgacatccggcgtccacacctt |
| | | | chain | tcctgccgtcctgcagtcctccggcctctactccctgtcctccgtggtgaccgtgcctagctcctcc |
| | | | | ctcggcaccaagacctacacctgtaacgtggaccacaaaccctccaacaccaaggtggacaaacggg |
| | | | | tcgagagcaagtacggccctccctgccctccttgtcctgcccccgagttcgaaggcggacccagcgt |
| | | | | gttcctgttccctcctaagcccaaggacaccctcatgatcagccggacacccgaggtgacctgcgtg |
| | | | | gtggtggatgtgagccaggaggaccctgaggtccagttcaactggtatgtggatggcgtggaggtgc |
| | | | | acaacgccaagacaaagccccgggaagagcagttcaactccacctacagggtggtcagcgtgctgac |
| | | | | cgtgctgcatcaggactggctgaacggcaaggagtacaagtgcaaggtcagcaataagggactgccc |
| | | | | agcagcatcgagaagaccatctccaaggctaaaggccagccccgggaacctcaggtgtacaccctgc |
| | | | | ctcccagccaggaggagatgaccaagaaccaggtgagcctgacctgcctggtgaagggattctaccc |
| | | | | ttccgacatcgccgtggagtgggagtccaacggccagcccgagaacaattataagaccacccctccc |
| | | | | gtcctcgacagcgacggatccttctttctgtactccaggctgaccgtggataagtccaggtggcagg |
| | | | | aaggcaacgtgttcagctgctccgtgatgcacgaggccctgcacaatcactacacccagaagtccct |
| | | | | gagcctgtccctgggaaag |
| 80 | CL- | CDRL1 | Amino | QGIRND |
| | 82658 | (IMGT) | acid | |
| | | | se- | |
| | | | quence | |
| | | | of | |
| | | | CDRL1 | |
| | | | of CL- | |
| | | | 82658 | |
| | | | using | |
| | | | IMGT | |
| 81 | CL- | CDRL2 | Amino | AAS |
| | 82658 | (IMGT) | acid | |
| | | | se- | |
| | | | quence | |
| | | | of | |
| | | | CDRL2 | |
| | | | of CL- | |
| | | | 82658 | |
| | | | using | |
| | | | IMGT | |
| 82 | CL- | CDRL3 | Amino | LQHNGYPFT |
| | 82658 | (IMGT) | acid | |
| | | | se- | |
| | | | quence | |
| | | | of | |
| | | | CDRL3 | |
| | | | of CL- | |
| | | | 82658 | |
| | | | using | |
| | | | IMGT | |
| 83 | CL- | CDRL1 | Amino | RASQGIRNDLS |
| | 82658 | (KABAT) | acid | |
| | | | se- | |
| | | | quence | |
| | | | of | |
| | | | CDRL1 | |
| | | | of CL- | |
| | | | 82658 | |
| | | | using | |
| | | | KABAT | |
| 84 | CL- | CDRL2 | Amino | AASSLQS |
| | 82658 | (KABAT) | acid | |
| | | | se- | |
| | | | quence | |
| | | | of | |
| | | | CDRL2 | |
| | | | of CL- | |
| | | | 82658 | |
| | | | using | |
| | | | KABAT | |
| 85 | CL- | CDRL3 | Amino | LQHNGYPFT |
| | 82658 | (KABAT) | acid | |
| | | | se- | |
| | | | quence | |
| | | | of | |
| | | | CDRL3 | |
| | | | of CL- | |
| | | | 82658 | |
| | | | using | |
| | | | KABAT | |
| 86 | CL- | Light | Amino | DIQMTQSPSSLSASVGDRVTITCRASQGIRNDLSWYQQKPGKAPKRLIYAASSLQSGVPSRFSGS |
| | 82658 | chain | acid | GSGTEFTLTISSLQPEDFATYYCLQHNGYPFTFGPGTKVDIK |
| | | vari- | se- | |
| | | able | quence | |
| | | region | of V_{L} | |
| | | | of | |
| | | | CL- | |
| | | | 82658 | |
| 87 | CL- | Light | Nu- | gacatccagatgacccagtctccatcctccctgtctgcatctgtaggagacagagtcaccatcactt |
| | 82658 | chain | cleic | gccgggcaagtcagggcattagaaatgatttaagctggtatcagcagaaaccagggaaagcccctaa |
| | | vari- | acid | gcgcctgatctatgctgcatccagtttgcaaagtggggtcccatcaaggttcagcggcagtggatct |
| | | able | se- | gggacagaattcactctcacaatcagcagcctgcagcctgaagattttgcaacttattactgtctac |
| | | region | quence | agcataatggttacccattcactttcggccctgggaccaaagtggatatcaaac |
| | | | of V_{L} | |
| | | | of | |
| | | | CL- | |
| | | | 82658 | |
| 88 | CL- | Full | Amino | DIQMTQSPSSLSASVGDRVTITCRASQGIRNDLSWYQQKPGKAPKRLIYAASSLQSGVPSRFSGS |
| | 82658 | light | acid | GSGTEFTLTISSLQPEDFATYYCLQHNGYPFTFGPGTKVDIKRTVAAPSVFIFPPSDEQLKSGTASV |
| | | chain | se- | VCLLNNFYPREAKVQWKVDNALQSGNSQESVTEQDSKDSTYSLSSTLTLSKADYEKHKVYACEVT |
| | | se- | quence | HQGLSSPVTKSFNRGEC |
| | | quence | of CL- | |
| | | | 82658 | |
| | | | light | |
| | | | chain | |
| 89 | CL- | Full | Nu- | gacatccagatgacccagtctccatcctccctgtctgcatctgtaggagacagagtcaccatcactt |
| | 82658 | light | cleic | gccgggcaagtcagggcattagaaatgatttaagctggtatcagcagaaaccagggaaagcccctaa |
| | | chain | acid | gcgcctgatctatgctgcatccagtttgcaaagtggggtcccatcaaggttcagcggcagtggatct |
| | | se- | se- | gggacagaattcactctcacaatcagcagcctgcagcctgaagattttgcaacttattactgtctac |
| | | quence | quence | agcataatggttacccattcactttcggccctgggaccaaagtggatatcaaacgtacggtggccgc |
| | | | of CL- | tccctccgtgttcatcttcccaccttccgacgagcagctgaagtccggcaccgcttctgtcgtgtgc |
| | | | 82658 | ctgctgaacaacttctacccccgcgaggccaaggtgcagtggaaggtggacaacgccctgcagtccg |
| | | | light | gcaactcccaggaatccgtgaccgagcaggactccaaggacagcacctactccctgtcctccaccct |
| | | | chain | gaccctgtccaaggccgactacgagaagcacaaggtgtacgcctgcgaagtgacccaccagggcctg |
| | | | | tctagccccgtgaccaagtctttcaaccggggcgagtgt |
| 90 | CL- | CDRH1 | Amino | GFTFSGSA |
| | 83083 | (IMGT) | acid | |
| | & CL- | | se- | |
| | 83083- | | quence | |
| | 2 | | of | |
| | | | CDRH1 | |
| | | | of CL- | |
| | | | 83083 | |
| | | | & CL- | |
| | | | 83083- | |
| | | | 2 | |
| | | | using | |
| | | | IMGT | |
| 91 | CL- | CDRH2 | Amino | IRSKANKYAT |
| | 83083 | (IMGT) | acid | |
| | & CL- | | se- | |
| | 83083- | | quence | |
| | 2 | | of | |
| | | | CDRH2 | |
| | | | of CL- | |
| | | | 83083 | |
| | | | & CL- | |
| | | | 83083- | |
| | | | 2 | |
| | | | using | |
| | | | IMGT | |
| 92 | CL- | CDRH3 | Amino | TWELSNYDILTGFYYGMDV |
| | 83083 | (IMGT) | acid | |
| | & CL- | | se- | |
| | 83083- | | quence | |
| | 2 | | of | |
| | | | CDRH3 | |
| | | | of CL- | |
| | | | 83083 | |
| | | | & CL- | |
| | | | 83083- | |
| | | | 2 | |
| | | | using | |
| | | | IMGT | |
| 93 | CL- | CDRH1 | Amino | GSAMH |
| | 83083 | (KABAT) | acid | |
| | & CL- | | se- | |
| | 83083- | | quence | |
| | 2 | | of | |
| | | | CDRH1 | |
| | | | of CL- | |
| | | | 83083 | |
| | | | & CL- | |
| | | | 83083- | |
| | | | 2 | |
| | | | using | |
| | | | Kabat | |
| 94 | CL- | CDRH2 | Amino | RIRSKANKYATAYAASVKG |
| | 83083 | (KABAT) | acid | |
| | & CL- | | se- | |
| | 83083- | | quence | |
| | 2 | | of | |
| | | | CDRH2 | |
| | | | of CL- | |
| | | | 83083 | |
| | | | & CL- | |
| | | | 83083- | |
| | | | 2 | |
| | | | using | |
| | | | Kabat | |
| 95 | CL- | CDRH3 | Amino | ELSNYDILTGFYYGMDV |
| | 83083 | (KABAT) | acid | |
| | & CL- | | se- | |
| | 83083- | | quence | |
| | 2 | | of | |
| | | | CDRH3 | |
| | | | of CL- | |
| | | | 83083 | |
| | | | & CL- | |
| | | | 83083- | |
| | | | 2 | |
| | | | using | |
| | | | Kabat | |
| 96 | CL- | Heavy | Amino | EVQLVESGGGLVQPGGSLKLSCAASGFTFSGSAMHWVRQASGKGLEWVGRIRSKANKYATAYAA |
| | 83083 | chain | acid | SVKGRFTISRDDSKNTAYVQMKSLKTEDTAVYYCTWELSNYDILTGFYYGMDVWGQGTTVTVSS |
| | & CL- | vari- | se- | |
| | 83083- | able | quence | |
| | 2 | region | of V_{H} | |
| | | | of CL- | |
| | | | 83083 | |
| | | | & CL- | |
| | | | 83083- | |
| | | | 2 | |
| 97 | CL- | Heavy | Nu- | gaggtgcagctggtggagtccgggggaggcttggtccagccgggggggtccctgaaactctcctgtg |
| | 83083 | chain | cleic | cagcctctgggttcaccttcagtggatctgctatgcactgggtccgccaggcttccgggaaagggct |
| | & CL- | vari- | acid | ggagtgggttggccgtattagaagtaaagctaacaaatatgcgacagcatatgctgcgtcggtgaaa |
| | 83083- | able | se- | ggcaggttcaccatctccagagatgattcaaagaatacggcgtatgtgcaaatgaagagcctgaaaa |
| | 2 | region | quence | ccgaggacacggccgtgtattactgtacttgggaattgtcgaattacgatattttgactggtttcta |
| | | | of V_{H} | ctacggtatggacgtctggggccaagggaccacggtcaccgtctcctcag |
| | | | of CL- | |
| | | | 83083 | |
| | | | & CL- | |
| | | | 83083- | |
| | | | 2 | |
| 98 | CL- | Full | Amino | EVQLVESGGGLVQPGGSLKLSCAASGFTFSGSAMHWVRQASGKGLEWVGRIRSKANKYATAYAA |
| | 83083 | heavy | acid | SVKGRFTISRDDSKNTAYVQMKSLKTEDTAVYYCTWELSNYDILTGFYYGMDVWGQGTTVTVSS |
| | & CL- | chain | se- | ASTKGPSVFPLAPCSRSTSESTAALGCLVKDYFPEPVTVSWNSGALTSGVHTFPAVLQSSGLYSLS |
| | 83083- | se- | quence | SVVTVPSSSLGTKTYTCNVDHKPSNTKVDKRVESKYGPPCPPCPAPEFEGGPSVFLFPPKPKDTLM |
| | 2 | quence | of CL- | ISRTPEVTCVVVDVSQEDPEVQFNWYVDGVEVHNAKTKPREEQFNSTYRVVSVLTVLHQDWLNG |
| | | | 83083 | KEYKCKVSNKGLPSSIEKTISKAKGQPREPQVYTLPPSQEEMTKNQVSLTCLVKGFYPSDIAVEWE |
| | | | & CL- | SNGQPENNYKTTPPVLDSDGSFFLYSRLTVDKSRWQEGNVFSCSVMHEALHNHYTQKSLSLSLG |
| | | | 83083- | K |
| | | | 2 | |
| | | | heavy | |
| | | | chain | |
| 99 | CL- | Full | Nu- | gaggtgcagctggtggagtccgggggaggcttggtccagccgggggggtccctgaaactctcctgtg |
| | 83083 | heavy | cleic | cagcctctgggttcaccttcagtggatctgctatgcactgggtccgccaggcttccgggaaagggct |
| | & CL- | chain | acid | ggagtgggttggccgtattagaagtaaagctaacaaatatgcgacagcatatgctgcgtcggtgaaa |
| | 83083- | se- | se- | ggcaggttcaccatctccagagatgattcaaagaatacggcgtatgtgcaaatgaagagcctgaaaa |
| | 2 | quence | quence | ccgaggacacggccgtgtattactgtacttgggaattgtcgaattacgatattttgactggtttcta |
| | | | of CL- | ctacggtatggacgtctggggccaagggaccacggtcaccgtctcctcagccagcaccaagggccct |
| | | | 83083 | tccgtgttccccctggccccttgcagcaggagcacctccgaatccacagctgccctgggctgtctgg |
| | | | & CL- | tgaaggactactttcccgagcccgtgaccgtgagctggaacagcggcgctctgacatccggcgtcca |
| | | | 83083- | cacctttcctgccgtcctgcagtcctccggcctctactccctgtcctccgtggtgaccgtgcctagc |
| | | | 2 | tcctccctcggcaccaagacctacacctgtaacgtggaccacaaaccctccaacaccaaggtggaca |
| | | | heavy | aacgggtcgagagcaagtacggccctccctgccctccttgtcctgcccccgagttcgaaggcggacc |
| | | | chain | cagcgtgttcctgttccctcctaagcccaaggacaccctcatgatcagccggacacccgaggtgacc |
| | | | | tgcgtggtggtggatgtgagccaggaggaccctgaggtccagttcaactggtatgtggatggcgtgg |
| | | | | aggtgcacaacgccaagacaaagccccgggaagagcagttcaactccacctacagggtggtcagcgt |
| | | | | gctgaccgtgctgcatcaggactggctgaacggcaaggagtacaagtgcaaggtcagcaataaggga |
| | | | | ctgcccagcagcatcgagaagaccatctccaaggctaaaggccagccccgggaacctcaggtgtaca |
| | | | | ccctgcctcccagccaggaggagatgaccaagaaccaggtgagcctgacctgcctggtgaagggatt |
| | | | | ctacccttccgacatcgccgtggagtgggagtccaacggccagcccgagaacaattataagaccacc |
| | | | | cctcccgtcctcgacagcgacggatccttctttctgtactccaggctgaccgtggataagtccaggt |
| | | | | ggcaggaaggcaacgtgttcagctgctccgtgatgcacgaggccctgcacaatcactacacccagaa |
| | | | | gtccctgagcctgtccctgggaaag |
| 100 | CL- | CDRL1 | Amino | SNNVGNLG |
| | 83083 | (IMGT) | acid | |
| | & CL- | | se- | |
| | 83083- | | quence | |
| | 2 | | of | |
| | | | CDRL1 | |
| | | | of CL- | |
| | | | 83083 | |
| | | | & CL- | |
| | | | 83083- | |
| | | | 2 | |
| | | | using | |
| | | | IMGT | |
| 101 | CL- | CDRL2 | Amino | RNN |
| | 83083 | (IMGT) | acid | |
| | & CL- | | se- | |
| | 83083- | | quence | |
| | 2 | | of | |
| | | | CDRL2 | |
| | | | of CL- | |
| | | | 83083 | |
| | | | & CL- | |
| | | | 83083- | |
| | | | 2 | |
| | | | using | |
| | | | IMGT | |
| 102 | CL- | CDRL3 | Amino | SAWDSSLSAWV |
| | 83083 | (IMGT) | acid | |
| | & CL- | | se- | |
| | 83083- | | quence | |
| | 2 | | of | |
| | | | CDRL3 | |
| | | | of CL- | |
| | | | 83083 | |
| | | | & CL- | |
| | | | 83083- | |
| | | | 2 | |
| | | | using | |
| | | | IMGT | |
| 103 | CL- | CDRL1 | Amino | TGNSNNVGNLGAA |
| | 83083 | (KABAT) | acid | |
| | & CL- | | se- | |
| | 83083- | | quence | |
| | 2 | | of | |
| | | | CDRL1 | |
| | | | of CL- | |
| | | | 83083 | |
| | | | & CL- | |
| | | | 83083- | |
| | | | 2 | |
| | | | using | |
| | | | KABAT | |
| 104 | CL- | CDRL2 | Amino | RNNNRPS |
| | 83083 | (KABAT) | acid | |
| | & CL- | | se- | |
| | 83083- | | quence | |
| | 2 | | of | |
| | | | CDRL2 | |
| | | | of CL- | |
| | | | 83083 | |
| | | | & CL- | |
| | | | 83083- | |
| | | | 2 | |
| | | | using | |
| | | | KABAT | |
| 105 | CL- | CDRL3 | Amino | SAWDSSLSAWV |
| | 83083 | (KABAT) | acid | |
| | & CL- | | se- | |
| | 83083- | | quence | |
| | 2 | | of | |
| | | | CDRL3 | |
| | | | of CL- | |
| | | | 83083 | |
| | | | & CL- | |
| | | | 83083- | |
| | | | 2 | |
| | | | using | |
| | | | KABAT | |
| 106 | CL- | Light | Amino | QAGLTQPPSVSKGLRQTATLTCTGNSNNVGNLGAAWLQQHQGHPPKLLSYRNNNRPSGISERFF |
| | 83083 | chain | acid | ASRSGNTASLTITGLQPEDEADYYCSAWDSSLSAWVFGGGTKLTVL |
| | & CL- | vari- | se- | |
| | 83083- | able | quence | |
| | 2 | region | of V_{L} | |
| | | | of CL- | |
| | | | 83083 | |
| | | | & CL- | |
| | | | 83083- | |
| | | | 2 | |
| 107 | CL- | Light | Nu- | caggcagggctgactcagccaccctcggtgtccaagggcttgagacagaccgccacactcacctgca |
| | 83083 | chain | cleic | ctgggaacagcaacaatgttggcaacctaggagcagcttggctgcagcagcaccagggccaccctcc |
| | & CL- | vari- | acid | caaactcctatcctacaggaataataaccggccctcagggatctcagagagattctttgcatccagg |
| | 83083- | able | se- | tcaggaaacacagcctccctgaccattactggactccagcctgaggacgaggctgactattactgct |
| | 2 | region | quence | cagcatgggacagcagcctcagtgcttgggtgttcggcggagggaccaagctgaccgtcctag |
| | | | of V_{L} | |
| | | | of CL- | |
| | | | 83083 | |
| | | | & CL- | |
| | | | 83083- | |
| | | | 2 | |
| 108 | CL- | Full | Amino | QAGLTQPPSVSKGLRQTATLTCTGNSNNVGNLGAAWLQQHQGHPPKLLSYRNNNRPSGISERFF |
| | 83083 | light | acid | ASRSGNTASLTITGLQPEDEADYYCSAWDSSLSAWVFGGGTKLTVLGQPKAAPSVTLFPPSSEEL |
| | | chain | se- | QANKATLVCLISDFSPGAVTVAWKADSSPVKAGVETTTPSKQSNNKYAASSYLSLTPEQWKSHKS |
| | | se- | quence | YSCQVTHEGSTVKKTVAPTECS |
| | | quence | of CL- | |
| | | | 83083 | |
| | | | light | |
| | | | chain | |
| 109 | CL- | Full | Nu- | caggcagggctgactcagccaccctcggtgtccaagggcttgagacagaccgccacactcacctgca |
| | 83083 | light | cleic | ctgggaacagcaacaatgttggcaacctaggagcagcttggctgcagcagcaccagggccaccctcc |
| | | chain | acid | caaactcctatcctacaggaataataaccggccctcagggatctcagagagattctttgcatccagg |
| | | se- | se- | tcaggaaacacagcctccctgaccattactggactccagcctgaggacgaggctgactattactgct |
| | | quence | quence | cagcatgggacagcagcctcagtgcttgggtgttcggcggagggaccaagctgaccgtcctaggtca |
| | | | of CL- | gcccaaggctgccccctcggtcactctgttcccaccctcctctgaggagcttcaagccaacaaggcc |
| | | | 83083 | acactggtgtgtctcataagtgacttctccccgggagccgtgacagtggcctggaaggcagatagca |
| | | | light | gccccgtcaaggcgggagtggagaccaccacaccctccaaacaaagcaacaacaagtacgcggccag |
| | | | chain | cagctacctgagcctgacgcctgagcagtggaagtcccacaaaagctacagctgccaggtcacgcat |
| | | | | gaagggagcaccgtgaagaagacagtggcccctacagaatgttca |
| 110 | CL- | Full | Amino | QAGLTQPPSVSKGLRQTATLTCTGNSNNVGNLGAAWLQQHQGHPPKLLSYRNNNRPSGISERFF |
| | 83083- | light | acid | ASRSGNTASLTITGLQPEDEADYYCSAWDSSLSAWVFGGGTKLTVLGQPKAAPSVTLFPPSSEEL |
| | 2 | chain | se- | QANKATLVCLISDFYPGAVTVAWKADSSPVKAGVETTTPSKQSNNKYAASSYLSLTPEQWKSHKS |
| | | se- | quence | YSCQVTHEGSTVEKTVAPTECS |
| | | quence | of CL- | |
| | | | 83083- | |
| | | | 2 | |
| | | | light | |
| | | | chain | |
| 111 | CL- | Full | Nu- | caagctggattgacacagcctcctagcgtgtccaagggcctgagacagaccgctacactgacctgca |
| | 83083- | light | cleic | ccggcaactctaacaacgtgggcaatctgggcgctgcctggttgcagcagcatcagggacatcctcc |
| | 2 | chain | acid | aaagctgctgtcctaccggaacaacaaccggccttccggcatctccgagcggttcttcgcttctaga |
| | | se- | se- | tccggcaataccgccagcctgaccatcactggactgcagcctgaggacgaggccgactactactgtt |
| | | quence | quence | ctgcctgggactcctctctgtccgcctgggttttcggcggaggcacaaaactgacagtgctgggcca |
| | | | of CL- | gcctaaggccgctccttccgttacactgttccctccatcctccgaggaactgcaggccaacaaggct |
| | | | 83083- | accctcgtgtgcctgatctccgacttttaccctggcgctgtgaccgtggcctggaaggctgatagtt |
| | | | 2 | ctcctgtgaaggccggcgtggaaaccaccacaccttccaagcagtccaacaacaaatacgccgcctc |
| | | | light | ctcctacctgtctctgacccctgaacagtggaagtcccacaagtcctacagctgccaagtgacccac |
| | | | chain | gagggctccaccgtggaaaagacagtggctcctaccgagtgctcc |
| 112 | CL- | Heavy | Amino | QVQLVESGGGVVQPGRSLRLSCAASGFTFYSYGMHWVRQAPGKGLEWVAVIWYGGTNKYYADS |
| | 82583 | chain | acid | VKGRFTISRDNSKNTLYLQMNSLTAEDTAVYYCARGQLERRRGGLNIWGQGTMVTVSS |
| | | vari- | se- | |
| | | able | quence | |
| | | region | of V_{H} | |
| | | | of CL- | |
| | | | 82583 | |
| 113 | CL- | Heavy | Nu- | caggtgcagctggtggagtctgggggaggcgtggtccagcctgggaggtccctgagactctcctgtg |
| | 82583 | chain | cleic | cagcgtctggattcaccttctatagctatggcatgcactgggtccgccaggctccaggcaaggggct |
| | | vari- | acid | ggagtgggtggcagttatatggtatggtggaactaataaatactatgcagactccgtgaagggccga |
| | | able | se- | ttcaccatctccagagacaattccaagaacacgctgtatctgcaaatgaacagcctgacagccgaag |
| | | region | quence | acacggctgtgtattactgtgcgagagggcaactggaacgccgacggggcggtttgaatatctgggg |
| | | | of V_{H} | ccaagggacaatggtcaccgtctcttcag |
| | | | of CL- | |
| | | | 82583 | |
| 114 | CL- | Full | Amino | QVQLVESGGGVVQPGRSLRLSCAASGFTFYSYGMHWVRQAPGKGLEWVAVIWYGGTNKYYADS |
| | 82583 | heavy | acid | VKGRFTISRDNSKNTLYLQMNSLTAEDTAVYYCARGQLERRRGGLNIWGQGTMVTVSSASTKGP |
| | | chain | se- | SVFPLAPCSRSTSESTAALGCLVKDYFPEPVTVSWNSGALTSGVHTFPAVLQSSGLYSLSSVVTVP |
| | | se- | quence | SSSLGTKTYTCNVDHKPSNTKVDKRVESKYGPPCPPCPAPEFEGGPSVFLFPPKPKDTLMISRTPE |
| | | quence | of CL- | VTCVVVDVSQEDPEVQFNWYVDGVEVHNAKTKPREEQFNSTYRVVSVLTVLHQDWLNGKEYKC |
| | | | 82583 | KVSNKGLPSSIEKTISKAKGQPREPQVYTLPPSQEEMTKNQVSLTCLVKGFYPSDIAVEWESNGQP |
| | | | heavy | ENNYKTTPPVLDSDGSFFLYSRLTVDKSRWQEGNVFSCSVMHEALHNHYTQKSLSLSLGK |
| | | | chain | |
| 115 | CL- | Full | Nu- | caggtgcagctggtggagtctgggggaggcgtggtccagcctgggaggtccctgagactctcctgtg |
| | 82583 | heavy | cleic | cagcgtctggattcaccttctatagctatggcatgcactgggtccgccaggctccaggcaaggggct |
| | | chain | acid | ggagtgggtggcagttatatggtatggtggaactaataaatactatgcagactccgtgaagggccga |
| | | se- | se- | ttcaccatctccagagacaattccaagaacacgctgtatctgcaaatgaacagcctgacagccgaag |
| | | quence | quence | acacggctgtgtattactgtgcgagagggcaactggaacgccgacggggcggtttgaatatctgggg |
| | | | of CL- | ccaagggacaatggtcaccgtctcttcagccagcaccaagggcccttccgtgttccccctggcccct |
| | | | 82583 | tgcagcaggagcacctccgaatccacagctgccctgggctgtctggtgaaggactactttcccgagc |
| | | | heavy | ccgtgaccgtgagctggaacagcggcgctctgacatccggcgtccacacctttcctgccgtcctgca |
| | | | chain | gtcctccggcctctactccctgtcctccgtggtgaccgtgcctagctcctccctcggcaccaagacc |
| | | | | tacacctgtaacgtggaccacaaaccctccaacaccaaggtggacaaacgggtcgagagcaagtacg |
| | | | | gccctccctgccctccttgtcctgcccccgagttcgaaggcggacccagcgtgttcctgttccctcc |
| | | | | taagcccaaggacaccctcatgatcagccggacacccgaggtgacctgcgtggtggtggatgtgagc |
| | | | | caggaggaccctgaggtccagttcaactggtatgtggatggcgtggaggtgcacaacgccaagacaa |
| | | | | agccccgggaagagcagttcaactccacctacagggtggtcagcgtgctgaccgtgctgcatcagga |
| | | | | ctggctgaacggcaaggagtacaagtgcaaggtcagcaataagggactgcccagcagcatcgagaag |
| | | | | accatctccaaggctaaaggccagccccgggaacctcaggtgtacaccctgcctcccagccaggagg |
| | | | | agatgaccaagaaccaggtgagcctgacctgcctggtgaagggattctacccttccgacatcgccgt |
| | | | | ggagtgggagtccaacggccagcccgagaacaattataagaccacccctcccgtcctcgacagcgac |
| | | | | ggatccttctttctgtactccaggctgaccgtggataagtccaggtggcaggaaggcaacgtgttca |
| | | | | gctgctccgtgatgcacgaggccctgcacaatcactacacccagaagtccctgagcctgtccctggg |
| | | | | aaag |
| 116 | CL- | Light | Amino | DIQMTQSPSSLSASVGDRVTITCRASQGVRDDLGWYQQKPGKAPKRLIYAASSLQSGVPSRFSGS |
| | 82583 | chain | acid | GSGTEFTLTISSLQPEDFATYYCLQHNSYPFTFGPGTKVDIK |
| | | vari- | se- | |
| | | able | quence | |
| | | region | of V_{L} | |
| | | | of CL- | |
| | | | 82583 | |
| 117 | CL- | Light | Nu- | gacatccagatgacccagtctccatcctccctgtctgcatctgtaggagacagagtcaccatcactt |
| | 82583 | chain | cleic | gccgggcaagtcagggcgttagagatgatttaggttggtatcagcagaaaccagggaaagcccctaa |
| | | vari- | acid | gcgcctgatctatgctgcatccagtttgcaaagtggggtcccatcaaggttcagcggcagtggatct |
| | | able | se- | gggacagaattcactctcacaatcagcagcctgcagcctgaagattttgcaacttattactgtctac |
| | | region | quence | agcataatagttatccattcactttcggccctgggaccaaagtggatatcaaac |
| | | | of V_{L} | |
| | | | of CL- | |
| | | | 82583 | |
| 118 | CL- | Full | Amino | DIQMTQSPSSLSASVGDRVTITCRASQGVRDDLGWYQQKPGKAPKRLIYAASSLQSGVPSRFSGS |
| | 82583 | light | acid | GSGTEFTLTISSLQPEDFATYYCLQHNSYPFTFGPGTKVDIKRTVAAPSVFIFPPSDEQLKSGTASV |
| | | chain | se- | VCLLNNFYPREAKVQWKVDNALQSGNSQESVTEQDSKDSTYSLSSTLTLSKADYEKHKVYACEVT |
| | | se- | quence | HQGLSSPVTKSFNRGEC |
| | | quence | of CL- | |
| | | | 82583 | |
| | | | light | |
| | | | chain | |
| 119 | CL- | Full | Nu- | gacatccagatgacccagtctccatcctccctgtctgcatctgtaggagacagagtcaccatcactt |
| | 82583 | light | cleic | gccgggcaagtcagggcgttagagatgatttaggttggtatcagcagaaaccagggaaagcccctaa |
| | | chain | acid | gcgcctgatctatgctgcatccagtttgcaaagtggggtcccatcaaggttcagcggcagtggatct |
| | | se- | se- | gggacagaattcactctcacaatcagcagcctgcagcctgaagattttgcaacttattactgtctac |
| | | quence | quence | agcataatagttatccattcactttcggccctgggaccaaagtggatatcaaacgtacggtggccgc |
| | | | of CL- | tccctccgtgttcatcttcccaccttccgacgagcagctgaagtccggcaccgcttctgtcgtgtgc |
| | | | 82583 | ctgctgaacaacttctacccccgcgaggccaaggtgcagtggaaggtggacaacgccctgcagtccg |
| | | | light | gcaactcccaggaatccgtgaccgagcaggactccaaggacagcacctactccctgtcctccaccct |
| | | | chain | gaccctgtccaaggccgactacgagaagcacaaggtgtacgcctgcgaagtgacccaccagggcctg |
| | | | | tctagccccgtgaccaagtctttcaaccggggcgagtgt |
| 120 | CL- | Heavy | Amino | QVQLVQSGAEVKKPGASVKVSCRASGYTFTNYDINWVRQATGQGLEWMGWMNPNSGNTGYA |
| | 82580 | chain | acid | QKFQGRVTMTRNTSISAAYMELSSLRSEDTAVYYCARGRGYYGSGSRYRGYYYYGMDVWGQGT |
| | | vari- | se- | TVTVSS |
| | | able | quence | |
| | | region | of V_{H} | |
| | | | of CL- | |
| | | | 82580 | |
| 121 | CL- | Heavy | Nu- | caggtgcagctggtgcagtctggggctgaggtgaagaagcctggggcctcagtgaaggtctcctgca |
| | 82580 | chain | cleic | gggcttctggatacaccttcaccaattatgatatcaactgggtgcgacaggccactggacaagggct |
| | | vari- | acid | tgagtggatgggatggatgaaccctaacagtggtaacacaggctatgcacagaagttccagggcaga |
| | | able | se- | gtcaccatgaccaggaacacctccataagcgcagcctacatggagctgagcagcctgagatctgaag |
| | | region | quence | acacggccgtgtattactgtgcgagaggccgggggtactatggttcggggagccgttataggggcta |
| | | | of V_{H} | ctactactacggtatggacgtctggggccaagggaccacggtcaccgtctcctcag |
| | | | of CL- | |
| | | | 82580 | |
| 122 | CL- | Full | Amino | QVQLVQSGAEVKKPGASVKVSCRASGYTFTNYDINWVRQATGQGLEWMGWMNPNSGNTGYA |
| | 82580 | heavy | acid | QKFQGRVTMTRNTSISAAYMELSSLRSEDTAVYYCARGRGYYGSGSRYRGYYYYGMDVWGQGT |
| | | chain | se- | TVTVSSASTKGPSVFPLAPCSRSTSESTAALGCLVKDYFPEPVTVSWNSGALTSGVHTFPAVLQSS |
| | | se- | quence | GLYSLSSVVTVPSSSLGTKTYTCNVDHKPSNTKVDKRVESKYGPPCPPCPAPEFEGGPSVFLFPPK |
| | | quence | of CL- | PKDTLMISRTPEVTCVVVDVSQEDPEVQFNWYVDGVEVHNAKTKPREEQFNSTYRVVSVLTVLH |
| | | | 82580 | QDWLNGKEYKCKVSNKGLPSSIEKTISKAKGQPREPQVYTLPPSQEEMTKNQVSLTCLVKGFYPS |
| | | | heavy | DIAVEWESNGQPENNYKTTPPVLDSDGSFFLYSRLTVDKSRWQEGNVFSCSVMHEALHNHYTQK |
| | | | chain | SLSLSLGK |
| 123 | CL- | Full | Nu- | caggtgcagctggtgcagtctggggctgaggtgaagaagcctggggcctcagtgaaggtctcctgca |
| | 82580 | heavy | cleic | gggcttctggatacaccttcaccaattatgatatcaactgggtgcgacaggccactggacaagggct |
| | | chain | acid | tgagtggatgggatggatgaaccctaacagtggtaacacaggctatgcacagaagttccagggcaga |
| | | se- | se- | gtcaccatgaccaggaacacctccataagcgcagcctacatggagctgagcagcctgagatctgaag |
| | | quence | quence | acacggccgtgtattactgtgcgagaggccgggggtactatggttcggggagccgttataggggcta |
| | | | of CL- | ctactactacggtatggacgtctggggccaagggaccacggtcaccgtctcctcagccagcaccaag |
| | | | 82580 | ggcccttccgtgttccccctggccccttgcagcaggagcacctccgaatccacagctgccctgggct |
| | | | heavy | gtctggtgaaggactactttcccgagcccgtgaccgtgagctggaacagcggcgctctgacatccgg |
| | | | chain | cgtccacacctttcctgccgtcctgcagtcctccggcctctactccctgtcctccgtggtgaccgtg |
| | | | | cctagctcctccctcggcaccaagacctacacctgtaacgtggaccacaaaccctccaacaccaagg |
| | | | | tggacaaacgggtcgagagcaagtacggccctccctgccctccttgtcctgcccccgagttcgaagg |
| | | | | cggacccagcgtgttcctgttccctcctaagcccaaggacaccctcatgatcagccggacacccgag |
| | | | | gtgacctgcgtggtggtggatgtgagccaggaggaccctgaggtccagttcaactggtatgtggatg |
| | | | | gcgtggaggtgcacaacgccaagacaaagccccgggaagagcagttcaactccacctacagggtggt |
| | | | | cagcgtgctgaccgtgctgcatcaggactggctgaacggcaaggagtacaagtgcaaggtcagcaat |
| | | | | aagggactgcccagcagcatcgagaagaccatctccaaggctaaaggccagccccgggaacctcagg |
| | | | | tgtacaccctgcctcccagccaggaggagatgaccaagaaccaggtgagcctgacctgcctggtgaa |
| | | | | gggattctacccttccgacatcgccgtggagtgggagtccaacggccagcccgagaacaattataag |
| | | | | accacccctcccgtcctcgacagcgacggatccttctttctgtactccaggctgaccgtggataagt |
| | | | | ccaggtggcaggaaggcaacgtgttcagctgctccgtgatgcacgaggccctgcacaatcactacac |
| | | | | ccagaagtccctgagcctgtccctgggaaag |
| 124 | CL- | Light | Amino | DIQMTQSPSTLSASVGDRVTITCRASQSISSWLAWYQQKPGKAPKVLIYKASSLESGVPSRFSGS |
| | 82580 | chain | acid | GSGTEFTLTISSLQPDDFATYYCQQYNSYSWTFGQGTKVEIK |
| | | vari- | se- | |
| | | able | quence | |
| | | region | of V_{L} | |
| | | | of CL- | |
| | | | 82580 | |
| 125 | CL- | Light | Nu- | gacatccagatgacccagtctccttccaccctgtctgcatctgtaggagacagagtcaccatcactt |
| | 82580 | chain | cleic | gccgggccagtcagagtattagtagctggttggcctggtatcagcagaaaccagggaaagcccctaa |
| | | vari- | acid | ggtcctgatctataaggcgtctagtttagaaagtggggtcccatcaaggttcagcggcagtggatct |
| | | able | se- | gggacagaattcactctcaccatcagcagcctgcagcctgatgattttgcaacttattactgccaac |
| | | region | quence | agtataatagttattcgtggacgttcggccaagggaccaaggtggaaatcaaac |
| | | | of V_{L} | |
| | | | of CL- | |
| | | | 82580 | |
| 126 | CL- | Full | Amino | DIQMTQSPSTLSASVGDRVTITCRASQSISSWLAWYQQKPGKAPKVLIYKASSLESGVPSRFSGS |
| | 82580 | light | acid | GSGTEFTLTISSLQPDDFATYYCQQYNSYSWTFGQGTKVEIKRTVAAPSVFIFPPSDEQLKSGTAS |
| | | chain | se- | WVCLLNNFYPREAKVQWKVDNALQSGNSQESVTEQDSKDSTYSLSSTLTLSKADYEKHKVYACEV |
| | | se- | quence | THQGLSSPVTKSFNRGEC |
| | | quence | of CL- | |
| | | | 82580 | |
| | | | light | |
| | | | chain | |
| 127 | CL- | Full | Nu- | gacatccagatgacccagtctccttccaccctgtctgcatctgtaggagacagagtcaccatcactt |
| | 82580 | light | cleic | gccgggccagtcagagtattagtagctggttggcctggtatcagcagaaaccagggaaagcccctaa |
| | | chain | acid | ggtcctgatctataaggcgtctagtttagaaagtggggtcccatcaaggttcagcggcagtggatct |
| | | se- | se- | gggacagaattcactctcaccatcagcagcctgcagcctgatgattttgcaacttattactgccaac |
| | | quence | quence | agtataatagttattcgtggacgttcggccaagggaccaaggtggaaatcaaacgtacggtggccgc |
| | | | of CL- | tccctccgtgttcatcttcccaccttccgacgagcagctgaagtccggcaccgcttctgtcgtgtgc |
| | | | 82580 | ctgctgaacaacttctacccccgcgaggccaaggtgcagtggaaggtggacaacgccctgcagtccg |
| | | | light | gcaactcccaggaatccgtgaccgagcaggactccaaggacagcacctactccctgtcctccaccct |
| | | | chain | gaccctgtccaaggccgactacgagaagcacaaggtgtacgcctgcgaagtgacccaccagggcctg |
| | | | | tctagccccgtgaccaagtctttcaaccggggcgagtgt |
| 128 | CL- | Heavy | Amino | QVQLQESGPGLVKPSQTLSLTCTVSGGSISSGGYYWTWIRQLPGKGLEWIGYMSYSGNTF |
| | 82577 | chain | acid | YSPSLKSRFTISGDTSENQFSLKVNSVTSADTAIYYCARDRRIPMLRGIKGYYYGMDVWGQGTTV |
| | | vari- | se | TVSS |
| | | able | quence | |
| | | region | of V_{H} | |
| | | | of CL- | |
| | | | 82577 | |
| 129 | CL- | Heavy | Nu- | caggtgcagctgcaggagtcgggcccaggactggtgaagccttcacagaccctgtccctcacctgca |
| | 82577 | chain | cleic | ctgtctctggtggctccatcagcagtggtggatactactggacctggatccgccagctcccagggaa |
| | | vari- | acid | gggcctggagtggattgggtacatgtcatacagtgggaacaccttctacagcccgtccctcaagagt |
| | | able | se- | cgatttacaatatcaggagacacgtctgagaaccagttctccctgaaggtgaactctgtgacttccg |
| | | region | quence | cggacacggccatttattactgtgcgagagatcgacgtattcctatgcttcgcggaattaaaggcta |
| | | | of V_{H} | ctactacggtatggacgtctggggccaagggaccacggtcaccgtctcctcag |
| | | | of CL- | |
| | | | 82577 | |
| 130 | CL- | Full | Amino | QVQLQESGPGLVKPSQTLSLTCTVSGGSISSGGYYWTWIRQLPGKGLEWIGYMSYSGNTFYSPSL |
| | 82577 | heavy | acid | KSRFTISGDTSENQFSLKVNSVTSADTAIYYCARDRRIPMLRGIKGYYYGMDVWGQGTTVTVSSA |
| | | chain | se- | STKGPSVFPLAPCSRSTSESTAALGCLVKDYFPEPVTVSWNSGALTSGVHTFPAVLQSSGLYSLSS |
| | | se- | quence | VVTVPSSSLGTKTYTCNVDHKPSNTKVDKRVESKYGPPCPPCPAPEFEGGPSVFLFPPKPKDTLMI |
| | | quence | of CL- | SRTPEVTCVVVDVSQEDPEVQFNWYVDGVEVHNAKTKPREEQFNSTYRVVSVLTVLHQDWLNG |
| | | | 82577 | KEYKCKVSNKGLPSSIEKTISKAKGQPREPQVYTLPPSQEEMTKNQVSLTCLVKGFYPSDIAVEWE |
| | | | heavy | SNGQPENNYKTTPPVLDSDGSFFLYSRLTVDKSRWQEGNVFSCSVMHEALHNHYTQKSLSLSLG |
| | | | chain | K |
| 131 | CL- | Full | Nu- | caggtgcagctgcaggagtcgggcccaggactggtgaagccttcacagaccctgtccctcacctgca |
| | 82577 | heavy | cleic | ctgtctctggtggctccatcagcagtggtggatactactggacctggatccgccagctcccagggaa |
| | | chain | acid | gggcctggagtggattgggtacatgtcatacagtgggaacaccttctacagcccgtccctcaagagt |
| | | se- | se- | cgatttacaatatcaggagacacgtctgagaaccagttctccctgaaggtgaactctgtgacttccg |
| | | quence | quence | cggacacggccatttattactgtgcgagagatcgacgtattcctatgcttcgcggaattaaaggcta |
| | | | of CL- | ctactacggtatggacgtctggggccaagggaccacggtcaccgtctcctcagccagcaccaagggc |
| | | | 82577 | ccttccgtgttccccctggccccttgcagcaggagcacctccgaatccacagctgccctgggctgtc |
| | | | heavy | tggtgaaggactactttcccgagcccgtgaccgtgagctggaacagcggcgctctgacatccggcgt |
| | | | chain | ccacacctttcctgccgtcctgcagtcctccggcctctactccctgtcctccgtggtgaccgtgcct |
| | | | | agctcctccctcggcaccaagacctacacctgtaacgtggaccacaaaccctccaacaccaaggtgg |
| | | | | acaaacgggtcgagagcaagtacggccctccctgccctccttgtcctgcccccgagttcgaaggcgg |
| | | | | acccagcgtgttcctgttccctcctaagcccaaggacaccctcatgatcagccggacacccgaggtg |
| | | | | acctgcgtggtggtggatgtgagccaggaggaccctgaggtccagttcaactggtatgtggatggcg |
| | | | | tggaggtgcacaacgccaagacaaagccccgggaagagcagttcaactccacctacagggtggtcag |
| | | | | cgtgctgaccgtgctgcatcaggactggctgaacggcaaggagtacaagtgcaaggtcagcaataag |
| | | | | ggactgcccagcagcatcgagaagaccatctccaaggctaaaggccagccccgggaacctcaggtgt |
| | | | | acaccctgcctcccagccaggaggagatgaccaagaaccaggtgagcctgacctgcctggtgaaggg |
| | | | | attctacccttccgacatcgccgtggagtgggagtccaacggccagcccgagaacaattataagacc |
| | | | | acccctcccgtcctcgacagcgacggatccttctttctgtactccaggctgaccgtggataagtcca |
| | | | | ggtggcaggaaggcaacgtgttcagctgctccgtgatgcacgaggccctgcacaatcactacaccca |
| | | | | gaagtccctgagcctgtccctgggaaag |
| 132 | CL- | Light | Amino | DIVMTQSPLSLPVIPGEPASISCRSSQSLLHSNGYNYLDWYLQKPGQSPQLLIYLGSNRASGVPDR |
| | 82577 | chain | acid | FRGSGSGTDFTLKIRRVEAEDVGVYYCMQALQTPWTFGQGTKVEIK |
| | | vari- | se- | |
| | | able | quence | |
| | | region | of V_{L} | |
| | | | of CL- | |
| | | | 82577 | |
| 133 | CL- | Light | Nu- | gatattgtgatgactcagtctccactctccctgcccgtcatccctggagagccggcctccatctcct |
| | 82577 | chain | cleic | gcaggtctagtcagagcctcctgcatagtaatggatacaactatttggattggtacctgcagaagcc |
| | | vari- | acid | agggcagtctccacagctcctgatctatttgggttctaatcgggcctccggggtccctgacaggttc |
| | | able | se- | cgtggcagtggatcaggcacagattttacactgaaaatcagaagagtggaggctgaggatgttgggg |
| | | region | quence | tttattactgcatgcaagctctacagactccgtggacgttcggccaagggaccaaggtggaaatcaa |
| | | | of V_{L} | ac |
| | | | of CL- | |
| | | | 82577 | |
| 134 | CL- | Full | Amino | DIVMTQSPLSLPVIPGEPASISCRSSQSLLHSNGYNYLDWYLQKPGQSPQLLIYLGSNRASGVPDR |
| | 82577 | light | acid | FRGSGSGTDFTLKIRRVEAEDVGVYYCMQALQTPWTFGQGTKVEIKRTVAAPSVFIFPPSDEQLK |
| | | chain | se- | SGTASVVCLLNNFYPREAKVQWKVDNALQSGNSQESVTEQDSKDSTYSLSSTLTLSKADYEKHK |
| | | se- | quence | VYACEVTHQGLSSPVTKSFNRGEC |
| | | quence | of CL- | |
| | | | 82577 | |
| | | | light | |
| | | | chain | |
| 135 | CL- | Full | Nu- | gatattgtgatgactcagtctccactctccctgcccgtcatccctggagagccggcctccatctcct |
| | 82577 | light | cleic | gcaggtctagtcagagcctcctgcatagtaatggatacaactatttggattggtacctgcagaagcc |
| | | chain | acid | agggcagtctccacagctcctgatctatttgggttctaatcgggcctccggggtccctgacaggttc |
| | | se- | se- | cgtggcagtggatcaggcacagattttacactgaaaatcagaagagtggaggctgaggatgttgggg |
| | | quence | quence | tttattactgcatgcaagctctacagactccgtggacgttcggccaagggaccaaggtggaaatcaa |
| | | | of CL- | acgtacggtggccgctccctccgtgttcatcttcccaccttccgacgagcagctgaagtccggcacc |
| | | | 82577 | gcttctgtcgtgtgcctgctgaacaacttctacccccgcgaggccaaggtgcagtggaaggtggaca |
| | | | light | acgccctgcagtccggcaactcccaggaatccgtgaccgagcaggactccaaggacagcacctactc |
| | | | chain | cctgtcctccaccctgaccctgtccaaggccgactacgagaagcacaaggtgtacgcctgcgaagtg |
| | | | | acccaccagggcctgtctagccccgtgaccaagtctttcaaccggggcgagtgt |
| 136 | CL- | Heavy | Amino | EVQLVESGGGLVQPGGSLKLSCAASGFTFSGSAMHWVRQASGKGLEWVGRIRSKANKYATAYAA |
| | 82571 | chain | acid | SVKGRFTISRDDSKNTAYVQMKSLKTEDTAVYYCTWELSNYDILTGFYYGMDVWGQGTTVTVSS |
| | | vari- | se- | |
| | | able | quence | |
| | | region | of V_{H} | |
| | | | of CL- | |
| | | | 82571 | |
| 137 | CL- | Heavy | Nu- | gaggtgcagctggtggagtccgggggaggcttggtccagccgggggggtccctgaaactctcctgtg |
| | 82571 | chain | cleic | cagcctctgggttcaccttcagtggatctgctatgcactgggtccgccaggcttccgggaaagggct |
| | | vari- | acid | ggagtgggttggccgtattagaagtaaagctaacaaatatgcgacagcatatgctgcgtcggtgaaa |
| | | able | se- | ggcaggttcaccatctccagagatgattcaaagaatacggcgtatgtgcaaatgaagagcctgaaaa |
| | | region | quence | ccgaggacacggccgtgtattactgtacttgggaattgtcgaattacgatattttgactggtttcta |
| | | | of V_{H} | ctacggtatggacgtctggggccaagggaccacggtcaccgtctcctcag |
| | | | of CL- | |
| | | | 82571 | |
| 138 | CL- | Full | Amino | EVQLVESGGGLVQPGGSLKLSCAASGFTFSGSAMHWVRQASGKGLEWVGRIRSKANKYATAYAA |
| | 82571 | heavy | acid | SVKGRFTISRDDSKNTAYVQMKSLKTEDTAVYYCTWELSNYDILTGFYYGMDVWGQGTTVTVSS |
| | | chain | se- | ASTKGPSVFPLAPCSRSTSESTAALGCLVKDYFPEPVTVSWNSGALTSGVHTFPAVLQSSGLYSLS |
| | | se- | quence | SVVTVPSSSLGTKTYTCNVDHKPSNTKVDKRVESKYGPPCPPCPAPEFEGGPSVFLFPPKPKDTLM |
| | | quence | of CL- | ISRTPEVTCVVVDVSQEDPEVQFNWYVDGVEVHNAKTKPREEQFNSTYRVVSVLTVLHQDWLNG |
| | | | 82571 | KEYKCKVSNKGLPSSIEKTISKAKGQPREPQVYTLPPSQEEMTKNQVSLTCLVKGFYPSDIAVEWE |
| | | | heavy | SNGQPENNYKTTPPVLDSDGSFFLYSRLTVDKSRWQEGNVFSCSVMHEALHNHYTQKSLSLSLG |
| | | | chain | K |
| 139 | CL- | Full | Nu- | gaggtgcagctggtggagtccgggggaggcttggtccagccgggggggtccctgaaactctcctgtg |
| | 82571 | heavy | cleic | cagcctctgggttcaccttcagtggatctgctatgcactgggtccgccaggcttccgggaaagggct |
| | | chain | acid | ggagtgggttggccgtattagaagtaaagctaacaaatatgcgacagcatatgctgcgtcggtgaaa |
| | | se- | se- | ggcaggttcaccatctccagagatgattcaaagaatacggcgtatgtgcaaatgaagagcctgaaaa |
| | | quence | quence | ccgaggacacggccgtgtattactgtacttgggaattgtcgaattacgatattttgactggtttcta |
| | | | of CL- | ctacggtatggacgtctggggccaagggaccacggtcaccgtctcctcagccagcaccaagggccct |
| | | | 82571 | tccgtgttccccctggccccttgcagcaggagcacctccgaatccacagctgccctgggctgtctgg |
| | | | heavy | tgaaggactactttcccgagcccgtgaccgtgagctggaacagcggcgctctgacatccggcgtcca |
| | | | chain | cacctttcctgccgtcctgcagtcctccggcctctactccctgtcctccgtggtgaccgtgcctagc |
| | | | | tcctccctcggcaccaagacctacacctgtaacgtggaccacaaaccctccaacaccaaggtggaca |
| | | | | aacgggtcgagagcaagtacggccctccctgccctccttgtcctgcccccgagttcgaaggcggacc |
| | | | | cagcgtgttcctgttccctcctaagcccaaggacaccctcatgatcagccggacacccgaggtgacc |
| | | | | tgcgtggtggtggatgtgagccaggaggaccctgaggtccagttcaactggtatgtggatggcgtgg |
| | | | | aggtgcacaacgccaagacaaagccccgggaagagcagttcaactccacctacagggtggtcagcgt |
| | | | | gctgaccgtgctgcatcaggactggctgaacggcaaggagtacaagtgcaaggtcagcaataaggga |
| | | | | ctgcccagcagcatcgagaagaccatctccaaggctaaaggccagccccgggaacctcaggtgtaca |
| | | | | ccctgcctcccagccaggaggagatgaccaagaaccaggtgagcctgacctgcctggtgaagggatt |
| | | | | ctacccttccgacatcgccgtggagtgggagtccaacggccagcccgagaacaattataagaccacc |
| | | | | cctcccgtcctcgacagcgacggatccttctttctgtactccaggctgaccgtggataagtccaggt |
| | | | | ggcaggaaggcaacgtgttcagctgctccgtgatgcacgaggccctgcacaatcactacacccagaa |
| | | | | gtccctgagcctgtccctgggaaag |
| 140 | CL- | Light | Amino | DIQMTQSPSTLSASVGDRVTITCRASQSISNWLAWYQQKPGKAPKVLIYKASSLESGVPSRFSGS |
| | 82571 | chain | acid | GSGTEFTLTISSLQPDDFATYYCQQYNSFSWTFGQGTKVEIK |
| | | vari- | se- | |
| | | able | quence | |
| | | region | of V_{L} | |
| | | | of CL- | |
| | | | 82571 | |
| 141 | CL- | Light | Nu- | gacatccagatgacccagtctccttccaccctgtctgcatctgtaggagacagagtcaccatcactt |
| | 82571 | chain | cleic | gccgggccagtcagagtattagtaattggttggcctggtatcagcagaaaccagggaaagcccctaa |
| | | vari- | acid | ggtcctgatctataaggcgtctagtttagaaagtggggtcccatcaaggttcagcggcagtggatct |
| | | able | se- | gggacagaattcactctcaccatcagcagcctgcagcctgatgattttgcaacttattactgccaac |
| | | region | quence | agtataatagtttttcgtggacgttcggccaagggaccaaggtggaaatcaaac |
| | | | of V_{L} | |
| | | | of CL- | |
| | | | 82571 | |
| 142 | CL- | Full | Amino | DIQMTQSPSTLSASVGDRVTITCRASQSISNWLAWYQQKPGKAPKVLIYKASSLESGVPSRFSGS |
| | 82571 | light | acid | GSGTEFTLTISSLQPDDFATYYCQQYNSFSWTFGQGTKVEIKRTVAAPSVFIFPPSDEQLKSGTAS |
| | | chain | se- | VVCLLNNFYPREAKVQWKVDNALQSGNSQESVTEQDSKDSTYSLSSTLTLSKADYEKHKVYACEV |
| | | se- | quence | THQGLSSPVTKSFNRGEC |
| | | quence | of CL- | |
| | | | 82571 | |
| | | | light | |
| | | | chain | |
| 143 | CL- | Full | Nu- | gacatccagatgacccagtctccttccaccctgtctgcatctgtaggagacagagtcaccatcactt |
| | 82571 | light | cleic | gccgggccagtcagagtattagtaattggttggcctggtatcagcagaaaccagggaaagcccctaa |
| | | chain | acid | ggtcctgatctataaggcgtctagtttagaaagtggggtcccatcaaggttcagcggcagtggatct |
| | | se- | se- | gggacagaattcactctcaccatcagcagcctgcagcctgatgattttgcaacttattactgccaac |
| | | quence | quence | agtataatagtttttcgtggacgttcggccaagggaccaaggtggaaatcaaacgtacggtggccgc |
| | | | of CL- | tccctccgtgttcatcttcccaccttccgacgagcagctgaagtccggcaccgcttctgtcgtgtgc |
| | | | 82571 | ctgctgaacaacttctacccccgcgaggccaaggtgcagtggaaggtggacaacgccctgcagtccg |
| | | | light | gcaactcccaggaatccgtgaccgagcaggactccaaggacagcacctactccctgtcctccaccct |
| | | | chain | gaccctgtccaaggccgactacgagaagcacaaggtgtacgcctgcgaagtgacccaccagggcctg |
| | | | | tctagccccgtgaccaagtctttcaaccggggcgagtgt |
| 144 | CL- | Heavy | Amino | QVQLVESGGGVVQPGRSLRLSCTASGFTFSNYAMHWVRQAPGKGLEWVAVISYDGSNKYCADS |
| | 82562 | chain | acid | VKGRFTISRDNSKNTLYLQMNSLRAEDTAVYYCARGPLARRHYYYYGMDVWGQGTTVTVSS |
| | | vari- | se- | |
| | | able | quence | |
| | | region | of V_{H} of | |
| | | | CL-82562 | |
| 145 | CL- | Heavy | Nu- | caggtgcagctggtggagtctgggggaggcgtggtccagcctgggaggtccctgagactctcctgta |
| | 82562 | chain | cleic | cagcctctggattcaccttcagtaactatgccatgcactgggtccgccaggctccaggcaagggact |
| | | vari- | acid | ggagtgggggcagttatatcatatgatggaagtaataaatactgtgcagactccgtgaagggccgat |
| | | able | se- | tcaccatctccagagacaattccaagaacacgctgtatctgcaaatgaacagcctgagagctgagga |
| | | region | quence | cacggctgtgtattactgtgcgagaggaccactggcacgacgccactactactactacggtatggac |
| | | | of V_{H} | gtctggggccaagggaccacggtcaccgtctcctcag |
| | | | of CL- | |
| | | | 82562 | |
| 146 | CL- | Full | Amino | QVQLVESGGGVVQPGRSLRLSCTASGFTFSNYAMHWVRQAPGKGLEWVAVISYDGSNKYCADS |
| | 82562 | heavy | acid | VKGRFTISRDNSKNTLYLQMNSLRAEDTAVYYCARGPLARRHYYYYGMDVWGQGTTVTVSSAST |
| | | chain | se- | KGPSVFPLAPCSRSTSESTAALGCLVKDYFPEPVTVSWNSGALTSGVHTFPAVLQSSGLYSLSSVV |
| | | se- | quence | TVPSSSLGTKTYTCNVDHKPSNTKVDKRVESKYGPPCPPCPAPEFEGGPSVFLFPPKPKDTLMISR |
| | | quence | of CL- | TPEVTCVVVDVSQEDPEVQFNWYVDGVEVHNAKTKPREEQFNSTYRVVSVLTVLHQDWLNGKE |
| | | | 82562 | YKCKVSNKGLPSSIEKTISKAKGQPREPQVYTLPPSQEEMTKNQVSLTCLVKGFYPSDIAVEWESN |
| | | | heavy | GQPENNYKTTPPVLDSDGSFFLYSRLTVDKSRWQEGNVFSCSVMHEALHNHYTQKSLSLSLGK |
| | | | chain | |
| 147 | CL- | Full | Nu- | caggtgcagctggtggagtctgggggaggcgtggtccagcctgggaggtccctgagactctcctgta |
| | 82562 | heavy | cleic | cagcctctggattcaccttcagtaactatgccatgcactgggtccgccaggctccaggcaagggact |
| | | chain | acid | ggagtgggtggcagttatatcatatgatggaagtaataaatactgtgcagactccgtgaagggccga |
| | | se- | se- | ttcaccatctccagagacaattccaagaacacgctgtatctgcaaatgaacagcctgagagctgagg |
| | | quence | quence | acacggctgtgtattactgtgcgagaggaccactggcacgacgccactactactactacggtatgga |
| | | | of CL- | cgtctggggccaagggaccacggtcaccgtctcctcagccagcaccaagggcccttccgtgttcccc |
| | | | 82562 | ctggccccttgcagcaggagcacctccgaatccacagctgccctgggctgtctggtgaaggactact |
| | | | heavy | ttcccgagcccgtgaccgtgagctggaacagcggcgctctgacatccggcgtccacacctttcctgc |
| | | | chain | cgtcctgcagtcctccggcctctactccctgtcctccgtggtgaccgtgcctagctcctccctcggc |
| | | | | accaagacctacacctgtaacgtggaccacaaaccctccaacaccaaggtggacaaacgggtcgaga |
| | | | | gcaagtacggccctccctgccctccttgtcctgcccccgagttcgaaggcggacccagcgtgttcct |
| | | | | gttccctcctaagcccaaggacaccctcatgatcagccggacacccgaggtgacctgcgtggtggtg |
| | | | | gatgtgagccaggaggaccctgaggtccagttcaactggtatgtggatggcgtggaggtgcacaacg |
| | | | | ccaagacaaagccccgggaagagcagttcaactccacctacagggtggtcagcgtgctgaccgtgct |
| | | | | gcatcaggactggctgaacggcaaggagtacaagtgcaaggtcagcaataagggactgcccagcagc |
| | | | | atcgagaagaccatctccaaggctaaaggccagccccgggaacctcaggtgtacaccctgcctccca |
| | | | | gccaggaggagatgaccaagaaccaggtgagcctgacctgcctggtgaagggattctacccttccga |
| | | | | catcgccgtggagtgggagtccaacggccagcccgagaacaattataagaccacccctcccgtcctc |
| | | | | gacagcgacggatccttctttctgtactccaggctgaccgtggataagtccaggtggcaggaaggca |
| | | | | acgtgttcagctgctccgtgatgcacgaggccctgcacaatcactacacccagaagtccctgagcct |
| | | | | gtccctgggaaag |
| 148 | CL- | Light | Amino | DIQMTQSPSSLSASVGDRVTITCRASQGIRNDLGWYQQKPGKAPKRLIYGASSLQSGVPSRFSGS |
| | 82562 | chain | acid | GSGTEFTLTISSLQPEDFATYYCLQHNSYPRTFGQGTKVEIK |
| | | vari- | se- | |
| | | able | quence | |
| | | region | of V_{L} | |
| | | | of CL- | |
| | | | 82562 | |
| 149 | CL- | Light | Nu- | gacatccagatgacccagtctccatcctccctgtctgcatctgtaggagacagagtcaccatcactt |
| | 82562 | chain | cleic | gccgggcaagtcagggcattagaaatgatttaggctggtatcagcagaagccagggaaagcccctaa |
| | | vari- | acid | gcgcctgatctatggtgcatccagtttgcaaagtggagtcccatcaaggttcagcggcagtggatct |
| | | able | se- | gggacagaattcactctcacaatcagcagcctgcagcctgaagattttgcaacttattactgtctac |
| | | region | quence | agcataatagttaccctcggacgttcggccaagggaccaaggtggaaatcaaac |
| | | | of V_{L} | |
| | | | of CL- | |
| | | | 82562 | |
| 150 | CL- | Full | Amino | DIQMTQSPSSLSASVGDRVTITCRASQGIRNDLGWYQQKPGKAPKRLIYGASSLQSGVPSRFSGS |
| | 82562 | light | acid | GSGTEFTLTISSLQPEDFATYYCLQHNSYPRTFGQGTKVEIKRTVAAPSVFIFPPSDEQLKSGTASV |
| | | chain | se- | VCLLNNFYPREAKVQWKVDNALQSGNSQESVTEQDSKDSTYSLSSTLTLSKADYEKHKVYACEVT |
| | | se- | quence | HQGLSSPVTKSFNRGEC |
| | | quence | of CL- | |
| | | | 82562 | |
| | | | light | |
| | | | chain | |
| 151 | CL- | Full | Nu- | gacatccagatgacccagtctccatcctccctgtctgcatctgtaggagacagagtcaccatcactt |
| | 82562 | light | cleic | gccgggcaagtcagggcattagaaatgatttaggctggtatcagcagaagccagggaaagcccctaa |
| | | chain | acid | gcgcctgatctatggtgcatccagtttgcaaagtggagtcccatcaaggttcagcggcagtggatct |
| | | se- | se- | gggacagaattcactctcacaatcagcagcctgcagcctgaagattttgcaacttattactgtctac |
| | | quence | quence | agcataatagttaccctcggacgttcggccaagggaccaaggtggaaatcaaacgtacggtggccgc |
| | | | of CL- | tccctccgtgttcatcttcccaccttccgacgagcagctgaagtccggcaccgcttctgtcgtgtgc |
| | | | 82562 | ctgctgaacaacttctacccccgcgaggccaaggtgcagtggaaggtggacaacgccctgcagtccg |
| | | | light | gcaactcccaggaatccgtgaccgagcaggactccaaggacagcacctactccctgtcctccaccct |
| | | | chain | gaccctgtccaaggccgactacgagaagcacaaggtgtacgcctgcgaagtgacccaccagggcctg |
| | | | | tctagccccgtgaccaagtctttcaaccggggcgagtgt |
| 152 | CL- | Heavy | Amino | QVQLVESGGGVVQPGRSLRLSCAASGFTFYSYGMHWVRQAPGKGLEWVAVIWYGGTNKYYADS |
| | 82556 | chain | acid | VKGRFTISRDNSKNTLYLQMNSLTAEDTAVYYCARGQLERRRGGLNIWGQGTMVTVSS |
| | | vari- | se- | |
| | | able | quence | |
| | | region | of V_{H} | |
| | | | of CL- | |
| | | | 82556 | |
| 153 | CL- | Heavy | Nu- | caggtgcagctggtggagtctgggggaggcgtggtccagcctgggaggtccctgagactctcctgtg |
| | 82556 | chain | cleic | cagcgtctggattcaccttctatagctatggcatgcactgggtccgccaggctccaggcaaggggct |
| | | vari- | acid | ggagtgggtggcagttatatggtatggcggaactaataaatactatgcagactccgtgaagggccga |
| | | able | se- | ttcaccatctccagagacaattccaagaatacgctgtatctgcaaatgaacagcctgacagccgaag |
| | | region | quence | acacggctgtgtattactgtgcgagagggcaattggaacgccgacggggcggtttgaatatctgggg |
| | | | of V_{H} | ccaagggacaatggtcaccgtctcttcag |
| | | | of CL- | |
| | | | 82556 | |
| 154 | CL- | Full | Amino | QVQLVESGGGVVQPGRSLRLSCAASGFTFYSYGMHWVRQAPGKGLEWVAVIWYGGTNKYYADS |
| | 82556 | heavy | acid | VKGRFTISRDNSKNTLYLQMNSLTAEDTAVYYCARGQLERRRGGLNIWGQGTMVTVSSASTKGP |
| | | chain | se- | SVFPLAPCSRSTSESTAALGCLVKDYFPEPVTVSWNSGALTSGVHTFPAVLQSSGLYSLSSVVTVP |
| | | se- | quence | SSSLGTKTYTCNVDHKPSNTKVDKRVESKYGPPCPPCPAPEFEGGPSVFLFPPKPKDTLMISRTPE |
| | | quence | of CL- | VTCVVVDVSQEDPEVQFNWYVDGVEVHNAKTKPREEQFNSTYRVVSVLTVLHQDWLNGKEYKC |
| | | | 82556 | KVSNKGLPSSIEKTISKAKGQPREPQVYTLPPSQEEMTKNQVSLTCLVKGFYPSDIAVEWESNGQP |
| | | | heavy | ENNYKTTPPVLDSDGSFFLYSRLTVDKSRWQEGNVFSCSVMHEALHNHYTQKSLSLSLGK |
| | | | chain | |
| 155 | CL- | Full | Nu- | caggtgcagctggtggagtctgggggaggcgtggtccagcctgggaggtccctgagactctcctgtg |
| | 82556 | heavy | cleic | cagcgtctggattcaccttctatagctatggcatgcactgggtccgccaggctccaggcaaggggct |
| | | chain | acid | ggagtgggtggcagttatatggtatggcggaactaataaatactatgcagactccgtgaagggccga |
| | | se- | se- | ttcaccatctccagagacaattccaagaatacgctgtatctgcaaatgaacagcctgacagccgaag |
| | | quence | quence | acacggctgtgtattactgtgcgagagggcaattggaacgccgacggggcggtttgaatatctgggg |
| | | | of CL- | ccaagggacaatggtcaccgtctcttcagccagcaccaagggcccttccgtgttccccctggcccct |
| | | | 82556 | tgcagcaggagcacctccgaatccacagctgccctgggctgtctggtgaaggactactttcccgagc |
| | | | heavy | ccgtgaccgtgagctggaacagcggcgctctgacatccggcgtccacacctttcctgccgtcctgca |
| | | | chain | gtcctccggcctctactccctgtcctccgtggtgaccgtgcctagctcctccctcggcaccaagacc |
| | | | | tacacctgtaacgtggaccacaaaccctccaacaccaaggtggacaaacgggtcgagagcaagtacg |
| | | | | gccctccctgccctccttgtcctgcccccgagttcgaaggcggacccagcgtgttcctgttccctcc |
| | | | | taagcccaaggacaccctcatgatcagccggacacccgaggtgacctgcgtggtggtggatgtgagc |
| | | | | caggaggaccctgaggtccagttcaactggtatgtggatggcgtggaggtgcacaacgccaagacaa |
| | | | | agccccgggaagagcagttcaactccacctacagggtggtcagcgtgctgaccgtgctgcatcagga |
| | | | | ctggctgaacggcaaggagtacaagtgcaaggtcagcaataagggactgcccagcagcatcgagaag |
| | | | | accatctccaaggctaaaggccagccccgggaacctcaggtgtacaccctgcctcccagccaggagg |
| | | | | agatgaccaagaaccaggtgagcctgacctgcctggtgaagggattctacccttccgacatcgccgt |
| | | | | ggagtgggagtccaacggccagcccgagaacaattataagaccacccctcccgtcctcgacagcgac |
| | | | | ggatccttctttctgtactccaggctgaccgtggataagtccaggtggcaggaaggcaacgtgttca |
| | | | | gctgctccgtgatgcacgaggccctgcacaatcactacacccagaagtccctgagcctgtccctggg |
| | | | | aaag |
| 156 | CL- | Light | Amino | DIQMTQSPSSLSASVGDRVTITCRASQGVRDDLGWYQQKPGKAPKRLIYAASNLQSGVPSRFSG |
| | 82556 | chain | acid | SGSGTEFTLTIGSLQPEDFATYYCLQHNSYPFTFGPGTKVDIK |
| | | vari- | se- | |
| | | able | quence | |
| | | region | of V_{L} | |
| | | | of CL- | |
| | | | 82556 | |
| 157 | CL- | Light | Nu- | gacatccagatgacccagtctccatcctccctgtctgcatctgtaggagacagagtcaccatcactt |
| | 82556 | chain | cleic | gccgggcaagtcagggcgttagagatgatttaggttggtatcagcagaaaccagggaaagcccctaa |
| | | vari- | acid | gcgcctgatctatgctgcatccaatttgcaaagtggggtcccatcaaggttcagcggcagtggatct |
| | | able | se- | gggacagaattcactctcacaatcggcagcctgcagcctgaagattttgcaacttattactgtctac |
| | | region | quence | agcataatagttatccattcactttcggccctgggaccaaagtggatatcaaac |
| | | | of V_{L} | |
| | | | of CL- | |
| | | | 82556 | |
| 158 | CL- | Full | Amino | DIQMTQSPSSLSASVGDRVTITCRASQGVRDDLGWYQQKPGKAPKRLIYAASNLQSGVPSRFSG |
| | 82556 | light | acid | SGSGTEFTLTIGSLQPEDFATYYCLQHNSYPFTFGPGTKVDIKRTVAAPSVFIFPPSDEQLKSGTAS |
| | | chain | se- | WVCLLNNFYPREAKVQWKVDNALQSGNSQESVTEQDSKDSTYSLSSTLTLSKADYEKHKVYACEV |
| | | se- | quence | THQGLSSPVTKSFNRGEC |
| | | quence | of CL- | |
| | | | 82556 | |
| | | | light | |
| | | | chain | |
| 159 | CL- | Full | Nu- | gacatccagatgacccagtctccatcctccctgtctgcatctgtaggagacagagtcaccatcactt |
| | 82556 | light | cleic | gccgggcaagtcagggcgttagagatgatttaggttggtatcagcagaaaccagggaaagcccctaa |
| | | chain | acid | gcgcctgatctatgctgcatccaatttgcaaagtggggtcccatcaaggttcagcggcagtggatct |
| | | se- | se- | gggacagaattcactctcacaatcggcagcctgcagcctgaagattttgcaacttattactgtctac |
| | | quence | quence | agcataatagttatccattcactttcggccctgggaccaaagtggatatcaaacgtacggtggccgc |
| | | | of CL- | tccctccgtgttcatcttcccaccttccgacgagcagctgaagtccggcaccgcttctgtcgtgtgc |
| | | | 82556 | ctgctgaacaacttctacccccgcgaggccaaggtgcagtggaaggtggacaacgccctgcagtccg |
| | | | light | gcaactcccaggaatccgtgaccgagcaggactccaaggacagcacctactccctgtcctccaccct |
| | | | chain | gaccctgtccaaggccgactacgagaagcacaaggtgtacgcctgcgaagtgacccaccagggcctg |
| | | | | tctagccccgtgaccaagtctttcaaccggggcgagtgt |
| 160 | CL- | Heavy | Amino | QVQLVQSGAEVKKPGASVKVSCKASGYTFTSYDINWVRQATGQGLEWMGWMNPNSGNTGYAQ |
| | 82551 | chain | acid | KFQGRVTMTRNTSISTAYMELSSLRSEDTAVYYCARGRGYYGSGSHYRGYYYYGLDVWGQGTTV |
| | | vari- | se- | TVSS |
| | | able | quence | |
| | | region | of V_{H} | |
| | | | of CL- | |
| | | | 82551 | |
| 161 | CL- | Heavy | Nu- | caggtgcagctggtgcagtctggggctgaggtgaagaagcctggggcctcagtgaaggtctcctgca |
| | 82551 | chain | cleic | aggcttctggatacaccttcaccagttatgatatcaactgggtgcgacaggccactggacaagggct |
| | | vari- | acid | tgagtggatgggatggatgaaccctaacagtggtaacacaggctatgcacagaagttccagggcaga |
| | | able | se- | gtcaccatgaccaggaacacctccataagcacagcctacatggagctgagcagcctgagatctgagg |
| | | region | quence | acacggccgtgtattactgtgcgagaggccgggggtactatggttcggggagccattataggggcta |
| | | | of V_{H} | ctactactacggtttggacgtctggggccaagggaccacggtcaccgtctcctcag |
| | | | of CL- | |
| | | | 82551 | |
| 162 | CL- | Full | Amino | QVQLVQSGAEVKKPGASVKVSCKASGYTFTSYDINWVRQATGQGLEWMGWMNPNSGNTGYAQ |
| | 82551 | heavy | acid | KFQGRVTMTRNTSISTAYMELSSLRSEDTAVYYCARGRGYYGSGSHYRGYYYYGLDVWGQGTTV |
| | | chain | se- | TVSSASTKGPSVFPLAPCSRSTSESTAALGCLVKDYFPEPVTVSWNSGALTSGVHTFPAVLQSSGL |
| | | se- | quence | YSLSSVVTVPSSSLGTKTYTCNVDHKPSNTKVDKRVESKYGPPCPPCPAPEFEGGPSVFLFPPKPK |
| | | quence | of CL- | DTLMISRTPEVTCVVVDVSQEDPEVQFNWYVDGVEVHNAKTKPREEQFNSTYRVVSVLTVLHQD |
| | | | 82551 | WLNGKEYKCKVSNKGLPSSIEKTISKAKGQPREPQVYTLPPSQEEMTKNQVSLTCLVKGFYPSDIA |
| | | | heavy | VEWESNGQPENNYKTTPPVLDSDGSFFLYSRLTVDKSRWQEGNVFSCSVMHEALHNHYTQKSLS |
| | | | chain | LSLGK |
| 163 | CL- | Full | Nu- | caggtgcagctggtgcagtctggggctgaggtgaagaagcctggggcctcagtgaaggtctcctgca |
| | 82551 | heavy | cleic | aggcttctggatacaccttcaccagttatgatatcaactgggtgcgacaggccactggacaagggct |
| | | chain | acid | tgagtggatgggatggatgaaccctaacagtggtaacacaggctatgcacagaagttccagggcaga |
| | | se- | se- | gtcaccatgaccaggaacacctccataagcacagcctacatggagctgagcagcctgagatctgagg |
| | | quence | quence | acacggccgtgtattactgtgcgagaggccgggggtactatggttcggggagccattataggggcta |
| | | | of CL- | ctactactacggtttggacgtctggggccaagggaccacggtcaccgtctcctcagccagcaccaag |
| | | | 82551 | ggcccttccgtgttccccctggccccttgcagcaggagcacctccgaatccacagctgccctgggct |
| | | | heavy | gtctggtgaaggactactttcccgagcccgtgaccgtgagctggaacagcggcgctctgacatccgg |
| | | | chain | cgtccacacctttcctgccgtcctgcagtcctccggcctctactccctgtcctccgtggtgaccgtg |
| | | | | cctagctcctccctcggcaccaagacctacacctgtaacgtggaccacaaaccctccaacaccaagg |
| | | | | ggacaaacgggtcgagagcaagtacggccctccctgccctccttgtcctgcccccgagttcgaaggc |
| | | | | ggacccagcgtgttcctgttccctcctaagcccaaggacaccctcatgatcagccggacacccgagg |
| | | | | tgacctgcgtggtggtggatgtgagccaggaggaccctgaggtccagttcaactggtatgtggatgg |
| | | | | cgtggaggtgcacaacgccaagacaaagccccgggaagagcagttcaactccacctacagggtggtc |
| | | | | agcgtgctgaccgtgctgcatcaggactggctgaacggcaaggagtacaagtgcaaggtcagcaata |
| | | | | agggactgcccagcagcatcgagaagaccatctccaaggctaaaggccagccccgggaacctcaggt |
| | | | | gtacaccctgcctcccagccaggaggagatgaccaagaaccaggtgagcctgacctgcctggtgaag |
| | | | | ggattctacccttccgacatcgccgtggagtgggagtccaacggccagcccgagaacaattataaga |
| | | | | ccacccctcccgtcctcgacagcgacggatccttctttctgtactccaggctgaccgtggataagtc |
| | | | | caggtggcaggaaggcaacgtgttcagctgctccgtgatgcacgaggccctgcacaatcactacacc |
| | | | | cagaagtccctgagcctgtccctgggaaag |
| 164 | CL- | Light | Amino | DIQMTQSPSTLSASVGDRVTITCRASQSISSWLAWYQQKPGKAPKVLIYKASSLKSGVPSRFSGS |
| | 82551 | chain | acid | GSGTEFTLTVSSLQPDDFATYYCQQYNSYSWTFGQGTKVEIK |
| | | vari- | se- | |
| | | able | quence | |
| | | region | of V_{L} | |
| | | | of CL- | |
| | | | 82551 | |
| 165 | CL- | Light | Nu- | gacatccagatgacccagtctccttccaccctgtctgcatctgtaggagacagagtcaccatcactt |
| | 82551 | chain | cleic | gccgggccagtcagagtattagtagctggttggcctggtatcagcagaaaccagggaaagcccctaa |
| | | vari- | acid | ggtcctgatctataaggcgtctagtttaaaaagtggggtcccatcaaggttcagcggcagtggatct |
| | | able | se- | gggacagaattcactctcaccgtcagcagcctgcagcctgatgattttgcaacttattactgccaac |
| | | region | quence | agtataatagttattcgtggacgttcggccaagggaccaaggtggaaatcaaac |
| | | | of V_{L} | |
| | | | of CL- | |
| | | | 82551 | |
| 166 | CL- | Full | Amino | DIQMTQSPSTLSASVGDRVTITCRASQSISSWLAWYQQKPGKAPKVLIYKASSLKSGVPSRFSGS |
| | 82551 | light | acid | GSGTEFTLTVSSLQPDDFATYYCQQYNSYSWTFGQGTKVEIKRTVAAPSVFIFPPSDEQLKSGTA |
| | | chain | se- | SVVCLLNNFYPREAKVQWKVDNALQSGNSQESVTEQDSKDSTYSLSSTLTLSKADYEKHKVYACE |
| | | se- | quence | VTHQGLSSPVTKSFNRGEC |
| | | quence | of CL- | |
| | | | 82551 | |
| | | | light | |
| | | | chain | |
| 167 | CL- | Full | Nu- | gacatccagatgacccagtctccttccaccctgtctgcatctgtaggagacagagtcaccatcactt |
| | 82551 | light | cleic | gccgggccagtcagagtattagtagctggttggcctggtatcagcagaaaccagggaaagcccctaa |
| | | chain | acid | ggtcctgatctataaggcgtctagtttaaaaagtggggtcccatcaaggttcagcggcagtggatct |
| | | se- | se- | gggacagaattcactctcaccgtcagcagcctgcagcctgatgattttgcaacttattactgccaac |
| | | quence | quence | agtataatagttattcgtggacgttcggccaagggaccaaggtggaaatcaaacgtacggtggccgc |
| | | | of CL- | tccctccgtgttcatcttcccaccttccgacgagcagctgaagtccggcaccgcttctgtcgtgtgc |
| | | | 82551 | ctgctgaacaacttctacccccgcgaggccaaggtgcagtggaaggtggacaacgccctgcagtccg |
| | | | light | gcaactcccaggaatccgtgaccgagcaggactccaaggacagcacctactccctgtcctccaccct |
| | | | chain | gaccctgtccaaggccgactacgagaagcacaaggtgtacgcctgcgaagtgacccaccagggcctg |
| | | | | tctagccccgtgaccaagtctttcaaccggggcgagtgt |
| 168 | CL- | Heavy | Amino | QVQLQESGPGLVKPSQTLSLTCTVSGGSISSGRYYWSWIRQLPGKGLEWIGYISYSGNPFYSPSL |
| | 82541 | chain | acid | KSRVTISVDTSENQFSLKLSSVTAADTAVYYCARDRRIPMLRGVKGYYYGMDVWGQGTTVTVSS |
| | | vari- | se- | |
| | | able | quence | |
| | | region | of V_{H} | |
| | | | of CL- | |
| | | | 82541 | |
| 169 | CL- | Heavy | Nu- | caggtgcagctgcaggagtcgggcccaggactggtgaagccttcacagaccctgtccctcacctgca |
| | 82541 | chain | cleic | ctgtctctggtggctccatcagcagtggtagatactactggagctggatccgccagctcccagggaa |
| | | vari- | acid | gggcctggagtggattgggtacatctcatacagtgggaaccccttctacagcccgtccctcaagagt |
| | | able | se- | cgagttaccatatcagtagacacgtctgagaaccaattctccctgaagctgagctctgtgactgccg |
| | | region | quence | cggacacggccgtgtattactgtgcgagagatcgacgtattcctatgcttcggggagttaaaggcta |
| | | | of V_{H} | ctactacggtatggacgtctggggccaagggaccacggtcaccgtctcctcag |
| | | | of CL- | |
| | | | 82541 | |
| 170 | CL- | Full | Amino | QVQLQESGPGLVKPSQTLSLTCTVSGGSISSGRYYWSWIRQLPGKGLEWIGYISYSGNPFYSPSL |
| | 82541 | heavy | acid | KSRVTISVDTSENQFSLKLSSVTAADTAVYYCARDRRIPMLRGVKGYYYGMDVWGQGTTVTVSS |
| | | chain | se- | ASTKGPSVFPLAPCSRSTSESTAALGCLVKDYFPEPVTVSWNSGALTSGVHTFPAVLQSSGLYSLS |
| | | se- | quence | SVVTVPSSSLGTKTYTCNVDHKPSNTKVDKRVESKYGPPCPPCPAPEFEGGPSVFLFPPKPKDTLM |
| | | quence | of CL- | ISRTPEVTCVVVDVSQEDPEVQFNWYVDGVEVHNAKTKPREEQFNSTYRVVSVLTVLHQDWLNG |
| | | | 82541 | KEYKCKVSNKGLPSSIEKTISKAKGQPREPQVYTLPPSQEEMTKNQVSLTCLVKGFYPSDIAVEWE |
| | | | heavy | SNGQPENNYKTTPPVLDSDGSFFLYSRLTVDKSRWQEGNVFSCSVMHEALHNHYTQKSLSLSLG |
| | | | chain | K |
| 171 | CL- | Full | Nu- | caggtgcagctgcaggagtcgggcccaggactggtgaagccttcacagaccctgtccctcacctgca |
| | 82541 | heavy | cleic | ctgtctctggtggctccatcagcagtggtagatactactggagctggatccgccagctcccagggaa |
| | | chain | acid | gggcctggagtggattgggtacatctcatacagtgggaaccccttctacagcccgtccctcaagagt |
| | | se- | se- | cgagttaccatatcagtagacacgtctgagaaccaattctccctgaagctgagctctgtgactgccg |
| | | quence | quence | cggacacggccgtgtattactgtgcgagagatcgacgtattcctatgcttcggggagttaaaggcta |
| | | | of CL- | ctactacggtatggacgtctggggccaagggaccacggtcaccgtctcctcagccagcaccaagggc |
| | | | 82541 | ccttccgtgttccccctggccccttgcagcaggagcacctccgaatccacagctgccctgggctgtc |
| | | | heavy | tggtgaaggactactttcccgagcccgtgaccgtgagctggaacagcggcgctctgacatccggcgt |
| | | | chain | ccacacctttcctgccgtcctgcagtcctccggcctctactccctgtcctccgtggtgaccgtgcct |
| | | | | agctcctccctcggcaccaagacctacacctgtaacgtggaccacaaaccctccaacaccaaggtgg |
| | | | | acaaacgggtcgagagcaagtacggccctccctgccctccttgtcctgcccccgagttcgaaggcgg |
| | | | | acccagcgtgttcctgttccctcctaagcccaaggacaccctcatgatcagccggacacccgaggtg |
| | | | | acctgcgtggtggtggatgtgagccaggaggaccctgaggtccagttcaactggtatgtggatggcg |
| | | | | tggaggtgcacaacgccaagacaaagccccgggaagagcagttcaactccacctacagggtggtcag |
| | | | | cgtgctgaccgtgctgcatcaggactggctgaacggcaaggagtacaagtgcaaggtcagcaataag |
| | | | | ggactgcccagcagcatcgagaagaccatctccaaggctaaaggccagccccgggaacctcaggtgt |
| | | | | acaccctgcctcccagccaggaggagatgaccaagaaccaggtgagcctgacctgcctggtgaaggg |
| | | | | attctacccttccgacatcgccgtggagtgggagtccaacggccagcccgagaacaattataagacc |
| | | | | acccctcccgtcctcgacagcgacggatccttctttctgtactccaggctgaccgtggataagtcca |
| | | | | ggtggcaggaaggcaacgtgttcagctgctccgtgatgcacgaggccctgcacaatcactacaccca |
| | | | | gaagtccctgagcctgtccctgggaaag |
| 172 | CL- | Light | Amino | DIVMTQSPLSLPVIPGEPASISCRSSQSLLHSNGYNYLDWYLQKPGQSPHLLIYLGSNRASGVPDR |
| | 82541 | chain | acid | FSGSGSGTDFTLKISRVEAEDVGVYYCMQALQTPWTFGQGTKVEIK |
| | | vari- | se- | |
| | | able | quence | |
| | | region | of V_{L} | |
| | | | of CL- | |
| | | | 82541 | |
| 173 | CL- | Light | Nu- | gatattgtgatgactcagtctccactctccctgcccgtcatccctggagagccggcctccatctcct |
| | 82541 | chain | cleic | gcaggtctagtcagagcctcctgcatagtaatggatacaactatttggattggtacctgcagaagcc |
| | | vari- | acid | agggcagtctccacacctcctgatctatttgggttctaatcgggcctccggggtccc |
| | | able | se- | tgacaggttcagtggcagtggatcaggcacagattttacactgaaaatcagcagagtg |
| | | region | quence | gaggctgaggatgttggggtttattactgcatgcaagctctacaaactccgtggacgttcggcca |
| | | | of V_{L} | agggaccaaggtggaaatcaaac |
| | | | of CL- | |
| | | | 82541 | |
| 174 | CL- | Full | Amino | DIVMTQSPLSLPVIPGEPASISCRSSQSLLHSNGYNYLDWYLQKPGQSPHLLIYLGSNRASGVPDR |
| | 82541 | light | acid | FSGSGSGTDFTLKISRVEAEDVGVYYCMQALQTPWTFGQGTKVEIKRTVAAPSVFIFPPSDEQLK |
| | | chain | se- | SGTASVVCLLNNFYPREAKVQWKVDNALQSGNSQESVTEQDSKDSTYSLSSTLTLSKADYEKHK |
| | | se- | quence | VYACEVTHQGLSSPVTKSFNRGEC |
| | | quence | of CL- | |
| | | | 82541 | |
| | | | light | |
| | | | chain | |
| 175 | CL- | Full | Nu- | gatattgtgatgactcagtctccactctccctgcccgtcatccctggagagccggcctccatctc |
| | 82541 | light | cleic | ctgcaggtctagtcagagcctcctgcatagtaatggatacaactatttggattggtacctgcag |
| | | chain | acid | aagccagggcagtctccacacctcctgatctatttgggttctaatcgggcctccggggtccct |
| | | se- | se- | gacaggttcagtggcagtggatcaggcacagattttacactgaaaatcagcagagtggaggctg |
| | | quence | quence | aggatgttggggtttattactgcatgcaagctctacaaactccgtggacgttcggccaagggac |
| | | | of CL- | caaggtggaaatcaaacgtacggtggccgctccctccgtgttcatcttcccaccttccgacg |
| | | | 82541 | agcagctgaagtccggcaccgcttctgtcgtgtgcctgctgaacaacttctacccccgcgagg |
| | | | light | ccaaggtgcagtggaaggtggacaacgccctgcagtccggcaactcccaggaatccgtgac |
| | | | chain | cgagcaggactccaaggacagcacctactccctgtcctccaccctgaccctgtccaaggcc |
| | | | | gactacgagaagcacaaggtgtacgcctgcgaagtgacccaccagggcctgtctagcc |
| | | | | ccgtgaccaagtctttcaaccggggcgagtgt |
| 176 | CL- | Heavy | Amino | EVQLVESGGGLVQPGGSLRLSCAASGFTFSNYAMSWVRQAPRKGLEWVSVISGSGGRTNYAGSV |
| | 82523 | chain | acid | KGRFSITRDNSKNTLYLQMNSLRAEDTAVYYCVKGQLERRYYYYYGMDVWGQGITVTVSS |
| | | vari- | se- | |
| | | able | quence | |
| | | region | of V_{H} | |
| | | | of CL- | |
| | | | 82523 | |
| 177 | CL- | Heavy | Nu- | gaggtgcagctggtggagtctgggggaggcttggtacagcctggggggtccctgagactctc |
| | 82523 | chain | cleic | ctgtgcagcctctggattcacctttagcaactatgccatgagctgggtccgccaggctcc |
| | | vari- | acid | aaggaaggggctggagtgggtctcagttattagtggtagtggtggtagaacaaactacgc |
| | | able | se- | aggctccgtgaagggccggttctccatcaccagagacaattccaagaacacgctgtatctg |
| | | region | quence | caaatgaacagcctgagagccgaggacacggccgtatattactgtgtgaaaggccaattgg |
| | | | of V_{H} | aacgacggtactactactattacggtatggacgtctggggccaagggattacggtcaccgt |
| | | | of CL- | ctcctcag |
| | | | 82523 | |
| 178 | CL- | Full | Amino | EVQLVESGGGLVQPGGSLRLSCAASGFTFSNYAMSWVRQAPRKGLEWVSVISGSGGRTNYAGSV |
| | 82523 | heavy | acid | KGRFSITRDNSKNTLYLQMNSLRAEDTAVYYCVKGQLERRYYYYYGMDVWGQGITVTVSSASTK |
| | | chain | se- | GPSVFPLAPCSRSTSESTAALGCLVKDYFPEPVTVSWNSGALTSGVHTFPAVLQSSGLYSLSSVVT |
| | | se- | quence | VPSSSLGTKTYTCNVDHKPSNTKVDKRVESKYGPPCPPCPAPEFEGGPSVFLFPPKPKDTLMISRT |
| | | quence | of CL- | PEVTCVVVDVSQEDPEVQFNWYVDGVEVHNAKTKPREEQFNSTYRVVSVLTVLHQDWLNGKEY |
| | | | 82523 | KCKVSNKGLPSSIEKTISKAKGQPREPQVYTLPPSQEEMTKNQVSLTCLVKGFYPSDIAVEWESNG |
| | | | heavy | QPENNYKTTPPVLDSDGSFFLYSRLTVDKSRWQEGNVFSCSVMHEALHNHYTQKSLSLSLGK |
| | | | chain | |
| 179 | CL- | Full | Nu- | gaggtgcagctggtggagtctgggggaggcttggtacagcctggggggtccctgagactctcctgt |
| | 82523 | heavy | cleic | gcagcctctggattcacctttagcaactatgccatgagctgggtccgccaggctccaaggaaggg |
| | | chain | acid | gctggagtgggtctcagttattagtggtagtggtggtagaacaaactacgcaggctccgtgaagg |
| | | se- | se- | gccggttctccatcaccagagacaattccaagaacacgctgtatctgcaaatgaacagcctgagag |
| | | quence | quence | ccgaggacacggccgtatattactgtgtgaaaggccaattggaacgacggtactactactattacg |
| | | | of CL- | gtatggacgtctggggccaagggattacggtcaccgtctcctcagccagcaccaagggcccttccg |
| | | | 82523 | tgttccccctggccccttgcagcaggagcacctccgaatccacagctgccctgggctgtctggtga |
| | | | heavy | aggactactttcccgagcccgtgaccgtgagctggaacagcggcgctctgacatccggcgtccaca |
| | | | chain | cctttcctgccgtcctgcagtcctccggcctctactccctgtcctccgtggtgaccgtgcctagctc |
| | | | | ctccctcggcaccaagacctacacctgtaacgtggaccacaaaccctccaacaccaaggtggacaaa |
| | | | | cgggtcgagagcaagtacggccctccctgccctccttgtcctgcccccgagttcgaaggcggaccca |
| | | | | gcgtgttcctgttccctcctaagcccaaggacaccctcatgatcagccggacacccgaggtgacct |
| | | | | gcgtggtggtggatgtgagccaggaggaccctgaggtccagttcaactggtatgtggatggcgtgg |
| | | | | aggtgcacaacgccaagacaaagccccgggaagagcagttcaactccacctacagggtggtcagcg |
| | | | | tgctgaccgtgctgcatcaggactggctgaacggcaaggagtacaagtgcaaggtcagcaataa |
| | | | | gggactgcccagcagcatcgagaagaccatctccaaggctaaaggccagccccgggaacctc |
| | | | | aggtgtacaccctgcctcccagccaggaggagatgaccaagaaccaggtgagcctgacctgc |
| | | | | ctggtgaagggattctacccttccgacatcgccgtggagtgggagtccaacggccagcccg |
| | | | | agaacaattataagaccacccctcccgtcctcgacagcgacggatccttctttctgtactc |
| | | | | caggctgaccgtggataagtccaggtggcaggaaggcaacgtgttcagctgctccgtga |
| | | | | tgcacgaggccctgcacaatcactacacccagaagtccctgagcctgtccctgggaaag |
| 180 | CL- | Light | Amino | DIQMTQSPSSLSASVGDRVTITCRASQGIRNDLGWYQQKPGKAPKRLIYAASSLQSGVPSRFSGS |
| | 82523 | chain | acid | GSGTEFTLTISSLQPEDFATYYCLQHNSYPLTFGGGTKVEIK |
| | | vari- | se- | |
| | | able | quence | |
| | | region | of V_{L} | |
| | | | of CL- | |
| | | | 82523 | |
| 181 | CL- | Light | Nu- | gacatccagatgacccagtctccatcctccctgtctgcatctgtaggagacagagtcaccat |
| | 82523 | chain | cleic | cacttgccgggcaagtcagggcattagaaatgatttaggctggtatcagcagaaa |
| | | vari- | acid | ccagggaaagcccctaagcgcctgatctatgctgcatccagtttgcaaagtggggtcc |
| | | able | se- | catcaaggttcagcggcagtggatctgggacagaattcactctcacaatcagcagcct |
| | | region | quence | gcagcctgaagattttgcaacttattactgtctacagcataatagttacccg |
| | | | of V_{L} | ctcactttcggcggagggaccaaggtggagatcaaac |
| | | | of CL- | |
| | | | 82523 | |
| 182 | CL- | Full | Amino | DIQMTQSPSSLSASVGDRVTITCRASQGIRNDLGWYQQKPGKAPKRLIYAASSLQSGVPSRFSGS |
| | 82523 | light | acid | GSGTEFTLTISSLQPEDFATYYCLQHNSYPLTFGGGTKVEIKRTVAAPSVFIFPPSDEQLKSGTASV |
| | | chain | se- | VCLLNNFYPREAKVQWKVDNALQSGNSQESVTEQDSKDSTYSLSSTLTLSKADYEKHKVYACEVT |
| | | se- | quence | HQGLSSPVTKSFNRGEC |
| | | quence | of CL- | |
| | | | 82523 | |
| | | | light | |
| | | | chain | |
| 183 | CL- | Full | Nu- | gacatccagatgacccagtctccatcctccctgtctgcatctgtaggagacagagtcaccatc |
| | 82523 | light | cleic | acttgccgggcaagtcagggcattagaaatgatttaggctggtatcagcagaaaccaggg |
| | | chain | acid | aaagcccctaagcgcctgatctatgctgcatccagtttgcaaagtggggtcccatcaagg |
| | | se- | se- | ttcagcggcagtggatctgggacagaattcactctcacaatcagcagcctgcagcctgaag |
| | | quence | quence | attttgcaacttattactgtctacagcataatagttacccgctcactttcggcggagggac |
| | | | of CL- | caaggtggagatcaaacgtacggtggccgctccctccgtgttcatcttcccaccttccgac |
| | | | 82523 | gagcagctgaagtccggcaccgcttctgtcgtgtgcctgctgaacaacttctacccccg |
| | | | light | cgaggccaaggtgcagtggaaggtggacaacgccctgcagtccggcaactcccaggaat |
| | | | chain | ccgtgaccgagcaggactccaaggacagcacctactccctgtcctccaccctgaccct |
| | | | | gtccaaggccgactacgagaagcacaaggtgtacgcctgcgaagtgacccac |
| | | | | cagggcctgtctagccccgtgaccaagtctttcaaccggggcgagtgt |
| 184 | CL- | Heavy | Amino | EVQLVESGGGLVKPGGSLRLSCAASGFTFSSYSMNWVRQAPGKGLEWVSSISSSRRYIYYADSVK |
| | 82517 | chain | acid | GRFTISRDNAKNSLYLQMNSLRAEDTAVYYCARAYGQVAPYYYYGMDVWGQGTTVTVSS |
| | | vari- | se- | |
| | | able | quence | |
| | | region | of V_{H} | |
| | | | of CL- | |
| | | | 82517 | |
| 185 | CL- | Heavy | Nu- | gaggtgcagctggtggagtctgggggaggcctggtcaagcctggggggtccctgagactctcct |
| | 82517 | chain | cleic | gtgcagcctctggattcaccttcagtagctatagcatgaactgggtccgccaggctccaggga |
| | | vari- | acid | aggggctggagtgggtctcatccattagtagtagtagacgttacatatactacgcagactc |
| | | able | se- | agtgaagggccgattcaccatctccagagacaacgccaagaactcactgtatctgcaaatg |
| | | region | quence | aacagcctgagagccgaggacacagctgtgtattactgtgcgagagcctatgggcaggtcg |
| | | | of V_{H} | ccccctactactactacggtatggacgtctggggccaagggaccacggtcaccgtctcctcag |
| | | | of CL- | |
| | | | 82517 | |
| 186 | CL- | Full | Amino | EVQLVESGGGLVKPGGSLRLSCAASGFTFSSYSMNWVRQAPGKGLEWVSSISSSRRYIYYADSVK |
| | 82517 | heavy | acid | GRFTISRDNAKNSLYLQMNSLRAEDTAVYYCARAYGQVAPYYYYGMDVWGQGTTVTVSSASTK |
| | | chain | se- | GPSVFPLAPCSRSTSESTAALGCLVKDYFPEPVTVSWNSGALTSGVHTFPAVLQSSGLYSLSSVVT |
| | | se- | quence | VPSSSLGTKTYTCNVDHKPSNTKVDKRVESKYGPPCPPCPAPEFEGGPSVFLFPPKPKDTLMISRT |
| | | quence | of CL- | PEVTCVVVDVSQEDPEVQFNWYVDGVEVHNAKTKPREEQFNSTYRVVSVLTVLHQDWLNGKEY |
| | | | 82517 | KCKVSNKGLPSSIEKTISKAKGQPREPQVYTLPPSQEEMTKNQVSLTCLVKGFYPSDIAVEWESNG |
| | | | heavy | QPENNYKTTPPVLDSDGSFFLYSRLTVDKSRWQEGNVFSCSVMHEALHNHYTQKSLSLSLGK |
| | | | chain | |
| 187 | CL- | Full | Nu- | gaggtgcagctggtggagtctgggggaggcctggtcaagcctggggggtccctgagactctcct |
| | 82517 | heavy | cleic | gtgcagcctctggattcaccttcagtagctatagcatgaactgggtccgccaggctccaggga |
| | | chain | acid | aggggctggagtgggtctcatccattagtagtagtagacgttacatatactacgcagactc |
| | | se- | se- | agtgaagggccgattcaccatctccagagacaacgccaagaactcactgtatctgcaaatg |
| | | quence | quence | aacagcctgagagccgaggacacagctgtgtattactgtgcgagagcctatgggcaggtcg |
| | | | of CL- | ccccctactactactacggtatggacgtctggggccaagggaccacggtcaccgtctcctca |
| | | | 82517 | gccagcaccaagggcccttccgtgttccccctggccccttgcagcaggagcacctccg |
| | | | heavy | aatccacagctgccctgggctgtctggtgaaggactactttcccgagcccgtgaccgt |
| | | | chain | gagctggaacagcggcgctctgacatccggcgtccacacctttcctgccgtcctgcag |
| | | | | tcctccggcctctactccctgtcctccgtggtgaccgtgcctagctcctccctcgg |
| | | | | caccaagacctacacctgtaacgtggaccacaaaccctccaacaccaaggtggacaa |
| | | | | acgggtcgagagcaagtacggccctccctgccctccttgtcctgcccccgagttcga |
| | | | | aggcggacccagcgtgttcctgttccctcctaagcccaaggacaccctcatgatc |
| | | | | agccggacacccgaggtgacctgcgtggtggtggatgtgagccaggaggaccctgag |
| | | | | gtccagttcaactggtatgtggatggcgtggaggtgcacaacgccaagacaaagccccgggaaga |
| | | | | gcagttcaactccacctacagggtggtcagcgtgctgaccgtgctgcatcaggactggctgaacg |
| | | | | gcaaggagtacaagtgcaaggtcagcaataagggactgcccagcagcatcgagaagaccatc |
| | | | | tccaaggctaaaggccagccccgggaacctcaggtgtacaccctgcctcccagccaggagga |
| | | | | gatgaccaagaaccaggtgagcctgacctgcctggtgaagggattctacccttccgacat |
| | | | | cgccgtggagtgggagtccaacggccagcccgagaacaattataagaccacccctcccgtcc |
| | | | | tcgacagcgacggatccttctttctgtactccaggctgaccgtggataagtccaggtggcag |
| | | | | gaaggcaacgtgttcagctgctccgtgatgcacgaggccctgcacaatcactacac |
| | | | | ccagaagtccctgagcctgtccctgggaaag |
| 188 | CL- | Light | Amino | AIQLTQSPSSLSASVGDRVTITCRASQGISSALAWYQQKPGKAPKLLIYDASSLESGVPSRFSGSG |
| | 82517 | chain | acid | SGTDFTLTISSLQPEDFATYYCQQFNSYPLTFGGGTKVEIK |
| | | vari- | se- | |
| | | able | quence | |
| | | region | of V_{L} | |
| | | | of CL- | |
| | | | 82517 | |
| 189 | CL- | Light | Nu- | gccatccagttgacccagtctccatcctccctgtctgcatctgtaggagacagagtcaccatc |
| | 82517 | chain | cleic | acttgccgggcaagtcagggcattagcagtgctttagcctggtatcagcagaaaccagggaaagc |
| | | vari- | acid | tcctaaactcctgatctatgatgcctccagtttggaaagtggggtcccatcaaggttcagcgg |
| | | able | se- | cagtggatctgggacagatttcactctcaccatcagcagcctgcagcctgaagattttgcaac |
| | | region | quence | ttattactgtcaacagtttaatagttacccgctcactttcggcggagggaccaaggtggagat |
| | | | of V_{L} | caaac |
| | | | of CL- | |
| | | | 82517 | |
| 190 | CL- | Full | Amino | AIQLTQSPSSLSASVGDRVTITCRASQGISSALAWYQQKPGKAPKLLIYDASSLESGVPSRFSGSG |
| | 82517 | light | acid | SGTDFTLTISSLQPEDFATYYCQQFNSYPLTFGGGTKVEIKRTVAAPSVFIFPPSDEQLKSGTASVV |
| | | chain | se- | CLLNNFYPREAKVQWKVDNALQSGNSQESVTEQDSKDSTYSLSSTLTLSKADYEKHKVYACEVT |
| | | se- | quence | HQGLSSPVTKSFNRGEC |
| | | quence | of CL- | |
| | | | 82517 | |
| | | | light | |
| | | | chain | |
| 191 | CL- | Full | Nu- | gccatccagttgacccagtctccatcctccctgtctgcatctgtaggagacagagtcaccatc |
| | 82517 | light | cleic | acttgccgggcaagtcagggcattagcagtgctttagcctggtatcagcagaaaccagggaaa |
| | | chain | acid | gctcctaaactcctgatctatgatgcctccagtttggaaagtggggtcccatcaaggttcagcg |
| | | se- | se- | gcagtggatctgggacagatttcactctcaccatcagcagcctgcagcctgaagattttgcaa |
| | | quence | quence | cttattactgtcaacagtttaatagttacccgctcactttcggcggagggaccaaggtggaga |
| | | | of CL- | tcaaacgtacggtggccgctccctccgtgttcatcttcccaccttccgacgagcagctgaagt |
| | | | 82517 | ccggcaccgcttctgtcgtgtgcctgctgaacaacttctacccccgcgaggccaaggtgcag |
| | | | light | tggaaggtggacaacgccctgcagtccggcaactcccaggaatccgtgaccgagcaggactcc |
| | | | chain | aaggacagcacctactccctgtcctccaccctgaccctgtccaaggccgactacgagaagcac |
| | | | | aaggtgtacgcctgcgaagtgacccaccagggcctgtctagccccgtgaccaagtct |
| | | | | ttcaaccggggcgagtgt |
| 192 | CL- | Heavy | Amino | QVQLVESGGGVVQPGRSLRLSCEASGFTLSSYGMHWVRQAPGKGLEWVAVIWNDGSNKYSADS |
| | 82495 | chain | acid | VKGRFTISRDNSKNTLYLQMNRLRAEDTAVYYCARGGGYYGSGSHYRGYYYYGMDVWGQGTTV |
| | | vari- | se- | TVSS |
| | | able | quence | |
| | | region | of V_{H} | |
| | | | of CL- | |
| | | | 82495 | |
| 193 | CL- | Heavy | Nu- | caggtgcagctggtggagtctgggggaggcgtggtccagcctgggaggtccctgagactctcc |
| | 82495 | chain | cleic | tgtgaagcgtctggattcaccctcagtagctatggcatgcactgggtccgccaggctccagg |
| | | vari- | acid | caaggggctggagtgggtggcagttatatggaatgatggaagtaataaatattctgcagact |
| | | able | se- | ccgtgaagggccgattcaccatctccagagacaattccaagaacaccctgtatctgcaaatg |
| | | region | quence | aacagactgagagccgaggacacggctgtgtattattgtgcgagagggggtggttactatgg |
| | | | of V_{H} | ttcggggagtcattatagaggatactattactacggtatggacgtctggggcc |
| | | | of CL- | aagggaccacggtcaccgtctcctcag |
| | | | 82495 | |
| 194 | CL- | Full | Amino | QVQLVESGGGVVQPGRSLRLSCEASGFTLSSYGMHWVRQAPGKGLEWVAVIWNDGSNKYSADS |
| | 82495 | heavy | acid | VKGRFTISRDNSKNTLYLQMNRLRAEDTAVYYCARGGGYYGSGSHYRGYYYYGMDVWGQGTTV |
| | | chain | se- | TVSSASTKGPSVFPLAPCSRSTSESTAALGCLVKDYFPEPVTVSWNSGALTSGVHTFPAVLQSSGL |
| | | se- | quence | YSLSSVVTVPSSSLGTKTYTCNVDHKPSNTKVDKRVESKYGPPCPPCPAPEFEGGPSVFLFPPKPK |
| | | quence | of CL- | DTLMISRTPEVTCVVVDVSQEDPEVQFNWYVDGVEVHNAKTKPREEQFNSTYRVVSVLTVLHQD |
| | | | 82495 | WLNGKEYKCKVSNKGLPSSIEKTISKAKGQPREPQVYTLPPSQEEMTKNQVSLTCLVKGFYPSDIA |
| | | | heavy | VEWESNGQPENNYKTTPPVLDSDGSFFLYSRLTVDKSRWQEGNVFSCSVMHEALHNHYTQKSLS |
| | | | chain | LSLGK |
| 195 | CL- | Full | Nu- | caggtgcagctggtggagtctgggggaggcgtggtccagcctgggaggtccctgagactctcc |
| | 82495 | heavy | cleic | tgtgaagcgtctggattcaccctcagtagctatggcatgcactgggtccgccaggctccag |
| | | chain | acid | gcaaggggctggagtgggtggcagttatatggaatgatggaagtaataaatattctgcaga |
| | | se- | se- | ctccgtgaagggccgattcaccatctccagagacaattccaagaacaccctgtatctgcaa |
| | | quence | quence | atgaacagactgagagccgaggacacggctgtgtattattgtgcgagagggggtggttact |
| | | | of CL- | atggttcggggagtcattatagaggatactattactacggtatggacgtctggggccaagg |
| | | | 82495 | gaccacggtcaccgtctcctcagccagcaccaagggcccttccgtgttccccctggcccct |
| | | | heavy | tgcagcaggagcacctccgaatccacagctgccctgggctgtctggtgaaggactactttc |
| | | | chain | ccgagcccgtgaccgtgagctggaacagcggcgctctgacatccggcgtccacacctttcc |
| | | | | tgccgtcctgcagtcctccggcctctactccctgtcctccgtggtgaccgtgcctagctcc |
| | | | | tccctcggcaccaagacctacacctgtaacgtggaccacaaaccctccaacaccaaggtgg |
| | | | | acaaacgggtcgagagcaagtacggccctccctgccctccttgtcctgcccccgagttcga |
| | | | | aggcggacccagcgtgttcctgttccctcctaagcccaaggacaccctcatgatcagccgg |
| | | | | acacccgaggtgacctgcgtggtggtggatgtgagccaggaggaccctgaggtccagttca |
| | | | | actggtatgtggatggcgtggaggtgcacaacgccaagacaaagccccgggaagagcagttca |
| | | | | actccacctacagggtggtcagcgtgctgaccgtgctgcatcaggactggctgaacggcaa |
| | | | | ggagtacaagtgcaaggtcagcaataagggactgcccagcagcatcgagaagaccatctcc |
| | | | | aaggctaaaggccagccccgggaacctcaggtgtacaccctgcctcccagccaggaggaga |
| | | | | tgaccaagaaccaggtgagcctgacctgcctggtgaagggattctacccttccgacatcg |
| | | | | ccgtggagtgggagtccaacggccagcccgagaacaattataagaccacccctcccgtc |
| | | | | ctcgacagcgacggatccttctttctgtactccaggctgaccgtggataagtccaggtgg |
| | | | | caggaaggcaacgtgttcagctgctccgtgatgcacgaggccctgcacaatcacta |
| | | | | cacccagaagtccctgagcctgtccctgggaaag |
| 196 | CL- | Light | Amino | DIQMTQSPSSLSASVGDRVTITCRASQGIRNDLGWYQQKPGKAPKRLIYAASSLQSGVPSRF |
| | 82495 | chain | acid | SGSGSGTEFTLTISSLQPEDFATYYCLQHNSYPWTFGQGTKVEIK |
| | | vari- | se- | |
| | | able | quence | |
| | | region | of V_{L} | |
| | | | of CL- | |
| | | | 82495 | |
| 197 | CL- | Light | Nu- | gacatccagatgacccagtctccatcctccctgtctgcatctgtaggagacagagtca |
| | 82495 | chain | cleic | ccatcacttgccgggcaagtcagggcattagaaatgatttaggctggtatcagcagaa |
| | | vari- | acid | accagggaaagcccctaagcgcctgatctatgctgcatccagtttgcaaagtggg |
| | | able | se- | gtcccatcaaggttcagcggcagtggatctgggacagaattcactctcacaatcag |
| | | region | quence | cagcctgcagcctgaagattttgcaacttattactgtctacagcataatagtt |
| | | | of V_{L} | acccgtggacgttcggccaagggaccaaggtggaaatcaaac |
| | | | of CL- | |
| | | | 82495 | |
| 198 | CL- | Full | Amino | DIQMTQSPSSLSASVGDRVTITCRASQGIRNDLGWYQQKPGKAPKRLIYAASSLQSGVPSRFSGS |
| | 82495 | light | acid | GSGTEFTLTISSLQPEDFATYYCLQHNSYPWTFGQGTKVEIKRTVAAPSVFIFPPSDEQLKSGTAS |
| | | chain | se- | VVCLLNNFYPREAKVQWKVDNALQSGNSQESVTEQDSKDSTYSLSSTLTLSKADYEKHKVYACEV |
| | | se- | quence | THQGLSSPVTKSFNRGEC |
| | | quence | of CL- | |
| | | | 82495 | |
| | | | light | |
| | | | chain | |
| 199 | CL- | Full | Nu- | gacatccagatgacccagtctccatcctccctgtctgcatctgtaggagacagagtcaccatca |
| | 82495 | light | cleic | cttgccgggcaagtcagggcattagaaatgatttaggctggtatcagcagaaaccagggaaag |
| | | chain | acid | cccctaagcgcctgatctatgctgcatccagtttgcaaagtggggtcccatcaaggttcagcg |
| | | se- | se- | gcagtggatctgggacagaattcactctcacaatcagcagcctgcagcctgaagattttgcaa |
| | | quence | quence | cttattactgtctacagcataatagttacccgtggacgttcggccaagggaccaaggtggaaa |
| | | | of CL- | tcaaacgtacggtggccgctccctccgtgttcatcttcccaccttccgacgagcagctgaag |
| | | | 82495 | tccggcaccgcttctgtcgtgtgcctgctgaacaacttctacccccgcgaggccaaggtgcag |
| | | | light | tggaaggtggacaacgccctgcagtccggcaactcccaggaatccgtgaccgagcaggact |
| | | | chain | ccaaggacagcacctactccctgtcctccaccctgaccctgtccaaggccgactacgagaa |
| | | | | gcacaaggtgtacgcctgcgaagtgacccaccagggcctgtctagccccgtgaccaa |
| | | | | gtctttcaaccggggcgagtgt |
| 200 | CL- | Heavy | Amino | QVQLVESGGGVVQPGRSLRLSCAASGFTFSSYGMHWVRQAPGKGLEWVTVILYGGSNKYYADS |
| | 82473 | chain | acid | VKGRFTISRDNSKNTLYLQMNSLRAEDTAVYYCARGMLTYYYSSGFDYWGQGTLVTVSS |
| | | vari- | se- | |
| | | able | quence | |
| | | region | of V_{H} | |
| | | | of CL- | |
| | | | 82473 | |
| 201 | CL- | Heavy | Nu- | caggtgcagctggtggagtctgggggaggcgtggtccagcctgggaggtccctgagactctc |
| | 82473 | chain | cleic | ctgtgcagcgtctggattcaccttcagtagctatggcatgcactgggtccgccaggctccag |
| | | vari- | acid | gcaaggggctggagtgggtaacagttatattgtatggtggaagtaataaatactatgcaga |
| | | able | se- | ctccgtgaagggccgtttcaccatctccagagacaattccaagaacacgctgtatctgc |
| | | region | quence | aaatgaacagcctcagagccgaggacacggctgtgtattactgtgcgagagggatgttaac |
| | | | of V_{H} | gtattactatagttcggggtttgactactggggccagggaaccctggtcaccgtctcctcag |
| | | | of CL- | |
| | | | 82473 | |
| 202 | CL- | Full | Amino | QVQLVESGGGVVQPGRSLRLSCAASGFTFSSYGMHWVRQAPGKGLEWVTVILYGGSNKYYADS |
| | 82473 | heavy | acid | VKGRFTISRDNSKNTLYLQMNSLRAEDTAVYYCARGMLTYYYSSGFDYWGQGTLVTVSSASTKG |
| | | chain | se- | PSVFPLAPCSRSTSESTAALGCLVKDYFPEPVTVSWNSGALTSGVHTFPAVLQSSGLYSLSSVVTV |
| | | se- | quence | PSSSLGTKTYTCNVDHKPSNTKVDKRVESKYGPPCPPCPAPEFEGGPSVFLFPPKPKDTLMISRTP |
| | | quence | of CL- | EVTCVVVDVSQEDPEVQFNWYVDGVEVHNAKTKPREEQFNSTYRVVSVLTVLHQDWLNGKEYK |
| | | | 82473 | CKVSNKGLPSSIEKTISKAKGQPREPQVYTLPPSQEEMTKNQVSLTCLVKGFYPSDIAVEWESNG |
| | | | heavy | QPENNYKTTPPVLDSDGSFFLYSRLTVDKSRWQEGNVFSCSVMHEALHNHYTQKSLSLSLGK |
| | | | chain | |
| 203 | CL- | Full | Nu- | caggtgcagctggtggagtctgggggaggcgtggtccagcctgggaggtccctgagactctcc |
| | 82473 | heavy | cleic | tgtgcagcgtctggattcaccttcagtagctatggcatgcactgggtccgccaggctcca |
| | | chain | acid | ggcaaggggctggagtgggtaacagttatattgtatggtggaagtaataaatactatgcaga |
| | | se- | se- | ctccgtgaagggccgtttcaccatctccagagacaattccaagaacacgctgtatctgc |
| | | quence | quence | aaatgaacagcctcagagccgaggacacggctgtgtattactgtgcgagagggatgttaacg |
| | | | of CL- | tattactatagttcggggtttgactactggggccagggaaccctggtcaccgtctcctcagc |
| | | | 82473 | cagcaccaagggcccttccgtgttccccctggccccttgcagcaggagcacctccgaatcca |
| | | | heavy | cagctgccctgggctgtctggtgaaggactactttcccgagcccgtgaccgtgagctgg |
| | | | chain | aacagcggcgctctgacatccggcgtccacacctttcctgccgtcctgcagtcctccggcct |
| | | | | ctactccctgtcctccgtggtgaccgtgcctagctcctccctcggcaccaagacctacacct |
| | | | | gtaacgtggaccacaaaccctccaacaccaaggtggacaaacgggtcgagagcaagtacggc |
| | | | | cctccctgccctccttgtcctgcccccgagttcgaaggcggacccagcgtgttcctgttccc |
| | | | | tcctaagcccaaggacaccctcatgatcagccggacacccgaggtgacctgcgtggtggtgg |
| | | | | atgtgagccaggaggaccctgaggtccagttcaactggtatgtggatggcgtggaggtgcac |
| | | | | aacgccaagacaaagccccgggaagagcagttcaactccacctacagggtggtcagcgtgct |
| | | | | gaccgtgctgcatcaggactggctgaacggcaaggagtacaagtgcaaggtcagcaataag |
| | | | | ggactgcccagcagcatcgagaagaccatctccaaggctaaaggccagccccgggaacctca |
| | | | | ggtgtacaccctgcctcccagccaggaggagatgaccaagaaccaggtgagcctgacctgcc |
| | | | | tggtgaagggattctacccttccgacatcgccgtggagtgggagtccaacggccagcccgag |
| | | | | aacaattataagaccacccctcccgtcctcgacagcgacggatccttctttctgtactccag |
| | | | | gctgaccgtggataagtccaggtggcaggaaggcaacgtgttcagctgctccgtgatgcacg |
| | | | | aggccctgcacaatcactacacccagaagtccctgagcctgtccctgggaaag |
| 204 | CL- | Light | Amino | DVVMTQSPLSLPVTLGQPASISCRSSQSLVYSDGNTYLNWFQQRPGQSPRRLIYKVSNRDSG |
| | 82473 | chain | acid | VPDRFSGSGSGTDFTLKISRVEAEDVGVYYCMQGTHWPFTFGPGTKVDIK |
| | | vari- | se- | |
| | | able | quence | |
| | | region | of V_{L} | |
| | | | of CL- | |
| | | | 82473 | |
| 205 | CL- | Light | Nu- | gatgttgtgatgactcagtctccactctccctgcccgtcacccttggacagccggcctccat |
| | 82473 | chain | cleic | ctcctgcaggtctagtcaaagcctcgtatacagtgatggaaacacctacttgaattggtttc |
| | | vari- | acid | agcagaggccaggccaatctccaaggcgcctaatttataaggtttctaaccgggactctggg |
| | | able | se- | gtcccagacagattcagcggcagtgggtcaggcactgatttcacactgaaaatcagcagggt |
| | | region | quence | ggaggctgaggatgttggggtttattactgcatgcaaggtacacactggccattcactttcg |
| | | | of V_{L} | gccctgggaccaaagtggatatcaaac |
| | | | of CL- | |
| | | | 82473 | |
| 206 | CL- | Full | Amino | DVVMTQSPLSLPVTLGQPASISCRSSQSLVYSDGNTYLNWFQQRPGQSPRRLIYKVSNRDSGVP |
| | 82473 | light | acid | DRFSGSGSGTDFTLKISRVEAEDVGVYYCMQGTHWPFTFGPGTKVDIKRTVAAPSVFIFPPSDEQ |
| | | chain | se- | LKSGTASVVCLLNNFYPREAKVQWKVDNALQSGNSQESVTEQDSKDSTYSLSSTLTLSKADYEKH |
| | | se- | quence | KVYACEVTHQGLSSPVTKSFNRGEC |
| | | quence | of CL- | |
| | | | 82473 | |
| | | | light | |
| | | | chain | |
| 207 | CL- | Full | Nu- | gatgttgtgatgactcagtctccactctccctgcccgtcacccttggacagccggcctccat |
| | 82473 | light | cleic | ctcctgcaggtctagtcaaagcctcgtatacagtgatggaaacacctacttgaattggtttc |
| | | chain | acid | agcagaggccaggccaatctccaaggcgcctaatttataaggtttctaaccgggactctggg |
| | | se- | se- | gtcccagacagattcagcggcagtgggtcaggcactgatttcacactgaaaatcagcagggt |
| | | quence | quence | ggaggctgaggatgttggggtttattactgcatgcaaggtacacactggccattcactttcg |
| | | | of CL- | gccctgggaccaaagtggatatcaaacgtacggtggccgctccctccgtgttcatcttccca |
| | | | 82473 | ccttccgacgagcagctgaagtccggcaccgcttctgtcgtgtgcctgctgaacaacttcta |
| | | | light | cccccgcgaggccaaggtgcagtggaaggtggacaacgccctgcagtccggcaactcccagg |
| | | | chain | aatccgtgaccgagcaggactccaaggacagcacctactccctgtcctccaccctgaccctg |
| | | | | tccaaggccgactacgagaagcacaaggtgtacgcctgcgaagtgacccaccagggcctgt |
| | | | | ctagccccgtgaccaagtctttcaaccggggcgagtgt |
| 208 | CL- | Heavy | Amino | QVQLVESGGGVVQPGRSLRLSCAASGFTFNNYGMHWVRQAPGKGLEWVAVIWNDGSNKYYGD |
| | 82455 | chain | acid | SVKGRFTISRDNSKNTLYLQMNSLRAEDTAVYYCARGGGYYGSGKHYRGYYYNGMGVWGQGTP |
| | | vari- | se- | VTVSS |
| | | able | quence | |
| | | region | of V_{H} | |
| | | | of CL- | |
| | | | 82455 | |
| 209 | CL- | Heavy | Nu- | caggtgcagttggtggagtctgggggaggcgtggtccagcctgggaggtccctgagactctc |
| | 82455 | chain | cleic | ctgtgcagcgtctggattcaccttcaataactatggcatgcactgggtccgccaggctcca |
| | | vari- | acid | ggcaaggggctggagtgggtggcagttatatggaatgatggaagtaataaatactatggag |
| | | able | se- | actccgtgaagggccgattcaccatctccagagacaattccaagaacacgctgtatctgca |
| | | region | quence | aatgaacagcctgagagccgaggacacggctgtgtattactgtgcgagagggggtggttac |
| | | | of V_{H} | tatggttcggggaaacattatagaggctattactataacggtatgggcgtctggggccaag |
| | | | of CL- | ggaccccggtcaccgtctcctcag |
| | | | 82455 | |
| 210 | CL- | Full | Amino | QVQLVESGGGVVQPGRSLRLSCAASGFTFNNYGMHWVRQAPGKGLEWVAVIWNDGSNKYYGD |
| | 82455 | heavy | acid | SVKGRFTISRDNSKNTLYLQMNSLRAEDTAVYYCARGGGYYGSGKHYRGYYYNGMGVWGQGTP |
| | | chain | se- | VTVSSASTKGPSVFPLAPCSRSTSESTAALGCLVKDYFPEPVTVSWNSGALTSGVHTFPAVLQ |
| | | se- | quence | SSGLYSLSSVVTVPSSSLGTKTYTCNVDHKPSNTKVDKRVESKYGPPCPPCPAPEFEGGPSVF |
| | | quence | of CL- | LFPPKPKDTLMISRTPEVTCVVVDVSQEDPEVQFNWYVDGVEVHNAKTKPREEQFNSTYRVVS |
| | | | 82455 | VLTVLHQDWLNGKEYKCKVSNKGLPSSIEKTISKAKGQPREPQVYTLPPSQEEMTKNQVSLTC |
| | | | heavy | LVKGFYPSDIAVEWESNGQPENNYKTTPPVLDSDGSFFLYSRLTVDKSRWQEGNVFSCSVMHE |
| | | | chain | ALHNHYTQKSLSLSLGK |
| 211 | CL- | Full | Nu- | caggtgcagttggtggagtctgggggaggcgtggtccagcctgggaggtccctgagactctcc |
| | 82455 | heavy | cleic | tgtgcagcgtctggattcaccttcaataactatggcatgcactgggtccgccaggctccaggc |
| | | chain | acid | aaggggctggagtgggtggcagttatatggaatgatggaagtaataaatactatggagactcc |
| | | se- | se- | gtgaagggccgattcaccatctccagagacaattccaagaacacgctgtatctgcaaatgaac |
| | | quence | quence | agcctgagagccgaggacacggctgtgtattactgtgcgagagggggtggttactatggttcg |
| | | | of CL- | gggaaacattatagaggctattactataacggtatgggcgtctggggccaagggaccccggtc |
| | | | 82455 | accgtctcctcagccagcaccaagggcccttccgtgttccccctggccccttgcagcaggagc |
| | | | heavy | acctccgaatccacagctgccctgggctgtctggtgaaggactactttcccgagcccgtgacc |
| | | | chain | gtgagctggaacagcggcgctctgacatccggcgtccacacctttcctgccgtcctgcagtcc |
| | | | | tccggcctctactccctgtcctccgtggtgaccgtgcctagctcctccctcggcaccaagacc |
| | | | | tacacctgtaacgtggaccacaaaccctccaacaccaaggtggacaaacgggtcgagagcaag |
| | | | | tacggccctccctgccctccttgtcctgcccccgagttcgaaggcggacccagcgtgttcctg |
| | | | | ttccctcctaagcccaaggacaccctcatgatcagccggacacccgaggtgacctgcgtggtg |
| | | | | gtggatgtgagccaggaggaccctgaggtccagttcaactggtatgtggatggcgtggaggtg |
| | | | | cacaacgccaagacaaagccccgggaagagcagttcaactccacctacagggtggtcagcgtg |
| | | | | ctgaccgtgctgcatcaggactggctgaacggcaaggagtacaagtgcaaggtcagcaataag |
| | | | | ggactgcccagcagcatcgagaagaccatctccaaggctaaaggccagccccgggaacctcag |
| | | | | gtgtacaccctgcctcccagccaggaggagatgaccaagaaccaggtgagcctgacctgcctg |
| | | | | gtgaagggattctacccttccgacatcgccgtggagtgggagtccaacggccagcccgagaac |
| | | | | aattataagaccacccctcccgtcctcgacagcgacggatccttctttctgtactccaggctg |
| | | | | accgtggataagtccaggtggcaggaaggcaacgtgttcagctgctccgtgatgcacgaggc |
| | | | | cctgcacaatcactacacccagaagtccctgagcctgtccctgggaaag |
| 212 | CL- | Light | Amino | EIVMTQSPATLSVSPGERATLSCRASQSVSSNLAWYQQKPGQAPRLLIYGASTRATGIPARFS |
| | 82455 | chain | acid | GSGSGTEVTLTISSLQSEDFTIYYCQQYNNWPWTFGQGTKVDIK |
| | | vari- | se- | |
| | | able | quence | |
| | | region | of V_{L} | |
| | | | of CL- | |
| | | | 82455 | |
| 213 | CL- | Light | Nu- | gaaatagtgatgacgcagtctccagccaccctgtctgtgtctccaggggaaagagccaccctc |
| | 82455 | chain | cleic | tcctgcagggccagtcagagtgttagcagcaacttagcctggtaccagcagaaacctggccag |
| | | vari- | acid | gctcccaggctcctcatctatggtgcatccaccagggccactggtatcccagccaggttcagt |
| | | able | se- | ggcagtgggtctgggacagaggtcactctcaccatcagcagcctgcagtctgaagattttaca |
| | | region | quence | atttattactgtcagcagtataataactggccgtggacgttcggccaagggaccaaggtggat |
| | | | of V_{L} | atcaaac |
| | | | of CL- | |
| | | | 82455 | |
| 214 | CL- | Full | Amino | EIVMTQSPATLSVSPGERATLSCRASQSVSSNLAWYQQKPGQAPRLLIYGASTRATGIPARFSGS |
| | 82455 | light | acid | GSGTEVTLTISSLQSEDFTIYYCQQYNNWPWTFGQGTKVDIKRTVAAPSVFIFPPSDEQLKSGTA |
| | | chain | se- | SVVCLLNNFYPREAKVQWKVDNALQSGNSQESVTEQDSKDSTYSLSSTLTLSKADYEKHKVYACE |
| | | se- | quence | VTHQGLSSPVTKSFNRGEC |
| | | quence | of CL- | |
| | | | CL- | |
| | | | 82455 | |
| | | | light | |
| | | | chain | |
| 215 | CL- | Full | Nu- | gaaatagtgatgacgcagtctccagccaccctgtctgtgtctccaggggaaagagccaccctc |
| | 82455 | light | cleic | tcctgcagggccagtcagagtgttagcagcaacttagcctggtaccagcagaaacctggccag |
| | | chain | acid | gctcccaggctcctcatctatggtgcatccaccagggccactggtatcccagccaggttcagt |
| | | se- | se- | ggcagtgggtctgggacagaggtcactctcaccatcagcagcctgcagtctgaagattttaca |
| | | quence | quence | atttattactgtcagcagtataataactggccgtggacgttcggccaagggaccaaggtggat |
| | | | of CL- | atcaaacgtacggtggccgctccctccgtgttcatcttcccaccttccgacgagcagctgaag |
| | | | CL- | tccggcaccgcttctgtcgtgtgcctgctgaacaacttctacccccgcgaggccaaggtgcag |
| | | | 82455 | tggaaggtggacaacgccctgcagtccggcaactcccaggaatccgtgaccgagcaggactc |
| | | | light | caaggacagcacctactccctgtcctccaccctgaccctgtccaaggccgactacgagaag |
| | | | chain | cacaaggtgtacgcctgcgaagtgacccaccagggcctgtctagccccgtgaccaagtcttt |
| | | | | caaccggggcgagtgt |
| 216 | CL- | Heavy | Amino | QVQLQQWGAGLLKPSETLSLTCAVYGGSFSGYYWSWIRQPPGKGLEWIGKINHSGSTNYKPSLK |
| | 83158 | chain | acid | SRVNISVDTSKNQFSLKLTSVTAADTAVYYCAREASMLRGVKGYRFYLYAMDVWGQGTSVTVSS |
| | | vari- | se- | |
| | | able | quence | |
| | | region | of V_{H} | |
| | | | of CL- | |
| | | | 83158 | |
| 217 | CL- | Heavy | Nu- | caggtgcagctacaacagtggggcgcaggactgttgaagccttcggagaccctgtccctcac |
| | 83158 | chain | cleic | ctgcgctgtctatggtgggtccttcagtggttactactggagttggatccgccagcccccag |
| | | vari- | acid | ggaaggggctggagtggattgggaaaatcaatcatagtggaagcaccaactacaaaccgtcc |
| | | able | se- | ctcaagagtcgagtcaacatatcagtagacacgtccaagaaccagttctccctgaagctgac |
| | | region | quence | ctctgtgaccgccgcggacacggctgtgtattactgtgcgagagaggcctctatgcttcggg |
| | | | of V_{H} | gagttaaggggtatcgcttctacttatacgcaatggacgtctggggccaaggg |
| | | | of CL- | acctcggtcaccgtctcctcag |
| | | | 83158 | |
| 218 | CL- | Full | Amino | QVQLQQWGAGLLKPSETLSLTCAVYGGSFSGYYWSWIRQPPGKGLEWIGKINHSGSTNYKPSLK |
| | 83158 | heavy | acid | SRVNISVDTSKNQFSLKLTSVTAADTAVYYCAREASMLRGVKGYRFYLYAMDVWGQGTSVTVSS |
| | | chain | se- | ASTKGPSVFPLAPCSRSTSESTAALGCLVKDYFPEPVTVSWNSGALTSGVHTFPAVLQSSGLYSLS |
| | | se- | quence | SVVTVPSSSLGTKTYTCNVDHKPSNTKVDKRVESKYGPPCPPCPAPEFEGGPSVFLFPPKPKDTLM |
| | | quence | of CL- | ISRTPEVTCVVVDVSQEDPEVQFNWYVDGVEVHNAKTKPREEQFNSTYRVVSVLTVLHQDWLNG |
| | | | CL- | KEYKCKVSNKGLPSSIEKTISKAKGQPREPQVYTLPPSQEEMTKNQVSLTCLVKGFYPSDIAVEWE |
| | | | 83158 | SNGQPENNYKTTPPVLDSDGSFFLYSRLTVDKSRWQEGNVFSCSVMHEALHNHYTQKSLSLSLG |
| | | | heavy | KDSVKGRFTISRDNSKNTLYLQMNSLRAEDTAVYYCARGGGYYGSGKYYRGYYYNGMDVWGQG |
| | | | chain | TPVTVSSASTKGPSVFPLAPCSRSTSESTAALGCLVKDYFPEPVTVSWNSGALTSGVHTFPAVLQS |
| | | | | SGLYSLSSVVTVPSSSLGTKTYTCNVDHKPSNTKVDKRVESKYGPPCPPCPAPEFEGGPSVFLFPP |
| | | | | KPKDTLMISRTPEVTCVVVDVSQEDPEVQFNWYVDGVEVHNAKTKPREEQFNSTYRVVSVLTVL |
| | | | | HQDWLNGKEYKCKVSNKGLPSSIEKTISKAKGQPREPQVYTLPPSQEEMTKNQVSLTCLVKGFYP |
| | | | | SDIAVEWESNGQPENNYKTTPPVLDSDGSFFLYSRLTVDKSRWQEGNVFSCSVMHEALHNHYTQ |
| | | | | KSLSLSLGK |
| 219 | CL- | Full | Nu- | caggtgcagctacaacagtggggcgcaggactgttgaagccttcggagaccctgtccctcac |
| | 83158 | heavy | cleic | ctgcgctgtctatggtgggtccttcagtggttactactggagttggatccgccagcccccag |
| | | chain | acid | ggaaggggctggagtggattgggaaaatcaatcatagtggaagcaccaactacaaacc |
| | | se- | se- | gtccctcaagagtcgagtcaacatatcagtagacacgtccaagaaccagttctccctgaagc |
| | | quence | quence | tgacctctgtgaccgccgcggacacggctgtgtattactgtgcgagagaggcctctatgctt |
| | | | of CL- | cggggagttaaggggtatcgcttctacttatacgcaatggacgtctggggccaagggacctc |
| | | | CL- | ggtcaccgtctcctcagccagcaccaagggcccttccgtgttccccctggccccttgcagca |
| | | | 83158 | ggagcacctccgaatccacagctgccctgggctgtctggtgaaggactactttcccgagcc |
| | | | heavy | cgtgaccgtgagctggaacagcggcgctctgacatccggcgtccacacctttcctgccgtcc |
| | | | chain | tgcagtcctccggcctctactccctgtcctccgtggtgaccgtgcctagctcctccctcggc |
| | | | | accaagacctacacctgtaacgtggaccacaaaccctccaacaccaaggtggacaaacgggt |
| | | | | cgagagcaagtacggccctccctgccctccttgtcctgcccccgagttcgaaggcggaccca |
| | | | | gcgtgttcctgttccctcctaagcccaaggacaccctcatgatcagccggacacccgaggtg |
| | | | | acctgcgtggtggtggatgtgagccaggaggaccctgaggtccagttcaactggtatgtgg |
| | | | | atggcgtggaggtgcacaacgccaagacaaagccccgggaagagcagttcaactccacctac |
| | | | | agggtggtcagcgtgctgaccgtgctgcatcaggactggctgaacggcaaggagtacaagt |
| | | | | gcaaggtcagcaataagggactgcccagcagcatcgagaagaccatctccaaggctaaagg |
| | | | | ccagccccgggaacctcaggtgtacaccctgcctcccagccaggaggagatgaccaagaac |
| | | | | caggtgagcctgacctgcctggtgaagggattctacccttccgacatcgccgtggagtggg |
| | | | | agtccaacggccagcccgagaacaattataagaccacccctcccgtcctcgacagcgacgg |
| | | | | atccttctttctgtactccaggctgaccgtggataagtccaggtggcaggaaggcaacgtg |
| | | | | ttcagctgctccgtgatgcacgaggccctgcacaatcactacacccagaagt |
| | | | | ccctgagcctgtccctgggaaag |
| 220 | CL- | Light | Amino | SSELTQDPAVSVALGQTVRITCQGDSLRSYYASWYQQKPGQTPLLVIYGKNNRPSGIPDRF |
| | 83158 | chain | acid | SGSSSGNTASLTITGAQAEDEADYYCNSRDSSGNHVVFGGGTKLTVL |
| | | vari- | se- | |
| | | able | quence | |
| | | region | of V_{L} | |
| | | | of CL- | |
| | | | 83158 | |
| 221 | CL- | Light | Nu- | tcttctgagctgactcaggaccctgctgtgtctgtggccttgggacagacagtcaggatca |
| | 83158 | chain | cleic | catgccaaggagacagcctcagaagctattatgcaagctggtaccagcagaagccag |
| | | vari- | acid | gacagacccctttacttgtcatctatggtaaaaacaaccggccctcagggatcccag |
| | | able | se- | accgattctctggctccagctcaggaaacacagcttccttgaccatcactggggctc |
| | | region | quence | aggcggaagatgaggctgactattactgtaactcccgggacagcagtggtaacc |
| | | | of V_{L} | atgtggtgttcggcggagggaccaagctgaccgtcctag |
| | | | of CL- | |
| | | | 83158 | |
| 222 | CL- | Full | Amino | SSELTQDPAVSVALGQTVRITCQGDSLRSYYASWYQQKPGQTPLLVIYGKNNRPSGIPDRFSGSS |
| | 83158 | light | acid | SGNTASLTITGAQAEDEADYYCNSRDSSGNHVVFGGGTKLTVLGQPKAAPSVTLFPPSSEELQAN |
| | | chain | se- | KATLVCLISDFYPGAVTVAWKADSSPVKAGVETTTPSKQSNNKYAASSYLSLTPEQWKSHRSYSC |
| | | se- | quence | QVTHEGSTVEKTVAPTECS |
| | | quence | of CL- | |
| | | | 83158 | |
| | | | light | |
| | | | chain | |
| 223 | CL- | Full | Nu- | tcttctgagctgactcaggaccctgctgtgtctgtggccttgggacagacagtcaggatca |
| | 83158 | light | cleic | catgccaaggagacagcctcagaagctattatgcaagctggtaccagcagaagccaggaca |
| | | chain | acid | gacccctttacttgtcatctatggtaaaaacaaccggccctcagggatcccagaccgattc |
| | | se- | se- | tctggctccagctcaggaaacacagcttccttgaccatcactggggctcaggcggaagatga |
| | | quence | quence | ggctgactattactgtaactcccgggacagcagtggtaaccatgtggtgttcggcggaggga |
| | | | of CL- | ccaagctgaccgtcctaggtcagcccaaggctgccccctcggtcactctgttcccgccctc |
| | | | 83158 | ctctgaggagcttcaagccaacaaggccacactggtgtgtctcataagtgacttctacccgg |
| | | | light | gagccgtgacagtggcctggaaggcagatagcagccccgtcaaggcgggagtggagac |
| | | | chain | caccacaccctccaaacaaagcaacaacaagtacgcggccagcagctatctgagcctgac |
| | | | | gcctgagcagtggaagtcccacagaagctacagctgccaggtcacgcatgaaggga |
| | | | | gcaccgtggagaagacagtggcccctacagaatgttca |
| 224 | CL- | Heavy | Amino | EVQLVESGGDLVQPGGSLRLSCAASGFTFYSYAMNWVRQAPGKGLKWVSGISGSGDITYYADSV |
| | 148712 | chain | acid | KGRFTISRDNSKNTLYLQMHSLRAEDTAVYYCAKGQLERRYYKYYGMDVWGQGTTVTVSS |
| | | vari- | se- | |
| | | able | quence | |
| | | region | of V_{H} | |
| | | | of CL- | |
| | | | 148712 | |
| 225 | CL- | Heavy | Nu- | gaggtgcagctggtggagtctgggggagacttagtacagcctggggggtccctgagactctct |
| | 148712 | chain | cleic | tgtgcagcctctggattcaccttttacagctatgccatgaactgggtccgccaggctccaggg |
| | | vari- | acid | aaggggctaaaatgggtctcaggtattagtggtagtggtgatatcacatactacgcagact |
| | | able | se- | ccgtgaagggccggttcaccatctccagagacaattccaagaacacgctgtatctgcaaatgc |
| | | region | quence | acagcctgagagccgaagacacggccgtatattactgtgcgaaaggccaactggaacgacggt |
| | | | of V_{H} | actacaagtactacggtatggacgtctggggccaagggaccacggtcaccgtctcctcag |
| | | | of CL- | |
| | | | 148712 | |
| 226 | CL- | Full | Amino | EVQLVESGGDLVQPGGSLRLSCAASGFTFYSYAMNWVRQAPGKGLKWVSGISGSGDITYYADSV |
| | 148712 | heavy | acid | KGRFTISRDNSKNTLYLQMHSLRAEDTAVYYCAKGQLERRYYKYYGMDVWGQGTTVTVSSASTK |
| | | chain | se- | GPSVFPLAPCSRSTSESTAALGCLVKDYFPEPVTVSWNSGALTSGVHTFPAVLQSSGLYSLSSVVT |
| | | se- | quence | VPSSSLGTKTYTCNVDHKPSNTKVDKRVESKYGPPCPPCPAPEFEGGPSVFLFPPKPKDTLMISRT |
| | | quence | of CL- | PEVTCVVVDVSQEDPEVQFNWYVDGVEVHNAKTKPREEQFNSTYRVVSVLTVLHQDWLNGKEY |
| | | | 148712 | KCKVSNKGLPSSIEKTISKAKGQPREPQVYTLPPSQEEMTKNQVSLTCLVKGFYPSDIAVEWESNG |
| | | | heavy | QPENNYKTTPPVLDSDGSFFLYSRLTVDKSRWQEGNVFSCSVMHEALHNHYTQKSLSLSLGK |
| | | | chain | |
| 227 | CL- | Full | Nu- | gaggtgcagctggtggagtctgggggagacttagtacagcctggggggtccctgagactctctt |
| | 148712 | heavy | cleic | gtgcagcctctggattcaccttttacagctatgccatgaactgggtccgccaggctccagggaa |
| | | chain | acid | ggggctaaaatgggtctcaggtattagtggtagtggtgatatcacatactacgcagactccgtg |
| | | se- | se- | aagggccggttcaccatctccagagacaattccaagaacacgctgtatctgcaaatgcacagcc |
| | | quence | quence | tgagagccgaagacacggccgtatattactgtgcgaaaggccaactggaacgacggtactacaa |
| | | | of CL- | gtactacggtatggacgtctggggccaagggaccacggtcaccgtctcctcagccagcaccaa |
| | | | 148712 | gggcccttccgtgttccccctggccccttgcagcaggagcacctccgaatccacagctgccct |
| | | | heavy | gggctgtctggtgaaggactactttcccgagcccgtgaccgtgagctggaacagcggcgctct |
| | | | chain | gacatccggcgtccacacctttcctgccgtcctgcagtcctccggcctctactccctgtcctc |
| | | | | cgtggtgaccgtgcctagctcctccctcggcaccaagacctacacctgtaacgtggaccacaa |
| | | | | accctccaacaccaaggtggacaaacgggtcgagagcaagtacggccctccctgccctccttg |
| | | | | tcctgcccccgagttcgaaggcggacccagcgtgttcctgttccctcctaagcccaaggacac |
| | | | | cctcatgatcagccggacacccgaggtgacctgcgtggtggtggatgtgagccaggaggacc |
| | | | | ctgaggtccagttcaactggtatgtggatggcgtggaggtgcacaacgccaagacaaagcccc |
| | | | | gggaagagcagttcaactccacctacagggtggtcagcgtgctgaccgtgctgcatcaggact |
| | | | | ggctgaacggcaaggagtacaagtgcaaggtcagcaataagggactgcccagcagcatcga |
| | | | | gaagaccatctccaaggctaaaggccagccccgggaacctcaggtgtacaccctgcctccca |
| | | | | gccaggaggagatgaccaagaaccaggtgagcctgacctgcctggtgaagggattctaccct |
| | | | | tccgacatcgccgtggagtgggagtccaacggccagcccgagaacaattataagaccacccc |
| | | | | tcccgtcctcgacagcgacggatccttctttctgtactccaggctgaccgtgg |
| | | | | ataagtccaggtggcaggaaggcaacgtgttcagctgctccgtga |
| | | | | tgcacgaggccctgcacaatcactacacccagaagtccctgagcctgtccctgggaaag |
| 228 | CL- | Light | Amino | DIQMTQSPSSLSASVGDRVTITCRANQGIRNDLGWYQQKPGKAPKRLIYGASSLKSGVPSRFSGS |
| | 148712 | chain | acid | GSGTEFTLTISSLQPEDFATYYCLQQNSYPLTFGGGTKVEIK |
| | | vari- | se- | |
| | | able | quence | |
| | | region | of V_{L} | |
| | | | of CL- | |
| | | | 148712 | |
| 229 | CL- | Light | Nu- | gacatccagatgacccagtctccatcctccctgtctgcatctgtaggagacagagtcaccatca |
| | 148712 | chain | cleic | cttgccgggcaaatcagggcattagaaatgatttaggctggtatcagcagaaaccaggga |
| | | vari- | acid | aagcccctaagcgcctgatctatggtgcatccagtttgaaaagtggggtcccatcaagg |
| | | able | se- | ttcagcggcagtggatctgggacagaattcactctcacaatcagcagcctgcagcctgaa |
| | | region | quence | gattttgcaacctactattgtctacagcagaatagttacccgctcactttcggcggaggga |
| | | | of V_{L} | ccaaggtggagatcaaac |
| | | | of CL- | |
| | | | 148712 | |
| 230 | CL- | Full | Amino | DIQMTQSPSSLSASVGDRVTITCRANQGIRNDLGWYQQKPGKAPKRLIYGASSLKSGVPSRFSGS |
| | 148712 | light | acid | GSGTEFTLTISSLQPEDFATYYCLQQNSYPLTFGGGTKVEIKRTVAAPSVFIFPPSDEQLKSGTA |
| | | chain | se- | SVVCLLNNFYPREAKVQWKVDNALQSGNSQESVTEQDSKDSTYSLSSTLTLSKADYEKHKVYACE |
| | | se- | quence | VTHQGLSSPVTKSFNRGEC |
| | | quence | of CL- | |
| | | | 148712 | |
| | | | light | |
| | | | chain | |
| 231 | CL- | Full | Nu- | gacatccagatgacccagtctccatcctccctgtctgcatctgtaggagacagagtcaccatca |
| | 148712 | light | cleic | cttgccgggcaaatcagggcattagaaatgatttaggctggtatcagcagaaaccagggaa |
| | | chain | acid | agcccctaagcgcctgatctatggtgcatccagtttgaaaagtggggtcccatcaaggttca |
| | | se- | se- | gcggcagtggatctgggacagaattcactctcacaatcagcagcctgcagcctgaagatttt |
| | | quence | quence | gcaacctactattgtctacagcagaatagttacccgctcactttcggcggagggaccaaggt |
| | | | of CL- | ggagatcaaacgtacggtggccgctccctccgtgttcatcttcccaccttccgacgagc |
| | | | 148712 | agctgaagtccggcaccgcttctgtcgtgtgcctgctgaacaacttctacccccgcgaggcc |
| | | | light | aaggtgcagtggaaggtggacaacgccctgcagtccggcaactcccaggaatccgtg |
| | | | chain | accgagcaggactccaaggacagcacctactccctgtcctccaccctgaccctgtccaag |
| | | | | gccgactacgagaagcacaaggtgtacgcctgcgaagtgacccaccagggcctgtc |
| | | | | tagccccgtgaccaagtctttcaaccggggcgagtgt |
| 232 | CL- | Heavy | Amino | AVQLVESGGGLVQPGGSLKLSCAASGFTFSGSTMHWVRQASGRGLEWVGRIRSKADSYATAYSA |
| | 148729 | chain | acid | SVKGRFTISRDDSKKMAYLQMNSLKTEDMAVYYCTRSGDYWGQGTLVTVSS |
| | | vari- | se- | |
| | | able | quence | |
| | | region | of V_{H} | |
| | | | of CL- | |
| | | | 148729 | |
| 233 | CL- | Heavy | Nu- | gcggtgcagctggtggagtccgggggaggcttggtccagcctggggggtccctgaaactct |
| | 148729 | chain | cleic | cctgtgcagcctctggattcaccttcagtggctctactatgcactggtccgccaggc |
| | | vari- | acid | ttccgggagagggctggagtgggttggccgtattagaagcaaagctgacagttacgc |
| | | able | se- | gacagcatatagtgcgtcggtgaaaggcaggttcaccatctccagagatgattcaaa |
| | | region | quence | gaaaatggcgtatctgcaaatgaacagcctgaaaaccgaggacatggccgt |
| | | | of V_{H} | gtattattgtactagaagcggggactactggggccaggga |
| | | | of CL- | accctggtcaccgtctcctcag |
| | | | 148729 | |
| 234 | CL- | Full | Amino | AVQLVESGGGLVQPGGSLKLSCAASGFTFSGSTMHWVRQASGRGLEWVGRIRSKADSYATAYSA |
| | 148729 | heavy | acid | SVKGRFTISRDDSKKMAYLQMNSLKTEDMAVYYCTRSGDYWGQGTLVTVSSASTKGPSVFPLAP |
| | | chain | se- | CSRSTSESTAALGCLVKDYFPEPVTVSWNSGALTSGVHTFPAVLQSSGLYSLSSVVTVPSSSLGTK |
| | | se- | quence | TYTCNVDHKPSNTKVDKRVESKYGPPCPPCPAPEFEGGPSVFLFPPKPKDTLMISRTPEVTCVVVD |
| | | quence | of CL- | VSQEDPEVQFNWYVDGVEVHNAKTKPREEQFNSTYRVVSVLTVLHQDWLNGKEYKCKVSNKGL |
| | | | 148729 | PSSIEKTISKAKGQPREPQVYTLPPSQEEMTKNQVSLTCLVKGFYPSDIAVEWESNGQPENNYKT |
| | | | heavy | TPPVLDSDGSFFLYSRLTVDKSRWQEGNVFSCSVMHEALHNHYTQKSLSLSLGK |
| | | | chain | |
| 235 | CL- | Full | Nu- | gcggtgcagctggtggagtccgggggaggcttggtccagcctggggggtccctgaaactct |
| | 148729 | heavy | cleic | cctgtgcagcctctggattcaccttcagtggctctactatgcactggtccgccaggcttcc |
| | | chain | acid | gggagagggctggagtgggttggccgtattagaagcaaagctgacagttacgcgacagcata |
| | | se- | se- | tagtgcgtcggtgaaaggcaggttcaccatctccagagatgattcaaagaaaatggcgtatct |
| | | quence | quence | gcaaatgaacagcctgaaaaccgaggacatggccgtgtattattgtactagaagcggggacta |
| | | | of CL- | ctggggccagggaaccctggtcaccgtctcctcagccagcaccaagggcccttccgtgttc |
| | | | 148729 | cccctggccccttgcagcaggagcacctccgaatccacagctgccctgggctgtctggtgaag |
| | | | heavy | gactactttcccgagcccgtgaccgtgagctggaacagcggcgctctgacatccggcgtcc |
| | | | chain | acacctttcctgccgtcctgcagtcctccggcctctactccctgtcctccgtggtgaccgtg |
| | | | | cctagctcctccctcggcaccaagacctacacctgtaacgtggaccacaaaccctccaacacc |
| | | | | aaggtggacaaacgggtcgagagcaagtacggccctccctgccctccttgtcctgcccccgag |
| | | | | ttcgaaggcggacccagcgtgttcctgttccctcctaagcccaaggacaccctcatgatc |
| | | | | agccggacacccgaggtgacctgcgtggtggtggatgtgagccaggaggaccctgaggtccag |
| | | | | ttcaactggtatgtggatggcgtggaggtgcacaacgccaagacaaagccccgggaagagcag |
| | | | | ttcaactccacctacagggtggtcagcgtgctgaccgtgctgcatcaggactggctgaacggc |
| | | | | aaggagtacaagtgcaaggtcagcaataagggactgcccagcagcatcgagaagaccatctccaa |
| | | | | ggctaaaggccagccccgggaacctcaggtgtacaccctgcctcccagccaggagg |
| | | | | agatgaccaagaaccaggtgagcctgacctgcctggtgaagggattctacccttccgacatc |
| | | | | gccgtggagtgggagtccaacggccagcccgagaacaattataagac |
| | | | | cacccctcccgtcctcgacagcgacggatccttctttctgtactccaggctga |
| | | | | ccgtggataagtccaggtggcaggaaggcaacgtgttcagctgctccgtgatgcacgaggc |
| | | | | cctgcacaatcactacacccagaagtccctgagcctgtccctgggaaag |
| 236 | CL- | Light | Amino | QSVLTQPPSVSAAPGQKVTISCSGSSSNIGKNYVAWYQQLPGTAPKLLIYDTNKRPSGIPDRFSGS |
| | 148729 | chain | acid | KSGTSATLDITGLQTGDEADYYCGTWDISLSVGWLFGGGTKVTVL |
| | | vari- | se- | |
| | | able | quence | |
| | | region | of V_{L} | |
| | | | of CL- | |
| | | | 148729 | |
| 237 | CL- | Light | Nu- | cagtctgtgttgacgcagccgccctcagtgtctgcggccccaggacagaaggtcaccatctcct |
| | 148729 | chain | cleic | gttctggaagcagctccaacattggaaaaaattatgtagcctggtaccagcaactcccagga |
| | | vari- | acid | acagcccccaaactcctcatttatgacactaataagcgaccctcagggattc |
| | | able | se- | ctgaccgattctctggctccaagtctggcacgtcagccaccctggacatcaccggactc |
| | | region | quence | cagactggggacgaggccgattattactgcggaacatgggatatcagcctgagtgttgg |
| | | | of V_{L} | ttggctgttcggcggagggaccaaggtgaccgtcctag |
| | | | of CL- | |
| | | | 148729 | |
| 238 | CL- | Full | Amino | QSVLTQPPSVSAAPGQKVTISCSGSSSNIGKNYVAWYQQLPGTAPKLLIYDTNKRPSGIPDRFSGS |
| | 148729 | light | acid | KSGTSATLDITGLQTGDEADYYCGTWDISLSVGWLFGGGTKVTVLGQPKAAPSVTLFPPSSEELQ |
| | | chain | se- | ANKATLVCLISDFYPGAVTVAWKADSSPVKAGVETTTPSKQSNNKYAASSYLSLTPEQWKSHKSY |
| | | se- | quence | SCQVTHEGSTVEKTVAPTECS |
| | | quence | of CL- | |
| | | | 148729 | |
| | | | light | |
| | | | chain | |
| 239 | CL- | Full | Nu- | cagtctgtgttgacgcagccgccctcagtgtctgcggccccaggacagaaggtcaccatctcct |
| | 148729 | light | cleic | gttctggaagcagctccaacattggaaaaaattatgtagcctggtaccagcaactcccag |
| | | chain | acid | gaacagcccccaaactcctcatttatgacactaataagcgaccctcagggattcctgaccg |
| | | se- | se- | attctctggctccaagtctggcacgtcagccaccctggacatcaccggactccagactggg |
| | | quence | quence | gacgaggccgattattactgcggaacatgggatatcagcctgagtgttggttggctgttcg |
| | | | of CL- | gcggagggaccaaggtgaccgtcctaggccagcctaaggccgctccttccgttacactgtt |
| | | | 148729 | ccctccatcctccgaggaactgcaggccaacaaggctaccctcgtgtgcctgatctccga |
| | | | light | cttttaccctggcgctgtgaccgtggcctggaaggctgatagttctcctgtgaaggccggc |
| | | | chain | gtggaaaccaccacaccttccaagcagtccaacaacaaatacgccgcctcctcctacctgt |
| | | | | ctctgacccctgaacagtggaagtcccacaagtcctacagctgccaagtgacc |
| | | | | cacgagggctccaccgtggaaaagacagtggctcctaccgagtgctcca |
| 240 | Human | IGHG1 | Human | gcctccaccaagggcccatcggtcttccccctggcaccctcctccaagagcacctctgggg |
| | IgG1 | *01 | Heavy | gcacagcggccctgggctgcctggtcaaggactacttccccgaaccggtgacggtgtcgtg |
| | con- | | Chain | gaactcaggcgccctgaccagcggcgtgcacaccttcccggctgtcctacagtcctcagg |
| | stant | | Con- | actctactccctcagcagcgtggtgaccgtgccctccagcagcttgggcacccagacctacat |
| | region | | stant | ctgcaacgtgaatcacaagcccagcaacaccaaggtggacaagaaagttgagcccaaatctt |
| | | | Region | gtgacaaaactcacacatgcccaccgtgcccagcacctgaactcctggggggaccgtcagtc |
| | | | (IGHG1 | ttcctcttccccccaaaacccaaggacaccctcatgatctcccggacccctgaggtc |
| | | | *01) | acatgcgtggtggtggacgtgagccacgaagaccctgaggtcaagttcaactggtacgtgg |
| | | | Nucle- | acggcgtggaggtgcataatgccaagacaaagccgcgggaggagcagtacaacagcacgta |
| | | | otide | ccgggtggtcagcgtcctcaccgtcctgcaccaggactggctgaatggcaaggagtacaagt |
| | | | Se- | gcaaggtctccaacaaagccctcccagcccccatcgagaaaaccatctccaaagccaa |
| | | | quence | agggcagccccgagaaccacaggtgtacaccctgcccccatcccgggatgagctgac |
| | | | | caagaaccaggtcagcctgacctgcctggtcaaaggcttctatcccagcgacatcgccg |
| | | | | tggagtgggagagcaatgggcagccggagaacaactacaagaccacgcct |
| | | | | cccgtgctggactccgacggctccttcttcctctacagcaagctcaccgtgga |
| | | | | caagagcaggtggcagcaggggaacgtcttctcatgctccgtgatgcatgaggctct |
| | | | | gcacaaccactacacgcagaagagcctctccctgtctccgggtaaa |
| 241 | | | Human | ASTKGPSVFPLAPSSKSTSGGTAALGCLVKDYFPEPVTVSWNSGALTSGVHTFPAVLQSSGLYSLS |
| | | | Heavy | SVVTVPSSSLGTQTYICNVNHKPSNTKVDKKVEPKSCDKTHTCPPCPAPELLGGPSVFLFPPKPKD |
| | | | Chain | TLMISRTPEVTCVVVDVSHEDPEVKFNWYVDGVEVHNAKTKPREEQYNSTYRVVSVLTVLHQDW |
| | | | Con- | LNGKEYKCKVSNKALPAPIEKTISKAKGQPREPQVYTLPPSRDELTKNQVSLTCLVKGFYPSDIAVE |
| | | | stant | WESNGQPENNYKTTPPVLDSDGSFFLYSKLTVDKSRWQQGNVFSCSVMHEALHNHYTQKSLSLS |
| | | | Region | PGK |
| | | | (IGHG1 | |
| | | | *01) | |
| | | | Pro- | |
| | | | tein | |
| | | | Se- | |
| | | | quence | |
| | | | (P0 | |
| | | | 1857) | |
| 242 | Human | IGHG1 | Human | gcctccaccaagggcccatcggtcttccccctggcaccctcctccaagagcacctctgggg |
| | IgG1 | *02 | Heavy | gcacagcggccctgggctgcctggtcaaggactacttccccgaaccggtgacggtgtcgtg |
| | con- | or | Chain | gaactcaggcgccctgaccagcggcgtgcacaccttcccggctgtcctacagtcctcaggac |
| | stant | IGHG1 | Con- | tctactccctcagcagcgtggtgaccgtgccctccagcagcttgggcacccagacctacat |
| | region | *05 | stant | ctgcaacgtgaatcacaagcccagcaacaccaaggtggacaagaaagttgagcccaaatct |
| | | | Region | tgtgacaaaactcacacatgcccaccgtgcccagcacctgaactcctggggggaccgtcag |
| | | | (IGHG1 | tcttcctcttccccccaaaacccaaggacaccctcatgatctcccggacccctgaggtcaca |
| | | | *02 | tgcgtggtggtggacgtgagccacgaagaccctgaggtcaagttcaactggtacgtggacgg |
| | | | or | cgtggaggtgcataatgccaagacaaagccgcgggaggagcagtacaacagcacgtaccgtg |
| | | | IGHG1 | tggtcagcgtcctcaccgtcctgcaccaggactggctgaatggcaaggagtacaagtgcaag |
| | | | *05) | gtctccaacaaagccctcccagcccccatcgagaaaaccatctccaaagccaaagggcagcc |
| | | | Nucle- | ccgagaaccacaggtgtacaccctgcccccatcccgggatgagctgaccaagaaccaggtca |
| | | | otide | gcctgacctgcctggtcaaaggcttctatcccagcgacatcgccgtggagtgggagagcaat |
| | | | Se- | gggcagccggagaacaactacaagaccacgcctcccgtgctggactccgacggctccttctt |
| | | | quence | cctctacagcaagctcaccgtggacaagagcaggtggcagcaggggaacgtcttctcatgct |
| | | | | ccgtgatgcatgaggctctgcacaaccactacacgcagaagagcctctccctgtctccgggt |
| | | | | aaa |
| 243 | | | Human | ASTKGPSVFPLAPSSKSTSGGTAALGCLVKDYFPEPVTVSWNSGALTSGVHTFPAVLQSSGLYSLS |
| | | | Heavy | SVVTVPSSSLGTQTYICNVNHKPSNTKVDKKVEPKSCDKTHTCPPCPAPELLGGPSVFLFPPKPKD |
| | | | Chain | TLMISRTPEVTCVVVDVSHEDPEVKFNWYVDGVEVHNAKTKPREEQYNSTYRVVSVLTVLHQDW |
| | | | Constant | LNGKEYKCKVSNKALPAPIEKTISKAKGQPREPQVYTLPPSRDELTKNQVSLTCLVKGFYPSDIAV |
| | | | Region | EWESNGQPENNYKTTPPVLDSDGSFFLYSKLTVDKSRWQQGNVFSCSVMHEALHNHYTQKSLSLS |
| | | | (IGHG1*02) | PGK |
| | | | Protein | |
| | | | Sequence | |
| 244 | Human | IGHG1 | Human | gcctccaccaagggcccatcggtcttccccctggcaccctcctccaagagcacctctgggggca |
| | IgG1 | *03 | Heavy | cagcggccctgggctgcctggtcaaggactacttccccgaaccggtgacggtgtcgtggaact |
| | con- | | Chain | caggcgccctgaccagcggcgtgcacaccttcccggctgtcctacagtcctcaggactctact |
| | stant | | Constant | ccctcagcagcgtggtgaccgtgccctccagcagcttgggcacccagacctacatctgcaacg |
| | region | | Region | tgaatcacaagcccagcaacaccaaggtggacaagagagttgagcccaaatcttgtgacaaaa |
| | | | (IGHG1*03) | ctcacacatgcccaccgtgcccagcacctgaactcctggggggaccgtcagtcttcctcttcc |
| | | | Nucleotide | ccccaaaacccaaggacaccctcatgatctcccggacccctgaggtcacatgcgtggtggtgg |
| | | | Sequence | acgtgagccacgaagaccctgaggtcaagttcaactggtacgtggacggcgtggaggtgcata |
| | | | (Y14737) | atgccaagacaaagccgcgggaggagcagtacaacagcacgtaccgtgtggtcagcgtcct |
| | | | | caccgtcctgcaccaggactggctgaatggcaaggagtacaagtgcaaggtctccaacaaagc |
| | | | | cctcccagcccccatcgagaaaaccatctccaaagccaaagggcagccccgagaaccacaggt |
| | | | | gtacaccctgcccccatcccgggaggagatgaccaagaaccaggtcagcctgacctgcct |
| | | | | ggtcaaaggcttctatcccagcgacatcgccgtggagtgggagagcaatgggcagccgga |
| | | | | gaacaactacaagaccacgcctcccgtgctggactccgacggctccttcttcctctatagca |
| | | | | agctcaccgtggacaagagcaggtggcagcaggggaacgtcttctcatgctccgtgatgcat |
| | | | | gaggctctgcacaaccactacacgcagaagagcctctccctgtccccgggtaaa |
| 245 | | | Human | ASTKGPSVFPLAPSSKSTSGGTAALGCLVKDYFPEPVTVSWNSGALTSGVHTFPAVLQSSGLYSLS |
| | | | Heavy | SVVTVPSSSLGTQTYICNVNHKPSNTKVDKRVEPKSCDKTHTCPPCPAPELLGGPSVFLFPPKPKD |
| | | | Chain | TLMISRTPEVTCVVVDVSHEDPEVKFNWYVDGVEVHNAKTKPREEQYNSTYRVVSVLTVLHQDW |
| | | | Constant | LNGKEYKCKVSNKALPAPIEKTISKAKGQPREPQVYTLPPSREEMTKNQVSLTCLVKGFYPSDIAV |
| | | | Region | EWESNGQPENNYKTTPPVLDSDGSFFLYSKLTVDKSRWQQGNVFSCSVMHEALHNHYTQKSLSLS |
| | | | (IGHG1*03) | PGK |
| | | | Protein | |
| | | | Sequence | |
| 246 | Human | IGHG1 | Human | gcctccaccaagggcccatcggtcttccccctggcaccctcctccaagagcacctctggggg |
| | IgG1 | *04 | Heavy | cacagcggccctgggctgcctggtcaaggactacttccccgaaccggtgacggtgtcgtgg |
| | con- | | Chain | aactcaggcgccctgaccagcggcgtgcacaccttcccggctgtcctacagtcctcagga |
| | stant | | Constant | ctctactccctcagcagcgtggtgaccgtgccctccagcagcttgggcacccagacctacat |
| | region | | Region | ctgcaacgtgaatcacaagcccagcaacaccaaggtggacaagaaagttgagcccaaatctt |
| | | | (IGHG1*04) | gtgacaaaactcacacatgcccaccgtgcccagcacctgaactcctggggggaccgtcagt |
| | | | Nucleotide | cttcctcttccccccaaaacccaaggacaccctcatgatctcccggacccctgaggtcac |
| | | | Sequence | atgcgtggtggtggacgtgagccacgaagaccctgaggtcaagttcaactggtacgtgg |
| | | | | acggcgtggaggtgcataatgccaagacaaagccgcgggaggagcagtacaacagcacgt |
| | | | | accgtgtggtcagcgtcctcaccgtcctgcaccaggactggctgaatggcaaggagta |
| | | | | caagtgcaaggtctccaacaaagccctcccagcccccatcgagaaaaccatctccaa |
| | | | | agccaaagggcagccccgagaaccacaggtgtacaccctgcccccatcccgggatgagc |
| | | | | tgaccaagaaccaggtcagcctgacctgcctggtcaaaggcttctatcccagcgacatc |
| | | | | gccgtggagtgggagagcaatgggcagccggagaacaactacaagaccacgcctc |
| | | | | ccgtgctggactccgacggctccttcttcctctacagcaagctcaccgtgga |
| | | | | caagagcaggtggcagcaggggaacatcttctcatgctccgtgatgcatgaggctctg |
| | | | | cacaaccactacacgcagaagagcctctccctgtctccgggtaaa |
| 247 | | | Human | ASTKGPSVFPLAPSSKSTSGGTAALGCLVKDYFPEPVTVSWNSGALTSGVHTFPAVLQSSGLYSLS |
| | | | Heavy | SVVTVPSSSLGTQTYICNVNHKPSNTKVDKKVEPKSCDKTHTCPPCPAPELLGGPSVFLFPPKPKD |
| | | | Chain | TLMISRTPEVTCVVVDVSHEDPEVKFNWYVDGVEVHNAKTKPREEQYNSTYRVVSVLTVLHQDW |
| | | | Constant | LNGKEYKCKVSNKALPAPIEKTISKAKGQPREPQVYTLPPSRDELTKNQVSLTCLVKGFYPSDIAV |
| | | | Region | EWESNGQPENNYKTTPPVLDSDGSFFLYSKLTVDKSRWQQGNIFSCSVMHEALHNHYTQKSLSLS |
| | | | (IGHG1*04) | PGK |
| | | | Protein | |
| | | | Sequence | |
| 248 | Dis- | Dis- | Disabled | gcctccaccaagggcccatcggtcttccccctggcaccctcctccaagagcacctctgg |
| | abled | abled | Human | gggcacagcggccctgggctgcctggtcaaggactacttccccgaaccggtgacggtg |
| | Human | human | IGHG1*01 | tcgtggaactcaggcgccctgaccagcggcgtgcacaccttcccggctgtcctacagt |
| | IgG1 | IGHG1 | Heavy | cctcaggactctactccctcagcagcgtggtgaccgtgccctccagcagcttgggcacc |
| | heavy | *01 | Chain | cagacctacatctgcaacgtgaatcacaagcccagcaacaccaaggtggacaagaaa |
| | chain | | Constant | gtggagcccaaatcttgtgacaaaactcacacatgcccaccgtgcccagcacctgaac |
| | con- | | Region | tcgcgggggcaccgtcagtcttcctcttccccccaaaacccaaggacaccctcatga |
| | stant | | Nucleotide | tctcccggacccctgaggtcacatgcgtggtggtggacgtgagccacgaagaccctga |
| | region | | Sequence. | ggtcaagttcaactggtacgtggacggcgtggaggtgcataatgccaagacaaagcc |
| | | | | gcgggaggagcagtacaacagcacgtaccgtgtggtcagcgtcctcaccgtcct |
| | | | | gcaccaggactggctgaatggcaaggagtacaagtgcaaggtctccaacaaagccc |
| | | | | tcccagcccccatcgagaaaaccatctccaaagccaaagggcagccccgag |
| | | | | aaccacaggtgtacaccctgcccccatcccgggatgagctgaccaagaac |
| | | | | caggtcagcctgacctgcctggtcaaaggcttctatcccagcgacatcgccgtgg |
| | | | | agtgggagagcaatgggcagccggagaacaactacaagaccacgcctcc |
| | | | | cgtgctggactccgacggctccttcttcctctacagcaagctcaccgtgga |
| | | | | caagagcaggtggcagcaggggaacgtcttctcatgctccgtgatgcatgaggct |
| | | | | ctgcacaaccactacacgcagaagagcctctccctgtctccgggtaaa |
| 249 | | | Disabled | ASTKGPSVFPLAPSSKSTSGGTAALGCLVKDYFPEPVTVSWNSGALTSGVHTFPAVLQSSGLYSLS |
| | | | Human | SVVTVPSSSLGTQTYICNVNHKPSNTKVDKKVEPKSCDKTHTCPPCPAPELAGAPSVFLFPPKPKD |
| | | | IGHG1*01 | TLMISRTPEVTCVVVDVSHEDPEVKFNWYVDGVEVHNAKTKPREEQYNSTYRVVSVLTVLHQDW |
| | | | Heavy | LNGKEYKCKVSNKALPAPIEKTISKAKGQPREPQVYTLPPSRDELTKNQVSLTCLVKGFYPSDIAV |
| | | | Chain | EWESNGQPENNYKTTPPVLDSDGSFFLYSKLTVDKSRWQQGNVFSCSVMHEALHNHYTQKSLSLS |
| | | | Constant | PGK |
| | | | Region | |
| | | | Amino | |
| | | | Acid | |
| | | | Sequence. | |
| | | | Two | |
| | | | residues | |
| | | | that | |
| | | | differ | |
| | | | from the | |
| | | | wild-type | |
| | | | sequence | |
| | | | are iden- | |
| | | | tified | |
| | | | in bold. | |
| 250 | Human | IGHG2 | Human | gcctccaccaagggcccatcggtcttccccctggcgccctgctccaggagcacct |
| | IgG2 | *01 | Heavy | ccgagagcacagccgccctgggctgcctggtcaaggactacttccccgaaccgg |
| | con- | or | Chain | tgacggtgtcgtggaactcaggcgctctgaccagcggcgtgcacaccttccca |
| | stant | IGHG2 | Constant | gctgtcctacagtcctcaggactctactccctcagcagcgtggtgaccgtgccc |
| | region | *04 | Region | tccagcaacttcggcacccagacctacacctgcaacgtagatcacaagcccagca |
| | | or | (IGHG2*01 | acaccaaggtggacaagacagttgagcgcaaatgttgtgtcgagtgcccaccgt |
| | | IGHG2 | or | gcccagcaccacctgtggcaggaccgtcagtcttcctcttccccccaaaaccca |
| | | *05 | IGHG2*03 | aggacaccctcatgatctcccggacccctgaggtcacgtgcgtggtggtggacg |
| | | | or | tgagccacgaagaccccgaggtccagttcaactggtacgtggacggcgtggagg |
| | | | IGHG2*05) | tgcataatgccaagacaaagccacgggaggagcagttcaacagcacgttccgtg |
| | | | Nucleotide | tggtcagcgtcctcaccgttgtgcaccaggactggctgaacggcaaggagtaca |
| | | | Sequence | agtgcaaggtctccaacaaaggcctcccagcccccatcgagaaaaccatctcca |
| | | | | aaaccaaagggcagccccgagaaccacaggtgtacaccctgcccccatcccggg |
| | | | | aggagatgaccaagaaccaggtcagcctgacctgcctggtcaaaggcttctacc |
| | | | | ccagcgacatcgccgtggagtgggagagcaatgggcagccggagaacaactaca |
| | | | | agaccacacctcccatgctggactccgacggctccttcttcctctacagcaagc |
| | | | | tcaccgtggacaagagcaggtggcagcaggggaacgtcttctcatgctccgtga |
| | | | | tgcatgaggctctgcacaaccactacacgcagaagagcctctccctgtctccgggtaaa |
| 251 | | | Human | ASTKGPSVFPLAPCSRSTSESTAALGCLVKDYFPEPVTVSWNSGALTSGVHTFPAVLQSSGLYSLS |
| | | | Heavy | SVVTVPSSNFGTQTYTCNVDHKPSNTKVDKTVERKCCVECPPCPAPPVAGPSVFLFPPKPKDTLMI |
| | | | Chain | SRTPEVTCVVVDVSHEDPEVQFNWYVDGVEVHNAKTKPREEQFNSTFRVVSVLTVVHQDWLNG |
| | | | Constant | KEYKCKVSNKGLPAPIEKTISKTKGQPREPQVYTLPPSREEMTKNQVSLTCLVKGFYPSDIAVEWE |
| | | | Region | SNGQPENNYKTTPPMLDSDGSFFLYSKLTVDKSRWQQGNVFSCSVMHEALHNHYTQKSLSLSPG |
| | | | (IGHG2*01) | K |
| | | | Protein | |
| | | | Sequence | |
| 252 | Human | IGHG2 | Human | gcctccaccaagggcccatcggtcttccccctggcgccctgctccaggagcacc |
| | IgG2 | *02 | Heavy | tccgagagcacagcggccctgggctgcctggtcaaggactacttccccgaacc |
| | con- | | Chain | ggtgacggtgtcgtggaactcaggcgctctgaccagcggcgtgcacaccttcccg |
| | stant | | Constant | gctgtcctacagtcctcaggactctactccctcagcagcgtggtgaccgtgacct |
| | region | | Region | ccagcaacttcggcacccagacctacacctgcaacgtagatcacaagcccagcaa |
| | | | (IGHG2*02) | caccaaggtggacaagacagttgagcgcaaatgttgtgtcgagtgcccaccgt |
| | | | Nucleotide | gcccagcaccacctgtggcaggaccgtcagtcttcctcttccccccaaaacccaat |
| | | | Sequence | ggacaccctcatgatctcccggaccccgaggtcacgtgcgtggtggtggacgtga |
| | | | | gccacgaagaccccgaggtccagttcaactggtacgtggacggcatggaggtgca |
| | | | | taatgccaagacaaagccacgggaggagcagttcaacagcacgttccgtgtggtc |
| | | | | agcgtcctcaccgtcgtgcaccaggactggctgaacggcaaggagtacaagtgc |
| | | | | aaggtctccaacaaaggcctcccagcccccatcgagaaaaccatctccaaaac |
| | | | | caaagggcagccccgagaaccacaggtgtacaccctgcccccatcccgggagga |
| | | | | gatgaccaagaaccaggtcagcctgacctgcctggtcaaaggcttctaccccagc |
| | | | | gacatcgccgtggagtgggagagcaatgggcagccggagaacaactacaagac |
| | | | | cacacctcccatgctggactccgacggctccttcttcctctacagcaagctcacc |
| | | | | gtggacaagagcaggtggcagcaggggaacgtcttctcatgctccgtgatgcatgag |
| | | | | gctctgcacaaccactacacacagaagagcctctccctgtctccgggtaaa |
| 253 | | | Human | ASTKGPSVFPLAPCSRSTSESTAALGCLVKDYFPEPVTVSWNSGALTSGVHTFPAVLQSSGLYSLS |
| | | | Heavy | SVVTVTSSNFGTQTYTCNVDHKPSNTKVDKTVERKCCVECPPCPAPPVAGPSVFLFPPKPKDTLMI |
| | | | Chain | SRTPEVTCVVVDVSHEDPEVQFNWYVDGMEVHNAKTKPREEQFNSTFRVVSVLTVVHQDWLNG |
| | | | Constant | KEYKCKVSNKGLPAPIEKTISKTKGQPREPQVYTLPPSREEMTKNQVSLTCLVKGFYPSDIAVEWE |
| | | | Region | SNGQPENNYKTTPPMLDSDGSFFLYSKLTVDKSRWQQGNVFSCSVMHEALHNHYTQKSLSLSPG |
| | | | (IGHG2*02) | K |
| | | | Protein | |
| | | | Sequence | |
| 254 | Human | IGHG2 | Human | gcctccaccaagggcccatcggtcttccccctggcgccctgctccaggagcacct |
| | IgG2 | *04 | Heavy | ccgagagcacagcggccctgggctgcctggtcaaggactacttccccgaaccggtg |
| | con- | | Chain | acggtgtcgtggaactcaggcgctctgaccagcggcgtgcacaccttccca |
| | stant | | Constant | gctgtcctacagtcctcaggactctactccctcagcagcgtggtgaccgtgccct |
| | region | | Region | ccagcagcttgggcacccagacctacacctgcaacgtagatcacaagcccagcaa |
| | | | (IGHG2*04) | caccaaggtggacaagacagttgagcgcaaatgttgtgtcgagtgcccaccg |
| | | | Nucleotide | tgcccagcaccacctgtggcaggaccgtcagtcttcctcttccccccaaaaccca |
| | | | Sequence | aggacaccctcatgatctcccggacccctgaggtcacgtgcgtggtggtggac |
| | | | | gtgagccacgaagaccccgaggtccagttcaactggtacgtggacggcgtggag |
| | | | | gtgcataatgccaagacaaagccacgggaggagcagttcaacagcacgttccgtg |
| | | | | tggtcagcgtcctcaccgttgtgcaccaggactggctgaacggcaaggagtaca |
| | | | | agtgcaaggtctccaacaaaggcctcccagcccccatcgagaaaaccatct |
| | | | | ccaaaaccaaagggcagccccgagaaccacaggtgtacaccctgcccccatcccg |
| | | | | ggaggagatgaccaagaaccaggtcagcctgacctgcctggtcaaaggc |
| | | | | ttctaccccagcgacatcgccgtggagtgggagagcaatgggcagccggagaac |
| | | | | aactacaagaccacacctcccatgctggactccgacggctccttcttcctctac |
| | | | | agcaagctcaccgtggacaagagcaggtggcagcaggggaacgtcttctcatg |
| | | | | ctccgtgatgcatgaggctctgcacaaccactacacgcagaagagcctctccctgtc |
| | | | | tccgggtaaa |
| 255 | | | Human | ASTKGPSVFPLAPCSRSTSESTAALGCLVKDYFPEPVTVSWNSGALTSGVHTFPAVLQSSGLYSLS |
| | | | Heavy | SVVTVPSSSLGTQTYTCNVDHKPSNTKVDKTVERKCCVECPPCPAPPVAGPSVFLFPPKPKDTLMI |
| | | | Chain | SRTPEVTCVVVDVSHEDPEVQFNWYVDGVEVHNAKTKPREEQFNSTFRVVSVLTVVHQDWLNG |
| | | | Constant | KEYKCKVSNKGLPAPIEKTISKTKGQPREPQVYTLPPSREEMTKNQVSLTCLVKGFYPSDIAVEWE |
| | | | Region | SNGQPENNYKTTPPMLDSDGSFFLYSKLTVDKSRWQQGNVFSCSVMHEALHNHYTQKSLSLSPG |
| | | | (IGHG2*04) | K |
| | | | Protein | |
| | | | Sequence | |
| 256 | Human | IGHG2 | Human | gcctccaccaagggcccatcggtcttccccctggcgccctgctccaggagcacctccga |
| | IgG2 | *06 | Heavy | gagcacagcggccctgggctgcctggtcaaggactacttccccgaacc |
| | con- | | Chain | ggtgacggtgtcgtggaactcaggcgctctgaccagcggcgtgcacaccttcccg |
| | stant | | Constant | gctgtcctacagtcctcaggactctactccctcagcagcgtggtgaccgtgccctccag |
| | region | | Region | caacttcggcacccagacctacacctgcaacgtagatcacaagcccagcaacaccaagg |
| | | | (IGHG2*06) | tggacaagacagttgagcgcaaatgttgtgtcgagtgcccaccgtgcccagcaccac |
| | | | Nucleotide | ctgtggcaggaccgtcagtcttcctcttccccccaaaacccaaggacaccctcatgatc |
| | | | Sequence | tcccggacccctgaggtcacgtgcgtggtggtggacgtgagccacga |
| | | | | agaccccgaggtccagttcaactggtacgtggacggcgtggagg |
| | | | | tgcataatgccaagacaaagccacgggaggagcagttcaacagcacgttccgtgtggtc |
| | | | | agcgtcctcaccgtcgtgcaccaggactggctgaacggcaaggagta |
| | | | | caagtgcaaggtctccaacaaaggcctcccagcccccatcgagaaaaccatctc |
| | | | | caaaaccaaagggcagccccgagaaccacaggtgtacaccctgcccccatcccgggagg |
| | | | | agatgaccaagaaccaggtcagcctgacctgcctggtcaaaggcttctacccc |
| | | | | agcgacatctccgtggagtgggagagcaatgggcagccggagaacaactacaag |
| | | | | accacacctcccatgctggactccgacggctccttcttcctctacagcaagctcacc |
| | | | | gtggacaagagcaggtggcagcaggggaacgtcttctcatgctccgtgatgcatgagg |
| | | | | ctctgcacaaccactacacacagaagagcctctccctgtctccgggtaaa |
| 257 | | | Human | ASTKGPSVFPLAPCSRSTSESTAALGCLVKDYFPEPVTVSWNSGALTSGVHTFPAVLQSSGLYSLS |
| | | | Heavy | SVVTVPSSNFGTQTYTCNVDHKPSNTKVDKTVERKCCVECPPCPAPPVAGPSVFLFPPKPKDTLMI |
| | | | Chain | SRTPEVTCVVVDVSHEDPEVQFNWYVDGVEVHNAKTKPREEQFNSTFRVVSVLTVVHQDWLNG |
| | | | Constant | KEYKCKVSNKGLPAPIEKTISKTKGQPREPQVYTLPPSREEMTKNQVSLTCLVKGFYPSDISVEWE |
| | | | Region | SNGQPENNYKTTPPMLDSDGSFFLYSKLTVDKSRWQQGNVFSCSVMHEALHNHYTQKSLSLSPG |
| | | | (IGHG2*06) | K |
| | | | Protein | |
| | | | Sequence | |
| 258 | Human | IGHG4 | Human | gcttccaccaagggcccatccgtcttccccctggcgccctgctccaggagcacctccga |
| | IgG4 | *01 | Heavy | gagcacagccgccctgggctgcctggtcaaggactacttccccgaaccggt |
| | con- | or | Chain | gacggtgtcgtggaactcaggcgccctgaccagcggcgtgcacaccttcccg |
| | stant | IGHG4 | Constant | gctgtcctacagtcctcaggactctactccctcagcagcgtggtgaccgtgccctcca |
| | region | *04 | Region | gcagcttgggcacgaagacctacacctgcaacgtagatcacaagcccagcaa |
| | | | (IGHG4*01 | caccaaggtggacaagagagttgagtccaaatatggtcccccatgcccatcat |
| | | | or | gcccagcacctgagttcctggggggaccatcagtcttcctgttccccccaaaacccaa |
| | | | IGHG4*04) | ggacactctcatgatctcccggacccctgaggtcacgtgcgtggtggtggacg |
| | | | Nucleotide | tgagccaggaagaccccgaggtccagttcaactggtacgtggatggcgtgga |
| | | | Sequence | ggtgcataatgccaagacaaagccgcgggaggagcagttcaacagcacgtaccgtgtggt |
| | | | | cagcgtcctcaccgtcctgcaccaggactggctgaacggcaaggagta |
| | | | | caagtgcaaggtctccaacaaaggcctcccgtcctccatcgagaaaaccatc |
| | | | | tccaaagccaaagggcagccccgagagccacaggtgtacaccctgcccccatcccaggag |
| | | | | gagatgaccaagaaccaggtcagcctgacctgcctggtcaaaggcttctaccc |
| | | | | cagcgacatcgccgtggagtgggagagcaatgggcagccggagaacaactacaaga |
| | | | | ccacgcctcccgtgctggactccgacggctccttcttcctctacagcaggctaacc |
| | | | | gtggacaagagcaggtggcaggaggggaatgtcttctcatgctccgtgatg |
| | | | | catgaggctctgcacaaccactacacacagaagagcctctccctgtctctgggtaaa |
| 259 | | | Human | ASTKGPSVFPLAPCSRSTSESTAALGCLVKDYFPEPVTVSWNSGALTSGVHTFPAVLQSSGLYSLS |
| | | | Heavy | SVVTVPSSSLGTKTYTCNVDHKPSNTKVDKRVESKYGPPCPSCPAPEFLGGPSVFLFPPKPKDTLM |
| | | | Chain | ISRTPEVTCVVVDVSQEDPEVQFNWYVDGVEVHNAKTKPREEQFNSTYRVVSVLTVLHQDWLNG |
| | | | Constant | KEYKCKVSNKGLPSSIEKTISKAKGQPREPQVYTLPPSQEEMTKNQVSLTCLVKGFYPSDIAVEWE |
| | | | Region | SNGQPENNYKTTPPVLDSDGSFFLYSRLTVDKSRWQEGNVFSCSVMHEALHNHYTQKSLSLSLG |
| | | | (IGHG4*01) | K |
| | | | Protein | |
| | | | Sequence | |
| | | | (P01861) | |
| 260 | Human | IGHG4 | Human | gcttccaccaagggcccatccgtcttccccctggcgccctgctccaggagcac |
| | IgG4 | *02 | Heavy | ctccgagagcacagccgccctgggctgcctggtcaaggactacttccccgaaccggtga |
| | con- | | Chain | cggtgtcgtggaactcaggcgccctgaccagcggcgtgcacaccttcccg |
| | stant | | Constant | gctgtcctacagtcctcaggactctactccctcagcagcgtggtgaccgtgccctcca |
| | region | | Region | gcagcttgggcacgaagacctacacctgcaacgtagatcacaagcccagcaacac |
| | | | (IGHG4*02) | caaggtggacaagagagttgagtccaaatatggtcccccgtgcccatcatgccca |
| | | | Nucleotide | gcacctgagttcctggggggaccatcagtcttcctgttccccccaaaacccaaggacac |
| | | | Sequence | tctcatgatctcccggacccctgaggtcacgtgcgtggtggtggacgtgagcc |
| | | | | aggaagaccccgaggtccagttcaactggtacgtggatggcgtggaggtgcata |
| | | | | atgccaagacaaagccgcgggaggagcagttcaacagcacgtaccgtgtggtcagcg |
| | | | | tcctcaccgtcgtgcaccaggactggctgaacggcaaggagtacaagtgcaa |
| | | | | ggtctccaacaaaggcctcccgtcctccatcgagaaaaccatctccaaagccaaagg |
| | | | | gcagccccgagagccacaggtgtacaccctgcccccatcccaggaggagatgaccaa |
| | | | | gaaccaggtcagcctgacctgcctggtcaaaggcttctaccccagcgacatcgcc |
| | | | | gtggagtgggagagcaatgggcagccggagaacaactacaagaccacgcctccc |
| | | | | gtgctggactccgacggctccttcttcctctacagcaggctaaccgtggacaagagc |
| | | | | aggtggcaggaggggaatgtcttctcatgctccgtgatgcatgaggctctgcacaac |
| | | | | cactacacgcagaagagcctctccctgtctctgggtaaa |
| 261 | | | Human | ASTKGPSVFPLAPCSRSTSESTAALGCLVKDYFPEPVTVSWNSGALTSGVHTFPAVLQSSGLYSLS |
| | | | Heavy | SVVTVPSSSLGTKTYTCNVDHKPSNTKVDKRVESKYGPPCPSCPAPEFLGGPSVFLFPPKPKDTLM |
| | | | Chain | ISRTPEVTCVVVDVSQEDPEVQFNWYVDGVEVHNAKTKPREEQFNSTYRVVSVLTVVHQDWLN |
| | | | Constant | GKEYKCKVSNKGLPSSIEKTISKAKGQPREPQVYTLPPSQEEMTKNQVSLTCLVKGFYPSDIAVEW |
| | | | Region | ESNGQPENNYKTTPPVLDSDGSFFLYSRLTVDKSRWQEGNVFSCSVMHEALHNHYTQKSLSLSL |
| | | | (IGHG4*02) | GK |
| | | | Protein | |
| | | | Sequence | |
| 262 | Human | IGHG4 | Human | gcttccaccaagggcccatccgtcttccccctggcgccctgctccaggagcacctcc |
| | IgG4 | *03 | Heavy | gagagcacagccgccctgggctgcctggtcaaggactacttccccgaaccgg |
| | con- | | Chain | tgacggtgtcgtggaactcaggcgccctgaccagcggcgtgcacaccttcccg |
| | stant | | Constant | gctgtcctacagtcctcaggactctactccctcagcagcgtggtgaccgtgccctcca |
| | region | | Region | agcagcttgggcacgaagacctaccctgcaacgtagatcacaagcccagcaa |
| | | | (IGHG4*03) | caccaaggtggacaagagagttgagtccaaatatggtcccccatgcccatcat |
| | | | Nucleotide | gcccagcacctgagttcctggggggaccatcagtcttcctgttccccccaaaacccaa |
| | | | Sequence | ggacactctcatgatctcccggacccctgaggtcacgtgcgtggtggtggacg |
| | | | | tgagccaggaagaccccgaggtccagttcaactggtacgtggatggcgtgga |
| | | | | ggtgcataatgccaagacaaagccgcgggaggagcagttcaacagcacgtaccgtgt |
| | | | | ggtcagcgtcctcaccgtcctgcaccaggactggctgaacggcaaggagtac |
| | | | | aagtgcaaggtctccaacaaaggcctcccgtcctccatcgagaaaaccatc |
| | | | | tccaaagccaaagggcagccccgagagccacaggtgtacaccctgcccccatccc |
| | | | | aggaggagatgaccaagaaccaggtcagcctgacctgcctggtcaaaggc |
| | | | | ttctaccccagcgacatcgccgtggagtgggagagcaatgggcagccggaga |
| | | | | acaactacaagaccacgcctcccgtgctggactccgacggctccttcttcctcta |
| | | | | cagcaagctcaccgtggacaagagcaggtggcaggaggggaacgtcttctcatgctccg |
| | | | | tgatgcatgaggctctgcacaaccactacacgcagaagagcctctccctgtctctgggtaaa |
| 263 | | | Human | ASTKGPSVFPLAPCSRSTSESTAALGCLVKDYFPEPVTVSWNSGALTSGVHTFPAVLQSSGLYSLS |
| | | | Heavy | SVVTVPSSSLGTKTYTCNVDHKPSNTKVDKRVESKYGPPCPSCPAPEFLGGPSVFLFPPKPKDTLM |
| | | | Chain | ISRTPEVTCVVVDVSQEDPEVQFNWYVDGVEVHNAKTKPREEQFNSTYRVVSVLTVLHQDWLNG |
| | | | Constant | KEYKCKVSNKGLPSSIEKTISKAKGQPREPQVYTLPPSQEEMTKNQVSLTCLVKGFYPSDIAVEWE |
| | | | Region | SNGQPENNYKTTPPVLDSDGSFFLYSKLTVDKSRWQEGNVFSCSVMHEALHNHYTQKSLSLSLG |
| | | | (IGHG4*03) | K |
| | | | Protein | |
| | | | Sequence | |
| 264 | Human | IGHG4- | Human | gcctccaccaagggcccatccgtcttccccctggcgccctgctccaggag |
| | IgG4- | PE | Heavy | cacctccgagagcacggccgccctgggctgcctggtcaaggactacttcccc |
| | PE | | Chain | gaaccagtgacggtgtcgtggaactcaggcgccctgaccagcggcgtgcacaccttcccg |
| | con- | | Constant | gctgtcctacagtcctcaggactctactccctcagcagcgtggtgaccgtgccctccagc |
| | stant | | Region | agcttgggcacgaagacctacacctgcaacgtagatcacaagcccagcaacaccaagg |
| | region | | (IGHG4-PE) | tggacaagagagttgagtccaaatatggtcccccatgcccaccatgcccagcgcctg |
| | | | Nucleotide | aatttgaggggggaccatcagtcttcctgttccccccaaaacccaaggacactctcatga |
| | | | Sequence | tctcccggacccctgaggtcacgtgcgtggtggtggacgtgagccaggaagac |
| | | | Version A | cccgaggtccagttcaactggtacgtggatggcgtggaggtgcataatgccaagacaaa |
| | | | | gccgcgggaggagcagttcaacagcacgtaccgtgtggtcagcgtcctcaccgtcctg |
| | | | | caccaggactggctgaacggcaaggagtacaagtgcaaggtctccaacaaaggcctccc |
| | | | | gtcatcgatcgagaaaaccatctccaaagccaaagggcagccccgagagccacaggt |
| | | | | gtacaccctgcccccatcccaggaggagatgaccaagaaccaggtcagcctgacctgc |
| | | | | ctggtcaaaggcttctaccccagcgacatcgccgtggagtgggagagcaatgggcagc |
| | | | | cggagaacaactacaagaccacgcctcccgtgctggactccgacggatccttcttcctc |
| | | | | tacagcaggctaaccgtggacaagagcaggtggcaggaggggaatgtcttctcatgct |
| | | | | ccgtgatgcatgaggctctgcacaaccactacacacagaagagcctctcc |
| | | | | ctgtctctgggtaaa |
| 265 | | | Human | gcctccaccaagggacctagcgtgttccctctcgccccctgttccaggtccacaagcga |
| | | | Heavy | gtccaccgctgccctcggctgtctggtgaaagactactttcccgagcccgtga |
| | | | Chain | ccgtctcctggaatagcggagccctgacctccggcgtgcacacatttcccgcc |
| | | | Constant | gtgctgcagagcagcggactgtatagcctgagcagcgtggtgaccgtgcccagctcc |
| | | | Region | agcctcggcaccaaaacctacacctgcaacgtggaccacaagccctccaacacca |
| | | | (IGHG4-PE) | aggtggacaagcgggtggagagcaagtacggccccccttgccctccttgtcctg |
| | | | Nucleotide | cccctgagttcgagggaggaccctccgtgttcctgtttccccccaaacccaaggac |
| | | | Sequence | accctgatgatctcccggacacccgaggtgacctgtgtggtcgtgga |
| | | | Version B | cgtcagccaggaggaccccgaggtgcagttcaactggtatgtggacggcgtgga |
| | | | | ggtgcacaatgccaaaaccaagcccagggaggagcagttcaattccacctacagg |
| | | | | gtggtgagcgtgctgaccgtcctgcatcaggattggctgaacggcaaggag |
| | | | | tacaagtgcaaggtgtccaacaagggactgcccagctccatcgagaagaccat |
| | | | | cagcaaggctaagggccagccgagggagccccaggtgtataccctgcctcctag |
| | | | | ccaggaagagatgaccaagaaccaagtgtccctgacctgcctggtgaaggga |
| | | | | ttctacccctccgacatcgccgtggagtgggagagcaatggccagcccgaga |
| | | | | acaactacaaaacaacccctcccgtgctcgatagcgacggcagcttctttctcta |
| | | | | cagccggctgacagtggacaagagcaggtggcaggagggcaacgtgttctcctg |
| | | | | ttccgtgatgcacgaggccctgcacaatcactacacccagaagagcctctccct |
| | | | | gtccctgggcaag |
| 266 | | | Human | gccagcaccaagggcccttccgtgttccccctggccccttgcagcaggagcacct |
| | | | Heavy | ccgaatccacagctgccctgggctgtctggtgaaggactactttcccgagcccgt |
| | | | Chain | gaccgtgagctggaacagcggcgctctgacatccggcgtccacacctttcctgc |
| | | | Constant | cgtcctgcagtcctccggcctctactccctgtcctccgtggtgaccgtgcctag |
| | | | Region | ctcctccctcggcaccaagacctacacctgtaacgtggaccacaaaccctcc |
| | | | (IGHG4-PE) | aacaccaaggtggacaaacgggtcgagagcaagtacggccctccctgccctccttgtcct |
| | | | Nucleotide | gcccccgagttcgaaggcggacccagcgtgttcctgttccctcctaagcccaaggacacc |
| | | | Sequence | ctcatgatcagccggacacccgaggtgacctgcgtggtggtggatgtgagccaggagga |
| | | | Version C | ccctgaggtccagttcaactggtatgtggatggcgtggaggtgcacaacgccaagacaa |
| | | | | agccccgggaagagcagttcaactccacctacagggtggtcagcgtgctgaccgtgctg |
| | | | | catcaggactggctgaacggcaaggagtacaagtgcaaggtcagcaataagggactgcc |
| | | | | cagcagcatcgagaagaccatctccaaggctaaaggccagccccgggaacctca |
| | | | | ggtgtacaccctgcctcccagccaggaggagatgaccaagaaccaggtgagcctgac |
| | | | | ctgcctggtgaagggattctacccttccgacatcgccgtggagtgggagtccaacggc |
| | | | | cagcccgagaacaattataagaccacccctcccgtcctcgacagcgacggatcc |
| | | | | ttctttctgtactccaggctgaccgtggataagtccaggtggcaggaaggcaacgt |
| | | | | gttcagctgctccgtgatgcacgaggccctgcacaatcactacacccagaagtccct |
| | | | | gagcctgtccctgggaaag |
| 267 | | | Human | ASTKGPSVFPLAPCSRSTSESTAALGCLVKDYFPEPVTVSWNSGALTSGVHTFPAVLQSSGLYSLS |
| | | | Heavy | SVVTVPSSSLGTKTYTCNVDHKPSNTKVDKRVESKYGPPCPPCPAPEFEGGPSVFLFPPKPKDTLM |
| | | | Chain | ISRTPEVTCVVVDVSQEDPEVQFNWYVDGVEVHNAKTKPREEQFNSTYRVVSVLTVLHQDWLNG |
| | | | Constant | KEYKCKVSNKGLPSSIEKTISKAKGQPREPQVYTLPPSQEEMTKNQVSLTCLVKGFYPSDIAVEWE |
| | | | Region | SNGQPENNYKTTPPVLDSDGSFFLYSRLTVDKSRWQEGNVFSCSVMHEALHNHYTQKSLSLSLG |
| | | | (IGHG4-PE) | K |
| | | | Protein | |
| | | | Sequence | |
| | | | (Amino | |
| | | | acid | |
| | | | substi- | |
| | | | tution | |
| | | | shown in | |
| | | | BOLD) | |
| 268 | Inac- | Inact- | Inacti- | gcctccaccaagggcccatccgtcttccccctggcgccctgctccaggagcacctccgaga |
| | ti- | ivated | vated | gcacggccgccctgggctgcctggtcaaggactacttccccgaaccagtgacggtgtcgt |
| | vated | IGHG4 | Human | ggaactcaggcgccctgaccagcggcgtgcacaccttcccggctgtcctacagtcctcagg |
| | Human | | Heavy | actctactccctcagcagcgtggtgaccgtgccctccagcagcttgggcacgaagacctaca |
| | IgG4 | | Chain | cctgcaacgtagatcacaagcccagcaacaccaaggtggacaagagagttgagtccaaatat |
| | con- | | Constant | ggtcccccatgcccaccatgcccagcgcctccagttgcggggggaccatcagtcttcctg |
| | stant | | Region | ttccccccaaaacccaaggacactctcatgatctcccggacccctgaggtcacgtgcgtg |
| | region | | (IGHG4) | gtggtggacgtgagccaggaagaccccgaggtccagttcaactggtacgtggatggcgtgg |
| | | | Nucleotide | aggtgcataatgccaagacaaagccgcgggaggagcagttcaacagcacgtaccgtgtggt |
| | | | Sequence | cagcgtcctcaccgtcctgcaccaggactggctgaacggcaaggagtacaagtgcaagg |
| | | | | tctccaacaaaggcctcccgtcatcgatcgagaaaaccatctccaaagccaaagggcag |
| | | | | ccccgagagccacaggtgtacaccctgcccccatcccaggaggagatgaccaagaacca |
| | | | | ggtcagcctgacctgcctggtcaaaggcttctaccccagcgacatcgccgtggagtggga |
| | | | | gagcaatgggcagccggagaacaactacaagaccacgcctcccgtgctggactcc |
| | | | | gacggatccttcttcctctacagcaggctaaccgtggacaagagcaggtggcagga |
| | | | | ggggaatgtcttctcatgctccgtgatgcatgaggctctgcacaaccactacacac |
| | | | | agaagagcctctccctgtctctgggtaaa |
| 269 | | | Inacti- | ASTKGPSVFPLAPCSRSTSESTAALGCLVKDYFPEPVTVSWNSGALTSGVHTFPAVLQSSGLYSLS |
| | | | vated | SVVTVPSSSLGTKTYTCNVDHKPSNTKVDKRVESKYGPPCPPCPAPPVAGGPSVFLFPPKPKDTL |
| | | | Human | MISRTPEVTCVVVDVSQEDPEVQFNWYVDGVEVHNAKTKPREEQFNSTYRVVSVLTVLHQDWL |
| | | | Heavy | NGKEYKCKVSNKGLPSSIEKTISKAKGQPREPQVYTLPPSQEEMTKNQVSLTCLVKGFYPSDIAVE |
| | | | Chain | WESNGQPENNYKTTPPVLDSDGSFFLYSRLTVDKSRWQEGNVFSCSVMHEALHNHYTQKSLSLS |
| | | | Constant | LGK |
| | | | Region | |
| | | | (IGHG4) | |
| | | | Protein | |
| | | | Sequence | |
| | | | (inacti- | |
| | | | vating | |
| | | | mutations | |
| | | | from human | |
| | | | IgG4 shown | |
| | | | in bold) | |
| 270 | Human | IGKC | Human Cκ | cgtacggtggccgctccctccgtgttcatcttcccaccttccgacgagcagctgaagtccgg |
| | Cκ | *01 | Light | caccgcttctgtcgtgtgcctgctgaacaacttctacccccgcgaggccaaggtgcagt |
| | con- | | Chain | ggaaggtggacaacgccctgcagtccggcaactcccaggaatccgtgaccgagcagga |
| | stant | | Constant | ctccaaggacagcacctactccctgtcctccaccctgaccctgtccaaggccgactacgaga |
| | region | | Region | agcacaaggtgtacgcctgcgaagtgacccaccagggcctgtctagccccgt |
| | | | (IGKC*01) | gaccaagtctttcaaccggggcgagtgt |
| | | | Nucleotide | |
| | | | Sequence | |
| 271 | | | Cκ Light | RTVAAPSVFIFPPSDEQLKSGTASVVCLLNNFYPREAKVQWKVDNALQSGNSQESVTEQDSKDST |
| | | | Chain | YSLSSTLTLSKADYEKHKVYACEVTHQGLSSPVTKSFNRGEC |
| | | | Constant | |
| | | | Region | |
| | | | (IGKC*01) | |
| | | | Amino Acid | |
| | | | Sequence | |
| 272 | Human | IGKC | Cκ Light | cgaactgtggctgcaccatctgtcttcatcttcccgccatctgatgagcagttgaaatctggc |
| | Cκ | *02 | Chain | aactgcctctgttgtgtgcctgtgaataacttctatcccagagaggccaaagtacagtggaag |
| | con- | | Constant | gtggataacgccctccaatcgggtaactcccaggagagtgtcacagagcaggagagcaaggac |
| | stant | | Region | agcacctacagcctcagcagcaccctgacgctgagcaaagcagactacgagaaacacaaagtc |
| | region | | (IGKC*02) | tacgccggcgaagtcacccatcagggcctgagctcgcccgtcacaaagagcttcaacagggga |
| | | | Nucleotide | gagtgt |
| | | | Sequence | |
| 273 | | | Cκ Light | RTVAAPSVFIFPPSDEQLKSGTASVVCLLNNFYPREAKVQWKVDNALQSGNSQESVTEQESKDST |
| | | | Chain | YSLSSTLTLSKADYEKHKVYAGEVTHQGLSSPVTKSFNRGEC |
| | | | Constant | |
| | | | Region | |
| | | | (IGKC*02) | |
| | | | Amino Acid | |
| | | | Sequence | |
| 274 | Human | IGKC | Cκ Light | cgaactgtggctgcaccatctgtcttcatcttcccgccatctgatgagcagttgaaatctggac |
| | Cκ | *03 | Chain | actgcctctgttgtgtgcctgtgaataacttctatcccagagaggccaaagtacagcgg |
| | con- | | Constant | aaggtggataacgccctccaatcgggtaactcccaggagagtgtcacagagcaggagagca |
| | stant | | Region | aggacagcacctacagcctcagcagcaccctgacgctgagcaaagcagactacgagaaa |
| | region | | (IGKC*03) | cacaaagtctacgcctgcgaagtcacccatcagggcctgagctcgcccgtcacaaagagcttc |
| | | | Nucleotide | aacaggggagagtgt |
| | | | Sequence | |
| 275 | | | Cκ Light | RTVAAPSVFIFPPSDEQLKSGTASVVCLLNNFYPREAKVQRKVDNALQSGNSQESVTEQESKDST |
| | | | Chain | YSLSSTLTLSKADYEKHKVYACEVTHQGLSSPVTKSFNRGEC |
| | | | Constant | |
| | | | Region | |
| | | | (IGKC*03) | |
| | | | Amino Acid | |
| | | | Sequence | |
| 276 | Human | IGKC | Cκ Light | cgaactgtggctgcaccatctgtcttcatcttcccgccatctgatgagcagttgaaatctggaa |
| | Cκ | *04 | Chain | ctgcctctgttgtgtgcctgctgaataacttctatcccagagaggccaaagtacagtggaag |
| | con- | | Constant | gtggataacgccctccaatcgggtaactcccaggagagtgtcacagagcaggacagcaagga |
| | stant | | Region | cagcacctacagcctcagcagcaccctgacgctgagcaaagcagactacgagaaa |
| | region | | (IGKC*04) | cacaaactctacgcctgcgaagtcacccatcagggcctgagctcgcccgtcacaaagagc |
| | | | Nucleotide | ttcaacaggggagagtgt |
| | | | Sequence | |
| 277 | | | Cκ Light | RTVAAPSVFIFPPSDEQLKSGTASVVCLLNNFYPREAKVQWKVDNALQSGNSQESVTEQDSKDST |
| | | | Chain | YSLSSTLTLSKADYEKHKLYACEVTHQGLSSPVTKSFNRGEC |
| | | | Constant | |
| | | | Region | |
| | | | (IGKC*04) | |
| | | | Amino Acid | |
| | | | Sequence | |
| 278 | Human | IGKC | Cκ Light | cgaactgtggctgcaccatctgtcttcatcttcccgccatctgatgagcagttgaaatctggaa |
| | Cκ | *05 | Chain | ctgcctctgttgtgtgcctgctgaataacttctatcccagagaggccaaagtacagtggaagg |
| | con- | | Constant | tggataacgccctccaatcgggtaactcccaggagagtgtcacagagcaggacagcaaggacag |
| | stant | | Region | cacctacagcctcagcaacaccctgacgctgagcaaagcagactacgagaaacacaaagtctac |
| | region | | (IGKC*05) | gcctgcgaagtcacccatcagggcctgagctcgcccgtcacaaagagcttcaacaggggagagt |
| | | | Nucleotide | gc |
| | | | Sequence | |
| 279 | | | Cκ Light | RTVAAPSVFIFPPSDEQLKSGTASVVCLLNNFYPREAKVQWKVDNALQSGNSQESVTEQDSKDST |
| | | | Chain | YSLSNTLTLSKADYEKHKVYACEVTHQGLSSPVTKSFNRGEC |
| | | | Constant | |
| | | | Region | |
| | | | (IGKC*05) | |
| | | | Amino Acid | |
| | | | Sequence | |
| 280 | Human | IGLC1 | Cλ Light | cccaaggccaaccccacggtcactctgttcccgccctcctctgaggagctccaagccaacaagg |
| | Cλ | *01 | Chain | ccacactagtgtgtctgatcagtgacttctacccgggagctgtgacagtggcttggaaggcaga |
| | con- | | Constant | tggcagccccgtcaaggcgggagtggagacgaccaaaccctccaaacagagcaacaacaagtac |
| | stant | | Region | gcggccagcagctacctgagcctgacgcccgagcagtggaagtcccacagaagctacagctgcc |
| | region | | (IGLC1*01) | aggtcacgcatgaagggagcaccgtggagaagacagtggcccctacagaatgttca |
| | | | Nucleotide | |
| | | | Sequence | |
| | | | (ENST0000 | |
| | | | 0390321.2) | |
| 281 | | | Cλ Light | PKANPTVTLFPPSSEELQANKATLVCLISDFYPGAVTVAWKADGSPVKAGVETTKPSKQSNNKYA |
| | | | Chain | ASSYLSLTPEQWKSHRSYSCQVTHEGSTVEKTVAPTECS |
| | | | Constant | |
| | | | Region | |
| | | | (IGLC1*01) | |
| | | | Amino Acid | |
| | | | Sequence | |
| | | | (A0A075B6 | |
| | | | K8) | |
| 282 | Human | IGLC1 | Cλ Light | ggtcagcccaaggccaaccccactgtcactctgttcccgccctcctctgaggagctccaagccaa |
| | Cλ | *02 | Chain | caaggccacactagtgtgtctgatcagtgacttctacccgggagctgtgacagtggcctggaag |
| | con- | | Constant | gcagatggcagccccgtcaaggcgggagtggagaccaccaaaccctccaaacagagcaacaaca |
| | stant | | Region | agtacgcggccagcagctacctgagcctgacgcccgagcagtggaagtcccacagaagctac |
| | region | | (IGLC1*02) | agctgccaggtcacgcatgaagggagcaccgtggagaagacagtggcccctacagaatgttca |
| | | | Nucleotide | |
| | | | Sequence | |
| | | | Version A | |
| 283 | | | Cλ Light | ggtcagcccaaggccaaccccactgtcactctgttcccgccctcctctgaggagctccaagccaa |
| | | | Chain | caaggccacactagtgtgtctgatcagtgacttctacccgggagctgtgacagtggcctggaag |
| | | | Constant | gcagatggcagccccgtcaaggcgggagtggagaccaccaaaccctccaaacagagcaacaaca |
| | | | Region | agtacgcggccagcagctacctgagcctgacgcccgagcagtggaagtcccacagaagctaca |
| | | | (IGLC1*02) | gctgccaggtcacgcatgaagggagcaccgtggagaagacagtggcccctacagaatgttca |
| | | | Nucleotide | |
| | | | Sequence | |
| | | | Version B | |
| 284 | | | Cλ Light | GQPKANPTVTLFPPSSEELQANKATLVCLISDFYPGAVTVAWKADGSPVKAGVETTKPSKQSNNK |
| | | | Chain | YAASSYLSLTPEQWKSHRSYSCQVTHEGSTVEKTVAPTECS |
| | | | Constant | |
| | | | Region | |
| | | | (IGLC1*02) | |
| | | | Amino Acid | |
| | | | Sequence | |
| 285 | Human | IGLC2 | Cλ Light | ggccagcctaaggccgctccttctgtgaccctgttccccccatcctccgaggaactgcaggcta |
| | Cλ | *01 | Chain | acaaggccaccctcgtgtgcctgatcagcgacttctaccctggcgccgtgaccgtggcctggaa |
| | con- | | Constant | ggctgatagctctcctgtgaaggccggcgtggaaaccaccaccccttccaagcagtccaacaac |
| | stant | | Region | aaatacgccgcctcctcctacctgtccctgacccctgagcagtggaagtcccaccggtcctac |
| | region | | (IGLC2*01) | agctgccaagtgacccacgagggctccaccgtggaaaagaccgtggctcctaccgagtgctcc |
| | | | Nucleotide | |
| | | | Sequence | |
| | | | Version A | |
| 286 | | | Cλ Light | ggccagcctaaagctgcccccagcgtcaccctgtttcctccctccagcgaggagctccaggcca |
| | | | Chain | acaaggccaccctcgtgtgcctgatctccgacttctatcccggcgctgtgaccgtggcttggaa |
| | | | Constant | agccgactccagccctgtcaaagccggcgtggagaccaccacaccctccaagcagtccaacaac |
| | | | Region | aagtacgccgcctccagctatctctccctgacccctgagcagtggaagtcccaccggtcctac |
| | | | (IGLC2*01) | tcctgtcaggtgacccacgagggctccaccgtggaaaagaccgtcgcccccaccgagtgctcc |
| | | | Nucleotide | |
| | | | Sequence | |
| | | | Version B | |
| 287 | | | Cλ Light | GQPKAAPSVTLFPPSSEELQANKATLVCLISDFYPGAVTVAWKADSSPVKAGVETTTPSKQSNNK |
| | | | Chain | YAASSYLSLTPEQWKSHRSYSCQVTHEGSTVEKTVAPTECS |
| | | | Constant | |
| | | | Region | |
| | | | (IGLC1*02) | |
| | | | Amino Acid | |
| | | | Sequence | |
| 288 | Human | IGLC2 | Cλ Light | ggtcagcccaaggctgccccctcggtcactctgttcccgccctcctctgaggagcttcaagcca |
| | Cλ | *02 | Chain | acaaggccacactggtgtgtctcataagtgacttctacccgggagccgtgacagtggcctggaa |
| | con- | or | Constant | ggcagatagcagccccgtcaaggcgggagtggagaccaccacaccctccaaacaaagcaacaac |
| | stant | IGLC2 | Region | aagtacgcggccagcagctatctgagcctgacgcctgagcagtggaagtcccacagaagctaca |
| | region | *03 | (IGLC2*02 | gctgccaggtcacgcatgaagggagcaccgtggagaagacagtggcccctacagaatgttca |
| | | | or | |
| | | | IGLC2*03) | |
| | | | Nucleotide | |
| | | | Sequence | |
| 289 | | | Cλ Light | GQPKAAPSVTLFPPSSEELQANKATLVCLISDFYPGAVTVAWKADSSPVKAGVETTTPSKQSNNK |
| | | | Chain | YAASSYLSLTPEQWKSHRSYSCQVTHEGSTVEKTVAPTECS |
| | | | Constant | |
| | | | Region | |
| | | | (IGLC2*02) | |
| | | | Amino Acid | |
| | | | Sequence | |
| 290 | Human | IGLC3 | Cλ Light | cccaaggctgccccctcggtcactctgttcccaccctcctctgaggagcttcaagccaacaag |
| | Cλ | *01 | Chain | gccacactggtgtgtctcataagtgacttctacccgggagccgtgacagttgcctggaaggca |
| | con- | | Constant | gatagcagccccgtcaaggcgggggtggagaccaccacaccctccaaacaaagcaacaacaag |
| | stant | | Region | tacgcggccagcagctacctgagcctgacgcctgagcagtggaagtcccacaaaagctacagc |
| | region | | (IGLC3*01) | tgccaggtcacgcatgaagggagcaccgtggagaagacagttgcccctacggaatgttca |
| | | | Nucleotide | |
| | | | Sequence | |
| 291 | | | Cλ Light | PKAAPSVTLFPPSSEELQANKATLVCLISDFYPGAVTVAWKADSSPVKAGVETTTPSKQSNNKYAA |
| | | | Chain | SSYLSLTPEQWKSHKSYSCQVTHEGSTVEKTVAPTECS |
| | | | Constant | |
| | | | Region | |
| | | | (IGLC3*01) | |
| | | | Amino Acid | |
| | | | Sequence | |
| 292 | Human | IGLC3 | Cλ Light | ggtcagcccaaggctgccccctcggtcactctgttcccaccctcctctgaggagcttcaagcc |
| | Cλ | *02 | Chain | aacaaggccacactggtgtgtctcataagtgacttctacccggggccagtgacagttgcctgg |
| | con- | | Constant | aaggcagatagcagccccgtcaaggcgggggtggagaccaccacaccctccaaacaaagcaac |
| | stant | | Region | aacaagtacgcggccagcagctacctgagcctgacgcctgagcagtggaagtcccacaaaagc |
| | region | | (IGLC3*02) | tacagctgccaggtcacgcatgaagggagcaccgtggagaagacagtggcccctacggaatgt |
| | | | Nucleotide | tca |
| | | | Sequence | |
| 293 | | | Cλ Light | GQPKAAPSVTLFPPSSEELQANKATLVCLISDFYPGPVTVAWKADSSPVKAGVETTTPSKQSNNKY |
| | | | Chain | AASSYLSLTPEQWKSHKSYSCQVTHEGSTVEKTVAPTECS |
| | | | Constant | |
| | | | Region | |
| | | | (IGLC1*02) | |
| | | | Amino Acid | |
| | | | Sequence | |
| 294 | Human | IGLC3 | Cλ Light | ggtcagcccaaggctgccccctcggtcactctgttcccaccctcctctgaggagcttcaagcc |
| | Cλ | *03 | Chain | aacaaggccacactggtgtgtctcataagtgacttctacccgggagccgtgacagtggcctgg |
| | con- | | Constant | aaggcagatagcagccccgtcaaggcgggagtggagaccaccacaccctccaaacaaagcaac |
| | stant | | Region | aacaagtacgcggccagcagctacctgagcctgacgcctgagcagtggaagtcccacaaaagc |
| | region | | (IGLC3*03) | tacagctgccaggtcacgcatgaagggagcaccgtggagaagacagtggcccctacagaatgt |
| | | | Nucleotide | tca |
| | | | Sequence | |
| 295 | | | Cλ Light | GQPKAAPSVTLFPPSSEELQANKATLVCLISDFYPGAVTVAWKADSSPVKAGVETTTPSKQSNNK |
| | | | Chain | YAASSYLSLTPEQWKSHKSYSCQVTHEGSTVEKTVAPTECS |
| | | | Constant | |
| | | | Region | |
| | | | (IGLC3*03) | |
| | | | Amino Acid | |
| | | | Sequence | |
| 296 | Human | IGLC3 | Cλ Light | ggtcagcccaaggctgccccctcggtcactctgttcccgccctcctctgaggagcttcaagcc |
| | Cλ | *04 | Chain | aacaaggccacactggtgtgtctcataagtgacttctacccgggagccgtgacagtggcctgg |
| | con- | | Constant | aaggcagatagcagccccgtcaaggcgggagtggagaccaccacaccctccaaacaaagcaac |
| | stant | | Region | aacaagtacgcggccagcagctacctgagcctgacgcctgagcagtggaagtcccacagaagc |
| | region | | (IGLC3*04) | tacagctgccaggtcacgcatgaagggagcaccgtggagaagacagtggcccctacagaatgt |
| | | | Nucleotide | tca |
| | | | Sequence | |
| 297 | | | Cλ Light | GQPKAAPSVTLFPPSSEELQANKATLVCLISDFYPGAVTVAWKADSSPVKAGVETTTPSKQSNNK |
| | | | Chain | YAASSYLSLTPEQWKSHRSYSCQVTHEGSTVEKTVAPTECS |
| | | | Constant | |
| | | | Region | |
| | | | (IGLC3*04) | |
| | | | Amino Acid | |
| | | | Sequence | |
| 298 | Human | IGLC6 | Cλ Light | ggtcagcccaaggctgccccatcggtcactctgttcccgccctcctctgaggagcttcaagcc |
| | Cλ | *01 | Chain | aacaaggccacactggtgtgcctgatcagtgacttctacccgggagctgtgaaagtggcctgg |
| | con- | | Constant | aaggcagatggcagccccgtcaacacgggagtggagaccaccacaccctccaaacagagcaac |
| | stant | | Region | aacaagtacgcggccagcagctacctgagcctgacgcctgagcagtggaagtcccacagaagc |
| | region | | (IGLC6*01) | tacagctgccaggtcacgcatgaagggagcaccgtggagaagacagtggcccctgcagaatgt |
| | | | Nucleotide | tca |
| | | | Sequence | |
| 299 | | | Cλ Light | GQPKAAPSVTLFPPSSEELQANKATLVCLISDFYPGAVKVAWKADGSPVNTGVETTTPSKQSNNK |
| | | | Chain | YAASSYLSLTPEQWKSHRSYSCQVTHEGSTVEKTVAPAECS |
| | | | Constant | |
| | | | Region | |
| | | | (IGLC6*01) | |
| | | | Amino Acid | |
| | | | Sequence | |
| 300 | Human | IGLC7 | Cλ Light | ggtcagcccaaggctgccccatcggtcactctgttcccaccctcctctgaggagcttcaagcc |
| | Cλ | *01 | Chain | aacaaggccacactggtgtgtctcgtaagtgacttctacccgggagccgtgacagtggcctgg |
| | con- | or | Constant | aaggcagatggcagccccgtcaaggtgggagtggagaccaccaaaccctccaaacaaagcaac |
| | stant | IGLC7 | Region | aacaagtatgcggccagcagctacctgagcctgacgcccgagcagtggaagtcccacagaagc |
| | region | *02 | (IGLC7*01 | tacagctgccgggtcacgcatgaagggagcaccgtggagaagacagtggcccctgcagaatgc |
| | | | or | tct |
| | | | IGLC7*02) | |
| | | | Nucleotide | |
| | | | Sequence | |
| 301 | | | Cλ Light | GQPKAAPSVTLFPPSSEELQANKATLVCLVSDFYPGAVTVAWKADGSPVKVGVETTKPSKQSNNK |
| | | | Chain | YAASSYLSLTPEQWKSHRSYSCRVTHEGSTVEKTVAPAECS |
| | | | Constant | |
| | | | Region | |
| | | | (IGLC7*01) | |
| | | | Amino Acid | |
| | | | Sequence | |
| 302 | Human | IGLC7 | Cλ Light | ggtcagcccaaggctgccccctcggtcactctgttcccaccctcctctgaggagcttcaagcc |
| | Cλ | *03 | Chain | aacaaggccacactggtgtgtctcgtaagtgacttcaacccgggagccgtgacagtggcctgg |
| | con- | | Constant | aaggcagatggcagccccgtcaaggtgggagtggagaccaccaaaccctccaaacaaagcaac |
| | stant | | Region | aacaagtatgcggccagcagctacctgagcctgacgcccgagcagtggaagtcccacagaagc |
| | region | | (IGLC7*03) | tacagctgccgggtcacgcatgaagggagcaccgtggagaagacagtggcccctgcagaatgc |
| | | | Nucleotide | tct |
| | | | Sequence | |
| 303 | | | Cλ Light | GQPKAAPSVTLFPPSSEELQANKATLVCLVSDFNPGAVTVAWKADGSPVKVGVETTKPSKQSNNK |
| | | | Chain | YAASSYLSLTPEQWKSHRSYSCRVTHEGSTVEKTVAPAECS |
| | | | Constant | |
| | | | Region | |
| | | | (IGLC7*03) | |
| | | | Amino Acid | |
| | | | Sequence | |
| 304 | Human | IGHG4- | Human | gcttctaccaagggacccagcgtgttccctctggctccttgctccagatccacctccgagtct |
| | IgG4- | PE | Heavy | acagctgctctgggctgcctggtcaaggactactttcctgagcctgtgaccgtgtcttggaac |
| | PE | | Chain | tctggcgctctgacatctggcgtgcacacattccctgctgtgctgcagtcctccggcctgtac |
| | con- | | Constant | tctctgtcctctgtcgtgaccgtgccttcctctagcctgggcaccaagacctacacctgtaat |
| | stant | | Region | gtggaccacaagccttccaacaccaaggtggacaagcgcgtggaatctaagtacggccctcct |
| | region | | (IGHG4-PE) | tgtcctccatgtcctgctccagagtttgaaggcggcccttccgtgtttctgttccctccaaag |
| | | | Nucleotide | cctaaggacaccctgatgatctctcggacccctgaagtgacctgcgtggtggtggatgtgtcc |
| | | | Sequence | caagaggatcccgaggtgcagttcaattggtacgtggacggcgtggaagtgcacaacgccaag |
| | | | (lysine | accaagcctagagaggaacagttcaactccacctacagagtggtgtccgtgctgaccgtgctg |
| | | | clipped) | caccaggattggctgaacggcaaagagtacaagtgcaaggtgtccaacaagggcctgcctagc |
| | | | | tccatcgaaaagaccatctccaaggccaagggccagcctcgagaaccccaggtttacaccctg |
| | | | | cctccaagccaagaggaaatgaccaagaaccaggtgtccctgacctgcctcgtgaagggattc |
| | | | | tacccctccgatatcgccgtggaatgggagtctaatggccagccagagaacaactacaagaca |
| | | | | acccctcctgtgctggactccgacggctccttctttctgtattcccgcctgaccgtggacaag |
| | | | | tccagatggcaagagggcaacgtgttctcctgcagcgtgatgcacgaggccctg |
| | | | | cacaatcactacacccagaagtccctgtctctgtccctgggc |
| 305 | Human | IGHG4- | Human | ASTKGPSVFPLAPCSRSTSESTAALGCLVKDYFPEPVTVSWNSGALTSGVHTFPAVLQSSGLYSLS |
| | IgG4- | PE | Heavy | SVVTVPSSSLGTKTYTCNVDHKPSNTKVDKRVESKYGPPCPPCPAPEFEGGPSVFLFPPKPKDTLM |
| | PE | | Chain | ISRTPEVTCVVVDVSQEDPEVQFNWYVDGVEVHNAKTKPREEQFNSTYRVVSVLTVLHQDWLNG |
| | con- | | Constant | KEYKCKVSNKGLPSSIEKTISKAKGQPREPQVYTLPPSQEEMTKNQVSLTCLVKGFYPSDIAVEWE |
| | stant | | Region | SNGQPENNYKTTPPVLDSDGSFFLYSRLTVDKSRWQEGNVFSCSVMHEALHNHYTQKSLSLSLG |
| | region | | (IGHG4-PE) | |
| | | | Amino Acid | |
| | | | Sequence | |
| | | | (lysine | |
| | | | clipped) | |
| 306 | Human | IGHG1 | Human | gcttctaccaagggacccagcgttttccctctggctccatcctccaagtctacctctggcgga |
| | IgG1 | (FES | Heavy | acagctgctctgggctgcctggtcaaggactactttcctgagccagtgaccgtgtcttggaac |
| | con- | mu- | Chain | tctggcgctctgacatctggcgtgcacacctttccagctgtgctgcagtcctccggcctgtac |
| | stant | tant) | Constant | tctctgtcctctgtcgtgaccgtgccttccagctctctgggaacccagacctacatctgcaat |
| | region | | Region | gtgaaccacaagccttccaacaccaaggtggacaagaaggtggaacccaagtcctgcgacaag |
| | (FES | | (IGHG1 | acccacacctgtcctccatgtcctgctccagagtttgaaggcggcccttccgtgttcctgttt |
| | mu- | | (FES | cctccaaagcctaaggacaccctgatgatctctcggacccctgaagtgacctgcgtggtggtg |
| | tant) | | mutant)) | gatgtgtctcacgaggatcccgaagtgaagttcaattggtacgtggacggcgtggaagtgcac |
| | | | Nucleotide | aacgccaagaccaagcctagagaggaacagtacaactccacctacagagtggtgtccgtgctg |
| | | | Sequence | accgtgctgcaccaggattggctgaacggcaaagagtacaagtgcaaggtgtccaacaaggcc |
| | | | | ctgcctgcctccatcgaaaagaccatctccaaggccaagggccagcctagggaaccccaggtt |
| | | | | tacaccttgccaccttctcgggacgagctgaccaagaaccaggtgtccctgacctgtctggtt |
| | | | | aagggcttctacccctccgatatcgccgtggaatgggagtctaatggccagcctgagaacaac |
| | | | | tacaagacaacccctcctgtgctggactccgacggctcattcttcctgtactccaagctgaca |
| | | | | gtggacaagtccagatggcagcagggcaacgtgttctcctgctctgtgatgcacgaggccctg |
| | | | | cacaaccactacacccagaagtccctgtctctgagccccggcaag |
| 307 | Human | IGHG1 | Human | ASTKGPSVFPLAPSSKSTSGGTAALGCLVKDYFPEPVTVSWNSGALTSGVHTFPAVLQSSGLYSLS |
| | IgG1 | (FES | Heavy | SVVTVPSSSLGTQTYICNVNHKPSNTKVDKKVEPKSCDKTHTCPPCPAPEFEGGPSVFLFPPKPKD |
| | con- | mu- | Chain | TLMISRTPEVTCVVVDVSHEDPEVKFNWYVDGVEVHNAKTKPREEQYNSTYRVVSVLTVLHQDW |
| | stant | tant) | Constant | LNGKEYKCKVSNKALPASIEKTISKAKGQPREPQVYTLPPSRDELTKNQVSLTCLVKGFYPSDIAVE |
| | region | | Region | WESNGQPENNYKTTPPVLDSDGSFFLYSKLTVDKSRWQQGNVFSCSVMHEALHNHYTQKSLSLS |
| | (FES | | (IGHG1 | PGK |
| | mu- | | (FES | |
| | tant) | | mutant)) | |
| | | | (amino | |
| | | | acid | |
| | | | changes | |
| | | | shown in | |
| | | | bold) | |
| | | | amino acid | |
| | | | sequence | |

| | | | |
|---|---|---|---|
| 308 | IGHV3- | IGHV3- | atggagtttgggctgagctgggttttcctcgttgctcttttaagaggtgtccagtgtcaggtg |
| | 30*18 | 30*18 gene | cagctggtggagtctgggggaggcgtggtccagcctgggaggtccctgagactctcctgtgca |
| | | segment | gcctctggattcaccttcagtagctatggcatgcactgggtccgccaggctccaggcaagggg |
| | | nucleotide | ctggagtgggtggcagttatatcatatgatggaagtaataaatactatgcagactccgtgaag |
| | | sequence | ggccgattcaccatctccagagacaattccaagaacacgctgtatctgcaaatgaacagcctg |
| | | | agagctgaggacacggctgtgtattactgtgcgaaaga |
| 309 | IGHV3- | IGHV3- | atggagtttgggctgagctgggttttccttgttgctattttaaaaggtgtccagtgtgaggtg |
| | 73*02 | 73*02 gene | cagctggtggagtccgggggaggcttggtccagcctggggggtccctgaaactctcctgtgca |
| | | segment | gcctctgggttcaccttcagtggctctgctatgcactgggtccgccaggcttccgggaaaggg |
| | | nucleotide | ctggagtgggttggccgtattagaagcaaagctaacagttacgcgacagcatatgctgcgtcg |
| | | sequence | gtgaaaggcaggttcaccatctccagagatgattcaaagaacacggcgtatctgcaaatgaac |
| | | | agcctgaaaaccgaggacacggccgtgtattactgtactagaca |
| 310 | IGHV4- | IGHV4- | atgaaacacctgtggttcttcctcctgctggtggcagctcccagatgggtcctgtcccaggtg |
| | 31*03 | 31*03 gene | cagctgcaggagtcgggcccaggactggtgaagccttcacagaccctgtccctcacctgcact |
| | | segment | gtctctggtggctccatcagcagtggtggttactactggagctggatccgccagcacccaggg |
| | | nucleotide | aagggcctggagtggattgggtacatctattacagtgggagcacctactacaacccgtccctc |
| | | sequence | aagagtcgagttaccatatcagtagacacgtctaagaaccagttctccctgaagctgagctct |
| | | | gtgactgccgcggacacggccgtgtattactgtgcgagaga |
| 311 | IGHV3- | IGHV3- | atggagtttgggctgagctgggttttcctcgttgctcttttaagaggtgtccagtgtcaggtg |
| | 33*01 | 33*01 gene | cagctggtggagtctgggggaggcgtggtccagcctgggaggtccctgagactctcctgtgca |
| | | segment | gcgtctggattcaccttcagtagctatggcatgcactgggtccgccaggctccaggcaagggg |
| | | nucleotide | ctggagtgggtggcagttatatggtatgatggaagtaataaatactatgcagactccgtgaag |
| | | sequence | ggccgattcaccatctccagagacaattccaagaacacgctgtatctgcaaatgaacagcctg |
| | | | agagccgaggacacggctgtgtattactgtgcgagaga |
| 312 | IGHV3- | IGHV3- | atggagtttgggctgagctggctttttcttgtggctattttaaaaggtgtccagtgtgaggtg |
| | 23*04 | 23*04 gene | cagctggtggagtctgggggaggcttggtacagcctggggggtccctgagactctcctgtgca |
| | | segment | gcctctggattcacctttagcagctatgccatgagctgggtccgccaggctccagggaagggg |
| | | nucleotide | ctggagtgggtctcagctattagtggtagtggtggtagcacatactacgcagactccgtgaag |
| | | sequence | ggccggttcaccatctccagagacaattccaagaacacgctgtatctgcaaatgaacagcctg |
| | | | agagccgaggacacggccgtatattactgtgcgaaaga |
| 313 | IGHD3- | IGHD3- | gtattatgattacgtttgggggagttatcgttatacc |
| | 16*02 | 16*02 gene | |
| | | segment | |
| | | nucleotide | |
| | | sequence | |
| 314 | IGHD3- | IGHD3- | gtattacgatattttgactggttattataac |
| | 9*01 | 9*01 gene | |
| | | segment | |
| | | nucleotide | |
| | | sequence | |
| 315 | IGHD3- | IGHD3- | gtattactatggttcggggagttattataac |
| | 10*01 | 10*01 gene | |
| | | segment | |
| | | nucleotide | |
| | | sequence | |
| 316 | IGHD1- | IGHD1- | ggtataactggaacgac |
| | 20*01 | 20*01 gene | |
| | | segment | |
| | | nucleotide | |
| | | sequence | |
| 317 | IGHD1- | IGHD1- | ggtacaactggaacgac |
| | 1*01 | 1*01 gene | |
| | | segment | |
| | | nucleotide | |
| | | sequence | |
| 318 | IGHJ6 | IGHJ6*02 | attactactactactacggtatggacgtctggggccaagggaccacggtcaccgtctcctcag |
| | *02 | gene | gt |
| | | segment | |
| | | nucleotide | |
| | | sequence | |
| 319 | IGHJ3 | IGHJ3*02 | tgatgcttttgatatctggggccaagggacaatggtcaccgtctcttcaggt |
| | *02 | gene | |
| | | segment | |
| | | nucleotide | |
| | | sequence | |
| 320 | IGKV1- | IGKV1- | atggacatgagggtccccgctcagctcctggggctcctgctgctctggttcccaggtgccagg |
| | 17*01 | 17*01 gene | tgtgacatccagatgacccagtctccatcctccctgtctgcatctgtaggagacagagtcacc |
| | | segment | atcacttgccgggcaagtcagggcattagaaatgatttaggctggtatcagcagaaaccaggg |
| | | nucleotide | aaagcccctaagcgcctgatctatgctgcatccagtttgcaaagtggggtcccatcaaggttc |
| | | sequence | agcggcagtggatctgggacagaattcactctcacaatcagcagcctgcagcctgaagatttt |
| | | | gcaacttattactgtctacagcataatagttaccct |
| 321 | IGLV10- | IGLV10- | atgccctgggctctgctcctcctgaccctcctcactcactctgcagtgtcagtggtccaggca |
| | 54*02 | 54*02 gene | gggctgactcagccaccctcggtgtccaagggcttgagacagaccgccacactcacctgcact |
| | | segment | gggaacagcaacattgttggcaaccaaggagcagcttggctgcagcagcaccagggccaccct |
| | | nucleotide | cccaaactcctatcctacaggaataacaaccggccctcagggatctcagagagattctctgca |
| | | sequence | tccaggtcaggaaacacagcctccctgaccattactggactccagcctgaggacgaggctgac |
| | | | tattactgctcagcattggacagcagcctcagtgctca |
| 322 | IGKV2- | IGKV2- | atgaggctccctgctcagctcctggggctgctaatgctctgggtctctggatccagtggggat |
| | 28*01 | 28*01 gene | attgtgatgactcagtctccactctccctgcccgtcacccctggagagccggcctccatctcc |
| | | segment | tgcaggtctagtcagagcctcctgcatagtaatggatacaactatttggattggtacctgcag |
| | | nucleotide | aagccagggcagtctccacagctcctgatctatttgggttctaatcgggcctccggggtccct |
| | | sequence | gacaggttcagtggcagtggatcaggcacagattttacactgaaaatcagcagagtggaggct |
| | | | gaggatgttggggtttattactgcatgcaagctctacaaactcctcc |
| 323 | IGKJ3 | IGKJ3*01 | attcactttcggccctgggaccaaagtggatatcaaac |
| | *01 | gene | |
| | | segment | |
| | | nucleotide | |
| | | sequence | |
| 324 | IGLJ3 | IGLJ3*02 | ttgggtgttcggcggagggaccaagctgaccgtcctag |
| | *02 | gene | |
| | | segment | |
| | | nucleotide | |
| | | sequence | |
| 325 | IGKJ1 | IGKJ1*01 | gtggacgttcggccaagggaccaaggtggaaatcaaac |
| | *01 | gene | |
| | | segment | |
| | | nucleotide | |
| | | sequence | |
| 326 | IGKJ4 | IGKJ4*01 | gctcactttcggcggagggaccaaggtggagatcaaac |
| | *01 | gene | |
| | | segment | |
| | | nucleotide | |
| | | sequence | |
| 327 | Human | Amino Acid | MNAKVVVVLVLVLTALCLSDGKPVSLSYRCPCRFFESHVARANVKHLKILNTPNCALQIVAR |
| | CXCL12 | Sequence | LKNNNRQVCIDPKLKWIQEYLEKALNK |
| | Alpha | for | |
| | | CXCL12- | |
| | | Alpha. | |
| | | (Uniprot | |
| | | ID: | |
| | | P48061-2) | |

### 8. Examples

The following examples describe the generation, characterisation and performance of anti-CXCR4 antibodies. Antibodies were generated using the Kymouse™ (AKA IntelliSelect™ Transgenic Mouse), a transgenic mouse platform capable of generating antibodies with human variable domains, generated from human V (D) and J gene segments, and mouse constant domains. The Kymouse™ (AKA IntelliSelect™ Transgenic Mouse) system is described in Lee et al., 2014, Complete humanization of the mouse immunoglobulin loci enables efficient therapeutic antibody discovery, Nature Biotechnology, 32(4):356-363., WO2011/004192, WO2011/158009 and WO2013/061098. This project employed the Kymouse™ (AKA IntelliSelec™ Transgenic Mouse) HK and HL strains. In the HK strain, the immunoglobulin heavy chain locus and light chain kappa locus are humanised, and in the HL strain the immunoglobulin heavy chain locus and light chain lambda locus are humanised.

Kymice™ (AKA IntelliSelec™ Transgenic Mice) were immunised with various types of CXCR4 over-expressing cell lines using conventional 12-week cell expressed antigen immunisation schedules that consisted of multiple boosts throughout each immunisation (Table 1), and antibodies which bound to human CXCR4 were identified. The antibodies were subjected to a primary and secondary screen, as detailed further below to identify a lead panel.

Unless otherwise stated, all proteins or peptides (e.g. CXCR4, CXCR7 and CXCL12) are human.

### Example 1

### (A) Preparation of MEF, DC2.4, CHO-S and HEK-293 Cells Expressing CXCR4 or StaR® CXCR4 or CXCR7 for Immunisation and Screening

Stably transfected cell lines were generated. Full length DNA sequence encoding codon optimised (for mammalian expression) CXCR4 (SEQ ID NO: 1) (histidine-tagged human CXCR4), cynomolgus (cyno) CXCR4 (SEQ ID NO: 7), CXCR7 (SEQ ID NO: 4) and StaR® CXCR4 was ordered as synthetic DNA oligonucleotides and cloned into an expression vector under the control of the CMV or EF1α promoter flanked by 3′ and 5′ piggyBac specific terminal repeat sequences, which facilitated stable integration into the cell genome (see: “A hyperactive piggyBac transposase for mammalian applications”; Yusa K., et al., Proc. Natl. Acad. Sci. USA., 108(4): 1531-6, 2011 Jan. 25). For generation of CXCR4 expressing cell lines, human embryonic kidney-293 (HEK-293), Chinese hamster ovary-S(CHO-S) cells, mouse dendritic cells (DC2.4) and mouse embryonic fibroblast (MEF) cells were transfected using the FreeStyle Max transfection reagent (Invitrogen) according to manufacturer instructions. To generate cynomolgus CXCR4, CXCR7 and StaR® CXCR4 cell lines, CMV promoter driven cyno CXCR4, CXCR7 and StaR® CXCR4 expression vectors were co-transfected with piggyBac transposase into CHO-S cells to generate cells singly expressing cyno CXCR4, CXCR7 and StaR® CXCR4 antigens respectively. Expression of CXCR4 or StaR® protein was assessed by flow cytometry using anti-CXCR4-Phycoerythrin (PE)-conjugated antibody (FAB173P, R&D Systems). Expression of cyno CXCR4 was assessed by flow cytometry using anti-CXCR4-PE-conjugated antibody 12G5 (R&D Systems). Expression of CXCR7 was assessed by flow cytometry using anti-CXCR7-PE fluorophore conjugated antibody 8F11-M16 (Biolegend). Following assessment of antigen expression, stable CXCR4 expressing HEK-293 and CHO-S cells and CXCR4 StaR® CHO-S cells were FACS sorted for high CXCR4 expression. Stably expressing CXCR4 MEF cells were made into clonal cell lines using serial dilution method and a single clone was scaled up with high CXCR4 expression.

### Example 2—Immunisation

Kymouse™ (AKA IntelliSelec™ Transgenic Mouse) HK and HL strains were immunised according to the regimens shown in Table 1 with CXCR4 expressing cells (HEK-293, DC2.4 or CHO) or StaR® DNA or StaR® protein (used either alone or in combination for immunisation). Table 1 details strain of Kymouse™ and immunogen used at each immunisation stage (prime and boosts).

TABLE 1
Summary of immunisation performed using Kymouse ™ (AKA
IntelliSelect ™ Transgenic Mouse)
HL or HK strains.

| | | | | | | Final |
|---|---|---|---|---|---|---|
| Regime | Mouse | Prime | Boost 1 | Boost 2 | Boost 3 | Boost |
| KM157-B1 | HK, | hCXCR4 | hCXCR4 | hCXCR4 | hCXCR4 | hCXCR4 |
| | HL | HEK-293 | HEK-293 | HEK-293 | HEK-293 | HEK-293 |
| | | cells | cells | cells | cells | cells |
| KM161-B1 | HK | hCXCR4 | hCXCR4 | hCXCR4 | — | hCXCR4 |
| | | DC2.4 | DC2.4 | DC2.4 | | DC2.4 |
| | | cells | cells | cells | | cells |
| KM173-B1 | HL | hCXCR4 | hCXCR4 | hCXCR4 | — | hCXCR4 |
| | | StaR ® | StaR ® | StaR ® | | StaR ® |
| | | DNA | protein | protein | | protein |

Priming of KM173-B1 with CXCR4 StaR® DNA was achieved using hydrodynamic tail vein injection. Sigma adjuvant system was used for KM157-B1 throughout and KM173-B1 StaR® protein boosts. Sigma adjuvant was only used for KM161-B1 prime. Immunisations and rest intervals were usually between 2 and 3 weeks. Sera from serial blood samples were analysed for the presence of specific antibodies by flow cytometry and the titre data was used (where possible) to select mice to be used for B-cell sorting.

### Example 3—Generation of B-Cell Sorting Antigen and Antibody Retrieval and Expression

(A) CXCR4 StaR® Generation, Purification and Nanodisc Incorporation for B-cell Sorting The generation, expression, purification and nanodisc incorporation of CXCR4 stabilised receptor proteins (StaR®), fused to green fluorescent protein (GFP) to generate fluorescent sorting reagents for labelling and isolation of B-cells expressing CXCR4-reactive B-cell receptors was conducted. A thermostabilised CXCR4 variant was generated by site-directed mutagenesis (essentially as described in WO2009/081136), then cloned into insect cell expression vectors to generate recombinant proteins fused to green fluorescent protein (GFP; StaR®). Fluorescent StaR® proteins were then expressed in insect cells, using the baculovirus expression system, and purified in detergent using affinity and size-exclusion chromatography. Detergent-purified receptors were then incorporated into lipid-protein nanodiscs (see “Preparation Of Nanodisc Reconstitution Mixtures”;www.sigmaaldrich.com/technical-documents/articles/materialsscience/nanomaterials/nanodisc-protocols.html), and detergent was removed, to generate receptor reagents compatible for use with live cells.

### (B) Sorting of B-Cells Expressing Anti-CXCR4 Antibodies

B-cells expressing anti-CXCR4 antibodies were recovered from immunised mice, using techniques substantially as described in Example 1 of WO2015/040401. Immunised mice were selected from each immunisation campaign based on titre response to CXCR4. In brief, spleen and lymph node cells isolated from mice within the immunisation regimes were stained with a mix of fluorophore-conjugated antibodies and markers (CD19-PB, IgM-APC-Cy7, IgD-APC-Cy7, 7-AAD), and fluorescently-tagged StaR® CXCR4 nanodisc-GFP, to allow identification of cells of interest for fluorescence activated cell sorting (FACS). Single cells were selected based on their forward and side scatter properties and 7-AAD⁺ dead cells were excluded. CD19⁺ B-cells were enriched for the class switched population by selecting IgM⁻ IgD⁻ cells and were further selected for binding to StaR® CXCR4-nanodisc-GFP. IgM⁻ IgD⁻ CXCR4⁺ cells were single cell sorted into lysis buffer and snap frozen on dry ice to preserve RNA content.

V-region sequences from the single-cell sorted B-cells were recovered using RT-PCR and two further rounds of PCR, then bridged to mouse IgG1 constant region and expressed in HEK-293 cells. Supernatants from HEK-293 cells were screened for the presence of CXCR4-binding antibodies and a list of clones of interest was generated. In parallel, V-region sequences were also recovered through NGS. These were cloned into human IgG4-PE constant region vectors and DNA from these vectors was used to transiently transfect HEK-293 cells. Supernatants from these cells were then screened for the presence of functional anti-CXCR4 antibodies.

### Example 4—Primary Screening of B-Cell Expressed Supernatants for Binding to Cell-Expressed CXCR4 Using Mirrorball Screening Technology

Supernatants collected from HEK-293 transfections in Example 3 were screened for binding to CXCR4 or StaR® CXCR4 expressed on the surface of CHO-S cells. Supernatants were plated into non-binding black-walled, clear-bottom tissue culture treated 384-well plates (Corning) at 10 μL per well (/well). Positive (R&D Systems, MAB170-SP) and negative control antibodies (mouse IgG2a, in-house) were added to required wells in PBS (Gibco), 1% BSA (Sigma) with 0.1% sodium azide (Severn Biotech) at a volume of 10 μL/well. Positive and negative control antibodies were added at a concentration of 1.5×10⁻⁷ M to give a final concentration of 3.0×10⁻⁸ M. Cells (CXCR4 or StaR® CXCR4) previously stained and frozen with Cell Trace Red (Life Technologies) were added at 2×10³/well in PBS (Gibco), 1% BSA (Sigma) with 0.1% sodium azide (Severn Biotech) at a volume of 30 μL. Finally, 10 μL of Alexa Fluor 488 labelled goat anti-mouse antibody (Jackson Immuno Research) was added at a concentration of 4 μg/mL to give a final concentration of 0.8 μg/mL. Plates were left to incubate at 4° C. overnight and read on Mirroball (TTP Labtech).

Antibodies were identified by first identifying the cell population (CXCR4 or StaR® CXCR4) through defining the perimeter of the cell size (e.g. 20-200 μm), this allowed exclusion of cell debris and cell clumps etc. Further gating of this population allowed identification of the true stained cell population that were further analysed for AlexaFluor 488 median mean intensity signal to allow ranking of well values to identify antibodies in comparison to positive and negative controls antibodies to give percentage binding value (Equation 1). The selection of antibodies is based on a histogram generated using Genedata Biologics (Genedata) representing the overall distribution of the median mean intensity of the AlexaFluor 488 channel obtained for all the wells. The cut-off (mean median intensity FLU) is visually applied at the tail of the histogram when the frequency of antibodies decreases (Table 2). Antibodies to be carried forward to secondary screening stage were selected from comparing CXCR4 and StaR® CXCR4 binding antibodies to identify common and unique antibodies to each cell line. All common and CXCR4 antibodies were selected along with unique antibodies to StaR® CXCR4 cell lines (Table 2).

$$Calculation of \% binding for primary screening$$

$$\% Binding = ( Median of FL 2 Mean Intensity - NSB ) / ( TB - NSB ) \times 100 \qquad Equation 1$$

Non-Specific Binding (NSB) refers to the averaged value of AlexaFluor 488 media mean intensity of wells with negative control antibody. Total Binding (TB) refers to the averaged value of AlexaFluor 488 media mean intensity of wells with positive control antibody.

TABLE 2
Summary of primary screening results

| | | | Total | Median Mean Cut-off | |
|---|---|---|---|---|---|
| Super- | Positive Hits | | Primary | (FLU) | |
| natants | CXCR4 | CXCR4 | hits | CXCR4 | CXCR4 |
| screened | StaR ® Cells | Cells | selected | StaR ® Cells | Cells |
| 502 | 27 | 42 | 44 | ≥100 | ≥250 |
| 259 | 39 | 78 | 83 | ≥85 | ≥160 |
| 216 | 14 | 8 | 17 | ≥70 | ≥50 |

Summary of number of supernatants screened from immunisations, and number of supernatants meeting primary screening selection criteria for binding to CXCR4 or StaR® CXCR4. Table details number of supernatants screened, number of positive hits and associated cut-off values.

### Example 5—Secondary FACS Screening for Binding to Cell Expressed CXCR4

Supernatants from Example 3 were tested for ability to bind to CHO-S cells expressing CXCR4. CHO-S cells expressing CXCR4 (see Example 1) or wild-type control cells, were diluted in FACS buffer (DPBS (Sigma) 1% BSA (Sigma) 0.1% sodium azide (Severn Biotech)) and were distributed to 96-well, V-bottom plates (Greiner) at a density of 1×10⁵ cells/well. Plates were centrifuged at 300 g for 3 min at 4° C. For supernatant screening, supernatants were aspirated and 25 μL of undiluted supernatant or 25 μL of positive control antibody or negative control antibody were added to the washed cells. Each plate contained specific wells of positive or negative control antibodies at 1×10⁻⁸ M. Positive and negative control antibodies were also used to produce a control titration curve starting at 3×10⁻⁸ M. Cells were incubated at 4° C. for 60 min. Plates were centrifuged at 300 g for 3 min at 4° C. and 100 μL FACS buffer was added to wash the cells. Wash steps were repeated a further two times. To detect antibody binding, 50 μL of goat anti-mouse AlexaFluor 647 (Jackson Immuno Research) diluted to 2 μg/mL in FACS buffer was added to cells. Cells were incubated 4° C. for 60 min. Plates were centrifuged at 300 g for 3 min at 4° C. and 100 μL FACS buffer was added to wash the cells. Wash steps were repeated a further two times. Cells were fixed by addition of 25 μL 4% paraformaldehyde for 20 min at 4° C. Plates were centrifuged at 300 g for 3 min at 4° C. and 100 μL FACS buffer was added to wash the cells. These wash steps were repeated a further two times. Cells were washed once as above and resuspended in 100 μL FACS buffer for analysis. AlexaFluor 647 signal intensity (geometric mean) was measured by flow cytometry using a CytoFLEX (Beckman Coulter).

Using Flow Jo analysis software (Flow Jo LLC) the population of CHO-S cells was gated using the forward and side scatter function. The GeoMean at 647 nm (Alexa Fluor 647 Comp-FL4-A) obtained for the gated CHO-S cells of each well was calculated and then exported to an Excel table (Microsoft). The average of the GeoMean values was calculated for the positive and negative control wells. The binding of each antibody is assessed in comparison to the control wells by calculating their percentage of binding (Equation 2) using Genedata Biologics (Genedata). Wells containing supernatants were ranked based on GeoMean and a cut-off (0% or 5%) applied in comparison to wild type control cells (Table 3).

$$Calculation of \% inhibition of cAMP response$$

$$\% Binding = ( GeoMean ( 647 nm - NSB ) / ( TB - NSB ) \times 100 \qquad Equation 2$$

Non-specific Binding (NSB) refers to the averaged GeoMean value of wells with negative control antibody. Total Binding (TB) refers to the average GeoMean value of wells with positive control antibody.

TABLE 3
Summary of secondary screening results

| | Secondary hits selected for |
|---|---|
| Supernatants screened | functional characterisation |
| 154 | 122 |

Summary of number of supernatants screened from primary screening hits, and number of supernatants meeting secondary screening selection criteria for binding to CXCR4.

### Example 6—Signalling Inhibition (cAMP and Beta Arrestin)

Antibodies identified from secondary screening (Table 3, Example 5) were expressed at small scale (0.1-3 mg) in IgG4-PE format and assessed for ability to inhibit the CXCL12 mediated inhibition of forskolin induced cAMP in BacMam CXCR4 HEK-293 cells. Selected antibodies were also screened through an inhibition assay for beta-arrestin signalling (Thermo Fisher).

(A) Inhibition of CXCL12 Inhibition of Forskolin-Stimulated cAMP for CXCR4—Two Concentration Screening

HEK-293 cells were infected with 1% v/v CXCR4 BacMam virus (Genscript) in the presence of 0.5 mM sodium butyrate for 24 h and seeded at 25,000 cells/well on collagen-coated half area white 96-well plates (Corning). On experiment day, antibodies were diluted 20-fold in Hanks Buffered Saline Solution (HBSS, Sigma) containing 0.1% bovine serum albumin (BSA, Sigma). These were further diluted 10-fold. Antibodies were screened at 500 nM and 50 nM unless antibody stock concentrations were lower than 10 μM. The media was removed from CXCR4-expressing HEK-293 cells prior to addition of 50 μL Phosphate Buffered Saline (PBS, Sigma) to each well. PBS was then removed and replaced with 20 μL antibody dilution in duplicate. Antibodies were incubated for 30 min at 37° C. Forskolin (Sigma) was diluted to 50 μM in HBSS+0.1% BSA containing 2.5 mM 3-isobutyl-1-methylxanthine (IBMX, Sigma). This solution was then used to dilute CXCL12 (Genscript) to 5×EC₈₀ (EC₈₀=5.5 nM) final assay concentration (FAC). 5 μL of 5×EC₈₀ CXCL12 was added to cells and incubated for further 20 min at 37° C. cAMP was then detected using the HiRange cAMP kit (Cisbio). 20×cAMP-d2 and anti-cAMP-cryptate antibody stocks were generated by resuspending the supplied desiccate in 5 mL H2O. These stocks were then diluted 20-fold into kit lysis buffer and 12.4 μL of each detection reagent was added to each well. Cells were placed on a plate shaker at room temperature (RT) for 1 h before reading plates on the PHERAstar FS microplate reader (BMG Labtech) using standard HTRF protocols, with excitation at 335 nm and emissions read at both 620 nm and 665 nm. HTRF ratios were calculated as in Equation 3, and % inhibition calculated as per Equation 4. Technical hits from two concentration screening were determined on a plate-by-plate basis with a hit corresponding to any well which displayed HTRF value lower than the value of the mean EC₈₀ signal with 3×standard deviation of the EC₈₀ control wells for that plate subtracted.

Hits to be progressed were selected based on displaying >80% inhibition at 500 nM. For those antibodies with poorer expression which were tested at concentrations below this, those that were predicted to display good potency based on a theoretical 30 nM concentration response curve were also progressed. Out of 122 secondary hits screened, 22 were progressed for further characterisation.

$$Calculation of HTRF ratios$$

$$HTRF Ratio = ( Emission at 665 nm ) / ( Emission at 620 nm ) \times 10 , 000 \qquad Equation 3$$

$$Calculation of \% inhibition of cAMP response$$

$$\% Inhibition = 100 - ( HTRF ratio test well - HTRF ratio forskolin control ) / ( HTRF ratio EC \_ 80 SDF1a - HTRF ratio forskolin control ) \times 100 \qquad Equation 4$$

(B) Inhibition of CXCL12-Mediated Inhibition of Forskolin-Stimulated cAMP for CXCR4—Full Inhibition Curve Analysis

HEK-293 (Thermo Fisher) cells were infected with 1% v/v CXCR4 BacMam virus in the presence of 0.5 mM sodium butyrate (Sigma) for 24 h and seeded at 25,000 cells/well on collagen-coated half area white 96-well plates (Corning). The next day, antibodies were serially diluted in Hanks Buffered Saline Solution (HBSS) to generate a 10 point, ½ log dilution series (final assay concentration range 500 nM-15 μM). The media was removed from CXCR4-expressing HEK-293 cells prior to addition of 50 μL PBS to each well. PBS was then removed and replaced with 20 μL antibody dilution in duplicate. Antibodies were incubated for 30 min at 37° C. Forskolin was diluted to 50 μM in HBSS+0.1% BSA containing 2.5 mM 3-isobutyl-1-methylxanthine (IBMX). This solution was then used to dilute CXCL12 to 5×EC₈₀ (EC₈₀=5.5 nM) final assay concentration (FAC). 5 μL of 5×EC₈₀ CXCL12 was added to cells and incubated for further 20 min at 37° C. cAMP was then detected using the HiRange cAMP kit (Cisbio). 20×cAMP-d2 and anti-cAMP-cryptate antibody stocks were generated by resuspending the supplied desiccate in 5 mL H₂O. These stocks were then diluted 20-fold into kit lysis buffer and 12.4 μL of each detection reagent was added to each well. Cells were placed on a plate shaker at room temperature (RT) for 1 h before reading plates on the PHERAstar FS microplate reader (BMG Labtech) using standard HTRF protocols, with excitation at 335 nm and emissions read at both 620 nm and 665 nm.

HTRF ratios were calculated as in Equation 3. Data was normalised as per Equation 4. Data was fitted in GraphPad Prism version 7 (GraphPad Prism Software, Inc., San Diego, CA) to a 4-parameter sigmoidal dose-response curve (Equation 5).

$$4 parameter sigmoidal - dose response fit$$

$$Y = Bottom + ( Top - Bottom ) / ( 1 + 10 ⋀ ( ( Log {IC}_{50} - X ) * HillSlope ) \qquad Equation 5$$

where log IC₅₀ is the log concentration (in M) which inhibits the CXCL12 response by 50%, and % maximum inhibition values are the maximal inhibition of CXCL12 (taken from the minimum asymptote of the curve i.e. ‘bottom’). Log IC₅₀ can be converted to IC₅₀ using Equation 6.

IC50(M)=10\hat{ }(log IC50) Equation 6—Calculating IC₅₀ from log IC₅₀

The activity of 5.5 nM (EC₈₀) of CXCL12 on forskolin induced cAMP activity was inhibited by 22 antibodies in IgG4-PE format with nanomolar to subnanomolar potencies in comparison to the human IgG4-PE isotype control (Table 4).

TABLE 4
Summary of number of purified supernatants progressed for
full inhibition curve analysis of inhibition of CXCL12-mediated
inhibition of forskolin-stimulated cAMP for CXCR4.

| | IC₅₀ (nM) ± | % Max Inhibition ± | # |
|---|---|---|---|
| Antibody | SD | SD | repeats |
| CL-82551 | 39 ± 14 | 92 ± 7 | 3 |
| CL-82658 | 23 ± 9 | 98 ± 6 | 3 |
| CL-82583 | 28 ± 10 | 91 ± 8 | 3 |
| CL-82580 | 46 ± 9 | 90 ± 4 | 3 |
| CL-82495 | 27 ± 9 | 96 ± 5 | 3 |
| CL-82455 | 91 ± 37 | 103 ± 12 | 3 |
| CL-82458 | 40 ± 12 | 88 ± 9 | 3 |
| CL-82558 | 29 ± 27 | 94 ± 26 | 3 |
| CL-82577 | 29 ± 10 | 92 ± 7 | 3 |
| CL-82523 | 24 ± 7 | 87 ± 5 | 3 |
| CL-82562 | 99 ± 51 | 92 ± 7 | 3 |
| CL-83083 | 36 ± 14 | 80 ± 4 | 3 |
| CL-83083-2 | 44 ± 14 | 93 ± 4 | 3 |
| CL-82571 | 44 ± 19 | 88 ± 15 | 3 |
| CL-83158 | 68 ± 28 | 91 ± 17 | 3 |
| CL-82517 | 34 ± 9 | 79 ± 16 | 3 |
| CL-82556 | 17 ± 5 | 94 ± 8 | 3 |
| CL-82574 | 21 ± 4 | 96 ± 8 | 3 |
| CL-82541 | 74 ± 37 | 87 ± 18 | 3 |
| CL-82473 | >1000 | — | 3 |
| CL-148729 | 6 ± 4 | 114 ± 10 | 3 |
| CL-148712 | 19 ± 4 | 105 ± 19 | 3 |
| Benchmark 1 | 31 ± 14 | 87 ± 9 | — |
| IgG4-PE | | | |
| Isotype Control | No Inhibition | — | 3 |
| IgG4-PE | | | |

IC₅₀; Mean concentration (nM; nanomolar) of antibody required to give 50% inhibition of activity.
SD; Standard deviation.

### (C) Inhibition of Beta Arrestin Signalling

Anti-CXCR4 antibodies were screened in a functional cell-based reporter gene assay (Invitrogen) and their potency was assessed by their ability to inhibit the recruitment of beta-arrestin induced by CXCL12. Tango CXCR4-bla U20S cells (Invitrogen) were seeded at 10,000 cells/well, 20 uL/well in a 384 well tissue culture treated black plate with clear flat bottom (Corning) and serum-starved for 24 h at 37° C., 5% CO₂ in an assay medium consisting in DMEM supplemented with 1% dialysed FBS (Gibco), 0.1 mM NEAA (Gibco), 25 mM HEPES (Gibco) and 100 U/mL Penicillin/Streptomycin (Invitrogen). 10 uL/well of antibodies diluted at 4× in assay medium (8 points ranging from 160 nM to 2 μM, duplicates, dilution factor of 4) or assay medium were added to the cells and incubated at 37° C., 5% CO₂ for 30 min. Serial dilutions of CXCL12 (Genscript) were prepared in assay medium at 2×(8 dilutions ranging from 1 μM to 61 μM, dilution factor of 4, duplicates) and 20 μL/well were added to cells not treated with antibodies. 10 μL/well of CXCL12 diluted at 4×=120 nM (EC₅₀=30 nM) in assay medium or assay medium were then added to the cells with antibodies. Each plate contained cell-free control wells, negative control wells containing untreated cells and positive control wells containing cells treated with the CXCL12 EC₅₀ as well as a titration of CXCL12 to confirm the EC₅₀ value in each plate. After a 5 h incubation at 37° C., 5% CO2, plates were equilibrated at room temperature for 10 min prior to the addition of 8 μL/well of LiveBLAzer™-FRET Substrate Mixture prepared at 6× according to manufacturer's recommendations (Invitrogen). Plates were incubated in the dark for 60 min at room temperature before measurement of bottom fluorescence with an excitation at 409/20 nm and a detection at 460/40 nm and 530/30 nm using an Envision plate reader (Perkin Elmer).

Fluorescence counts were analysed using Excel and GraphPad Prism (version 7). The average fluorescence was calculated for the cell-free control wells at 460 nm (Average Blue Background) and at 530 nm (Average Green Background). The Average Blue Background was then subtracted from all the raw values of blue fluorescence at 460 nm; the Average Green Background was subtracted from all the raw values of green fluorescence at 530 nm. The values obtained in this way were called Net Blue Fluorescence and Net Green Fluorescence. The Blue/Green Fluorescence ratio was then calculated by dividing the values of Net Blue Fluorescence by the values of Net Green Fluorescence. The Average Negative Ratio corresponds to the average of the Blue/Green Fluorescence ratio values obtained for the negative control wells, which contained untreated cells. Finally, the Response Ratio was calculated for all wells by dividing the values of Blue/Green Fluorescence ratio by the Average Negative Ratio. The Response Ratios obtained for each antibody were then plotted against the concentrations of antibody in GraphPad Prism. Using a non-linear regression, dose response curves for inhibition with a variable slope were drawn and IC₅₀ values were calculated for each antibody (Equation 5). Antibodies were therefore ranked by potency.

The recruitment of beta-arrestin induced by 30 nM of CXCL12 was inhibited by 22 antibodies with nanomolar to subnanomolar potencies in comparison to the human IgG4-PE isotype control (Table 5).

TABLE 5
Summary of number of purified supernatants progressed
for full inhibition curve analysis of inhibition
beta-arrestin recruitment for human CXCR4.

| | IC₅₀ (nM) ± | % Max Inhibition ± | # |
|---|---|---|---|
| Antibody | SD | SD | repeats |
| CL-82551 | 1.56 ± 0.26 | 98.15 ± 3.03 | 3 |
| CL-82658 | 0.35 ± 0.10 | 100.0 ± 3.94 | 3 |
| CL-82583 | 0.17 ± 0.09 | 94.03 ± 13.1 | 3 |
| CL-82580 | 1.15 ± 0.31 | 100.1 ± 1.40 | 3 |
| CL-82495 | 2.08 ± 0.93 | 98.90 ± 4.10 | 3 |
| CL-82455 | 9.12 ± 7.95 | 86.81 ± 15.8 | 3 |
| CL-82458 | 0.31 ± 0.07 | 101.8 ± 3.05 | 3 |
| CL-82558 | 0.07 ± 0.03 | 99.46 ± 2.16 | 4 |
| CL-82577 | 0.24 ± 0.05 | 101.5 ± 2.93 | 3 |
| CL-82523 | 0.08 ± 0.04 | 100.7 ± 1.69 | 4 |
| CL-82562 | 0.52 ± 0.19 | 100.9 ± 3.59 | 3 |
| CL-83083 | 0.43 ± 0.06 | 98.91 ± 1.40 | 2 |
| CL-83083-2 | 0.79 ± 0.41 | 96.86 ± 8.15 | 3 |
| CL-82571 | 1.00 ± 0.23 | 85.61 ± 33.8 | 3 |
| CL-83158 | 1.43 ± 1.30 | 99.25 ± 2.38 | 3 |
| CL-82517 | 0.84 ± 0.45 | 89.56 ± 20.7 | 3 |
| CL-82556 | 0.14 ± 0.11 | 100.6 ± 2.63 | 4 |
| CL-82574 | 0.14 ± 0.11 | 99.46 ± 1.41 | 4 |
| CL-82541 | 0.38 ± 0.14 | 97.15 ± 0.78 | 3 |
| CL-82473 | >120 | 85.01 ± 7.56 | 3 |
| CL-148729 | 0.11 ± 0.00 | 99.60 ± 2.66 | 2 |
| CL-148712 | 0.19 ± 0.03 | 98.42 ± 5.97 | 2 |
| Benchmark 1 | 0.25 ± 0.13 | 100.9 ± 1.13 | 4 |
| (IgG4-PE) | | | |
| Isotype Control | >120 | 10.06 ± 8.53 | 4 |
| IgG4-PE | | | |

IC₅₀; Mean concentration (nM; nanomolar) of antibody required to give 50% inhibition of activity.
SD; Standard deviation.

### Example 7—Inhibition of CXCL12 Binding

Antibodies identified as having effects on inhibiting CXCR4 signalling through cAMP and beta-arrestin signalling were further characterised for CXCL12 inhibition using a TR-FRET cell-based ligand inhibition assay (Cisbio). Anti-CXCR4 antibodies and benchmark antibody were formatted as IgG4-PE antibodies for these experiments. Assay ready terbium-labelled SNAP-CXCR4 HEK-293 cells were purchased from Cisbio (C1TT1CXCR4). 10-point, ½ log antibody dilutions were prepared at 4× final assay concentration in 1× Tag-Lite buffer (TLB, Cisbio) and 5 μL added to white, 384-well proxiplates (PerkinElmer). AlexaFluor 647-CXCL12 (Almac) was diluted to 160 nM in TLB under low light conditions and 5 μL added to each well. Total and non-specific binding (NSB) was determined by AlexaFluor 647-CXCL12 alone and in the presence of 50 μM AMD-3100 respectively. Assay ready cells were thawed into 5 mL TLB and centrifuged at 335×g for 5 min to pellet the cells. The supernatant was removed, and the cell pellet re-suspended to 0.6×10⁶ cells per ml in TLB. 10 μL cells were added to each well under low light conditions and then incubated for 2 h at room temperature. The plate was read on the PHERAstar FS using standard HTRF protocol (excitation at 335 nm and emissions read at both 620 nm and 665 nm).

HTRF ratios were calculated as described in Equation 3, with specific binding calculated as in Equation 7.

$$Calculation of Specific Binding$$

$$\% Specific Binding = ( ( HTRF ratio test well - HTRF ratio NSB ) / ( HTRF ratio total binding - HTRF ratio NSB ) ) \times 100 \qquad Equation 7$$

All antibodies tested were found to inhibit the binding of CXCL12 to CXCR4 expressing cells. Max inhibition ranged from 96±1 to 101±11% with antibody IC₅₀ values varying from 3.92±1.95 to 0.55±0.04 nM (Table 6).

TABLE 6
Summary of number of purified supernatants progressed
for full inhibition curve analysis of inhibition
of CXCL12 binding to human CXCR4.

| | IC₅₀ (nM) ± | % Max Inhibition ± | Number of |
|---|---|---|---|
| Antibody | SD | SD | repeats |
| CL-82523 | 0.55 ± 0.04 | 101 ± 0 | 3 |
| CL-82556 | 0.70 ± 0.15 | 99 ± 0 | 3 |
| CL-82583 | 0.72 ± 0.08 | 98 ± 1 | 3 |
| CL-82577 | 0.92 ± 0.15 | 98 ± 0 | 3 |
| CL-82541 | 1.16 ± 0.09 | 97 ± 0 | 3 |
| CL-82562 | 0.86 ± 0.07 | 100 ± 1 | 3 |
| CL-82455 | 3.68 ± 1.37 | 98 ± 2 | 3 |
| CL-82473 | 1.20 ± 0.06 | 98 ± 7 | 3 |
| CL-82458 | 0.79 ± 0.03 | 99 ± 0 | 3 |
| CL-82658 | 1.04 ± 0.25 | 100 ± 2 | 3 |
| CL-82558 | 0.57 ± 0.01 | 101 ± 2 | 3 |
| CL-83158 | 3.36 ± 1.35 | 101 ± 11 | 3 |
| CL-82574 | 0.63 ± 0.18 | 98 ± 0 | 3 |
| CL-83083 | 1.74 ± 0.60 | 96 ± 1 | 3 |
| CL-83083-2 | 3.92 ± 1.95 | 97 ± 1 | 4 |
| CL-82517 | 1.06 ± 0.42 | 96 ± 2 | 3 |
| CL-82571 | 1.47 ± 0.15 | 96 ± 1 | 3 |
| CL-82580 | 1.41 ± 0.16 | 97 ± 1 | 3 |
| CL-82551 | 1.21 ± 0.23 | 98 ± 1 | 3 |
| CL-82495 | 1.71 ± 0.23 | 100 ± 1 | 3 |
| Benchmark 1 IgG4-PE | 1.34 ± 0.27 | 97 ± 2 | — |
| Isotype Control IgG4-PE | No Binding | No Binding | 3 |

IC₅₀; Mean concentration (nM; nanomolar) of antibody required to give 50% inhibition of activity.
SD; Standard deviation.

### Example 8—Internalisation of Anti-CXCR4 Antibodies on Ramos Cells

Selected CXCR4-specific mAbs in IgG4-PE format and isotype control (IgG4-PE) were tested for their ability to internalise into CXCR4-expressing cells (Ramos; B cell lymphoma cell line, ATCC). Ramos cells were first labelled non-specifically with the IncuCyte™ CytoLight Rapid Green Reagent (Essen BioScience), excess label washed away with FBS and then the cells were seeded at approximately 150,00 cells/well (15 μL/well) into 384 well tissue culture plates. The antibody dilutions were prepared in RPMI+10% FBS. The final assay concentrations of the antibodies were 100 nM, 31.6 nM, 10 nM, 3.17 nM, 1 nM and 0.32 nM. If internalised, molecules are shuttled to the lysosome, where the pH is about 4.5 to 5.0. pHrodo dyes are almost non-fluorescent at neutral pH, however they fluoresce brightly in acidic environments. The Goat Anti-Human IgG, Fcγ Fragment Specific (Jackson ImmunoResearch)—pHrodo Red labelled secondary antibody (in-house) was used at a 3:1 molar ratio with respect to the starting antibody concentration (i.e. final assay concentrations: 300 nM, 94.95 nM, 30 nM, 9.51 nM, 3 nM and 0.96 nM). 15 μL/well of the antibody reagent dilutions were added to the cells and the plates were incubated at 37° C. in the IncuCyte™ S3 Live Cell Analysis System (Essen BioScience) and imaged continuously with a 20× objective for up to 50 h, collecting phase, red and green fluorescence images at regular (˜2 h) intervals.

After incubation, the IncuCyte™ S3 2018A software pack (Essen BioScience) was used for the image analysis. Processing definitions were defined for confluence, green and red objects separately and then the green and red fluorescence total overlap object area (μm² per image) was used to quantify internalisation. The time dependence of the total overlapping signal was plotted in GraphPad Prism and maximum signal (Total Overlap Object Area) determined for each antibody over time.

Antibody internalisation was observed, and this effect was antibody-specific and concentration dependent. The peak of internalisation for all antibodies was observed within the first 960 min (16 h) of cell loading. The ranking of antibodies based on maximum signal values was: CL-82558 (SEQ ID NOs: 60 & 70), Benchmark 1 (IgG4-PE), CL-82458 (SEQ ID NOs: 39 & 49), CL-82658 (SEQ ID NOs: 81 & 91), CL-82574 (SEQ ID NOs: 18 & 28), human anti-CD20 antibody, CL-83083 (SEQ ID NOs: 102 & 112) and human isotype control (IgG4-PE) (FIGS. 1A-1H, Table 7). The antibodies tested were considered to be positive for internalisation.

TABLE 7
Ranking of antibodies based on maximum signal values.

| | Max Signal |
|---|---|
| Antibody | (Total Overlap Object Area) |
| CL-82558 | 23911.3 |
| Benchmark 1 (IgG4-PE) | 21241.4 |
| CL-82458 | 20801.6 |
| CL-82658 | 18925.2 |
| CL-82574 | 18105.8 |
| Positive Control (human | 13916.2 |
| anti-CD20 antibody) | |
| CL-83083 | 9668.6 |
| Isotype Control (IgG4-PE) | 6788.6 |

Signal indicates maximum internalisation of antibody achieved.

### Example 9—Apoptosis of Anti-CXCR4 Antibodies on Jurkat and Primary T-Cells

(A) Assessment of Apoptosis Using Anti-CXCR4 Antibodies with Jurkat Cells

Five anti-CXCR4 antibodies (CL-82558, CL-82458, CL-82658, CL-82574, CL-83083) in IgG4-PE format were tested alongside an appropriate human isotype control antibody (IgG4-PE) and benchmark anti-CXCR4 antibody, Benchmark 1 (in IgG4-PE format) in a primary naïve T-cell apoptosis assay using Jurkat cells (an immortalised T-cell line).

Antibodies were diluted in RPMI media (Gibco) containing 10% v/v foetal bovine serum (FBS) (complete RPMI) to generate a 10-point ½ log dilution series at 2× final assay concentration. Anti-Fas antibody clone CH11 (Millipore) was diluted to 200 nM and acted as a positive control for apoptosis, while IgM and IgG4-PE isotype controls acted as negative controls. 100 μL antibody dilutions were added to 96-well full area flat-bottomed plate (Corning). Jurkat cells were diluted to 1×10⁶ cells per mL in complete RPMI and 100 μL cells added to every well. The plate was then incubated for 24 h at 37° C., 5% CO₂.

The next day, annexin buffer was prepared by diluting 5× annexin buffer (Thermo Fisher) 5-fold in water. 10 μg/mL propidium iodide (PI) working solution was prepared by diluting 1 mg/mL stock solution (Thermo Fisher) 100-fold in annexin buffer. Cells were re-suspended and transferred to a V-bottomed 96-well storage plate (Corning). The plate was centrifuged at 300×g for 5 min to pellet cells. The supernatant was carefully removed, and cells resuspended in 100 μL cold PBS. The plate was then centrifuged again (300×g, 5 min). The supernatant was carefully removed, and the cells resuspended in 85 μL annexin buffer. 5 μL AF647-Annexin V (Thermo Fisher) and 10 μL of 10 μg/mL PI solutions were added to every well, except for single stain control wells used for setting up compensation controls in the flow cytometer and the gates. Cells were stained for 15 min at room temperature in the dark.

Following staining, cells were analysed for apoptosis by flow cytometry using a FACS Canto II (BD BioSciences). Cells in early apoptosis showed positive AF647-Annexin V staining while cells in late stage apoptosis were positive for both PI and AF647-Annexin V staining.

Measurements of apoptosis induction are expressed as the mean±S.D. of the SIA (Equation 8) expressed as a % of the anti-Fas-induced apoptosis (considered to be 100%) and are based on the Annexin V+ staining from 3 independent experiments performed in duplicate.

Anti-CXCR4 antibodies (CL-82558, CL-82458, CL-82658, CL-82574, CL-83083) and isotype control (IgG4-PE) did not show a concentration-dependent increase in apoptosis; the maximal % specific-induced apoptosis (SIA; expressed as % of anti-Fas response) was measured at −4.9-1.4%. In contrast, Benchmark 1 (IgG4-PE) showed a concentration-dependent increase in apoptosis with a maximum % SIA response of 34%. Data is summarised in Table 8.

TABLE 8
Apoptosis of Jurkat T-cells induced by soluble
anti-CXCR4 antibodies and controls.

| Antibody | % SIA | S.D | n |
|---|---|---|---|
| No antibody - Jurkat cells | 0 | 0.0 | 3 |
| Anti-Fas positive control | 100 | 0 | 3 |
| Isotype control (IgG4-PE) | 0 | 0 | |
| Benchmark 1 (IgG4-PE) | 33.9 | 2.4 | 3 |
| CL-82558 | −1.9 | 3.4 | 2 |
| CL-82458 | −4.9 | 5.6 | 3 |
| CL-82574 | 1.4 | 3.5 | 3 |
| CL-82658 | −4.1 | 5.8 | 3 |
| CL-83083 | −1.7 | 3.1 | 3 |

SIA; specific-induced apoptosis.
S.D; standard deviation.
n; number of assay repeats.

(B) Assessment of Apoptosis Using Anti-CXCR4 Antibodies with Primary Naïve T-Cells

Five anti-CXCR4 antibodies (CL-82558, CL-82458, CL-82658, CL-82574 and CL-83083) in IgG4-PE format were tested alongside an appropriate isotype control antibody IgG4-PE and benchmark anti-CXCR4 antibody Benchmark 1 (IgG4-PE) in a primary naïve T-cell apoptosis assay. Antibody-induced apoptosis of naïve human T-cells was assessed by Annexin V binding and cell permeability to a live/dead dye by flow cytometry.

### (i) Isolation of Primary Naïve T-Cells

Peripheral blood mononuclear cells (PBMCs) were isolated from leukocyte cones prepared from healthy human donor blood and covered by Research Ethics Committee approval. Primary human T-cells were isolated from PBMCs by negative selection using the pan T-cell isolation kit (Stemcell Technologies), according to the manufacturer's instructions.

### (ii) Primary Naïve T-Cell Apoptosis Assay Using Soluble Antibodies

Naïve T-cells were seeded at 5×10⁵ cells/well into 96-well flat-bottomed plates (Corning Inc.) in 100 μL of RPMI+1% FCS (complete medium). The five anti-CXCR4 antibodies or appropriate isotype control (IgG4-PE) antibody, and X-linker Affinipure (Fab′)₂ fragment goat anti-human IgG Fc fragment specific antibody (Jackson ImmunoResearch), added to this assay to promote antibody cross-linking, were diluted in complete medium at a 1:1 molar ratio and titrated using serial 3-fold dilutions to give a final concentration range of 4.44-120 nM. Antibodies were added at 2× concentration in 100 μL/well complete medium to duplicate wells of T-cells and the cells incubated overnight (˜18 h) at 37° C., 5% CO₂. As a positive control for apoptosis induction, staurosporine (Abcam) was added at a final concentration of 1 μM to control wells in duplicate. The following day, the cell suspensions were recovered from the assay plate and transferred to a deep-well V-bottomed 96-well plate (Thermo Fisher) for staining with Annexin V and live/dead fixable yellow dead cell stain (both Thermo Fisher). The cells were pelleted by centrifugation at 350×g for 10 min at 4° C. and then washed once with 200 μL/well of 1× Annexin V binding buffer (prepared from a 5× concentrate; Thermo Fisher). The cells were then resuspended in 100 μL of 1× Annexin V binding buffer containing 5 μL of Annexin V Alexa Fluor 488 conjugate and 1 μL of 1:10 diluted live/dead fixable yellow dead cell stain and incubated for 15 min at room temperature, protected from the light. T-cells were also stained in duplicate with Annexin V Alexa Fluor 488 conjugate or live/dead fixable yellow dead cell stain alone, to prepare single stain controls for compensation. At the end of the incubation, 200 μL of 1×Annexin V binding buffer was added to each well and the plate centrifuged at 350×g for 10 min at 4° C. The cells were fixed by resuspending in 100 μL/well of 4% paraformaldehyde (PFA)/PBS (Affymetrix) and incubating for 15-20 min at room temperature. Cells were pelleted by centrifugation at 400×g for 10 min at 4° C., and then resuspended in 300 μL/well of 1×Annexin V binding buffer for analysis on the Attune NxT Flow Cytometer.

Measurements of apoptosis induction are expressed as the mean+/−S.D. of the specific-induced apoptosis (SIA) (Equation 8) expressed as a % of the staurosporine-induced apoptosis (considered to be 100%) and are based on the Annexin V+ staining from duplicate samples.

$$Calculation of specific - induced apoptosis ( SIA )$$

$$\% SIA - ( ( Compound induced - spontaneous ) / ( 100 - spontaneous ) ) \times 100 \qquad Equation 8$$

SIA=specific induced apoptosis
Compound induced=apoptosis induced by anti-CXCR4 antibody, human IgG4-PE isotype control or staurosporine
Spontaneous=background apoptosis in T-cells from wells receiving medium only
   (iii) Primary Naïve T-Cell Apoptosis Assay Using Plate-Bound Antibodies

Anti-CXCR4 antibodies, or appropriate isotype control antibody human IgG4-PE, were serially diluted 1:3 in PBS to give a final concentration range of 4.44-120 nM antibody. Aliquots of 100 μL were added to duplicate wells on a 96-well EIA/RIA flat-bottomed high bind microplate (Corning Inc.) and incubated for at least 1 h at room temperature. The antibodies were then aspirated from the wells and freshly isolated T-cells seeded at 5×10⁵ cells/well in 200 μL assay medium onto the antibody pre-coated plate. The cells were incubated overnight with the plate-bound antibodies (˜18 h) at 37° C., 5% CO₂. The following day, the cells were recovered from the assay plate by vigorous pipetting up and down and transferred to a deep-well V-bottomed 96-well plate for staining with Annexin V and live/dead fixable yellow dead cell stain and analysis of apoptosis, as described before (section b).

No significant induction of apoptosis was observed for any of the five anti-CXCR4 antibodies or benchmark antibody Benchmark 1 when used in a soluble format with a cross-linker. Apoptosis induced using plate-bound antibody was lower for all anti-CXCR4 leads than observed for Benchmark 1 (IgG4-PE), which induced 7-21% apoptosis of T-cells. The % SIA (expressed as % of Staurosporine-induced apoptosis) at the highest antibody concentration tested (120 nM) is summarised for different T-cell donors for soluble+ cross-linker (Table 9) and plate-bound (Table 10) antibodies.

TABLE 9
Apoptosis induced by soluble anti-
CXCR4 antibodies with a cross-linker.

| Treatment | % induced apoptosis | | | |
|---|---|---|---|---|
| T-cell donor | D0441 | D0448 | D0455 | D0464 |
| T-cells alone | 0.5 | 0.0 | −0.1 | 0.3 |
| Staurosporine | 100 | 100 | 100 | 100 |
| Isotype control (IgG4-PE) | −0.5 | −1.5 | −0.8 | 0.0 |
| Benchmark 1 IgG4-PE | 3.2 | −1.7 | 0.5 | 2.1 |
| CL-82558 | N.d. | N.d. | 0.5 | 1.5 |
| CL-82458 | N.d. | N.d. | −0.3 | 0.1 |
| CL-82574 | N.d. | N.d. | 0.4 | 0.7 |
| CL-82658 | N.d. | N.d. | 0.9 | 0.1 |
| CL-83083 | N.d. | N.d. | −1.1 | 0.9 |

N.d.; not determined.

TABLE 10
Apoptosis induced by plate-bound anti-CXCR4 antibodies.

| Treatment | % induced apoptosis | | | |
|---|---|---|---|---|
| T-cell donor | D0441 | D0448 | D0455 | D0464 |
| T-cells alone | −1.1 | −1.0 | 0.3 | 0.3 |
| Staurosporine | 100 | 100 | 100 | 100 |
| Isotype control (IgG4-PE) | −0.1 | −0.6 | 1.4 | −0.1 |
| Benchmark 1 (IgG4-PE) | 7.9 | 6.8 | 20.6 | 15.4 |
| CL-82558 | N.d. | N.d. | 7.0 | 3.6 |
| CL-82458 | N.d. | N.d. | 2.4 | 3.6 |
| CL-82574 | N.d. | N.d. | 8.6 | 5.4 |
| CL-82658 | N.d. | N.d. | 6.4 | 7.7 |
| CL-83083 | N.d. | N.d. | 3.8 | 7.4 |

N.d.; not determined.

### Example 10—Cynomolgus Cross-Reactivity

### (A) Binding of Anti-CXCR4 Antibodies to Cynomolgus Monkey Expression Cells

Anti-CXCR4 antibodies (CL-82558, CL-82458, CL-82658, CL-82574, CL-83083 & CL-148729) in IgG4-PE format were tested alongside an appropriate isotype control antibody (IgG4-PE) and benchmark anti-CXCR4 Benchmark 1 in IgG4-PE format for binding to cells expressing cynomolgus monkey CXCR4 (FIG. 2). Cynomolgus monkey CXCR4 expressing CHO-S cells were plated at 100 uL/well of cells at 1 million of cells/mL (0.1 million of cells/well) into a 96 well V-bottom plate (Greiner). Plates were centrifuged at 300×g for 3 min at 4° C. and supernatant discarded. Antibodies were prepared in a separate 96 well v-bottom plate to give 8 dilution points with a dilution factor of 5. Each antibody had a top concentration of 120×10 nM and were diluted in FAGS buffer PBS (Gibco), 1% BSA (Sigma) with 0.1% sodium azide (Severn Biotech). Primary antibody dilutions were added to cells (50 μL/well) and cell pellets resuspended by pipetting. Plates was incubated for 1 h at 4° C. 100 μL/well of PBS, was added and plates centrifuged at 300×g for 3 min at 4° C. and supernatant discarded. This step was repeated twice. 2 μg/mL anti-human Alexa Fluor 647 secondary detection antibody (Jackson Immuno Research) was added at 50 μL/well diluted in FACS buffer. Cells were resuspended by pipetting. Plates were incubated for 1 h at 4° C. 100 μL/well PBS, was added and plates centrifuged at 300×g for 3 min at 4° C. and supernatant discarded. This step was repeated twice. Finally, cells were resuspended in 100 μL/well of PBS and analysed on CytoFLEX (Beckman Coulter).

Binding of antibodies to cynomolgus CXCR4 expressed on CHO-S cells was analysed by gating the main cell population using forward and side scatter (Flow Jo, Flow Jo LLC) to allow for doublet discrimination. Comp-FL4-A Geomean (Alexa 647) values obtained for each well were exported to Excel (Microsoft) as a table and values plotted using GraphPad Prism to produce titrations curves. EC₅₀ values were calculated by transforming the data and fitting a 4-parameter logistic regression curve to each antibody titration.

Five antibodies bind to cynomolgus monkey CXCR4 expressing cells with nanomolar affinity (1.11 nM to 11 nM) (Table 11). CL-148729 does not bind to cynomolgus monkey CXCR4 (FIG. 2). Benchmark 1 IgG4-PE bind to cynomolgus monkey CXCR4 expressing cells.

TABLE 11
EC₅₀ values of antibodies binding to CHO cell expressed
cynomolgus CXCR4 as measured by flow cytometry.

| | | | Number |
|---|---|---|---|
| Antibody | EC₅₀ (nM) | SD (nM) | of repeats |
| CL-82558 | 1.1 | 0.36 | 3 |
| CL-82458 | 2.2 | 0.79 | 3 |
| CL-82574 | 3.5 | 1.3 | 3 |
| CL-82658 | 4.2 | 2.7 | 3 |
| CL-83083 | 11.0 | 3.1 | 2 |
| CL-148729 | No Binding | No Binding | 3 |
| Benchmark 1 IgG4-PE | 12.2 | 8.9 | 3 |
| Isotype Control IgG4-PE | — | — | 3 |

Values are shown as average EC₅₀ from three independent experiments.
SD; standard deviation.
NB; no binding.

(B) Inhibition of CXCL12-Mediated Inhibition of Forskolin-Stimulated cAMP for Cynomolgus CXCR4

Anti-CXCR4 antibodies (CL-82558, CL-82458, CL-82658, CL-82574 and CL-83083) in IgG4-PE format were tested alongside a human isotype control antibody (IgG4-PE) and benchmark anti-CXCR4 antibody Benchmark 1 in IgG4-PE format in a cynomolgus monkey CXCR4 cAMP signalling assay.

HEK-293 cells were infected with 5% v/v cyno CXCR4 BacMam virus for 24 h in the presence of 0.5 mM sodium butyrate in 120 mm² tissue culture dishes. On experiment day, antibodies were diluted to 4×starting concentration in HBSS+0.1% BSA and 10-point, ½ log concentration response curves generated. 2 μL antibody was added to 384-well proxiplate (PerkinElmer). Cells were washed with PBS prior to addition of cell dissociation solution (Gibco). Cells were then collected into PBS, pelleted by centrifugation (335×g, 5 min,) and the supernatant removed. Cells were then re-suspended in HBSS+0.1% BSA to 0.33×10⁶ cells per mL and 6 μL added to each well. Plates were incubated at 37° C. for 45 min.

Forskolin was diluted to 5 μM in HBSS+0.1% BSA containing 2.5 mM IBMX. This was then used to dilute CXCL12 to 5×EC₅₀ FAC (EC₅₀=0.74 nM). 2 μL CXCL12 was then added to plates following antagonist incubation and incubated for a further 20 min at 37° C. The cAMP level was detected using the HiRange cAMP kit (Cisbio). 20×cAMP-d2 and anti-cAMP-cryptate antibody stocks were generated by resuspending the supplied desiccate in 5 mL H₂O. These stocks were then diluted 20-fold into kit lysis buffer and 12.4 μL of each detection reagent was added to each well. Cells were placed on a plate shaker at room temperature (RT) for 1 h before reading plates on the PHERAstar FS microplate reader (BMG Labtech) using standard HTRF protocols, with excitation at 335 nm and emissions read at both 620 nm and 665 nm. HTRF ratios were calculated as in Equation 4, and % inhibition and IC₅₀ values calculated as per Equations 4 and 5.

All five antibodies tested inhibit cynomolgus CXCL12-mediated inhibition of forskolin-stimulated cAMP. Percentage maximum inhibition varied from 78±10 to 90±11% with IC₅₀ values in the range of 1.0±0.8 to 11.8±7.8 nM (Table 12).

TABLE 12
Inhibition of cynomolgus CXCL12-mediated
inhibition of forskolin-stimulated cAMP.

| | IC50 (nM) ± | % Maximum | Number |
|---|---|---|---|
| Antibody | SD | Inhibition ± SD | of repeats |
| CL-83083 | 11.8 ± 7.8 | 84 ± 19 | 4 |
| CL-82574 | 1.2 ± 0.6 | 90 ± 11 | 4 |
| CL-82558 | 1.3 ± 0.7 | 83 ± 5 | 4 |
| CL-82458 | 2.0 ± 0.7 | 78 ± 10 | 4 |
| CL-82658 | 1.0 ± 0.8 | 80 ± 16 | 4 |
| Benchmark 1 IgG4-PE | 12.4 ± 5.4 | 92 ± 23 | 4 |
| Isotype Control IgG4-PE | No inhibition | — | 3 |

IC₅₀; Mean concentration (nM; nanomolar) of antibody required to give 50% inhibition of activity.
SD; Standard Deviation.

### Example 11—Affinity Determination by Surface Plasmon Resonance (SPR)

### (A) Binding of Fab Fragment Antibodies to CXCR4 Expressed on Virus Like Particles

Fab apparent affinities for CXCR4 were generated by SPR using the Biacore 8K (GE) (Table 13). Biotinylated CXCR4-virus like particle (VLP, Lot #INT-1793B, Integral Molecular) were captured on the active flow channel of a SA chip (GE #BR100531). Five purified anti-CXCR4 Fab fragments CL-82558, CL-82458, CL-82574, CL-82658 and CL-83083 were used as analytes and flown over captured CXCR4 VLP at 1.23 nM, 3.7 nM, 11.11 nM, 33.33 nM and 100 nM. Binding sensorgrams were double referenced using a buffer injection (i.e. 0 nM), and the data was fitted to the 1:1 model inherent to the Biacore 8K evaluation software. The assay was run at 25° C. and using 10 mM Hepes (Sigma H0887), 150 mM NaCl (Sigma S5150), 0.1% w/v BSA (Sigma A4503), 7.4 pH as running buffer. None of the Fabs were binding VLP-Null (lacking hCXCR4).

TABLE 13
Affinity measurements (K_{D}) of anti-CXCR4 fab
binding to CXCR4 expressed on VLP particles.

| Fab | Ligand | ka (M⁻¹s⁻¹) | kd (1/s) | K_{D} (nM) |
|---|---|---|---|---|
| CL-82558 | hCXCR4 VLP | 2.32E+06 | 1.55E−03 | 0.67 |
| CL-82458 | hCXCR4 VLP | 8.87E+05 | 1.11E−03 | 1.26 |
| CL-82574 | hCXCR4 VLP | 9.27E+05 | 4.81E−04 | 0.52 |
| CL-82658 | hCXCR4 VLP | 6.16E+05 | 4.23E−04 | 0.69 |
| CL-83083 | hCXCR4 VLP | 5.46E+05 | 1.02E−03 | 1.86 |

K_{D} calculated in nanomolar (nM) concentration units.

### (B) Binding of Anti-CXCR4 Antibodies to StaR® CXCR4 Expressing Nanodiscs

Antibody affinities for CXCR4 were estimated by SPR using the Biacore T200 instrument (GE Healthcare) (Table 14). Anti-IgG antibodies were immobilised on chip CM5 (GE Healthcare) by amine coupling. Anti-CXCR4 antibodies (CL-82558, CL-82458, CL-82658, CL-82574, CL-83083) in IgG4-PE format were captured in the active channel. A channel with no anti-CXCR4 antibodies captured was used as a reference. CXCR4 StaR® nanodiscs were then injected at a concentration of 0 (blank) and 250 nM. The blank-subtracted sensorgrams were fitted to the 1:1 interaction model using the Biacore T200 evaluation software. The assay was run at 25° C. using 0.01 M HEPES pH 7.4, 0.15 M NaCl as running buffer.

TABLE 14
Affinity measurements (K_{D}) of anti-CXCR4 antibodies binding
to StaR ® CXCR4 expressed on nanodiscs.

| Sample | kₐ (M⁻¹s⁻¹) | | K_{d} (1/s) | | K_{D} (nM) | |
|---|---|---|---|---|---|---|
| Ab | N = 1 | N = 2 | N = 1 | N = 2 | N = 1 | N = 2 |
| CL-82558 | 1.2E+05 | 7.7E+04 | 4.7E−04 | 1.3E−04 | 3.9 | 1.7 |
| CL-82458 | 6.1E+04 | — | 4.6E−04 | — | 7.5 | — |
| CL-82574 | 1.1E+05 | — | 8.8E−04 | — | 8.0 | — |
| CL-82658 | 5.2E+04 | — | 5.0E−04 | — | 9.6 | — |
| CL-83083 | 1.4E+05 | — | 9.7E−04 | — | 7.1 | — |

### Example 12—CXCR7 Cross-Reactivity

CXCR7 expressing CHO-S cells were plated at 100 μL/well of cells at 1 million of cells/mL (0.1 million of cells/well) into a 96 well V-bottom plate (Greiner). Plates were centrifuged at 300×g for 3 min at 4° C. and supernatant discarded. Antibodies (including CL-82558, CL-82458, CL-82658, CL-82574 & CL-83083 in IgG4-PE format and controls) were prepared in a separate 96 well v-bottom plate to give 8 dilution points with a dilution factor of 5. Each antibody had a top concentration of 120 nM and were diluted in FACS buffer PBS (Gibco), 1% BSA (Sigma) with 0.1% sodium azide (Severn Biotech). Primary antibody dilutions were added to cells (100 μL/well) and cell pellets resuspended by pipetting. Plate was incubated for 1 h at 4° C. 100 μL/well of PBS, was added and plates centrifuged at 300×g for 3 min at 4° C. and supernatant discarded. This step was repeated twice. 2 μg/mL of anti-human or anti-mouse Alexa Fluor 647 secondary detection antibody (Jackson Immuno Research) was added at 50 μL/well diluted in FACS buffer. Cells were resuspended by pipetting. Plate was incubated for 1 h at 4° C. 100 μL/well of PBS, was added and plates centrifuged at 300×g for 3 min at 4° C. and supernatant discarded. This step was repeated twice. Finally, cells were resuspended in 100 μL/well of PBS and analysed on CytoFLEX (Beckman Coulter).

Binding of antibodies to CXCR7 expressed on CHO-S cells was analysed by gating the main cell population using forward and side scatter (Flow Jo, Flow Jo LLC) to allow for doublet discrimination. Comp-FL4-A Geomean (Alexa 647) values obtained for each well were exported to Excel (Microsoft) as a table and values plotted using GraphPad Prism to produce titrations curves.

Apart from positive control (mAb #11G8 R&D Systems), no antibodies were shown to bind to CXCR7 expressing CHO-S cells (FIG. 3).

### Example 13—Inhibition of CXCL12 Stimulated Response for Isolated Human T-Cells Using Label Free Dynamic Mass Redistribution (DMR) Assay

The Corning™ Epic™ technology is a label free optical biosensor system for detection of ligand-induced changes within the cell (dynamic mass redistribution, DMR). In brief, polarized light is passed through the bottom of a biosensor microtiter plate containing the cell samples, and a shift in wavelength of reflected light is indicative of redistribution of cellular constituents triggered upon receptor activation. The wavelength shift may vary in magnitude, direction (positive or negative) and over time depending on how different activated signalling pathways cause various intracellular molecules to relocate.

Cells are allowed to settle to the bottom of a well equipped with an optical biosensor. Ingoing broadband light is directed to travel along the bottom. The electromagnetic field extends into the cell layer for a depth of about 150 nm and loses energy depending on the optical density of the adjacent cell area, and the outgoing wavelength is measured. GPCR-mediated signalling causes changes within the cell that affect optical density and thereby shifts the outgoing wavelength (measured in picometers) relative to pre-stimulus condition and is recorded over run time of the assay. The magnitude of this wavelength shift is proportional to the amount of relocated intracellular matter: an increase in mass contributes positively and a decrease negatively to the overall response.

(A) Isolation of Primary T-Cells from Human Whole Blood

Fresh human blood was provided by Cambridge Bioscience. 150-200 mL of blood containing anticoagulant (CPD, citrate phosphate dextrose) was used. Initially the blood was diluted with cold Dulbeccos PBS (Sigma) in 1:2 ratio. 30 mL of diluted blood were carefully layered into 50 mL Falcon tubes containing 15 mL Lymphopure (Biolegend). All tubes were centrifuged at 800×g for 30 min at room temperature without braking. After the centrifugation cells from the buffy coat layer, the layer located just above the Lympopure and under the plasma interface, were collected using a Pasteur pipette. Collected cell suspension was diluted with Dulbeccos PBS in half and centrifuged at 335×g for 10 min at room temperature. All cell pellets were pulled together into one 50 mL Falcon tube and diluted in Dulbeccos PBS, then counted and centrifuged again at 335×g for 10 min. After this step, obtained PBMCs (peripheral blood mononuclear cells) were used to isolate T-cells using the pan T-cell isolation kit (Miltenyi Biotec). Cell pellet was resuspended in the volume of buffer (Dulbeccos PBS without calcium or magnesium (Sigma)), with 0.5% BSA (Sigma), and 2 mM EDTA (Sigma) calculated as 40 μL per 1×10⁷ cells. Then, 10 μL per 1×10⁷ cells of pan T-cell biotin-antibody cocktail was added, mixed well and incubated on ice for 5 min. Cell suspension was further diluted with buffer, 30 μL per 1×10⁷ cells prior to addition of 20 μL per 1×10⁷ cells of pan T-cell microBead cocktail, mixed well and incubated on ice for 10 min. Next LS column (Mitenyi Biotec) was inserted into the magnetic field of QurdoMacs separation unit (Mitenyi Biotec) and rinsed with 3 mL of buffer. The cell suspension was applied onto the column and the flow-through containing the enriched T-cells was collected. The column was rinsed with 3 mL of buffer two more times collecting the flow-through into the same tube. This enriched T-cells fraction was resuspended, and the number of cells was counted. A small fraction of cells was taken out to confirm the purity of T-cells by staining the cells with anti CD3 PE fluorophore conjugated antibody (Becton Dickinson) and analysing by flow cytometry using a FACS Canto II (BD BioSciences). Typically, the purity of T-cells was greater than 96%. The remaining cell suspension was centrifuged at 335×g for 5 min and the cells were resuspended in serum free medium (Gibco).

### (B) DMR Assay Using EPIC BT System Analyser

Isolated T-cells re-suspended in serum free medium were plated at 150,000 cells/well per 45 μL into 384-well glass bottom EPIC plate (Corning). Cells were left in the incubator at 37° C. for 2 h to settle. Then, the plate was placed into EPIC BT System analyser (Corning) to obtain background reading for 2 min. The assay was performed at 37° C. Test antibody dilutions were prepared in serum free medium to achieve 10-point half log dilutions at 10×final assay concentration, typically ranging from 0.2 μM to 6.7 μM final assay concentrations. The plate readings were paused and, either 5 μL of antibody dilutions or 5 μL of controls, were added to the cells and the changes in the dynamic mass redistribution (DMR) readings were collected every 30 see for 30 min. Then, a second reading was initiated. After the baseline reading was recoded, the assay was paused and 5 μL of 10×EC₈₀ concentration CXCL12 was added to all wells apart from control wells, where 5 μL of serum free medium was added. Plate readings were collected again for further 60 min.

DMR traces over the run time of the assay were analysed using EPIC analyser software by the quantification of the minimal response peak between 5 and 50 min after compound addition. Data was corrected by subtraction of the corresponding buffer controls and exported for further analysis. This data was normalised to the assay controls to calculate % inhibition using Equation 9, where CXCL12 EC₈₀ control was used to define 0% inhibition and unstimulated cells in serum free medium as 100% inhibition.

$$Calculation of \% inhibition$$

$$\% of Inhibition = 100 - ( ( sample well - CXCL 12 EC 80 control ) / ( unstimulated cells control - CXCL 12 EC 80 control ) ) \times 100 \qquad Equation 9$$

IC₅₀ values were derived by fitting data to a 4-parameter logistic function using GraphPad Prism 7.

All anti-CXCR4 antibodies (CL-82458, CL-82558, CL-82658, CL-83083, CL-83083-2 and CL-82574) were able to cause a concentration-dependent reduction in CXCL12 response in primary naïve T-cells with potencies between 1.54 and 12.09 nM. All anti-CXCR4 antibodies tested reduced CXCL12 signalling to between 0-5.1% of the CXCL12 response (Table 15).

TABLE 15
Summary IC₅₀ of anti-CXCR4 antibodies inhibition to
CXCL12 response in primary naïve T-cells.

| | | | Number |
|---|---|---|---|
| Antibody | IC₅₀ (nM) ± SD | % effect ± SD | of repeats |
| CL-82458 | 12.08 ± 3.36 | 2.0 ± 6.7 | 4 |
| CL-82558 | 1.72 ± 0.99 | 4.3 ± 1 | 4 |
| CL-82658 | 1.54 ± 0.32 | −0.6 ± 6.4 | 4 |
| CL-83083 | 3.58 ± 1.86 | 5.1 ± 6.2 | 4 |
| CL-83083-2 | 3.98 ± 1.35 | 4.4 ± 3.9 | 3 |
| CL-82574 | 2.09 ± 1.19 | 0.68 ± 6.3 | 4 |
| Benchmark 1 IgG4-PE | 9.61 ± 1.68 | 11.4 ± 4.7 | 6 |
| IgG4-PE Isotype Control | No Inhibition | — | 8 |

IC₅₀; Mean concentration (nM; nanomolar) of antibody required to give 50% inhibition of activity.
SD; Standard deviation.

### Example 14—Inhibition of Chemotaxis of Jurkat T-Cells or Primary Human T-Cells to CXCL12

### (A) Inhibition of Chemotaxis of Jurkat T-Cells Cells Towards CXCL12

The antagonistic effect of the five anti-CXCR4 antibodies (CL-82558, CL-82458, CL-82658, CL-82574 & CL-83083) in IgG4-PE format, benchmark antibody Benchmark 1 (in IgG4-PE format) and an IgG4-PE isotype control were further investigated in a chemotaxis assay using human T-cells expressing endogenously CXCR4, the Jurkat cells by assessing the ability of antibodies to inhibit the migration of Jurkat cells towards 30 nM of CXCL12 (estimated EC₅₀) using the IncuCyte™ S3 chemotaxis assay system.

The top and bottom sides of chemotaxis plates membranes (Essen BioScience) were coated respectively with 20 and 150 μL/well of Matrigel growth factor reduced (Corning) diluted at 50 μg/mL in RPMI 10% FBS (Invitrogen). Serial dilutions of antibodies were prepared at 2×(8 dilutions ranging from 200 nM to 1.52 μM, dilution factor of 4, triplicates) in assay medium, RPMI+0.5% FBS (Invitrogen) in a 96 well PS V-bottom plate (Greiner). Jurkat cells were diluted in assay medium at 0.166 million of cells per mL. 40 μL/well of antibodies at 2×were mixed by up-and-down pipetting with 40 μL/well of Jurkat cells at 0.166 million of cells per mL in a new 96 well PS V-bottom plate (Greiner). After 30 min at 37° C., followed by 30 min at room temperature, matrigel was removed from both sides of the chemotaxis plates membranes, 200 μL/well of DPBS (Invitrogen) were added to the bottom side of the chemotaxis plates (also called reservoir plates) while 60 μL/well of Jurkat cells with antibodies were transferred to the top side of the chemotaxis plates. Serial dilutions of CXCL12 were prepared in assay medium at 1×(8 dilutions ranging from 1.6 μM to 0.73 nM, dilution factor of 3, triplicates) along with a solution of CXCL12 at its estimated EC₅₀ (30 nM) in a 96 deep well PP V-bottom plate (Corning). Each chemotaxis plate contained negative control wells in which untreated cells were migrating towards assay medium, positive control wells in which untreated cells were migrating towards 30 nM of CXCL12 and a titration of CXCL12 to confirm its EC₅₀ value. After a 45 to 60 min incubation at room temperature, the top side of the chemotaxis plates containing cells and antibodies was transferred on top of a new reservoir chemotaxis plate containing 200 μL/well of CXCL12 or assay medium. Chemotaxis plates were then incubated in the IncuCyte™ S3 Live Cell Analysis System (Essen BioScience) at 37° C. 5% CO2. After a 20 min equilibration, plates were scanned every 3 h for 48 h using a 10×objective in the phase channel and the Chemotaxis Top/Bot scan mode.

Scans of each time point for all the wells were analysed using a Chemotaxis Migration (Top Only) analysis definition on the IncuCyte™ software. Cell masks were defined with an 0.2 segmentation adjustment, an 8-pixel pore size, applying a minimal area filter of 150 μm² and a whole well processing keep-out of 50 pixels. Using the Graph Metrics on the IncuCyte™ software, the Total Phase Object Area Normalized to Initial Top Value [Top], representing the cell confluency in the top compartment of the chemotaxis plate normalized to the initial cell confluency (Top) at T=0 h when the migration assay was started, were retrieved for all the wells at the scan time of 30 h and exported to be further analysed with Excel (Microsoft) and GraphPad Prism (version 7). The exported data were normalised using the average confluency at 30 h obtained in the positive and negative control wells; the migration inhibition percentages resulting from this normalisation were plotted against the antibody concentrations to assess the antibody inhibitory effect on the migration of Jurkat cells induced by CXCL12 (FIG. 4).

The migration of Jurkat cells towards 30 nM of CXCL12 was inhibited by all five anti-CXCR4 antibodies with nanomolar to subnanomolar potencies in comparison to the human IgG4-PE isotype control (Table 16, FIG. 4).

TABLE 16
IC₅₀ values (molarity) of antibodies inhibiting the
migration of Jurkat cells towards 30 nM of CXCL12.

| | IC₅₀ (nM) | IC₅₀ (nM) | IC₅₀ (nM) | Mean IC₅₀ | SD IC₅₀ |
|---|---|---|---|---|---|
| Antibodies | n = 1 | n = 2 | n = 3 | (nM) | (nM) |
| CL-82558 | 0.72 | 0.94 | 0.71 | 0.79 | 0.13 |
| CL-82574 | 0.32 | 0.62 | 0.63 | 0.52 | 0.18 |
| CL-82458 | 2.18 | 3.43 | 1.66 | 2.42 | 0.91 |
| CL-82658 | 0.22 | 0.31 | 0.21 | 0.25 | 0.05 |
| CL-83083 | 0.47 | 0.43 | 0.34 | 0.41 | 0.07 |
| Isotype Control | — | — | — | — | |
| (IgG4-PE) | | | | | |
| Benchmark 1 | >100 | >100 | >100 | >100 | |
| IgG4-PE | | | | | |

Values are shown as independent experiments (n), average IC₅₀ values and standard deviations (SD) of the average IC₅₀.

### (B) Inhibition of Chemotaxis of Primary Human T-Cells Towards CXCL12

The five anti-CXCR4 antibodies (CL-82558, CL-82458, CL-82658, CL-82574 & CL-83083) (in IgG4-PE format) were tested alongside benchmark anti-CXCR4 antibody Benchmark 1 (in IgG4-PE format), and isotype control antibody human IgG4-PE, in a primary naïve T-cell migration assay using recombinant CXCL12 as chemoattractant. Antibody-induced inhibition of naïve human T-cell migration in response to CXCL12 was evaluated using a trans-well assay and cell counting by flow cytometry.

### (C) Primary Naïve T-Cell Migration Assay

Naïve human T-cells, isolated as described before (Example 9), were resuspended to 10×10⁶ cells/mL in RPMI+1% human AB serum (Sigma) (assay medium) and 50 μL of cell suspension mixed 1:1 with 2× concentrated anti-CXCR4 antibodies (in IgG4-PE format), benchmark antibody Benchmark 1 (in IgG4-PE format) or human isotype control IgG4-PE, in a 96-well U-bottomed plate (Corning Inc.). The antibodies were diluted in assay medium using a 4-fold serial dilution to give a final assay concentration range of 0.029-120 nM and the T-cells pre-treated with the antibodies by incubating for 30 min at 37° C., 5% CO₂. CXCL12 at a final concentration of 6.25 nM was added at 29 μL/well to the bottom chamber on a ChemoTx plate (Neuroprobe Inc.). This was the concentration of CXCL12 chemoattractant estimated to induce peak T-cell migration in a CXCL12 titration curve from 0.781-50 nM using the assay conditions described herein. The framed filter was placed on top of the chemoattractant and 50 μL of the T-cell/antibody mix was added on top of each framed filter, to give a final seeding concentration of 2.5×10⁵ cells/well. The ChemoTx plate was then incubated for 4 h at 37° C., 5% CO2. The migrated T-cells were recovered from the bottom chamber by rinsing once with 25 μL of assay medium. Cell suspensions were then pooled together in a deep-well V-bottomed 96-well plate (Thermo Fisher) and the volume of each well-adjusted to 200 μL with assay medium. Next, the plate was centrifuged at 350×g for 10 min at 4° C. to pellet the cells, which were then fixed by resuspending in 150 μL/well of 4% PFA/PBS for 15 min at room temperature. The plate was then re-centrifuged at 400×g for 10 min at 4° C. and each cell pellet resuspended in 200 μL PBS+2 mM EDTA (Sigma-Aldrich) for analysis of migrated cell counts from a fixed volume of sample using the Attune NxT flow cytometer. Data is expressed as the mean % of max. migration+/−S.D. from triplicate samples. Values are determined by expressing the % migration (Equation 10) as a fraction of the migration response to a concentration of CXCL12 (6.25 nM) estimated to induce peak T-cell migration (Equation 11).

$$Calculation of \% T - cell migration$$

$$\% migration = ( ( Migrated T - cells count - basal migration count ) / ( Input T - cells ) ) \times 100 \qquad Equation 10$$

Migrated cells count=cell counts for T-cells (+/− antibody or human isotype control IgG4-PE pre-treatment) recovered from the lower chamber on the ChemoTx plate using 6.25 nM CXCL12 as chemoattractant

Basal migration count=cell counts for T-cells recovered from lower chamber on the ChemoTx plate using assay medium only as chemoattractant

Input T-cells=Cell counts for the T-cell suspensions added on top of the framed filter

$$Calculation of \% of maximum migration$$

$$Equation 11$$

$$\% of maximum migration = ( ( \% migration ( from Equation 10 ) ) / ( \% migration to 6.25 nM CXCL 12 ) ) \times 100$$

% migration to 6.25 nM CXCL12=number of cells counted for the untreated cells recovered from the lower chamber on the ChemoTx plate using 6.25 nM CXCL12 as chemoattractant.

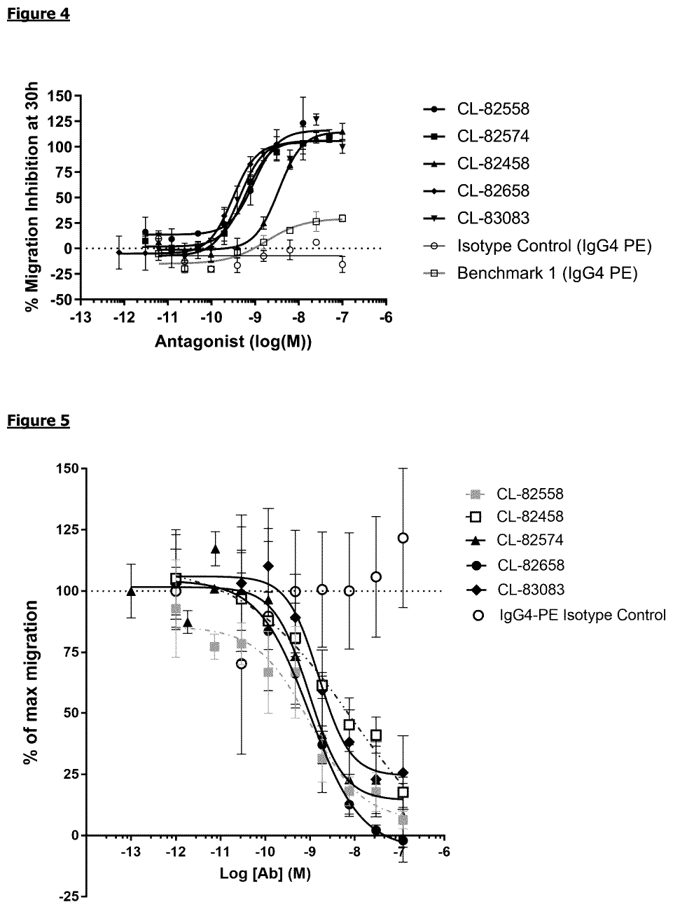

Primary naïve T-cell chemotaxis run at 6.25 nM CXCL12 was inhibited in a concentration-dependent manner by all five anti-CXCR4 antibodies when compared to isotype control human IgG4-PE (FIGS. 5 & 6). Inhibition of chemotaxis in the sub-nanomolar to nanomolar range was achieved for all anti-CXCR4 antibodies (Table 17, FIG. 5). IC₅₀ values could not be accurately determined for CL-82558, CL-82458, Benchmark 1 IgG4-PE due to incomplete curves being generated. Poor maximal inhibition of chemotaxis was shown with Benchmark 1 IgG4-PE, CL-82458 and CL-83083 (FIGS. 5 & 6, Table 17).

TABLE 17
Anti-CXCR4 antibody-mediated inhibition of
naïve T-cell migration to 6.25 nM CXCL12.

| | | % of max | S.D. of | No. |
|---|---|---|---|---|
| Anti-CXCR4 | | migration at | max | of |
| antibody | IC₅₀ (nM) | 120 nM Ab | migration | donors |
| CL-82558 | N.d. - incomplete | 6.34 | 3.65 | 3 |
| | curves | | | |
| CL-82458 | N.d. - incomplete | 17.65 | 6.23 | 4 |
| | curves | | | |
| CL-82574 | 0.95 | 5.44 | 14.64 | 5 |
| CL-82658 | 0.99 | −2.08 | 8.84 | 3 |
| CL-83083 | 1.55 | 25.73 | 15.10 | 4 |
| Benchmark 1 | N.d. - incomplete | 34.17 | 7.99 | 3 |
| IgG4-PE | curves | | | |
| IgG4-PE Isotype | N.d. | 121.70 | 28.47 | 6 |
| Control | | | | |

### (D) Inhibition of CXCL12 Induced Chemotaxis in Isolated Human T-Cells

(i) Pan T-Cell Isolation from Human Blood

Fresh human blood was provided by Cambridge Bioscience. 150-200 mL of blood containing anticoagulant (CPD, citrate phosphate dextrose) was used. Initially the blood was diluted with cold Dulbeccos PBS without calcium or magnesium (Sigma) in 1:2 ratio. 30 mL of diluted blood was carefully layered onto 15 mL Lymphopure (Biolegend) in 50 mL Falcon tubes. Tubes were centrifuged at 800×g for 30 min at room temperature without braking. After the centrifugation, cells from the buffy coat layer, the layer located just above the Lympopure and under the plasma interface, were collected using a Pasteur pipette and placed into a fresh tube. Collected cell suspension was diluted 1:2 with PBS and centrifuged at 340×g for 10 min at room temperature. Cell pellets were resuspended in PBS, pooled into one 50 mL Falcon tube and diluted up to 50 mL with Dulbecco's PBS. Cells were then counted (Nuxcelom Auto T4 on small size cell setting) and centrifuged at 340×g for 10 min. The resulting pellet contained PBMC (peripheral blood mononuclear cells) and was used to isolate T-cells using Pan T-cell Isolation Kit (Miltenyi Biotec). Cell pellet was resuspended in the volume of buffer (PBS without calcium or magnesium+0.5% BSA (Sigma)+2 mM EDTA (Sigma)) calculated as 40 μL per 1×10⁷ cells. Then, 10 μL per 1×10⁷ cells of Pan T-cell Biotin-Antibody Cocktail was added, mixed well and incubated on ice for 5 min. After this time, the cell suspension was further diluted with buffer, 30 μL per 1×10⁷ cells, prior to addition of 20 μL per 1×10⁷ cells of Pan T-cell MicroBead Cocktail. The solution was mixed well and incubated on ice for 10 min. During this incubation, a LS column (Miltenyi Biotec) was inserted into the magnetic field of QurdoMacs Separation Unit (Miltenyi Biotec) and rinsed with 3 mL of buffer. After 10 min incubation, the cell suspension was applied onto the column and the flow-through containing the enriched T-cells was collected. The column was rinse with 2×3 mL of buffer collecting the flow-through into the same tube. This enriched T-cell fraction was resuspended, and the number of cells was counted. A small fraction of cells was taken out to confirm the purity of T-cells by staining the cells with anti CD3 PE conjugated fluorophore antibody (Becton Dickinson) and analysing by flow cytometry using a FACS Canto II (BD BioSciences). Typically, the purity of T-cells was greater than 96%. The remaining cell suspension was centrifuged at 340×g for 5 min and the cells were resuspended in chemotaxis buffer HBSS (Gibco)+0.1% BSA (Sigma).

### (ii) Chemotaxis Using Transwell System

Isolated T-cells were resuspended in chemotaxis buffer to a concentration of ˜40×10⁶ cells/mL in ˜3 mL. Calcein-AM (Invitrogen 11564257) was diluted 1×50 μg vial with 30 μL DMSO and further diluted into 3 mL chemotaxis assay buffer. This 3 mL was added to cell solution and the cells incubated 30 min at 37° C. After this time, cells were then pelleted at 340×g for 5 min and resuspended at 4×10⁶ cells/mL ready for use. Test antibody dilutions including antibodies CL-82558, CL-82458, CL-82658, CL-82574 & CL-83083 (in IgG4-PE format), benchmark antibody Benchmark 1 (in IgG4-PE format) and human IgG4-PE isotype control were prepared in chemotaxis buffer at 10×final assay concentration. Either 5 μL of antibody dilutions in chemotaxis buffer or 5 μL of controls were added to the upper chamber of a chemotaxis plate (HTS transwell 96 well chemotaxis plates 5 μm Corning (via VWR) 734-4081) followed by 45 μL cells. The upper chambers were then placed into spare 96 well plates and incubated for 30 min at 37° C. During this time, 150 μL CXCL12 in chemotaxis buffer at various concentrations was placed into the lower chamber of the chemotaxis plates. After antagonist incubation was complete the upper chamber was carefully lowered into the lower chamber and the plates incubated for 60 min at 37° C. After incubation, the upper chamber was removed and discarded. 100 μL of lower chamber was placed into black 96 well plate and fluorescence measured using the Fluorescence intensity (FI 485 610 520) module on a PHERAstar plate reader.

Assays were performed in a Schild format with single concentrations of antibody tested against a full dose response curve to CXCL12. Data was normalised to calculate % max CXCL12 response using Equation 12, where the largest CXCL12 response was used to define 100% and chemotaxis buffer as 0%.

$$Calculation of percentage max CXCL 12 response$$

$$\% of max CXCL 12 = ( ( sample well - buffer control ) / ( Max CXCL 12 response - buffer control ) ) \times 100 \qquad Equation 12$$

TABLE 18
Anti-CXCR4 antibody-mediated inhibition
of T-cell migration to CXCL12.

| | IC₅₀ of | | % of max | SD of max |
|---|---|---|---|---|
| | antibody | Max | migration | migration |
| | in | Ab | towards 100 nM | towards 100 nM |
| | presence | concen- | CXCL12 in | CXCL12 in |
| | of 30 nM | tration | presence of | presence of |
| | CXCL12 | tested | max antibody | max antibody |
| Antibody | n = 1-5 | (nM) | concentration | concentration |
| CL-82558 | 0.14 | 30 | 16.9 | 9.6 |
| CL-82458 | 0.26 | 300 | 12.9 | 6.5 |
| CL-82574 | 0.21 | 30 | 12.0 | 6.3 |
| CL-82658 | 0.18 | 30 | 0.2 | 3.5 |
| CL-83083 | 0.35 | 100 | 11.7 | 7.7 |
| Benchmark 1 | 1.2 | 100 | 21.9 | 13.6 |
| IgG4-PE | | | | |
| Human | >300 | 300 | 122.6 | 14.2 |
| IgG4-PE | | | | |
| isotype | | | | |
| control | | | | |

### Example 15—Modulation of T-Cell Infiltration into HT-29 Colon Cancer Spheroids Using Anti-CXCR4 Antibodies

3D-spheroid cultures model several in vivo features and structural properties of solid tumours such as cell-to-cell interaction, cell-extracellular matrix interaction and hypoxia conditions. Therefore, they are a useful tool to evaluate T-cell infiltration in vitro. Inhibition of CXCR4 signalling through the neutralisation of CXCL12 ligand is expected to increase the number of T-cells able to infiltrate tumour spheroids. Spheroids composed of the human colon carcinoma cell line (HT-29) were used as a model to assess the ability of anti-CXCR4 antibodies to increase T-cell infiltration into tumour spheroids. The CCRF-HSB-2 T-cell line, which express both CXCR4 and CXCR3 receptors, was used as a model of primary T-cells. HT-29 cells were shown to secrete CXCR3 ligands (CXCL9, CXCL10 and CXCL11) when stimulated with interferon gamma (IFNγ). Spheroids were formed over 3 days in culture and recombinant CXCL12 ligand was then added to the assay media to form a high concentration of CXCL12 surrounding the individual spheroids. Consequently, T-cell chemotaxis towards CXCR3 ligands was disrupted and resulted in T-cells to undergo chemokinesis, where directional movement towards a gradient is lost. Inhibition of the CXCL12 signalling by anti-CXCR4 antibodies resulted in the restoration of the CXCR3 chemotaxis of T-cells towards CXCL9, CXCL10 and/or CXCL11 released by the spheroids and resulted in increased levels of infiltration. This model of infiltration can also be set up using primary T-cells isolated from human blood (e.g. fraction of PBMC or further isolated to pan CD3⁺ or CD4⁺/CD8⁺), that has the potential to assess both T-cell infiltration and T-cell mediated killing of tumour cells in response to anti-CXCR4 antibodies either as monotherapies and/or in combination with checkpoint inhibitors such as anti-PD1/PD-L1 or anti-CTLA-4.

HT-29 (ATCC HTB-38) tumour cells were resuspended to 5.0×10⁴ cells/mL in McCoy5a (Gibco)+10% FBS (assay media) and 100 μL/well of cell suspension were added to a U-bottom ultra-low attachment assay plate (Corning). Recombinant human IFNγ (R&D) was diluted to 15 ng/mL in assay media and 50 μL/well were added to assay plate to give a final concentration of 5 ng/mL. Assay plate was centrifuged at 120×g for 10 min and incubated at 37° C., 5% CO₂ for 3 days to allow a single spheroid per well to form. On day 3, antibodies were diluted at 16× in assay media using a 4-fold serial dilution to give a concentration range of 1600 nM-97.6 μM (final assay concentration range of 100 nM-6.1 μM) and 100 μL of antibody titration were mixed with 100 μL of CXCL12 diluted to 80 nM in assay media (final assay concentration 5 nM). This concentration of CXCL12 was estimated to induce the maximum inhibition of CCRF-HSB-2 infiltration as per CXCL12 titration curve prepared from 20 nM-1.2 μM using the assay conditions described herein. CCRF-HSB-2 (ATCC CCL120.1) cells were washed twice with PBS, resuspended to 1.0×10⁶ cells/mL in PBS supplemented with 1 μM CellTrace™ Far Red labelling dye (Thermo Fisher) and incubated for 20 min at 37° C., 5% CO₂. Assay media was then added to the cells (minimum 0.6 times initial labelling volume) and cell suspension was incubated for 5 min at 37° C., 5% CO₂ to remove any free dye remaining. Cells were centrifuged at 300×g for 5 min and resuspended to 2.0×10⁶ cells/mL in assay media. 25 μL/well of antibody/CXCL12 mix and 25 μL/well of labelled CCRF-HSB-2 cells were added to assay plate, which was then incubated overnight at 37° C., 5% CO2. Each assay plate included positive control wells (no CXCL12, no antibody) and negative control wells (5 nM CXCL12, no antibody) of CCRF-HSB-2 infiltration. On day 4, assay plate was removed from the incubator and spheroids were washed with 150 μL/well of assay media by pipetting up and down 3 times. 150 μL/well of PBS were added to spheroids, which were washed a second time, as described previously, to remove CCRF-HSB-2 cells that failed to infiltrate the HT-29 tumour spheroids. 150 μL/well of PBS were added to washed spheroids and assay plate was immediately scanned in the IncuCyte™ S3 Live Cell Analysis System using the spheroid scan mode and a 10×objective capturing images both in the brightfield and the red fluorescent channels.

Images acquired by the IncuCyte™ S3 Live Cell Analysis System were analysed using a Spheroid analysis module on the IncuCyte™ S3 software. Using the brightfield images, parameters were selected to define spheroid objects in an accurate way (segmentation sensitivity of 60 and minimal area filter of 1×10⁴ μm²). Images acquired in the red fluorescent channel were analysed using a background subtraction algorithm that estimate the fluorescence background across the images using a disk of 700 μm radius. The estimated background was then subtracted from the original fluorescence values resulting in background corrected images. Using the Graph Metrics on the IncuCyte™ S3 software, the mean red fluorescence intensity within the largest brightfield object boundary (i.e. spheroid) was calculated for each well and values were exported to be further analysed using Excel and GraphPad Prism software. For each assay plate, averages of positive control wells (maximum infiltration) and negative control wells (minimum infiltration) were calculated and % of infiltration in response to anti-CXCR4 antibody treatment was calculated as described in Equation 13. For each antibody tested, the average percentage of infiltration from 5 independent experiments was plotted in GraphPad Prism against Log of concentration expressed in molarity (FIG. 7). Percentage of maximum infiltration observed at the top antibody concentration tested (100 nM) was summarised in Table 19.

$$Calculation of percentage ( \% ) of infiltration$$

$$\% infiltration = ( ( signal - mean of negative controls ) / ( mean of positive controls - mean of negative controls ) ) \times 100 \qquad Equation 13$$

Infiltration of CCRF-HSB-2 cells into IFNγ-treated HT-29 tumour spheroids was inhibited by the presence of 5 nM CXCL12, and treatment with CL-82574 and CL-83083-2 anti-CXCR4 antibodies (in IgG4-PE format) resulted in concentration-dependent increase of CCRF-HSB-2 infiltration when compared to the human IgG4-PE isotype control. Increase of infiltration by both antibodies was observed to be in the nanomolar range, with the percentage of maximum infiltration at the top antibody concentration tested (100 nM) reaching 88.58% for CL-82574 and 71.54% for CL-83083-2 (Table 19, FIG. 7).

TABLE 19
Anti-CXCR4 antibody-mediated percentage of maximum infiltration
of CCRF-HSB-2 cells into IFNγ-treated HT-29 tumour
spheroids, in the presence of 5 nM CXCL12.

| | | | | | | Mean % | SD % |
|---|---|---|---|---|---|---|---|
| | | | | | | of max | of max |
| | % of max infiltration at 100 nM | | | | | infil- | infil- |
| Antibodies | n = 1 | n = 2 | n = 3 | n = 4 | n = 5 | tration | tration |
| CL-82574 | 75.43 | 88.06 | 100.78 | 86.31 | 92.32 | 88.58 | 9.24 |
| CL-83083-2 | 61.03 | 77.02 | 85.38 | 51.9 | 82.35 | 71.54 | 14.44 |
| Isotype | 15.56 | −3.09 | 9.03 | 4.67 | −2.78 | 4.68 | 7.96 |
| Control | | | | | | | |
| (IgG4-PE) | | | | | | | |

Values are shown as independent experiments (n), average % of maximum infiltration and standard deviations (SD) of percentage of maximum infiltration.

### Example 16—Mobilisation of CD45⁺ Cells from Bone Marrow of Humanised CD34⁺ NSG Mice

Twenty female hNSG-CD34 animals were imported from Jackson Laboratories in the US. Upon arrival they were housed in groups of 5 animals per cage within transparent plastic IVC cages with a wire hopper (Green line GM500 Sealsafe Plus, Tecniplast UK). On day-1 animals were weighed to get a pre-study weight with the study only commencing when the animals were over 18 g in body weight. Animals were implanted with an identification microchip.

On the day of the study animals were injected intraperitoneal (I.P.) with 100 μL PBS or 100 μL of anti-CXCR4 antibodies CL-82574 or CL-83083-2 (in IgG4-PE format) (2 mg/mL in saline), equivalent to 10 mg/kg for a 20 g mouse. Blood was taken by a terminal bleed under anaesthetic with isoflurane, 1 h after treatment, and the blood was stored in EDTA-coated tubes on ice until analysis. Animals were dosed with either 100 μL of PBS or antibody as shown in Table 20.

TABLE 20
Group assignments for the study indicating the number
of mice used per group and treatment received.

| | Number of | |
|---|---|---|
| Group | animals | Treatment |
| 1 | 5 | PBS |
| 2 | 5 | CL-82574 (10 mg/kg) |
| 3 | 5 | CL-83083-2 (10 mg/kg ) |

### (A) Blood Processing for Flow Cytometry

To prepare for staining, 500 μL of blood was added to 4.5 mL of red blood cell lysis buffer (Sigma, #R7757) in a 15 mL falcon tube and incubated at room temperature for 10 min. After the incubation, the tubes were centrifuged at 300×g for 5 min at room temperature. The pellet was resuspended in 5 mL FACS buffer (PBS+1% BSA (Sigma #A7906)+0.1% sodium azide (Severn Biotec #40-2010-10)+2 mM EDTA (Sigma #03690)) and centrifuged again at 300×g for 5 min at room temperature. The cell pellet was then resuspended in 200 μL of FACS buffer and kept on ice.

For the flow cytometry analysis, the entire 200 μL of each blood sample were transferred to a 96 well polypropylene deep well plate (Corning #3957). The plate was centrifuged at 300×g for 5 min at 4° C., then cells were re-suspended in 50 μL of Fc block master mix (1 μL mouse Trustain+2.5 μL human Trustain: 50 μL FACS buffer, Biolegend #101320 and #422302) and incubated at 4° C. for 10 min. Finally, the cells were incubated for 30 min with 50 μL of flow cytometry antibodies as shown in the master mix panels (Table 21). After staining, cells were washed twice in FACS buffer to remove any unbound antibodies. Cells were fixed using 4% paraformaldehyde for 20 min at room temperature, and then washed in PBS before all the samples were resuspended in 200 μL of PBS until data acquisition. Data was acquired using an Attune NxT Flow Cytometer.

TABLE 21
Flow cytometry panel of markers used
for CD45 staining of mobilised cells.

| Marker | Supplier | Cat. # | Fluorophore |
|---|---|---|---|
| CD45 | E-bioscience | 48-0459-42 | eFluor 450 |
| CD34 | Biolegend | 343506 | PE |
| Mouse Fc/Block | Biolegend | 101320 | — |
| Human Fc/Block | Biolegend | 422302 | — |

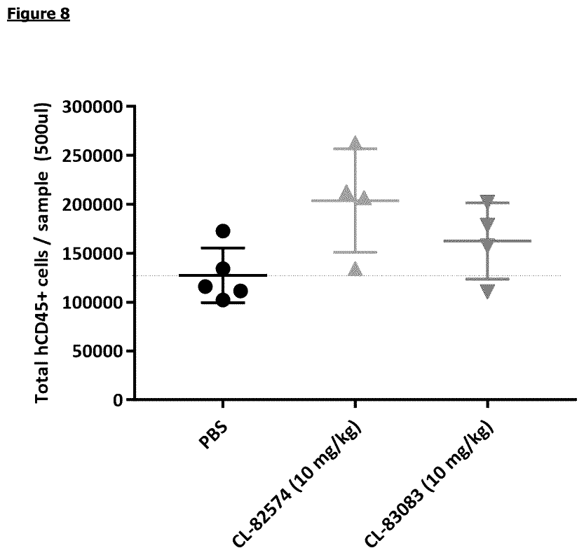

FIG. 8 shows total numbers of human CD45+ cells detected in the well of each blood sample, as recorded by the flow cytometer. There is a notable increase in the mean leukocyte counts for all treated groups compared to the control, especially the CL-82574 group.

### Example 17—Mobilisation of Leukocytes from Bone Marrow in Cynomolgus Monkey

To determine the effect on mobilisation of white blood cells from the bone marrow it is warranted to carry out a mobilisation study using cynomolgus monkeys with CXCR4 antibody. Mobilisation studies will be carried out by a similar method as described by Peng et al., Distinct mobilization of leukocytes and hematopoietic stem cells by CXCR4 peptide antagonist LY2510924 and monoclonal antibody LY2624587 (2017) Oncotarget. 8(55):94619-94634. Briefly, Male and female cynomolgus monkeys aged 2 to 4 years and weight 2.5 to 4 kg will be subcutaneously dosed with selected CXCR4 antibodies (10-30 mg/kg single injection). Whole blood samples will be collected from each animal pre-dose for baseline counts, and post-dose at multiple time points up to 24 h post dose (day 1) and day 4 for cell counts. WBCs and neutrophils will be analysed via routine blood analysis in a clinical pathology laboratory. Counts of CD34⁺ cells in peripheral blood will be calculated using flow cytometry using a method similar to published protocols (e.g. Peng et al., 2017). For example, to calculate numbers of CD34⁺ cells within a sample of whole blood; 20 μL of a stock staining solution (⅓ anti-CD45-perCP, ⅓ anti-CD34-PE, and ⅓ live/dead stain) will be added to tubes with calibration beads for cell quantitation. Tubes containing stock staining solution will be diluted with 25 μL whole blood plus 25 μL RPMI 1640+0.1% BSA, incubated for 15 min at room temperature in the dark, followed by addition of lysis solution to lyse red blood cells, incubation for 30 min, then flow cytometry analysis with a suitable flow cytometer. Gating/region strategies will be defined by side scatter (SS) versus nuclear dye (lymphocytes) and SS versus CD45 (CD45-negative/dim cells), and combined regions will be viewed in a dot plot of CD34 versus nuclear dye (counting both CD34-negative/dim and CD34⁺ cells in the final plot). Beads will be defined in the SS versus per CP plot to count CD34⁺ cells/μL whole blood (Equation 14). Inhibition of CXCR4 by antibody treatment is expected to result in 2-6-fold increase in described cell types over time course. (number of CD34+ cells)/(number of beads)×(beads per test)/(test volume)×dilution factor

$$Calculation of CD 34 + cells per μL blood$$

$$CD {34}^{+} cells / μL = ( number of CD 34 + cells ) / ( number of beads ) \times ( beads per test ) / ( test volume ) \times dilution factor \qquad Equation 14$$

### Example 18—Tumour Efficacy and CD45+ Cell Mobilisation Studies Using Human CXCR4 Knock-In Mice

Efficacy studies will be performed using MC38, LLC or B16-F10 tumour models implanted in human CXCR4 knock-in C57BL/6 mice. On the day of implantation, tumours will be injected subcutaneously into mice on the rear right flank with 1×10⁵ tumour cells/animal. Treated groups will receive their first dose of antibody (Table 22) or control antibodies (antibodies dosed intraperitoneally at 10 mg/kg. Six days post-implantation of the tumour cells, the mice will be dosed with antibodies three times a week, for a total of two weeks. Tumour development will be monitored three times a week using digital calipers measuring in two dimensions until end of the study. Tumour volumes (mm³) will be estimated using Equation 15.

$$Calculation of tumour volume$$

$$Tumour volume ( mm 3 ) = \frac{L \times W 2}{2} \qquad Equation 15$$

With L being the larger diameter, and W the smaller diameter of the tumour.
Mice are kept on-study until their tumours develop to a mean diameter of 12 mm or they reach the humane endpoint outlined in the study protocol. The humane endpoint survival statistics will be calculated using the Kaplan-Meier method with Prism (GraphPad).

TABLE 22
Treatment groups of human CXCR4 knock-in mice.

| | Number of | |
|---|---|---|
| Group | animals | Treatment |
| 1 | 5 | 10 mg/kg isotype control human IgG4-PE |
| 2 | 5 | 10 mg/kg CL-82574 |
| 3 | 5 | 10 mg/kg Anti-PD-L1 antibody |
| 4 | 5 | 10 mg/kg CL-82574 + 10 mg/kg Anti-PD-L1 |
| | | antibody |

### (B) CD45+ Cell Mobilisation Studies Using Human CXCR4 Knock-In Mice

The protocol will be carried out as described in Example 16. Briefly, on the day of the study, human CXCR4 knock-in mice will be injected Intraperitoneal with 100 μL PBS or 100 μL of anti-CXCR4 antibody (2 mg/mL in saline), equivalent to 10 mg/kg for a 20 g mouse. Blood will be taken by a terminal bleed under anaesthetic with isoflurane, 1 h after treatment, and the blood stored in EDTA-coated tubes on ice until analysis. Analysis will be carried out using a similar method as described in Example 16.

TABLE 23
Antibody gene segment table

| | | | | | Non- | Mutations | | | | | Non- | Mutations | |
|---|---|---|---|---|---|---|---|---|---|---|---|---|---|
| | | | | | germline | outside CDRH3 | | | | | germline | outside CDRL3 | |
| | IGHV | IGHD | IGHJ | CDRH3 | CDRH3 | Nucleic | Amino | IGLV | IGLJ | CDRL3 | CDRH3 | Nucleic | Amino |
| AntibodyID | gene | gene | gene | length | Amino acid | acid | acid | gene | gene | length | Amino acid | acid | acid |
| CL- | IGHV3- | IGHD3- | IGHJ6 | 19 | 8 | 2 | 2 | IGKV1- | IGKJ3 | 9 | 1 | 1 | 1 |
| 82658 | 30*18 | 16*02 | *02 | | | | | 17*01 | *01 | | | | |
| CL- | IGHV3- | IGHD3- | IGHJ6 | 19 | 6 | 9 | 3 | IGLV10- | IGLJ3 | 11 | 1 | 4 | 3 |
| 83083 | 73*02 | 9*01 | *02 | | | | | 54*02 | *02 | | | | |
| CL- | IGHV4- | IGHD3- | IGHJ6 | 22 | 11 | 7 | 5 | IGLV3- | IGLJ3 | 11 | 1 | 2 | 2 |
| 83158 | 34*01 | 10*01 | *02 | | | | | 19*01 | *02 | | | | |
| CL- | IGHV3- | IGHD3- | IGHJ6 | 19 | 6 | 9 | 3 | IGLV10- | IGLJ3 | 11 | — | — | — |
| 83083-2 | 73*02 | 9*01 | *02 | | | | | 54*02 | *02 | | | | |
| CL- | IGHV3- | IGHD1- | IGHJ6 | 17 | 4 | 10 | 9 | IGKV1- | IGKJ4 | 9 | 0 | 0 | 0 |
| 82523 | 23*04 | 20*01 | *02 | | | | | 17*01 | *01 | | | | |
| CL- | IGHV4- | IGHD3- | IGHJ6 | 21 | 8 | 14 | 10 | IGKV2- | IGKJ1 | 9 | 0 | 6 | 4 |
| 82558 | 31*03 | 10*01 | *02 | | | | | 28*01 | *01 | | | | |
| CL- | IGHV3- | IGHD1- | IGHJ3 | 14 | 8 | 8 | 5 | IGKV1- | IGKJ3 | 9 | 0 | 3 | 2 |
| 82574 | 33*01 | 20*01 | *02 | | | | | 17*01 | *01 | | | | |
| CL- | IGHV3- | IGHD1- | IGHJ3 | 14 | 8 | 8 | 4 | IGKV1- | IGKJ3 | 9 | 0 | 5 | 4 |
| 82556 | 33*01 | 20*01 | *02 | | | | | 17*01 | *01 | | | | |
| CL- | IGHV3- | IGHD1- | IGHJ3 | 14 | 7 | 6 | 4 | IGKV1- | IGKJ3 | 9 | 0 | 3 | 2 |
| 82583 | 33*01 | 1*01 | *02 | | | | | 17*01 | *01 | | | | |
| CL- | IGHV3- | IGHD1- | IGHJ6 | 17 | 3 | 18 | 10 | IGKV1- | IGKJ4 | 9 | 1 | 7 | 3 |
| 82458 | 23*04 | 1*01 | *02 | | | | | 17*01 | *01 | | | | |
| CL- | IGHV3- | IGHD1- | IGHJ6 | 17 | 6 | 5 | 4 | IGKV1- | IGKJ1 | 9 | 1 | 3 | 1 |
| 82562 | 30*18 | 20*01 | *02 | | | | | 17*01 | *01 | | | | |
| CL- | IGHV4- | IGHD3- | IGHJ6 | 21 | 8 | 18 | 14 | IGKV2- | IGKJ1 | 9 | 0 | 3 | 3 |
| 82577 | 31*03 | 10*01 | *02 | | | | | 28*01 | *01 | | | | |
| CL- | IGHV3- | IGHD5- | IGHJ6 | 17 | 5 | 2 | 2 | IGKV1D- | IGKJ4 | 9 | 0 | 1 | 0 |
| 82517 | 21*03 | 18*01 | *02 | | | | | 13*d01 | *01 | | | | |
| CL- | IGHV4- | IGHD3- | IGHJ6 | 21 | 7 | 11 | 8 | IGKV2- | IGKJ1 | 9 | 0 | 2 | 2 |
| 82541 | 31*03 | 10*01 | *02 | | | | | 28*01 | *01 | | | | |
| CL- | IGHV3- | IGHD3- | IGHJ4 | 15 | 5 | 6 | 3 | IGKV2- | IGKJ3 | 9 | 0 | 0 | 0 |
| 82473 | 33*01 | 10*01 | *02 | | | | | 30*01 | *01 | | | | |
| CL- | IGHV1- | IGHD3- | IGHJ6 | 23 | 7 | 0 | 0 | IGKV1- | IGKJ1 | 9 | 0 | 3 | 3 |
| 82551 | 8*01 | 10*01 | *02 | | | | | 5*03 | *01 | | | | |
| CL- | IGHV1- | IGHD3- | IGHJ6 | 23 | 6 | 4 | 3 | IGKV1- | IGKJ1 | 9 | 0 | 1 | 1 |
| 82580 | 8*01 | 10*01 | *02 | | | | | 5*03 | *01 | | | | |
| CL- | IGHV3- | IGHD3- | IGHJ6 | 19 | 6 | 9 | 3 | IGKV1- | IGKJ1 | 9 | 1 | 3 | 2 |
| 82571 | 73*02 | 9*01 | *02 | | | | | 5*03 | *01 | | | | |
| CL- | IGHV3- | IGHD3- | IGHJ6 | 23 | 6 | 8 | 5 | IGKV1- | IGKJ1 | 9 | 0 | 0 | 0 |
| 82495 | 33*01 | 10*01 | *02 | | | | | 17*01 | *01 | | | | |
| CL- | IGHV3- | IGHD3- | IGHJ6 | 23 | 9 | 6 | 5 | IGKV3- | IGKJ1 | 9 | 0 | 4 | 4 |
| 82455 | 33*01 | 10*01 | *02 | | | | | 15*01 | *01 | | | | |
| CL- | IGHV3- | IGHD1- | IGHJ6 | 17 | 3 | 14 | 8 | IGKV1- | IGKJ4 | 9 | 1 | 6 | 3 |
| 148712 | 23*04 | 1*01 | *02 | | | | | 17*01 | *01 | | | | |
| CL- | IGHV3- | IGHD6- | IGHJ4 | 6 | 0 | 11 | 8 | IGLV1- | IGLJ3 | 12 | 4 | 8 | 5 |
| 148729 | 73*02 | 25*01 | *02 | | | | | 51*01 | *02 | | | | |

### Example 19: In Vivo Anti-Tumour Efficacy of an Anti-CXCR4 Antibody

To investigate the in vivo anti-tumour efficacy of CL-82574, a study was performed using the syngeneic tumour model Pan02 (Crown Bioscience Ltd, UK). For this in vivo work, C57BL/6 mice were genetically modified to replace the mouse CXCR4 gene with its human counterpart, and CL-82574 was reformatted as mouse IgG1. The antibody was administered either as a monotherapy or in combination with anti-PD-1 (BioXcell, RMP1-14),

Mice were maintained at the Biological Support Unit of Babraham Institute (Cambridge, UK) and supplied to Crown Bioscience Ltd at 8-11 weeks of age. Before the beginning of the study, mice were randomly allocated to treatment groups (12 mice per group, 3 males and 9 females), see Table 24. Pan02 cells were passaged in vitro using RPMI supplemented with 10% foetal calf serum and 2 mM L-glutamine. Cell viability was confirmed to be above 90% at the time of tumour cell injection. Pan02 cells were resuspended in PBS and subcutaneously injected into the left flanks of mice (3×10⁶ cells/mouse). Mice were dosed intraperitoneally (i.p.) with 45 mg/kg mIgG1 CL-82574 (Q3D starting from day 8) for three weeks either as monotherapy or in combination with 10 mg/kg anti-PD-1 (Q3D, starting from day 15). Mice in the control group were treated with phosphate buffered saline (PBS), as a vehicle control.

TABLE 24
Treatment groups for the in vivo anti-tumour
efficacy study in the Pan02 model

| | | | | Dose | | Start- | |
|---|---|---|---|---|---|---|---|
| | N | Treat- | Dose | Volume | Regi- | ing | |
| Group | Mice | ment | (mg/kg) | (ml/kg) | men | day | Route |
| Vehicle | 12 | PBS | 0 | 10 | Q3D for | 8 | i.p. |
| | | | | | 3 weeks | | |
| Vehicle + | 12 | PBS | 0 | 10 | Q3D for | 8 | i.p |
| anti-PD1 | | | | | 3 weeks | | |
| | | ratIgG2a- | 10 | 10 | Q3D for | 15 | i.p. |
| | | anti-PD-1 | | | 2 weeks | | |
| CL- | 12 | CL-82574 | 45 | 10 | Q3D for | 8 | i.p. |
| 82574 + | | | | | 3 weeks | | |
| anti-PD1 | | ratIgG2a | 10 | 10 | Q3D for | 15 | i.p. |
| | | anti-PD-1 | | | 2 weeks | | |
| CL- | 12 | CL-82574 | 45 | 10 | Q3D for | 8 | i.p |
| 82574 | | | | | 3 weeks | | |

Tumour size and mouse bodyweight were measured 3 times a week from the day of tumour cell injection. Mice were kept on study until their tumour reached an average diameter of 15 mm, or when displayed tumour ulceration (welfare endpoint). The study was terminated on day 60. Tumour volume was calculated using the modified ellipsoid formula: 0.5×(Length×Width²). Statistical analysis was performed on tumour volumes from day 25 to day 44, later dates were not considered for the analysis due to the high proportion of missing values (endpoint reached).

Monotherapies, with either CL-82574 or anti-PD-1, did not show anti-tumour effect in this model. In contrast, mice receiving a combinatorial treatment of CL-82574 and anti-PD-1 displayed a delayed tumour growth when compared to the vehicle control as well as the anti-PD-1 monotherapy. The difference observed was analysed with a Two-Way ANOVA-Type, performed on the tumour volume changes from baseline (set at day 7, before the start of the dosing). Tumour growth data obtained are summarised in FIG. 11. Adjusted p-values are shown in Table 25.

These data suggest a combinatorial anti-tumour effect between CL-82574 and anti-PD-1 therapy.

TABLE 25
Comparison of each treatment group versus the vehicle group and comparison of CL-82574 +
aPD1 versus Vehicle + anti.PD1 between day 25 and day 44 post tumour implant
Adjusted P-values

| Group 1 | Group 2 | Day 25 | Day 28 | Day 30 | Day 32 | Day 35 |
|---|---|---|---|---|---|---|
| Vehicle | Vehicle + anti-PD-1 | 1.0000 | 1.0000 | 0.4061 | 0.9052 | 0.1949 |
| Vehicle | CL-82574 + anti-PD-1 | 0.0836 | 0.0364 | 0.0002 | 0.0163 | 0.0005 |
| Vehicle | CL-82574 | 1.0000 | 1.0000 | 0.4735 | 0.9052 | 0.7793 |
| Vehicle + anti-PD-1 | CL-82574 + anti-PD-1 | 0.2921 | 0.0725 | 0.0170 | 0.0018 | 0.0748 |

| Group 1 | Group 2 | Day 37 | Day 39 | Day 42 | Day 44 |
|---|---|---|---|---|---|
| Vehicle | Vehicle + anti-PD-1 | 0.0860 | 0.5666 | 0.8308 | 1.0000 |
| Vehicle | CL-82574 + anti-PD-1 | <.0001 | 0.0011 | 0.0099 | 0.0180 |
| Vehicle | CL-82574 | 0.9090 | 0.5666 | 0.8308 | 1.0000 |
| Vehicle + anti-PD-1 | CL-82574 + anti-PD-1 | 0.0398 | 0.0243 | 0.0013 | 0.0193 |

Adjusted p-values obtained using Bonferroni-Holm correction by day after a global two-way ANOVA-type including all groups with factors Treatment and day (repeated) and their interactions followed by contrasts analysis at each day.

1. An antibody or antigen-binding fragment thereof which specifically binds to CXCR4, wherein the antibody or antigen-binding fragment thereof comprises a VH domain, wherein the VH domain comprises:
i. a CDRH1 amino acid sequence of SEQ ID NOs: 10 or 13;
ii. a CDRH2 amino acid sequence of SEQ ID NOs: 11 or 14;
iii. a CDRH3 amino acid sequence of SEQ ID NOs: 12 or 15; and
wherein the antibody or antigen-binding fragment thereof comprises a VL domain,
wherein the VL domain comprises:
i. a CDRL1 amino acid sequence of SEQ ID NOs: 20 or 23;
ii. a CDRL2 amino acid sequence of SEQ ID NOs: 21 or 24; and
iii. a CDRL3 amino acid sequence of SEQ ID NOs: 22 or 25.

2. The antibody or fragment according to claim 1, wherein
the V_{H} domain comprises an amino acid sequence of SEQ ID NO: 16; and
the V_{L} domain comprises an amino acid sequence of SEQ ID NO: 26.

3. The antibody or fragment according to claim 1, wherein:
a) the CXCR4 is human (optionally selected from SEQ ID NO: 1, SEQ ID NO: 2 and SEQ ID NO: 3);
b) the CXCR4 is rhesus and/or cynomolgus (optionally selected from SEQ ID NO: 7, SEQ ID NO: 8 and SEQ ID NO: 9); or
c) the antibody or fragment specifically binds to human, rhesus, cynomolgus and/or rodent CXCR4, optionally human and cynomolgus CXCR4;
optionally wherein the binding is determined by surface plasmon resonance (SPR), flow cytometry, live cell imaging, ELISA or radioligand binding.

4. The antibody or fragment according to claim 1, wherein the antibody or fragment comprises a constant region (CH and/or CL), optionally wherein:
a) the C_{H} is (i) an IgG4 constant region, such as an IgG4-PE constant region (e.g. SEQ ID NO: 267 or SEQ ID NO: 305) or (ii) an IgG1 constant region, such as an IgG1 constant region comprising mutations that reduce binding to Fc-7 receptors and/or C1q as compared to wild-type (e.g. SEQ ID NO: 249 or 307); and/or
b) the C_{L} is a kappa light chain constant region.

5. The antibody or fragment as defined in claim 1, wherein the antibody comprises a heavy chain and a light chain, and the heavy chain comprises an amino acid sequence of SEQ ID NO: 18 and the light chain comprises an amino acid sequence of SEQ ID NO: 28.

6. The antibody or fragment as defined in claim 1, wherein the antibody or fragment inhibits the binding of CXCL12 to CXCR4.

7. The antibody or fragment as defined in claim 1, wherein the antibody or fragment does not induce apoptosis in T-cells (optionally CD8+ T-cells), optionally wherein apoptosis is determined using flow cytometry.

8. The antibody or fragment as defined in claim 1, wherein:
a) the antibody or fragment binds to cynomolgus CXCR4 with an EC50 of from 0.5 to 10 nM (e.g. 0.5 to 5 nM), optionally wherein cynomolgus CXCR4 binding is determined using flow cytometry;
b) the antibody or fragment binds to human CXCR4 with a K_{D} of from 0.2 to 2 nM (e.g. 0.4 to 0.8 nM), optionally wherein binding affinity is determined using surface plasmon resonance (SPR);
c) the antibody or fragment does not bind to CXCR7 (optionally wherein CXCR7 is human and is further optionally selected from SEQ ID NO: 4, SEQ ID NO: 5 or SEQ ID NO: 6), optionally wherein CXCR7 binding is determined using flow cytometry, radioligand binding, surface plasmon resonance (SPR), live cell imaging or ELISA;
d) the antibody or fragment inhibits CXCL12 mediated response of T-cells with an IC50 of from 0.5 to 20 nM (e.g. 0.5 to 5 nM), wherein the inhibition is determined using label free dynamic mass redistribution assay in vitro; and/or
e) the antibody or fragment inhibits chemotaxis of CXCR4⁺ T-cells to CXCL12 with an IC₅₀ of from 0.01 to 5 nM (e.g. 0.01 to 1 nM), wherein the inhibition is determined using live cell imaging or flow cytometry.

9-12. (canceled)

13. The antibody or fragment as defined in claim 1, wherein the antibody or fragment increases mean CD45⁺ cell mobilisation compared to PBS, optionally wherein mobilisation is determined using flow cytometry.

14. The antibody or fragment as defined in claim 1, wherein the antibody or fragment binds to CXCR4 homodimers.

15. The antibody or fragment as defined in claim 1, wherein the antibody or fragment enables CD8⁺ T-cells to infiltrate a tumour.

16-20. (canceled)

21. A pharmaceutical composition comprising an antibody or fragment as defined in claim 1 and a pharmaceutically acceptable excipient, diluent or carrier and optionally further comprising one or more further therapeutic agents.

22. A nucleic acid that encodes (a) a V_{H} domain and/or a V_{L} domain of an antibody or fragment as defined in claim 1 or (b) a heavy chain and/or a light chain of an antibody or fragment as defined in claim 1.

23. A vector comprising the nucleic acid as defined in claim 22; optionally wherein the vector is a CHO or HEK293 vector.

24. A host cell comprising the nucleic acid of claim 22 or the vector as defined in claim 23.

25. A method of treating or preventing a CXCR4-mediated disease or condition in a patient, the method comprising administering to said patient a therapeutically or prophylactically effective amount of the antibody or fragment as defined in claim 1, wherein the CXCR4-mediated disease or condition is thereby treated or prevented.

26. The method of claim 25, wherein the CXCR4-mediated disease or condition is cancer, optionally wherein the cancer is pancreatic cancer or pancreatic ductal cancer.

27. The method of claim 25, wherein the CXCR4-mediated disease or condition is a solid tumor.

28. The method of claim 25, wherein the treatment further comprises administering a further therapy, optionally wherein the further therapy comprises one or more further therapeutic agent(s) independently selected from the group consisting of an anti-PD-1 antibody or antigen-binding fragment thereof and an anti-PD-L1 antibody or antigen-binding fragment thereof, and/or optionally wherein the further therapy is selected from chemotherapy, radiotherapy and/or surgical removal of tumors.

29. The method of claim 25, further comprising administering a further therapeutic agent which is a PD-1/PD-L1 signalling inhibitor (e.g. a PD-1 antibody or antigen-binding fragment thereof, or a PD-L1 antibody or antigen-binding fragment thereof).
